(19) United States
(12) Patent Application Publication
(10) Pub. No.: US 2024/0287080 A1
(43) Pub. Date: Aug. 29, 2024

(54) O-LINKED THIADIAZOLYL COMPOUNDS AS DNA POLYMERASE THETA INHIBITORS

(71) Applicants: IDEAYA Biosciences Inc., South San Francisco, CA (US); GlaxoSmithKline Intellectual Property (No.4) Limited, Stevenage (GB)

(72) Inventors: Paul A. BARSANTI, South San Francisco, CA (US); Kevin J. DUFFY, Collegeville, PA (US); Brian G. LAWHORN, Downingtown, PA (US); Firoz Ali JAIPURI, Fremont, CA (US); Daneil Lee SEVERANCE, San Diego, CA (US); Chenbo WANG, South San Francisco, CA (US); Nicholas David ADAMS, Royersford, PA (US); Janos BOTYANSZKI, Collegeville, PA (US); Michael G. DARCY, Collegeville, PA (US); Terence J. KIESOW, Collegeville, PA (US); John J. MCATEE, Collegeville, PA (US); Cuthbert MARTYR, Collegeville, PA (US); Alexander BUTTRAGO SANTANILLA, Collegeville, PA (US); Xinrong TIAN, Collegeville, PA (US)

(21) Appl. No.: 18/567,687

(22) PCT Filed: Jun. 9, 2022
(86) PCT No.: PCT/IB22/55384

§ 371 Date: Dec. 6, 2023

Related U.S. Application Data
(60) Provisional application No. 63/209,555, filed on Jun. 11, 2021.
(60) Provisional application No. 63/319,826, filed on Mar. 15, 2022.

(51) Int. Cl. C07D 487/04; A61K 31/437; A61K 31/4439; A61K 31/444; A61K 31/519; C07D 417/12; C07D 417/14; C07D 471/04; C07D 493/04; C07D 498/04

(52) U.S. Cl. CPC C07D 487/04; A61K 31/437; A61K 31/4439; A61K 31/444; A61K 31/519; C07D 417/12; C07D 417/14; C07D 471/04; C07D 493/04; C07D 498/04

(57) ABSTRACT

Disclosed herein are certain thiadiazolyl derivatives Formula (I):

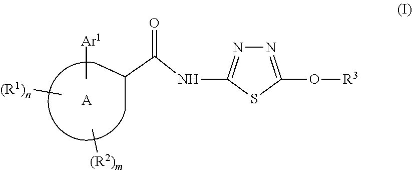

that inhibit DNA Polymerase Theta (Polθ) activity, in particular inhibit Polθ activity by inhibiting ATP dependent helicase domain activity of Polθ. Also, disclosed are pharmaceutical compositions comprising such compounds and methods of treating and/or preventing diseases treatable by inhibition of Polθ such as cancer, including homologous recombination (HR) deficient cancers.

### BACKGROUND

Targeting DNA repair deficiencies has become a proven and effective strategy in cancer treatment. However, DNA repair deficient cancers often become dependent on backup DNA repair pathways, which present an “Achilles heel” that can be targeted to eliminate cancer cells, and is the basis of synthetic lethality. Synthetic lethality is exemplified by the success of poly (ADP-ribose) polymerase (PARP) inhibitors in treating BRCA-deficient breast and ovarian cancers (Audeh M. W., et al., Lancet (2010); 376 (9737): 245-51).

DNA damage repair processes are critical for genome maintenance and stability, among which, double strand breaks (DSBs) are predominantly repaired by the nonhomologous end joining (NHEJ) pathway in G1 phase of the cell cycle and by homologous recombination (HR) in S-G2 phases. A less addressed alternative end-joining (alt-EJ), also known as microhomology-mediated end-joining (MMEJ) pathway, is commonly considered as a “backup” DSB repair pathway when NHEJ or HR are compromised. Numerous genetic studies have highlighted a role for DNA polymerase theta (Polθ, encoded by POLQ) in stimulating MMEJ in higher organisms (Chan S. H., et al., PLOS Genet. (2010); 6: e1001005; Roerink S. F., et al., Genome research. (2014); 24: 954-962; Ceccaldi R., et. al., Nature (2015); 518: 258-62; and Mateos-Gomez P. A., et al., Nature (2015); 518: 254-57).

Polθ is distinct among human DNA polymerases, exhibiting not only a C-terminal DNA polymerase domain but also an N-terminal helicase domain separated by a long and lesser-conserved central domain of unknown function beyond Rad51 binding (Seki eta. Al, 2003, Shima et al 2003; Yousefzadeh and Wood 2013). The N-terminal ATPase/helicase domain belongs to the HELQ class of SF2 helicase super family. In homologous recombination deficient (HRD) cells, Polθ can carry out error-prone DNA synthesis at DNA damage sites through alt-EJ pathway. It has been shown that the helicase domain of Polθ causes suppression of HR pathway through disruption of Rad51 nucleoprotein complex formation involved in initiation of the HR-dependent DNA repair reactions following ionizing radiation. This anti-recombinase activity of Polθ promotes the alt-EJ pathway. In addition, the helicase domain of Polθ contributes to microhomology-mediated strand annealing (Chan S H et al., PLOS Genet. (2010); 6: e1001005; and Kawamura K et al., Int. J. Cancer (2004); 109: 9-16).

Polθ efficiently promotes end-joining in alt-EJ pathway by employing this annealing activity when ssDNA overhangs contain >2 bp of microhomology (Kent T., et al., Elife (2016); 5: e13740), and Kent T., et al., Nat. Struct. Mol. Biol. (2015); 22: 230-237). This reannealing activity is achieved through coupled actions of Rad51 interaction followed by ATPase-mediated displacement of Rad51 from DSB damage sites. Once annealed, the primer strand of DNA can be extended by the polymerase domain of Polθ.

The expression of Polθ is largely absent in normal cells but upregulated in breast, lung, and ovarian cancers (Ceccaldi R., et al., Nature (2015); 518, 258-62). Additionally, the increase of Polθ expression correlates with poor prognosis in breast cancer (Lemee F et al., Proc Natl Acad Sci USA. (2010); 107: 13390-5). It has been shown that cancer cells with deficiency in HR, NHEJ or ATM are highly dependent on Polθ expression (Ceccaldi R., et al., Nature (2015); 518: 258-62, Mateos-Gomez P A et al., Nature (2015); 518: 254-57, and Wyatt D. W., et al., Mol. Cell (2016); 63: 662-73). Therefore, Polθ is an attractive target for novel synthetic lethal therapy in cancers containing DNA repair defects.

### SUMMARY

Disclosed herein are certain thiadiazolyl derivatives that inhibit Polθ activity, in particular inhibit Polθ activity by inhibiting the ATP dependent helicase domain activity of Polθ. Also, disclosed are pharmaceutical compositions comprising such compounds and methods of treating and/or preventing diseases treatable by inhibition of Polθ such as cancer, including homologous recombination (HR) deficient cancers.

In one aspect, provided is a compound of Formula (I), or a pharmaceutically acceptable salt thereof:

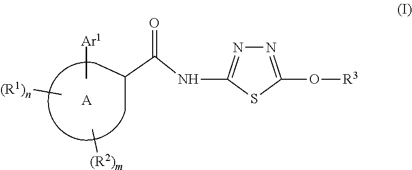

wherein ring A, Ar¹, R¹, R², R³, and subscripts n and m have the meanings provided hereinbelow.

In related aspects, provided are pharmaceutical compositions comprising a compound of Formula (I) or a subembodiment or a pharmaceutically acceptable salt thereof and at least one pharmaceutically acceptable excipient.

In another aspect, provided is a method for treating and/or preventing a disease characterized by overexpression of Polθ in a patient comprising administering to the patient a therapeutically effective amount of a compound of Formula (I) or a subembodiment or a pharmaceutically acceptable salt thereof. In one embodiment, the patient is in recognized need of such treatment. In another embodiment, the compound of Formula (I) or a subembodiment or a pharmaceutically acceptable salt thereof is administered in a pharmaceutical composition. In yet another embodiment, the disease is a cancer.

In still another aspect, provided is a method for treating and/or preventing a homologous recombinant (HR) deficient cancer in a patient comprising administering to the patient a therapeutically effective amount of a compound of Formula (I) or a subembodiment or a pharmaceutically acceptable salt thereof. In one embodiment, the patient is in recognized need of such treatment. In another embodiment, the compound of Formula (I) or a subembodiment or a pharmaceutically acceptable salt thereof is administered in a pharmaceutical composition.

In another aspect, provided is a method for inhibiting DNA repair by Polθ in a cancer cell comprising contacting the cell with an effective amount of a compound of Formula (I) or a subembodiment or a pharmaceutically acceptable salt thereof. In one embodiment, the cancer is HR deficient cancer.

In yet another aspect, provided is a method for treating and/or preventing a cancer in a patient, wherein the cancer is characterized by a reduction or absence of BRCA gene expression, the absence of the BRCA gene, or reduced function of BRCA protein, comprising administering to the subject a therapeutically effective amount of a compound of Formula (I) or a subembodiment or a pharmaceutically acceptable salt thereof, optionally in a pharmaceutical composition.

In still another aspect, provided is a compound of Formula (I) or a subembodiment or a pharmaceutically acceptable salt thereof for use in a method of treatment.

In still another aspect, provided is a compound of Formula (I) or a subembodiment or a pharmaceutically acceptable salt thereof for inhibiting DNA repair by Polθ in a cell. In one embodiment, the cell is HR deficient cell.

In another aspect, provided is a compound of Formula (I) or a subembodiment or a pharmaceutically acceptable salt thereof for use in the treatment and/or prevention of a disease in a patient, wherein the disease is characterized by overexpression of Polθ.

In yet another aspect, provided is a compound of Formula (I) or a subembodiment or a pharmaceutically acceptable salt thereof for use in the treatment and/or prevention of a cancer in a patient, wherein the cancer is characterized by a reduction or absence of BRCA gene expression, the absence of the BRCA gene, or reduced function of BRCA protein.

In still another aspect, provided is a compound of Formula (I) or a subembodiment or a pharmaceutically acceptable salt thereof for use in the treatment and/or prevention of a HR deficient cancer in a patient.

In another aspect, provided is a compound of Formula (I) or a subembodiment or a pharmaceutically acceptable salt thereof for use in the treatment and/or prevention of a cancer that is resistant to poly(ADP-ribose) polymerase (PARP) inhibitor therapy in a patient. Examples of cancers resistant to PARP-inhibitors include, but are not limited to, breast cancer, ovarian cancer, lung cancer, bladder cancer, liver cancer, head and neck cancer, pancreatic cancer, gastrointestinal cancer, and colorectal cancer.

In still another aspect, provided is the use of a compound of Formula (I) or a subembodiment or a pharmaceutically acceptable salt thereof in the manufacture of a medicament for the treatment or prevention of cancer, wherein the cancer is characterized by a reduction or absence of BRCA gene expression, the absence of the BRCA gene, or reduced function of BRCA protein.

In still another aspect, provided is the use of a compound of Formula (I) or a subembodiment or a pharmaceutically acceptable salt thereof in the manufacture of a medicament for the treatment or prevention of a HR deficient cancer.

In still another aspect, provided is the use of a compound of Formula (I) or a subembodiment or a pharmaceutically acceptable salt thereof in the manufacture of a medicament for the treatment of cancer that is resistant to poly(ADP-ribose) polymerase (PARP) inhibitor therapy in a patient.

In related aspects for the methods, uses and compositions above, the cancer is lymphoma, rhabdoid tumor, multiple myeloma, uterine cancer, gastric cancer, peripheral nervous system cancer, rhabdomyosarcoma, bone cancer, colorectal cancer, mesothelioma, breast cancer, ovarian cancer, lung cancer, fibroblast cancer, central nervous system cancer, urinary tract cancer, upper aerodigestive cancer, leukemia, kidney cancer, skin cancer, esophageal cancer, and pancreatic cancer (data from large scale drop out screens in cancer cell lines indicate that some cell lines from the above cancers are dependent on polymerase theta for proliferation https://depmap.org/portal/).

In some embodiments, a HR-deficient cancer is breast cancer. Breast cancer includes, but is not limited to, lobular carcinoma in situ (LCIS), a ductal carcinoma in situ (DCIS), an invasive ductal carcinoma (IDC), inflammatory breast cancer. Paget disease of the nipple. Phyllodes tumor. Angiosarcoma, adenoid cystic carcinoma, low-grade adenosquamous carcinoma, medullary carcinoma, mucinous carcinoma, papillary carcinoma, tubular carcinoma, metaplastic carcinoma, micropapillary carcinoma, mixed carcinoma, or another breast cancer, including but not limited to triple negative, HER positive, estrogen receptor positive, progesterone receptor positive. HER and estrogen receptor positive. HER and progesterone receptor positive, estrogen and progesterone receptor positive, and HER and estrogen and progesterone receptor positive. In other embodiments, HR-deficient cancer is ovarian cancer. Ovarian cancer includes, but is not limited to, epithelial ovarian carcinomas (EOC), maturing teratomas, dysgerminomas, endodermal sinus tumors, granulosa-theca tumors. Sertoli-Leydig cell tumors, and primary peritoneal arcinoma.

### DETAILED DESCRIPTION

Before the present invention is further described, it is to be understood that the invention is not limited to the particular embodiments set forth herein, and it is also to be understood that the terminology used herein is for the purpose of describing particular embodiments only, and is not intended to be limiting.

The singular forms “a,” “an,” and “the” as used herein and in the appended claims include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

When needed, any definition herein may be used in combination with any other definition to describe a composite structural group. By convention, the trailing element of any such definition is that which attaches to the parent moiety. For example, the composite group alkoxyalkyl means that an alkoxy group is attached to the parent molecule through an alkyl group.

The publications discussed herein are provided solely for their disclosure prior to the filing date of the present application. Further, the dates of publication provided may be different from the actual publication dates, which may need to be independently confirmed.

### Definitions

Unless otherwise stated, the following terms used in the specification and claims are defined for the purposes of this Application and have the following meaning:

The term “alkyl”, by itself or as part of another substituent, means, unless otherwise stated, a saturated straight or branched chain hydrocarbon radical, having the number of carbon atoms designated (i.e. C₁₋₈ means one to eight carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

The term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene, hexylene, and the like.

The term “alkoxy” refers to an alkyl group having an oxygen atom that connects the alkyl group to the point of attachment: alkyl-O—. As for an alkyl group, alkoxy groups can have any suitable number of carbon atoms, such as C₁₋₆, and can be straight or branched. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

As used herein, the term “cyano,” by itself or as part of another substituent, refers to a moiety having the formula —CN, i.e., a carbon atom triple-bonded to nitrogen atom.

The term “cycloalkyl” refers to a saturated or partially unsaturated hydrocarbon ring having the indicated number of ring atoms (e.g., C₃₋₆ cycloalkyl). Cycloalkyl can include any number of carbons, such as C₃₋₆, C₄₋₆, C₅₋₆, C₃₋₈, C₄₋₈, C₅₋₈, C₆₋₈, C₃₋₉, and C₃₋₁₀. Partially unsaturated cycloalkyl groups have one or more double or triple bonds in the ring, but cycloalkyl groups are not aromatic. Saturated monocyclic cycloalkyl rings include, for example, cyclopropyl, cyclobutyl, cyclopentyl, cyclohexyl, and cyclooctyl.

The term “cycloalkyloxy” refers to a cycloalkyl group having an oxygen atom that connects the cycloalkyl group to the point of attachment: cycloalkyl-O—. The cycloalkyl group is as defined herein.

The terms “spirocyclyl” or “spirocycloalkyl” refer to a saturated or partially unsaturated bicyclic ring having 6 to 12 ring atoms, where the two rings are connected via a single carbon atom (also called the spiroatom). Partially unsaturated spirocycloalkyl groups have one or more double or triple bonds in the ring, but spirocycloalkyl groups are not aromatic. Representative examples include, but are not limited to, spiro[3.3]heptane, spiro[4.4]nonane, spiro[3.4]octane, and the like.

The term “bridged cycloalkyl” means a monocyclic 6- to 11-membered hydrocarbon radical in which two non-adjacent ring atoms are linked by a (CH₂)ₙ group where n is 1 to 3 (also referred to herein as the bridging group). Examples of bridged cycloalkyl include but are not limited to bicyclo[2.2.1]heptane and bicyclo[2.2.2]octane. For brevity, the term is also meant to include bridged polycyclic hydrocarbon groups such as adamantane.

The term “heterocycloalkyl” refers to a saturated or partially unsaturated monocyclic ring having the indicated number of ring vertices (e.g., a 3- to 7-membered ring) and having from one to five heteroatoms selected from N, O, and S as ring vertices. Partially unsaturated heterocycloalkyl groups have one or more double or triple bonds in the ring, but heterocycloalkyl group are not aromatic. Heterocycloalkyl groups can include any number of ring atoms, such as, 3 to 6, 4 to 6, 5 to 6, 3 to 7, 4 to 7, or 5 to 7 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. Non-limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. A heterocycloalkyl group can be attached to the remainder of the molecule through a ring carbon or a heteroatom.

The term “bicyclic heterocycloalkyl” or “bicyclic heterocyclyl” refers to a saturated or partially unsaturated fused bicyclic ring having the indicated number of ring vertices (e.g., a 6- to 12-membered ring) and having from one to five heteroatoms selected from N, O, and S as ring vertices. Partially unsaturated bicyclic heterocycloalkyl groups have one or more double or triple bonds in the ring, but bicyclic heterocycloalkyl groups are not aromatic. Bicyclic heterocycloalkyl groups can include any number of ring atoms, such as, 6 to 8, 6 to 9, 6 to 10, 6 to 11, or 6 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. Non-limiting examples of bicyclic heterocycloalkyl groups include decahydro-1,5-naphthyridine, octahydropyrrolo[1,2-a]pyrazine, and the like.

The terms “bridged heterocyclyl” or “bridged heterocycloalkyl” refers to a heterocycloalkyl ring (having 5 to 8 ring vertices) in which two non-adjacent ring atoms are linked by a (CRR′)ₙ group where n is 1 to 3 and each R is independently H or methyl (also may be referred to herein as “bridging” group). Bridged heterocyclyl groups have one to five heteroatoms selected from N, O, and S as ring vertices. The heteroatom ring vertices can be in both the heterocycloalkyl ring portion as well as the bridging group. When in the bridging group, the heteroatom replaces a CRR′ group. Examples include, but are not limited to, 2-azabicyclo[2.2.2]octane, quinuclidine, 7-oxabicyclo[2.2.1]heptane, and the like.

The terms “spiroheterocyclyl” or “spiroheterocycloalkyl” refer to a saturated or partially unsaturated bicyclic ring having 6 to 12 ring atoms, where the two rings are connected via a single carbon atom (also called the spiroatom). Spiroheterocyclyl groups have from one to five heteroatoms selected from N, O, and S as ring vertices, and the nitrogen atom(s) are optionally quaternized. Partially unsaturated spiroheterocycloalkyl groups have one or more double or triple bonds in the ring, but spiroheterocycloalkyl groups are not aromatic. Representative examples include, but are not limited to, 4-oxaspiro[2.4]heptane, 2,6-diazaspiro[3.3]heptane, 2,6-diazaspiro[3.4]octane, 2-azaspiro[3.4]octane, 2-azaspiro[3.5]-nonane, 2,7-diazaspiro[4.4]nonane, and the like.

The terms “halo” or “halogen,” by themselves or as part of another substituent, mean, unless otherwise stated, a fluorine, chlorine, bromine, or iodine atom.

The term “haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, the term “C₁₋₄ haloalkyl” is meant to include trifluoromethyl, 2,2,2-trifluoroethyl, 4-chlorobutyl, 3-bromopropyl, and the like.

The term “haloalkoxy” refers to an alkoxy group where some or all of the hydrogen atoms are substituted with halogen atoms. As for an alkyl group, haloalkoxy groups can have any suitable number of carbon atoms, such as C₁₋₆, and can be straight or branched, and are substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

The term “hydroxyalkyl” refers to an alkyl group where one of the hydrogen atoms is substituted with a hydroxy (—OH) group. As for an alkyl group, hydroxyalkyl groups can have any suitable number of carbon atoms, such as C₁₋₆, and can be straight or branched. Hydroxyalkyl groups include, for example, hydroxymethyl, 1-hydroxylethyl, 2-hydroxyethyl, 2-hydroxylpropan-2-yl, etc.

The term “aryl” means, unless otherwise stated, a polyunsaturated, typically aromatic, hydrocarbon group which can be a single ring or multiple rings (up to three rings) which are fused together or linked covalently. Non-limiting examples of aryl groups include phenyl, naphthyl and biphenyl.

The term “heteroaryl” refers to a 5- to 10-membered aromatic ring (or fused ring system) that contains from one to five heteroatoms selected from N, O, and S. Heteroaryl groups can include any number of ring atoms, such as, 5 to 6, 5 to 8, 6 to 8, 6 to 9, 9 to 10, 9, 10 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. A heteroaryl group can be attached to the remainder of the molecule through a heteroatom. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

As used herein, the term “heteroatom” is meant to include oxygen (O), nitrogen (N), sulfur (S).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain compounds of the present disclosure can exist in unsolvated forms as well as solvated forms, including hydrated forms. For the purposes of the present disclosure, the solvated forms are equivalent to unsolvated forms and are intended to be encompassed within the scope of the present invention. Certain compounds of the present invention may exist in multiple crystalline or amorphous forms. All potential physical forms are intended to be within the scope of the present invention.

Certain compounds of the present invention possess asymmetric carbon atoms (optical centers); the racemates, diastereomers, and individual isomers (e.g., separate enantiomers) are all intended to be encompassed within the scope of the present invention. When a stereochemical depiction is shown, it is meant to refer the compound in which one of the isomers is present and substantially free of the other isomer. “Substantially free of” another isomer indicates at least an 80/20 ratio of the two isomers, more preferably 90/10, or 95/5 or more. In some embodiments, one of the isomers will be present in an amount of at least 99%.

The compounds of the present invention may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants of the compounds of the invention can have altered pharmacokinetic and pharmacodynamic characteristics which can contribute to enhanced safety, tolerability or efficacy during treatment. All isotopic variations of the compounds of the present invention, whether radioactive or not, are intended to be encompassed within the scope of the present invention.

The terms “administration,” “administer” and the like, as they apply to, for example, a subject, cell, tissue, organ, or biological fluid, refer to contact of, for example, an Polθ modulator, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat.” “treating.” “treatment” and the like refer to a course of action (such as administering a Polθ modulator or a pharmaceutical composition comprising same) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease.

The term “in need of treatment” as used herein refers to a judgment made by a physician or other caregiver that a subject requires or will benefit from treatment. This judgment is made based on a variety of factors that are in the realm of the physician's or caregiver's expertise. For example, the patient has been diagnosed as having a disease linked to overexpression of Polθ or a homologous recombination (HR)-deficient cancer.

The terms “prevent,” “preventing.” “prevention” and the like refer to a course of action (such as administering an Polθ modulator or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of an Polθ modulator (or, e.g., a metabolite thereof) at a particular time post-administration may be indicative of whether a therapeutically effective amount has been used.

The terms “modulate,” “modulation” and the like refer to the ability of a molecule (e.g., an activator or an inhibitor) to increase or decrease the function or activity of Polθ, either directly or indirectly. A modulator may act alone, or it may use a cofactor, e.g., a protein, metal ion, or small molecule. Examples of modulators include small molecule compounds and other bioorganic molecules.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term “proliferative activity” encompasses an activity that promotes, that is necessary for, or that is specifically associated with, for example, normal cell division, as well as cancer, tumors, dysplasia, cell transformation, metastasis, and angiogenesis.

Certain compounds of the present disclosure can exist as tautomers and/or geometric isomers. All possible tautomers and cis and trans isomers, as individual forms and mixtures thereof are within the scope of this disclosure. For example, certain hydroxy substituted compounds may exist as tautomers as shown below:

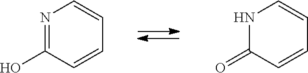 [SMILES: Oc1ccccn1 ; O=c1cccc[nH]1]

“Pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

As used herein, a wavy line, “ [symbol] ”, that intersects a single, double or triple bond in any chemical structure depicted herein, represent the point attachment of the single, double, or triple bond to the remainder of the molecule. Additionally, a bond extending to the center of a ring (e.g., a phenyl ring) is meant to indicate attachment at any of the available ring vertices. One of skill in the art will understand that multiple substituents shown as being attached to a ring will occupy ring vertices that provide stable compounds and are otherwise sterically compatible.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that the numerical value encompasses ±10%, preferably ±5% of the recited numerical value.

“Disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Patient” is generally synonymous with the term “subject” and as used herein includes all mammals including humans. Examples of patients include humans, livestock such as cows, goats, sheep, pigs, and rabbits, and companion animals such as dogs, cats, rabbits, and horses. Preferably, the patient is a human.

“Inhibiting”, “reducing,” or any variation of these terms in relation of Polθ, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of Polθ activity compared to its normal activity.

The term “homologous recombination” refers to the cellular process of genetic recombination in which nucleotide sequences are exchanged between two similar or identical DNA.

The term “homologous recombination (HR) deficient cancer” refers to a cancer that is characterized by a reduction or absence of a functional HR repair pathway. HR deficiency may arise from absence of one or more HR-associated genes or presence of one or more mutations in one or more HR-associated genes. Examples of HR-associated genes include BRCA1, BRCA2, RAD54, RAD51B, Ct1P (Choline Transporter-Like Protein), PALB2 (Partner and Localizer of BRCA2), XRCC2 (X-ray repair complementing defective repair in Chinese hamster cells 2), RECQL4 (RecQ Protein-Like 4), BLM (Bloom syndrome, RecQ helicase-like), WRN (Werner syndrome, one or more HR-associated genes) Nbs 1 (Nibrin), and genes encoding Fanconi anemia (FA) proteins or FA-like genes e.g, FANCA, FANCB, FANCC, FANCD1 (BRCA2), FANCD2, FANCE, FANCF, FANCG, FANCI, FANJ (BRIP1), FANCL, FANCM, FANCN (RALB2), FANCP (SLX4), FANCS (BRCA1), RAD51C, and XPF.

The term “Polθ overexpression” refers to the increased expression or activity of Polθ in a diseases cell e.g., cancerous cell, relative to expression or activity of Polθ in a normal cell (e.g., non-diseased cell of the same kind). The amount of Polθ can be at least 2-fold, at least 3-fold, at least 4-fold, at least 5-fold, at least 10-fold, or more relative to the Polθ expression in a normal cell. Examples of Polθ cancers include, but are not limited to, breast, ovarian, cervical, lung, colorectal, gastric, bladder and prostate cancers.

### Compounds

In some aspects, provided herein are compounds of Formula (I)

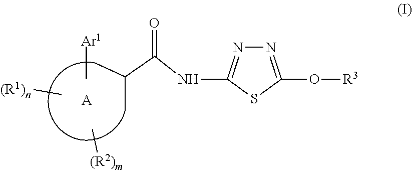

wherein:
ring A is selected from the group consisting of phenyl and a 5- to 6-membered heteroaryl ring having 1 to 4 heteroatoms independently selected from N, O, and S;
the subscripts m and n are each independently 0 or 1;
R¹ is selected from the group consisting of C₁₋₆ alkyl, halo, C₁₋₆ haloalkyl, —X¹—O—C₁₋₆ alkyl, C₁₋₆ haloalkoxy, —X¹ cyano, —NO₂, —C(O)OR^{a}, —NR^{a}C(O)R^{b}, —X¹—C(O)NR^{a}R^{b}, —X¹—OH, C₃₋₆ cycloalkyl, —X¹—O—C₃₋₆ cycloalkyl, C₁₋₆ hydroxyalkynyl, —X¹—NR^{a}R^{b}, —X¹—S(O)₂R^{a}, —X¹—S(O)₂NR^{a}R^{b}, X¹—X^{1a}—OR^{a}, and a 4- to 6-member heterocycloalkyl having 1 to 4 heteroatoms as ring vertices independently selected from N, O, and S;
R² is selected from the group consisting of C₁₋₆ alkyl, halo, C₁₋₆ haloalkyl, C₁₋₆ haloalkoxy, and
   —X¹-cyano, wherein
   each X¹ is independently selected from a bond and C₁₋₄ alkylene,
   X^{1a} is 3- to 6-membered heterocycloalkylene having 1 to 4 heteroatoms as ring vertices independently selected from N, O, and S, and
   R^{a} and R^{b} are independently selected from the group consisting of hydrogen, C₁₋₆ alkyl, and C₁₋₆ haloalkyl; and
Ar¹ is selected form the group consisting of phenyl, naphthyl, pyridin-2-one, and a 5- to 10-membered heteroaryl ring having 1 to 4 heteroatoms independently selected from N, O, and S, wherein Ar¹ is substituted with 0 to 4 R^{1a} substituents;
each R^{1a} is independently selected from C₁₋₆ alkyl, halo, C₁₋₆ haloalkyl, —X²—O—C₁₋₆ alkyl, C₁₋₆ haloalkoxy, C₃₋₆ cycloalkyl, C₃₋₆ cycloalkyloxy, —C(O)R^{c}, —C(O)₂R^{c}, —NR^{c}C(O)R^{d}, —O—C₁₋₄ alkylene-O—C₁₋₄ alkyl, —X²—C(O)NR^{c}R^{d}, —X²—S(O)₂NR^{c}R^{d}, —X²—NR^{c}R^{d}, —C(O)NR^{c}R^{d}, —X²-cyano, —O—X²-cyano, —X²—S(O)R^{c}, —X²—S(O)₂R^{c}, —X²—N(R^{d})S(O)₂R^{c}, —P(O)R^{c}R^{d}, —Y and —X²—OH; or
two R^{1a} groups on adjacent ring vertices combine to form a 4 to 6 membered cycloalkyl or heterocycloalkyl, having 0 to 2 heteroatoms as ring vertices independently selected from N, O, and S, and which is substituted with 0 to 4 groups independently selected from oxo, halo, C₁₋₄ alkyl, C₁₋₄ alkoxy, and C₁₋₄ haloalkyl;
   wherein
   each Y is independently selected from phenyl, benzyl, 4- to 6-membered heterocycloalkyl, and 5- or 6-membered heteroaryl, wherein each heterocycloalkyl and heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl;
   each X² is independently selected from a bond and C₁₋₄ alkylene; and
   each R^{c} and R^{d} are independently selected from the group consisting of hydrogen, C₁₋₆ alkyl, C₃₋₅ cycloalkyl and C₁₋₆ haloalkyl; and
R³ is a member selected from the group consisting of
   (i) C₃₋₆ cycloalkyl, C₆₋₁₁ bridged cycloalkyl and C₆₋₁₂ spirocycloalkyl;
   (ii) 3- to 6-membered heterocycloalkyl having 1 to 4 heteroatoms as ring vertices independently selected from N, O, and S;
   (iii) 6- to 10-membered bicyclic heterocyclyl having 1 to 4 heteroatoms as ring vertices independently selected from N, O, and S;
   (iv) 6- to 10-membered bridged heterocyclyl having 1 to 4 heteroatoms as ring vertices independently selected from N, O, and S;
   (v) 6- to 12-membered spiroheterocyclyl having 1 to 4 heteroatoms as ring vertices independently selected from N, O, and S;
   (vi) hydrogen;
   (vii) C₁₋₆ alkyl, or C₂₋₆ alkynyl,
wherein
each R³ member of (i) through (v) is substituted with 0 to 4 R^{3a} substituents, each of which is independently selected from C₁₋₆ alkyl, C₂₋₆ alkenyl, C₂₋₆ alkynyl, halo, C₁₋₆ haloalkyl, C₁₋₆ haloalkoxy, —X³—O—C₁₋₆ alkyl, —X³—OH, —NR^{e}R^{f}, —ONO₂, 4- to 6-member heterocycloalkyl having 1 to 4 heteroatoms as ring vertices independently selected from N, O, and S, —NR^{e}C(O)R^{f}, —X³—NR^{e}R^{f}, —X³-cyano, and oxo; and
R³ member (vii) is substituted with from 0 to 3 R^{3b} substituents selected from the group consisting of halo, C₁₋₃ haloalkyl, C₁₋₆ haloalkoxy, —O—C₁₋₆ alkyl, cyano, —OH, —NR^{e}R^{f},
   —CONR^{e}R^{f}, and oxo, wherein
   each X³ is independently selected from a bond and C₁₋₄ alkylene, and
   each R^{e} and R^{f} is independently selected from H, C₁₋₆ alkyl, C₁₋₆ haloalkyl, C₃₋₆ cycloalkyl and —C₁₋₃alkylene-C₃₋₆ cycloalkyl;
or a pharmaceutically acceptable salt thereof.

In some aspects, provided herein are compounds of Formula (I):

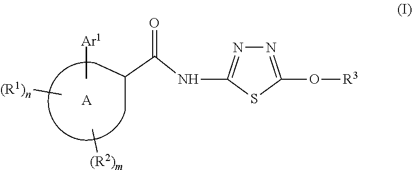

wherein:
ring A is selected from the group consisting of phenyl and a 5- to 10-membered heteroaryl ring having 1 to 4 heteroatoms independently selected from N, O, and S;
the subscripts m and n are each independently 0 or 1;
R¹ is selected from the group consisting of C₁₋₆ alkyl, halo, C₁₋₆ haloalkyl, —X¹—O—C₁₋₆ alkyl,
   C₁₋₆ haloalkoxy, —X¹-cyano, —NO₂, —C(O)OR^{a}, —NR^{a}C(O)R^{b}, —X¹—C(O)NR^{a}R^{b}, —X¹—OH, C₃₋₆ cycloalkyl, —X¹—O—C₃₋₆ cycloalkyl, C₁₋₆ hydroxyalkynyl, —X¹—NR^{a}R^{b}, —X¹—S(O)₂R^{a}, —X¹—S(O)₂NR^{a}R^{b}, X¹—X^{1a}—OR^{a}, and a 4- to 6-member heterocycloalkyl having 1 to 4 heteroatoms as ring vertices independently selected from N, O, and S;
R² is selected from the group consisting of C₁₋₆ alkyl, halo, C₁₋₆ haloalkyl, C₁₋₆ haloalkoxy, and
   —X¹-cyano, wherein
   each X¹ is independently selected from a bond and C₁₋₄ alkylene,
   X^{1a} is 3- to 6-membered heterocycloalkylene having 1 to 4 heteroatoms as ring vertices independently selected from N, O, and S, and
   R^{a} and R^{b} are independently selected from the group consisting of hydrogen, C₁₋₆ alkyl, and C₁₋₆ haloalkyl; and
Ar¹ is selected form the group consisting of phenyl, naphthyl, pyridin-2-one, and a 5- to 10-membered heteroaryl ring having 1 to 4 heteroatoms independently selected from N, O, and S, wherein Ar¹ is substituted with 0 to 4 R^{1a} substituents;
each R^{1a} is independently selected from C₁₋₆ alkyl, halo, C₁₋₆ haloalkyl, —X²—O—C₁₋₆ alkyl, C₁₋₆ haloalkoxy, C₃₋₆ cycloalkyl, C₃₋₆ cycloalkyloxy, —C(O)R^{c}, —C(O)₂R^{c}, —NR^{c}C(O)R^{d}, —O—C₁₋₄ alkylene-O—C₁₋₄ alkyl, —X²—C(O)NR^{c}R^{d}, —X²—S(O)₂NR^{c}R^{d}, —X²—NR^{c}R^{d}, —C(O)NR^{c}R^{d}, —X²-cyano, —O—X²-cyano, —X²—S(O)R^{c}, —X²—S(O)₂R^{c}, —X²—N(R^{d})S(O)₂R^{c}, —P(O)R^{c}R^{d}, —Y and —X²—OH; or
two R^{1a} groups on adjacent ring vertices combine to form a 4 to 6 membered cycloalkyl or heterocycloalkyl, having 0 to 2 heteroatoms as ring vertices independently selected from N, O, and S, and which is substituted with 0 to 4 groups independently selected from oxo, halo, C₁₋₄ alkyl, C₁₋₄ alkoxy, and C₁₋₄ haloalkyl;
   wherein
   each Y is independently selected from phenyl, benzyl, 4- to 6-membered heterocycloalkyl, and 5- or 6-membered heteroaryl, wherein each heterocycloalkyl and heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl;
   each X² is independently selected from a bond and C₁₋₄ alkylene; and
   each R^{c} and R^{d} are independently selected from the group consisting of hydrogen, C₁₋₆ alkyl, C₃₋₅ cycloalkyl and C₁₋₆ haloalkyl; and
R³ is a member selected from the group consisting of
   (i) C₃₋₆ cycloalkyl, C₆₋₁₁ bridged cycloalkyl and C₆₋₁₂ spirocycloalkyl;
   (ii) 3- to 6-membered heterocycloalkyl having 1 to 4 heteroatoms as ring vertices independently selected from N, O, and S;
   (iii) 6- to 10-membered bicyclic heterocyclyl having 1 to 4 heteroatoms as ring vertices independently selected from N, O, and S;
   (iv) 6- to 10-membered bridged heterocyclyl having 1 to 4 heteroatoms as ring vertices independently selected from N, O, and S;
   (v) 6- to 12-membered spiroheterocyclyl having 1 to 4 heteroatoms as ring vertices independently selected from N, O, and S;
   (vi) hydrogen;
   (vii) C₁₋₆ alkyl, or C₂₋₆ alkynyl,
wherein
each R³ member of (i) through (v) is substituted with 0 to 4 R^{3a} substituents, each of which is independently selected from C₁₋₆ alkyl, C₂₋₆ alkenyl, C₂₋₆ alkynyl, halo, C₁₋₆ haloalkyl, C₁₋₆ haloalkoxy, —X³—O—C₁₋₆ alkyl, —X³—OH, —NR^{e}R^{f}, —ONO₂, 4- to 6-member heterocycloalkyl having 1 to 4 heteroatoms as ring vertices independently selected from N, O, and S, —NR^{e}C(O)R^{f}, —X³—NR^{e}R^{f}, —X³-cyano, and oxo; and
R³ member (vii) is substituted with from 0 to 3 R^{3b} substituents selected from the group consisting of halo, C₁₋₃ haloalkyl, C₁₋₆ haloalkoxy, —O—C₁₋₆ alkyl, cyano, —OH, —NR^{e}R^{f},
   —CONR^{e}R^{f}, and oxo, wherein
   each X³ is independently selected from a bond and C₁₋₄ alkylene, and
   each R^{e} and R^{f} is independently selected from H, C₁₋₆ alkyl, C₁₋₆ haloalkyl, C₃₋₆ cycloalkyl and —C₁₋₃alkylene-C₃₋₆ cycloalkyl;
or a pharmaceutically acceptable salt thereof;
provided that the compound of Formula I is other than

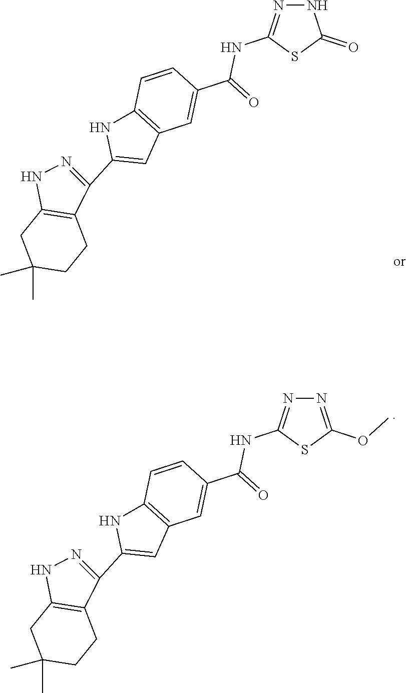

In some aspects, provided herein are compounds of Formula (I)

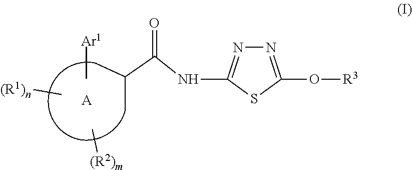

wherein:
ring A is selected from the group consisting of phenyl and a 5- to 10-membered heteroaryl ring having 1 to 4 heteroatoms independently selected from N, O, and S;
the subscripts m and n are each independently 0 or 1;
R¹ is selected from the group consisting of C₁₋₆ alkyl, halo, C₁₋₆ haloalkyl, —X¹—O—C₁₋₆ alkyl, C₁₋₆ haloalkoxy, —X¹-cyano, —NO₂, —C(O)OR^{a}, —NR^{a}C(O)R^{b}, —X¹—C(O)NR^{a}R^{b}, —X¹—OH, C₃₋₆ cycloalkyl, —X¹—O—C₃₋₆ cycloalkyl, C₁₋₆ hydroxyalkynyl, —X¹—NR^{a}R^{b}, —X¹—S(O)₂R^{a}, —X¹—S(O)₂NR^{a}R^{b}, X¹—X^{1a}—OR^{a}, and a 4- to 6-member heterocycloalkyl having 1 to 4 heteroatoms as ring vertices independently selected from N, O, and S;
R² is selected from the group consisting of C₁₋₆ alkyl, halo, C₁₋₆ haloalkyl, C₁₋₆ haloalkoxy, and
   —X¹-cyano, wherein
   each X¹ is independently selected from a bond and C₁₋₄ alkylene,
   X^{1a} is 3- to 6-membered heterocycloalkylene having 1 to 4 heteroatoms as ring vertices independently selected from N, O, and S, and
   R^{a} and R^{b} are independently selected from the group consisting of hydrogen, C₁₋₆ alkyl, and C₁₋₆ haloalkyl; and
Ar¹ is selected form the group consisting of phenyl, naphthyl, pyridin-2-one, and a 5- to 10-membered heteroaryl ring having 1 to 4 heteroatoms independently selected from N, O, and S, wherein Ar¹ is substituted with 0 to 4 R^{1a} substituents;
each R^{1a} is independently selected from C₁₋₆ alkyl, halo, C₁₋₆ haloalkyl, —X²—O—C₁₋₆ alkyl, C₁₋₆ haloalkoxy, C₃₋₆ cycloalkyl, C₃₋₆ cycloalkyloxy, —C(O)R^{c}, —C(O)₂R^{c}, —NR^{c}C(O)R^{d}, —O—C₁₋₄ alkylene-O—C₁₋₄ alkyl, —X²—C(O)NR^{c}R^{d}, —X²—S(O)₂NR^{c}R^{d}, —X²—NR^{c}R^{d}, —C(O)NR^{c}R^{d}, —X²-cyano, —O—X²-cyano, —X²—S(O)R^{c}, —X²—S(O)₂R^{c}, —X²—N(R^{d})S(O)₂R^{c}, —P(O)R^{c}R^{d}, —Y and —X²—OH; or
two R^{1a} groups on adjacent ring vertices combine to form a 4 to 6 membered cycloalkyl or heterocycloalkyl, having 0 to 2 heteroatoms as ring vertices independently selected from N, O, and S, and which is substituted with 0 to 4 groups independently selected from oxo, halo, C₁₋₄ alkyl, C₁₋₄ alkoxy, and C₁₋₄ haloalkyl;
   wherein
   each Y is independently selected from phenyl, benzyl, 4- to 6-membered heterocycloalkyl, and 5- or 6-membered heteroaryl, wherein each heterocycloalkyl and heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl;
   each X² is independently selected from a bond and C₁₋₄ alkylene; and
   each R^{c} and R^{d} are independently selected from the group consisting of hydrogen, C₁₋₆ alkyl, C₃₋₅ cycloalkyl and C₁₋₆ haloalkyl; and
R³ is a member selected from the group consisting of
   (i) C₃₋₆ cycloalkyl, C₆₋₁₁ bridged cycloalkyl and C₆₋₁₂ spirocycloalkyl;
   (ii) 3- to 6-membered heterocycloalkyl having 1 to 4 heteroatoms as ring vertices independently selected from N, O, and S;
   (iii) 6- to 10-membered bicyclic heterocyclyl having 1 to 4 heteroatoms as ring vertices independently selected from N, O, and S;
   (iv) 6- to 10-membered bridged heterocyclyl having 1 to 4 heteroatoms as ring vertices independently selected from N, O, and S;
   (v) 6- to 12-membered spiroheterocyclyl having 1 to 4 heteroatoms as ring vertices independently selected from N, O, and S;
   (vi) hydrogen;
   (vii) C₁₋₆ alkyl, or C₂₋₆ alkynyl,
wherein
each R³ member of (i) through (v) is substituted with 0 to 4 R^{3a} substituents, each of which is independently selected from C₁₋₆ alkyl, C₂₋₆ alkenyl, C₂₋₆ alkynyl, halo, C₁₋₆ haloalkyl, C₁₋₆ haloalkoxy, —X³—O—C₁₋₆ alkyl, —X³—OH, —NR^{e}R^{f}, —ONO₂, 4- to 6-member heterocycloalkyl having 1 to 4 heteroatoms as ring vertices independently selected from N, O, and S, —NR^{e}C(O)R^{f}, —X³—NR^{e}R^{f}, —X³-cyano, and oxo; and
R³ member (vii) is substituted with from 0 to 3 R^{3b} substituents selected from the group consisting of halo, C₁₋₃ haloalkyl, C₁₋₆ haloalkoxy, —O—C₁₋₆ alkyl, cyano, —OH, —NR^{e}R^{f},
   —CONR^{e}R^{f}, and oxo, wherein
   each X³ is independently selected from a bond and C₁₋₄ alkylene, and
   each R^{e} and R^{f} is independently selected from H, C₁₋₆ alkyl, C₁₋₆ haloalkyl, C₃₋₆ cycloalkyl and —C₁₋₃alkylene-C₃₋₆ cycloalkyl;
or a pharmaceutically acceptable salt thereof.

In some embodiments, the compounds of Formula I are other than

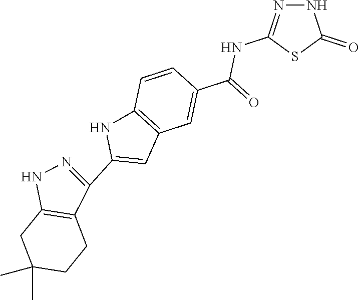 [SMILES: CC1(C)CCc2c(-c3cc4cc(C(=O)Nc5n[nH]c(=O)s5)ccc4[nH]3)n[nH]c2C1]

N-(4,5-dihydro-5-oxo-1,3,4-thiadiazol-2-yl)-2-(4,5,6,7-tetrahydro-6,6-dimethyl-1H-indazol-3-yl)-1H-Indole-5-carboxamide (CAS No. 1309787-94-7); or

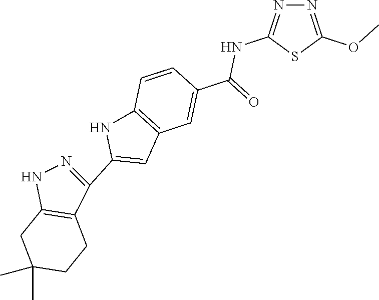

N-(5-methoxy-1,3,4-thiadiazol-2-yl)-2-(4,5,6,7-tetrahydro-6,6-dimethyl-1H-indazol-3-yl)-1H-Indole-5-carboxamide (CAS No. 1309795-15-0).

In some embodiments, the compounds of Formula I are other than those where ring A is

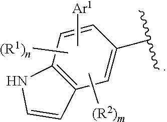

In some embodiments, the compounds of Formula I are other than those where ring A is indole.

In some aspects, provided herein are compounds of Formula (I)

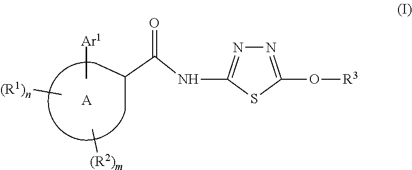

wherein:
ring A is selected from the group consisting of phenyl and a 5- to 10-membered heteroaryl ring having 1 to 4 heteroatoms independently selected from N, O, and S;
the subscripts m and n are each independently 0 or 1;
R¹ is selected from the group consisting of C₁₋₆ alkyl, halo, C₁₋₆ haloalkyl, —X¹—O—C₁₋₆ alkyl, C₁₋₆ haloalkoxy, —X¹-cyano, —C(O)OR^{a}, —NR^{a}C(O)R^{b}, —X¹—C(O)NR^{a}R^{b}, —X¹—OH, C₃₋₆ cycloalkyl, —X¹—O—C₃₋₆ cycloalkyl, C₁₋₆ hydroxyalkynyl, —X¹—NR^{a}R^{b}, —X¹—S(O)₂R^{a}, —X¹—S(O)₂NR^{a}R^{b}, and X¹—X^{1a}—OR^{a};
R² is selected from the group consisting of C₁₋₆ alkyl, halo, C₁₋₆ haloalkyl, C₁₋₆ haloalkoxy, and
   —X¹-cyano, wherein
   each X¹ is independently selected from a bond and C₁₋₄ alkylene,
   X^{1a} is 3- to 6-membered heterocycloalkylene having 1 to 4 heteroatoms as ring vertices independently selected from N, O, and S and
   R^{a} and R^{b} are independently selected from the group consisting of hydrogen, C₁₋₆ alkyl, and C₁₋₆ haloalkyl; and
Ar¹ is selected form the group consisting of phenyl and a 5- to 10-membered heteroaryl ring having 1 to 4 heteroatoms independently selected from N, O, and S, wherein Ar¹ is substituted with 0 to 4 R^{1a} substituents;
each R^{1a} is independently selected from C₁₋₆ alkyl, halo, C₁₋₆ haloalkyl, —X²—O—C₁₋₆ alkyl, C₁₋₆ haloalkoxy, C₃₋₆ cycloalkyl, C₃₋₆ cycloalkyloxy, —C(O)R^{c}, —NR^{c}C(O)R^{d}, —NR^{c}R^{d}, —X²—NR^{c}R^{d}, —C(O)NR^{c}R^{d}, —X²-cyano, —X²—S(O)R^{c} and —X²—OH; or
two R^{1a} groups on adjacent ring vertices combine to form a 4 to 6 membered heterocycloalkyl having 1 to 2 heteroatoms as ring vertices independently selected from N, O, and S, wherein
   each X² is independently selected from a bond and C₁₋₄ alkylene; and
   each R^{c} and R^{d} are independently selected from the group consisting of hydrogen, C₁₋₆ alkyl, and C₁₋₆ haloalkyl; and
R³ is a member selected from the group consisting of
   (i) C₃₋₆ cycloalkyl and C₆₋₁₂ spirocycloalkyl;
   (ii) 3- to 6-membered heterocycloalkyl having 1 to 4 heteroatoms as ring vertices independently selected from N, O, and S;
   (iii) 6- to 10-membered bicyclic heterocyclyl having 1 to 4 heteroatoms as ring vertices independently selected from N, O, and S;
   (iv) 6- to 10-membered bridged heterocyclyl having 1 to 4 heteroatoms as ring vertices independently selected from N, O, and S;
   (v) 6- to 12-membered spiroheterocyclyl having 1 to 4 heteroatoms as ring vertices independently selected from N, O, and S;
   (vi) hydrogen;
   (vii) C₁₋₆ alkyl, or C₂₋₆ alkynyl,
wherein
each R³ member of (i) through (v) is substituted with 0 to 4 R^{3a} substituents, each of which is independently selected from C₁₋₆ alkyl, C₂₋₆ alkenyl, C₂₋₆ alkynyl, halo, C₁₋₆ haloalkyl, C₁₋₆ haloalkoxy, —X³—O—C₁₋₆ alkyl, —X³—OH, —NR^{e}R^{f}.
   —NR^{e}C(O)R^{f}, —X³—NR^{e}R^{f}, —X³-cyano, and oxo; and
R³ member (vii) is substituted with from 0 to 3 R^{3b} substituents selected from the group consisting of halo, C₁₋₆ haloalkoxy, —O—C₁₋₆ alkyl, cyano, —OH, —NR^{e}R^{f}, and oxo, wherein
   each X³ is independently selected from a bond and C₁₋₄ alkylene, and
   each R^{e} and R^{f} is independently selected from H and C₁₋₆ alkyl;
or a pharmaceutically acceptable salt thereof.

In some aspects, provided herein are compounds of Formula (I)

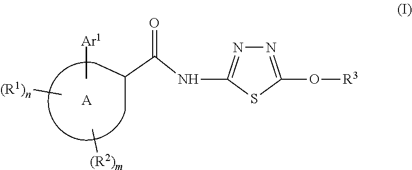

wherein:
ring A is selected from the group consisting of phenyl and a 5- to 10-membered heteroaryl ring having 1 to 4 heteroatoms independently selected from N, O, and S;
the subscripts m and n are each independently 0 or 1;
R¹ is selected from the group consisting of C₁₋₆ alkyl, halo, C₁₋₆ haloalkyl, —X¹—O—C₁₋₆ alkyl, C₁₋₆ haloalkoxy, —X¹-cyano, —C(O)OR^{a}, —NR^{a}C(O)R^{b}, —X¹—C(O)NR^{a}R^{b}, —X¹—OH, C₃₋₆ cycloalkyl, C₁₋₆ hydroxyalkynyl, —X¹—NR^{a}R^{b}, —X¹—S(O)₂R^{a}, and —X¹—S(O)₂NR^{a}R^{b};
R² is selected from the group consisting of C₁₋₆ alkyl, halo, C₁₋₆ haloalkyl, C₁₋₆ haloalkoxy, and —X¹-cyano, wherein
   each X¹ is independently selected from a bond and C₁₋₄ alkylene, and
   R^{a} and R^{b} are independently selected from the group consisting of hydrogen, C₁₋₆ alkyl, and C₁₋₆ haloalkyl; and
Ar¹ is selected form the group consisting of phenyl and a 5- to 10-membered heteroaryl ring having 1 to 4 heteroatoms independently selected from N, O, and S, wherein Ar¹ is substituted with 0 to 4 R^{1a} substituents;
each R^{1a} is independently selected from C₁₋₆ alkyl, halo, C₁₋₆ haloalkyl, —X²—O—C₁₋₆ alkyl, C₁₋₆ haloalkoxy, C₃₋₆ cycloalkyl, C₃₋₆ cycloalkyloxy, —C(O)R^{c}, —NR^{c}C(O)R^{d}, —NR^{c}R^{d}, —X²—NR^{c}R^{d}, —C(O)NR^{c}R^{d}, —X²-cyano, and —X²—OH, wherein
   each X² is independently selected from a bond and C₁₋₄ alkylene; and
   each R^{c} and R^{d} are independently selected from the group consisting of hydrogen, C₁₋₆ alkyl, and C₁₋₆ haloalkyl; and
R³ is a member selected from the group consisting of
   (i) C₃₋₆ cycloalkyl and C₆₋₁₂ spirocycloalkyl;
   (ii) 3- to 6-membered heterocycloalkyl having 1 to 4 heteroatoms as ring vertices independently selected from N, O, and S;
   (iii) 6- to 10-membered bicyclic heterocyclyl having 1 to 4 heteroatoms as ring vertices independently selected from N, O, and S;
   (iv) 6- to 10-membered bridged heterocyclyl having 1 to 4 heteroatoms as ring vertices independently selected from N, O, and S;
   (v) 6- to 12-membered spiroheterocyclyl having 1 to 4 heteroatoms as ring vertices independently selected from N, O, and S;
   (vi) hydrogen;
   (vii) C₁₋₆ alkyl, or C₂₋₆ alkynyl,
wherein
each R³ member of (i) through (v) is substituted with 0 to 4 R^{3a} substituents, each of which is independently selected from C₁₋₆ alkyl, C₂₋₆ alkenyl, C₂₋₆ alkynyl, halo, C₁₋₆ haloalkyl, C₁₋₆ haloalkoxy, —X³—O—C₁₋₆ alkyl, —X³—OH, —NR^{e}R^{f}, —NR^{e}C(O)R^{f}, —X³—NR^{e}R^{f}, —X³-cyano, and oxo; and
R³ member (vii) is substituted with from 0 to 3 R^{3b} substituents selected from the group consisting of halo, C₁₋₆ haloalkoxy, —O—C₁₋₆ alkyl, cyano, —OH, —NR^{e}R^{f}, and oxo, wherein
   each X³ is independently selected from a bond and C₁₋₄ alkylene, and
   each R^{e} and R^{f} is independently selected from H and C₁₋₆ alkyl;
or a pharmaceutically acceptable salt thereof.

In some embodiments, ring A in Formula (I) is phenyl, pyridinyl, pyridazinyl, imidazolyl, pyrazolyl, triazolyl, imidazo[1,2-a]pyridinyl, [1,2,3]triazolo[1,5-a]pyridinyl, imidazo[1,5-a]pyridinyl, pyrrolo[2,3-b]pyridinyl, pyrrolo[3,2-b]pyridinyl, pyrazolo[1,5-a]pyridinyl, [1,2,4]triazolo[1,5-a]pyridinyl, 1,6-naphthyridinyl, or 1,7-naphthyridinyl.

In some embodiments, ring A in Formula (I) is a nine or ten membered heteroaryl ring. In some embodiments, ring A in Formula (I) is a nine membered heteroaryl ring. In some embodiments, ring A in Formula (I) is a ten membered heteroaryl ring.

In some embodiments, ring A in Formula (I) is imidazo[1,2-a]pyridinyl, [1,2,3]triazolo[1,5-a]pyridinyl, imidazo[1,5-a]pyridinyl, pyrrolo[2,3-b]pyridinyl, pyrrolo[3,2-b]pyridinyl, pyrazolo[1,5-a]pyridinyl, [1,2,4]triazolo[1,5-a]pyridinyl, 1,6-naphthyridinyl, or 1,7-naphthyridinyl.

In some embodiments, ring A in Formula (I) is a five or six membered heteroaryl ring. In some embodiments, ring A in Formula (I) is a five membered heteroaryl ring. In some embodiments, ring A in Formula (I) is a six membered heteroaryl ring.

In some embodiments, ring A in Formula (I) is pyridinyl, pyridazinyl, pyrimidinyl, imidazolyl, pyrazolyl, or triazolyl.

In some embodiments, ring A together with Ar¹, R¹, and R² in Formula (I) is selected from the group consisting of:

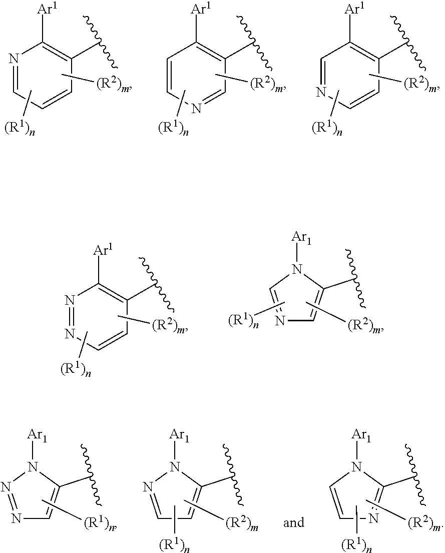

In some embodiments, ring A together with Ar¹, R¹, and R² in Formula (I) is selected from the group consisting of:

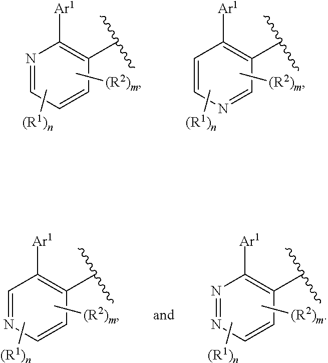

In some embodiments, ring A together with Ar¹, R¹, and R² in Formula (I) is selected from the group consisting of:

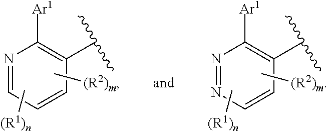

In some embodiments, ring A together with Ar¹, R¹, and R² in Formula (I) is selected from the group consisting of:

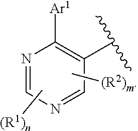

In some embodiments, ring A together with Ar¹ and R¹ in Formula (I) is:

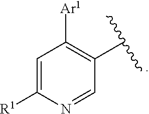

In some embodiments, ring A together with Ar¹ in Formula (I) is:

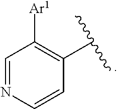

In some embodiments, ring A together with Ar¹ in Formula (I) is:

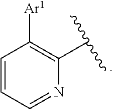

In some embodiments, ring A together with Ar¹ in Formula (I) is:

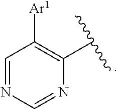

In some embodiments, ring A together with Ar¹ in Formula (I) is:

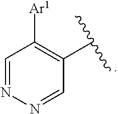

In some embodiments, ring A in Formula (I) is not pyrimidine.

In some embodiments, ring A in Formula (I) is selected from the group consisting of phenyl, pyridinyl, pyrimidinyl, and imidazo[1,2-a]pyridinyl, 1,2,3-triazole, pyrazolyl, isoxazolyl, imidazo[1,5-a]pyridinyl

In some embodiments, ring A in Formula (I) is selected from the group consisting of phenyl, pyridinyl, pyrimidinyl, and imidazo[1,2-a]pyridinyl.

In some embodiments, ring A in Formula (I) is phenyl.

In some embodiments, ring A in Formula (I) is pyridinyl.

In some embodiments, Ar¹ is attached to ring A in the ortho position relative to the amide substituent.

In some embodiments, when ring A is pyridyl, the N atom of pyridyl is oxidized to form the N-oxide.

In some embodiments, n in Formula (I) and subembodiments thereof is 1. In some embodiments, n in Formula (I) and subembodiments thereof is 0.

In some embodiments, n is 1 in Formula (I) and subembodiments thereof n is 1 and R¹ is a 4- to 6-member heterocycloalkyl having 1 to 4 heteroatoms as ring vertices independently selected from N, O, and S. In some embodiments, n is 1 in Formula (I) and subembodiments thereof n is 1 and R¹ is a 6-membered heterocycloalkyl having 1 to 4 heteroatoms as ring vertices independently selected from N, O, and S. In some embodiments, n is 1 in Formula (I) and subembodiments thereof n is 1 and R¹ is a 5-membered heterocycloalkyl having 1 to 4 heteroatoms as ring vertices independently selected from N, O, and S. In some embodiments, n is 1 in Formula (I) and subembodiments thereof n is 1 and R¹ is a 4-membered heterocycloalkyl having 1 to 4 heteroatoms as ring vertices independently selected from N, O, and S. In some embodiments, n is 1 in Formula (I) and subembodiments thereof n is 1 and R¹ is selected from the group consisting of azetidinyl, pyrrolidinyl, pyrazolidinyl, piperidinyl, and piperazinyl. In some embodiments, n is 1 in Formula (I) and subembodiments thereof n is 1 and R¹ is azetidinyl.

In some embodiments, n is 1 in Formula (I) and subembodiments thereof and R¹ is selected from the group consisting of C₁₋₆ alkyl, halo, C₁₋₆ haloalkyl, —X¹—O—C₁₋₆ alkyl, C₁₋₆ haloalkoxy, —X¹-cyano, —X¹—OH, C₃₋₆ cycloalkyl, and —X¹—NR^{a}R^{b}. In some embodiments, n is 1 in Formula (I) and subembodiments thereof and R¹ is selected from the group consisting of C₁₋₆ alkyl, halo, —X¹-cyano, and C₁₋₆ haloalkyl. In some embodiments, n is 1 in Formula (I) and subembodiments thereof and R¹ is selected from the group consisting of —X¹—O—C₁₋₆ alkyl, C₁₋₆ haloalkoxy, —X¹—OH, and —X¹—NR^{a}R^{b}. In some embodiments, n is 1 in Formula (I) and subembodiments thereof and R¹ is selected from the group consisting of —X¹—O—C₃₋₆ cycloalkyl, and X¹—X^{1a}—OR¹. In some embodiments, n is 1 in Formula (I) and subembodiments thereof and R¹ is selected from the group consisting of C₁₋₆ alkyl, halo, —X¹-cyano, and C₁₋₆ haloalkyl, —X¹—O—C₃₋₆ cycloalkyl, and X¹—X^{1a}—OR^{a}. In some embodiments, n is 1 in Formula (I) and subembodiments thereof and R¹ is selected from the group consisting of C₁₋₆ alkyl, halo, —X¹-cyano, and C₁₋₆ haloalkyl, and —X¹—O—C₃₋₆ cycloalkyl.

In some embodiments, n is 1 in Formula (I) and subembodiments thereof and R¹ is selected from the group consisting of C₁₋₆ alkyl and —X¹—OH.

In some embodiments, n is 1 in Formula (I) and subembodiments thereof and R¹ is selected from the group consisting of methyl and hydroxymethyl. In some embodiments, n is 1 in Formula (I) and subembodiments thereof and R¹ is selected from the group consisting of ethyl and hydroxyethyl.

In some embodiments, n is 1 in Formula (I) and subembodiments thereof and R¹ is methyl. In some embodiments, n is 1 in Formula (I) and subembodiments thereof and R¹ is ethyl or propyl. In some embodiments, n is 1 in Formula (I) and subembodiments thereof and R¹ is O-cyclopropyl. In some embodiments, n is 1 in Formula (I) and subembodiments thereof and R¹ is O-cyclopropyl. In some embodiments, n is 1 in Formula (I) and subembodiments thereof and R¹ is —CH₂—O-cyclopropyl.

In some embodiments, m is 1 in Formula (I) and R² is C₁₋₆ alkyl, halo, C₁₋₆ haloalkyl, and C₁₋₆ haloalkoxy. In some embodiments, m is 1 in Formula (I) and R² is C₁₋₆ alkyl, halo, and C₁₋₆ haloalkyl.

In some embodiments, m in Formula (I) and subembodiments thereof is 0. In some embodiments, m in Formula (I) and subembodiments thereof is 1.

In some embodiments, each X¹ in Formula (I) and subembodiments thereof is a bond. In some embodiments, each X¹ in Formula (I) and subembodiments thereof is C₁₋₄ alkylene. In some embodiments, each X¹ in Formula (I) and subembodiments thereof is C₁₋₂ alkylene. In some embodiments, each X¹ in Formula (I) and subembodiments thereof is methylene.

In some embodiments, each R^{a} and R^{b} in Formula (I) and subembodiments thereof is hydrogen. In some embodiments, each R^{a} is hydrogen and each R^{b} in Formula (I) and subembodiments thereof is C₁₋₄ alkyl or C₁₋₄ haloalkyl. In some embodiments, each R^{a} and R^{b} in Formula (I) and subembodiments thereof is C₁₋₄ alkyl. In some embodiments, each R^{a} and R^{b} in Formula (I) and subembodiments thereof is C₁₋₂ alkyl. In some embodiments, each R^{a} and R^{b} in Formula (I) and subembodiments thereof is methyl. In some embodiments, each R^{a} and R^{b} in Formula (I) and subembodiments thereof is C₁₋₄ haloalkyl. In some embodiments, each R^{a} and R^{b} in Formula (I) and subembodiments thereof is C₁₋₂ haloalkyl.

In some embodiments, the compound has the Formula (Iaa):

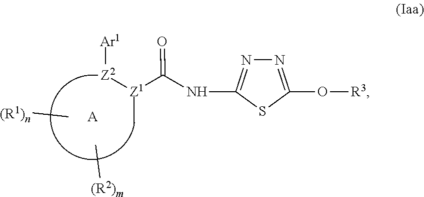

or a pharmaceutically acceptable salt thereof, wherein Z¹ and Z² are ring vertices of ring A linked by a single or a double bond, and each of Z¹ and Z² are carbon atoms.

In some embodiments, the compound has the Formula (Ia):

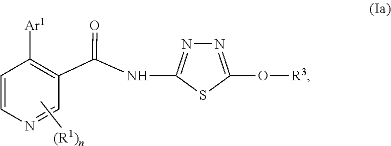

or a pharmaceutically acceptable salt thereof.

In some embodiments, the compound has the Formula (Ib):

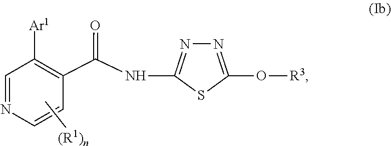

or a pharmaceutically acceptable salt thereof.

In some embodiments, the compound has the Formula (Ic):

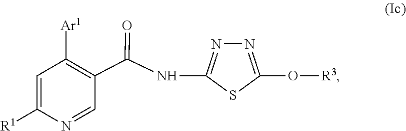

or a pharmaceutically acceptable salt thereof.

In some embodiments, Ar¹ in Formula (I), (Ia), (Ib), and (Ic) is selected from the group consisting of phenyl, pyridinyl, benzopyrazolyl, benzimidazolyl, imidazolyl, pyridazyl, imidazo[1,2-a]pyrimidinyl, oxazolo[4,5-b]pyridinyl, oxazolo[5,4-b]pyridinyl, thiazolo[4,5-b]pyridinyl, benzo[d]thiazole, indazolyl, [1,2,4]triazolo[1,5-a]pyrimidinyl, [1,2,4]triazolo[1,5-b]pyridazinyl, and tetrazolo[1,5-a]pyridinyl, each of which is substituted with 0 to 4 R^{1a}

In some embodiments, Ar¹ in Formula (I), (Ia), (Ib), and (Ic) is selected from the group consisting of phenyl, pyridinyl, benzopyrazolyl, benzimidazolyl, imidazolyl, and pyridazyl each of which is substituted with 0 to 4 R^{1a}.

In some embodiments, Ar¹ in Formula (I), (Ia), (Ib), and (Ic) is selected from the group consisting of 3H-imidazo[4,5-b]pyridinyl, imidazo[1,2-a]pyrimidinyl, oxazolo[4,5-b]pyridinyl, oxazolo[5,4-b]pyridinyl, thiazolo[4,5-b]pyridinyl, benzo[d]thiazole, benzo[c]isothiazolyl, indazolyl, [1,2,4]triazolo[1,5-a]pyrimidinyl, [1,2,4]triazolo[1,5-b]pyridazinyl, tetrazolo[1,5-a]pyridinyl, and [1,2,4]triazolo[1,5-a]pyridinyl, each of which is substituted with 0 to 4 R^{1a}.

In some embodiments, Ar¹ in Formula (I), (Ia), (Ib), and (Ic) is phenyl substituted with 0 to 4 R^{1a}.

In some embodiments, Ar¹ in Formula (I), (Ia), (Ib), and (Ic) is

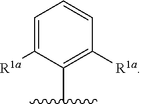

In some embodiments, Ar¹ in Formula (I), (Ia), (Ib), and (Ic) is pyridinyl substituted with 0 to 4 R^{1a}.

In some embodiments, Ar¹ in Formula (I), (Ia), (Ib), and (Ic) is naphthyl substituted with 0 to 4 R^{1a}.

In some embodiments, Ar¹ in Formula (I), (Ia), (Ib), and (Ic) is pyridin-2-one substituted with 0 to 4 R^{1a}.

In some embodiments, Ar¹ in Formula (I), (Ia), (Ib), and (Ic) is naphthyl substituted with 0 to 4 R^{1a}.

In some embodiments, Ar¹ in Formula (I), (Ia), (Ib), and (Ic) is pyridin-2-one substituted with 0 to 4 R^{1a}.

In some embodiments, Ar¹ in Formula (I), (Ia), (Ib), and (Ic) is substituted with 0 to 3 R^{1a}.

In some embodiments, Ar¹ in Formula (I), (Ia), (Ib), and (Ic) is substituted with 0 to 2 R^{1a}.

In some embodiments, the compound has the Formula (Ia1):

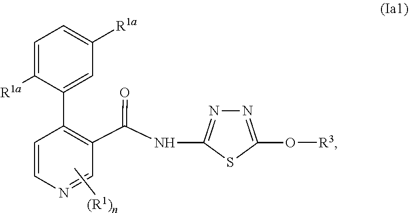

or a pharmaceutically acceptable salt thereof.

In some embodiments, the compound has the Formula (Ib1):

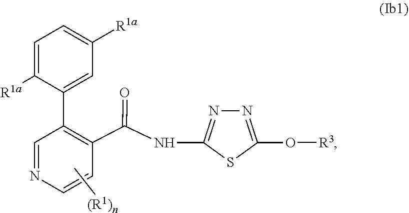

or a pharmaceutically acceptable salt thereof.

In some embodiments, the compound has the Formula (Ic1):

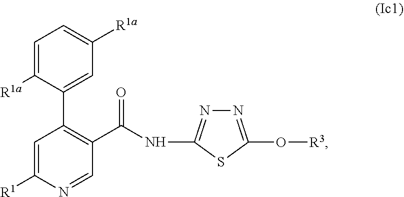

or a pharmaceutically acceptable salt thereof.

In some embodiments, the compound has the Formula (Ia2):

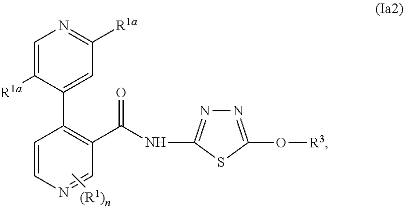

or a pharmaceutically acceptable salt thereof.

In some embodiments, the compound has the Formula (Ib2):

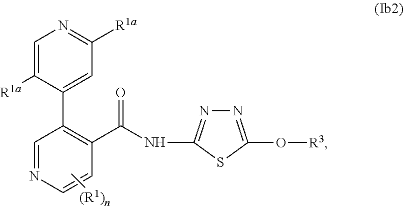

or a pharmaceutically acceptable salt thereof.

In some embodiments, the compound has the Formula (Ic2):

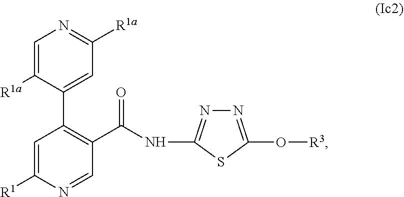

or a pharmaceutically acceptable salt thereof.

In some embodiments, each R^{1a} in Formula (I) and subembodiments thereof is independently selected from C₁₋₆ alkyl, halo, C₁₋₆ haloalkyl, —X²—O—C₁₋₆ alkyl, C₁₋₆ haloalkoxy, C₃₋₆ cycloalkyl, —NR^{a}R^{b}, —X²-cyano, and —X²—OH. In some embodiments, each R^{1a} in Formula (I) and subembodiments thereof is independently selected from C₁₋₆ alkyl, halo, C₁₋₆ haloalkyl, —X²—O—C₁₋₆ alkyl, C₁₋₆ haloalkoxy, C₃₋₆ cycloalkyl, —NR^{a}R^{b}, —X²-cyano, —X²—OH, and —X²—S(O)R^{c}.

In some embodiments, each R^{1a} in Formula (I) and subembodiments thereof is independently selected from C₁₋₆ alkyl, halo, C₁₋₆ haloalkyl, —O—C₁₋₆ alkyl, C₁₋₆ haloalkoxy, C₃₋₆ cycloalkyl, —NR^{a}R^{b}, and —X²—OH. In some embodiments, each R^{1a} in Formula (I) and subembodiments thereof is independently selected from C₁₋₆ alkyl, halo, C₁₋₆ haloalkyl, —X²—O—C₁₋₆ alkyl, C₁₋₆ haloalkoxy, C₃₋₆ cycloalkyl, —NR^{a}R^{b}, —X²-cyano, —X²—OH, and —S(O)R^{c}.

In some embodiments, each R^{1a} in Formula (I) and subembodiments thereof is independently selected from methyl, ethyl, fluoro, chloro, bromo, trifluoromethyl, difluoromethyl, methoxy, ethoxy, difluoromethoxy, cyclopropyl, —NH₂, hydroxymethyl, and 1-hydroxyethyl. In some embodiments, each R^{1a} in Formula (I) and subembodiments thereof is independently selected from methyl, ethyl, fluoro, chloro, bromo, trifluoromethyl, difluoromethyl, methoxy, ethoxy, difluoromethoxy, cyclopropyl, —NH₂, hydroxymethyl, 1-hydroxyethyl and —S(═O)CH₃. In some embodiments, each R^{1a} in Formula (I) and subembodiments thereof is independently selected from methyl, ethyl, fluoro, chloro, bromo, trifluoromethyl, difluoromethyl, methoxy, ethoxy, and difluoromethoxy. In some embodiments, each R^{1a} in Formula (I) and subembodiments thereof is independently selected from ethyl, fluoro, chloro, difluoromethyl, and ethoxy. In some embodiments, each R^{1a} in Formula (I) and subembodiments thereof is independently selected from methyl, fluoro, chloro, trifluoromethyl, difluoromethyl, methoxy, and difluoromethoxy. In some embodiments, each R^{1a} in Formula (I) and subembodiments thereof is independently selected methyl, ethyl, fluoro, cyclopropyl, —NH₂, hydroxymethyl, and 1-hydroxyethyl.

In some embodiments, each R^{1a} in Formula (I) and subembodiments thereof is independently selected from C₁₋₆ alkyl, halo, —O—C₁₋₆ alkyl, —C(O)₂R^{c}, —C₁₋₄ alkoxy-C₁₋₄ alkoxy, —X²—C(O)NR^{c}R^{d}, —X²—S(O)₂NR^{c}R^{d}, —O—X²-cyano, —X²—S(O)₂R^{c}, and —X²—N(R^{d})S(O)₂R^{c}. In some embodiments, each R^{1a} in Formula (I) and subembodiments thereof is independently selected from —C(O)₂R^{c}, —C₁₋₄ alkoxy-C₁₋₄ alkoxy, —X²—C(O)NR^{c}R^{d}, —X²—S(O)₂NR^{c}R^{d}, —O—X²-cyano, —X²—S(O)₂R^{c}, and —X²—N(R^{d})S(O)₂R^{c}.

In some embodiments, each R^{1a} in Formula (I) and subembodiments thereof, wherein at least one R^{1a} is Y and Y is selected from phenyl, benzyl, 4- to 6-membered heterocycloalkyl, and 5- or 6-membered heteroaryl, wherein each heterocycloalkyl and heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl.

In some embodiments, each R^{1a} in Formula (I) and subembodiments thereof, wherein at least one R^{1a} is Y and Y is phenyl or benzyl; and each Y is substituted with 0, 1 or 2 groups independently selected from halo, oxo, C₁₋₄ alkyl, C₁₋₄ alkoxy, and C₁₋₄ haloalkyl.

In some embodiments, each R^{1a} in Formula (I) and subembodiments thereof, wherein at least one R^{1a} is Y and Y is 4- to 6-membered heterocycloalkyl or 5- or 6-membered heteroaryl, wherein each heterocycloalkyl and heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl.

In some embodiments, each R^{1a} in Formula (I) and subembodiments thereof, wherein at least one R^{1a} is Y and Y is 4- to 6-membered heterocycloalkyl, wherein each heterocycloalkyl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl. In some embodiments, the 4- to 6-membered heterocycloalkyl is selected from the group consisting of piperidinyl, morpholinyl, and tetrahydropyranyl.

In some embodiments, each R^{1a} in Formula (I) and subembodiments thereof, wherein at least one R^{1a} is Y and Y is 5- or 6-membered heteroaryl, wherein each heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl. In some embodiments, each R^{1a} in Formula (I) and subembodiments thereof, wherein at least one R^{1a} is Y and Y is 5-membered heteroaryl, wherein each heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl. In some embodiments, the 5- to 6-membered heteroaryl is pyrazolyl.

In some embodiments, two R^{1a} groups, in Formula (I) and subembodiments thereof, on adjacent ring vertices combine to form a 4 to 6 membered heterocycloalkyl having 1 to 2 heteroatoms as ring vertices independently selected from N, O, and S. In some embodiments, two R^{1a} groups, in Formula (I) and subembodiments thereof, on adjacent ring vertices combine to form a 5 membered heterocycloalkyl having 1 to 2 heteroatoms as ring vertices independently selected from N, O, and S. In some embodiments the heterocycloalkyl ring has 1 or 2 double bonds between ring members. In some embodiments the heterocycloalkyl ring is substituted with 0 to 4 groups independently selected from oxo, halo, C₁₋₄ alkyl, C₁₋₄ alkoxy, and C₁₋₄ haloalkyl.

In some embodiments, two R^{1a} groups, in Formula (I) and subembodiments thereof, on adjacent ring vertices combine to form a 4 to 6 membered cycloalkyl. In some embodiments the cycloalkyl ring has 1 or 2 double bonds between ring members. In some embodiments the cycloalkyl ring is substituted with 0 to 4 groups independently selected from oxo, halo, C₁₋₄ alkyl, C₁₋₄ alkoxy, and C₁₋₄ haloalkyl. In some embodiments, each R^{1a} in Formula (I) and subembodiments thereof is independently selected from methyl, fluoro, chloro, trifluoromethyl, difluoromethyl, methoxy, ethoxy, difluoromethoxy, and cyclopropyl.

In some embodiments, each X² in Formula (I) and subembodiments thereof is a bond. In some embodiments, each X² in Formula (I) and subembodiments thereof is C₁₋₄ alkylene. In some embodiments, each X² in Formula (I) and subembodiments thereof is C₁₋₂ alkylene. In some embodiments, each X² in Formula (I) and subembodiments thereof is methylene.

In some embodiments, each R^{c} and R^{d} in Formula (I) and subembodiments thereof is hydrogen, C₁₋₆ alkyl, C₃₋₅ cycloalkyl and C₁₋₆ haloalkyl. In some embodiments, each R^{c} and R^{d} in Formula (I) and subembodiments thereof is hydrogen, C₁₋₆ alkyl, and C₁₋₆ haloalkyl. In some embodiments, each R^{c} and R^{d} in Formula (I) and subembodiments thereof is hydrogen. In some embodiments, each R^{c} is hydrogen and each R^{d} in Formula (I) and subembodiments thereof is C₁₋₄ alkyl or C₁₋₄ haloalkyl. In some embodiments, each R^{c} and R^{d} in Formula (I) and subembodiments thereof is C₁₋₄ alkyl. In some embodiments, each R^{c} and R^{d} in Formula (I) and subembodiments thereof is C₁₋₂ alkyl. In some embodiments, each R^{c} and R^{d} in Formula (I) and subembodiments thereof is methyl. In some embodiments, each R^{c} and R^{d} in Formula (I) and subembodiments thereof is C₁₋₄ haloalkyl. In some embodiments, each R^{c} and R^{d} in Formula (I) and subembodiments thereof is C₁₋₂ haloalkyl.

In some embodiments, R³ in Formula (I) and subembodiments thereof is C₆₋₁₂ spirocyclyl or C₃₋₆ cycloalkyl, each of which is substituted with 0 to 4 R^{3a}. In some embodiments, R³ in Formula (I) and subembodiments thereof is selected from the group consisting of cyclopropyl, cyclobutyl, cyclopentyl, and cyclohexyl, and is substituted with 0 to 4 R^{3a}. In some embodiments, R³ in Formula (I) and subembodiments thereof is selected from the group consisting of spiro[3.3]heptane, spiro[4.4]nonane, spiro[3.4]octane.

In some embodiments, R³ in Formula (I) and subembodiments thereof is C₆₋₁₁ bridged cycloalkyl, each of which is substituted with 0 to 4 R^{3a}. In some embodiments, the C₆₋₁₁ bridged cycloalkyl has the structure

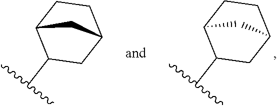 [SMILES: *C1C[C@H]2CC[C@@H]1C2 ; *C1C[C@@H]2CC[C@H]1C2]

each of which is substituted with 0 to 4 R^{3a}.

In some embodiments, R³ is a 3- to 6-membered heterocycloalkyl having 1 to 4 heteroatoms as ring vertices independently selected from N, O, and S, and is substituted with 0 to 4 R^{3a}. In some embodiments, R³ in Formula (I) and subembodiments thereof is a 5-membered heterocycloalkyl substituted with 0 to 4 R^{3a}. In some embodiments, R³ in Formula (I) and subembodiments thereof is a 6-membered heterocycloalkyl substituted with 0 to 4 R^{3a}. In some embodiments, R³ in Formula (I) and subembodiments thereof is selected from the group consisting of piperidinyl, piperazinyl, morpholinyl, 2-oxopiperazinyl, 2-tetrahydropyranyl, 3,6-dihydro-2H-pyranyl, 2-oxo-1,2-dihydropyridinyl, thiomorpholinyl, and 1,1-dioxothiomorpholinyl each of which is substituted with 0 to 4 R^{3a}. In some embodiments, R³ in Formula (I) and subembodiments thereof is selected from the group consisting of tetrahydropyran, oxetanyl, tetrahydrofuranyl, and tetrahydrothiopyranyl, each of which is substituted with 0 to 4 R^{3a}. In some embodiments, R³ in Formula (I) and subembodiments thereof is tetrahydrothiopyranyl substituted with 2 oxo groups.

In some embodiments, R³ in Formula (I) and subembodiments thereof is a 6- to 10-membered bicyclic heterocyclyl having 1 to 4 heteroatoms as ring vertices independently selected from N, O, and S, and is substituted with 0 to 4 R^{3a}. In some embodiments, R³ in Formula (I) and subembodiments thereof is selected from the group consisting of 6-oxohexahydropyrrolo[1,2-a]pyrazin-2(1H)-yl and 2,3-dihydro-4H-benzo[b][1,4]oxazin-4-yl and is substituted with 0 to 4 R^{3a}. In some embodiments, R³ in Formula (I) and subembodiments thereof is selected from the group consisting of 6-oxohexahydropyrrolo[1,2-a]pyrazin-2(1H)-yl, 3-oxo-3,4-dihydro-2H-benzo[b][1,4]oxazin-8-yl, benzo[d][1,3]dioxol-4-yl, (3,4-dihydro-2H-1,4-benzoxazin-8-yl), [5H,6H, 7H-pyrazolo[1,5-a]pyrimidin-4-yl] and 2,3-dihydro-4H-benzo[b][1,4]oxazin-4-yl, and is substituted with 0 to 4 R^{3a}.

In some embodiments, R³ in Formula (I) and subembodiments thereof is a 6- to 10-membered bridged heterocyclyl having 1 to 4 heteroatoms as ring vertices independently selected from N, O, and S, and is substituted with 0 to 4 R^{3a}. In some embodiments, R³ in Formula (I) and subembodiments thereof is selected from the group consisting of 2-azabicyclo[2.2.2]octane, quinuclidine, and 7-oxabicyclo[2.2.1]heptane, and is substituted with 0 to 4 R^{3a}.

In some embodiments, R³ in Formula (I) and subembodiments thereof is a 6- to 12-membered spiroheterocyclyl having 1 to 4 heteroatoms as ring vertices independently selected from N, O, and S, and is substituted with 0 to 4 R^{3a}. In some embodiments, R³ in Formula (I) and subembodiments thereof is selected from the group consisting of 4-oxaspiro[2.4]heptane, 2,6-diazaspiro[3.3]heptane, 2,6-diazaspiro[3.4]octane, 2-azaspiro[3.4]octane, 2-azaspiro[3.5]-nonane, and 2,7-diazaspiro[4.4]nonane, and is substituted with 0 to 4 R^{3a}.

In some embodiments, R³ in Formula (I) and subembodiments thereof is substituted with 0 to 2 R^{3a}. In some embodiments. R³ in Formula (I) and subembodiments thereof is substituted with 1 R^{3a}. In some embodiments. R³ in Formula (I) and subembodiments thereof is substituted with 2 R^{3a}. In some embodiments, each R^{3a} in Formula (I) and subembodiments thereof is selected from the group consisting of C₁₋₆ alkyl, halo, —X³—O—C₁₋₆ alkyl, C₁₋₆ haloalkyl, C₁₋₆ haloalkoxy, —X³—OH, —ONO₂, 4- to 6-member heterocycloalkyl having 1 to 4 heteroatoms as ring vertices independently selected from N, O, and S and oxo. In some embodiments, each R^{3a} in Formula (I) and subembodiments thereof is selected from the group consisting of C₁₋₆ alkyl, halo, —X³—O—C₁₋₆ alkyl, C₁₋₆ haloalkyl, C₁₋₆ haloalkoxy, —X³—OH, and oxo. In some embodiments, each R^{3a} in Formula (I) and subembodiments thereof is selected from the group consisting of C₁₋₆ alkyl, halo, —X³—OH, and oxo. In some embodiments, each R^{3a} in Formula (I) and subembodiments thereof is selected from the group consisting of methyl, —OH, and oxo. In some embodiments, at least one R^{3a} in Formula (I) and subembodiments thereof is selected from the group consisting of a 4- to 6-member heterocycloalkyl having 1 to 4 heteroatoms as ring vertices independently selected from N, O, and S and oxo. In some embodiments, at least one R^{3a} in Formula (I) and subembodiments thereof is selected from the group consisting of tetrahydropyranyl and oxetanyl.

In some embodiments. R³ is in Formula (I) and subembodiments thereof hydrogen.

In some embodiments, R³ is in Formula (I) and subembodiments thereof C₁₋₆ alkyl or C₂₋₆ alkynyl, and is substituted with 0 to 4 R^{3b}. In some embodiments. R³ in Formula (I) and subembodiments thereof is selected from the group consisting of 2-propynyl, ethyl, methyl, 2,2,-dimethylpropyl, isobutyl, isopropyl, and n-propyl, and is substituted with 0 to 4 R^{3b}.

In some embodiments, R³ in Formula (I) and subembodiments thereof is methyl and is substituted with 0 to 3 R^{3b}. In some embodiments, R³ in Formula (I) and subembodiments thereof is methyl.

In some embodiments, R³ in Formula (I) and subembodiments thereof is C₁₋₆ alkyl or C₂₋₆ alkynyl and is substituted with 0 to 3 R^{3b}. In some embodiments, R³ in Formula (I) and subembodiments thereof is C₁₋₆ alkyl or C₂₋₆ alkynyl and is substituted with 0 to 2 R^{3b}. In some embodiments, R³ in Formula (I) and subembodiments thereof is C₁₋₆ alkyl or C₂₋₆ alkynyl and is substituted with 1 R^{3b}.

In some embodiments, each R^{3b} in Formula (I) and subembodiments thereof is selected from the group consisting of halo, C₁₋₃ haloalkyl, —O—C₁₋₆ alkyl, cyano, —OH, and —CONR^{e}R^{f}. In some embodiments, each R^{3b} in Formula (I) and subembodiments thereof is selected from the group consisting of halo, —O—C₁₋₆ alkyl, cyano, and —OH. In some embodiments, each R^{3b} in Formula (I) and subembodiments thereof is selected from the group consisting of fluoro, methoxy, cyano, and —OH. In some embodiments, each R^{3b} in Formula (I) and subembodiments thereof is selected from the group consisting of —O—C₁₋₆ alkyl, cyano, and —OH. In some embodiments, each R^{3b} in Formula (I) and subembodiments thereof is selected from the group consisting of methoxy, cyano, and —OH.

In some embodiments, each X³ in Formula (I) and subembodiments thereof is a bond. In some embodiments, each X³ in Formula (I) and subembodiments thereof is C₁₋₄ alkylene. In some embodiments, each X³ in Formula (I) and subembodiments thereof is C₁₋₂ alkylene. In some embodiments, each X³ in Formula (I) and subembodiments thereof is methylene.

In some embodiments, each R^{e} and R^{f} in Formula (I) and subembodiments thereof is hydrogen. In some embodiments, each R^{e} is hydrogen and each R^{f} in Formula (I) and subembodiments thereof is C₁₋₄ alkyl or C₁₋₄ haloalkyl. In some embodiments, each R^{e} and R^{f} in Formula (I) and subembodiments thereof is C₁₋₄ alkyl. In some embodiments, each R^{e} and R^{f} in Formula (I) and subembodiments thereof is C₁₋₂ alkyl. In some embodiments, each R^{e} and R^{f} in Formula (I) and subembodiments thereof is methyl. In some embodiments, each R^{e} and R^{f} in Formula (I) and subembodiments thereof is C₁₋₄ haloalkyl. In some embodiments, each R^{e} and R^{f} in Formula (I) and subembodiments thereof is C₁₋₂ haloalkyl.

Representative compounds of Formula (I) are listed in Table 1 below:

TABLE 1

| Cpd | Structure |
|---|---|
| # | |
| 1 | 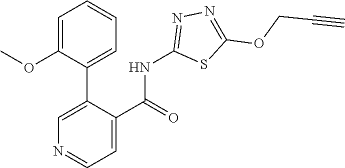 [SMILES: C#CCOc1nnc(NC(=O)c2ccncc2-c2ccccc2OC)s1] |
| 2 | 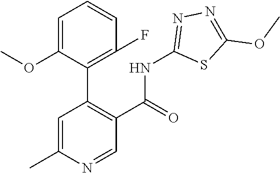 |
| 3 | 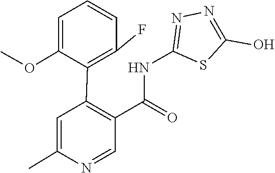 [SMILES: COc1cccc(F)c1-c1cc(C)ncc1C(=O)Nc1nnc(O)s1] |
| 4 | 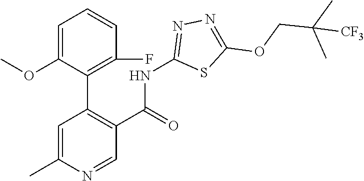 [SMILES: COc1cccc(F)c1-c1cc(C)ncc1C(=O)Nc1nnc(OCC(C)(C)C(F)(F)F)s1] |
| 5 | 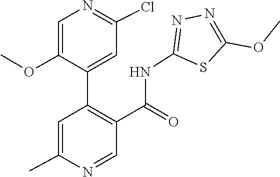 |
| 6 | 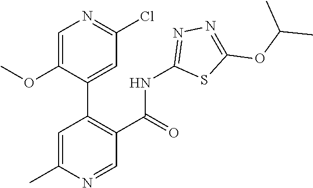 [SMILES: COc1cnc(Cl)cc1-c1cc(C)ncc1C(=O)Nc1nnc(OC(C)C)s1] |
| 7 | 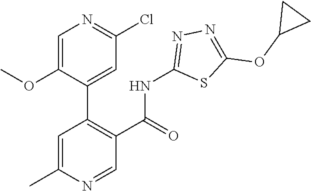 [SMILES: COc1cnc(Cl)cc1-c1cc(C)ncc1C(=O)Nc1nnc(OC2CC2)s1] |
| 8 | 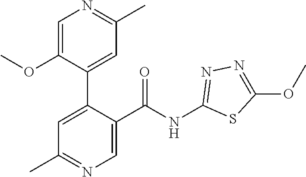 |
| 9 | 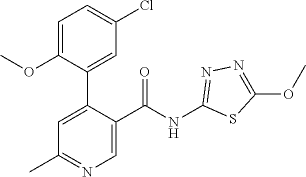 |
| 10 | 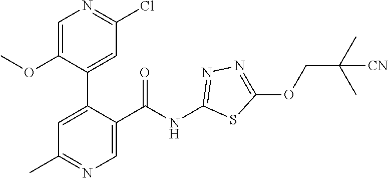 [SMILES: COc1cnc(Cl)cc1-c1cc(C)ncc1C(=O)Nc1nnc(OCC(C)(C)C#N)s1] |
| 11 | 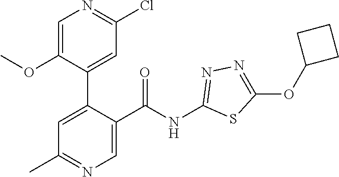 [SMILES: COc1cnc(Cl)cc1-c1cc(C)ncc1C(=O)Nc1nnc(OC2CCC2)s1] |
| 12 | 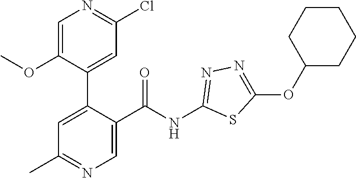 [SMILES: COc1cnc(Cl)cc1-c1cc(C)ncc1C(=O)Nc1nnc(OC2CCCCC2)s1] |
| 13 | 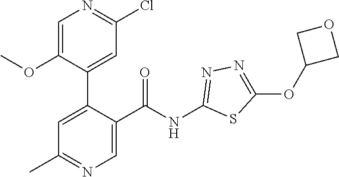 [SMILES: COc1cnc(Cl)cc1-c1cc(C)ncc1C(=O)Nc1nnc(OC2COC2)s1] |
| 14 | 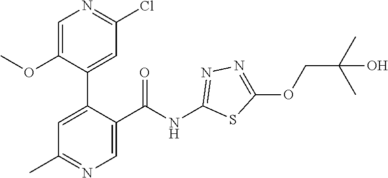 [SMILES: COc1cnc(Cl)cc1-c1cc(C)ncc1C(=O)Nc1nnc(OCC(C)(C)O)s1] |
| 15 | 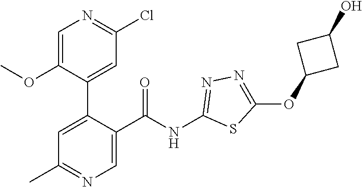 [SMILES: COc1cnc(Cl)cc1-c1cc(C)ncc1C(=O)Nc1nnc(O[C@H]2C[C@@H](O)C2)s1] |
| 16 | 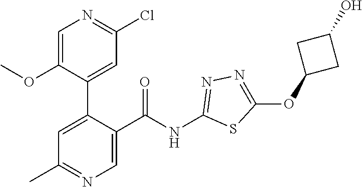 [SMILES: COc1cnc(Cl)cc1-c1cc(C)ncc1C(=O)Nc1nnc(O[C@H]2C[C@H](O)C2)s1] |
| 17 | 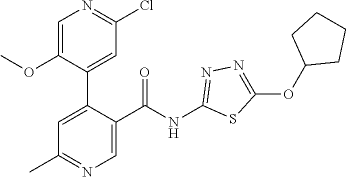 [SMILES: COc1cnc(Cl)cc1-c1cc(C)ncc1C(=O)Nc1nnc(OC2CCCC2)s1] |
| 18 | 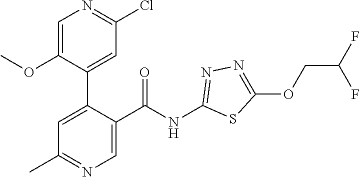 [SMILES: COc1cnc(Cl)cc1-c1cc(C)ncc1C(=O)Nc1nnc(OCC(F)F)s1] |
| 19 | 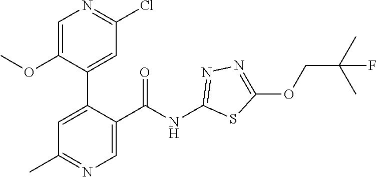 [SMILES: COc1cnc(Cl)cc1-c1cc(C)ncc1C(=O)Nc1nnc(OCC(C)(C)F)s1] |
| 20 | 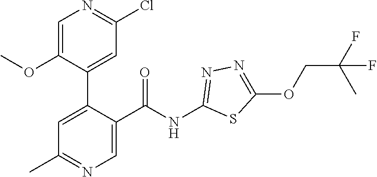 [SMILES: COc1cnc(Cl)cc1-c1cc(C)ncc1C(=O)Nc1nnc(OCC(C)(F)F)s1] |
| 21 | 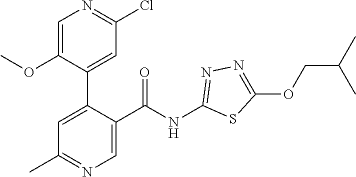 [SMILES: COc1cnc(Cl)cc1-c1cc(C)ncc1C(=O)Nc1nnc(OCC(C)C)s1] |
| 22 | 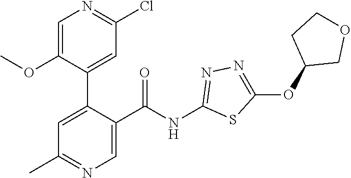 [SMILES: COc1cnc(Cl)cc1-c1cc(C)ncc1C(=O)Nc1nnc(O[C@H]2CCOC2)s1] |
| 23 | 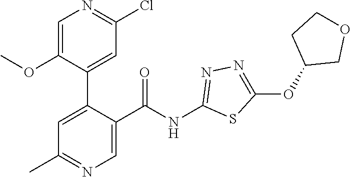 [SMILES: COc1cnc(Cl)cc1-c1cc(C)ncc1C(=O)Nc1nnc(O[C@@H]2CCOC2)s1] |
| 24 | 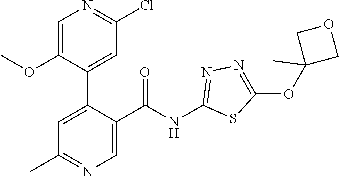 [SMILES: COc1cnc(Cl)cc1-c1cc(C)ncc1C(=O)Nc1nnc(OC2(C)COC2)s1] |
| 25 | 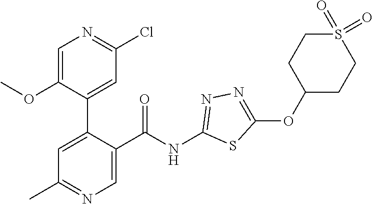 [SMILES: COc1cnc(Cl)cc1-c1cc(C)ncc1C(=O)Nc1nnc(OC2CCS(=O)(=O)CC2)s1] |
| 26 | 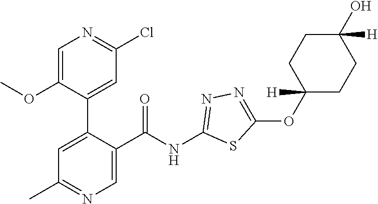 [SMILES: COc1cnc(Cl)cc1-c1cc(C)ncc1C(=O)Nc1nnc(O[C@H]2CC[C@@H](O)CC2)s1] |
| 27 | 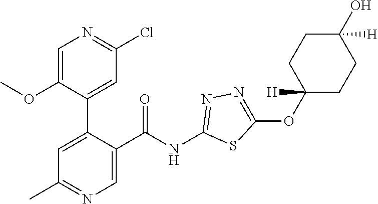 [SMILES: COc1cnc(Cl)cc1-c1cc(C)ncc1C(=O)Nc1nnc(O[C@H]2CC[C@H](O)CC2)s1] |
| 28 | 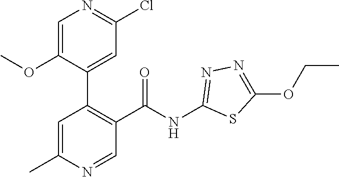 [SMILES: CCOc1nnc(NC(=O)c2cnc(C)cc2-c2cc(Cl)ncc2OC)s1] |
| 29 | 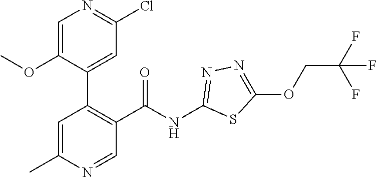 [SMILES: COc1cnc(Cl)cc1-c1cc(C)ncc1C(=O)Nc1nnc(OCC(F)(F)F)s1] |
| 30 | 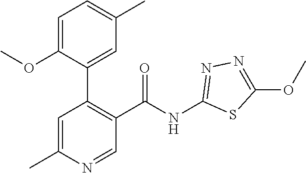 |
| 31 | 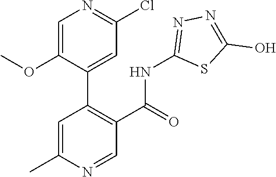 [SMILES: COc1cnc(Cl)cc1-c1cc(C)ncc1C(=O)Nc1nnc(O)s1] |
| 32 | 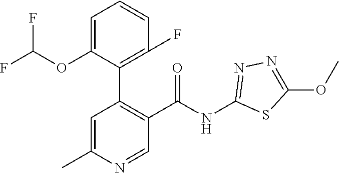 [SMILES: COc1nnc(NC(=O)c2cnc(C)cc2-c2c(F)cccc2OC(F)F)s1] |
| 33 | 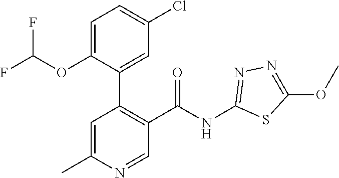 [SMILES: COc1nnc(NC(=O)c2cnc(C)cc2-c2cc(Cl)ccc2OC(F)F)s1] |
| 34 | 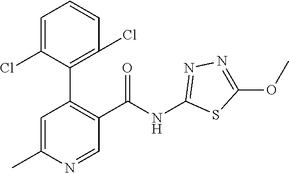 [SMILES: COc1nnc(NC(=O)c2cnc(C)cc2-c2c(Cl)cccc2Cl)s1] |
| 35 | 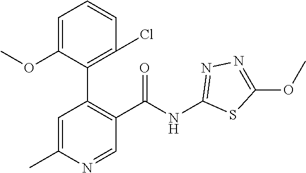 |
| 36 | 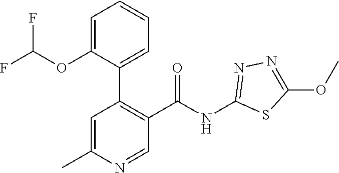 [SMILES: COc1nnc(NC(=O)c2cnc(C)cc2-c2ccccc2OC(F)F)s1] |
| 37 | 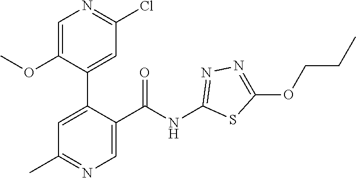 [SMILES: CCCOc1nnc(NC(=O)c2cnc(C)cc2-c2cc(Cl)ncc2OC)s1] |
| 38 | 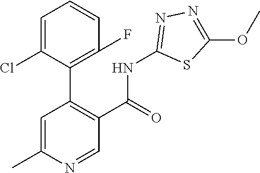 [SMILES: COc1nnc(NC(=O)c2cnc(C)cc2-c2c(F)cccc2Cl)s1] |
| 39 | 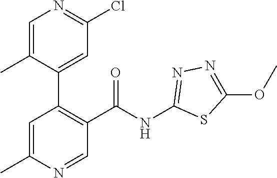 |
| 40 | 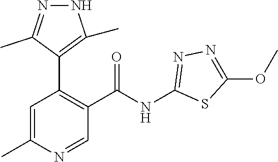 |
| 41 | 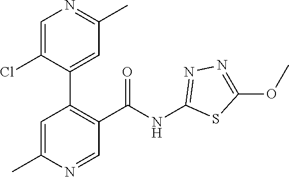 [SMILES: COc1nnc(NC(=O)c2cnc(C)cc2-c2cc(C)ncc2Cl)s1] |
| 42 | 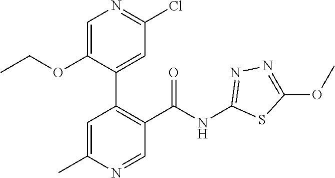 [SMILES: CCOc1cnc(Cl)cc1-c1cc(C)ncc1C(=O)Nc1nnc(OC)s1] |
| 43 | 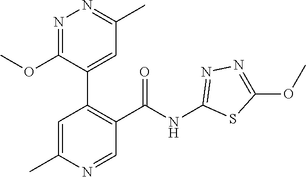 |
| 44 | 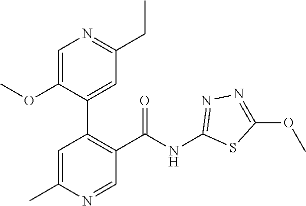 |
| 45 | 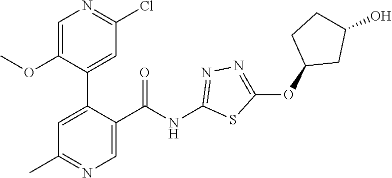 [SMILES: COc1cnc(Cl)cc1-c1cc(C)ncc1C(=O)Nc1nnc(O[C@H]2CC[C@H](O)C2)s1] |
| 46 | 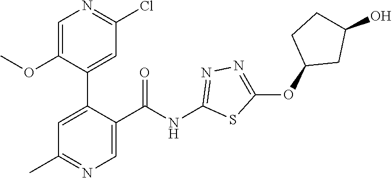 [SMILES: COc1cnc(Cl)cc1-c1cc(C)ncc1C(=O)Nc1nnc(O[C@H]2CC[C@@H](O)C2)s1] |
| 47 | 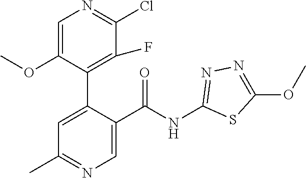 |
| 48 | 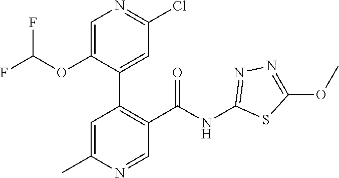 [SMILES: COc1nnc(NC(=O)c2cnc(C)cc2-c2cc(Cl)ncc2OC(F)F)s1] |
| 49 | 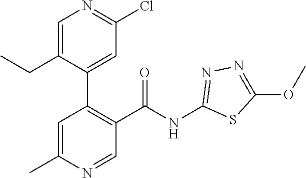 [SMILES: CCc1cnc(Cl)cc1-c1cc(C)ncc1C(=O)Nc1nnc(OC)s1] |
| 50 | 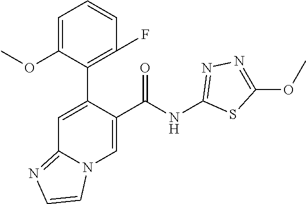 |
| 51 | 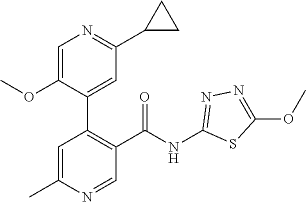 |
| 52 | 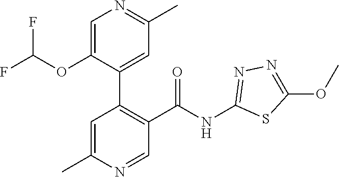 [SMILES: COc1nnc(NC(=O)c2cnc(C)cc2-c2cc(C)ncc2OC(F)F)s1] |
| 53 | 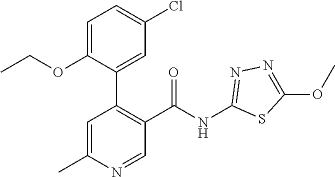 [SMILES: CCOc1ccc(Cl)cc1-c1cc(C)ncc1C(=O)Nc1nnc(OC)s1] |
| 54 | 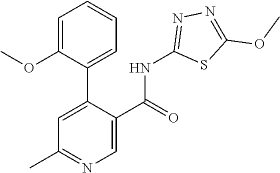 |
| 55 | 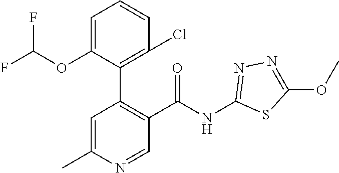 [SMILES: COc1nnc(NC(=O)c2cnc(C)cc2-c2c(Cl)cccc2OC(F)F)s1] |
| 56 | 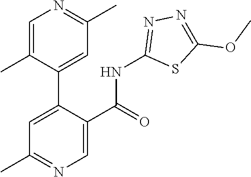 |
| 57 | 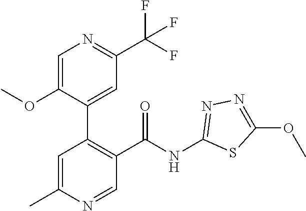 |
| 58 | 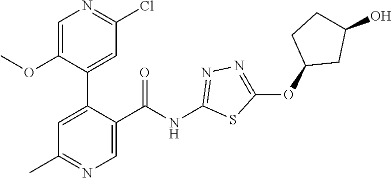 [SMILES: COc1cnc(Cl)cc1-c1cc(C)ncc1C(=O)Nc1nnc(O[C@H]2CC[C@@H](O)C2)s1] |
| 59 | 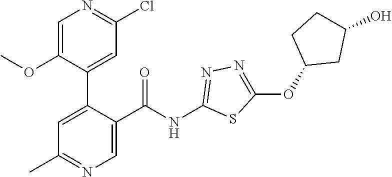 [SMILES: COc1cnc(Cl)cc1-c1cc(C)ncc1C(=O)Nc1nnc(O[C@@H]2CC[C@H](O)C2)s1] |
| 60 | 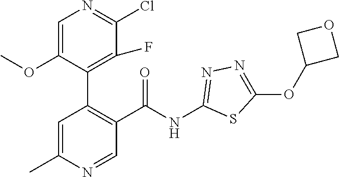 [SMILES: COc1cnc(Cl)c(F)c1-c1cc(C)ncc1C(=O)Nc1nnc(OC2COC2)s1] |
| 61 | 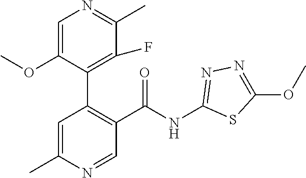 |
| 62 | 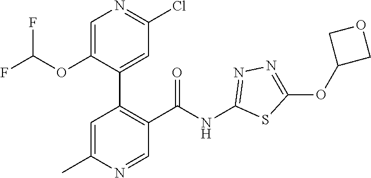 [SMILES: Cc1cc(-c2cc(Cl)ncc2OC(F)F)c(C(=O)Nc2nnc(OC3COC3)s2)cn1] |
| 63 | 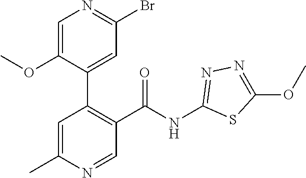 |
| 64 | 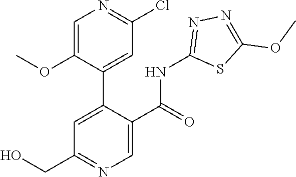 |
| 65 | 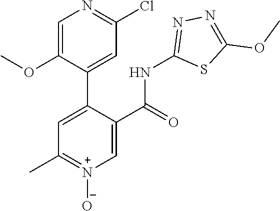 |
| 66 | 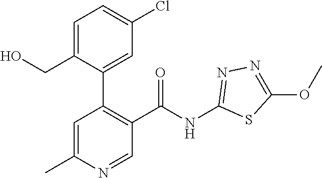 [SMILES: COc1nnc(NC(=O)c2cnc(C)cc2-c2cc(Cl)ccc2CO)s1] |
| 67 | 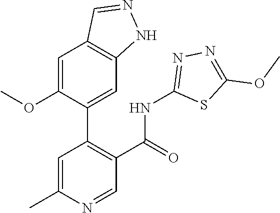 |
| 68 | 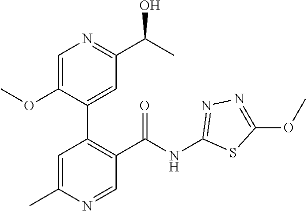 |
| 69 | 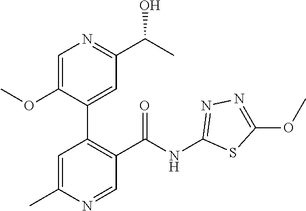 |
| 70 | 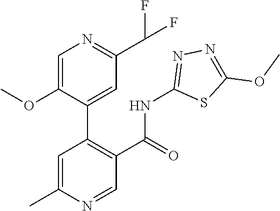 |
| 71 | 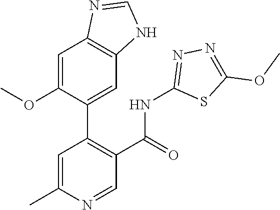 |
| 72 | 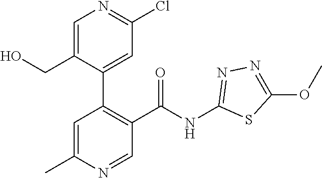 [SMILES: COc1nnc(NC(=O)c2cnc(C)cc2-c2cc(Cl)ncc2CO)s1] |
| 73 | 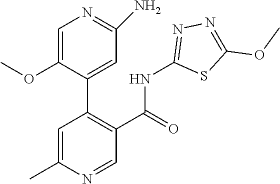 |
| 74 | 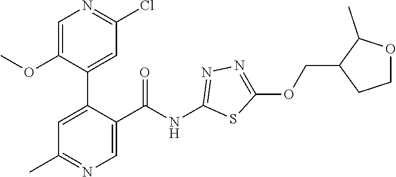 [SMILES: COc1cnc(Cl)cc1-c1cc(C)ncc1C(=O)Nc1nnc(OCC2CCOC2C)s1] |
| 75 | 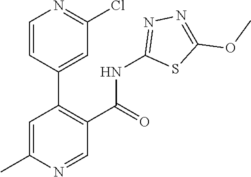 |
| 76 | 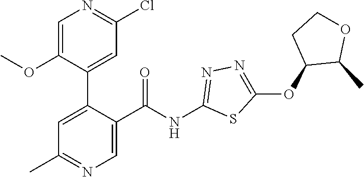 |
| 77 | 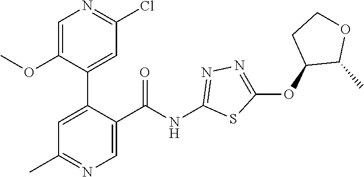 |
| 78 | 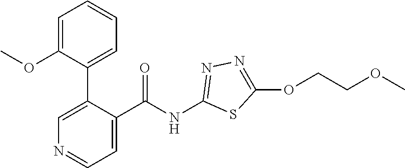 |

Additional representative compounds of Formula (I) are listed in Table 2 below:

TABLE 2

| Cpd | |
|---|---|
| # | Structure |
| 79 | 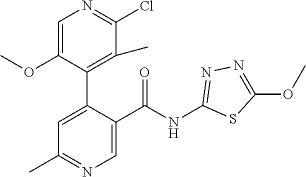 |
| 80 | 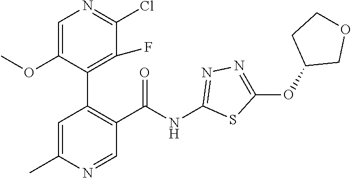 [SMILES: COc1cnc(Cl)c(F)c1-c1cc(C)ncc1C(=O)Nc1nnc(O[C@@H]2CCOC2)s1] |
| 81 | 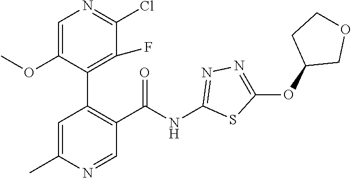 [SMILES: COc1cnc(Cl)c(F)c1-c1cc(C)ncc1C(=O)Nc1nnc(O[C@H]2CCOC2)s1] |
| 82 | 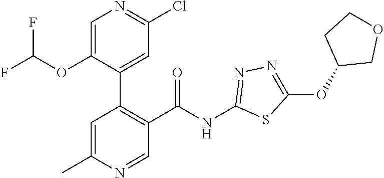 [SMILES: Cc1cc(-c2cc(Cl)ncc2OC(F)F)c(C(=O)Nc2nnc(O[C@@H]3CCOC3)s2)cn1] |
| 83 | 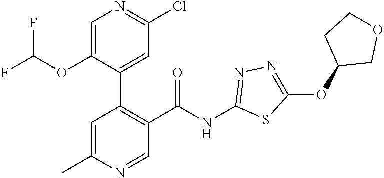 [SMILES: Cc1cc(-c2cc(Cl)ncc2OC(F)F)c(C(=O)Nc2nnc(O[C@H]3CCOC3)s2)cn1] |
| 84 | 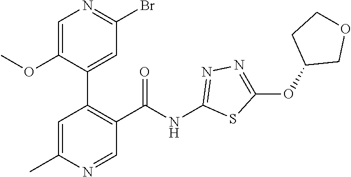 [SMILES: COc1cnc(Br)cc1-c1cc(C)ncc1C(=O)Nc1nnc(O[C@@H]2CCOC2)s1] |
| 85 | 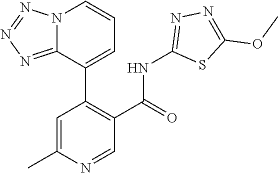 [SMILES: COc1nnc(NC(=O)c2cnc(C)cc2-c2cccn3nnnc23)s1] |
| 86 | 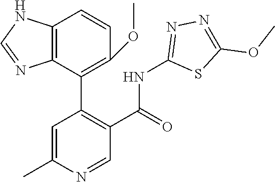 [SMILES: COc1nnc(NC(=O)c2cnc(C)cc2-c2c(OC)ccc3[nH]cnc23)s1] |
| 87 | 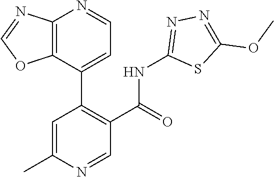 [SMILES: COc1nnc(NC(=O)c2cnc(C)cc2-c2ccnc3ncoc23)s1] |
| 88 | 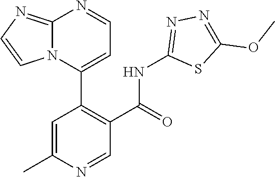 [SMILES: COc1nnc(NC(=O)c2cnc(C)cc2-c2ccnc3nccn23)s1] |
| 89 | 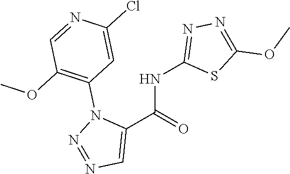 |
| 90 | 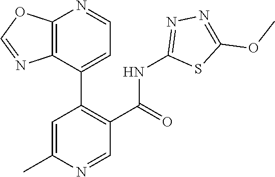 [SMILES: COc1nnc(NC(=O)c2cnc(C)cc2-c2ccnc3ocnc23)s1] |
| 91 | 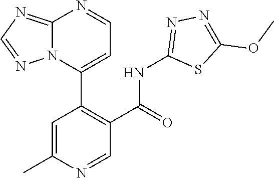 [SMILES: COc1nnc(NC(=O)c2cnc(C)cc2-c2ccnc3ncnn23)s1] |
| 92 | 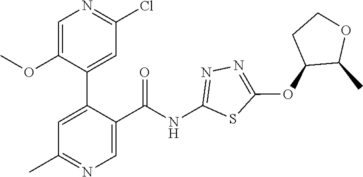 |
| 93 | 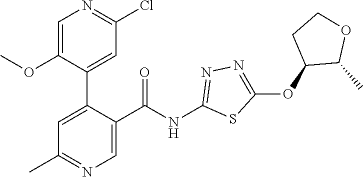 |
| 94 | 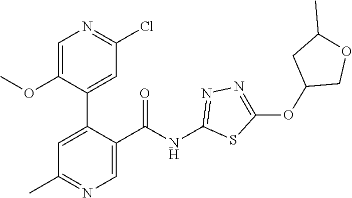 [SMILES: COc1cnc(Cl)cc1-c1cc(C)ncc1C(=O)Nc1nnc(OC2COC(C)C2)s1] |
| 95 | 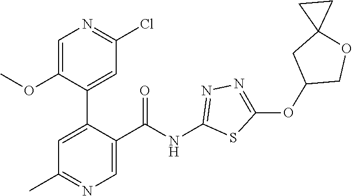 [SMILES: COc1cnc(Cl)cc1-c1cc(C)ncc1C(=O)Nc1nnc(OC2COC3(CC3)C2)s1] |
| 96 | 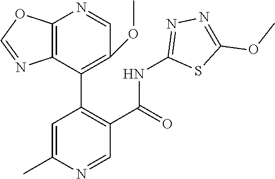 [SMILES: COc1nnc(NC(=O)c2cnc(C)cc2-c2c(OC)cnc3ocnc23)s1] |
| 97 | 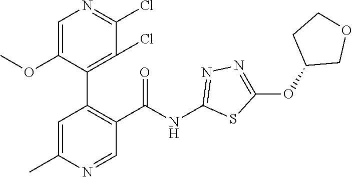 [SMILES: COc1cnc(Cl)c(Cl)c1-c1cc(C)ncc1C(=O)Nc1nnc(O[C@@H]2CCOC2)s1] |
| 98 | 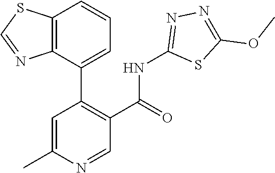 [SMILES: COc1nnc(NC(=O)c2cnc(C)cc2-c2cccc3scnc23)s1] |
| 99 | 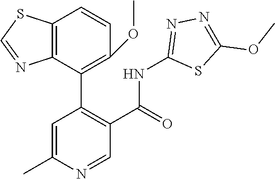 [SMILES: COc1nnc(NC(=O)c2cnc(C)cc2-c2c(OC)ccc3scnc23)s1] |
| 100 | 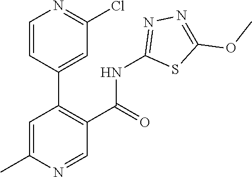 |
| 101 | 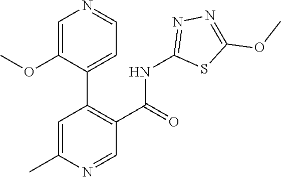 |
| 102 | 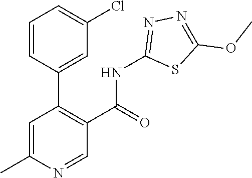 |
| 103 | 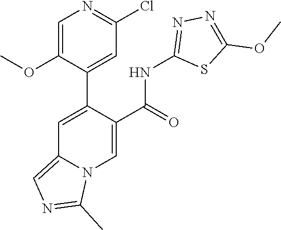 |
| 104 | 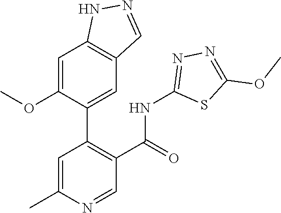 |
| 105 | 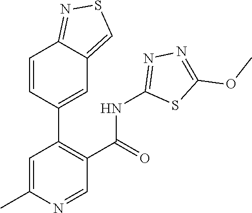 |
| 106 | 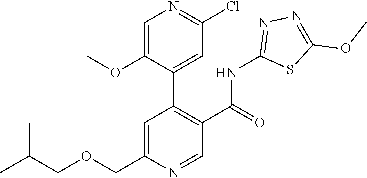 [SMILES: COc1nnc(NC(=O)c2cnc(COCC(C)C)cc2-c2cc(Cl)ncc2OC)s1] |
| 107 | 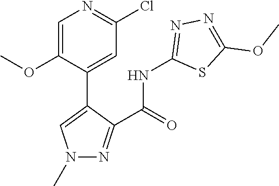 |
| 108 | 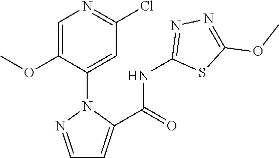 |
| 109 | 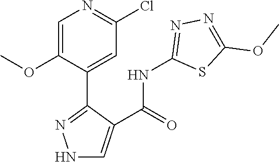 |
| 110 | 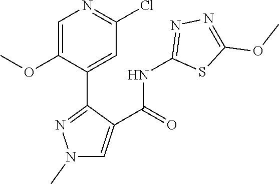 |
| 111 | 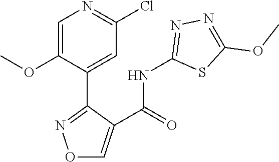 |
| 112 | 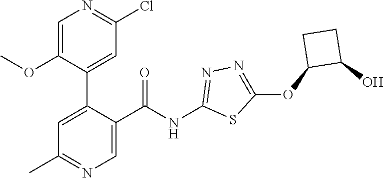 [SMILES: COc1cnc(Cl)cc1-c1cc(C)ncc1C(=O)Nc1nnc(O[C@H]2CC[C@H]2O)s1] |
| 113 | 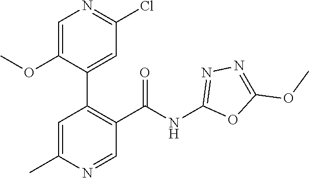 |
| 114 | 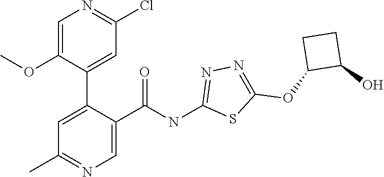 |
| 115 | 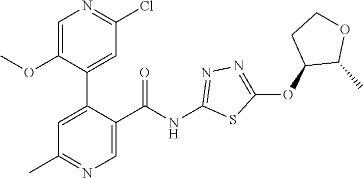 |
| 116 | 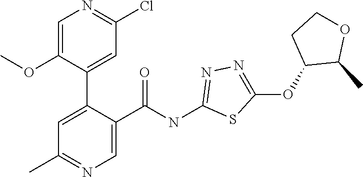 |
| 117 | 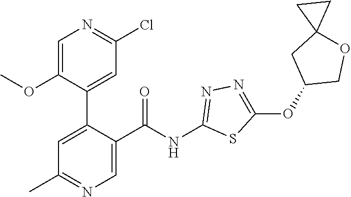 [SMILES: COc1cnc(Cl)cc1-c1cc(C)ncc1C(=O)Nc1nnc(O[C@H]2COC3(CC3)C2)s1] |
| 118 | 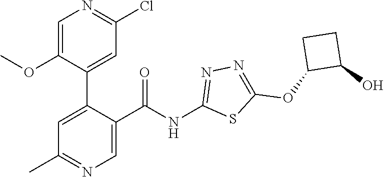 [SMILES: COc1cnc(Cl)cc1-c1cc(C)ncc1C(=O)Nc1nnc(O[C@@H]2CC[C@H]2O)s1] |
| 119 | 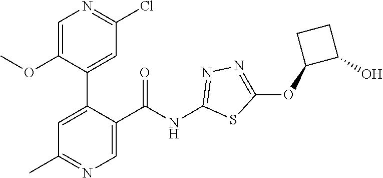 [SMILES: COc1cnc(Cl)cc1-c1cc(C)ncc1C(=O)Nc1nnc(O[C@H]2CC[C@@H]2O)s1] |
| 120 | 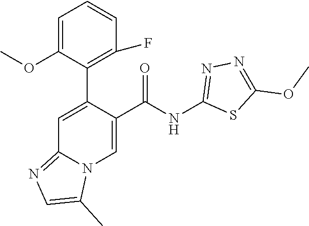 |
| 121 | 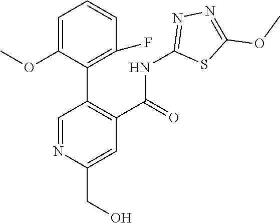 |
| 122 | 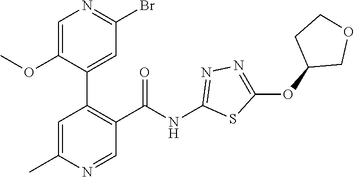 [SMILES: COc1cnc(Br)cc1-c1cc(C)ncc1C(=O)Nc1nnc(O[C@H]2CCOC2)s1] |
| 123 | 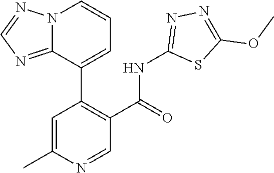 [SMILES: COc1nnc(NC(=O)c2cnc(C)cc2-c2cccn3ncnc23)s1] |
| 124 | 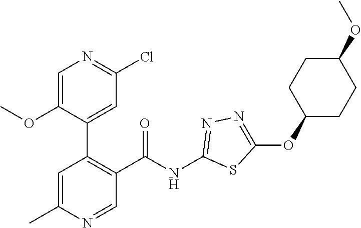 [SMILES: COc1cnc(Cl)cc1-c1cc(C)ncc1C(=O)Nc1nnc(O[C@H]2CC[C@@H](OC)CC2)s1] |
| 125 | 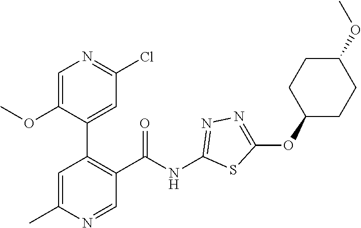 [SMILES: COc1cnc(Cl)cc1-c1cc(C)ncc1C(=O)Nc1nnc(O[C@H]2CC[C@H](OC)CC2)s1] |
| 126 | 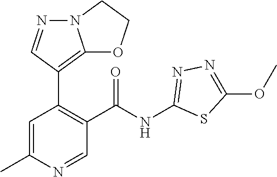 |
| 127 | 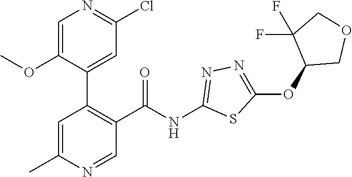 [SMILES: COc1cnc(Cl)cc1-c1cc(C)ncc1C(=O)Nc1nnc(O[C@@H]2COCC2(F)F)s1] |
| 128 | 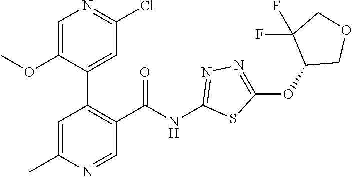 [SMILES: COc1cnc(Cl)cc1-c1cc(C)ncc1C(=O)Nc1nnc(O[C@H]2COCC2(F)F)s1] |
| 129 | 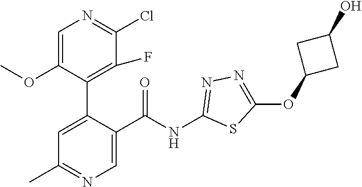 [SMILES: COc1cnc(Cl)c(F)c1-c1cc(C)ncc1C(=O)Nc1nnc(O[C@H]2C[C@@H](O)C2)s1] |
| 130 | 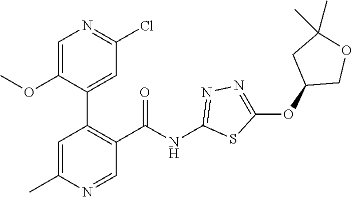 [SMILES: COc1cnc(Cl)cc1-c1cc(C)ncc1C(=O)Nc1nnc(O[C@@H]2COC(C)(C)C2)s1] |
| 131 | 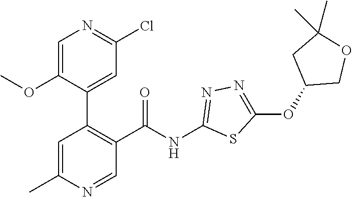 [SMILES: COc1cnc(Cl)cc1-c1cc(C)ncc1C(=O)Nc1nnc(O[C@H]2COC(C)(C)C2)s1] |
| 132 | 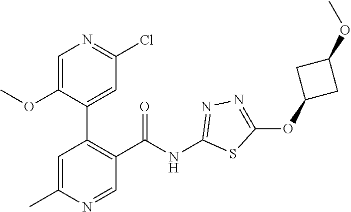 [SMILES: COc1cnc(Cl)cc1-c1cc(C)ncc1C(=O)Nc1nnc(O[C@H]2C[C@@H](OC)C2)s1] |
| 133 | 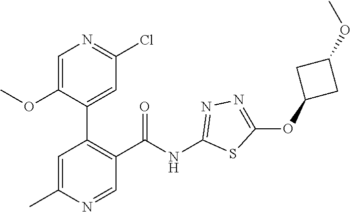 [SMILES: COc1cnc(Cl)cc1-c1cc(C)ncc1C(=O)Nc1nnc(O[C@H]2C[C@H](OC)C2)s1] |
| 134 | 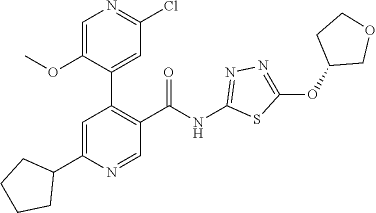 [SMILES: COc1cnc(Cl)cc1-c1cc(C2CCCC2)ncc1C(=O)Nc1nnc(O[C@@H]2CCOC2)s1] |
| 135 | 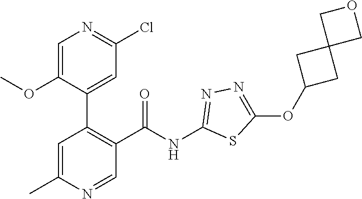 [SMILES: COc1cnc(Cl)cc1-c1cc(C)ncc1C(=O)Nc1nnc(OC2CC3(COC3)C2)s1] |
| 136 | 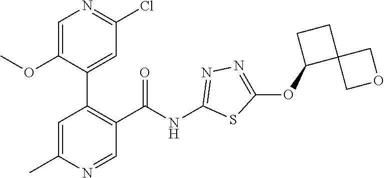 [SMILES: COc1cnc(Cl)cc1-c1cc(C)ncc1C(=O)Nc1nnc(O[C@H]2CCC23COC3)s1] |
| 137 | 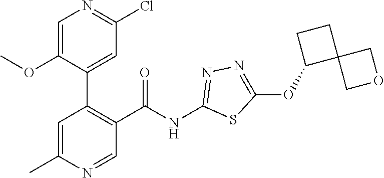 [SMILES: COc1cnc(Cl)cc1-c1cc(C)ncc1C(=O)Nc1nnc(O[C@@H]2CCC23COC3)s1] |
| 138 | 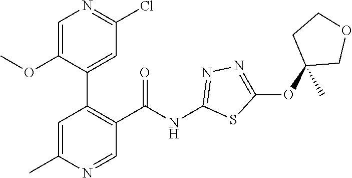 [SMILES: COc1cnc(Cl)cc1-c1cc(C)ncc1C(=O)Nc1nnc(O[C@@]2(C)CCOC2)s1] |
| 139 | 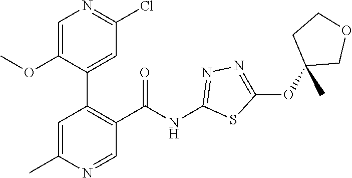 [SMILES: COc1cnc(Cl)cc1-c1cc(C)ncc1C(=O)Nc1nnc(O[C@]2(C)CCOC2)s1] |
| 140 | 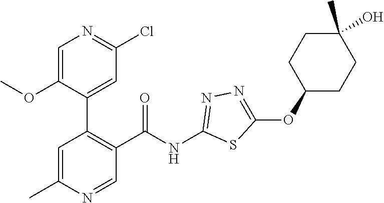 [SMILES: COc1cnc(Cl)cc1-c1cc(C)ncc1C(=O)Nc1nnc(O[C@H]2CC[C@](C)(O)CC2)s1] |
| 141 | 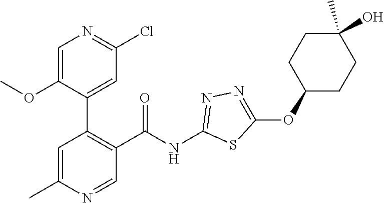 [SMILES: COc1cnc(Cl)cc1-c1cc(C)ncc1C(=O)Nc1nnc(O[C@H]2CC[C@@](C)(O)CC2)s1] |
| 142 | 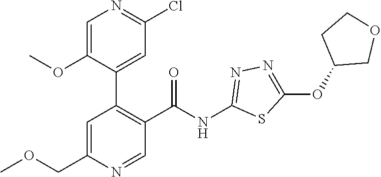 [SMILES: COCc1cc(-c2cc(Cl)ncc2OC)c(C(=O)Nc2nnc(O[C@@H]3CCOC3)s2)cn1] |
| 143 | 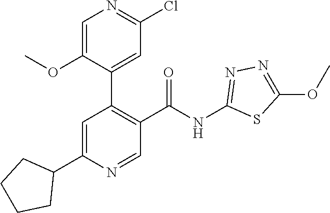 [SMILES: COc1nnc(NC(=O)c2cnc(C3CCCC3)cc2-c2cc(Cl)ncc2OC)s1] |
| 144 | 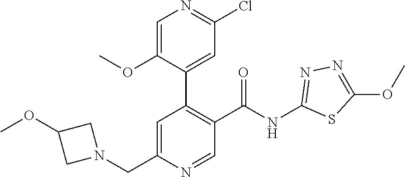 |
| 145 | 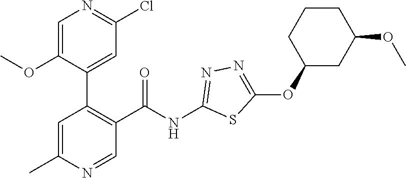 |
| 146 | 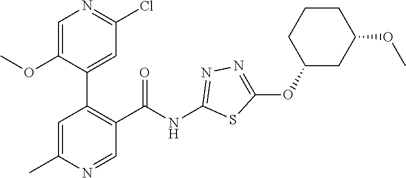 |
| 147 | 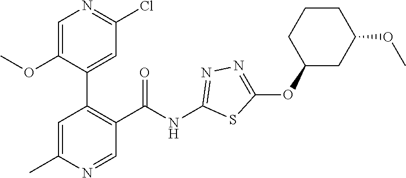 |
| 148 | 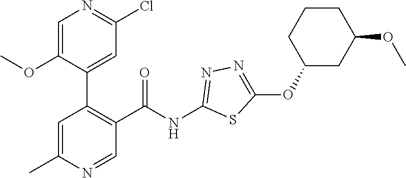 |
| 149 | 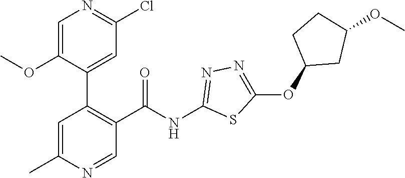 |
| 150 | 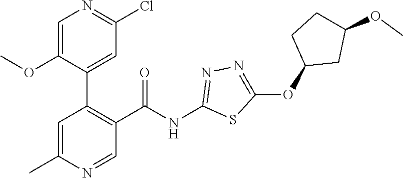 |
| 151 | 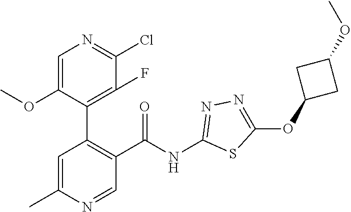 [SMILES: COc1cnc(Cl)c(F)c1-c1cc(C)ncc1C(=O)Nc1nnc(O[C@H]2C[C@H](OC)C2)s1] |
| 152 | 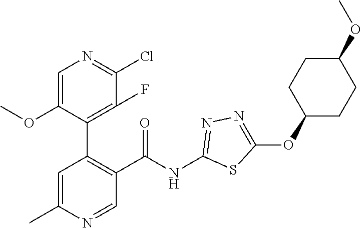 [SMILES: COc1cnc(Cl)c(F)c1-c1cc(C)ncc1C(=O)Nc1nnc(O[C@H]2CC[C@@H](OC)CC2)s1] |
| 153 | 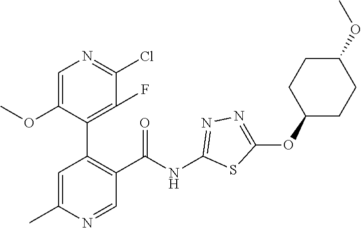 [SMILES: COc1cnc(Cl)c(F)c1-c1cc(C)ncc1C(=O)Nc1nnc(O[C@H]2CC[C@H](OC)CC2)s1] |
| 154 | 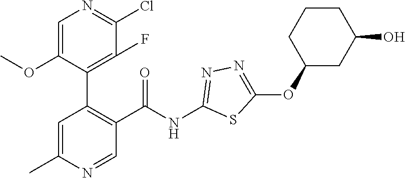 [SMILES: COc1cnc(Cl)c(F)c1-c1cc(C)ncc1C(=O)Nc1nnc(O[C@H]2CCC[C@@H](O)C2)s1] |
| 155 | 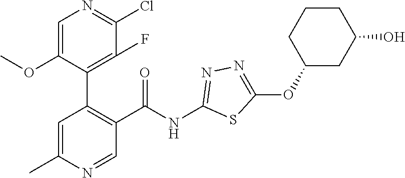 [SMILES: COc1cnc(Cl)c(F)c1-c1cc(C)ncc1C(=O)Nc1nnc(O[C@@H]2CCC[C@H](O)C2)s1] |
| 156 | 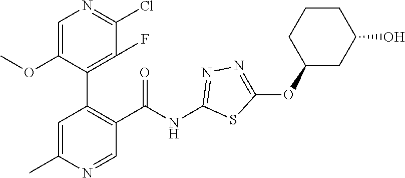 [SMILES: COc1cnc(Cl)c(F)c1-c1cc(C)ncc1C(=O)Nc1nnc(O[C@H]2CCC[C@H](O)C2)s1] |
| 157 | 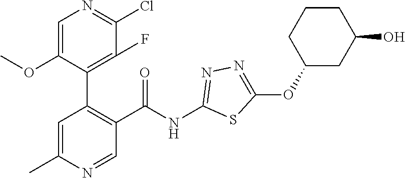 [SMILES: COc1cnc(Cl)c(F)c1-c1cc(C)ncc1C(=O)Nc1nnc(O[C@@H]2CCC[C@@H](O)C2)s1] |
| 158 | 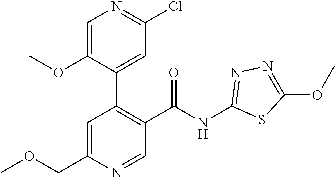 |
| 159 | 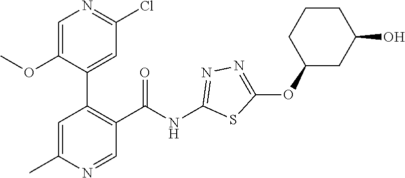 [SMILES: COc1cnc(Cl)cc1-c1cc(C)ncc1C(=O)Nc1nnc(O[C@H]2CCC[C@@H](O)C2)s1] |
| 160 | 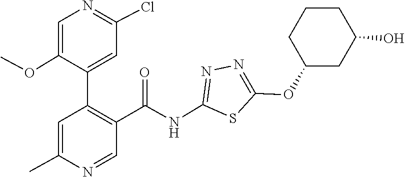 [SMILES: COc1cnc(Cl)cc1-c1cc(C)ncc1C(=O)Nc1nnc(O[C@@H]2CCC[C@H](O)C2)s1] |
| 161 | 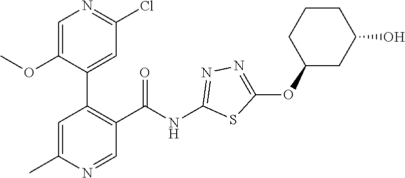 [SMILES: COc1cnc(Cl)cc1-c1cc(C)ncc1C(=O)Nc1nnc(O[C@H]2CCC[C@H](O)C2)s1] |
| 162 | 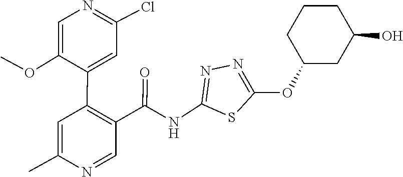 [SMILES: COc1cnc(Cl)cc1-c1cc(C)ncc1C(=O)Nc1nnc(O[C@@H]2CCC[C@@H](O)C2)s1] |
| 163 | 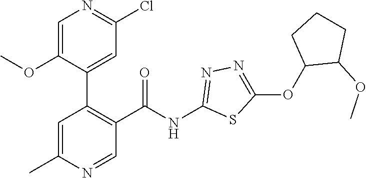 [SMILES: COc1cnc(Cl)cc1-c1cc(C)ncc1C(=O)Nc1nnc(OC2CCCC2OC)s1] |
| 164 | 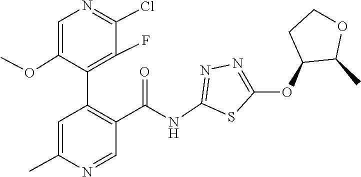 |
| 165 | 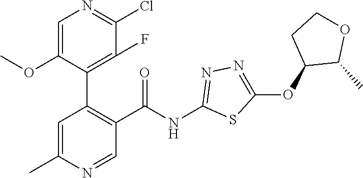 |
| 166 | 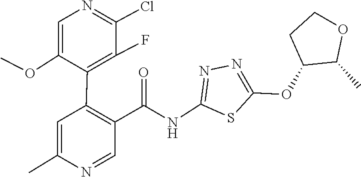 |
| 167 | 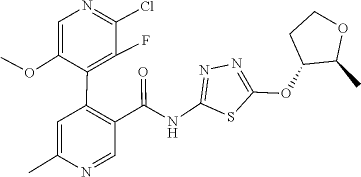 |
| 168 | 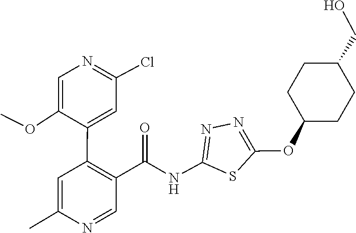 [SMILES: COc1cnc(Cl)cc1-c1cc(C)ncc1C(=O)Nc1nnc(O[C@H]2CC[C@H](CO)CC2)s1] |
| 169 | 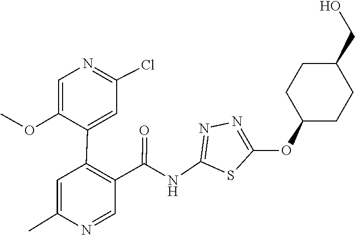 [SMILES: COc1cnc(Cl)cc1-c1cc(C)ncc1C(=O)Nc1nnc(O[C@H]2CC[C@@H](CO)CC2)s1] |
| 170 | 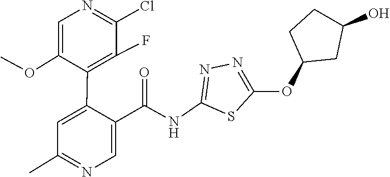 [SMILES: COc1cnc(Cl)c(F)c1-c1cc(C)ncc1C(=O)Nc1nnc(O[C@H]2CC[C@@H](O)C2)s1] |
| 171 | 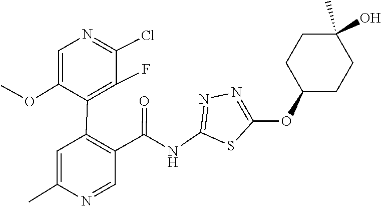 [SMILES: COc1cnc(Cl)c(F)c1-c1cc(C)ncc1C(=O)Nc1nnc(O[C@H]2CC[C@@](C)(O)CC2)s1] |
| 172 | 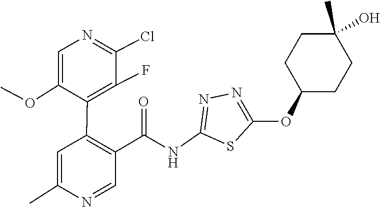 [SMILES: COc1cnc(Cl)c(F)c1-c1cc(C)ncc1C(=O)Nc1nnc(O[C@H]2CC[C@](C)(O)CC2)s1] |
| 173 | 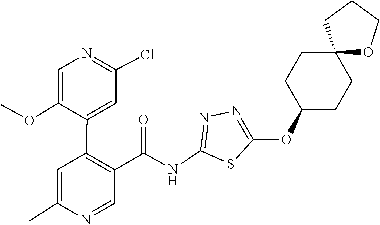 [SMILES: COc1cnc(Cl)cc1-c1cc(C)ncc1C(=O)Nc1nnc(O[C@H]2CC[C@]3(CCCO3)CC2)s1] |
| 174 | 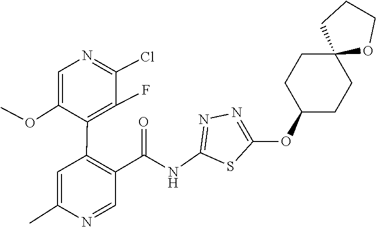 [SMILES: COc1cnc(Cl)c(F)c1-c1cc(C)ncc1C(=O)Nc1nnc(O[C@H]2CC[C@]3(CCCO3)CC2)s1] |
| 175 | 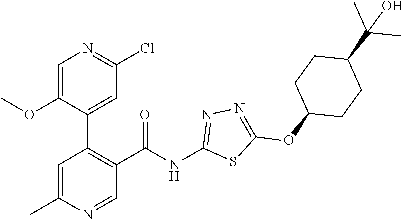 [SMILES: COc1cnc(Cl)cc1-c1cc(C)ncc1C(=O)Nc1nnc(O[C@H]2CC[C@@H](C(C)(C)O)CC2)s1] |
| 176 | 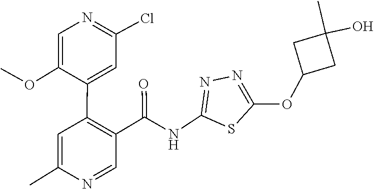 [SMILES: COc1cnc(Cl)cc1-c1cc(C)ncc1C(=O)Nc1nnc(OC2CC(C)(O)C2)s1] |
| 177 | 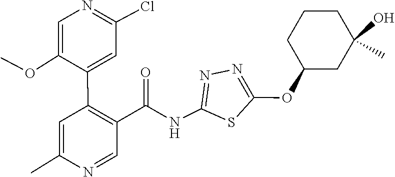 |
| | Racemate |
| 178 | 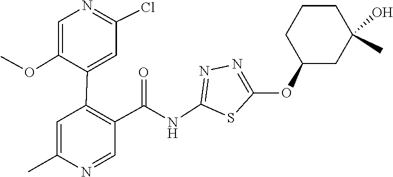 |
| | Racemate |
| 179 | 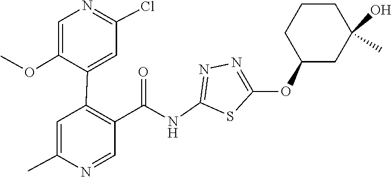 |
| 180 | 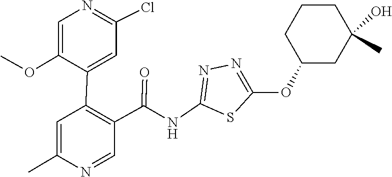 |
| 181 | 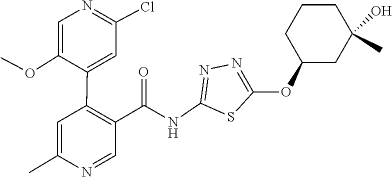 |
| 182 | 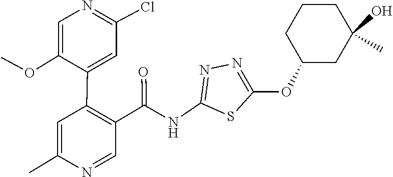 |
| 183 | 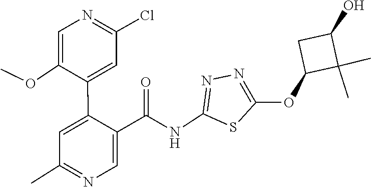 |
| 184 | 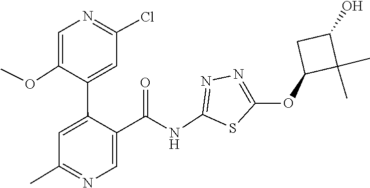 |
| 185 | 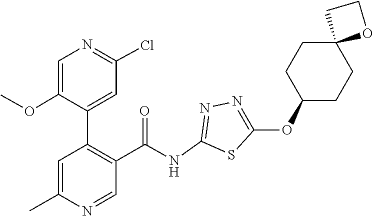 [SMILES: COc1cnc(Cl)cc1-c1cc(C)ncc1C(=O)Nc1nnc(O[C@H]2CC[C@]3(CCO3)CC2)s1] |
| 186 | 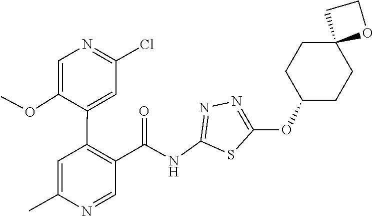 [SMILES: COc1cnc(Cl)cc1-c1cc(C)ncc1C(=O)Nc1nnc(O[C@H]2CC[C@@]3(CCO3)CC2)s1] |
| 187 | 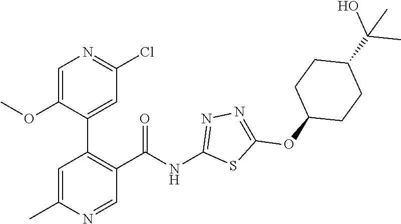 [SMILES: COc1cnc(Cl)cc1-c1cc(C)ncc1C(=O)Nc1nnc(O[C@H]2CC[C@H](C(C)(C)O)CC2)s1] |
| 188 | 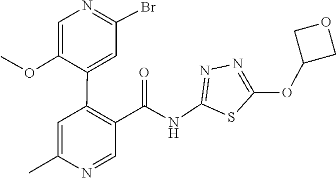 [SMILES: COc1cnc(Br)cc1-c1cc(C)ncc1C(=O)Nc1nnc(OC2COC2)s1] |
| 189 | 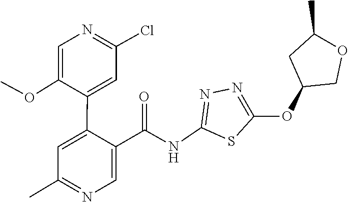 [SMILES: COc1cnc(Cl)cc1-c1cc(C)ncc1C(=O)Nc1nnc(O[C@@H]2CO[C@H](C)C2)s1] |
| 190 | 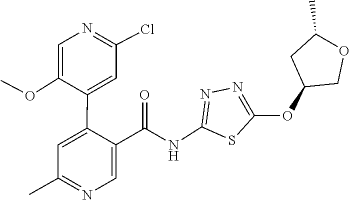 [SMILES: COc1cnc(Cl)cc1-c1cc(C)ncc1C(=O)Nc1nnc(O[C@@H]2CO[C@@H](C)C2)s1] |
| 191 | 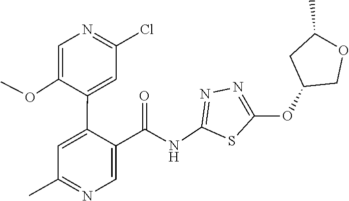 [SMILES: COc1cnc(Cl)cc1-c1cc(C)ncc1C(=O)Nc1nnc(O[C@H]2CO[C@@H](C)C2)s1] |
| 192 | 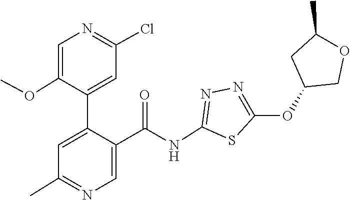 [SMILES: COc1cnc(Cl)cc1-c1cc(C)ncc1C(=O)Nc1nnc(O[C@H]2CO[C@H](C)C2)s1] |
| 193 | 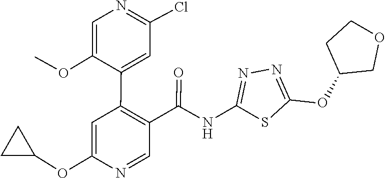 [SMILES: COc1cnc(Cl)cc1-c1cc(OC2CC2)ncc1C(=O)Nc1nnc(O[C@@H]2CCOC2)s1] |
| 194 | 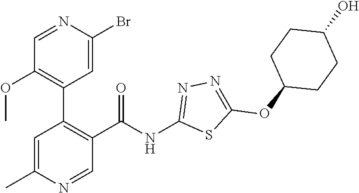 [SMILES: COc1cnc(Br)cc1-c1cc(C)ncc1C(=O)Nc1nnc(O[C@H]2CC[C@H](O)CC2)s1] |
| 195 | 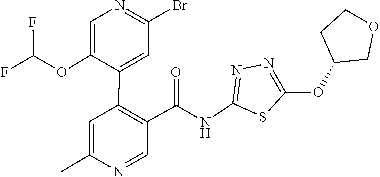 [SMILES: Cc1cc(-c2cc(Br)ncc2OC(F)F)c(C(=O)Nc2nnc(O[C@@H]3CCOC3)s2)cn1] |
| 196 | 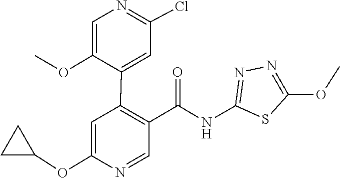 [SMILES: COc1nnc(NC(=O)c2cnc(OC3CC3)cc2-c2cc(Cl)ncc2OC)s1] |
| 197 | 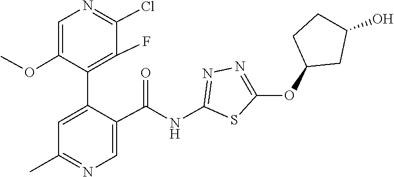 [SMILES: COc1cnc(Cl)c(F)c1-c1cc(C)ncc1C(=O)Nc1nnc(O[C@H]2CC[C@H](O)C2)s1] |
| 198 | 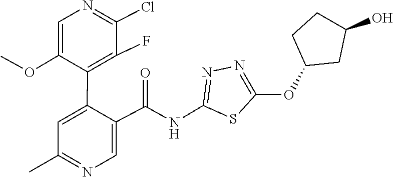 [SMILES: COc1cnc(Cl)c(F)c1-c1cc(C)ncc1C(=O)Nc1nnc(O[C@@H]2CC[C@@H](O)C2)s1] |
| 199 | 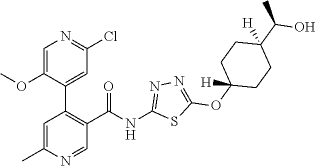 [SMILES: COc1cnc(Cl)cc1-c1cc(C)ncc1C(=O)Nc1nnc(O[C@H]2CC[C@H]([C@@H](C)O)CC2)s1] |
| 200 | 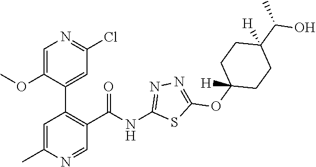 [SMILES: COc1cnc(Cl)cc1-c1cc(C)ncc1C(=O)Nc1nnc(O[C@H]2CC[C@H]([C@H](C)O)CC2)s1] |
| 201 | 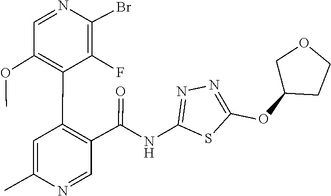 [SMILES: COc1cnc(Br)c(F)c1-c1cc(C)ncc1C(=O)Nc1nnc(O[C@@H]2CCOC2)s1] |
| 202 | 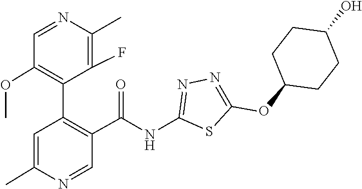 [SMILES: COc1cnc(C)c(F)c1-c1cc(C)ncc1C(=O)Nc1nnc(O[C@H]2CC[C@H](O)CC2)s1] |
| 203 | 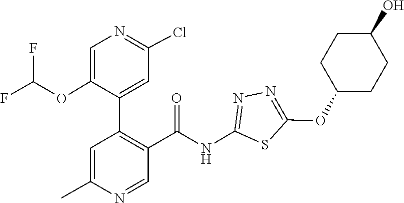 [SMILES: Cc1cc(-c2cc(Cl)ncc2OC(F)F)c(C(=O)Nc2nnc(O[C@H]3CC[C@H](O)CC3)s2)cn1] |
| 204 | 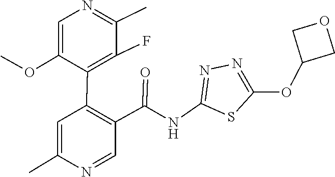 [SMILES: COc1cnc(C)c(F)c1-c1cc(C)ncc1C(=O)Nc1nnc(OC2COC2)s1] |
| 205 | 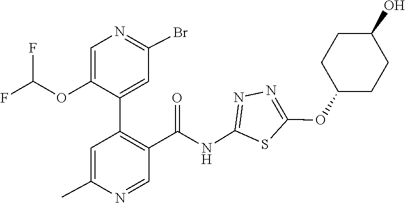 [SMILES: Cc1cc(-c2cc(Br)ncc2OC(F)F)c(C(=O)Nc2nnc(O[C@H]3CC[C@H](O)CC3)s2)cn1] |
| 206 | 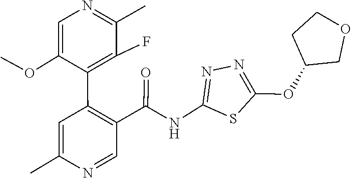 [SMILES: COc1cnc(C)c(F)c1-c1cc(C)ncc1C(=O)Nc1nnc(O[C@@H]2CCOC2)s1] |
| 207 | 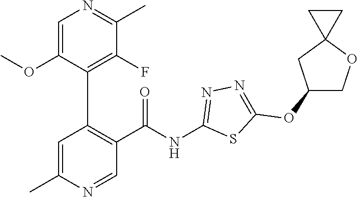 [SMILES: COc1cnc(C)c(F)c1-c1cc(C)ncc1C(=O)Nc1nnc(O[C@@H]2COC3(CC3)C2)s1] |
| 208 | 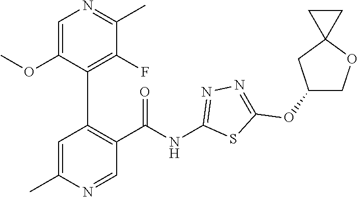 [SMILES: COc1cnc(C)c(F)c1-c1cc(C)ncc1C(=O)Nc1nnc(O[C@H]2COC3(CC3)C2)s1] |
| 209 | 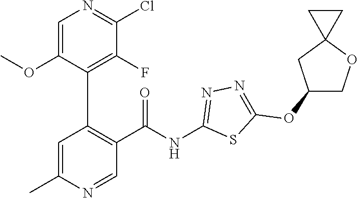 [SMILES: COc1cnc(Cl)c(F)c1-c1cc(C)ncc1C(=O)Nc1nnc(O[C@@H]2COC3(CC3)C2)s1] |
| 210 | 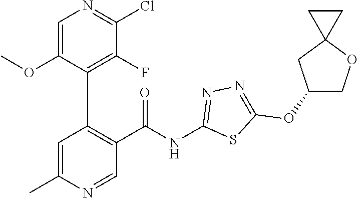 [SMILES: COc1cnc(Cl)c(F)c1-c1cc(C)ncc1C(=O)Nc1nnc(O[C@H]2COC3(CC3)C2)s1] |
| 211 | 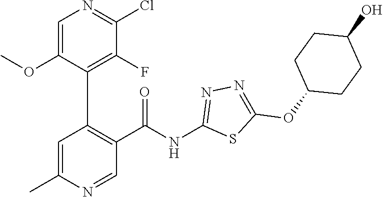 [SMILES: COc1cnc(Cl)c(F)c1-c1cc(C)ncc1C(=O)Nc1nnc(O[C@H]2CC[C@H](O)CC2)s1] |
| 212 | 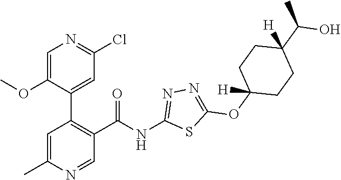 [SMILES: COc1cnc(Cl)cc1-c1cc(C)ncc1C(=O)Nc1nnc(O[C@H]2CC[C@@H]([C@@H](C)O)CC2)s1] |
| 213 | 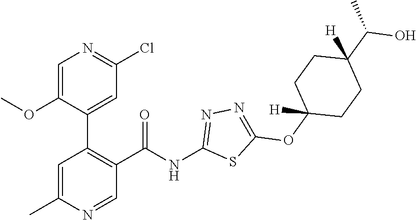 [SMILES: COc1cnc(Cl)cc1-c1cc(C)ncc1C(=O)Nc1nnc(O[C@H]2CC[C@@H]([C@H](C)O)CC2)s1] |
| 214 | 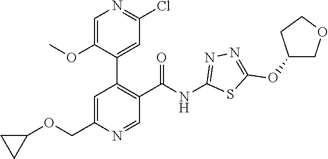 [SMILES: COc1cnc(Cl)cc1-c1cc(COC2CC2)ncc1C(=O)Nc1nnc(O[C@@H]2CCOC2)s1] |
| 215 | 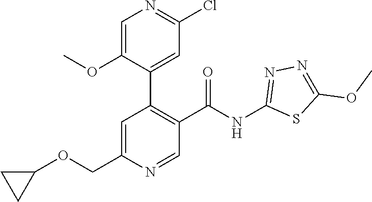 [SMILES: COc1nnc(NC(=O)c2cnc(COC3CC3)cc2-c2cc(Cl)ncc2OC)s1] |
| 216 | 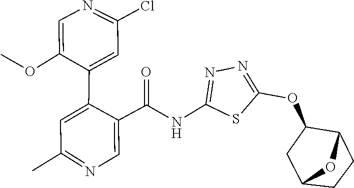 [SMILES: COc1cnc(Cl)cc1-c1cc(C)ncc1C(=O)Nc1nnc(O[C@@H]2C[C@H]3CC[C@@H]2O3)s1] |
| 217 | 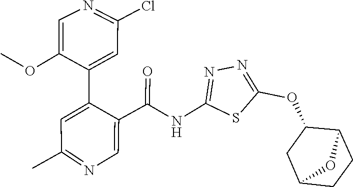 [SMILES: COc1cnc(Cl)cc1-c1cc(C)ncc1C(=O)Nc1nnc(O[C@H]2C[C@@H]3CC[C@H]2O3)s1] |
| 218 | 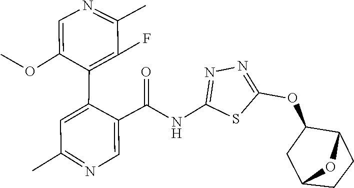 [SMILES: COc1cnc(C)c(F)c1-c1cc(C)ncc1C(=O)Nc1nnc(O[C@@H]2C[C@H]3CC[C@@H]2O3)s1] |
| 219 | 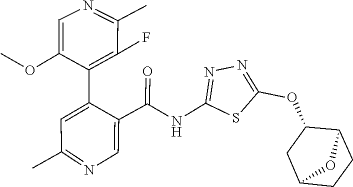 [SMILES: COc1cnc(C)c(F)c1-c1cc(C)ncc1C(=O)Nc1nnc(O[C@H]2C[C@@H]3CC[C@H]2O3)s1] |
| 220 | 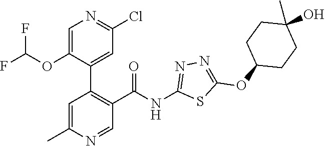 [SMILES: Cc1cc(-c2cc(Cl)ncc2OC(F)F)c(C(=O)Nc2nnc(O[C@H]3CC[C@@](C)(O)CC3)s2)cn1] |
| 221 | 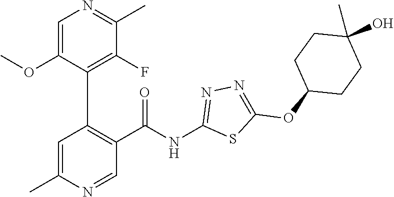 [SMILES: COc1cnc(C)c(F)c1-c1cc(C)ncc1C(=O)Nc1nnc(O[C@H]2CC[C@@](C)(O)CC2)s1] |
| 222 | 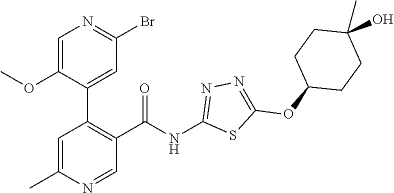 [SMILES: COc1cnc(Br)cc1-c1cc(C)ncc1C(=O)Nc1nnc(O[C@H]2CC[C@@](C)(O)CC2)s1] |
| 223 | 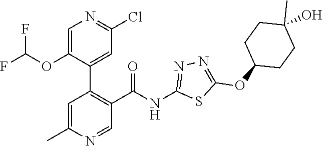 [SMILES: Cc1cc(-c2cc(Cl)ncc2OC(F)F)c(C(=O)Nc2nnc(O[C@H]3CC[C@](C)(O)CC3)s2)cn1] |
| 224 | 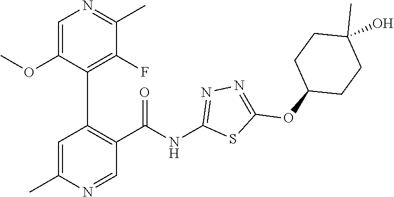 [SMILES: COc1cnc(C)c(F)c1-c1cc(C)ncc1C(=O)Nc1nnc(O[C@H]2CC[C@](C)(O)CC2)s1] |
| 225 | 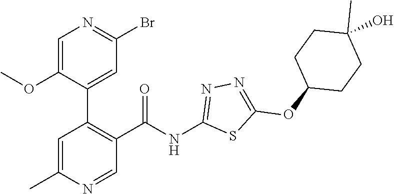 [SMILES: COc1cnc(Br)cc1-c1cc(C)ncc1C(=O)Nc1nnc(O[C@H]2CC[C@](C)(O)CC2)s1] |
| 226 | 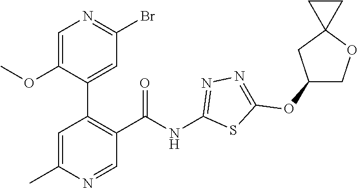 [SMILES: COc1cnc(Br)cc1-c1cc(C)ncc1C(=O)Nc1nnc(O[C@@H]2COC3(CC3)C2)s1] |
| 227 | 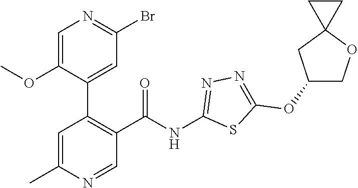 [SMILES: COc1cnc(Br)cc1-c1cc(C)ncc1C(=O)Nc1nnc(O[C@H]2COC3(CC3)C2)s1] |
| 228 | 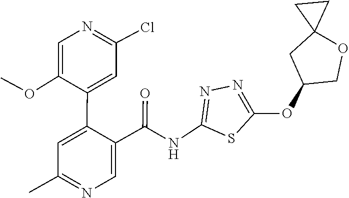 [SMILES: COc1cnc(Cl)cc1-c1cc(C)ncc1C(=O)Nc1nnc(O[C@@H]2COC3(CC3)C2)s1] |
| 229 | 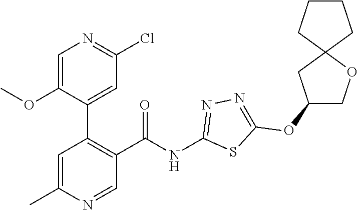 [SMILES: COc1cnc(Cl)cc1-c1cc(C)ncc1C(=O)Nc1nnc(O[C@@H]2COC3(CCCC3)C2)s1] |
| 230 | 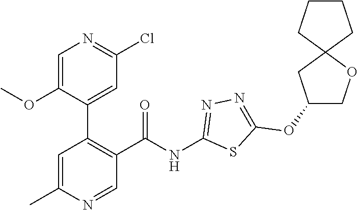 [SMILES: COc1cnc(Cl)cc1-c1cc(C)ncc1C(=O)Nc1nnc(O[C@H]2COC3(CCCC3)C2)s1] |
| 231 | 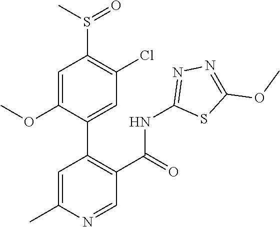 |
| 232 | 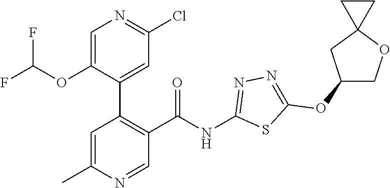 [SMILES: Cc1cc(-c2cc(Cl)ncc2OC(F)F)c(C(=O)Nc2nnc(O[C@@H]3COC4(CC4)C3)s2)cn1] |
| 233 | 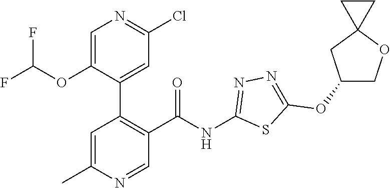 [SMILES: Cc1cc(-c2cc(Cl)ncc2OC(F)F)c(C(=O)Nc2nnc(O[C@H]3COC4(CC4)C3)s2)cn1] |
| 234 | 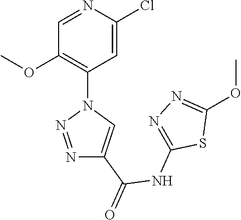 |
| 235 | 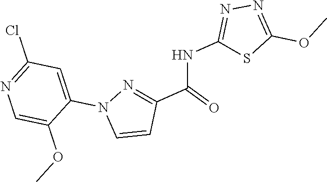 [SMILES: COc1nnc(NC(=O)c2ccn(-c3cc(Cl)ncc3OC)n2)s1] |

Additional representative compounds of Formula (I) are listed in Table 3 below:

TABLE 3

| Cpd # | Structure |
|---|---|
| 236 | 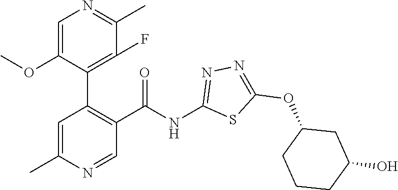 [SMILES: COc1cnc(C)c(F)c1-c1cc(C)ncc1C(=O)Nc1nnc(O[C@H]2CCC[C@@H](O)C2)s1] |
| 237 | 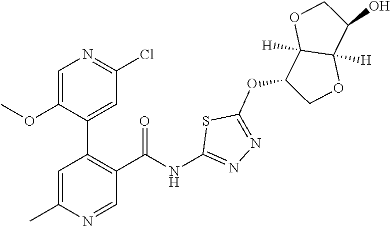 [SMILES: COc1cnc(Cl)cc1-c1cc(C)ncc1C(=O)Nc1nnc(O[C@H]2CO[C@H]3[C@@H]2OC[C@H]3O)s1] |
| 238 | 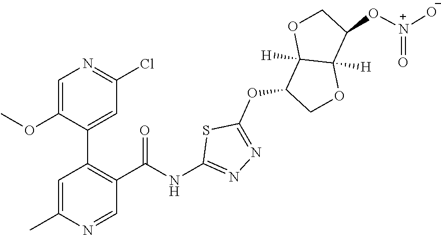 [SMILES: COc1cnc(Cl)cc1-c1cc(C)ncc1C(=O)Nc1nnc(O[C@H]2CO[C@H]3[C@@H]2OC[C@H]3O[N+](=O)[O-])s1] |
| 239 | 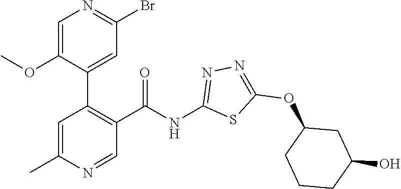 [SMILES: COc1cnc(Br)cc1-c1cc(C)ncc1C(=O)Nc1nnc(O[C@@H]2CCC[C@H](O)C2)s1] |
| 240 | 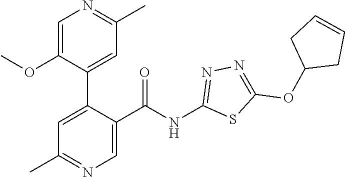 [SMILES: COc1cnc(C)cc1-c1cc(C)ncc1C(=O)Nc1nnc(OC2CC=CC2)s1] |
| 241 | 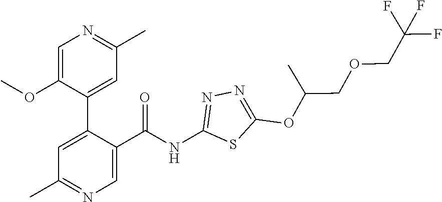 [SMILES: COc1cnc(C)cc1-c1cc(C)ncc1C(=O)Nc1nnc(OC(C)COCC(F)(F)F)s1] |
| 242 | 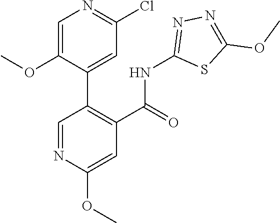 |
| 243 | 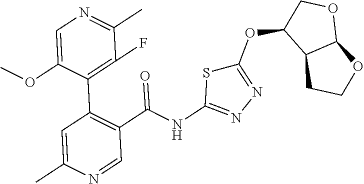 [SMILES: COc1cnc(C)c(F)c1-c1cc(C)ncc1C(=O)Nc1nnc(O[C@H]2CO[C@H]3OCC[C@H]32)s1] |
| 244 | 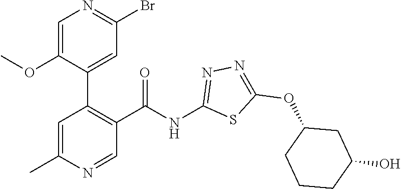 [SMILES: COc1cnc(Br)cc1-c1cc(C)ncc1C(=O)Nc1nnc(O[C@H]2CCC[C@@H](O)C2)s1] |
| 245 | 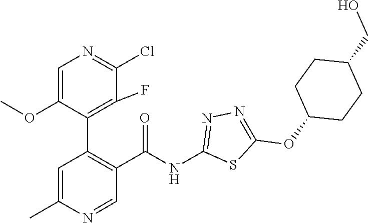 [SMILES: COc1cnc(Cl)c(F)c1-c1cc(C)ncc1C(=O)Nc1nnc(O[C@H]2CC[C@@H](CO)CC2)s1] |
| 246 | 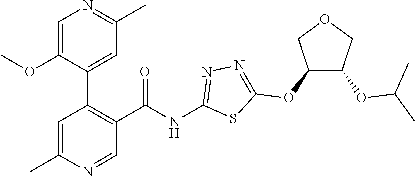 [SMILES: COc1cnc(C)cc1-c1cc(C)ncc1C(=O)Nc1nnc(O[C@H]2COC[C@@H]2OC(C)C)s1] |
| 247 | 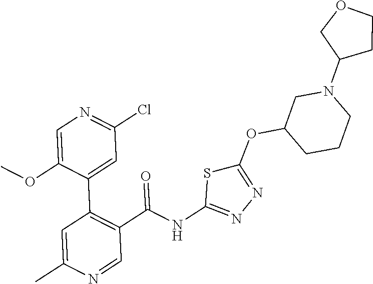 [SMILES: COc1cnc(Cl)cc1-c1cc(C)ncc1C(=O)Nc1nnc(OC2CCCN(C3CCOC3)C2)s1] |
| 248 | 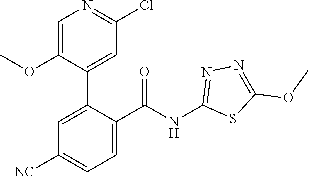 |
| 249 | 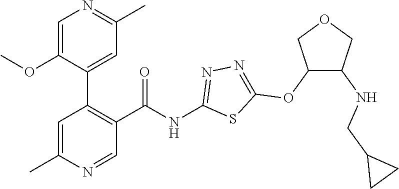 [SMILES: COc1cnc(C)cc1-c1cc(C)ncc1C(=O)Nc1nnc(OC2COCC2NCC2CC2)s1] |
| 250 | 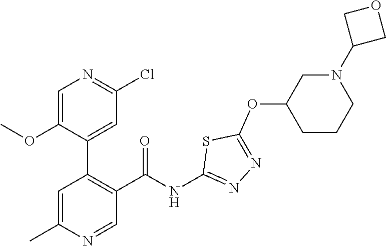 [SMILES: COc1cnc(Cl)cc1-c1cc(C)ncc1C(=O)Nc1nnc(OC2CCCN(C3COC3)C2)s1] |
| 251 | 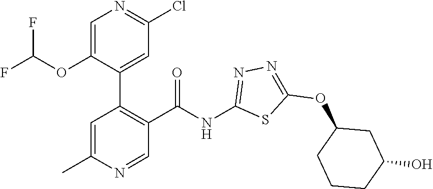 [SMILES: Cc1cc(-c2cc(Cl)ncc2OC(F)F)c(C(=O)Nc2nnc(O[C@@H]3CCC[C@@H](O)C3)s2)cn1] |
| 252 | 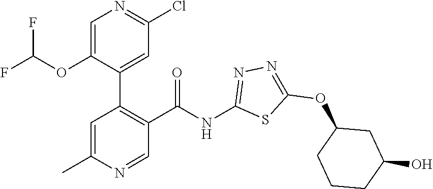 [SMILES: Cc1cc(-c2cc(Cl)ncc2OC(F)F)c(C(=O)Nc2nnc(O[C@@H]3CCC[C@H](O)C3)s2)cn1] |
| 253 | 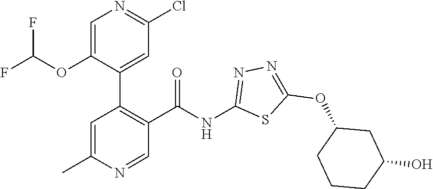 [SMILES: Cc1cc(-c2cc(Cl)ncc2OC(F)F)c(C(=O)Nc2nnc(O[C@H]3CCC[C@@H](O)C3)s2)cn1] |
| 254 | 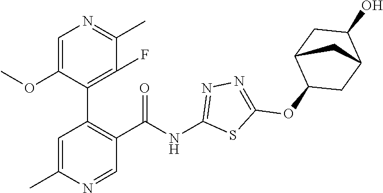 [SMILES: COc1cnc(C)c(F)c1-c1cc(C)ncc1C(=O)Nc1nnc(O[C@@H]2C[C@@H]3C[C@H]2C[C@H]3O)s1] |
| 255 | 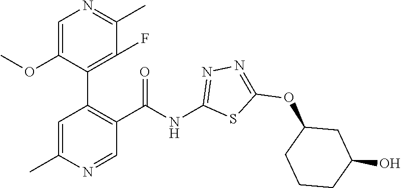 [SMILES: COc1cnc(C)c(F)c1-c1cc(C)ncc1C(=O)Nc1nnc(O[C@@H]2CCC[C@H](O)C2)s1] |
| 256 | 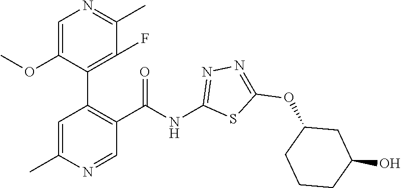 [SMILES: COc1cnc(C)c(F)c1-c1cc(C)ncc1C(=O)Nc1nnc(O[C@H]2CCC[C@H](O)C2)s1] |
| 257 | 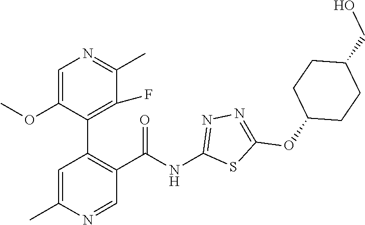 [SMILES: COc1cnc(C)c(F)c1-c1cc(C)ncc1C(=O)Nc1nnc(O[C@H]2CC[C@@H](CO)CC2)s1] |
| 258 | 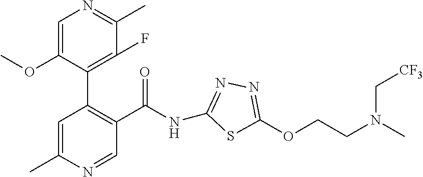 [SMILES: COc1cnc(C)c(F)c1-c1cc(C)ncc1C(=O)Nc1nnc(OCCN(C)CC(F)(F)F)s1] |
| 259 | 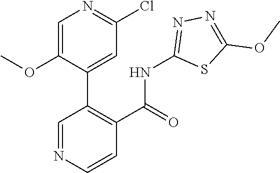 |
| 260 | 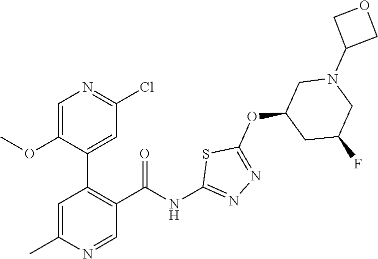 [SMILES: COc1cnc(Cl)cc1-c1cc(C)ncc1C(=O)Nc1nnc(O[C@@H]2C[C@H](F)CN(C3COC3)C2)s1] |
| 261 | 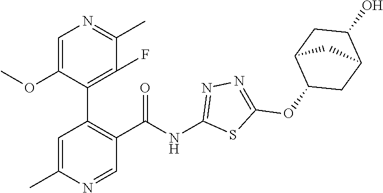 [SMILES: COc1cnc(C)c(F)c1-c1cc(C)ncc1C(=O)Nc1nnc(O[C@H]2C[C@H]3C[C@@H]2C[C@@H]3O)s1] |
| 262 | 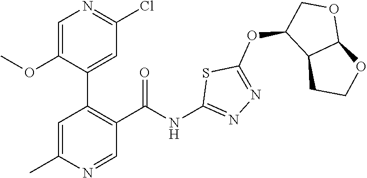 [SMILES: COc1cnc(Cl)cc1-c1cc(C)ncc1C(=O)Nc1nnc(O[C@H]2CO[C@H]3OCC[C@H]32)s1] |
| 263 | 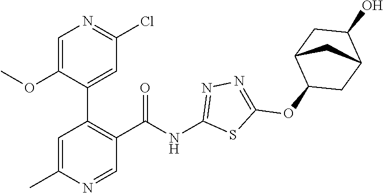 [SMILES: COc1cnc(Cl)cc1-c1cc(C)ncc1C(=O)Nc1nnc(O[C@@H]2C[C@@H]3C[C@H]2C[C@H]3O)s1] |
| 264 | 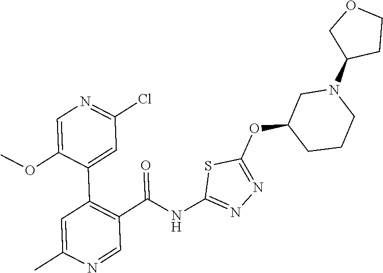 [SMILES: COc1cnc(Cl)cc1-c1cc(C)ncc1C(=O)Nc1nnc(O[C@@H]2CCCN([C@@H]3CCOC3)C2)s1] |
| 265 | 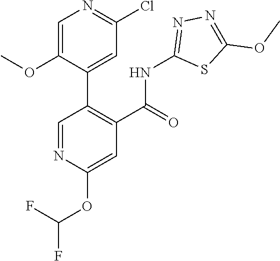 |
| 266 | 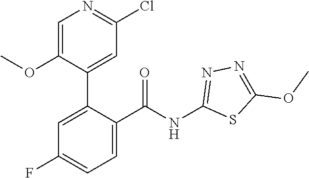 |
| 267 | 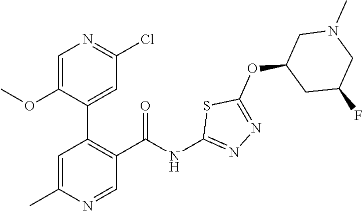 [SMILES: COc1cnc(Cl)cc1-c1cc(C)ncc1C(=O)Nc1nnc(O[C@@H]2C[C@H](F)CN(C)C2)s1] |
| 268 | 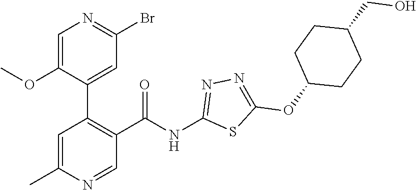 [SMILES: COc1cnc(Br)cc1-c1cc(C)ncc1C(=O)Nc1nnc(O[C@H]2CC[C@@H](CO)CC2)s1] |
| 269 | 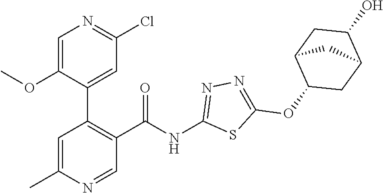 [SMILES: COc1cnc(Cl)cc1-c1cc(C)ncc1C(=O)Nc1nnc(O[C@H]2C[C@H]3C[C@@H]2C[C@@H]3O)s1] |
| 270 | 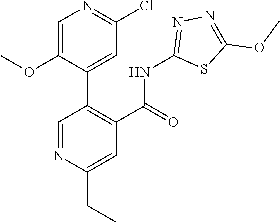 |
| 271 | 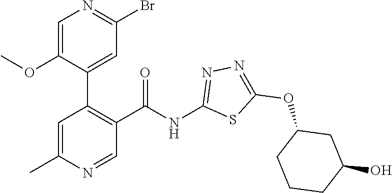 [SMILES: COc1cnc(Br)cc1-c1cc(C)ncc1C(=O)Nc1nnc(O[C@H]2CCC[C@H](O)C2)s1] |
| 272 | 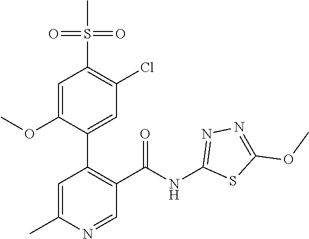 |
| 273 | 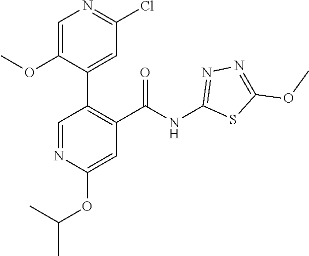 |
| 274 | 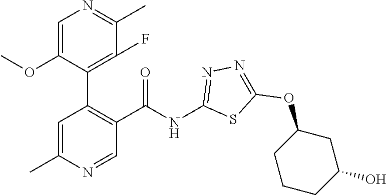 [SMILES: COc1cnc(C)c(F)c1-c1cc(C)ncc1C(=O)Nc1nnc(O[C@@H]2CCC[C@@H](O)C2)s1] |
| 275 | 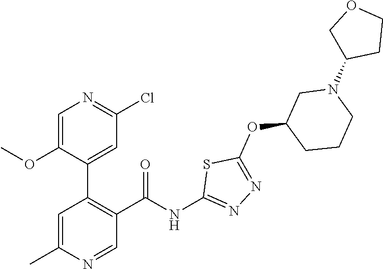 [SMILES: COc1cnc(Cl)cc1-c1cc(C)ncc1C(=O)Nc1nnc(O[C@@H]2CCCN([C@H]3CCOC3)C2)s1] |
| 276 | 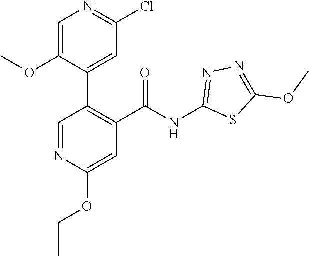 |
| 277 | 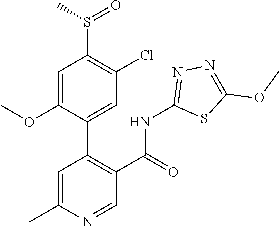 |
| 278 | 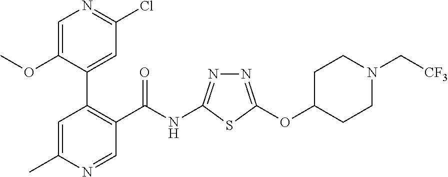 [SMILES: COc1cnc(Cl)cc1-c1cc(C)ncc1C(=O)Nc1nnc(OC2CCN(CC(F)(F)F)CC2)s1] |
| 279 | 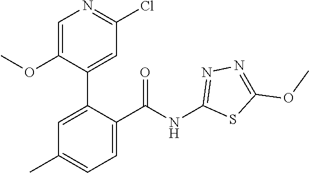 |
| 280 | 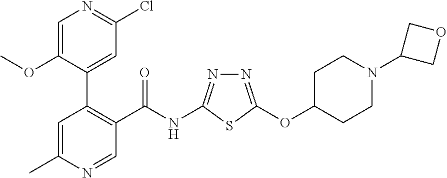 [SMILES: COc1cnc(Cl)cc1-c1cc(C)ncc1C(=O)Nc1nnc(OC2CCN(C3COC3)CC2)s1] |
| 281 | 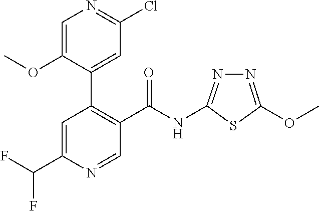 |
| 282 | 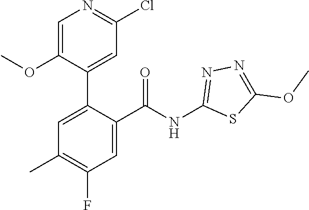 |
| 283 | 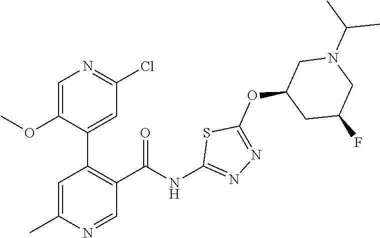 [SMILES: COc1cnc(Cl)cc1-c1cc(C)ncc1C(=O)Nc1nnc(O[C@@H]2C[C@H](F)CN(C(C)C)C2)s1] |
| 284 | 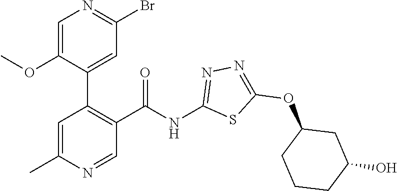 [SMILES: COc1cnc(Br)cc1-c1cc(C)ncc1C(=O)Nc1nnc(O[C@@H]2CCC[C@@H](O)C2)s1] |
| 285 | 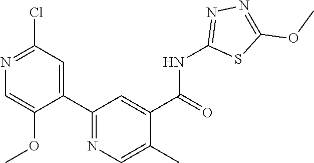 [SMILES: COc1nnc(NC(=O)c2cc(-c3cc(Cl)ncc3OC)ncc2C)s1] |
| 286 | 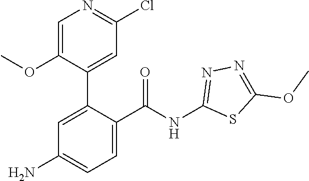 |
| 287 | 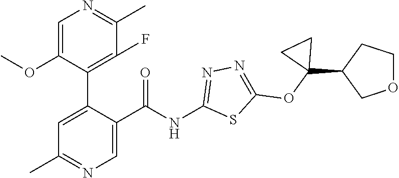 [SMILES: COc1cnc(C)c(F)c1-c1cc(C)ncc1C(=O)Nc1nnc(OC2([C@H]3CCOC3)CC2)s1] |
| 288 | 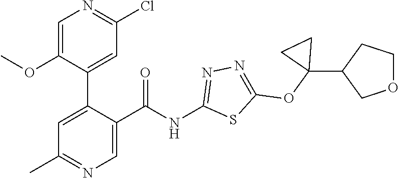 [SMILES: COc1cnc(Cl)cc1-c1cc(C)ncc1C(=O)Nc1nnc(OC2(C3CCOC3)CC2)s1] |
| 289 | 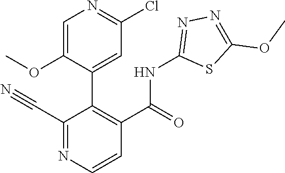 |
| 290 | 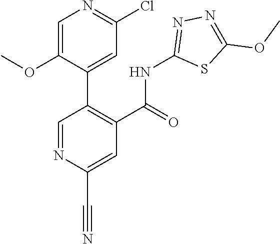 |
| 291 | 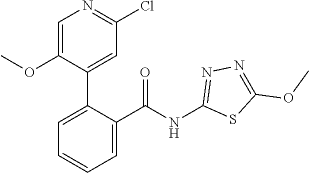 |
| 292 | 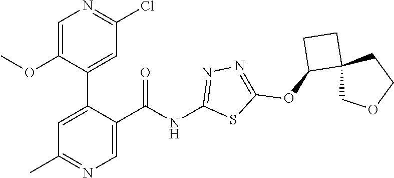 [SMILES: COc1cnc(Cl)cc1-c1cc(C)ncc1C(=O)Nc1nnc(O[C@H]2CC[C@@]23CCOC3)s1] |
| 293 | 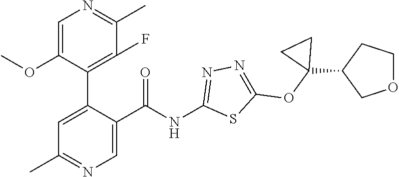 [SMILES: COc1cnc(C)c(F)c1-c1cc(C)ncc1C(=O)Nc1nnc(OC2([C@@H]3CCOC3)CC2)s1] |
| 294 | 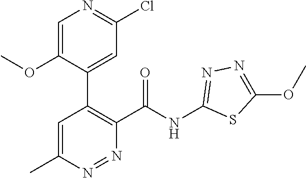 |
| 295 | 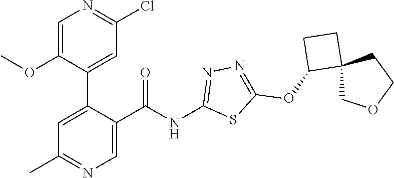 [SMILES: COc1cnc(Cl)cc1-c1cc(C)ncc1C(=O)Nc1nnc(O[C@@H]2CC[C@@]23CCOC3)s1] |
| 296 | 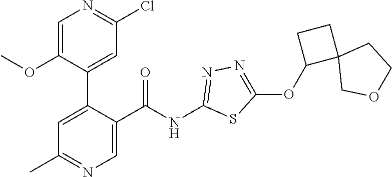 [SMILES: COc1cnc(Cl)cc1-c1cc(C)ncc1C(=O)Nc1nnc(OC2CCC23CCOC3)s1] |
| 297 | 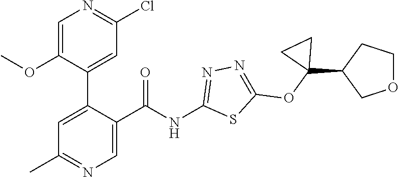 [SMILES: COc1cnc(Cl)cc1-c1cc(C)ncc1C(=O)Nc1nnc(OC2([C@H]3CCOC3)CC2)s1] |
| 298 | 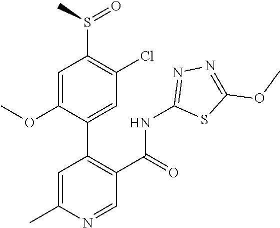 |
| 299 | 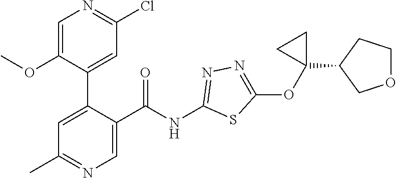 [SMILES: COc1cnc(Cl)cc1-c1cc(C)ncc1C(=O)Nc1nnc(OC2([C@@H]3CCOC3)CC2)s1] |
| 300 | 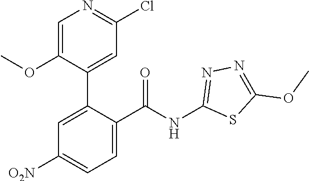 |
| 301 | 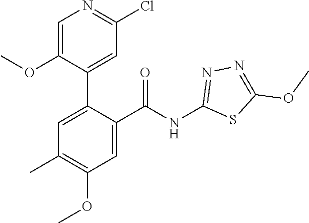 |
| 302 | 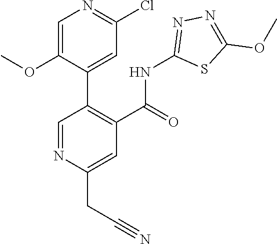 |
| 303 | 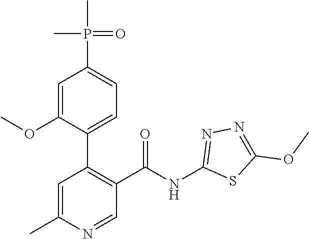 |
| 304 | 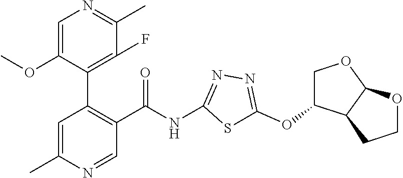 [SMILES: COc1cnc(C)c(F)c1-c1cc(C)ncc1C(=O)Nc1nnc(O[C@@H]2CO[C@H]3OCC[C@H]32)s1] |
| 305 | 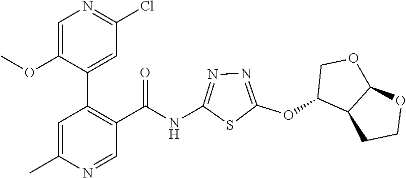 [SMILES: COc1cnc(Cl)cc1-c1cc(C)ncc1C(=O)Nc1nnc(O[C@@H]2CO[C@H]3OCC[C@H]32)s1] |
| 306 | 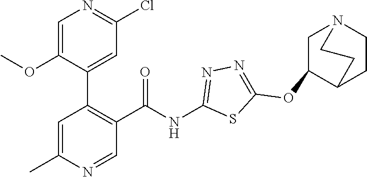 [SMILES: COc1cnc(Cl)cc1-c1cc(C)ncc1C(=O)Nc1nnc(O[C@H]2CN3CCC2CC3)s1] |
| 307 | 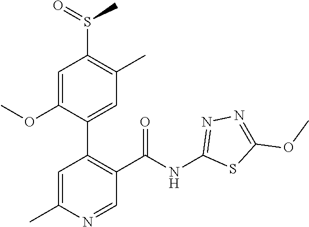 |
| 308 | 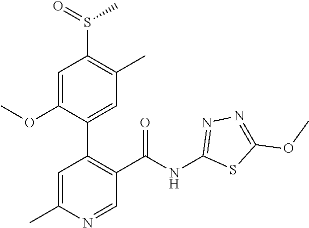 |
| 309 | 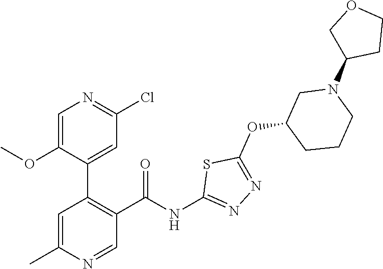 [SMILES: COc1cnc(Cl)cc1-c1cc(C)ncc1C(=O)Nc1nnc(O[C@H]2CCCN([C@@H]3CCOC3)C2)s1] |
| 310 | 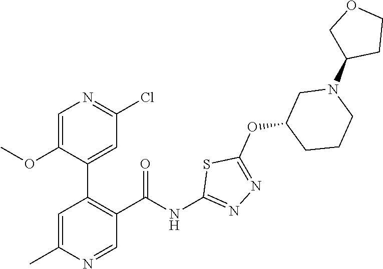 [SMILES: COc1cnc(Cl)cc1-c1cc(C)ncc1C(=O)Nc1nnc(O[C@H]2CCCN([C@@H]3CCOC3)C2)s1] |
| 311 | 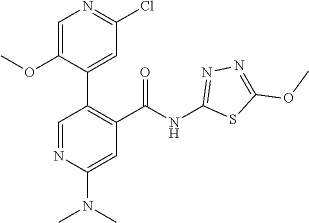 |
| 312 | 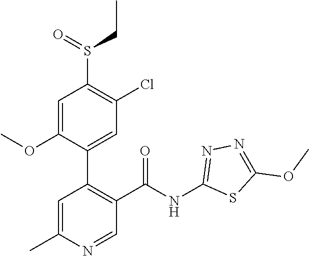 |
| 313 | 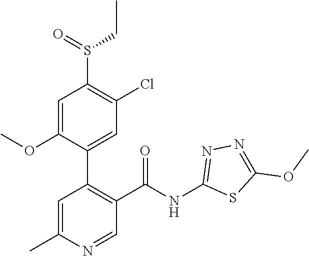 |
| 314 | 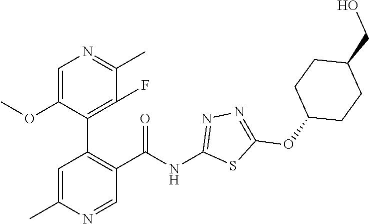 [SMILES: COc1cnc(C)c(F)c1-c1cc(C)ncc1C(=O)Nc1nnc(O[C@H]2CC[C@H](CO)CC2)s1] |
| 315 | 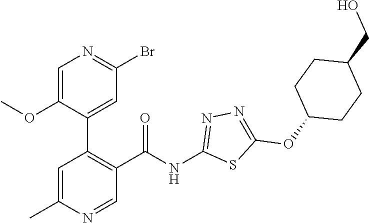 [SMILES: COc1cnc(Br)cc1-c1cc(C)ncc1C(=O)Nc1nnc(O[C@H]2CC[C@H](CO)CC2)s1] |
| 316 | 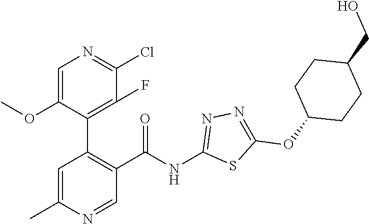 [SMILES: COc1cnc(Cl)c(F)c1-c1cc(C)ncc1C(=O)Nc1nnc(O[C@H]2CC[C@H](CO)CC2)s1] |
| 317 | 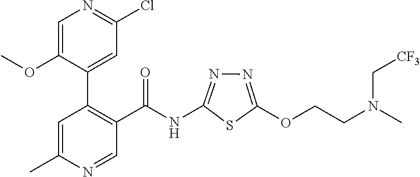 [SMILES: COc1cnc(Cl)cc1-c1cc(C)ncc1C(=O)Nc1nnc(OCCN(C)CC(F)(F)F)s1] |
| 318 | 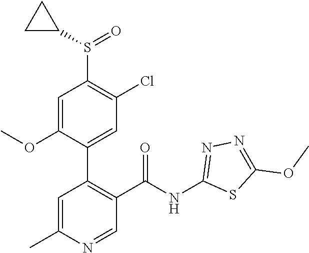 |
| 319 | 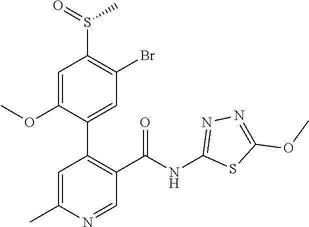 |
| 320 | 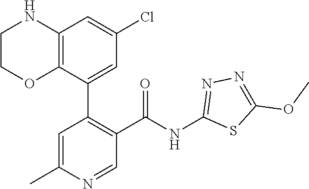 [SMILES: COc1nnc(NC(=O)c2cnc(C)cc2-c2cc(Cl)cc3c2OCCN3)s1] |
| 321 | 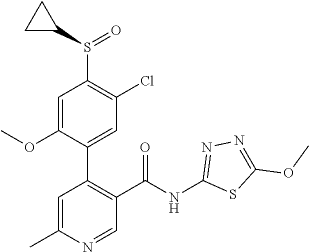 |
| 322 | 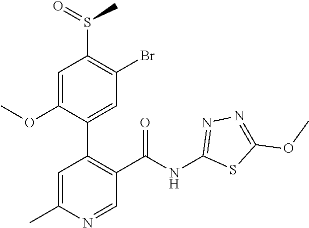 |
| 323 | 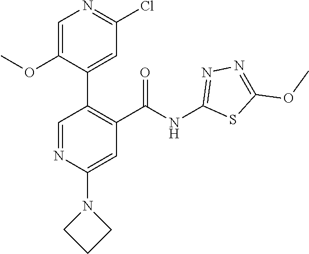 |
| 324 | 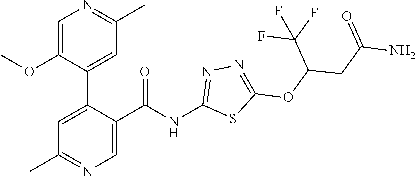 [SMILES: COc1cnc(C)cc1-c1cc(C)ncc1C(=O)Nc1nnc(OC(CC(N)=O)C(F)(F)F)s1] |
| 325 | 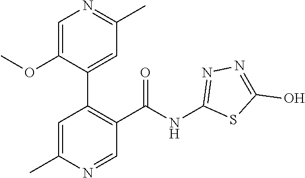 [SMILES: COc1cnc(C)cc1-c1cc(C)ncc1C(=O)Nc1nnc(O)s1] |
| 326 | 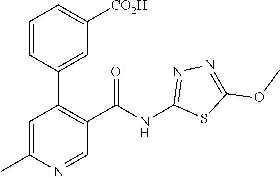 |
| 327 | 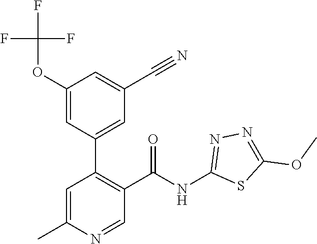 [SMILES: COc1nnc(NC(=O)c2cnc(C)cc2-c2cc(C#N)cc(OC(F)(F)F)c2)s1] |
| 328 | 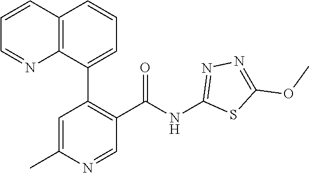 [SMILES: COc1nnc(NC(=O)c2cnc(C)cc2-c2cccc3cccnc23)s1] |
| 329 | 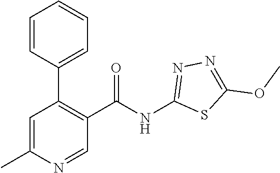 |
| 330 | 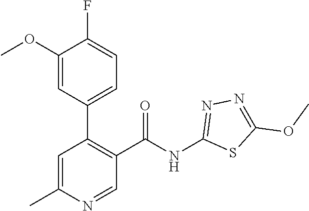 |
| 331 | 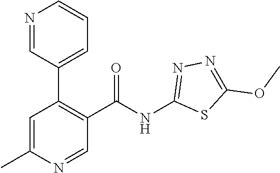 |
| 332 | 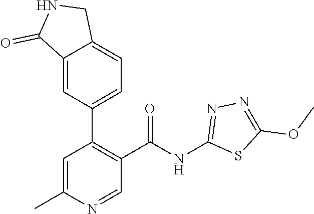 [SMILES: COc1nnc(NC(=O)c2cnc(C)cc2-c2ccc3c(c2)C(=O)NC3)s1] |
| 333 | 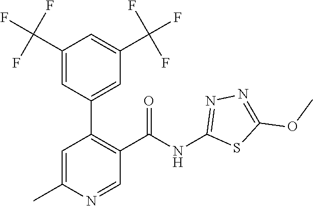 [SMILES: COc1nnc(NC(=O)c2cnc(C)cc2-c2cc(C(F)(F)F)cc(C(F)(F)F)c2)s1] |
| 334 | 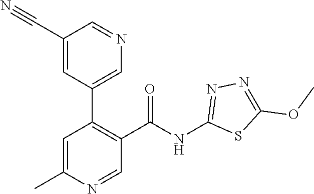 [SMILES: COc1nnc(NC(=O)c2cnc(C)cc2-c2cncc(C#N)c2)s1] |
| 335 | 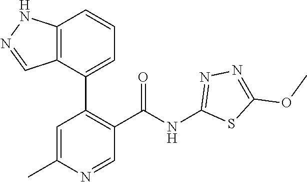 [SMILES: COc1nnc(NC(=O)c2cnc(C)cc2-c2cccc3[nH]ncc23)s1] |
| 336 | 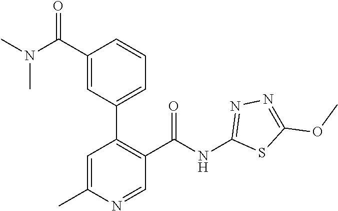 |
| 337 | 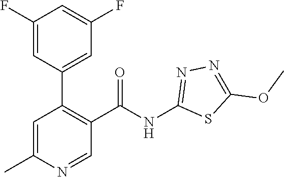 [SMILES: COc1nnc(NC(=O)c2cnc(C)cc2-c2cc(F)cc(F)c2)s1] |
| 338 | 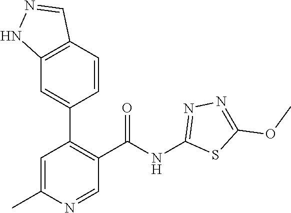 |
| 339 | 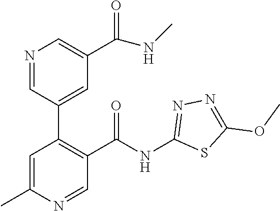 |
| 340 | 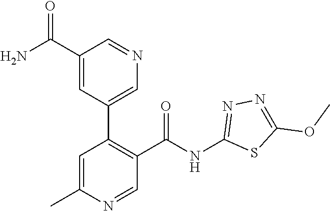 [SMILES: COc1nnc(NC(=O)c2cnc(C)cc2-c2cncc(C(N)=O)c2)s1] |
| 341 | 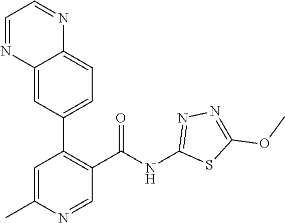 |
| 342 | 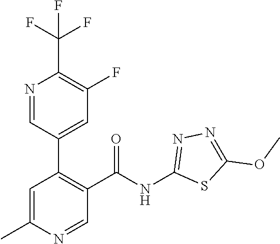 |
| 343 | 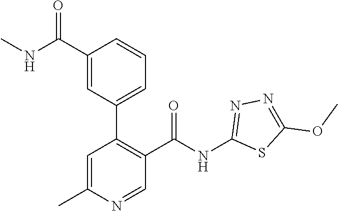 |
| 344 | 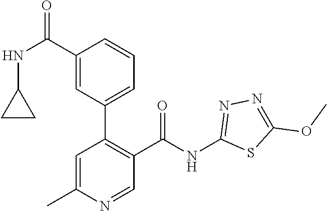 [SMILES: COc1nnc(NC(=O)c2cnc(C)cc2-c2cccc(C(=O)NC3CC3)c2)s1] |
| 345 | 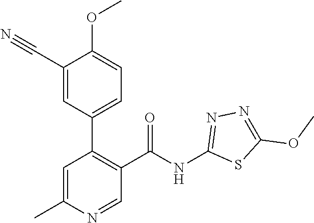 [SMILES: COc1nnc(NC(=O)c2cnc(C)cc2-c2ccc(OC)c(C#N)c2)s1] |
| 346 | 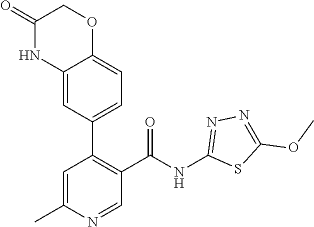 [SMILES: COc1nnc(NC(=O)c2cnc(C)cc2-c2ccc3c(c2)NC(=O)CO3)s1] |
| 347 | 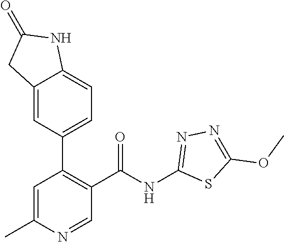 |
| 348 | 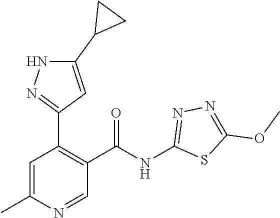 |
| 349 | 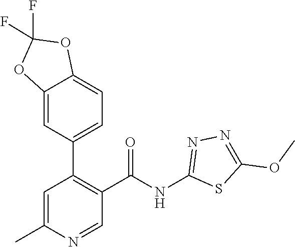 [SMILES: COc1nnc(NC(=O)c2cnc(C)cc2-c2ccc3c(c2)OC(F)(F)O3)s1] |
| 350 | 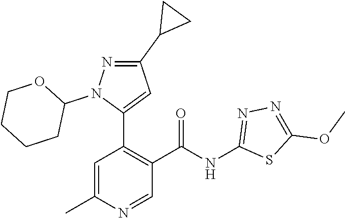 [SMILES: COc1nnc(NC(=O)c2cnc(C)cc2-c2cc(C3CC3)nn2C2CCCCO2)s1] |
| 351 | 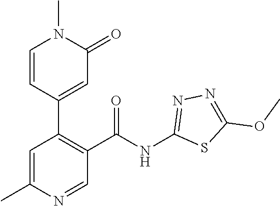 |
| 352 | 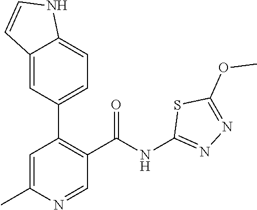 |
| 353 | 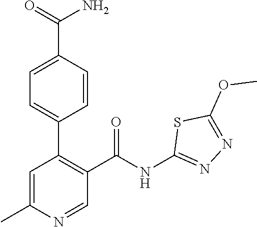 |
| 354 | 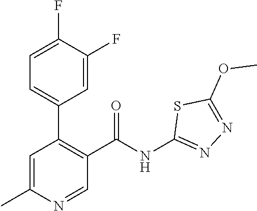 |
| 355 | 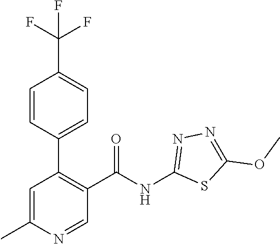 |
| 356 | 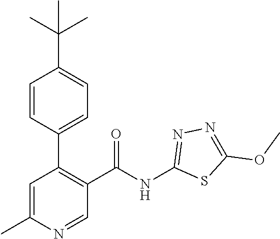 |
| 357 | 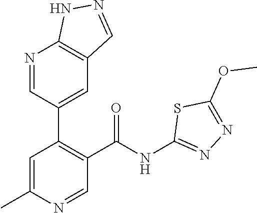 |
| 358 | 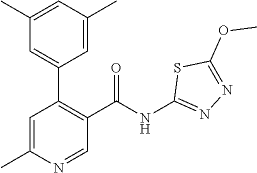 |
| 359 | 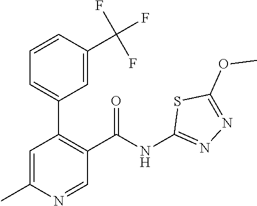 |
| 360 | 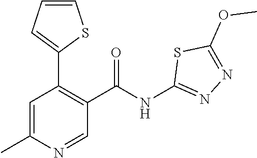 |
| 361 | 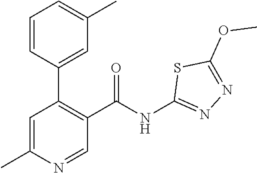 |
| 362 | 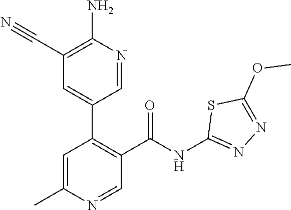 [SMILES: COc1nnc(NC(=O)c2cnc(C)cc2-c2cnc(N)c(C#N)c2)s1] |
| 363 | 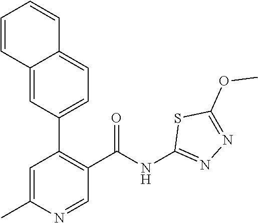 |
| 364 | 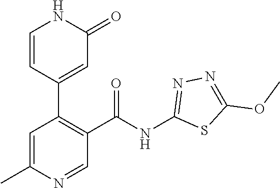 |
| 365 | 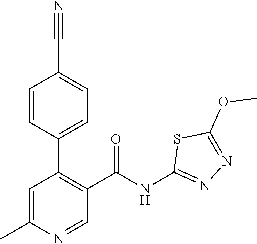 |
| 366 | 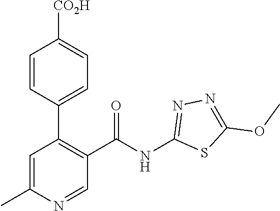 |
| 367 | 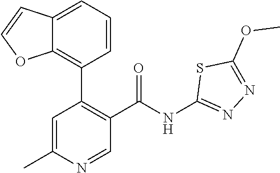 [SMILES: COc1nnc(NC(=O)c2cnc(C)cc2-c2cccc3ccoc23)s1] |
| 368 | 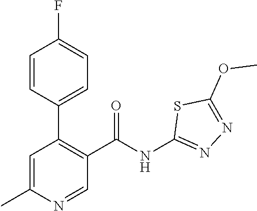 |
| 369 | 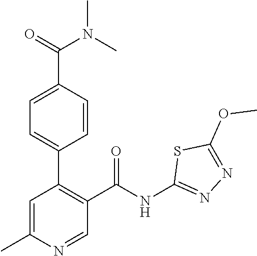 |
| 370 | 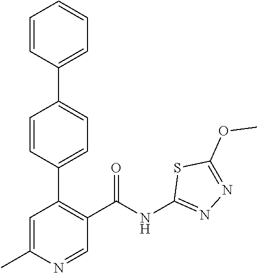 |
| 371 | 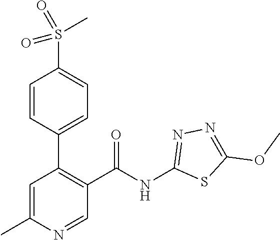 |
| 372 | 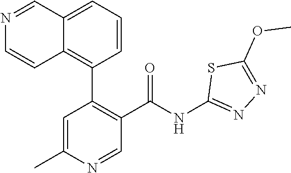 [SMILES: COc1nnc(NC(=O)c2cnc(C)cc2-c2cccc3cnccc23)s1] |
| 373 | 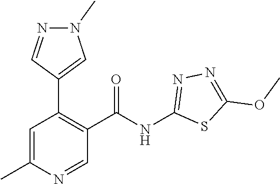 |
| 374 | 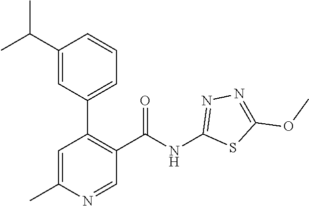 [SMILES: COc1nnc(NC(=O)c2cnc(C)cc2-c2cccc(C(C)C)c2)s1] |
| 375 | 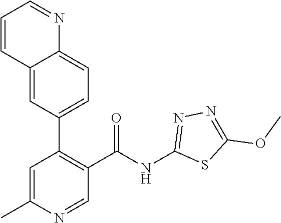 |
| 376 | 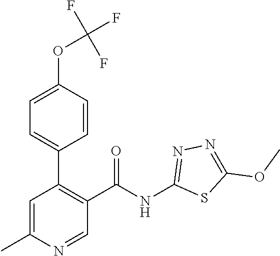 |
| 377 | 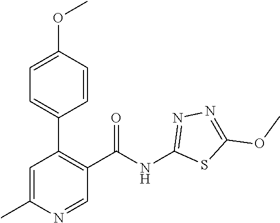 |
| 378 | 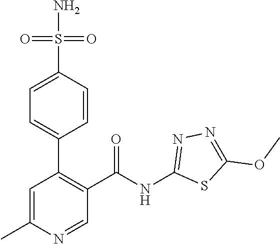 |
| 379 | 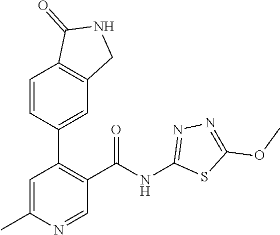 |
| 380 | 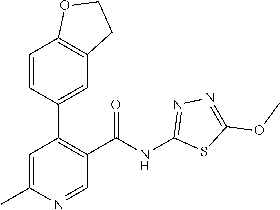 |
| 381 | 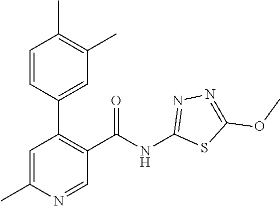 |
| 382 | 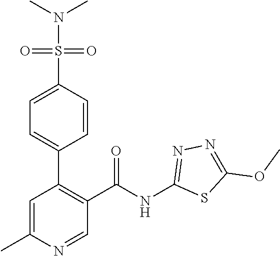 |
| 383 | 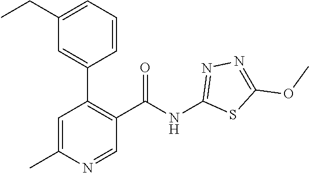 [SMILES: CCc1cccc(-c2cc(C)ncc2C(=O)Nc2nnc(OC)s2)c1] |
| 384 | 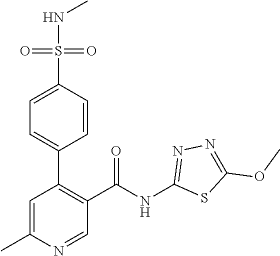 |
| 385 | 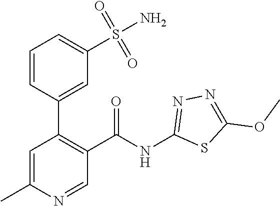 |
| 386 | 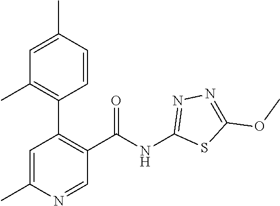 |
| 387 | 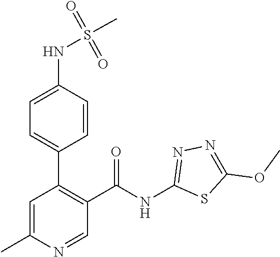 |
| 388 | 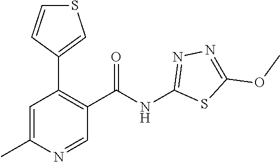 |
| 389 | 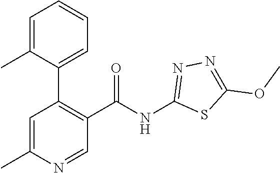 |
| 390 | 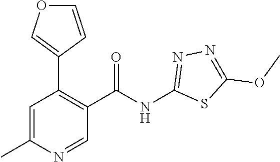 |
| 391 | 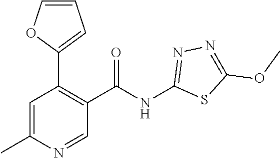 |
| 392 | 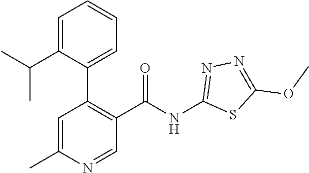 [SMILES: COc1nnc(NC(=O)c2cnc(C)cc2-c2ccccc2C(C)C)s1] |
| 393 | 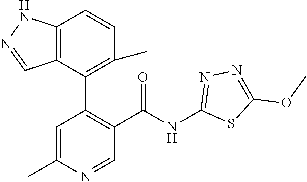 [SMILES: COc1nnc(NC(=O)c2cnc(C)cc2-c2c(C)ccc3[nH]ncc23)s1] |
| 394 | 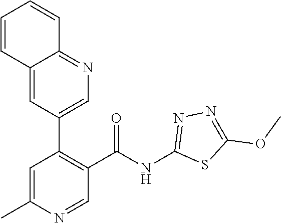 |
| 395 | 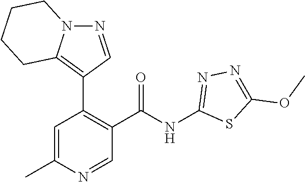 [SMILES: COc1nnc(NC(=O)c2cnc(C)cc2-c2cnn3c2CCCC3)s1] |
| 396 | 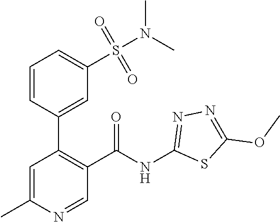 |
| 397 | 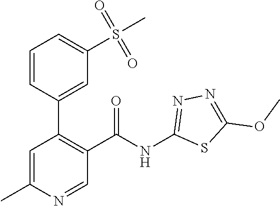 |
| 398 | 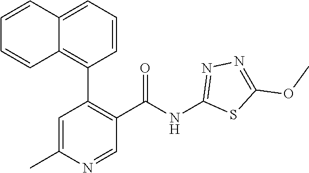 [SMILES: COc1nnc(NC(=O)c2cnc(C)cc2-c2cccc3ccccc23)s1] |
| 399 | 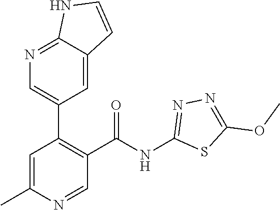 |
| 400 | 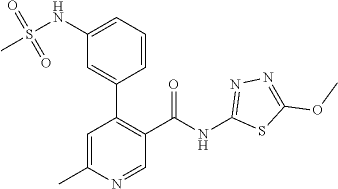 |
| 401 | 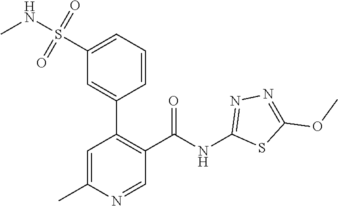 |
| 402 | 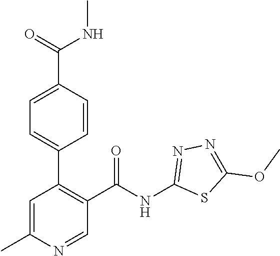 |
| 403 | 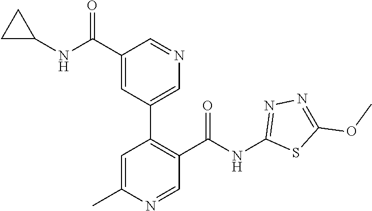 [SMILES: COc1nnc(NC(=O)c2cnc(C)cc2-c2cncc(C(=O)NC3CC3)c2)s1] |
| 404 | 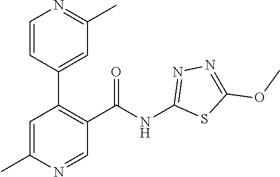 |
| 405 | 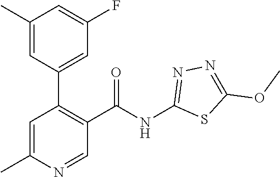 |
| 406 | 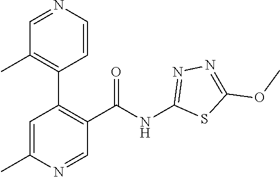 |
| 407 | 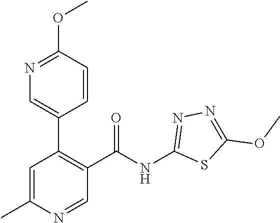 |
| 408 | 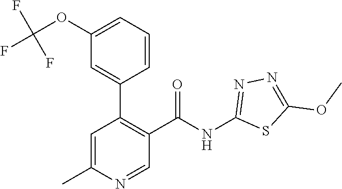 [SMILES: COc1nnc(NC(=O)c2cnc(C)cc2-c2cccc(OC(F)(F)F)c2)s1] |
| 409 | 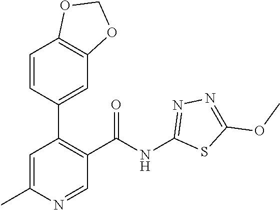 |
| 410 | 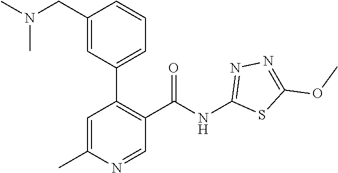 |
| 411 | 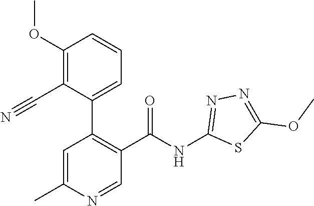 [SMILES: COc1nnc(NC(=O)c2cnc(C)cc2-c2cccc(OC)c2C#N)s1] |
| 412 | 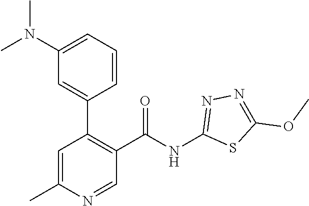 |
| 413 | 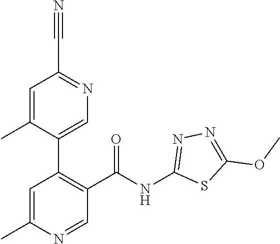 |
| 414 | 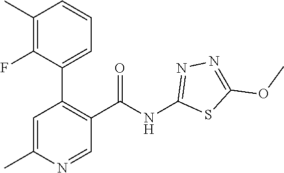 |
| 415 | 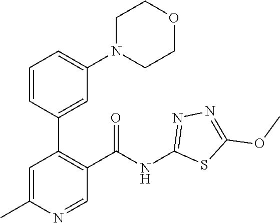 |
| 416 | 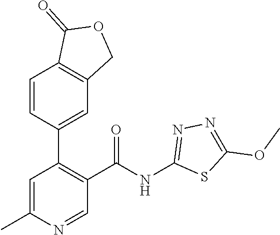 |
| 417 | 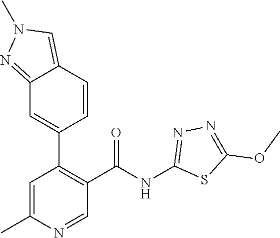 |
| 418 | 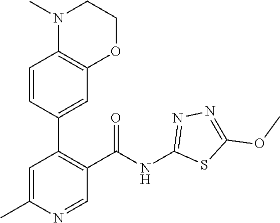 |
| 419 | 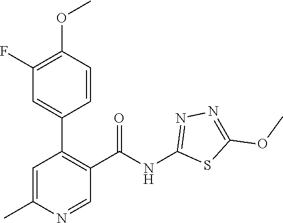 |
| 420 | 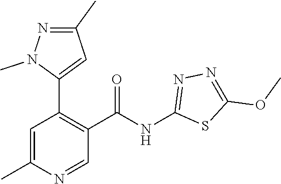 |
| 421 | 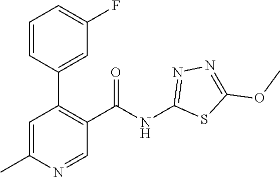 |
| 422 | 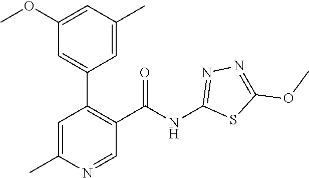 |
| 423 | 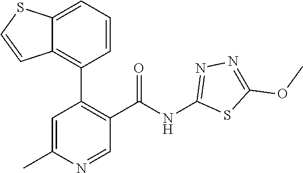 [SMILES: COc1nnc(NC(=O)c2cnc(C)cc2-c2cccc3sccc23)s1] |
| 424 | 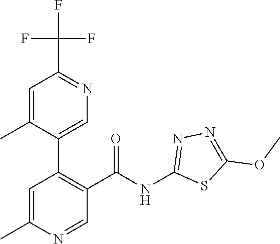 |
| 425 | 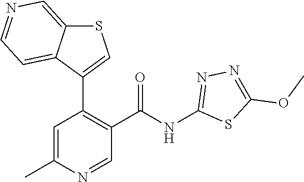 [SMILES: COc1nnc(NC(=O)c2cnc(C)cc2-c2csc3cnccc23)s1] |
| 426 | 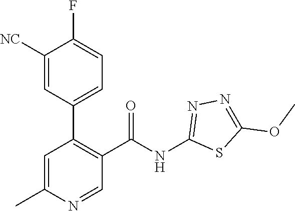 |
| 427 | 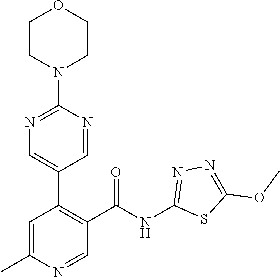 |
| 428 | 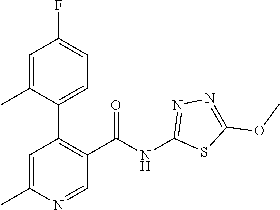 |
| 429 | 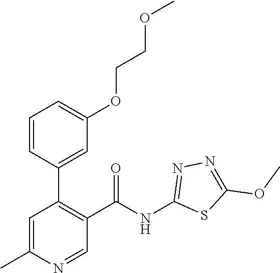 |
| 430 | 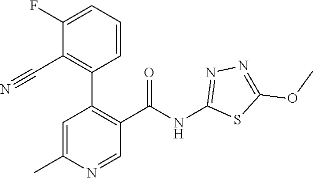 [SMILES: COc1nnc(NC(=O)c2cnc(C)cc2-c2cccc(F)c2C#N)s1] |
| 431 | 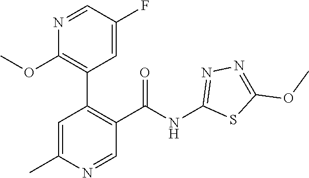 |
| 432 | 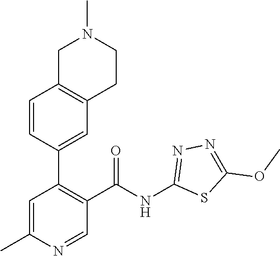 |
| 433 | 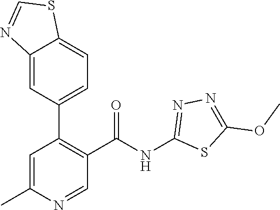 |
| 434 | 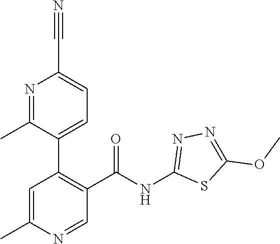 |
| 435 | 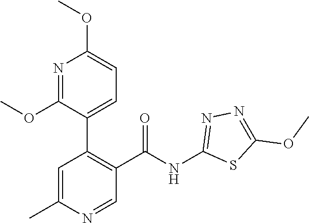 |
| 436 | 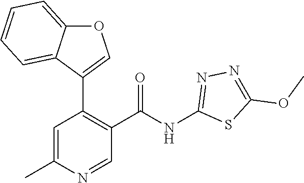 [SMILES: COc1nnc(NC(=O)c2cnc(C)cc2-c2coc3ccccc23)s1] |
| 437 | 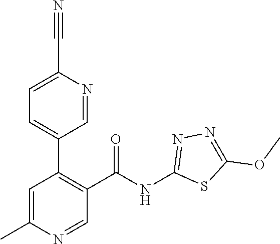 |
| 438 | 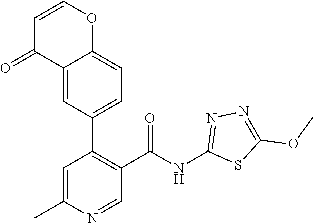 [SMILES: COc1nnc(NC(=O)c2cnc(C)cc2-c2ccc3occc(=O)c3c2)s1] |
| 439 | 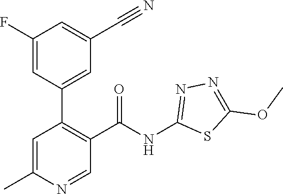 |
| 440 | 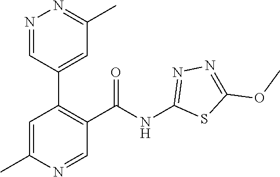 |
| 441 | 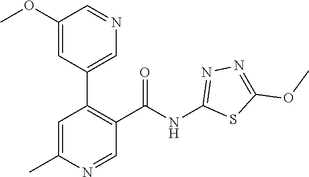 |
| 442 | 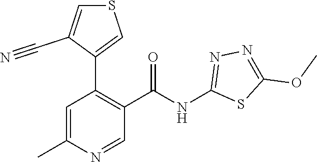 [SMILES: COc1nnc(NC(=O)c2cnc(C)cc2-c2cscc2C#N)s1] |
| 443 | 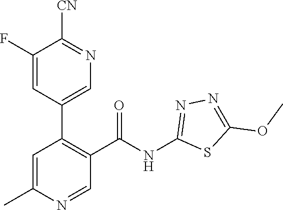 |
| 444 | 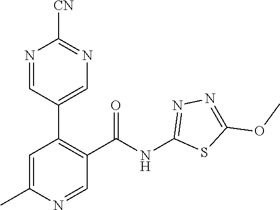 |
| 445 | 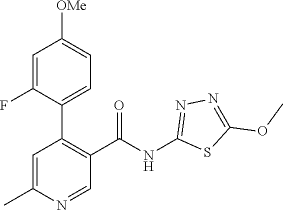 |
| 446 | 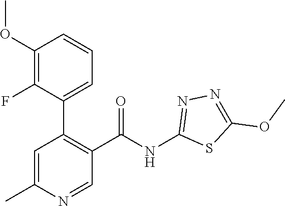 |
| 447 | 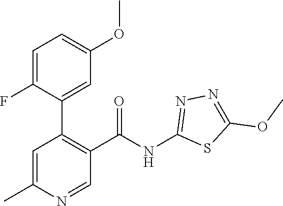 |
| 448 | 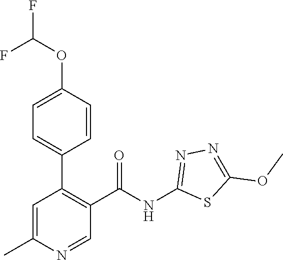 |
| 449 | 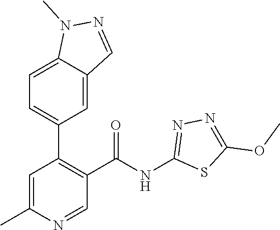 |
| 450 | 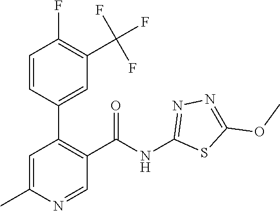 |
| 451 | 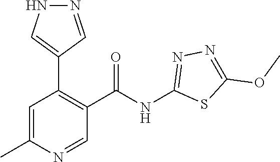 |
| 452 | 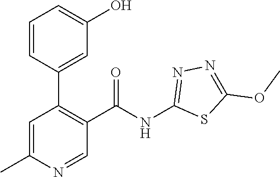 |
| 453 | 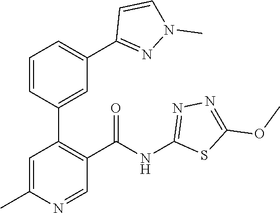 |
| 454 | 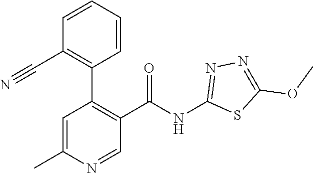 [SMILES: COc1nnc(NC(=O)c2cnc(C)cc2-c2ccccc2C#N)s1] |
| 455 | 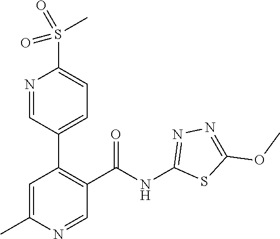 |
| 456 | 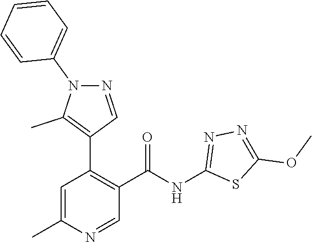 [SMILES: COc1nnc(NC(=O)c2cnc(C)cc2-c2cnn(-c3ccccc3)c2C)s1] |
| 457 | 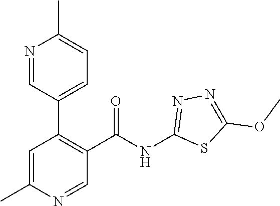 |
| 458 | 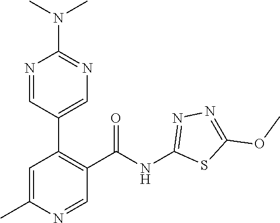 |
| 459 | 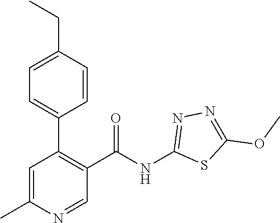 |
| 460 | 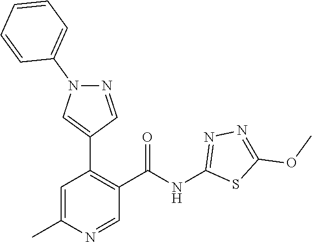 [SMILES: COc1nnc(NC(=O)c2cnc(C)cc2-c2cnn(-c3ccccc3)c2)s1] |
| 461 | 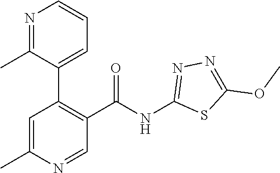 |
| 462 | 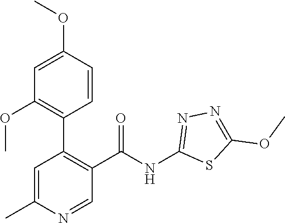 |
| 463 | 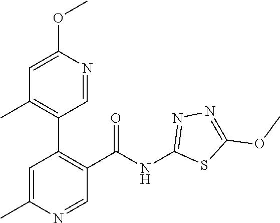 |
| 464 | 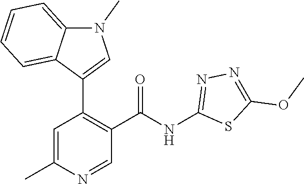 [SMILES: COc1nnc(NC(=O)c2cnc(C)cc2-c2cn(C)c3ccccc23)s1] |
| 465 | 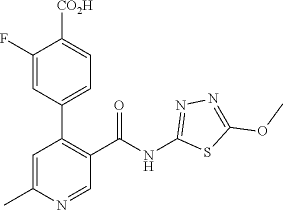 |
| 466 | 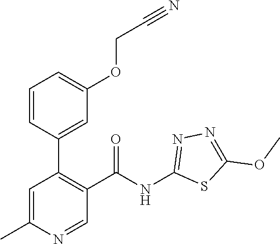 |
| 467 | 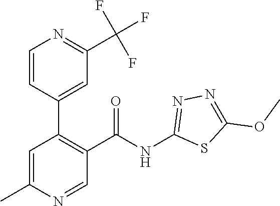 |
| 468 | 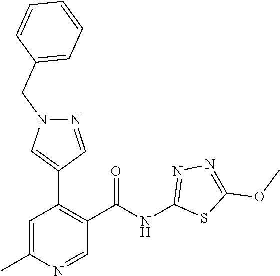 |
| 469 | 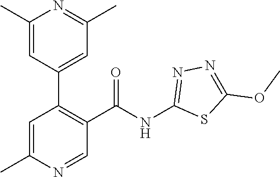 |
| 470 | 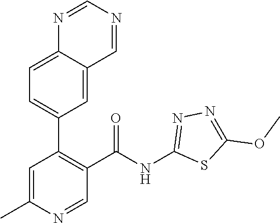 |
| 471 | 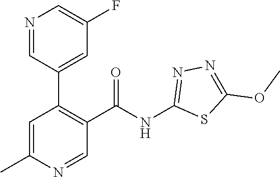 |
| 472 | 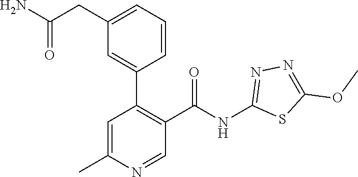 [SMILES: COc1nnc(NC(=O)c2cnc(C)cc2-c2cccc(CC(N)=O)c2)s1] |
| 473 | 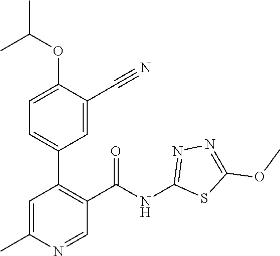 |
| 474 | 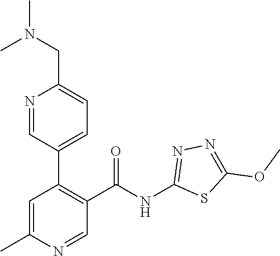 |
| 475 | 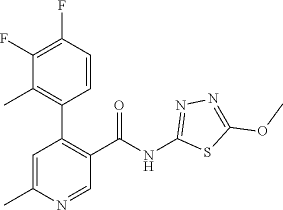 |
| 476 | 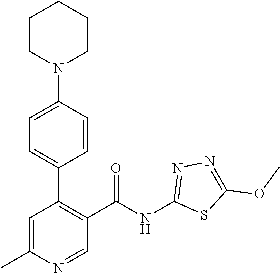 |
| 477 | 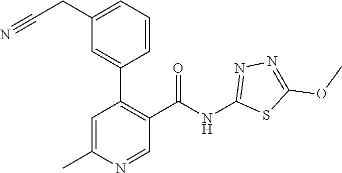 [SMILES: COc1nnc(NC(=O)c2cnc(C)cc2-c2cccc(CC#N)c2)s1] |
| 478 | 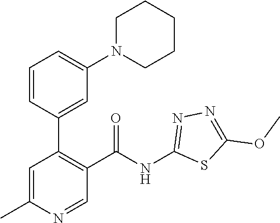 |
| 479 | 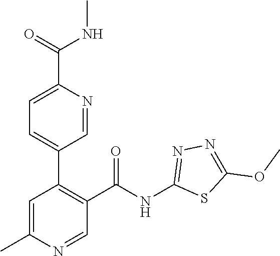 |
| 480 | 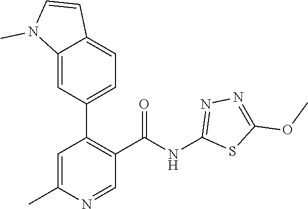 |
| 481 | 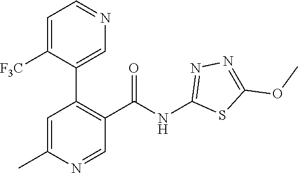 |
| 482 | 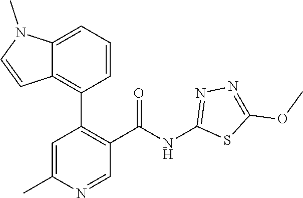 [SMILES: COc1nnc(NC(=O)c2cnc(C)cc2-c2cccc3c2ccn3C)s1] |
| 483 | 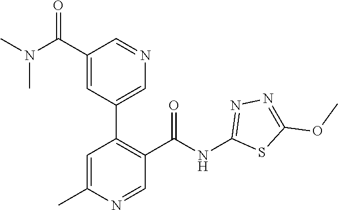 |
| 484 | 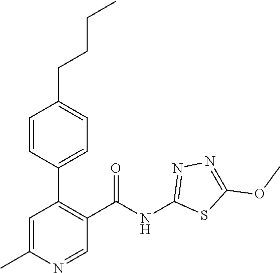 |
| 485 | 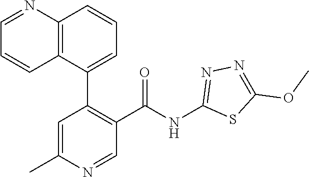 [SMILES: COc1nnc(NC(=O)c2cnc(C)cc2-c2cccc3ncccc23)s1] |
| 486 | 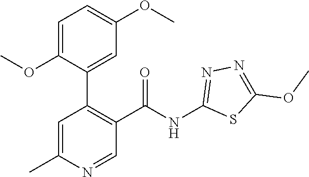 |
| 487 | 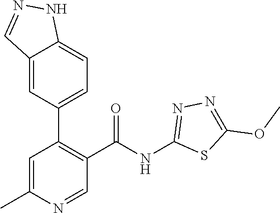 |
| 488 | 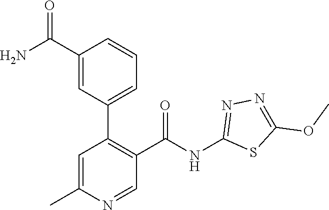 [SMILES: COc1nnc(NC(=O)c2cnc(C)cc2-c2cccc(C(N)=O)c2)s1] |
| 489 | 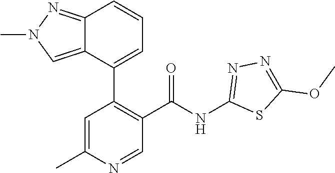 |
| 490 | 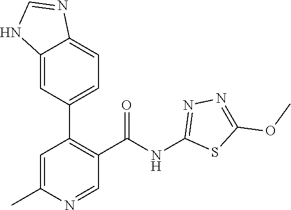 |

In some embodiments, the compounds or pharmaceutically acceptable salts thereof is a compound from Table 1, Table 2 or the Examples.

In some embodiments, the compounds or pharmaceutically acceptable salts thereof is a compound from Table 1, Table 2, Table 3, or the Examples.

In some embodiments, the compounds or pharmaceutically acceptable salts thereof is a compound from Table 1, Table 2, or Table 3.

In some embodiments, the compounds or pharmaceutically acceptable salts thereof is a compound from Table 1 or Table 2.

In some embodiments, the compounds or pharmaceutically acceptable salts thereof is a compound from Table 1.

In some embodiments, the compounds or pharmaceutically acceptable salts thereof is a compound from Table 2.

In some embodiments, the compounds or pharmaceutically acceptable salts thereof is a compound from Table 3.

In some embodiments, the compounds or pharmaceutically acceptable salts thereof is a compound selected from examples 86, 87, 89, 90, 96, 97, 106, 108, 111, and 113.

In some embodiments, the compounds or pharmaceutically acceptable salts thereof is a compound selected from examples 79 to 85, 88, 91 to 93, 100 to 105, 109, 112, 114 to 119, 122, 124 to 143, and 145 to 233.

The compounds disclosed herein can be prepared as described in the examples section, below. For those compounds without detailed synthetic write-ups, it is understood that these compounds can be prepared following the general procedures described herein.

### Assay

The ability of compounds of the disclosure to inhibit Polθ can be measured as described in the biological assay below.

### Pharmaceutical Composition

The compounds of Formula (I) or a subembodiment or a pharmaceutically acceptable salt thereof provided herein may be in the form of compositions suitable for administration to a subject. In general, such compositions are pharmaceutical compositions comprising a compound of Formula (I) or a subembodiment or a pharmaceutically acceptable salt thereof and one or more pharmaceutically acceptable excipients. In certain embodiments, the compound of Formula (I) or a subembodiment or a pharmaceutically acceptable salt thereof is present in a therapeutically effective amount. The pharmaceutical compositions may be used in all the methods disclosed herein; thus, for example, the pharmaceutical compositions can be administered ex vivo or in vivo to a subject in order to practice the therapeutic methods and uses described herein.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat the diseases, disorders and conditions contemplated by the present disclosure.

The pharmaceutical compositions containing the active ingredient (e.g., a compound of Formula (I) or a subembodiment or a pharmaceutically acceptable salt thereof) may be in a form suitable for oral use, for example, as tablets, capsules, troches, lozenges, aqueous or oily suspensions, dispersible powders or granules, emulsions, hard or soft capsules, or syrups, solutions, microbeads or elixirs.

Tablets, capsules and the like contain the active ingredient in admixture with non-toxic pharmaceutically acceptable excipients which are suitable for the manufacture of tablets, capsules, and the like. These excipients can include diluents, granulating agents, disintegrating agents, binding agents, and lubricating agents.

The tablets, capsules and the like suitable for oral administration may be uncoated or coated by known techniques to delay disintegration and absorption in the gastrointestinal tract and thereby provide a sustained action.

Formulations for oral use may also be presented as hard gelatin capsules wherein the active ingredient is mixed with an inert solid diluent, or as soft gelatin capsules wherein the active ingredient is mixed with water or an oil medium.

Aqueous suspensions contain the active materials in admixture with excipients suitable for the manufacture thereof. Such excipients can include suspending agents, dispersing agents and wetting agents. The aqueous suspensions may also contain one or more preservatives.

Oily suspensions may be formulated by suspending the active ingredient in an oil. Suitable oils are known in the art. The oily suspensions may contain additional agents such as a thickening agent or a sweetening agent.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions may also be in the form of oil-in-water emulsions. Suitable emulsifying agents are known in the art.

The pharmaceutical compositions typically comprise a therapeutically effective amount of a compound of Formula (I) or a subembodiment or a pharmaceutically acceptable salt thereof, and one or more pharmaceutically acceptable excipient. Suitable pharmaceutically acceptable excipients include, but are not limited to, antioxidants, preservatives, emulsifying agents, suspending agents, dispersing agents, solvents, fillers, bulking agents, detergents, buffers, vehicles, diluents, and/or adjuvants. Those skilled in the art will readily recognize a variety of excipients that can be used in the pharmaceutical compositions and dosage forms contemplated herein.

Depot injections, which are generally administered subcutaneously or intramuscularly, may also be utilized to release the compound of Formula (I) or a subembodiment or a pharmaceutically acceptable salt thereof disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. The suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, which are known in the art.

A compound of Formula (I) or a subembodiment or a pharmaceutically acceptable salt thereof may also be administered in the form of suppositories for rectal administration or sprays for nasal or inhalation use. The suppositories can be prepared by mixing the drug with a suitable non-irritating excipient which is solid at ordinary temperatures but liquid at the rectal temperature and will therefore melt in the rectum to release the drug. Such materials are known in the art.

All the compounds and pharmaceutical compositions provided herein can be used in all the methods provided herein. For example, the compounds and pharmaceutical compositions provided herein can be used in all the methods for treatment and/or prevention of all diseases or disorders provided herein. Thus, the compounds and pharmaceutical compositions provided herein are for use as a medicament.

### Routes of Administration

Compounds of Formula (I) or a subembodiment or a pharmaceutically acceptable salt thereof and compositions containing the same may be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to administer the compounds of Formula (I) or a subembodiment or a pharmaceutically acceptable salt thereof over a defined period of time. Particular embodiments of the present invention contemplate oral administration.

### Combination Therapy

The present invention contemplates the use of compounds of Formula (I) or a subembodiment or a pharmaceutically acceptable salt thereof in combination with one or more active therapeutic agents (e.g., chemotherapeutic agents) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the compounds of Formula (I) or a subembodiment or a pharmaceutically acceptable salt thereof are administered or applied sequentially, e.g., where one agent is administered prior to one or more other agents. In other embodiments, the compounds of Formula (I) or a subembodiment or a pharmaceutically acceptable salt thereof are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The compounds of Formula (I) or a subembodiment or a pharmaceutically acceptable salt thereof may be used in combination with at least one other (active) agent in any manner appropriate under the circumstances. In one embodiment, treatment with the at least one active agent and at least one compound of Formula (I) or a subembodiment or a pharmaceutically acceptable salt thereof is maintained over a period of time. In another embodiment, treatment with the at least one active agent is reduced or discontinued (e.g., when the subject is stable), while treatment with the compound of Formula (I) or a subembodiment or a pharmaceutically acceptable salt thereof is maintained at a constant dosing regimen. In a further embodiment, treatment with the at least one active agent is reduced or discontinued (e.g., when the subject is stable), while treatment with a compound of Formula (I) or a subembodiment or a pharmaceutically acceptable salt thereof is reduced (e.g., lower dose, less frequent dosing or shorter treatment regimen). In yet another embodiment, treatment with the at least one active agent is reduced or discontinued (e.g., when the subject is stable), and treatment with the compound of Formula (I) or a subembodiment or a pharmaceutically acceptable salt thereof is increased (e.g., higher dose, more frequent dosing or longer treatment regimen). In yet another embodiment, treatment with the at least one active agent is maintained and treatment with the compound of Formula (I) or a subembodiment or a pharmaceutically acceptable salt thereof is reduced or discontinued (e.g., lower dose, less frequent dosing or shorter treatment regimen). In yet another embodiment, treatment with the at least one active agent and treatment with the compound of Formula (I) or a subembodiment or a pharmaceutically acceptable salt thereof are reduced or discontinued (e.g., lower dose, less frequent dosing or shorter treatment regimen).

The present disclosure provides methods for treating cancer with a compound of Formula (I) or a subembodiment or a pharmaceutically acceptable salt thereof and at least one additional therapeutic or diagnostic agent.

In some embodiments, the compound of Formula (I) or a subembodiment or a pharmaceutically acceptable salt thereof is administered in combination with at least one additional therapeutic agent. In some embodiments, the additional therapeutic agent is a signal transduction inhibitor (STI) or chemotherapeutic agent.

In certain embodiments, the present disclosure provides methods for treating cancer comprising administration of a compound of Formula (I) or a subembodiment or a pharmaceutically acceptable salt thereof described herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Agents involved in immunomodulation can also be used in combination with one or more compounds of Formula (I) or a subembodiment or a pharmaceutically acceptable salt thereof described herein for the suppression of tumor growth in cancer patients.

In certain embodiments, the present disclosure provides methods for treating cancer comprising administration of a compound of Formula (I) or a subembodiment or a pharmaceutically acceptable salt thereof described herein in combination with a chemotherapeutic agents.

Chemotherapeutic agents also include anti-hormonal agents that act to regulate or inhibit hormonal action on tumors such as anti-estrogens. In certain embodiments, combination therapy comprises administration of a hormone or related hormonal agent.

The present disclosure also contemplates the use of the compounds of Formula (I) or a subembodiment or a pharmaceutically acceptable salt thereof described herein in combination with immune checkpoint inhibitors. The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism. Examples of immune checkpoint inhibitors include but are not limited to CTLA-4, PD-1, PD-L1, BTLA, TIM3, LAG3, OX40, 41BB, VISTA, CD96, TGFβ, CD73, CD39, A2AR, A2BR, IDO1, TDO2, Arginase, B7-H3, B7-H4. Cell-based modulators of anti-cancer immunity are also contemplated. Examples of such modulators include but are not limited to chimeric antigen receptor T-cells, tumor infiltrating T-cells and dendritic-cells.

The present disclosure encompasses pharmaceutically acceptable salts, acids or derivatives of any of the above.

### Dosing

The compounds of Formula (I) or a subembodiment or a pharmaceutically acceptable salt thereof provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of a compound of Formula (I) or a subembodiment or a pharmaceutically acceptable salt thereof, as provided herein, may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, the compounds of Formula (I) or a subembodiment or a pharmaceutically acceptable salt thereof disclosed herein may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the compound of Formula (I) or a subembodiment or a pharmaceutically acceptable salt thereof is contained in a “unit dosage form”. The phrase “unit dosage form” refers to physically discrete units, each unit containing a predetermined amount of the compound of Formula (I) or a subembodiment or a pharmaceutically acceptable salt thereof, either alone or in combination with one or more additional agents, sufficient to produce the desired effect. It will be appreciated that the parameters of a unit dosage form will depend on the particular agent and the effect to be achieved.

### Kits

The present invention also contemplates kits comprising a compound of Formula (I) or a subembodiment or a pharmaceutically acceptable salt thereof. The kits are generally in the form of a physical structure housing various components, as described below, and may be utilized, for example, in practicing the methods described above.

A kit can include one or more of the compound of Formula (I) or a subembodiment or a pharmaceutically acceptable salt thereof disclosed herein (provided in, e.g., a sterile container), which may be in the form of a pharmaceutical composition suitable for administration to a subject. The compound of Formula (I) or a subembodiment or a pharmaceutically acceptable salt thereof can be provided in a form that is ready for use (e.g., a tablet or capsule) or in a form requiring, for example, reconstitution or dilution (e.g., a powder) prior to administration. When the compounds of Formula (I) or a subembodiment or a pharmaceutically acceptable salt thereof are in a form that needs to be reconstituted or diluted by a user, the kit may also include diluents (e.g., sterile water), buffers, pharmaceutically acceptable excipients, and the like, packaged with or separately from the compounds of Formula (I) or a subembodiment or a pharmaceutically acceptable salt thereof. When combination therapy is contemplated, the kit may contain the several agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXAMPLES

The following examples and references (intermediates) are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (C), and pressure is at or near atmospheric. Standard abbreviations are used, including the following: THF=tetrahydrofuran; DIEA=diisopropylethylamine; EtOAc=ethyl acetate; NMP=N-methylpyridine, TFA=trifluoroacetic acid; DCM=dichloromethane; Cs₂CO₃=cesium carbonate; XPhos Pd G3=2-dicyclohexylphosphino-2′,4′,6′-triisopropyl-1, l′-biphenyl)(2-(2′-amino-1,1′-biphenyl))palladium-(II) methanesulfonate; LiCl=lithium chloride; POCl₃=phosphoryl chloride; PE=petroleum ether; DMSO=dimethylsulfoxide; HCl=hydrochloric acid; Na₂SO₄=sodium sulfate; DMF=dimethylformamide; NaOH=sodium hydroxide; K₂CO₃=potassium carbonate; MeCN=acetonitrile; BOC=tert-butoxycarbonyl; MTBE=methyl tert-butyl ether; MeOH=methanol; NaHCO₃=sodium bicarbonate; NaBH₃CN=sodium cyanoborohydride; EtOH=ethanol; PCl₅=phosphorus pentachloride; NH₄OAc=ammonium acetate; Et₂O=ether; HOAc=acetic acid; Ac₂O=acetic anhydride; i-PrOH=isopropanol; NCS=N-chlorosuccinimide; K₃PO₄=potassium phosphate; Pd(dtbpf)Cl₂=1,1′-Bis(di-tert-butylphosphino)ferrocene)dichloro-palladium(II); Zn(CN)₂=Zinc cyanide; Pd(PPh₃)₄=tetrakis(triphenylphosphine)palladium(0); Et₃N=triethylamine; CuCN=copper cyanide; t-BuONO=tert-butyl nitrite; HATU=1-(bis(dimethylamino)methylene)-1H-1,2,3-triazolo(4,5-b)pyridinium 3-oxide hexafluorophosphate; DBU=1,8-diazabicyclo(5.4.0)undec-7-ene; LiAlH₄=lithium aluminium hydride; NH₃=ammonia; H₂SO₄=sulfuric acid; H₂O₂=hydrogen peroxide; EDCI=N-(3-dimethylaminopropyl)-N′-ethylcarbodiimide hydrochloride; HOBT=1-hydroxy benzotriazole hydrate; DHP=dihydropyran; TsOH=p-Toluenesulfonic acid; FA=formic acid; TCFH=N,N,N,N′-tetramethylchloroformamidinium hexafluorophosphate; NMI=N-methylimidazole; Pd(dppf)Cl₂=(1,1′-Bis(diphenylphosphino)ferrocene)dichloropalladium(II); Pd(dppf)Cl₂-DCM=(1,1′-Bis(diphenylphosphino)ferrocene)dichloropalladium(II), complex with dichloromethane.

When the absolute or relative stereochemistry of one or more examples is not determined, the text of the Example indicates the same. The reported characterizations in these examples and the tables of compounds arbitrarily assign absolute stereochemistry. It is understood that routine work well within the skill of the ordinary artisan can confirm the absolute stereochemistry for each relevant example disclosed herein.

### Synthetic Examples

### Intermediate A

### 3-(2-methoxyphenyl) pyridine-4-carboxylic Acid

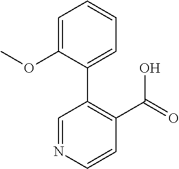 [SMILES: COc1ccccc1-c1cnccc1C(=O)O]

To a solution of 3-bromopyridine-4-carboxylic acid (2.0 g, 9.9 mmol) in dioxane (10 mL) and water (10 mL) was added 2-methoxyphenylboronic acid (2.3 g, 14.9 mmol), Na₂CO₃ (1.1 g, 9.9 mmol) and Pd(PPh₃)₄ (1.1 g, 0.99 mmol) at room temperature under nitrogen. The mixture was stirred at 100° C. overnight. The mixture was cooled to room temperature and diluted with water. The mixture was extracted with EtOAc (2×). The aqueous layer was acidified to pH 6 with HCl (1 M). A solid formed and mixture was filtered to afford 3-(2-methoxyphenyl) pyridine-4-carboxylic acid as a white solid, which was used to next step without further purification.

### Intermediate B

### 5-((5-chloropyridin-2-yl)methoxy)-1,3,4-thiadiazol-2-amine

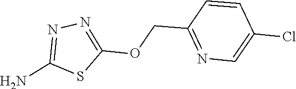 [SMILES: Nc1nnc(OCc2ccc(Cl)cn2)s1]

### Step 1. Preparation of (5-chloropyridin-2-yl)methanol

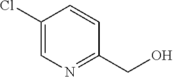

To a solution of methyl 5-chloropicolinate (95 g, 554 mmol) in MeOH (950 mL) was added NaBH₄ (42.0 g, 1.11 mol) in portions at 0° C. Then the mixture was stirred at rt for 2 h. The mixture was poured into H₂O. Mixture was cooled to 0° C. and 6 N HCl was added until pH of solution was 1˜2. The temperature of the solution was 0-10° C. Then the mixture was concentrated under reduce pressure to remove MeOH. 6 N NaOH was added until the pH of the solution was 8˜10. The mixture was extracted with EtOAc (3×). The combined organic layers were dried over Na₂SO₄, filtered and concentrated under reduce pressure to afford the title compound (158 g) as a yellow oil, which was used in the next step without further purification.

### Step 2. Preparation of 5-((5-chloropyridin-2-yl)methoxy)-1,3,4-thiadiazol-2-amine

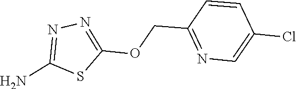 [SMILES: Nc1nnc(OCc2ccc(Cl)cn2)s1]

To a solution of NaH (65.7 g, 1.64 mol, 60.0% purity) in THF (1.20 L) was added a solution of (5-chloropyridin-2-yl)methanol (158 g, 1.10 mol) in THF (400 mL) at 5° C. dropwise. The mixture was stirred at 5° C. for 1 h. Then 2-amino-5-bromo-1,3,4-thiadiazole (237 g, 1.31 mol) was added in portions at 5° C. The mixture was stirred at 5° C. for 4 h. The mixture was poured into H₂O and extracted with EtOAc (4×). The combined organic layer was dried over Na₂SO₄, filtered and concentrated under reduce pressure. The residue was diluted with MeOH and slurry was stirred at 25° C. for 0.5 h. The solids were collected and diluted with MeOH. The slurry was stirred at 80° C. for 2 h. The solids were collected to afford 5-((5-chloropyridin-2-yl)methoxy)-1,3,4-thiadiazol-2-amine (57.6 g, 21% yield) as a grey solid.

### Intermediate C

### 4-(2-methoxyphenyl)-6-methylnicotinic Acid

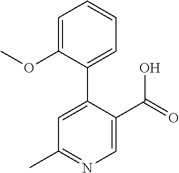 [SMILES: COc1ccccc1-c1cc(C)ncc1C(=O)O]

### Step 1 methyl 4-chloro-6-methylnicotinate

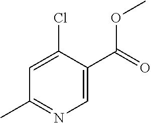

To a stirred solution of 4-chloro-6-methylnicotinic acid (5.0 g, 29.1 mmol) in dichloromethane (100 mL) was added methanol (10 mL) and (diazomethyl)trimethylsilane (29 mL, 58.3 mmol) dropwise at 0° C. The resulting solution was stirred at room temperature for 16 hr. The organic solvent was removed under vacuum. The resulting residue was dissolved in dichloromethane (5 mL) and purified by Combi Flash (Biotage Isolera Prime) which applied to a 80.0 g silica gel column that was eluted with 0˜50% ethyl acetate in petroleum ether within 30 min to afford methyl 4-chloro-6-methylnicotinate (4.5 g, 81% yield) as a yellow oil. MS (ESI) calc'd for (C₈H₈ClNO₂) (M+1)⁺, 186.0. found 186.0.

### Step 2 methyl 4-(2-methoxyphenyl)-6-methylnicotinate

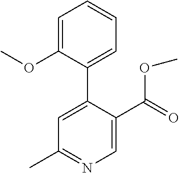

To a stirred solution of methyl 4-chloro-6-methylnicotinate (500.0 mg, 2.69 mmol) and 2-methoxyphenylboronic acid (819.0 mg, 5.39 mmol) in 1,4-Dioxane (1 mL) were sequentially added water (0.2 mL), (1,1′-Bis(diphenylphosphino)ferrocene)dichloropalladium(II) (591.0 mg, 0.81 mmol) and potassium carbonate (1.1 g, 8.08 mmol) at 23° C. The resulting solution was stirred at 80° C. for 2 hr under nitrogen. The combined organic layers was diluted with ethyl acetate and the suspension was filtered. The filtrate was collected and concentrated under vacuum. The resulting residue was dissolved in dichloromethane (5 mL) and purified by Combi Flash (Biotage Isolera Prime) which applied to a 40 g silica gel column that was eluted with 0˜35% ethyl acetate in petroleum ether within 30 min to afford methyl 4-(2-methoxyphenyl)-6-methylnicotinate (700.0 mg, 98%) as a yellow oil. MS (ESI) calc'd for (C₁₅H₁₅NO3) (M+1)⁺, 258.1. found 258.0.

### Step 3 4-(2-methoxyphenyl)-6-methylnicotinic Acid

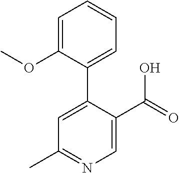 [SMILES: COc1ccccc1-c1cc(C)ncc1C(=O)O]

To a stirred solution of methyl 4-(2-methoxyphenyl)-6-methylnicotinate (400.0 mg, 1.56 mmol) in methanol (3 mL) were added water (3 mL) and sodium hydroxide (249.0 mg, 6.22 mmol) at 23° C. The resulting solution was stirred at 23° C. for 2 hr. The organic solvent was removed under vacuum. The aqueous layer was acidified with citric acid solution to pH ˜6 and extracted with ethyl acetate. The combined organic layers were washed with brine, dried over anhydrous sodium sulfate, filtered, and concentrated under vacuum to afford 4-(2-methoxyphenyl)-6-methylnicotinic acid (342.0 mg, crude) as a yellow oil. MS (ESI) calc'd for (C₁₄H₁₃NO₃) (M+1)⁺, 244.1. found 244.0.

### Intermediate D

### 4-(2-(difluoromethoxy)-6-fluorophenyl)-6-methylnicotinic Acid

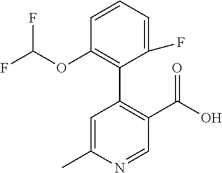 [SMILES: Cc1cc(-c2c(F)cccc2OC(F)F)c(C(=O)O)cn1]

### Step-1: 2-bromo-1-(difluoromethoxy)-3-fluorobenzene

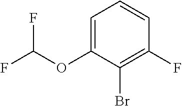 [SMILES: Fc1cccc(OC(F)F)c1Br]

To a stirred solution of 2-bromo-3-fluorophenol (25.00 g, 130.89 mmol) in DMF (100.00 mL) and H₂O (10 mL) were added sodium 2-chloro-2,2-difluoroacetate (16.95 g, 130.89 mmol) in portions at room temperature. The resulting mixture was stirred at 80° C. for 16 h. After cooled down to room temperature, the resulting mixture was diluted with water extracted with ethyl acetate. The combined organic layers were washed with brine, dried over anhydrous sodium sulfate, filtered, and concentrated under vacuum. The residue was purified by flash column chromatography with 0˜10% ethyl acetate in petroleum ether to afford 2-bromo-1-(difluoromethoxy)-3-fluorobenzene (12.2 g, 34.8%) as a white oil. MS (ESI) calc'd for (C₇H₄BrF₃O) (M+1)⁺, 240.9. found 241.0.

### Step-2: 2-(2-(difluoromethoxy)-6-fluorophenyl)-4,4,5,5-tetramethyl-1,3,2-dioxaborolane

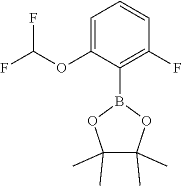 [SMILES: CC1(C)OB(c2c(F)cccc2OC(F)F)OC1(C)C]

To a degassed solution of 2-bromo-1-(difluoromethoxy)-3-fluorobenzene (3.00 g, 12.44 mmol) in dioxane (15.00 mL) were added Pd(dppf)Cl₂ (0.91 g, 1.245 mmol), B₂Pin₂ (6.30 g, 24.89 mmol) and KOAc (2.44 g, 24.89 mmol). The resulting mixture was stirred at 90° C. overnight under nitrogen atmosphere. The solvent was removed under vacuum, the residue was purified by flash column chromatography with 0˜10% ethyl acetate in petroleum ether to afford 2-(2-(difluoromethoxy)-6-fluorophenyl)-4,4,5,5-tetramethyl-1,3,2-dioxaborolane (3.3 g, 46.01%) as a green solid.

### Step-3: methyl 4-(2-(difluoromethoxy)-6-fluorophenyl)-6-methylpyridine-3-carboxylate

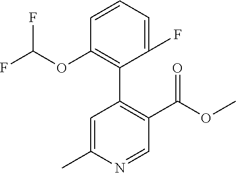 [SMILES: COC(=O)c1cnc(C)cc1-c1c(F)cccc1OC(F)F]

To a degassed solution of 2-(2-(difluoromethoxy)-6-fluorophenyl)-4,4,5,5-tetramethyl-1,3,2-dioxaborolane (3.00 g, 10.41 mmol) in dioxane (15.00 mL) and water (3 mL) were added methyl 4-chloro-6-methylpyridine-3-carboxylate (1.93 g, 10.39 mmol), K₂CO₃ (4.32 g, 31.25 mmol) and Pd(dppf)Cl₂ (762.00 mg, 1.041 mmol). The resulting mixture was stirred at 80° C. for 12 h under nitrogen atmosphere. The reaction mixture was quenched with water and extracted with ethyl acetate. The combined organic layers were washed with brine, dried over anhydrous sodium sulfate, filtered, and concentrated under vacuum. The residue was purified by flash column chromatography with 0˜50% ethyl acetate in petroleum ether to afford methyl 4-(2-(difluoromethoxy)-6-fluorophenyl)-6-methylpyridine-3-carboxylate (700 mg, 19%) as a yellow solid. MS (ESI) calc'd for (C₁₅H₁₂F₃NO₃) (M+1)⁺, 312.1. found 312.0.

### Step-4: 4-(2-(difluoromethoxy)-6-fluorophenyl)-6-methylpyridine-3-carboxylic Acid

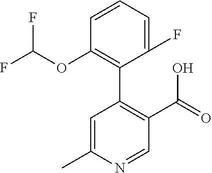 [SMILES: Cc1cc(-c2c(F)cccc2OC(F)F)c(C(=O)O)cn1]

To a stirred solution of methyl 4-(2-(difluoromethoxy)-6-fluorophenyl)-6-methylpyridine-3-carboxylate (470.00 mg, 1.51 mmol) in THF (3.00 mL) and water (3 mL) was added LiOH·H₂O (253.68 mg, 6.040 mmol). The resulting mixture was stirred at room temperature for 16 h. The organic solvent was removed under vacuum and then diluted with water. The solution was acidified by HCl (1 N) to pH ˜6. The resulting mixture was extracted with ethyl acetate. The combined organic layers were washed with brine, dried over anhydrous sodium sulfate, filtered, and concentrated under vacuum to afford 4-(2-(difluoromethoxy)-6-fluorophenyl)-6-methylpyridine-3-carboxylic acid (370 mg, 82.4%) as a white solid. MS (ESI) calc'd for (C₁₄H₁₀F₃NO₃) (M+1)⁺, 298.1. found 298.0.

### Intermediate E

### 4-(2-fluoro-6-methoxyphenyl)-6-methylnicotinic Acid

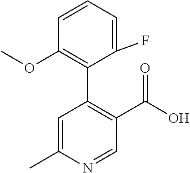 [SMILES: COc1cccc(F)c1-c1cc(C)ncc1C(=O)O]

The title compound was prepared following the procedures for Intermediate C, Step 2 and 3 employing methyl 3-bromoisonicotinate and 2-fluoro-6-methoxyphenylboronic acid to afford 4-(2-fluoro-6-methoxyphenyl)-6-methylnicotinic acid as a white solid which was used without further purification.

### Intermediate F

### 5′-methoxy-2′,6-dimethyl-(4,4′-bipyridine)-3-carboxylic Acid

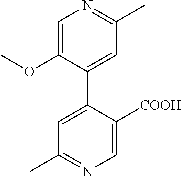 [SMILES: COc1cnc(C)cc1-c1cc(C)ncc1C(=O)O]

### Step 1 benzyl 4-chloro-6-methylnicotinate

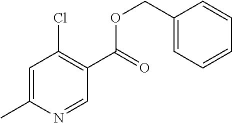 [SMILES: Cc1cc(Cl)c(C(=O)OCc2ccccc2)cn1]

A mixture of 4-chloro-6-methylpyridine-3-carboxylic acid (10.00 g, 58.3 mmol) and Cs₂CO₃ (37.98 g, 116.6 mmol) in DMF (100 mL) was added benzyl bromide (14.95 g, 87.45 mmol). The resulting mixture was stirred at room temperature for 16 h. The reaction mixture was quenched with water and extracted with ethyl acetate. The combined organic layers were washed with brine, dried over anhydrous sodium sulfate, filtered, and concentrated under vacuum. The residue was purified by flash chromatography on silica gel with 0˜30% ethyl acetate in petroleum ether to afford benzyl 4-chloro-6-methylnicotinate (12.94 g, 84.8%) as a yellow oil. MS (ESI) calc'd for (C₁₄H₁₂ClNO₂) (M+1)⁺, 262.0. found 262.1.

### Step 2 benzyl 2′-chloro-5′-methoxy-6-methyl-(4,4′-bipyridine)-3-carboxylate

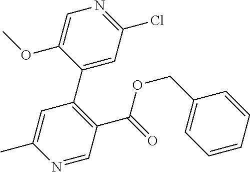 [SMILES: COc1cnc(Cl)cc1-c1cc(C)ncc1C(=O)OCc1ccccc1]

To a degassed mixture of methyl benzyl 4-chloro-6-methylpyridine-3-carboxylate (6.00 g, 22.926 mmol) and 2-chloro-5-methoxypyridin-4-ylboronic acid (4.30 g, 22.926 mmol) in 1,4-dioxane (50 mL) and H₂O (5 mL) were added K₂CO₃ (9.51 g, 0.069 mmol) and Pd(DtBPF)Cl₂ (1.49 g, 2.29 mmol). The resulting mixture was stirred at 80° C. for 2 h under nitrogen atmosphere. The reaction mixture was quenched with water and extracted with ethyl acetate. The combined organic layers were washed with brine, dried over anhydrous sodium sulfate, filtered, and concentrated under vacuum. The residue was purified by flash chromatography on silica gel with 0˜50% ethyl acetate in petroleum ether to afford benzyl 2′-chloro-5′-methoxy-6-methyl-(4,4′-bipyridine)-3-carboxylate (4 g, 47.3%) as a yellow oil. MS (ESI) calc'd for (C₂₀H₁₇ClN₂O₃) (M+1)⁺, 369.1. found 369.0.

### Step 3 benzyl 5′-methoxy-2′,6-dimethyl-(4,4′-bipyridine)-3-carboxylate

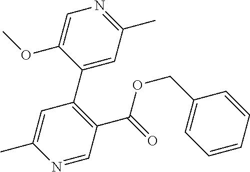 [SMILES: COc1cnc(C)cc1-c1cc(C)ncc1C(=O)OCc1ccccc1]

To a degassed mixture of benzyl 2-chloro-5-methoxy-6-methyl-(4,4-bipyridine)-3-carboxylate (4.00 g, 10.845 mmol) and K₂CO₃ (4.50 g, 33.0 mmol) in DME (30 mL) were added Pd(dppf)Cl₂ (0.79 g, 1.0 mmol) and trimethyl-1,3,5,2,4,6-trioxatriborinane (1.50 g, 12.0 mmol). The resulting mixture was stirred at 120° C. for 2 h under nitrogen atmosphere. The reaction mixture was quenched with water and extracted with ethyl acetate. The combined organic layers were washed with brine, dried over anhydrous sodium sulfate, filtered, and concentrated under vacuum. The residue was purified by reverse phase flash chromatography with 5˜70% acetonitrile in water to afford benzyl 5′-methoxy-2′,6-dimethyl-(4,4′-bipyridine)-3-carboxylate (2.8 g, 74.1%) as a yellow oil. MS (ESI) calc'd for (C₂₁H₂₀N₂O₃) (M+1)⁺, 349.1. found 349.0.

### Step 4 5′-methoxy-2′,6-dimethyl-(4,4′-bipyridine)-3-carboxylic Acid

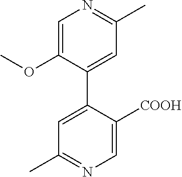 [SMILES: COc1cnc(C)cc1-c1cc(C)ncc1C(=O)O]

To a mixture of benzyl 5-methoxy-2,6-dimethyl-(4,4-bipyridine)-3-carboxylate (2.80 g, 8.037 mmol) in THF (20.00 mL) were added Pd/C (2.80 g, 10%). The resulting mixture was stirred at room temperature for 1 h under hydrogen atmosphere. The resulting mixture was filtered. The filtrate was concentrated vacuum to afford 5′-methoxy-2′,6-dimethyl-(4,4′-bipyridine)-3-carboxylic acid (2.5 g, cured) as a yellow solid, which was used for the next step directly without further purification. MS (ESI) calc'd for (C₁₄H₁₄N₂O₃) (M+1)⁺, 259.1. found 259.0.

### Intermediate G

### 2-chloro-5-methoxy-6-methyl-(4,4-bipyridine)-3-carboxylic Acid

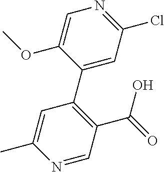 [SMILES: COc1cnc(Cl)cc1-c1cc(C)ncc1C(=O)O]

### Step-1: 2-chloro-5-methoxypyridin-4-ylboronic Acid

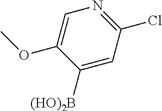

A stirred solution of 2-chloro-5-methoxypyridine (10.0 g, 69.65 mmol) in THF (500 mL) was added LDA (14.9 g, 139.30 mmol) dropwise at −78° C. under N₂ atmosphere. The resulting mixture was stirred at −78° C. for 2 h. Then Triisopropyl borate (26.2 g, 139.30 mmol) was added to the above mixture at −78° C. The resulting mixture was stirred at −78° C. for 2 h. Then the resulting mixture was stirred at room temperature for 16 h. The resulting mixture was quenched with HCl (2 N) and stirred at room temperature for 30 min. The resulting mixture was extracted with ethyl acetate. The reaction mixture was diluted with water and extracted with ethyl acetate. The combined organic layers were washed with brine, dried over anhydrous sodium sulfate, filtered, and concentrated under vacuum. 2-chloro-5-methoxypyridin-4-ylboronic acid (9 g, 68.9%) as a brown solid. MS (ESI) calc'd for (C₆H₇BClNO₃) (M+1)⁺, 188.0. found 188.0.

### Step-2: methyl 2-chloro-5-methoxy-6-methyl-(4,4-bipyridine)-3-carboxylate

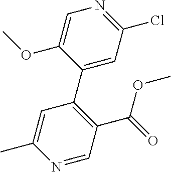

To a degassed solution of methyl 4-chloro-6-methylpyridine-3-carboxylate (700 mg, 3.77 mmol) and 2-chloro-5-methoxypyridin-4-ylboronic acid (918 mg, 4.90 mmol) in dioxane (6 mL) and H₂O (2 mL) were added Pd(dppf)Cl₂ (275 mg, 0.37 mmol) and K₂CO₃ (1563 mg, 11.31 mmol) under nitrogen atmosphere. The resulting mixture was stirred at 80° C. for 16 h under nitrogen atmosphere. The resulting mixture was filtered and the filtrate was concentrated under reduced pressure. The residue was purified by flash column chromatography with 0˜60% ethyl acetate in petroleum ether to afford methyl 2-chloro-5-methoxy-6-methyl-(4,4-bipyridine)-3-carboxylate (220 mg, 19.9%) as a white solid. MS (ESI) calc'd for (C₁₄H₁₃ClN₂O₃) (M+1)⁺, 293.1. found 293.1.

### Step-3: 2-chloro-5-methoxy-6-methyl-(4,4-bipyridine)-3-carboxylic Acid

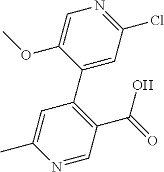 [SMILES: COc1cnc(Cl)cc1-c1cc(C)ncc1C(=O)O]

To a stirred solution of methyl 2-chloro-5-methoxy-6-methyl-(4,4-bipyridine)-3-carboxylate (220 mg, 0.75 mmol) in THF (2 mL) and water (2 mL) were added LiOH·H₂O (126 mg, 3.01 mmol). The resulting mixture was stirred at room temperature for 2 h. The mixture was acidified to pH 3 with citric acid. The resulting mixture was extracted with ethyl acetate. The combined organic layers were washed with brine, dried over anhydrous sodium sulfate, filtered, and concentrated under vacuum to afford 2-chloro-5-methoxy-6-methyl-(4,4-bipyridine)-3-carboxylic acid (160 mg, 76.3%) as a white solid. MS (ESI) calc'd for (C₁₃H₁₁ClN₂O₃) (M+1)⁺, 279.0. found, 279.0.

### Example 1

### 3-(2-methoxyphenyl)-N-(5-(prop-2-yn-1-yloxy)-1,3,4-thiadiazol-2-yl)isonicotinamide

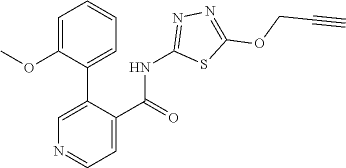 [SMILES: C#CCOc1nnc(NC(=O)c2ccncc2-c2ccccc2OC)s1]

### Step 1 S-methyl O-(prop-2-yn-1-yl) carbonodithioate

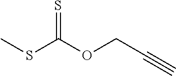

To a stirred solution of propargyl alcohol (15.00 g, 267.551 mmol, 1.00 equiv) in THF (200 mL) was added NaH (12.84 g, 321.062 mmol, 1.20 equiv, 60%) in portions at 0 degrees C. under nitrogen atmosphere. The resulting mixture was stirred for 30 min at 0 degrees C. under nitrogen atmosphere. To the above mixture was added CS₂ (24.45 g, 0.321 mmol, 1.2 equiv) dropwise at 0 degrees C. The resulting mixture was stirred for additional 10 min at 0 degrees C. followed by MeI (45.57 g, 0.321 mmol, 1.2 equiv). The resulting mixture was stirred for 10 min at 0 degrees C. The reaction was quenched by the addition 100 mL of NH₄Cl (aq.) at room temperature. The aqueous layer was extracted with EtOAc (2×2 100 mL). The residue was purified by silica gel column chromatography, eluted with PE/EA (20:1) to afford (methylsulfanyl)(prop-2-yn-1-yloxy)methanethione as a brown oil.

### Step 2 O-(prop-2-yn-1-yl) hydrazinecarbothioate

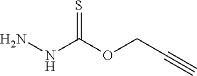

To a stirred solution/mixture of (methylsulfanyl)(prop-2-yn-1-yloxy)methanethione (25.00 g, 170.975 mmol, 1.00 equiv) in MeOH were added (methylsulfanyl)(prop-2-yn-1-yloxy)methanethione (25.00 g, 170.975 mmol, 1.00 equiv) dropwise at 0 degrees C. under nitrogen atmosphere. The resulting mixture was stirred for 30 min at room temperature. The resulting mixture was concentrated under reduced pressure. The crude product was used in the next step directly without further purification. This resulted in (((prop-2-yn-1-yloxy)methanethioyl)amino)amine (23 g, 95.08%) as a brown oil.

### Step 3 5-(prop-2-yn-1-yloxy)-1,3,4-thiadiazol-2-amine

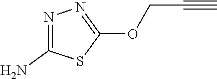

To a stirred solution of (((prop-2-yn-1-yloxy)methanethioyl)amino)amine (23.00 g, 176.692 mmol, 1.00 equiv) and TEA (35.76 g, 353.384 mmol, 2 equiv) in MeOH was added BrCN (22.46 g, 212.030 mmol, 1.2 equiv) in portions at 0 degrees C. under nitrogen atmosphere. The resulting mixture was stirred for 2 h at room temperature. The resulting mixture was diluted with water (200 mL). The resulting mixture was extracted with EtOAc (2×100 mL). The combined organic layers were dried over anhydrous Na2SO4. After filtration, the filtrate was concentrated under reduced pressure. The product was precipitated by the addition of EtOAc. This resulted in 5-(prop-2-yn-1-yloxy)-1,3,4-thiadiazol-2-amine (7.8 g, 26.46%) as an off-white solid.

### Step 4: 3-(2-methoxyphenyl)-N-(5-(prop-2-yn-1-yloxy)-1,3,4-thiadiazol-2-yl)isonicotinamide

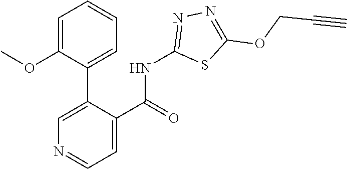 [SMILES: C#CCOc1nnc(NC(=O)c2ccncc2-c2ccccc2OC)s1]

To a stirred solution of Intermediate A (5.00 g, 0.22 mmol, 1.00 equiv) and DIEA (8.45 g, 0.65 mmol, 3.00 equiv) in DMF (150 mL) was added HATU (9.94 mg, 0.26 mmol, 1.20 equiv) at room temperature under nitrogen atmosphere. The resulting mixture was stirred for 1 h at room temperature. To the above mixture was added 5-(prop-2-yn-1-yloxy)-1,3,4-thiadiazol-2-amine (3.38 g, 0.22 mmol, 1.00 equiv) at room temperature. The resulting mixture was stirred for additional 2 h at room temperature. The resulting mixture was diluted with water (450 mL). The aqueous layer was extracted with EtOAc (3×200 mL). The residue was purified by silica gel column chromatography, eluted with CH₂Cl₂/MeOH (40:1). The residue was purified by reverse flash chromatography with the following condition: column, C18 silica gel; mobile phase, MeCN in water, 0% to 50% gradient in 20 min; detector, UV 254 nm to afford 3-(2-methoxyphenyl)-N-(5-(prop-2-yn-1-yloxy)-1,3,4-thiadiazol-2-yl)isonicotinamide (5.01 g, 61.84%) as an orange solid.

LC-MS: m/z 367 (M+H)⁺; H-NMR: ¹H NMR (300 MHz, Methanol-d4) δ8.68-8.67 (d, 1H), 8.60 (s, 1H), 7.65-7.64 (d, 1H), 7.42-7.36 (m, 2H), 7.11-7.08 (m, 1H), 6.99-6.96 (d, 1H), 5.09 (s, 2H), 3.60 (s, 3H), 3.11 (s, 1H) ppm.

### Example 2

### 4-(2-fluoro-6-methoxyphenyl)-N-(5-methoxy-1,3,4-thiadiazol-2-yl)-6-methylpyridine-3-carboxamide

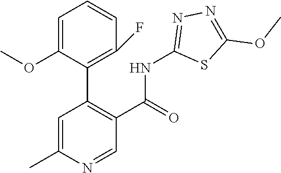

To a mixture of 5-methoxy-1,3,4-thiadiazol-2-amine (33.1 mg, 0.25 mmol) in acetonitrile (2 mL) were added Intermediate E (55.0 mg, 0.21 mmol) and NMI (36.3 mg, 0.44 mmol). A solution of TCFH (64.9 mg, 0.23 mmol) in acetonitrile (1 mL) was added thereto dropwise under nitrogen. The mixture was stirred at 20° C. for 2 hr under nitrogen. The mixture was concentrated under vacuum. The residue was dissolved in DMF (1 mL) and purified by Combi Flash (Biotage Isolera Prime) which applied to a 20 g C18 column, eluted with 5˜60% acetonitrile in water to afford 5-methoxy-1,3,4-thiadiazol-2-yl)-6-methylpyridine-3-carboxamide (47.1 mg, 58%) as a white solid. MS (ESI) calc'd for (C₁₇H₁₅FN₄O₃S) (M+1)⁺, 375.1. found 375.1. ¹H NMR (400 MHz, DMSO-d₆) δ 12.87 (s, 1H), 8.80 (s, 1H), 7.40-7.36 (m, 1H), 7.34-7.28 (m, 1H), 6.95-6.86 (m, 2H), 4.06 (s, 3H), 3.58 (s, 3H), 2.56 (s, 3H).

### Example 3

### 4-(2-fluoro-6-methoxyphenyl)-N-(5-hydroxy-1,3,4-thiadiazol-2-yl)-6-methylnicotinamide

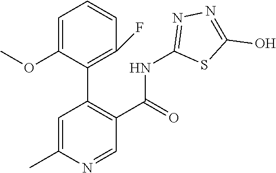 [SMILES: COc1cccc(F)c1-c1cc(C)ncc1C(=O)Nc1nnc(O)s1]

### Step-1: N-(5-((5-chloropyridin-2-yl)methoxy)-1,3,4-thiadiazol-2-yl)-4-(2-fluoro-6-methoxyphenyl)-6-methylnicotinamide

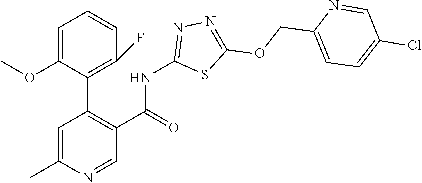 [SMILES: COc1cccc(F)c1-c1cc(C)ncc1C(=O)Nc1nnc(OCc2ccc(Cl)cn2)s1]

To a mixture of Intermediate B (111.4 mg, 0.46 mmol) in acetonitrile (2 mL) were added Intermediate E (100.0 mg, 0.38 mmol) and NMI (66.0 mg, 0.80 mmol). A solution of TCFH (118.1 mg, 0.42 mmol) in acetonitrile (1 mL) was added thereto dropwise under nitrogen. The mixture was stirred at 20° C. for 2 hr under nitrogen. The resulting mixture was concentrated under vacuum. The residue was dissolved in DMF (3 mL) and purified by Combi Flash (Biotage Isolera Prime) which applied to a 40 g C18 column, eluted with 15˜60% acetonitrile in water within 30 min to afford N-(5-((5-chloropyridin-2-yl)methoxy)-1,3,4-thiadiazol-2-yl)-4-(2-fluoro-6-methoxyphenyl)-6-methylnicotinamide (170.0 mg, 91%) as a white solid. MS (ESI) calc'd for (C₂₂H₁₇ClFN₅O₃S) (M+1)⁺, 486.1. found 486.0.

### Step-2: 4-(2-fluoro-6-methoxyphenyl)-N-(5-hydroxy-1,3,4-thiadiazol-2-yl)-6-methylnicotinamide

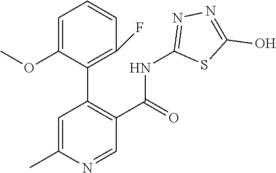 [SMILES: COc1cccc(F)c1-c1cc(C)ncc1C(=O)Nc1nnc(O)s1]

A mixture of N-(5-((5-chloropyridin-2-yl)methoxy)-1,3,4-thiadiazol-2-yl)-4-(2-fluoro-6-methoxyphenyl)-6-methylpyridine-3-carboxamide (170.0 mg, 0.35 mmol) in concentrated HCl (3 mL) was stirred at room temperature for 2 hr before concentrated under vacuum. The residue was basified with NH₃·H₂O to pH 9˜10. The aqueous solution was extracted with ethyl acetate. The combined organic layers were washed with brine, dried over anhydrous sodium sulfate, filtered, and concentrated under vacuum. The residue was purified by Prep-HPLC with the following condition: (Column: XBridge Prep OBD C18 Column, 30×150 mm 5 um; Mobile Phase A: Water (10 mmol/L NH₄HCO₃), Mobile Phase B: ACN; Flow rate: 60 mL/min; Gradient: 10 B to 40 B in 8 min; 220 nm; RT: 7.23 min) to afford 4-(2-fluoro-6-methoxyphenyl)-N-(5-hydroxy-1,3,4-thiadiazol-2-yl)-6-methylnicotinamide (56.4 mg, 44%) as a white solid. MS (ESI) calc'd for (C₁₆H₁₃FN₄O₃S) (M+1)⁺, 361.1. found 361.0. ¹H NMR (400 MHz, DMSO-d₆) δ 12.28 (s, 2H), 8.75 (s, 1H), 7.46-7.32 (m, 1H), 7.30 (s, 1H), 6.95-6.82 (t, J=8.0 Hz, 2H), 3.64 (s, 3H), 2.55 (s, 3H).

### Example 4

### 4-(2-fluoro-6-methoxyphenyl)-6-methyl-N-(5-(3,3,3-trifluoro-2,2-dimethylpropoxy)-1,3,4-thiadiazol-2-yl)pyridine-3-carboxamide

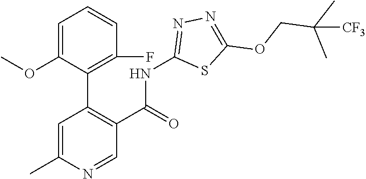 [SMILES: COc1cccc(F)c1-c1cc(C)ncc1C(=O)Nc1nnc(OCC(C)(C)C(F)(F)F)s1]

### Step 1 (methylsulfanyl)(3,3,3-trifluoro-2,2-dimethylpropoxy)methanethione

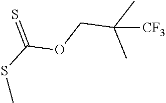

To a degassed solution of 3,3,3-trifluoro-2,2-dimethylpropan-1-ol (100.0 mg, 0.71 mmol) in dry Tetrahydrofuran (3 mL) was added NaH (34.0 mg, 1.41 mmol, 60%) in portions at 0° C. and stirred at 0° C. for 1 h under nitrogen atmosphere. Then CS₂ (81.0 mg, 1.00 mmol) was added to the above mixture at 0° C. for 20 min. Then MeI (150.0 mg, 1.05 mmol) was added to the above mixture at 0° C. for 30 min. Then the mixture resulting was stirred at room temperature for 2 hr under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (2 mL) which was applied to a 40 g C18 column and purified by Combi Flash (Biotage Isolera Prime), eluted with 5˜45% acetonitrile in water within 30 min to afford (methylsulfanyl)(3,3,3-trifluoro-2,2-dimethylpropoxy)methanethione (160.0 mg, 97%) as a white solid. MS (ESI) calc'd for (C₇H₁₁F₃N₂OS) (M+1)⁺, 234.0. found 234.0.

### Step 2 (((3,3,3-trifluoro-2,2-dimethylpropoxy)methanethioyl)amino)amine

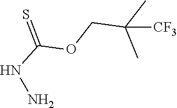

To a stirred solution of (methylsulfanyl)(3,3,3-trifluoro-2,2-dimethylpropoxy)methanethione (160.0 mg, 0.70 mmol) in MeOH (3 mL) was added NH₂NH₂·H₂O (34.0 mg, 0.70 mmol) dropwise at 0° C. under nitrogen atmosphere. The mixture resulting was stirred at 0° C. for 1 hr under nitrogen atmosphere. The resulting mixture was concentrated under vacuum to afford (((3,3,3-trifluoro-2,2-dimethylpropoxy)methanethioyl)amino)amine (150.0 mg, crude) as a yellow oil. MS (ESI) calc'd for (C₆H₁₁F₃N₂OS) (M+1)⁺, 217.1. found 217.1.

### Step 3 5-(3,3,3-trifluoro-2,2-dimethylpropoxy)-1,3,4-thiadiazol-2-amine

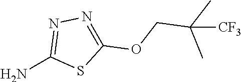

To a stirred solution of (((3,3,3-trifluoro-2,2-dimethylpropoxy)methanethioyl)amino)amine (150.0 mg, 0.70 mmol) in MeOH (3 mL) were added TEA (140.0 mg, 1.39 mmol) and BrCN (81.0 mg, 0.76 mmol) at 0° C. under nitrogen atmosphere. The mixture resulting was stirred at 0° C. for 1 hr under nitrogen atmosphere. The reaction mixture was quenched by the addition of water and extracted with ethyl acetate. The combined organic layers were washed with brine, dried over anhydrous sodium sulfate, filtered, and concentrated under vacuum to afford 5-(3,3,3-trifluoro-2,2-dimethylpropoxy)-1,3,4-thiadiazol-2-amine (140.0 mg, crude) as a pink solid. MS (ESI) calc'd for (C₇H₁₀F₃N₃OS) (M+1)⁺, 242.1. found 242.1.

### Step 4 4-(2-fluoro-6-methoxyphenyl)-6-methyl-N-(5-(3,3,3-trifluoro-2,2-dimethylpropoxy)-1,3,4-thiadiazol-2-yl)pyridine-3-carboxamide

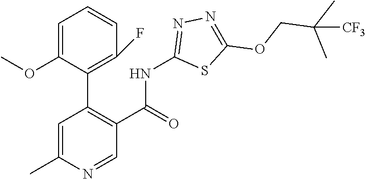 [SMILES: COc1cccc(F)c1-c1cc(C)ncc1C(=O)Nc1nnc(OCC(C)(C)C(F)(F)F)s1]

To a stirred solution of 5-(3,3,3-trifluoro-2,2-dimethylpropoxy)-1,3,4-thiadiazol-2-amine (50.0 mg, 0.21 mmol) and Intermediate E (54.0 mg, 0.21 mmol) in DMF (1 mL) were added NMI (68.0 mg, 0.83 mmol) and TCFH (87.0 mg, 0.31 mmol) at 25° C. under nitrogen atmosphere. The mixture was stirred at 25° C. for 1 hr under nitrogen atmosphere. The resulting mixture was purified by prep-HPLC with the following condition: (Column: XBridge Prep OBD C18 Column, 30×150 mm 5 um; Mobile Phase A: Water (10 mmol/L NH₄HCO₃), Mobile Phase B: ACN; Flow rate: 60 mL/min; Gradient: 42% B to 57% B in 8 min, UV: 254 nm) to afford 4-(2-fluoro-6-methoxyphenyl)-6-methyl-N-(5-(3,3,3-trifluoro-2,2-dimethylpropoxy)-1,3,4-thiadiazol-2-yl)pyridine-3-carboxamide (23.2 mg, 23%) as a white solid. MS (ESI) calc'd for (C₂₁H₂₀F₄N₄O₃S) (M+1)⁺, 485.1. found, 485.3. ¹H NMR (400 MHz, DMSO-d₆) δ 12.87 (s, 1H), 8.83 (s, 1H), 7.42-7.37 (m, 1H), 7.31 (d, J=1.6 Hz, 1H), 6.93-6.87 (m, 2H), 4.45 (s, 2H), 3.58 (s, 3H), 2.57 (s, 3H), 1.22 (s, 6H).

### Example 5

### 2′-chloro-5′-methoxy-N-(5-methoxy-1,3,4-thiadiazol-2-yl)-6-methyl-(4,4′-bipyridine)-3-carboxamide

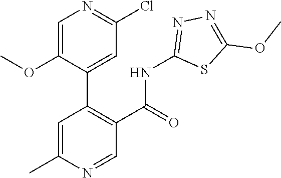

To a mixture of Intermediate G (50.0 mg, 0.17 mmol) in MeCN (1 mL) were added 5-methoxy-1,3,4-thiadiazol-2-amine (28.2 mg, 0.21 mmol) and NMI (30.9 mg, 0.37 mmol). A solution of TCFH (55.3 mg, 0.19 mmol) in MeCN (1 mL) was added thereto dropwise under nitrogen. The mixture was stirred at 20° C. for 2 hr under nitrogen. The resulting mixture was concentrated under vacuum. The residue was dissolved in DMF (3 mL) and purified by Combi Flash (Biotage Isolera Prime) which applied to a 40 g C18 column, eluted with 10˜60% acetonitrile in water within 30 min to afford 2′-chloro-5′-methoxy-N-(5-methoxy-1,3,4-thiadiazol-2-yl)-6-methyl-(4,4′-bipyridine)-3-carboxamide (34.7 mg, 33%) as a white solid. MS (ESI) calc'd for (C₁₆H₁₄ClN₅O₃S) (M+1)⁺, 392.0. found 392.1. ¹H NMR (400 MHz, DMSO-d₆) δ 12.93 (s, 1H), 8.81 (s, 1H), 8.17 (s, 1H), 7.55 (s, 1H), 7.44 (s, 1H), 4.08 (s, 3H), 3.63 (s, 3H), 2.59 (s, 3H).

### Example 6

### 2′-chloro-N-(5-isopropoxy-1,3,4-thiadiazol-2-yl)-5′-methoxy-6-methyl-(4,4′-bipyridine)-3-carboxamide

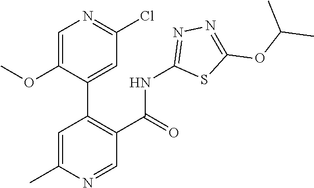 [SMILES: COc1cnc(Cl)cc1-c1cc(C)ncc1C(=O)Nc1nnc(OC(C)C)s1]

### Step-1: ((isopropoxymethanethioyl)amino)amine

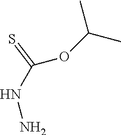

A mixture of isopropoxy(potassiosulfanyl)methanethione (1.0 g, 5.73 mmol) and Hydrazine (200.0 mg, 5.61 mmol) in MeOH (10 mL) was stirred at room temperature for 2 hr. The mixture was concentrated under vacuum to afford ((isopropoxymethanethioyl)amino)amine (800.0 mg, crude) as a yellow solid, which was used for the next step without further purification.

### Step-2: 5-isopropoxy-1,3,4-thiadiazol-2-amine

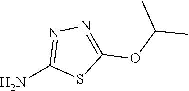

To a mixture of ((isopropoxymethanethioyl)amino)amine (400.0 mg, 2.98 mmol), TEA (603.2 mg, 5.96 mmol) in MeOH (20 mL) was added BrCN (173.6 mg, 1.63 mmol). The resulting mixture was stirred at room temperature for 2 hr. The reaction mixture was quenched with water and extracted with ethyl acetate. The combined organic layers were washed with brine, dried over anhydrous sodium sulfate, filtered, and concentrated under vacuum. The residue was dissolved in DMF (3 mL) and purified by Combi Flash (Biotage Isolera Prime) which applied to a 40 g C18 column, eluted with 15˜90% acetonitrile in water within 30 min to afford 5-isopropoxy-1,3,4-thiadiazol-2-amine (79.0 mg, 16%) as a white solid. MS (ESI) calc'd for (C₅H₉N₃OS) (M+1)⁺, 160.0. found 160.0.

### Step-3: 2′-chloro-N-(5-isopropoxy-1,3,4-thiadiazol-2-yl)-5′-methoxy-6-methyl-(4,4′-bipyridine)-3-carboxamide

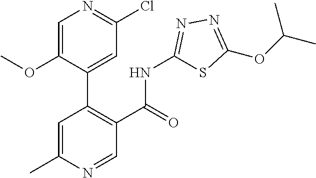 [SMILES: COc1cnc(Cl)cc1-c1cc(C)ncc1C(=O)Nc1nnc(OC(C)C)s1]

A mixture of Intermediate G (80.0 mg, 0.28 mmol) in MeCN (3 mL) were added 5-isopropoxy-1,3,4-thiadiazol-2-amine (59.4 mg, 0.37 mmol) and NMI (49.4 mg, 0.60 mmol). A solution of TCFH (88.6 mg, 0.31 mmol) in MeCN (1 mL) was added thereto dropwise under nitrogen. The resulting mixture was stirred at room temperature for 2 hr. The resulting mixture was concentrated under vacuum. The residue was dissolved in DMF (3 mL) and purified by Combi Flash (Biotage Isolera Prime) which applied to a 40 g C18 column, eluted with 15˜65% acetonitrile in water within 30 min to afford 2′-chloro-N-(5-isopropoxy-1,3,4-thiadiazol-2-yl)-5′-methoxy-6-methyl-(4,4′-bipyridine)-3-carboxamide (29.4 mg, 23%) as a white solid. MS (ESI) calc'd for (C₁₈H₁₈ClN₅O₃S) (M+1)⁺, 420.0. found 420.1. ¹H NMR (400 MHz, DMSO-d₆) δ 12.88 (s, 1H), 8.81 (s, 1H), 8.17 (s, 1H), 7.53 (s, 1H), 7.41 (s, 1H), 5.17-5.05 (m, 1H), 3.63 (s, 3H), 2.58 (s, 3H), 1.37 (d, J=6.0 Hz, 6H).

### Example 7

### 2′-chloro-N-(5-cyclopropoxy-1,3,4-thiadiazol-2-yl)-5′-methoxy-6-methyl-(4,4′-bipyridine)-3-carboxamide

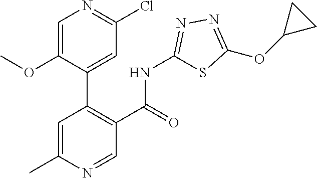 [SMILES: COc1cnc(Cl)cc1-c1cc(C)ncc1C(=O)Nc1nnc(OC2CC2)s1]

### Step 1 cyclopropoxy(methylsulfanyl)methanethione

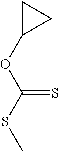

To a mixture of cyclopropanol (1.0 g, 17.21 mmol) in THF (20 mL) was added NaH (1.4 g, 34.40 mmol, 60%) in portions at 0° C. and stirred at 0° C. for 30 min. To the above mixture was added CS₂ (1.9 g, 25.82 mmol) at 0° C. and stirred at 0° C. for 30 min. Then MeI (3.7 g, 25.82 mmol) was added to the above mixture at 0° C. The resulting solution was stirred at 0° C. for 30 min. The reaction mixture was quenched with water and extracted with ethyl acetate. The combined organic solution was dried over sodium sulfate, filtered, and concentrated under vacuum to afford cyclopropoxy(methylsulfanyl)methanethione (2.0 g, crude) as a yellow oil.

### Step 2 N-amino-1-cyclopropoxymethanethioamide

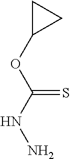

To a mixture of cyclopropoxy(methylsulfanyl)methanethione (1.5 g, 10.11 mmol) in MeOH (10 mL) was added Hydrazine (389.1 mg, 12.14 mmol). The mixture was stirred at 25° C. for 2 hr. The mixture was diluted with water (20 mL). The aqueous layer was extracted with ethyl acetate. The combined organic solution was dried over sodium sulfate, filtered, and concentrated under vacuum afford N-amino-1-cyclopropoxymethanethioamide (2.4 g, crude) as a yellow oil. MS (ESI) calc'd for (C₄H₈N₂OS) (M−1)⁺, 133.0. found 133.0.

### Step 3 5-cyclopropoxy-1,3,4-thiadiazol-2-amine

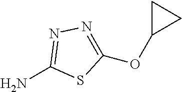

To a mixture of N-amino-1-cyclopropoxymethanethioamide (1.5 g, 11.34 mmol), TEA (2.3 g, 22.69 mmol) in MeOH (10 mL) were added BrCN (1.3 g, 12.48 mmol), the resulting solution was stirred at 25° C. for 30 min. The mixture was quenched with water. The aqueous layer was extracted with ethyl acetate. The combined organic solution was dried over sodium sulfate, filtered, and concentrated under vacuum to afford crude. The residue was dissolved in DMF (3 mL) and purified by Combi Flash (Biotage Isolera Prime) which applied to a 40 g C18 column, eluted with 15˜90% acetonitrile in water within 30 min to afford 5-cyclopropoxy-1,3,4-thiadiazol-2-amine (400.0 mg, 22% over three steps) as a yellow solid. MS (ESI) calc'd for (C₅H₇N₃OS) (M+1)⁺, 158.0. found 158.0.

### Step 4 2′-chloro-N-(5-cyclopropoxy-1,3,4-thiadiazol-2-yl)-5′-methoxy-6-methyl-(4,4′-bipyridine)-3-carboxamide

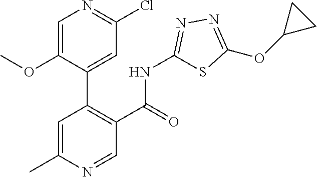 [SMILES: COc1cnc(Cl)cc1-c1cc(C)ncc1C(=O)Nc1nnc(OC2CC2)s1]

To a mixture of Intermediate G (100.0 mg, 0.35 mmol) in DMF (5 mL) were added 5-cyclopropoxy-1,3,4-thiadiazol-2-amine (84.6 mg, 0.53 mmol), HATU (204.6 mg, 0.53 mmol) and DIEA (139.1 mg, 1.07 mmol). The resulting solution was stirred at 25° C. for 2 hr. The resulting mixture was purified by Prep-HPLC with the following condition: (Column: YMC-Actus Triart C18 ExRS, 30 mm×150 mm, 5 um; Mobile Phase A: Water (10 MMOL/L NH₄HCO₃), Mobile Phase B: ACN; Flow rate: 60 mL/min; Gradient: 25% B to 40% B in 8 min, 40% B to 95% B in 9 min, 95% B to 95% B in 9.5 min, 95% B to 5% B in 10 min; Wave Length: 254 nm; RT (min): 7.80) to afford 2′-chloro-N-(5-cyclopropoxy-1,3,4-thiadiazol-2-yl)-5-methoxy-6-methyl-(4,4′-bipyridine)-3-carboxamide (54.6 mg, 36%) as a white solid. MS (ESI) calc'd for (C₁₈H₁₆ClN₅O₃S) (M+1)⁺, 418.0. found 418.0. ¹H NMR (400 MHz, DMSO-d₆) δ 12.90 (s, 1H), 8.83 (s, 1H), 8.17 (s, 1H), 7.51 (s, 1H), 7.40 (s, 1H), 4.39-4.29 (m, 1H), 3.64 (s, 3H), 2.59 (s, 3H), 0.91-0.77 (m, 4H).

### Example 8

### 5′-methoxy-N-(5-methoxy-1,3,4-thiadiazol-2-yl)-2′,6-dimethyl-(4,4′-bipyridine)-3-carboxamide

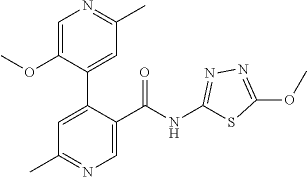

To a mixture of 5-methoxy-1,3,4-thiadiazol-2-amine (60.9 mg, 0.46 mmol) in DMF (1 mL) and acetonitrile (1 mL) were added Intermediate F (100.0 mg, 0.38 mmol), NMI (95.0 mg, 1.16 mmol). A solution of TCFH (163.0 mg, 0.58 mmol) in Acetonitrile (1 mL) was added thereto dropwise under nitrogen. The mixture was stirred at 20° C. for 2 hr under nitrogen. The resulting solution was purified by Combi Flash (Biotage Isolera Prime) which applied to a 40 g C18 column that was eluted with 5˜30% acetonitrile in water within 30 min to afford 5′-methoxy-N-(5-methoxy-1,3,4-thiadiazol-2-yl)-2′,6-dimethyl-(4,4′-bipyridine)-3-carboxamide (124.0 mg, 85%) as a white solid. MS (ESI) calculated for (C₁₇H₁₇N₅O₃S) (M+1)⁺, 372.1. found, 372.1. ¹H NMR (400 MHz, DMSO-d₆) δ 12.85 (s, 1H), 8.75 (s, 1H), 8.19 (s, 1H), 7.36 (s, 1H), 7.26 (s, 1H), 4.08 (s, 3H), 3.58 (s, 3H), 2.59 (s, 3H), 2.48 (s, 3H).

### Example 9

### 4-(5-chloro-2-methoxyphenyl)-N-(5-methoxy-1,3,4-thiadiazol-2-yl)-6-methylnicotinamide

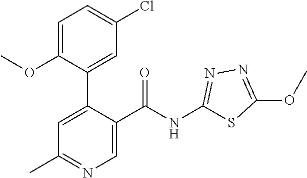

### Step 1 4-(5-chloro-2-methoxyphenyl)-6-methylpyridine-3-carboxylate

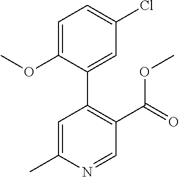

To a stirred solution of methyl 4-chloro-6-methylpyridine-3-carboxylate (600.0 mg, 3.23 mmol) and 5-chloro-2-methoxyphenylboronic acid (905.0 mg, 4.85 mmol) in dioxane (6 mL) were added water (2 mL) and Pd(PPh₃)₄ (375.0 mg, 0.32 mmol) and K₂CO₃ (1.3 g, 9.72 mmol). The resulting mixture was stirred at 80° C. for 2 hr under nitrogen atmosphere before concentrated under vacuum. The crude residue was applied to a 40 g C18 column, purified by Combi Flash (Biotage Isolera Prime), and eluted with 5˜50% acetonitrile in water within 45 min to afford methyl 4-(5-chloro-2-methoxyphenyl)-6-methylpyridine-3-carboxylate (730.0 mg, 76%) as a yellow oil. MS (ESI) calculated for (C₁₅H₁₄ClNO₃) (M+1)⁺, 292.1. found, 292.0.

### Step 2 4-(5-chloro-2-methoxyphenyl)-6-methylpyridine-3-carboxylic Acid

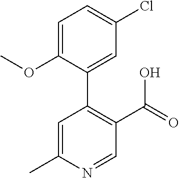 [SMILES: COc1ccc(Cl)cc1-c1cc(C)ncc1C(=O)O]

To a stirred solution of methyl 4-(5-chloro-2-methoxyphenyl)-6-methylpyridine-3-carboxylate (100.0 mg, 0.34 mmol) in tetrahydrofuran (1 mL) were added LiOH·H₂O (58.0 mg. 1.38 mmol) and water (0.3 mL). The resulting mixture was stirred at 25° C. for 1 hr. The residue was acidified to pH 7 with citric acid. The resulting mixture was extracted with ethyl acetate. The combined organic layers were washed with brine, dried over anhydrous Na₂SO₄. After filtration, the filtrate was concentrated under reduced pressure to afford 4-(5-chloro-2-methoxyphenyl)-6-methylpyridine-3-carboxylic acid (93.0 mg, crude) as a white solid. MS (ESI) calculated for (C₁₄H₁₂ClNO₃) (M+1)⁺, 278.1. found, 278.1.

### Step 3 4-(5-chloro-2-methoxyphenyl)-N-(5-methoxy-1,3,4-thiadiazol-2-yl)-6-methylnicotinamide

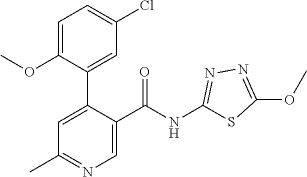

To a mixture of 5-methoxy-1,3,4-thiadiazol-2-amine (57.0 mg, 0.43 mmol) in acetonitrile (3 mL) were added 4-(5-chloro-2-methoxyphenyl)-6-methylnicotinic acid (93.0 mg, 0.36 mmol) and NMI (89.0 mg, 1.08 mmol). A solution of TCFH (111.0 mg, 0.39 mmol) in acetonitrile (1 mL) was added thereto dropwise under nitrogen. The mixture was stirred at 20° C. for 2 hr under nitrogen. The reaction mixture was applied to a 40 g C18 column, purified by Combi Flash (Biotage Isolera Prime), and eluted with 5˜40% acetonitrile in water within 45 min to afford 4-(5-chloro-2-methoxyphenyl)-N-(5-methoxy-1,3,4-thiadiazol-2-yl)-6-methylnicotinamide (91.3 mg, 64%) as a white solid. MS (ESI) calc'd for (C₁₇H₁₅ClN₄O₃S) (M+1)⁺, 391.0. found 391.1. ¹H NMR (400 MHz, DMSO-d₆) δ 12.75 (s, 1H), 8.70 (s, 1H), 7.48-7.40 (m, 2H), 7.35 (s, 1H), 7.01 (d, J=8.0 Hz, 1H), 4.07 (s, 3H), 3.50 (s, 3H), 2.57 (s, 3H).

### Example 10

### 2′-chloro-N-(5-(2-cyano-2-methylpropoxy)-1,3,4-thiadiazol-2-yl)-5′-methoxy-6-methyl-(4,4′-bipyridine)-3-carboxamide

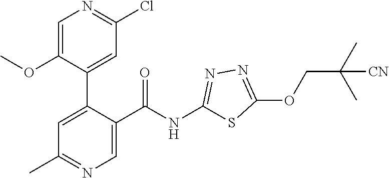 [SMILES: COc1cnc(Cl)cc1-c1cc(C)ncc1C(=O)Nc1nnc(OCC(C)(C)C#N)s1]

### Step-1: 3-((5-amino-1,3,4-thiadiazol-2-yl)oxy)-2,2-dimethylpropanenitrile

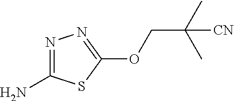

To a solution of NaH (303.0 mg, 7.57 mmol, 60%) in Tetrahydrofuran (5 mL) was added a solution of 3-hydroxy-2,2-dimethylpropanenitrile (500.0 mg, 5.04 mmol) in Tetrahydrofuran (2 mL) dropwise at 0° C. and stirred at 0° C. for 30 min. To the above solution was added 5-bromo-1,3,4-thiadiazol-2-amine (908.0 mg, 5.04 mmol) at 0° C. under nitrogen. The resulting solution was then stirred at 0° C. for 1.5 hr. The reaction mixture was quenched by the addition of water. The resulting mixture was concentrated under vacuum. The residue was dissolved in DMF (5 mL) and purified by Combi Flash which applied to 80 g C18 column that was eluted with 5˜70% acetonitrile in water within 30 min to afford 3-((5-amino-1,3,4-thiadiazol-2-yl)oxy)-2,2-dimethylpropanenitrile (50.0 mg, 5%) as a yellow solid. MS (ESI) calc'd for (C₇H₁₀N₄OS) (M+1)⁺, 199.1. found 199.2.

### Step-2: 2′-chloro-N-(5-(2-cyano-2-methylpropoxy)-1,3,4-thiadiazol-2-yl)-5′-methoxy-6-methyl-(4,4′-bipyridine)-3-carboxamide

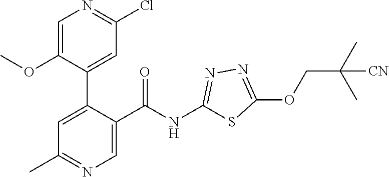 [SMILES: COc1cnc(Cl)cc1-c1cc(C)ncc1C(=O)Nc1nnc(OCC(C)(C)C#N)s1]

To a solution of Intermediate G (70.0 mg, 0.25 mmol) and 3-((5-amino-1,3,4-thiadiazol-2-yl)oxy)-2,2-dimethylpropanenitrile (50.0 mg, 0.25 mmol) in N,N-Dimethylformamide (2 mL) were added DIEA (98 mg, 0.76 mmol) and HATU (142.5 mg, 037 mmol) at 20° C. The resulting solution was stirred at 80° C. under nitrogen for 2 hr. The resulting mixture was applied to a 20 g C18 column, purified by Combi Flash (Biotage Isolera Prime), eluted with 5˜60% acetonitrile in water within 30 min. to afford 2′-chloro-N-(5-(2-cyano-2-methylpropoxy)-1,3,4-thiadiazol-2-yl)-5′-methoxy-6-methyl-(4,4′-bipyridine)-3-carboxamide (51.6 mg, 43%) as a white solid. MS (ESI) calc'd for (C₂₀H₁₉ClN₆O₃S) (M+1)⁺, 459.1. found, 459.0. 1H NMR (400 MHz, DMSO-d₆) δ 12.97 (s, 1H), 8.82 (s, 1H), 8.17 (s, 1H), 7.55 (s, 1H), 7.44 (s, 1H), 4.49 (s, 2H), 3.64 (s, 3H), 2.60 (s, 3H), 1.42 (s, 6H).

### Example 11

### 2′-chloro-N-(5-cyclobutoxy-1,3,4-thiadiazol-2-yl)-5′-methoxy-6-methyl-(4,4′-bipyridine)-3-carboxamide

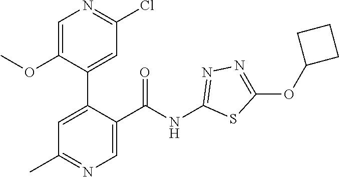 [SMILES: COc1cnc(Cl)cc1-c1cc(C)ncc1C(=O)Nc1nnc(OC2CCC2)s1]

### Step 1 5-cyclobutoxy-1,3,4-thiadiazol-2-amine

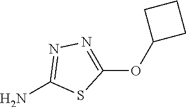 [SMILES: Nc1nnc(OC2CCC2)s1]

To a solution of NaH (416.0 mg, 10.40 mmol, 60%) in Tetrahydrofuran (THF) (5 mL) were added cyclobutanol (500.0 mg, 6.93 mmol) dropwise at 0° C. The resulting solution was then stirred at 0° C. for 30 min under nitrogen. To the above solution was added 5-bromo-1,3,4-thiadiazol-2-amine (1.3 g, 6.93 mmol) at 0° C. under nitrogen. The resulting solution was then stirred at 0° C. for 1 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (1 mL) and was applied to a 20 g silica gel column that was purified by Combi Flash (Biotage Isolera Prime), eluted with 0˜50% ethyl acetate in petroleum ether within 30 min to afford 5-cyclobutoxy-1,3,4-thiadiazol-2-amine (140.0 mg, 12%) as a yellow solid. MS (ESI) calc'd for (C₆H₉N₃OS) (M+1)⁺, 172.1. found 172.1.

### Step 2 2′-chloro-N-(5-cyclobutoxy-1,3,4-thiadiazol-2-yl)-5′-methoxy-6-methyl-(4,4′-bipyridine)-3-carboxamide

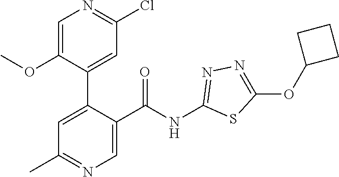 [SMILES: COc1cnc(Cl)cc1-c1cc(C)ncc1C(=O)Nc1nnc(OC2CCC2)s1]

To a solution of Intermediate G (146.0 mg, 0.53 mmol) in Acetonitrile (1 mL) were added 5-cyclobutoxy-1,3,4-thiadiazol-2-amine (90.0 mg, 0.53 mmol) and NMI (216.0 mg, 2.63 mmol). To the above was added TCFH (162.0 mg, 0.58 mmol) at 23° C. The resulting solution was stirred at 23° C. for 1 hr. The mixture was dissolved in DMF (1 mL) and was applied to a 20 g C18 column, purified by Combi Flash (Biotage Isolera Prime) that was eluted with 5˜50% acetonitrile in water within 30 min to afford 2′-chloro-N-(5-cyclobutoxy-1,3,4-thiadiazol-2-yl)-5′-methoxy-6-methyl-(4,4′-bipyridine)-3-carboxamide (24.7 mg, 11%) as a white solid. MS (ESI) calc'd for (C₁₉H₁₈ClN₅O₄S) (M+1)+432.1. found 432.1. ¹H NMR (400 MHz, DMSO-d₆) δ 12.83 (s, 1H), 8.79 (s, 1H), 8.17 (s, 1H), 7.53-7.34 (m, 2H), 5.22-5.16 (m, 1H), 3.63 (s, 3H), 2.59 (s, 3H), 2.59-2.18 (m, 4H), 1.77-1.85 (m, 1H), 1.71-1.53 (m, 1H).

### Example 12

### 2′-chloro-N-(5-(cyclohexyloxy)-1,3,4-thiadiazol-2-yl)-5′-methoxy-6-methyl-(4,4′-bipyridine)-3-carboxamide

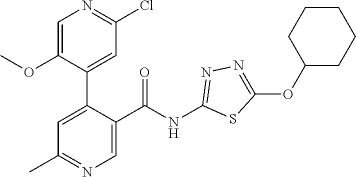 [SMILES: COc1cnc(Cl)cc1-c1cc(C)ncc1C(=O)Nc1nnc(OC2CCCCC2)s1]

### Step 1 O-cyclohexyl S-methyl Carbonodithioate

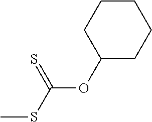

To a degassed mixture of NaH (200.0 mg, 4.99 mmol, 60%) in Tetrahydrofuran (6 mL) was added cyclohexanol (500.0 mg, 4.99 mmol) dropwise at 0° C. and stirred at 0° C. for 30 min. To the above solution was added CS₂ (570.0 mg, 7.49 mmol)) dropwise at 0° C. and stirred at 0° C. for 20 min. To the above solution was added MeI (1.1 g, 7.49 mmol) dropwise at 0° C. and stirred at 0° C. for 30 min. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (3 mL) and was applied to a 20 g silica gel column, purified by Combi Flash (Biotage Isolera Prime), eluted with 0˜10% ethyl acetate in petroleum ether within 20 min to afford O-cyclohexyl S-methyl carbonodithioate (400.0 mg, 42%) as a colorless oil. MS (ESI) calc'd for (C₈H₁₄OS) (M+1)⁺, 191.0. found, 191.1.

### Step 2 O-cyclohexyl Hydrazinecarbothioate

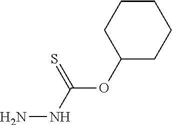 [SMILES: NNC(=S)OC1CCCCC1]

To a degassed solution of O-cyclohexyl S-methyl carbonodithioate (400.0 mg, 2.10 mmol) in Methanol (4 mL) was added hydrazine (126.1 mg, 3.15 mmol) at 0° C. under nitrogen atmosphere. The resulting solution was then stirred at 23° C. for 1 hr. The reaction mixture was diluted with water, extracted with ethyl acetate. The combined organic layers were washed with brine, dried over anhydrous sodium sulfate, filtered, and concentrated under vacuum to afford O-cyclohexyl hydrazinecarbothioate (320.1 mg, crude) as a white solid. MS (ESI) calc'd for (C₇H₁₄N₂OS) (M+1)⁺, 175.1. found, 175.1.

### Step 3 5-(cyclohexyloxy)-1,3,4-thiadiazol-2-amine

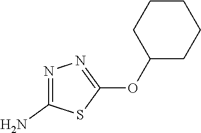 [SMILES: Nc1nnc(OC2CCCCC2)s1]

To a degassed solution of O-cyclohexyl hydrazinecarbothioate (320.0 mg, 1.84 mmol) in Methanol (4 mL) was added TEA (372.0 mg, 3.67 mmol) and BrCN (270.0 mg, 2.57 mmol) at 23° C. under nitrogen atmosphere. The resulting solution was stirred at 23° C. for 2 hr under nitrogen. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash which applied to 20 g silica gel column, purified by Combi Flash (Biotage Isolera Prime), eluted with 0˜30% ethyl acetate in petroleum ether within 25 min to afford 5-(cyclohexyloxy)-1,3,4-thiadiazol-2-amine (220.0 mg, 57%) as a white solid. MS (ESI) calc'd for (C₈H₁₃N₃OS) (M+1)⁺, 200.1. found 200.1.

### Step 4 2′-chloro-N-(5-(cyclohexyloxy)-1,3,4-thiadiazol-2-yl)-5′-methoxy-6-methyl-(4,4′-bipyridine)-3-carboxamide

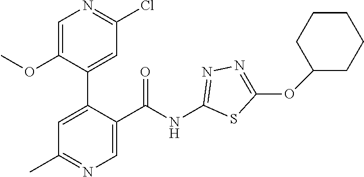 [SMILES: COc1cnc(Cl)cc1-c1cc(C)ncc1C(=O)Nc1nnc(OC2CCCCC2)s1]

To a solution of Intermediate G (280.1 mg, 1.01 mmol) in N,N-Dimethylformamide (2 mL) were added 5-(cyclohexyloxy)-1,3,4-thiadiazol-2-amine (200.0 mg, 1.01 mmol) and 1-methylimidazole (247.2 mg, 3.01 mmol). To the above was added a solution of TCFH (310.0 mg, 1.10 mmol) in Acetonitrile (1 mL) at 23° C. The resulting solution was stirred at 23° C. for 2 hr under nitrogen. The mixture was diluted with DMF (2 mL) which was applied to a 20 g C18 column and purified by Combi Flash (Biotage Isolera Prime), eluted with 5˜50% acetonitrile in water within 30 min to afford 2′-chloro-N-(5-(cyclohexyloxy)-1,3,4-thiadiazol-2-yl)-5′-methoxy-6-methyl-(4,4′-bipyridine)-3-carboxamide (240.1 mg, 52%) as a white solid. MS (ESI) calc'd for (C₂₁H₂₂ClN₅O₃S) (M+1)⁺, 460.1. found 460.1. ¹H NMR (400 MHz, DMSO-d₆) δ 12.89 (s, 1H), 8.80 (s, 1H), 8.18 (s, 1H), 7.54 (s, 1H), 7.43 (s, 1H), 4.94-4.83 (m, 1H), 3.63 (s, 3H), 2.59 (s, 3H), 2.10-1.99 (m, 2H), 1.75-1.67 (m, 2H), 1.56-1.49 (m, 3H), 1.45-1.23 (m, 3H).

### Example 13

### 2′-chloro-5′-methoxy-6-methyl-N-(5-(oxetan-3-yloxy)-1,3,4-thiadiazol-2-yl)-(4,4′-bipyridine)-3-carboxamide

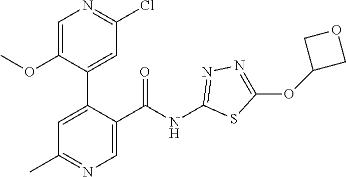 [SMILES: COc1cnc(Cl)cc1-c1cc(C)ncc1C(=O)Nc1nnc(OC2COC2)s1]

### Step 1 5-(oxetan-3-yloxy)-1,3,4-thiadiazol-2-amine

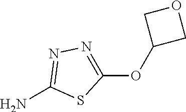 [SMILES: Nc1nnc(OC2COC2)s1]

To a degassed solution of NaH (405.2 mg, 10.12 mmol, 60%) in tetrahydrofuran (8 mL) was added oxetan-3-ol (500.1 mg, 6.75 mmol) at 0° C. and stirred at 0° C. for 30 min. To the above mixture was added 5-bromo-1,3,4-thiadiazol-2-amine (1.2 g, 6.75 mmol) under nitrogen. The resulting solution was then stirred at 0° C. for 1 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (1.5 mL), applied to a 40 g C18 column and purified by Combi Flash (Biotage Isolera Prime), eluted with 5˜20% acetonitrile in water within 20 min to afford 5-(oxetan-3-yloxy)-1,3,4-thiadiazol-2-amine (112.2 mg, 9%) as a yellow solid. MS (ESI) calc'd for (C₅H₇N₃O₂S) (M+1)⁺, 174.0. found 174.0.

### Step 2 2′-chloro-5′-methoxy-6-methyl-N-(5-(oxetan-3-yloxy)-1,3,4-thiadiazol-2-yl)-(4,4′-bipyridine)-3-carboxamide

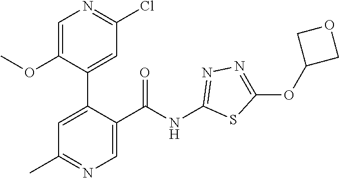 [SMILES: COc1cnc(Cl)cc1-c1cc(C)ncc1C(=O)Nc1nnc(OC2COC2)s1]

To a solution of 5-(oxetan-3-yloxy)-1,3,4-thiadiazol-2-amine (100.0 mg, 0.58 mmol) in N,N-dimethylformamide (1 mL) and acetonitrile (1 mL) were added Intermediate G (161.2 mg, 0.58 mmol) and 1-methylimidazole (142.2 mg, 1.73 mmol). To the above mixture was added a solution of TCFH (178.3 mg, 0.64 mmol) in Acetonitrile (1 mL) at 23° C. The mixture was stirred at 23° C. for 2 hr under nitrogen. The resulting mixture was dissolved in DMF (2 mL) which was applied to a 20 g C18 column and purified by Combi Flash (Biotage Isolera Prime), eluted with 5˜40% acetonitrile in water within 30 min to afford 2′-chloro-5′-methoxy-6-methyl-N-(5-(oxetan-3-yloxy)-1,3,4-thiadiazol-2-yl)-(4,4′-bipyridine)-3-carboxamide (65.3 mg, 25%) as a white solid. MS (ESI) calc'd for (C₁₈H₁₆ClN₅SO₄S) (M+1)⁺, 434.1. found, 434.0. 1H NMR (400 MHz, DMSO-d6) δ 13.01 (s, 1H), 8.80 (s, 1H), 8.18 (s, 1H), 7.55 (s, 1H), 7.44 (s, 1H), 5.72-5.62 (m, 1H), 4.94-4.86 (m, 2H), 4.69-4.61 (m, 2H), 3.63 (s, 3H), 2.59 (s, 3H).

### Example 14

### 2′-chloro-N-(5-(2-hydroxy-2-methylpropoxy)-1,3,4-thiadiazol-2-yl)-5′-methoxy-6-methyl-(4,4′-bipyridine)-3-carboxamide

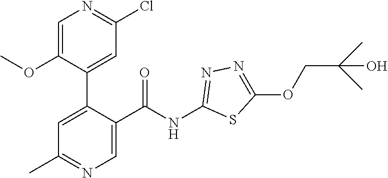 [SMILES: COc1cnc(Cl)cc1-c1cc(C)ncc1C(=O)Nc1nnc(OCC(C)(C)O)s1]

### Step-1: methyl 2-((5-amino-1,3,4-thiadiazol-2-yl)oxy)acetate

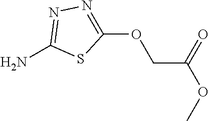

To a degassed solution of methyl 2-hydroxyacetate (3.0 g, 33.3 mmol) in Tetrahydrofuran (30 mL) was added NaH (2.0 g, 50.0 mmol, 60%) in portions at 0° C. The resulting solution was stirred at 0° C. for 1 hr under nitrogen. To the above solution was added 5-bromo-1,3,4-thiadiazol-2-amine (6.0 g, 33.3 mmol) at 0° C. The resulting solution was then stirred at 0° C. for 2 hr under nitrogen. The reaction mixture was quenched by the addition of water and extracted with ethyl acetate. The combined organic layers were washed with brine, dried over anhydrous sodium sulfate, filtered, and concentrated under vacuum. The resulting residue was purified by normal phase flash chromatography which applied to 40 g silica gel column, purified by Combi Flash (Biotage Isolera Prime) and eluted with 0˜66% ethyl acetate in petroleum ether within 30 min to afford methyl 2-((5-amino-1,3,4-thiadiazol-2-yl)oxy)acetate (418.0 mg, 6%) as a white solid. MS (ESI) calculated for (C₅H₇N₃O₃S) (M+1)⁺, 190.0. found, 190.0.

### Step-2: methyl 2-((5-(2′-chloro-5′-methoxy-6-methyl-(4,4′-bipyridine)-3-carboxamido)-1,3,4-thiadiazol-2-yl)oxy)acetate

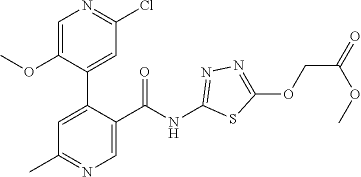 [SMILES: COC(=O)COc1nnc(NC(=O)c2cnc(C)cc2-c2cc(Cl)ncc2OC)s1]

To a stirred solution of methyl 2-((5-amino-1,3,4-thiadiazol-2-yl)oxy)acetate (200.0 mg, 1.06 mmol) in N,N-Dimethylformamide (2 mL) were sequentially added Intermediate G (295.0 mg, 1.06 mmol) and 1-methyl-1H-imidazole (347.0 mg, 4.23 mmol). A solution of TCFH (445.0 mg, 1.59 mmol) in acetonitrile (2 mL) was added thereto at 23° C. The mixture was stirred at 23° C. for 1 h. The resulting mixture was diluted in DMF (2 mL) and was applied to a 40 g C18 column, purified by Combi Flash (Biotage Isolera Prime) and eluted with 5˜34% acetonitrile in water within 30 min to afford methyl 2-((5-(2′-chloro-5′-methoxy-6-methyl-(4,4′-bipyridine)-3-carboxamido)-1,3,4-thiadiazol-2-yl)oxy)acetate (350.0 mg, 72%) as a white solid. MS (ESI) calculated for (C₁₈H₁₆ClN₅O₅S) (M+1)⁺, 450.1. found, 450.1.

### Step-3: 2′-chloro-N-(5-(2-hydroxy-2-methylpropoxy)-1,3,4-thiadiazol-2-yl)-5′-methoxy-6-methyl-(4,4′-bipyridine)-3-carboxamide

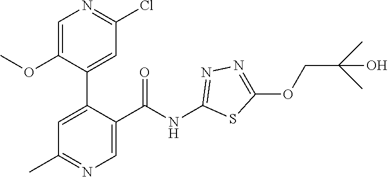 [SMILES: COc1cnc(Cl)cc1-c1cc(C)ncc1C(=O)Nc1nnc(OCC(C)(C)O)s1]

To a degassed solution of methyl methyl 2-((5-(2′-chloro-5′-methoxy-6-methyl-(4,4′-bipyridine)-3-carboxamido)-1,3,4-thiadiazol-2-yl)oxy)acetate (100.0 mg, 0.22 mmol) in dry Tetrahydrofuran (1 mL) was added methylmagnesium bromide (0.3 mL, 0.90 mmol, 3 N in THF) dropwise at 0° C. The mixture was stirred at 0° C. for 30 min under nitrogen atmosphere. The reaction mixture was quenched by the addition of water and extracted with ethyl acetate. The combined organic layers were washed with brine, dried over anhydrous sodium sulfate, filtered, and concentrated under vacuum. The residue was dissolved in Methanol (4 mL) and was purified by prep-HPLC with the following condition: (Column: Xbridge Prep OBD C18 Column, 30*150 mm, 5 um; Mobile Phase A: Water (10 mmol/L NH₄HCO₃), Mobile Phase B: MeOH-HPLC; Flow rate: 60 mL/min; Gradient: 38% B to 55% B in 8 min, 55% B to 95% B in 8.2 min, 95% B to 95% B in 10 min, 95% B to 38% B in 11 min, 38% B; Wave Length: 254/220 nm; RT1 (min): 7; Injection Volume: 0.8 mL; Number Of Runs: 5) to afford 2′-chloro-N-(5-(2-hydroxy-2-methylpropoxy)-1,3,4-thiadiazol-2-yl)-5′-methoxy-6-methyl-(4,4′-bipyridine)-3-carboxamide (41.7 mg, 41%) as a white solid. MS (ESI) calculated for (C₁₉H₂₀ClN₅O₄S) (M+1)⁺, 450.1. found, 450.1. ¹H NMR (400 MHz, DMSO-d₆) δ 12.87 (s, 1H), 8.82 (s, 1H), 8.17 (s, 1H), 7.52 (s, 1H), 7.41 (s, 1H), 4.77 (s, 1H), 4.18 (s, 2H), 3.64 (s, 3H), 2.59 (s, 3H), 1.18 (s, 6H).

### Example 15 and 16

### 2′-chloro-N-(5-((1s,3s)-3-hydroxycyclobutoxy)-1,3,4-thiadiazol-2-yl)-5′-methoxy-6-methyl-(4,4′-bipyridine)-3-carboxamide and 2′-chloro-N-(5-((1r,3r)-3-hydroxycyclobutoxy)-1,3,4-thiadiazol-2-yl)-5′-methoxy-6-methyl-(4,4′-bipyridine)-3-carboxamide

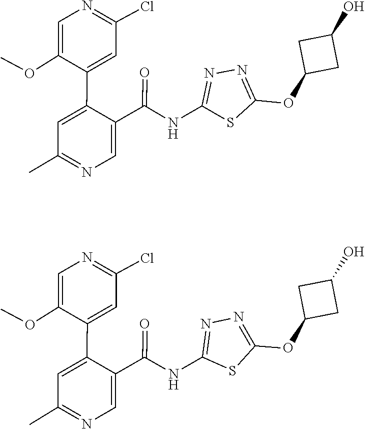 [SMILES: COc1cnc(Cl)cc1-c1cc(C)ncc1C(=O)Nc1nnc(O[C@H]2C[C@@H](O)C2)s1 ; COc1cnc(Cl)cc1-c1cc(C)ncc1C(=O)Nc1nnc(O[C@H]2C[C@H](O)C2)s1]

### Step 1 O-(3-(benzyloxy)cyclobutyl)S-methyl Carbonodithioate

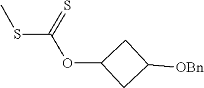 [SMILES: CSC(=S)OC1CC(OCc2ccccc2)C1]

To a solution of NaH (242 mg, 6.06 mmol, 60%) in THF (5 mL) was added a solution of 3-(benzyloxy)cyclobutan-1-ol (900 mg, 5.06 mmol) in THF (5 mL) dropwise at 0° C. The resulting mixture was stirred at 0° C. for 30 min. To the above mixture was added CS₂ (576 mg, 7.59 mmol) dropwise at 0° C. and stirred at 0° C. for 20 min. Then MeI (1.07 mg, 7.59 mmol) was added to the above mixture dropwise at 0° C. The resulting mixture was stirred at room temperature for 1 hour. The resulting mixture was quenched with water. The aqueous layer was extracted with ethyl acetate. The organic layers were combined, washed with brine, dried over anhydrous sodium sulfate and filtered. The filtrate was concentrated under vacuum. The resulting residue was dissolved in acyl acetate (5 mL) and purified by Combi Flash (Biotage Isolera Prime) which applied to a 40 g silica gel column that was eluted with 0˜82% ethyl acetate in petroleum ether within 35 min to afford O-(3-(benzyloxy)cyclobutyl) S-methyl carbonodithioate (940 mg, 69%) as a yellow oil. MS (ESI) calculated for (C₁₃H₁₆O₂S₂) (M+1)⁺, 269.06. found, 269.06.

### Step 2 O-(3-(benzyloxy)cyclobutyl) Hydrazinecarbothioate

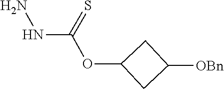 [SMILES: NNC(=S)OC1CC(OCc2ccccc2)C1]

To a mixture of S-methyl O-(3-(benzyloxy)cyclobutyl)S-methyl carbonodithioate (940 mg, 3.50 mmol) in MeOH (5 mL) was added Hydrazine (241 mg, 3.80 mmol 80%). The mixture was stirred at 0° C. for 1 hour. The resulting mixture was concentrated under vacuum and then diluted with water. The resulting mixture was extracted with ethyl acetate. The combined organic layers were washed with brine, dried over anhydrous sodium sulfate, filtered, and concentrated under vacuum to afford O-(3-(benzyloxy)cyclobutyl) hydrazinecarbothioate (830 mg, crude) as a yellow oil. MS (ESI) calculated for (C₁₂H₁₆N₂O₂S) (M+1)⁺, 253.09. found, 253.09.

### Step 3 5-(3-(benzyloxy)cyclobutoxy)-1,3,4-thiadiazol-2-amine

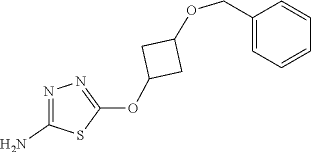 [SMILES: Nc1nnc(OC2CC(OCc3ccccc3)C2)s1]

To a mixture of O-(3-(benzyloxy)cyclobutyl) hydrazinecarbothioate (860 mg, 3.40 mmol) and Et₃N (689 mg, 6.81 mmol) in MeOH (5 mL) was added BrCN (394 mg, 3.74 mmol). The mixture was stirred at 23° C. for 1 h. The resulting mixture was quenched with water. The aqueous layer was extracted with ethyl acetate. The organic layers were combined, washed with brine, dried over anhydrous sodium sulfate and filtered. The resulting residue was purified by Combi Flash (Biotage Isolera Prime) which applied to 40 g silica gel column and eluted with 0˜55% ethyl acetate in petroleum ether within 35 min to afford 5-(3-(benzyloxy)cyclobutoxy)-1,3,4-thiadiazol-2-amine (410 mg, 43%) as a white solid. MS (ESI) calculated for (C₁₃H₁₅N₃O₂S) (M+1)⁺, 278.09. found, 278.09.

### Step 4 3-((5-amino-1,3,4-thiadiazol-2-yl)oxy)cyclobutan-1-ol

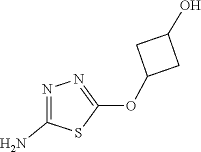 [SMILES: Nc1nnc(OC2CC(O)C2)s1]

To a stirred solution of 5-(3-(benzyloxy)cyclobutoxy)-1,3,4-thiadiazol-2-amine (320 mg, 1.154 mmol) in Dichloromethane (5 mL) was added BBr₃ (0.545 mL, 5.77 mmol) at −78° C. The resulting solution was stirred at −78° C. for 1 h. The reaction mixture was quenched with the addition of water and extracted with ethyl acetate. The combined organic layers were washed with water, dried over anhydrous Na₂SO₄ and concentrated under reduced pressure. The resulting residue was dissolved in DMF (3 mL) which was applied to a 20 g C18 column and purified by Combi Flash (Biotage Isolera Prime), eluted with 5˜35% acetonitrile in water within 25 min to afford 3-((5-amino-1,3,4-thiadiazol-2-yl)oxy)cyclobutan-1-ol (105 mg, 48%) as a white solid. MS (ESI) calculated for (C₆H₉N₃O₂S) (M+1)⁺, 188.04. found, 188.04.

### Step 5 2′-chloro-N-(5-(3-hydroxycyclobutoxy)-1,3,4-thiadiazol-2-yl)-5′-methoxy-6-methyl-(4,4′-bipyridine)-3-carboxamide

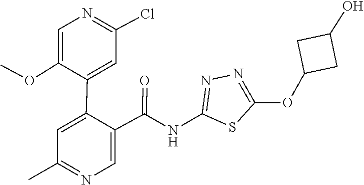 [SMILES: COc1cnc(Cl)cc1-c1cc(C)ncc1C(=O)Nc1nnc(OC2CC(O)C2)s1]

To a stirred solution of 3-((5-amino-1,3,4-thiadiazol-2-yl)oxy)cyclobutan-1-ol (100 mg, 0.534 mmol) in N,N-Dimethylformamide (0.5 mL) and Acetonitrile (0.5 mL) were added 1-methylimidazole (0.213 mL, 2.67 mmol), Intermediate G (149 mg, 0.534 mmol), and TCFH (165 mg, 0.588 mmol) at 23° C. The resulting solution was stirred at 23° C. for 1 h. The resulting residue was dissolved in DMF (0.5 mL) which was applied to a 20 g C18 column and purified by Combi Flash (Biotage Isolera Prime), eluted with 5˜42% acetonitrile in water within 25 min to afford 2′-chloro-N-(5-(3-hydroxycyclobutoxy)-1,3,4-thiadiazol-2-yl)-5′-methoxy-6-methyl-(4,4′-bipyridine)-3-carboxamide (50 mg, 19%) as a yellow solid. MS (ESI) calculated for (C₁₉H₁₈ClN₅O₄S) (M+1)⁺, 448.1. found, 448.1.

### Step 6 2′-chloro-N-(5-((1s,3s)-3-hydroxycyclobutoxy)-1,3,4-thiadiazol-2-yl)-5′-methoxy-6-methyl-(4,4′-bipyridine)-3-carboxamide and 2′-chloro-N-(5-((1r,3r)-3-hydroxycyclobutoxy)-1,3,4-thiadiazol-2-yl)-5′-methoxy-6-methyl-(4,4′-bipyridine)-3-carboxamide

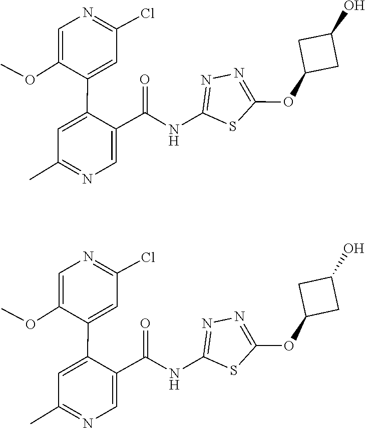 [SMILES: COc1cnc(Cl)cc1-c1cc(C)ncc1C(=O)Nc1nnc(O[C@H]2C[C@@H](O)C2)s1 ; COc1cnc(Cl)cc1-c1cc(C)ncc1C(=O)Nc1nnc(O[C@H]2C[C@H](O)C2)s1]

The mixture of compounds (50 mg) was dissolved in DMF (7 mL) which was purified by prep-HPLC with the following condition: (Column: XBridge Prep Phenyl OBD Column, 19*250 mm, 5 um; Mobile Phase A: Water (10 mmol/L NH₄HCO₃), Mobile Phase B: MeOH—Preparative; Flow rate: 25 mL/min; Gradient: 40% B to 50% B in 12 min, 50% B to 95% B in 12.2 min, 95% B to 95% B in 14 min, 95% B to 5% B in 14.2 min, 5% B to 5% B in 16 min; Wave Length: 254 nm; Injection Volume: 0.6 mL; Number Of Runs: 11) to afford 2′-chloro-N-(5-((1r,3r)-3-hydroxycyclobutoxy)-1,3,4-thiadiazol-2-yl)-5′-methoxy-6-methyl-(4,4′-bipyridine)-3-carboxamide (18.8 mg, 37.1% yield) as a white solid and 2′-chloro-N-(5-((1s,3s)-3-hydroxycyclobutoxy)-1,3,4-thiadiazol-2-yl)-5′-methoxy-6-methyl-(4,4′-bipyridine)-3-carboxamide (5.8 mg, 11.43% yield) as a white solid.

2′-chloro-N-(5-((1s,3s)-3-hydroxycyclobutoxy)-1,3,4-thiadiazol-2-yl)-5′-methoxy-6-methyl-(4,4′-bipyridine)-3-carboxamide: MS (ESI) calculated for (C₁₉H₁₈ClN₅O₄S) (M+1)⁺, 448.1. found, 448.1. ¹H NMR (400 MHz, DMSO-d₆) δ 12.88 (s, 1H), 8.81 (s, 1H), 8.17 (s, 1H), 7.53 (s, 1H), 7.42 (s, 1H), 5.24 (d, J=6.0 Hz, 1H), 4.72 (d, J=6.0 Hz, 1H), 3.83 (d, J=6.0 Hz, 1H), 3.63 (s, 3H), 2.82-2.76 (m, 2H), 2.59 (s, 3H), 2.10-1.96 (m, 2H).

2′-chloro-N-(5-((1r,3r)-3-hydroxycyclobutoxy)-1,3,4-thiadiazol-2-yl)-5′-methoxy-6-methyl-(4,4′-bipyridine)-3-carboxamide: MS (ESI) calculated for (C₁₉H₁₈ClN₅O₄S) (M+1)⁺, 448.1. found, 448.1. ¹H NMR (400 MHz, DMSO-d₆) δ 12.89 (s, 1H), 8.82 (s, 1H), 8.17 (s, 1H), 7.51 (s, 1H), 7.40 (s, 1H), 5.29-5.25 (m, 2H), 4.39 (d, J=6.0 Hz, 1H), 3.63 (s, 3H), 2.58 (s, 3H), 2.43-2.38 (m, 2H), 2.36-2.31 (m, 2H).

2′-chloro-N-(5-((1s,3s)-3-hydroxycyclobutoxy)-1,3,4-thiadiazol-2-yl)-5′-methoxy-6-methyl-(4,4′-bipyridine)-3-carboxamide was re-synthesized following the above procedure using (1s,3s)-3-(benzyloxy)cyclobutan-1-ol as starting material to confirm absolute stereochemistry.

### Example 17

### 2′-chloro-N-(5-(cyclopentyloxy)-1,3,4-thiadiazol-2-yl)-5′-methoxy-6-methyl-(4,4′-bipyridine)-3-carboxamide

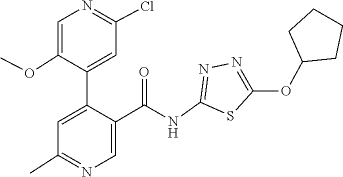 [SMILES: COc1cnc(Cl)cc1-c1cc(C)ncc1C(=O)Nc1nnc(OC2CCCC2)s1]

### Step 1 O-cyclopentyl S-methyl Carbonodithioate

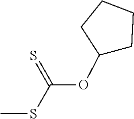

To a degassed solution of cyclopentanol (500 mg, 5.80 mmol) in dry Tetrahydrofuran (THF) (2 mL) was added NaH (464 mg, 11.61 mmol) in portions at 0° C. and stirred at 25° C. for 30 min. To the above solution were sequentially added CS₂ (0.525 mL, 8.71 mmol) and MeI (0.544 mL, 8.71 mmol) at 25° C. The resulting mixture was then stirred at 25° C. for 30 min. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (3 mL) and was applied to a 20 g silica gel column that was eluted with 0˜30% ethyl acetate in petroleum ether within 25 min to afford O-cyclopentyl S-methyl carbonodithioate (972 mg, 93% yield) as a colorless oil. MS (ESI) calc'd for (C₇H₁₂OS₂) (M+1)⁺, 177.0. found 177.0.

### Step 2 O-cyclopentyl Hydrazinecarbothioate

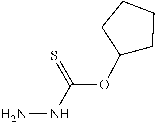

To a stirred solution of O-cyclopentyl S-methyl carbonodithioate (970 mg, 5.50 mmol) in Methanol (5 mL) was added N2H4·H2O (344 mg, 5.50 mmol) at 25° C. The resulting solution was stirred at 25° C. for 2 h. The reaction mixture was quenched by the addition of water and extracted with ethyl acetate. The combined organic layers were washed with brine, dried over anhydrous sodium sulfate, filtered, and concentrated under vacuum to afford O-cyclopentyl hydrazinecarbothioate (826 mg, crude) as a yellow oil. The crude procedure was used in the next step without further purification. MS (ESI) calc'd for (C₆H₁₂N₂OS) (M+1)⁺, 161.11. found 161.1.

### Step 3 5-(cyclopentyloxy)-1,3,4-thiadiazol-2-amine

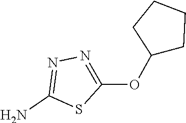 [SMILES: Nc1nnc(OC2CCCC2)s1]

To a stirred solution of O-cyclopentyl hydrazinecarbothioate (826 mg, 5.15 mmol) in Ethanol (5 mL) were added TEA (0.719 mL, 5.15 mmol) and BrCN (546 mg, 5.15 mmol) at 25° C. The resulting solution was stirred at 25° C. for 1 h. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (5 mL) and was applied to a 40 g silica gel column that was eluted with 0˜50% ethyl acetate in petroleum ether within 30 min to afford 5-(cyclopentyloxy)-1,3,4-thiadiazol-2-amine (627 mg, 44% yield) as a yellow solid. MS (ESI) calc'd for (C₇H₁₁N₃OS) (M+1)⁺, 185.1. found 185.1.

### Step 4 2′-chloro-N-(5-(cyclopentyloxy)-1,3,4-thiadiazol-2-yl)-5′-methoxy-6-methyl-(4,4′-bipyridine)-3-carboxamide

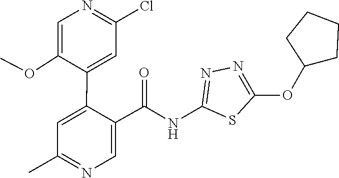 [SMILES: COc1cnc(Cl)cc1-c1cc(C)ncc1C(=O)Nc1nnc(OC2CCCC2)s1]

To a stirred solution of Intermediate G (301 mg, 1.080 mmol) and 5-(cyclopentyloxy)-1,3,4-thiadiazol-2-amine (200 mg, 1.080 mmol) in ACN (2 mL) and N,N-Dimethylformamide (1 mL) were added 1-methylimidazole (0.430 mL, 5.40 mmol) and TCFH (333 mg, 1.188 mmol) at 25° C. The resulting solution was stirred at 25° C. for 1 h. The reaction mixture (3 mL) was purified by prep-HPLC with the following condition: (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 34% B to 50% B in 8 min, 50% B to 95% B in 8.2 min, 95% B to 95% B in 9.5 min, 95% B to 5% B in 11 min, 5% B; Wave Length: 254 nm; RT1 (min): 6; Injection Volume: 0.5 mL; Number Of Runs: 5) to afford 2′-chloro-N-(5-(cyclopentyloxy)-1,3,4-thiadiazol-2-yl)-5′-methoxy-6-methyl-(4,4′-bipyridine)-3-carboxamide (51.7 mg, 10% yield) as a white solid MS (ESI) calc'd for (C₂₀H₂₀ClN₅O₃S) (M+1)⁺ 446.1. found 446.1. ¹H NMR (400 MHz, DMSO-d₆) δ 12.86 (s, 1H), 8.82 (s, 1H), 8.16 (s, 1H), 7.50 (s, 1H), 7.39 (s, 1H), 5.29-5.28 (m, 1H), 3.63 (s, 3H), 2.58-2.51 (m, 3H), 1.99-1.91 (m, 2H), 1.85-1.82 (m, 2H), 1.72-1.59 (m, 4H).

### Example 18

### 2′-chloro-N-(5-(2,2-difluoroethoxy)-1,3,4-thiadiazol-2-yl)-5′-methoxy-6-methyl-(4,4′-bipyridine)-3-carboxamide

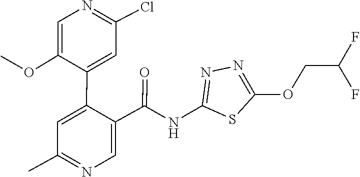 [SMILES: COc1cnc(Cl)cc1-c1cc(C)ncc1C(=O)Nc1nnc(OCC(F)F)s1]

### Step 1 5-(2,2-difluoroethoxy)-1,3,4-thiadiazol-2-amine

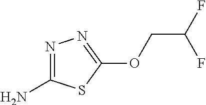 [SMILES: Nc1nnc(OCC(F)F)s1]

To a degassed solution of 2,2-difluoroethane-1-ol (1 g, 12.19 mmol) in dry Tetrahydrofuran (THF) (10 mL) was added sodium hydride (0.975 g, 24.38 mmol, 60%) in portions at 0° C. and stirred for 30 min under nitrogen. Then 5-bromo-1,3,4-thiadiazol-2-amine (2.63 g, 14.63 mmol) was added to the above mixture at 0° C. The resulting solution was stirred at 0˜5° C. for 30 min. The resulting mixture was quenched with water and extracted with ethyl acetate. The combined organic layers were washed with brine, dried over anhydrous sodium sulfate, filtered, and concentrated under vacuum. The resulting residue was dissolved in DMF (4 mL) and was applied to a 80 g C18 column that was eluted with 5˜36% acetonitrile in water within 20 min to afford 5-(2,2-difluoroethoxy)-1,3,4-thiadiazol-2-amine (330 mg, 12% yield) as a white solid. MS (ESI) calculated for (C₄H₅F₂N₃OS) (M+1)⁺, 182.0. found 182.1.

### Step 2 2′-chloro-N-(5-(2,2-difluoroethoxy)-1,3,4-thiadiazol-2-yl)-5′-methoxy-6-methyl-(4,4′-bipyridine)-3-carboxamide

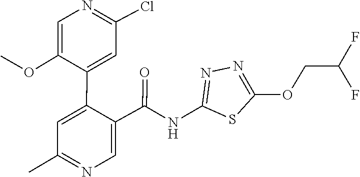 [SMILES: COc1cnc(Cl)cc1-c1cc(C)ncc1C(=O)Nc1nnc(OCC(F)F)s1]

To a solution of Intermediate G (250 mg, 0.897 mmol) in Acetonitrile (3 mL) were sequentially added NMI (221 mg, 2.69 mmol), 5-(2,2-difluoroethoxy)-1,3,4-thiadiazol-2-amine (244 mg, 1.346 mmol) and TCFH (302 mg, 1.076 mmol) at 23° C. and stirred at 30° C. for 2 h. The organic solvent was removed under vacuum. The resulting residue was dissolved in DMF (2 mL) and purified by Combi Flash (Biotage Isolera Prime) which applied to 40 g C18 column that was eluted with 5˜53% acetonitrile in water within 22 min to afford 2′-chloro-N-(5-(2,2-difluoroethoxy)-1,3,4-thiadiazol-2-yl)-5′-methoxy-6-methyl-(4,4′-bipyridine)-3-carboxamide (34.9 mg, 9% yield) as a white solid. MS (ESI) calculated for (C₁₇H₁₄ClF₂N₅O₃S) (M+1)⁺, 442.0. found 442.0. ¹H NMR (400 MHz, DMSO-d₆) δ 13.04 (s, 1H), 8.82 (s, 1H), 8.17 (s, 1H), 7.55 (s, 1H), 7.43 (s, 1H), 6.65-6.25 (m, 1H), 4.85-4.62 (m, 2H), 3.63 (s, 3H), 2.59 (s, 3H).

### Example 19

### 2′-chloro-N-(5-(2-fluoro-2-methylpropoxy)-1,3,4-thiadiazol-2-yl)-5′-methoxy-6-methyl-(4,4′-bipyridine)-3-carboxamide

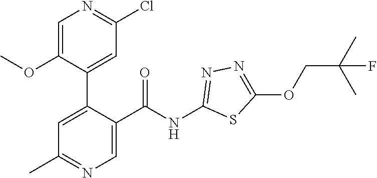 [SMILES: COc1cnc(Cl)cc1-c1cc(C)ncc1C(=O)Nc1nnc(OCC(C)(C)F)s1]

### Step-1: 5-(2-fluoro-2-methylpropoxy)-1,3,4-thiadiazol-2-amine

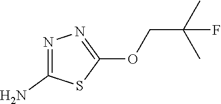

To a stirred solution of 2-fluoro-2-methylpropan-1-ol (600.0 mg, 6.52 mmol) in THF (10 mL) was added NaH (313.0 mg, 7.82 mmol, 60%) in portions at 0° C. and stirred at 0° C. for 30 min under nitrogen atmosphere. Then 5-bromo-1,3,4-thiadiazol-2-amine (1.4 g, 7.82 mmol) was added to the above mixture at 0° C. The resulting solution was stirred at 0° C. for 2 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (1 mL) which was applied to a 40 g C18 column and purified by Combi Flash (Biotage Isolera Prime), eluted with 5˜50% acetonitrile in water within 20 min to afford 5-(2-fluoro-2-methylpropoxy)-1,3,4-thiadiazol-2-amine (150.0 mg, 25%) as a white solid. MS (ESI) calculated for (C₆H₁₀FN₃OS) (M+1)⁺, 192.2. found, 192.1.

### Step-2: 2′-chloro-N-(5-(2-fluoro-2-methylpropoxy)-1,3,4-thiadiazol-2-yl)-5′-methoxy-6-methyl-(4,4′-bipyridine)-3-carboxamide

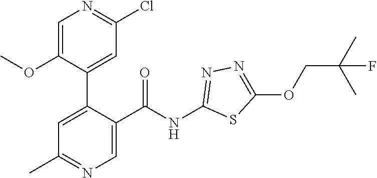 [SMILES: COc1cnc(Cl)cc1-c1cc(C)ncc1C(=O)Nc1nnc(OCC(C)(C)F)s1]

To a stirred solution of 5-(2-fluoro-2-methylpropoxy)-1,3,4-thiadiazol-2-amine (130.0 mg, 0.68 mmol) in acetonitrile (1 mL) were added Intermediate G (189.0 mg, 0.68 mmol) and 1-methylimidazole (278.8 mg, 3.40 mmol). To the above solution was added a solution of TCFH (191.0 mg, 0.68 mmol) in acetonitrile (1 mL) at 23° C. The resulting solution was stirred at 23° C. for 1 hr. The reaction mixture (4 mL) was purified by prep-HPLC with the following condition: (Column: YMC-Actus Triart C18 ExRS, 30*150 mm, 5 um; Mobile Phase A: Water (10 mmol/L NH₄HCO₃), Mobile Phase B: MeOH—Preparative; Flow rate: 60 mL/min; Gradient: 50% B to 70% B in 8 min, 70% B; Wave Length: 254 nm; RT1 (min): 7.2) to afford 2′-chloro-N-(5-(2-fluoro-2-methylpropoxy)-1,3,4-thiadiazol-2-yl)-5′-methoxy-6-methyl-(4,4′-bipyridine)-3-carboxamide (142.0 mg, 46%) as a white solid. MS (ESI) calculated for (C₁₉H₁₉ClFN₅O₃S) (M+1)⁺, 452.1. found, 452.1. ¹H NMR (400 MHz, DMSO-d₆) δ 12.96 (s, 1H), 8.81 (s, 1H), 8.17 (s, 1H), 7.55 (s, 1H), 7.44 (s, 1H), 4.48 (d, J=20.8 Hz, 2H), 3.63 (s, 3H), 2.67 (s, 3H), 1.44 (s, 3H), 1.39 (s, 3H).

### Example 20

### 6′-chloro-N-(5-(2,2-difluoropropoxy)-1,3,4-thiadiazol-2-yl)-3′-methoxy-6-methyl-4,4′-bipyridine-3-carboxamide

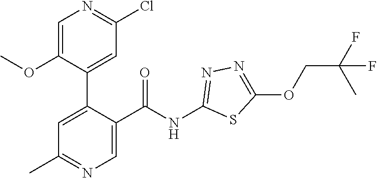 [SMILES: COc1cnc(Cl)cc1-c1cc(C)ncc1C(=O)Nc1nnc(OCC(C)(F)F)s1]

### Step-1: 5-(2,2-difluoropropoxy)-1,3,4-thiadiazol-2-amine

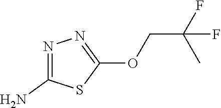

To a degassed solution of 2,2-difluoropropan-1-ol (1.0 g, 10.41 mmol) in dry Tetrahydrofuran (10 mL) was added sodium hydride (0.8 g, 20.82 mmol) in portions at 0° C. and stirred for 30 min. Then 5-bromo-1,3,4-thiadiazol-2-amine (1.8 g, 10.41 mmol) was added to the above mixture at 0° C. The resulting solution was stirred at 0° C. for 30 min. The reaction was quenched with water and extracted with ethyl acetate. The combined organic layers were washed with brine, dried over anhydrous sodium sulfate, filtered, and concentrated under vacuum. The resulting residue was dissolved in DMF (4 mL) and was applied to a 80 g C18 column, purified by Combi Flash (Biotage Isolera Prime) that was eluted with 5˜36% acetonitrile in water within 20 min to afford 5-(2,2-difluoropropoxy)-1,3,4-thiadiazol-2-amine (300.0 mg, 9%) as a brown solid. MS (ESI) calc'd for (C₅H₇F₂N₃OS) (M+1)⁺, 196.0. found 196.0.

### Step-2: 6′-chloro-N-(5-(2,2-difluoropropoxy)-1,3,4-thiadiazol-2-yl)-3′-methoxy-6-methyl-4,4′-bipyridine-3-carboxamide

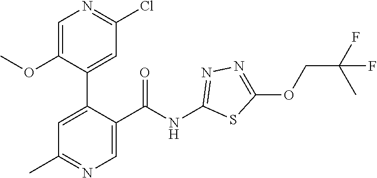 [SMILES: COc1cnc(Cl)cc1-c1cc(C)ncc1C(=O)Nc1nnc(OCC(C)(F)F)s1]

To a solution of Intermediate G (200.0 mg, 0.71 mmol) in Acetonitrile (4 mL) were sequentially added 1-methyl-1H-imidazole (206.0 mg, 2.51 mmol), 5-(2,2-difluoropropoxy)-1,3,4-thiadiazol-2-amine (140.0 mg, 0.71 mmol)) and TCFH (201.0 mg, 0.71 mmol) at 20° C. The mixture was stirred at 30° C. for 16 h. The organic solvent was removed under vacuum. The residue was dissolved in DMF (2 mL) and purified by prep-HPLC with the following condition: (Column: XBridge Shield RP18 OBD Column, 30*150 mm, 5 um; Mobile Phase A: Water (10 mmol/L NH₄HCO₃), Mobile Phase B: MeOH—HPLC; Flow rate: 60 mL/min; Gradient: 44% B to 58% B in 8 min, 58% B to 95% B in 8.2 min, 95% B to 95% B in 9.5 min, 95% B to 2% B in 11 min, 2% B; Wave Length: 254 nm; RT1 (min): 7; Injection Volume: 0.7 mL; Number Of Runs: 3) to afford 2′-chloro-N-(5-(2,2-difluoropropoxy)-1,3,4-thiadiazol-2-yl)-5′-methoxy-6-methyl-(4,4′-bipyridine)-3-carboxamide (24.7 mg, 7%) as a white solid. MS (ESI) calc'd for (C₁₈H₁₆ClF₂N₅O₃S) (M+1)⁺, 456.0. found 456.0. ¹H NMR (400 MHz, DMSO-d₆) δ 13.01 (s, 1H), 8.82 (s, 1H), 8.17 (s, 1H), 7.54 (s, 1H), 7.43 (s, 1H), 4.74 (t, J=12.8 Hz, 2H), 3.63 (s, 3H), 2.59 (s, 3H), 1.74 (t, J=19.2 Hz, 3H).

### Example 21

### 2′-chloro-N-(5-isobutoxy-1,3,4-thiadiazol-2-yl)-5′-methoxy-6-methyl-(4,4′-bipyridine)-3-carboxamide

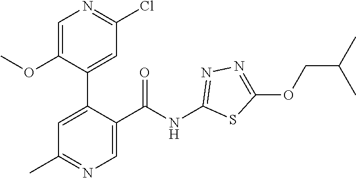 [SMILES: COc1cnc(Cl)cc1-c1cc(C)ncc1C(=O)Nc1nnc(OCC(C)C)s1]

### Step-1: 5-isobutoxy-1,3,4-thiadiazol-2-amine

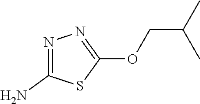

To a degassed solution of 2-methylpropan-1-ol (1.0 g, 13.49 mmol) and in dry Tetrahydrofuran (10 mL) was added NaH (540.0 mg, 13.49 mmol, 60%) in portions at 0° C.

The resulting solution was stirred at 0° C. for 40 min. To the above solution was added 5-bromo-1,3,4-thiadiazol-2-amine (2.4 g, 13.49 mmol) at 0° C. The resulting mixture was then stirred at 0° C. for 1 hr. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which was applied to a 40 g silica gel column that was eluted with 0˜40% ethyl acetate in petroleum ether within 30 min to afford 5-isobutoxy-1,3,4-thiadiazol-2-amine (25.0 mg, 1%) as a white solid. MS (ESI) calc'd for (C₆H₁₁N₃OS) (M+1)⁺, 174.1. found, 174.0.

### Step-2: 2′-chloro-N-(5-isobutoxy-1,3,4-thiadiazol-2-yl)-5′-methoxy-6-methyl-(4,4′-bipyridine)-3-carboxamide

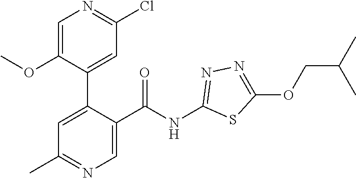 [SMILES: COc1cnc(Cl)cc1-c1cc(C)ncc1C(=O)Nc1nnc(OCC(C)C)s1]

To a mixture of Intermediate G (40.0 mg, 0.14 mmol) in Acetonitrile (3 mL) were added 5-isobutoxy-1,3,4-thiadiazol-2-amine (24.0 mg, 0.14 mmol) and 1-methyl-1H-imidazole (58.0 mg, 0.71 mmol). A solution of TCFH (60.0 mg, 0.21 mmol) in Acetonitrile (1 mL) was added thereto dropwise. The mixture was stirred at 30° C. for 16 hr. The solvents were removed under vacuum. The resulting residue was dissolved in DMF (2 mL) which was applied to a 40 g C18 column and purified by Combi Flash (Biotage Isolera Prime), eluted with 5˜45% acetonitrile in water within 40 min to afford 2′-chloro-N-(5-isobutoxy-1,3,4-thiadiazol-2-yl)-5′-methoxy-6-methyl-(4,4′-bipyridine)-3-carboxamide (21.1 mg, 45%) as a white solid. MS (ESI) calc'd for (C₁₉H₂₀ClN₅O₃S) (M+1)⁺, 434.1. found 434.0. ¹H NMR (400 MHz, DMSO-d₆) δ 12.87 (s, 1H), 8.81 (s, 1H), 8.17 (s, 1H), 7.54 (s, 1H), 7.43 (s, 1H), 4.21 (d, J=6.4 Hz, 2H), 3.64 (s, 3H), 2.59 (s, 3H), 2.15-2.03 (m, J=6.8 Hz, 1H), 0.97 (d, J=6.4 Hz, 6H).

### Example 22 and 23

### (S)-2′-chloro-5′-methoxy-6-methyl-N-(5-((tetrahydrofuran-3-yl)oxy)-1,3,4-thiadiazol-2-yl)-(4,4′-bipyridine)-3-carboxamide and (R)-2′-chloro-5′-methoxy-6-methyl-N-(5-((tetrahydrofuran-3-yl)oxy)-1,3,4-thiadiazol-2-yl)-(4,4′-bipyridine)-3-carboxamide

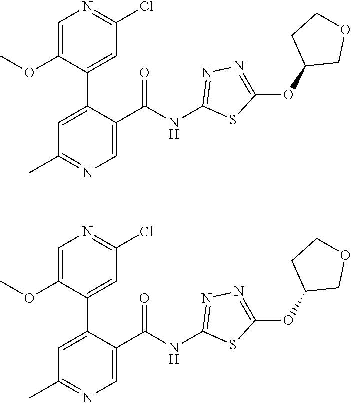 [SMILES: COc1cnc(Cl)cc1-c1cc(C)ncc1C(=O)Nc1nnc(O[C@H]2CCOC2)s1 ; COc1cnc(Cl)cc1-c1cc(C)ncc1C(=O)Nc1nnc(O[C@@H]2CCOC2)s1]

### Step 1 5-((tetrahydrofuran-3-yl) oxy)-1,3,4-thiadiazol-2-amine

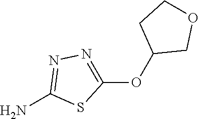 [SMILES: Nc1nnc(OC2CCOC2)s1]

To a degassed solution of tetrahydrofuran-3-ol (1.0 g, 11.35 mmol) in dry Tetrahydrofuran (20 mL) in portions at 0° C. and stirred at 0° C. for 30 min. To the above mixture was added 5-bromo-1,3,4-thiadiazol-2-amine (2.0 g, 11.35 mmol) at 0° C. The resulting solution was stirred at 23° C. for 1 h under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (2.5 mL), applied to a 40 g C18 column and purified by Combi Flash (Biotage Isolera Prime), eluted with 5˜30% acetonitrile in water within 30 min to afford 5-((tetrahydrofuran-3-yl) oxy)-1,3,4-thiadiazol-2-amine (80.0 mg, 3%) as a yellow solid. MS (ESI) calc'd for (C₆H₉N₃O₂S) (M+1)⁺, 188.1. found 188.2.

### Step 2 2′-chloro-5′-methoxy-6-methyl-N-(5-((tetrahydrofuran-3-yl)oxy)-1,3,4-thiadiazol-2-yl)-(4,4′-bipyridine)-3-carboxamide

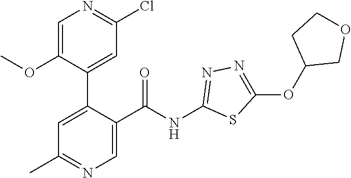 [SMILES: COc1cnc(Cl)cc1-c1cc(C)ncc1C(=O)Nc1nnc(OC2CCOC2)s1]

To a stirred solution of 5-((tetrahydrofuran-3-yl) oxy)-1,3,4-thiadiazol-2-amine (80.0 mg, 0.43 mmol) in N,N-Dimethylformamide (1 mL) and acetonitrile (1 mL) were added Intermediate G (119.0 mg, 0.43 mmol) and 1-methylimidazole (175.0 mg, 2.13 mmol). To the above mixture was added a solution of TCFH (132.0 mg, 0.47 mmol) in acetonitrile (1 mL) at 23° C. The resulting solution was stirred at 23° C. for 1 h. The resulting mixture was dissolved in DMF (3 mL) and was purified by prep-HPLC with the following conditions: (Column: XBridge Shield RP18 OBD Column, 30*150 mm, 5 um; Mobile Phase A: Water (10 mmol/L NH₄HCO₃), Mobile Phase B: ACN; Flow rate: 60 mL/min; Gradient: 17% B to 35% B in 8 min, 35% B to 95% B in 8.2 min, 95% B to 95% B in 9.5 min, 95% B to 5% B in 11 min, 5% B; Wave Length: 254 nm; RT1 (min): 7.3; Injection Volume: 0.5 mL; Number Of Runs: 8) to afford 2′-chloro-5′-methoxy-6-methyl-N-(5-((tetrahydrofuran-3-yl)oxy)-1,3,4-thiadiazol-2-yl)-(4,4′-bipyridine)-3-carboxamide (100.0 mg, 49%) as a white solid. MS (ESI) calc'd for (C₁₉H₁₈ClN₅O₄S) (M+1)⁺, 448.0. found 448.0.

### Step 3 (S)-2′-chloro-5′-methoxy-6-methyl-N-(5-((tetrahydrofuran-3-yl)oxy)-1,3,4-thiadiazol-2-yl)-(4,4′-bipyridine)-3-carboxamide and (R)-2′-chloro-5′-methoxy-6-methyl-N-(5-((tetrahydrofuran-3-yl)oxy)-1,3,4-thiadiazol-2-yl)-(4,4′-bipyridine)-3-carboxamide

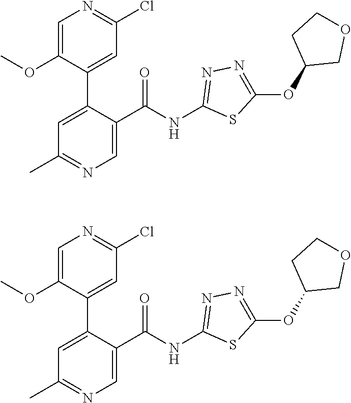 [SMILES: COc1cnc(Cl)cc1-c1cc(C)ncc1C(=O)Nc1nnc(O[C@H]2CCOC2)s1 ; COc1cnc(Cl)cc1-c1cc(C)ncc1C(=O)Nc1nnc(O[C@@H]2CCOC2)s1]

The racemic compound (99.0 mg) was separated by prep-chiral HPLC with the following condition: (Column: CHIRAL ART Cellulose-SC, 2*25 cm, 5 um; Mobile Phase A: Hex—HPLC, Mobile Phase B: MeOH:EtOH=1:1—HPLC; Flow rate: 20 mL/min; Gradient: 50% B to 50% B in 15.5 min; Wave Length: 220/254 nm; RT1 (min): 9.82; RT2 (min): 13.73; Sample Solvent: MeOH:DCM=1:1; Injection Volume: 1 mL; Number Of Runs: 8) to afford (S)-2′-chloro-5′-methoxy-6-methyl-N-(5-((tetrahydrofuran-3-yl)oxy)-1,3,4-thiadiazol-2-yl)-(4,4′-bipyridine)-3-carboxamide (24.3 mg, 24%) as a white solid with retention time at 9.82 minute and (R)-2′-chloro-5′-methoxy-6-methyl-N-(5-((tetrahydrofuran-3-yl)oxy)-1,3,4-thiadiazol-2-yl)-(4,4′-bipyridine)-3-carboxamide (20.9 mg, 21%) as a white solid with retention time at 13.73 minute.

(S)-2′-chloro-5′-methoxy-6-methyl-N-(5-((tetrahydrofuran-3-yl)oxy)-1,3,4-thiadiazol-2-yl)-(4,4′-bipyridine)-3-carboxamide: MS (ESI) calc'd for (C₁₉H₁₈ClN₅O₄S) (M+1)⁺, 448.0. found 448.0. ¹H NMR (400 MHz, DMSO-d₆) δ 12.92 (s, 1H), 8.89 (s, 1H), 8.14 (s, 1H), 7.42 (s, 1H), 7.28 (s, 1H), 5.44 (s, 1H), 3.91-3.81 (m, 3H), 3.81-3.70 (m, 1H), 3.63 (s, 3H), 2.56 (s, 3H), 2.28-2.20 (m, 1H), 2.13-2.05 (m, 1H).

(R)-2′-chloro-5′-methoxy-6-methyl-N-(5-((tetrahydrofuran-3-yl)oxy)-1,3,4-thiadiazol-2-yl)-(4,4′-bipyridine)-3-carboxamide: MS (ESI) calc'd for (C₁₉H₁₈ClN₅O₄S) (M+1)⁺, 448.0. found 448.0. ¹H NMR (400 MHz, DMSO-d₆) δ 12.92 (s, 1H), 8.83 (s, 1H), 8.16 (s, 1H), 7.51 (s, 1H), 7.39 (s, 1H), 5.49 (s, 1H), 3.91-3.81 (m, 3H), 3.81-3.72 (m, 1H), 3.64 (s, 3H), 2.58 (s, 3H), 2.34-2.20 (m, 1H), 2.20-2.11 (m, 1H).

(R)-2′-chloro-5′-methoxy-6-methyl-N-(5-((tetrahydrofuran-3-yl)oxy)-1,3,4-thiadiazol-2-yl)-(4,4′-bipyridine)-3-carboxamide was re-synthesized using the above procedure with (R)-tetrahydrofuran-3-ol as starting material to confirm absolute stereochemistry.

### Example 24

### 2′-chloro-5′-methoxy-6-methyl-N-(5-((3-methyloxetan-3-yl)oxy)-1,3,4-thiadiazol-2-yl)-(4,4′-bipyridine)-3-carboxamide

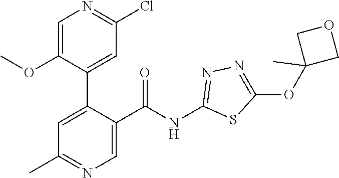 [SMILES: COc1cnc(Cl)cc1-c1cc(C)ncc1C(=O)Nc1nnc(OC2(C)COC2)s1]

### Step-1: 5-((3-methyloxetan-3-yl)oxy)-1,3,4-thiadiazol-2-amine

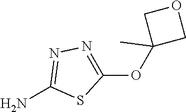

To a degassed solution of 3-methyloxetan-3-ol (500.0 mg, 5.67 mmol) in dry tetrahydrofuran (10 mL) was added sodium hydride (340.0 mg, 8.51 mmol, 60%) in portions at 0° C. and stirred at 0° C. for 1 h under nitrogen atmosphere. Then 5-bromo-1,3,4-thiadiazol-2-amine (1.0 g, 5.67 mmol) was added to the above mixture at 0° C. The mixture was stirred at room temperature for 2 h. The resulting mixture was quenched with water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 40 g silica gel column and eluted with 0˜90% ethyl acetate in petroleum ether within 40 min to afford 5-((3-methyloxetan-3-yl)oxy)-1,3,4-thiadiazol-2-amine (35.0 mg, 1%) as a yellow oil. MS (ESI) calc'd for (C₆H₉N₃O₂S) (M+1)⁺, 188.0. found 188.0.

### Step-2: 2′-chloro-5′-methoxy-6-methyl-N-(5-((3-methyloxetan-3-yl)oxy)-1,3,4-thiadiazol-2-yl)-(4,4′-bipyridine)-3-carboxamide

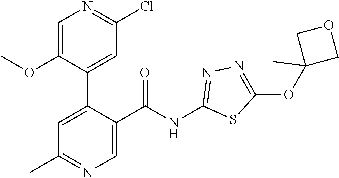 [SMILES: COc1cnc(Cl)cc1-c1cc(C)ncc1C(=O)Nc1nnc(OC2(C)COC2)s1]

To a stirred solution of 5-((3-methyloxetan-3-yl)oxy)-1,3,4-thiadiazol-2-amine (60.0 mg, 0.32 mmol) and Intermediate G (93.0 mg, 0.35 mmol) in acetonitrile (1.5 mL) were sequentially added 1-methylimidazole (131.2 mg, 1.60 mmol) and TCFH (90.0 mg, 0.32 mmol) at 23° C. The resulting solution was stirred at 23° C. for 2 h under nitrogen. The resulting residue was dissolved in acetonitrile (1 mL) which was applied to a 20 g C18 column and purified by Combi Flash (Biotage Isolera Prime), eluted with 544% acetonitrile in water within 25 min and further purified by prep-HPLC with the following conditions: (Column: XBridge Prep OBD C18 Column, 30*150 mm, 5 um; Mobile Phase A: Water (10 mmol/L NH₄HCO₃), Mobile Phase B: ACN; Flow rate: 60 mL/min; Gradient: 20% B to 30% B in 8 min, 30% B; Wave Length: 254 nm; RT1 (min): 7.85) to afford 2′-chloro-5′-methoxy-6-methyl-N-(5-((3-methyloxetan-3-yl)oxy)-1,3,4-thiadiazol-2-yl)-(4,4′-bipyridine)-3-carboxamide (30.0 mg, 20%) as a white solid. MS (ESI) calc'd for (C₁₉H₁₈ClN₅SO₄S) (M+1)⁺, 448.1. found 448.1. ¹H NMR (400 MHz, DMSO-d₆) δ 12.96 (s, 1H), 8.83 (s, 1H), 8.17 (s, 1H), 7.51 (s, 1H), 7.40 (s, 1H), 4.80 (d, J=7.2 Hz, 2H), 4.55 (d, J=7.2 Hz, 2H), 3.64 (s, 3H), 2.59 (s, 3H), 1.80 (s, 3H).

### Example 25

### 2′-chloro-N-(5-((1,1-dioxidotetrahydro-2H-thiopyran-4-yl)oxy)-1,3,4-thiadiazol-2-yl)-5′-methoxy-6-methyl-(4,4′-bipyridine)-3-carboxamide

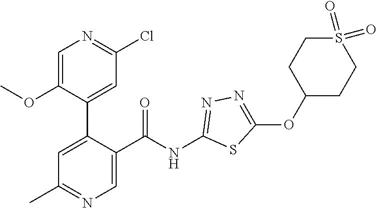 [SMILES: COc1cnc(Cl)cc1-c1cc(C)ncc1C(=O)Nc1nnc(OC2CCS(=O)(=O)CC2)s1]

### Step 1 O-(1,1-dioxidotetrahydro-2H-thiopyran-4-yl)S-methyl Carbonodithioate

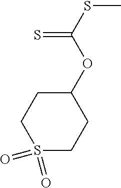

To a degassed solution of 4-hydroxytetrahydro-2H-thiopyran 1,1-dioxide (300.0 mg, 2.00 mmol) in dry Tetrahydrofuran (THF) (6 mL) was added NaH (160.0 mg, 4.00 mmol, 60%) in portions at 0° C. under nitrogen. The resulting solution was stirred at 0° C. for 30 min. To the above solution was added CS₂ (228.0 mg, 3.0 mmol) at 0° C. under nitrogen. The resulting solution was then stirred at 0° C. for 20 min. To the above solution was added MeI (426.0 mg, 3.0 mmol) at 0° C. under nitrogen. The resulting mixture was then stirred at 23° C. for 2 hr. The reaction mixture was quenched by the addition of water and extracted with ethyl acetate.

The combined organic layers were washed with brine, dried over anhydrous sodium sulfate, filtered, and concentrated under vacuum. The resulting residue was dissolved in DCM (1 mL) and purified by Combi Flash (Biotage Isolera Prime) which applied to a 20 g silica gel column that was eluted with 0˜40% ethyl acetate in petroleum ether within 30 min to afford O-(1,1-dioxidotetrahydro-2H-thiopyran-4-yl)S-methyl carbonodithioate (370.0 mg, 76%) as a white solid MS (ESI) calculated for (C₇H₁₂O₃S₃) (M+1)⁺, 241.0. found, 241.0.

### Step 2 O-(1,1-dioxidotetrahydro-2H-thiopyran-4-yl) hydrazinecarbothioate

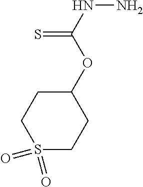 [SMILES: NNC(=S)OC1CCS(=O)(=O)CC1]

To a stirred solution of O-(1,1-dioxidotetrahydro-2H-thiopyran-4-yl)S-methyl carbonodithioate (350.0 mg, 1.46 mmol) in Methanol (5 mL) was added Hydrazinium hydroxide solution (58.0 mg, 1.46 mmol) at 23° C. The resulting solution was stirred at 23° C. for 1 h. The resulting mixture was concentrated under vacuum to afford O-(1,1-dioxidotetrahydro-2H-thiopyran-4-yl) hydrazinecarbothioate (322.0 mg, crude) as a yellow solid. MS (ESI) calculated for (C₆H₁₂N₂O₃S₂) (M+1)⁺, 225.0. found, 225.0.

### Step 3 4-((5-amino-1,3,4-thiadiazol-2-yl)oxy)tetrahydro-2H-thiopyran 1,1-dioxide

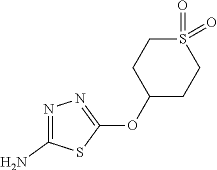 [SMILES: Nc1nnc(OC2CCS(=O)(=O)CC2)s1]

To a stirred solution of O-(1,1-dioxidotetrahydro-2H-thiopyran-4-yl) hydrazinecarbothioate (322.0 mg, 1.44 mmol) in Methanol (3 mL) were sequentially added TEA (436.3 mg, 4.32 mmol) and cyanic bromide (167.0 mg, 1.58 mmol) at 0° C. The resulting solution was stirred at 23° C. for 2 hours. The reaction mixture was diluted with ice cold water and extracted with ethyl acetate. The combined organic layers were washed with brine, dried over anhydrous sodium sulfate, filtered, and concentrated under vacuum afford 4-((5-amino-1,3,4-thiadiazol-2-yl)oxy)tetrahydro-2H-thiopyran 1,1-dioxide (230.0 mg, 46%) as an yellow solid MS (ESI) calculated for (C₇H₁₁N₃O₃S₂) (M+1)⁺, 250.0. found, 250.0.

### Step 4 2′-chloro-N-(5-((1,1-dioxidotetrahydro-2H-thiopyran-4-yl)oxy)-1,3,4-thiadiazol-2-yl)-5′-methoxy-6-methyl-(4,4′-bipyridine)-3-carboxamide

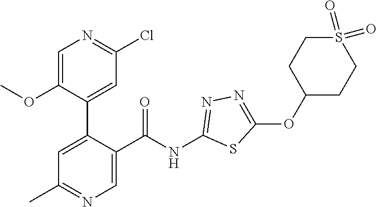 [SMILES: COc1cnc(Cl)cc1-c1cc(C)ncc1C(=O)Nc1nnc(OC2CCS(=O)(=O)CC2)s1]

To a stirred solution of 4-((5-amino-1,3,4-thiadiazol-2-yl)oxy)tetrahydro-2H-thiopyran 1,1-dioxide (190.0 mg, 0.76 mmol) and Intermediate G (234.0 mg, 0.84 mmol) in acetonitrile (0.5 mL) were sequentially added 1-methylimidazole (0.3 mL) and TCFH (214.0 mg, 0.76 mmol) in acetonitrile (0.2 mL) at 23° C. under nitrogen. The resulting solution was stirred at 23° C. for 2 hours. The mixture was dissolved in acetonitrile (2 mL) which was applied to a 20 g C18 column and purified by Combi Flash (Biotage Isolera Prime), eluted with 5˜40% acetonitrile in water within 30 min to afford a off-white solid (88% purity). The product was dissolved in DMF (3 mL) and was further purified by prep-HPLC with the following condition: (Column: Xselect CSH F-Phenyl OBD column, 19*250 mm, 5 um; Mobile Phase A: Water (0.05% TFA), Mobile Phase B: MeOH—HPLC; Flow rate: 25 mL/min; Gradient: 53% B to 68% B in 10 min, 68% B to 95% B in 10.2 min, 95% B to 95% B in 12 min, 95% B to 5% B in 12.2 min, 5% B to 5% B in 14 min; Wave Length: 254 nm; RT1 (min): 8; Injection Volume: 0.7 mL; Number Of Runs: 4) to afford 2′-chloro-N-(5-((1,1-dioxidotetrahydro-2H-thiopyran-4-yl)oxy)-1,3,4-thiadiazol-2-yl)-5′-methoxy-6-methyl-(4,4′-bipyridine)-3-carboxamide (100.0 mg, 25%) as a white solid. MS (ESI) calculated for (C₂₀H₂₀ClN₅O₅S₂) (M+1)⁺, 510.1. found, 510.2. ¹H NMR (400 MHz, DMSO-d₆) δ 8.84 (s, 1H), 8.15 (s, 1H), 7.65-7.48 (m, 2H), 5.25-5.15 (m, 1H), 3.62 (s, 3H), 3.29-3.10 (m, 4H), 2.63 (s, 3H), 2.44-2.26 (m, 4H).

### Example 26 and 27

### 2′-chloro-N-(5-(((1s,4s)-4-hydroxycyclohexyl)oxy)-1,3,4-thiadiazol-2-yl)-5′-methoxy-6-methyl-[4,4′-bipyridine]-3-carboxamide and 2′-chloro-N-(5-(((1r,4r)-4-hydroxycyclohexyl)oxy)-1,3,4-thiadiazol-2-yl)-5′-methoxy-6-methyl-[4,4′-bipyridine]-3-carboxamide

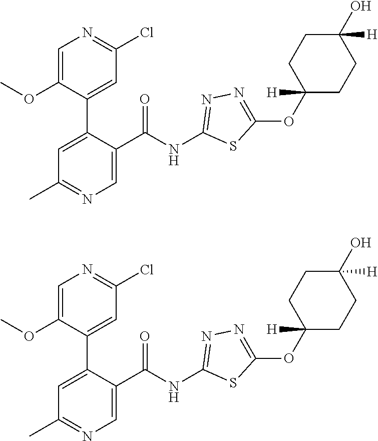 [SMILES: COc1cnc(Cl)cc1-c1cc(C)ncc1C(=O)Nc1nnc(O[C@H]2CC[C@@H](O)CC2)s1 ; COc1cnc(Cl)cc1-c1cc(C)ncc1C(=O)Nc1nnc(O[C@H]2CC[C@H](O)CC2)s1]

### Step 1 O-(4-((tert-butyldimethylsilyl)oxy)cyclohexyl)S-methyl Carbonodithioate

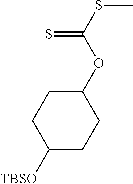 [SMILES: CSC(=S)OC1CCC(O[Si](C)(C)C(C)(C)C)CC1]

To a degassed solution of 4-((tert-butyldimethylsilyl)oxy)cyclohexan-1-ol (550.0 mg, 2.39 mmol) in dry tetrahydrofuran (4 mL) was added NaH (191.0 mg, 4.77 mmol, 60%) at 0° C. and stirred at 0° C. for 30 min under nitrogen atmosphere. Then CS₂ (0.2 mL, 3.58 mmol) was added to the above mixture at 0° C. and stirred at 0° C. for 20 min. To the above solution was added MeI (0.2 mL, 3.58 mmol) at 0° C. under nitrogen. The resulting mixture was then stirred at 0° C. for 1 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (1 mL) and purified by Combi Flash (Biotage Isolera Prime) which applied to a 40 g silica gel column that was eluted with 0˜20% ethyl acetate in petroleum ether within 20 min to afford O-(4-((tert-butyldimethylsilyl)oxy)cyclohexyl)S-methyl carbonodithioate (711.0 mg, 74% yield) as a yellow oil.

### Step 2 O-(4-((tert-butyldimethylsilyl)oxy)cyclohexyl) hydrazinecarbothioate

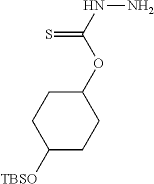 [SMILES: CC(C)(C)[Si](C)(C)OC1CCC(OC(=S)NN)CC1]

To a stirred solution of O-(4-((tert-butyldimethylsilyl)oxy)cyclohexyl)S-methyl carbonodithioate (710.0 mg, 2.22 mmol) in methanol (4 mL) was added hydrazine (0.1 mL, 2.22 mmol) at 20° C. The resulting solution was stirred at 20° C. for 30 min. The volatiles were removed under vacuum to afford O-(4-((tert-butyldimethylsilyl)oxy)cyclohexyl) hydrazinecarbothioate (670.0 mg, crude) as a light yellow oil. MS (ESI) calculated for (C₁₃H₂₈N₂O₂SSi) (M+1)⁺, 305.0. found, 305.3.

### Step 3 5-((4-((tert-butyldimethylsilyl)oxy)cyclohexyl)oxy)-1,3,4-thiadiazol-2-amine

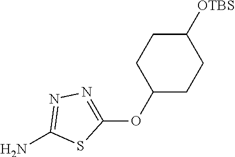 [SMILES: CC(C)(C)[Si](C)(C)OC1CCC(Oc2nnc(N)s2)CC1]

To a stirred solution of O-(4-((tert-butyldimethylsilyl)oxy)cyclohexyl) hydrazinecarbothioate (730.0 mg, 2.40 mmol) in methanol (3 mL) were sequentially added TEA (0.7 mL, 4.79 mmol) and BrCN (279.0 mg, 2.64 mmol) at 20° C. The resulting solution was stirred at 20° C. for 1 hr. The resulting residue was dissolved in DMF (3 mL) which was applied to a 40.0 g C18 column and purified by Combi Flash (Biotage Isolera Prime), eluted with 5˜50% acetonitrile in water within 40 min to afford 5-((4-((tert-butyldimethylsilyl)oxy)cyclohexyl)oxy)-1,3,4-thiadiazol-2-amine (290.0 mg, 33%) as a white solid. MS (ESI) calculated for (C₁₄H₂₇N₃O₂SSi) (M+1)⁺, 330.0. found, 659.5.

### Step 4 N-(5-((4-((tert-butyldimethylsilyl)oxy)cyclohexyl)oxy)-1,3,4-thiadiazol-2-yl)-2′-chloro-5′-methoxy-6-methyl-(4,4′-bipyridine)-3-carboxamide

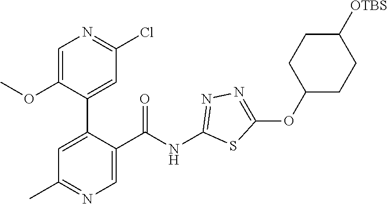 [SMILES: COc1cnc(Cl)cc1-c1cc(C)ncc1C(=O)Nc1nnc(OC2CCC(O[Si](C)(C)C(C)(C)C)CC2)s1]

To a stirred solution of 5-((4-((tert-butyldimethylsilyl)oxy)cyclohexyl)oxy)-1,3,4-thiadiazol-2-amine (290.0 mg, 0.88 mmol) in acetonitrile (5 mL) were sequentially added Intermediate

G (270.0 mg, 0.97 mmol), 1-Methylimidazole (361.0 mg, 4.40 mmol). Then a solution of TCFH (247.0 mg, 0.88 mmol) in acetonitrile (1 mL) was added to the above mixture at 20° C. The resulting solution was stirred at 20° C. for 16 hr. The volatiles were removed under vacuum. The resulting residue was dissolved in DMF (4 mL) which was applied to a 40.0 g C18 column and purified by Combi Flash (Biotage Isolera Prime), eluted with 5˜60% acetonitrile in water within 40 min to afford N-(5-((4-((tert-butyldimethylsilyl)oxy)cyclohexyl)oxy)-1,3,4-thiadiazol-2-yl)-2′-chloro-5′-methoxy-6-methyl-(4,4′-bipyridine)-3-carboxamide (400.0 mg, 75%) as a white solid. MS (ESI) calculated for (C₂₇H₃₆ClN₅O₄SSi) (M+1)⁺, 590.0. found, 590.4.

### Step 5 2′-chloro-N-(5-(((1s,4s)-4-hydroxycyclohexyl)oxy)-1,3,4-thiadiazol-2-yl)-5′-methoxy-6-methyl-[4,4′-bipyridine]-3-carboxamide, and 2′-chloro-N-(5-(((1r,4r)-4-hydroxycyclohexyl)oxy)-1,3,4-thiadiazol-2-yl)-5′-methoxy-6-methyl-[4,4′-bipyridine]-3-carboxamide

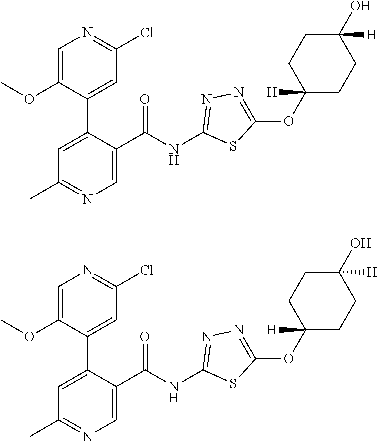 [SMILES: COc1cnc(Cl)cc1-c1cc(C)ncc1C(=O)Nc1nnc(O[C@H]2CC[C@@H](O)CC2)s1 ; COc1cnc(Cl)cc1-c1cc(C)ncc1C(=O)Nc1nnc(O[C@H]2CC[C@H](O)CC2)s1]

To a mixture of N-(5-((4-((tert-butyldimethylsilyl)oxy)cyclohexyl)oxy)-1,3,4-thiadiazol-2-yl)-2′-chloro-5′-methoxy-6-methyl-(4,4′-bipyridine)-3-carboxamide (400.0 mg, 0.68 mmol) in dichloromethane (2 mL) was added trifluoroacetic acid (0.4 mL). The mixture was stirred at 20° C. for 30 min. The organic solvent was removed under vacuum. The residue was diluted with water. The aqueous layer was basified with NaHCO₃ solution and extracted with ethyl acetate. The combined organic layers were washed with brine, dried over anhydrous sodium sulfate, filtered, and concentrated under vacuum. The resulting residue was dissolved in DMF (2 mL) which was applied to a 40.0 g C18 column and purified by Combi Flash (Biotage Isolera Prime), eluted with 25˜90% acetonitrile in water within 45 min to afford 2′-chloro-N-(5-((4-hydroxycyclohexyl)oxy)-1,3,4-thiadiazol-2-yl)-5′-methoxy-6-methyl-(4,4′-bipyridine)-3-carboxamide (204.0 mg, 63%) as a white solid. The mixture of compounds (204.0 mg) was separated by prep-chiral HPLC with the following condition: (Column: CHIRALPAK IE, 2*25 cm, 5 um; Mobile Phase A: Hex—HPLC, Mobile Phase B: MeOH:EtOH=1:1—HPLC; Flow rate: 14 mL/min; Gradient: 80% B to 80% B in 29 min; Wave Length: 220/254 nm; RT1 (min): 16.46; RT2 (min): 20.27; Sample Solvent: MeOH:DCM=1:1; Injection Volume: 0.5 mL; Number Of Runs: 8) to afford 2′-chloro-N-(5-(((1s,4s)-4-hydroxycyclohexyl)oxy)-1,3,4-thiadiazol-2-yl)-5′-methoxy-6-methyl-[4,4′-bipyridine]-3-carboxamide, (45.6 mg, 22%) as a white solid with the first peak on chiral HPLC and 2′-chloro-N-(5-(((1r,4r)-4-hydroxycyclohexyl)oxy)-1,3,4-thiadiazol-2-yl)-5′-methoxy-6-methyl-[4,4′-bipyridine]-3-carboxamide (98.5 mg, 48%) as a white solid with the second peak on chiral HPLC. The absolute stereochemistry was not determined and was arbitrarily assigned.

2′-chloro-N-(5-(((1s,4s)-4-hydroxycyclohexyl)oxy)-1,3,4-thiadiazol-2-yl)-5′-methoxy-6-methyl-[4,4′-bipyridine]-3-carboxamide: MS (ESI) calculated for (C₂₁H₂₂ClN₅O₄S) (M+1)⁺, 476.0. found, 476.2, ¹H NMR (400 MHz, DMSO-d₆) δ 12.89 (s, 1H), 8.81 (s, 1H), 8.17 (s, 1H), 7.52 (s, 1H), 7.41 (s, 1H), 4.93-4.90 (m, 1H), 4.55-4.54 (m, 1H), 3.63-3.59 (m, 4H), 2.58 (s, 3H), 1.99-1.91 (m, 4H), 1.75-1.72 (m, 4H).

2′-chloro-N-(5-(((1r,4r)-4-hydroxycyclohexyl)oxy)-1,3,4-thiadiazol-2-yl)-5′-methoxy-6-methyl-[4,4′-bipyridine]-3-carboxamide: MS (ESI) calculated for (C₂₁H₂₂ClN₅O₄S) (M+1)⁺, 476.0. found, 476.2, ¹H NMR (400 MHz, DMSO-d₆) δ 12.87 (s, 1H), 8.81 (s, 1H), 8.16 (s, 1H), 7.52 (s, 1H), 7.43 (s, 1H), 4.89-4.82 (m, 1H), 4.61-4.60 (m, 1H), 3.63 (s, 3H), 3.62-3.45 (m, 1H), 2.59 (s, 3H), 2.14-2.08 (m, 2H), 1.86-1.82 (m, 2H), 1.60-1.50 (m, 2H), 1.38-1.24 (m, 2H).

### Example 28

### 2′-chloro-N-(5-ethoxy-1,3,4-thiadiazol-2-yl)-5′-methoxy-6-methyl-(4,4′-bipyridine)-3-carboxamide

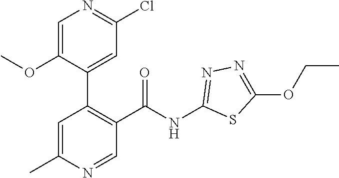 [SMILES: CCOc1nnc(NC(=O)c2cnc(C)cc2-c2cc(Cl)ncc2OC)s1]

### Step-1: O-ethyl Hydrazinecarbothioate

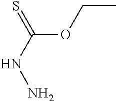

A mixture of potassium O-ethyl carbonodithioate (2.0 g, 12.48 mmol) and Hydrazine hydrate (0.8 g, 12.48 mmol) in Methanol (20 mL) were stirred at 20° C. for 2 hr. The reaction mixture was quenched by the addition of water and extracted with ethyl acetate. The combined organic layers were washed with brine, dried over anhydrous sodium sulfate, filtered, and concentrated under vacuum to afford O-ethyl hydrazinecarbothioate (1.4 g, crude) as a yellow oil. MS (ESI) calc'd for (C₃H₈N₂OS) (M+1)⁺, 121.0. found 121.0.

### Step-2: 5-ethoxy-1,3,4-thiadiazol-2-amine

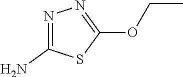

To a mixture of O-ethyl hydrazinecarbothioate (1.4 g, 9.32 mmol) in Methanol (10 mL) were added NaOH (0.8 g, 18.64 mmol) and cyanic bromide (1.1 g, 10.25 mmol) at 20° C. The resulting solution was stirred at 20° C. for 30 min. The reaction mixture was quenched by the addition of water and extracted with ethyl acetate. The combined organic layers were washed with brine, dried over anhydrous sodium sulfate, filtered, and concentrated under vacuum. The residue was dissolved in DMF (3 mL) and purified by Combi Flash (Biotage Isolera Prime) which applied to a 40 g C18 column, eluted with 5˜40% acetonitrile in water within 45 min to afford 5-ethoxy-1,3,4-thiadiazol-2-amine (280 mg, 10%) as a red solid. MS (ESI) calc'd for (C₄H₇N₃OS) (M+1)⁺, 146.0. found 146.0.

### Step-3: 2′-chloro-N-(5-ethoxy-1,3,4-thiadiazol-2-yl)-5′-methoxy-6-methyl-(4,4′-bipyridine)-3-carboxamide

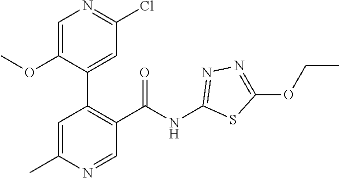 [SMILES: CCOc1nnc(NC(=O)c2cnc(C)cc2-c2cc(Cl)ncc2OC)s1]

To a mixture of 5-ethoxy-1,3,4-thiadiazol-2-amine (268.0 mg, 0.92 mmol) in acetonitrile (2 mL) were added Intermediate G (260.0 mg, 0.84 mmol), and NMI (344.0 mg, 4.20 mmol). A solution of TCFH (259.0 mg, 0.92 mmol) in acetonitrile (1 mL) was added thereto dropwise under nitrogen. The mixture was stirred at 20° C. for 2 hr under nitrogen. The resulting mixture was dissolved in DMF (6 mL) and was purified by Prep-HPLC with the following condition: (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 25% B to 40% B in 8 min, 40% B to 95% B in 8.2 min, 95% B to 95% B in 9.5 min, 95% B to 5% B in 11 min, 5% B; Wave Length: 254 nm; RT1 (min): 7.5; Injection Volume: 1 mL; Number Of Runs: 6) to afford 2′-chloro-N-(5-ethoxy-1,3,4-thiadiazol-2-yl)-5′-methoxy-6-methyl-(4,4′-bipyridine)-3-carboxamide (144.3 mg, 42%) as a white solid. MS (ESI) calc'd for (C₁₇H₁₆ClN₅O₃S) (M+1)⁺, 406.0. found 406.0. ¹H NMR (400 MHz, DMSO-d₆) δ 12.81 (s, 1H), 8.83 (s, 1H), 8.16 (s, 1H), 7.51 (s, 1H), 7.39 (s, 1H), 4.48-4.42 (m, 2H), 3.63 (s, 3H), 2.58 (s, 3H), 1.37 (t, J=8.0 Hz, 3H).

### Example 29

### 2′-chloro-5′-methoxy-6-methyl-N-(5-(2,2,2-trifluoroethoxy)-1,3,4-thiadiazol-2-yl)-(4,4′-bipyridine)-3-carboxamide

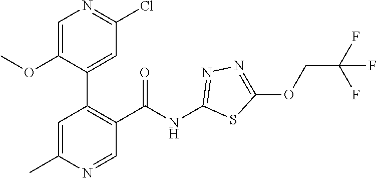 [SMILES: COc1cnc(Cl)cc1-c1cc(C)ncc1C(=O)Nc1nnc(OCC(F)(F)F)s1]

### Step-1: 5-(2,2,2-trifluoroethoxy)-1,3,4-thiadiazol-2-amine

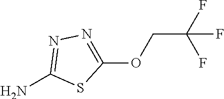 [SMILES: Nc1nnc(OCC(F)(F)F)s1]

To a solution of 2,2,2-trifluoroethan-1-ol (1.0 g, 10.00 mmol) in Tetrahydrofuran (30 mL) was added NaH (0.80 g, 19.99 mmol, 60%) in portions at 0° C. and stirred at 0° C. for 1 hr. To the above solution was added 5-bromo-1,3,4-thiadiazol-2-amine (1.8 g, 10.00 mmol) at 0° C. The resulting solution was stirred at 0° C. for 2 hr. The reaction mixture was quenched by the addition of water and extracted with ethyl acetate. The combined organic layers were washed with brine, dried over anhydrous sodium sulfate, filtered, and concentrated under vacuum to afford the crude (1.3 g, crude) as a grey solid. The resulting residue was dissolved in DMF (8 mL) which was applied to a 80 g C18 column and purified by Combi Flash (Biotage Isolera Prime), eluted with 5˜23% acetonitrile in water within 39 min to afford 5-(2,2,2-trifluoroethoxy)-1,3,4-thiadiazol-2-amine (583.0 mg, 25%) as a grey solid. MS (ESI) calc'd for (C₄H₄F₃N₃OS) (M+1)⁺, 200.0. found 200.0.

### Step-2: 2′-chloro-5′-methoxy-6-methyl-N-(5-(2,2,2-trifluoroethoxy)-1,3,4-thiadiazol-2-yl)-(4,4′-bipyridine)-3-carboxamide

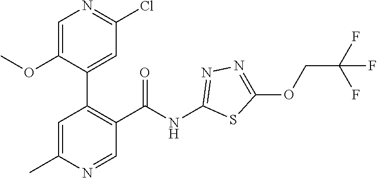 [SMILES: COc1cnc(Cl)cc1-c1cc(C)ncc1C(=O)Nc1nnc(OCC(F)(F)F)s1]

To a mixture of Intermediate G (100.0 mg, 0.35 mmol) in Acetonitrile (2 mL) were added 5-(2,2,2-trifluoroethoxy)-1,3,4-thiadiazol-2-amine (100.0 mg, 0.423 mmol) and 1-methyl-1H-imidazole (87.0 mg, 1.05 mmol). A solution of TCFH (148.0 mg, 0.52 mmol) in Acetonitrile (1 mL) was added thereto dropwise under nitrogen. The resulting solution was stirred at 20° C. for 2 hr under nitrogen. The resulting mixture was purified by Combi Flash (Biotage Isolera Prime) which applied to a 40 g C18 column that was eluted with 5˜54% acetonitrile in water within 45 min to afford 2′-chloro-5′-methoxy-6-methyl-N-(5-(2,2,2-trifluoroethoxy)-1,3,4-thiadiazol-2-yl)-(4,4′-bipyridine)-3-carboxamide (60.7 mg, 36%) as a white solid. MS (ESI) calc'd for (C₁₇H₁₃ClF₃N₅O₃S) (M+1)⁺, 460.0. found 460.0. ¹H NMR (400 MHz, DMSO-d₆) δ 13.08 (s, 1H), 8.82 (s, 1H), 8.17 (s, 1H), 7.55 (s, 1H), 7.44 (s, 1H), 5.16-5.22 (m, 2H), 3.63 (s, 3H), 2.60 (s, 3H).

### Example 30

### N-(5-methoxy-1,3,4-thiadiazol-2-yl)-4-(2-methoxy-5-methylphenyl)-6-methylnicotinamide

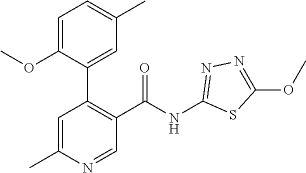

### Step-1: methyl 4-(2-methoxy-5-methylphenyl)-6-methylpyridine-3-carboxylate

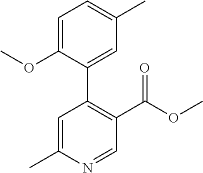

A degassed mixture of methyl 4-chloro-6-methylpyridine-3-carboxylate (500.0 mg, 2.69 mmol), 2-methoxy-5-methylphenylboronic acid (894.2 mg, 5.388 mmol), Pd(dppf)Cl₂ (394.21 mg, 0.53 mmol), Potassium carbonate (744.6 mg, 5.38 mmol) in dioxane (10.0 mL) and Water (1.0 mL) was stirred at 110° C. for 2 h under nitrogen. The solvent was removed under vacuum. The residue was purified by flash column chromatography with 0˜44% ethyl acetate in petroleum ether to afford methyl 4-(2-methoxy-5-methylphenyl)-6-methylpyridine-3-carboxylate (800.0 mg, 57%) as a yellow oil. MS (ESI) calc'd for (C16H17NO3) (M+1)+, 272.1. found 272.1.

### Step-2: 4-(2-methoxy-5-methylphenyl)-6-methylpyridine-3-carboxylic Acid

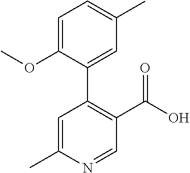 [SMILES: COc1ccc(C)cc1-c1cc(C)ncc1C(=O)O]

A mixture of methyl 4-(2-methoxy-5-methylphenyl)-6-methylpyridine-3-carboxylate (700.0 mg, 2.58 mmol), NaOH (309.5 mg, 7.74 mmol) in THF (10.0 mL) and Water (3.0 mL) was stirred at room temperature for 16 h. The reaction mixture was acidified with HCl (1 N) to pH ˜4. The aqueous layer was extracted with ethyl acetate. The combined organic solution was dried over sodium sulfate, filtered, and concentrated under vacuum to afford 4-(2-methoxy-5-methylphenyl)-6-methylpyridine-3-carboxylic acid (550.0 mg, crude) as a yellow oil. MS (ESI) calc'd for (C15H15NO3) (M+1)+, 258.1. found 258.4.

### Step-3: N-(5-methoxy-1,3,4-thiadiazol-2-yl)-4-(2-methoxy-5-methylphenyl)-6-methylnicotinamide

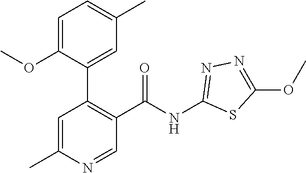

To a stirred solution of 4-(2-methoxy-5-methylphenyl)-6-methylnicotinic acid (100.0 mg, 0.39 mmol) in Acetonitrile (1 mL) were added 5-methoxy-1,3,4-thiadiazol-2-amine (51.1 mg, 0.39 mmol) and 1-methylimidazole (160.0 mg, 1.94 mmol) at 23° C. To the above solution was added a solution of TCFH (109.6 mg, 0.39 mmol) in Acetonitrile (1 mL) at 23° C. under nitrogen. The resulting mixture was then stirred at 23° C. for 2 hr under nitrogen. The mixture was diluted with DMF (2 mL) and was purified by prep-HPLC with the following conditions: (Column: XBridge Shield RP18 OBD Column, 30*150 mm, 5 μm; Mobile Phase A: Water (10 mmol/L NH4HCO3), Mobile Phase B: ACN; Flow rate: 60 mL/min; Gradient: 27% B to 43% B in 8 min, 43% B to 95% B in 8.2 min, 95% B to 95% B in 9.5 min, 95% B to 5% B in 11 min, 5% B; Wave Length: 254 nm; RT1 (min): 7.4; Injection Volume: 0.5 mL; Number Of Runs: 5) to afford N-(5-methoxy-1,3,4-thiadiazol-2-yl)-4-(2-methoxy-5-methylphenyl)-6-methylnicotinamide (72.6 mg, 50%) as a white solid. MS (ESI) calc'd for (C18H18N4O3S) (M+1)+, 371.1. found, 371.1. 1H NMR (400 MHz, DMSO-d6) δ 12.69 (s, 1H), 8.65 (s, 1H), 7.29 (s, 1H), 7.26-7.15 (m, 2H), 6.86 (d, J=8.4 Hz, 1H), 4.06 (s, 3H), 3.46 (s, 3H), 2.56 (s, 3H), 2.31 (s, 3H).

### Example 31

### 2′-chloro-N-(5-hydroxy-1,3,4-thiadiazol-2-yl)-5′-methoxy-6-methyl-(4,4′-bipyridine)-3-carboxamide

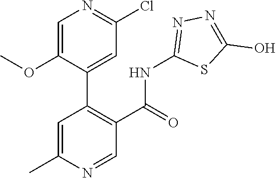 [SMILES: COc1cnc(Cl)cc1-c1cc(C)ncc1C(=O)Nc1nnc(O)s1]

### Step-1: 2′-chloro-N-(5-((5-chloropyridin-2-yl)methoxy)-1,3,4-thiadiazol-2-yl)-5methoxy-6-methyl-(4,4′-bipyridine)-3-carboxamide

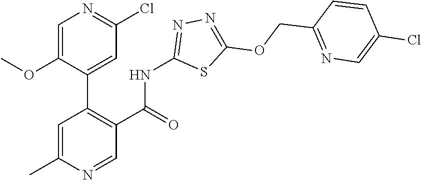 [SMILES: COc1cnc(Cl)cc1-c1cc(C)ncc1C(=O)Nc1nnc(OCc2ccc(Cl)cn2)s1]

To a solution of Intermediate G (300.0 mg, 1.08 mmol) in Acetonitrile (2 mL) were added Intermediate B (261.1 mg, 1.08 mmol) and 1-methylimidazole (441.2 mg, 5.38 mmol) at 20° C. under nitrogen. To the above solution was added a solution of TCFH (301.5 mg, 1.08 mmol) in Acetonitrile (2 mL) at 20° C. under nitrogen. The resulting mixture was then stirred at 20° C. for 1 hr. The mixture was dissolved in DMF (1 mL) which was applied to a 20 g C18 column and purified by Combi Flash (Biotage Isolera Prime), eluted with 5˜55% acetonitrile in water within 30 min to afford 2′-chloro-N-(5-((5-chloropyridin-2-yl)methoxy)-1,3,4-thiadiazol-2-yl)-5′-methoxy-6-methyl-(4,4′-bipyridine)-3-carboxamide (372.0 mg, 63%) as a white solid. MS (ESI) calc'd for (C₂₁H₁₆Cl₂N₆O₃S) (M+1)⁺, 503.1. found, 503.1.

### Step-2: 2′-chloro-N-(5-hydroxy-1,3,4-thiadiazol-2-yl)-5′-methoxy-6-methyl-(4,4′-bipyridine)-3-carboxamide

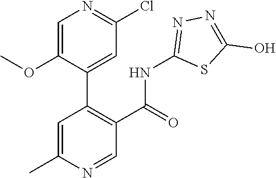 [SMILES: COc1cnc(Cl)cc1-c1cc(C)ncc1C(=O)Nc1nnc(O)s1]

A solution of 2′-chloro-N-(5-((5-chloropyridin-2-yl)methoxy)-1,3,4-thiadiazol-2-yl)-5′-methoxy-6-methyl-(4,4′-bipyridine)-3-carboxamide (320.0 mg, 0.64 mmol) in concentrated hydrochloric acid (1 mL) was stirred at 20° C. for 1 hr. The mixture was diluted with water. The aqueous layer was neutralized with NaHCO₃ solution and extracted with ethyl acetate. The combined organic layers were washed with brine, dried over anhydrous sodium sulfate, filtered, and concentrated under vacuum. The resulting residue was dissolved in DMF (1 mL) which was applied to a 20 g C18 column and purified by Combi Flash (Biotage Isolera Prime), eluted with 5˜60% acetonitrile in water within 25 min to afford 2′-chloro-N-(5-hydroxy-1,3,4-thiadiazol-2-yl)-5′-methoxy-6-methyl-(4,4′-bipyridine)-3-carboxamide (61.4 mg, 25% yield) as a white solid. MS (ESI) calc'd for (C₁₅H₁₂ClN₅O₃S) (M+1)⁺, 378.1. found 378.1. ¹H NMR (400 MHz, DMSO-d₆) δ 12.35-12.30 (m, 2H), 8.75 (s, 1H), 8.20 (s, 1H), 7.52 (s, 1H), 7.47 (s, 1H), 3.72 (s, 3H), 2.58 (s, 3H).

### Example 32

### 4-(2-(difluoromethoxy)-6-fluorophenyl)-N-(5-methoxy-1,3,4-thiadiazol-2-yl)-6-methylnicotinamide

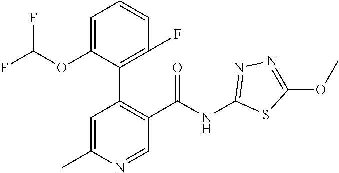 [SMILES: COc1nnc(NC(=O)c2cnc(C)cc2-c2c(F)cccc2OC(F)F)s1]

A mixture of Intermediate D (65.0 mg, 0.19 mmol) in Acetonitrile (1 mL) were added 5-methoxy-1,3,4-thiadiazol-2-amine (28.4 mg, 0.21 mmol) and NMI (81.0 mg, 0.98 mmol). A solution of TCFH (60.6 mg, 0.21 mmol) in Acetonitrile (0.5 mL) was added thereto dropwise under nitrogen. The mixture was stirred at 20° C. for 2 hr under nitrogen. The resulting mixture was dissolved in DMF (2 mL) and was purified by prep-HPLC with the following condition: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 38% B in 8 min, 38% B to 95% B in 8.2 min, 95% B to 95% B in 9.5 min, 95% B to 5% B in 11 min, 5% B; Wave Length: 254 nm; RT1 (min): 7.67; Injection Volume: 0.7 mL; Number Of Runs: 3) to afford 4-(2-(difluoromethoxy)-6-fluorophenyl)-N-(5-methoxy-1,3,4-thiadiazol-2-yl)-6-methylnicotinamide (54.4 mg, 67%) as a white solid, MS (ESI) calc'd for (C₁₇H₁₃F₃N₄O₃S) (M+1)⁺, 411.0. found 411.1. ¹H NMR (400 MHz, Methanol-d₄) δ 8.98 (s, 1H), 7.49-7.43 (m, 1H), 7.32 (s, 1H), 7.14-7.05 (m, 2H), 6.92-6.55 (m, 1H), 4.08 (s, 3H), 2.65 (s, 3H).

### Example 33

### 4-(5-chloro-2-(difluoromethoxy)phenyl)-N-(5-methoxy-1,3,4-thiadiazol-2-yl)-6-methylnicotinamide

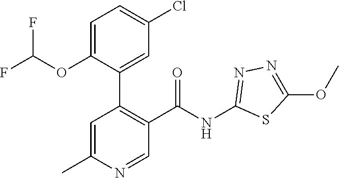 [SMILES: COc1nnc(NC(=O)c2cnc(C)cc2-c2cc(Cl)ccc2OC(F)F)s1]

### Step-1: 2-(5-chloro-2-(difluoromethoxy)phenyl)-4,4,5,5-tetramethyl-1,3,2-dioxaborolane

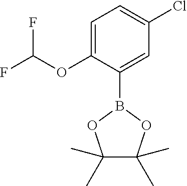 [SMILES: CC1(C)OB(c2cc(Cl)ccc2OC(F)F)OC1(C)C]

To a stirred solution of 2-bromo-4-chloro-1-(difluoromethoxy)benzene (1.0 g, 3.88 mmol) in 1,4-Dioxane (10 mL) were sequentially added potassium acetate (1.1 g, 11.65 mmol), 4,4,4′,4′,5,5,5′,5′-octamethyl-2,2′-bi(1,3,2-dioxaborolane) (1.2 g, 4.66 mmol) and PdCl2(dppf) (280.0 mg, 0.39 mmol) at 25° C. The resulting solution was stirred at 80° C. for 16 hr under nitrogen. The solvents were removed under vacuum. The resulting residue was purified by Combi Flash (Biotage Isolera Prime) which applied to 40 g silica gel column and eluted with 0˜25% ethyl acetate in petroleum ether within 30 min to afford 2-(5-chloro-2-(difluoromethoxy)phenyl)-4,4,5,5-tetramethyl-1,3,2-dioxaborolane (800.0 mg, 67%) as a brown solid. MS (ESI) calc'd for (C13H16BClF2O3) (M+1)+, 305.0. found 305.1.

### Step-2: methyl 4-(5-chloro-2-(difluoromethoxy)phenyl)-6-methylnicotinate

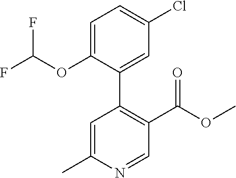 [SMILES: COC(=O)c1cnc(C)cc1-c1cc(Cl)ccc1OC(F)F]

To a stirred solution of 2-(5-chloro-2-(difluoromethoxy)phenyl)-4,4,5,5-tetramethyl-1,3,2-dioxaborolane (800.0 mg, 2.63 mmol) in 1,4-Dioxane (10 mL) and Water (0.5 mL) were sequentially added K2CO3 (1.1 g, 7.88 mmol), PdCl2(dppf) (192.0 mg, 0.26 mmol) and methyl 4-chloro-6-methylnicotinate (683.0 mg, 3.68 mmol) at 25° C. The resulting solution was stirred at 80° C. for 2 hr under nitrogen. The mixture was diluted with water. The aqueous layer was extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 40 g silica gel column and eluted with 0˜80% ethyl acetate in petroleum ether within 30 min to afford methyl 4-(5-chloro-2-(difluoromethoxy)phenyl)-6-methylnicotinate (1.2 g, 98%) as white solid. MS (ESI) calc'd for (C15H12ClF2NO3) (M+1)+, 328.0. found 328.1.

### Step-3: 4-(5-chloro-2-(difluoromethoxy)phenyl)-6-methylnicotinic Acid

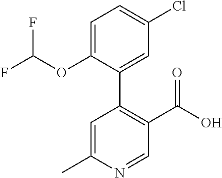 [SMILES: Cc1cc(-c2cc(Cl)ccc2OC(F)F)c(C(=O)O)cn1]

To a stirred solution of methyl 4-(5-chloro-2-(difluoromethoxy)phenyl)-6-methylnicotinate (1.2 g, 3.66 mmol) in Tetrahydrofuran (10 mL) and Water (5 mL) was added lithium hydroxide (87.8 mg, 3.66 mmol) at 25° C. The resulting solution was stirred at 25° C. for 1 hr. The organic solvent was removed under vacuum. The aqueous layer was acidified with 2 N HCl to pH ˜4 and extracted with ethyl acetate. The combined organic layers were washed with brine, dried over anhydrous sodium sulfate, filtered, and concentrated under vacuum to afford 4-(5-chloro-2-(difluoromethoxy)phenyl)-6-methylnicotinic acid (900 mg, crude) as a light-yellow oil. MS (ESI) calc'd for (C14H10ClF2NO3) (M+1)+, 314.0. found 314.0.

### Step-4: 4-(5-chloro-2-(difluoromethoxy)phenyl)-N-(5-methoxy-1,3,4-thiadiazol-2-yl)-6-methylnicotinamide

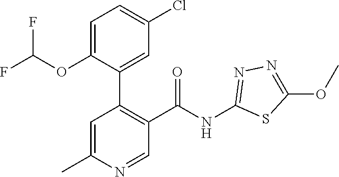 [SMILES: COc1nnc(NC(=O)c2cnc(C)cc2-c2cc(Cl)ccc2OC(F)F)s1]

To a stirred solution of 4-(5-chloro-2-(difluoromethoxy)phenyl)-6-methylnicotinic acid (200.0 mg, 0.57 mmol) in Acetonitrile (2 mL) were sequentially added 1-methyl-1H-imidazole (236 mg, 2.87 mmol) and 5-methoxy-1,3,4-thiadiazol-2-amine (77.1 mg, 0.57 mmol) at 25° C. To the above solution was added a solution of TCFH (161.0 mg, 0.57 mmol) at 25° C. under nitrogen. The resulting mixture was then stirred at 25° C. for 2 hr. The resulting residue was dissolved in DMF (3 mL) which was applied to a 40 g C18 column and purified by Combi Flash (Biotage Isolera Prime), eluted with 5˜100% acetonitrile in water within 40 min to afford 4-(5-chloro-2-(difluoromethoxy)phenyl)-N-(5-methoxy-1,3,4-thiadiazol-2-yl)-6-methylnicotinamide (52.0 mg, 21%) as a white solid. MS (ESI) calc'd for (C₁₇H₁₃ClF₂N₄O₃S) (M+1)⁺, 427.0. found 427.0. ¹H NMR (400 MHz, DMSO-d₆) δ 12.93 (s, 1H), 8.85 (s, 1H), 7.60-7.51 (m, 2H), 7.38 (s, 1H), 7.31-7.21 (m, 1H), 7.04-6.82 (m, 1H), 4.07 (s, 3H), 2.59 (s, 3H).

### Example 34

### 4-(2,6-dichlorophenyl)-N-(5-methoxy-1,3,4-thiadiazol-2-yl)-6-methylnicotinamide

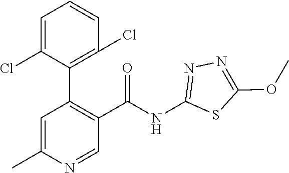 [SMILES: COc1nnc(NC(=O)c2cnc(C)cc2-c2c(Cl)cccc2Cl)s1]

### Step-1: methyl 4-(2,6-dichlorophenyl)-6-methylnicotinate

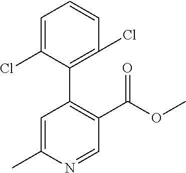 [SMILES: COC(=O)c1cnc(C)cc1-c1c(Cl)cccc1Cl]

To a stirred solution of methyl 4-chloro-6-methylnicotinate (500.0 mg, 2.69 mmol) and (2,6-dichlorophenyl)boronic acid (1.0 g, 5.39 mmol) in Toluene (5 mL) were sequentially added tris(di benzylideneacetone)dipalladium(0) (247.0 mg, 0.27 mmol) and K₂CO₃ (1.1 g, 8.08 mmol) at 23° C. The resulting solution was stirred at 100° C. for 16 hr under nitrogen atmosphere. The suspension was filtered. The filtrate was collected and concentrated under vacuum. The resulting residue was dissolved in DCM (2 mL) and purified by Combi Flash (Biotage Isolera Prime) which applied to a 40 g silica gel column that was eluted with 0˜20% ethyl acetate in petroleum ether within 30 min to afford methyl 4-(2,6-dichlorophenyl)-6-methylnicotinate (498.0 mg, 61%) as a yellow solid. MS (ESI) calculated for (C₁₄H₁₁Cl₂NO₂) (M+1)⁺, 296.0. found, 296.0.

### Step-2: 4-(2,6-dichlorophenyl)-6-methylnicotinic Acid

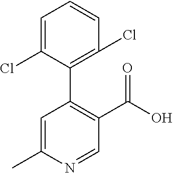 [SMILES: Cc1cc(-c2c(Cl)cccc2Cl)c(C(=O)O)cn1]

To a stirred solution of methyl 4-(2,6-dichlorophenyl)-6-methylnicotinate (490.0 mg, 1.66 mmol) in Methanol (3 mL) was added sodium hydroxide (265.0 mg, 6.62 mmol) in water (3 mL) at 20° C. The resulting solution was stirred at 80° C. for 30 min. The organic solvent was removed under vacuum. The aqueous layer was acidified with citric acid to pH 5˜6 and extracted with ethyl acetate. The combined organic layers were washed with brine, dried over anhydrous sodium sulfate, filtered, and concentrated under vacuum to afford 4-(2,6-dichlorophenyl)-6-methylnicotinic acid (430.0 mg, crude) as a yellow solid. MS (ESI) calculated for (C₁₃H₉Cl₂NO₂) (M+1)⁺, 282.0. found, 282.0.

### Step-3: 4-(2,6-dichlorophenyl)-N-(5-methoxy-1,3,4-thiadiazol-2-yl)-6-methylnicotinamide

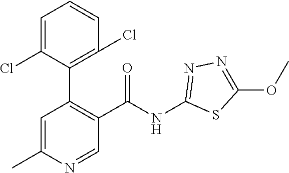 [SMILES: COc1nnc(NC(=O)c2cnc(C)cc2-c2c(Cl)cccc2Cl)s1]

To a stirred solution of 4-(2,6-dichlorophenyl)-6-methylnicotinic acid (300.0 mg, 1.06 mmol) and 5-methoxy-1,3,4-thiadiazol-2-amine (139.0 mg, 1.06 mmol) in acetonitrile (3 mL) was added 1-methylimidazole (437.0 mg, 5.32 mmol). To the above solution was added a solution of TCFH (298.0 mg, 1.06 mmol) in acetonitrile at 20° C. under nitrogen. The resulting solution was stirred at 20° C. for 2 hr. The resulting mixture was dissolved in DMF (3 mL) and was purified by prep-HPLC with the following condition: (Column: Atlantis HILIC OBD Column, 19*150 mm*5 μm; Mobile Phase A: Water (10 mmol/L NH₄HCO₃), Mobile Phase B: ACN; Flow rate: 60 mL/min; Gradient: 27% B to 48% B in 8 min, 48% B; Wave Length: 254 nm; RT1 (min): 7.8) to afford 4-(2,6-dichlorophenyl)-N-(5-methoxy-1,3,4-thiadiazol-2-yl)-6-methylnicotinamide (29.1 mg, 7% yield) as a white solid. MS (ESI) calculated for (C₁₆H₁₂Cl₂N₄O₂S) (M+1)⁺, 395.0. found, 395.1. ¹H NMR (400 MHz, DMSO-d₆) δ 13.07 (s, 1H), 9.04 (s, 1H), 7.65-7.55 (m, 2H), 7.48-7.40 (m, 1H), 7.33-7.20 (m, 1H), 4.06 (s, 3H), 2.61 (s, 3H).

### Example 35

### 4-(2-chloro-6-methoxyphenyl)-N-(5-methoxy-1,3,4-thiadiazol-2-yl)-6-methylnicotinamide

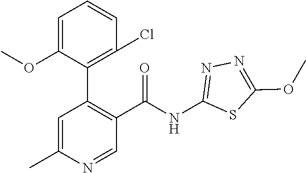

### Step-1: methyl 4-(2-chloro-6-methoxyphenyl)-6-methylnicotinate

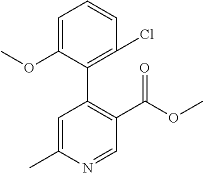

To a stirred solution of methyl 4-chloro-6-methylnicotinate (500.0 mg, 2.69 mmol) in 1,4-Dioxane (5 mL) were sequentially added water (1.0 mL), K₂CO₃ (1.1 g, 8.08 mmol) and Pd(dppf)Cl₂ (220.0 mg, 0.27 mmol) at 17° C. The resulting solution was stirred at 80° C. for 1 hr under nitrogen. The suspension was filtered. The filtrate was collected and concentrated under vacuum. The resulting residue was dissolved in DMF (3 mL) which was applied to a 40 g C18 column and purified by Combi Flash (Biotage Isolera Prime), eluted with 5˜60% acetonitrile in water within 35 min to afford methyl 4-(2-chloro-6-methoxyphenyl)-6-methylnicotinate (635.0 mg, 80%) as a yellow oil. MS (ESI) calculated for (C₁₅H₁₄ClNO₃) (M+1)⁺, 292.0. found, 292.2.

### Step-2: 4-(2-chloro-6-methoxyphenyl)-6-methylnicotinic Acid

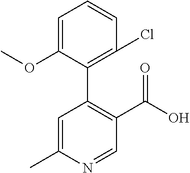 [SMILES: COc1cccc(Cl)c1-c1cc(C)ncc1C(=O)O]

To a stirred solution of methyl 4-(2-chloro-6-methoxyphenyl)-6-methylnicotinate (635.0 mg, 2.18 mmol) in methanol (4 mL) were sequentially added water (4 mL) and NaOH (348.0 mg, 8.71 mmol) at 17° C. The resulting solution was stirred at 80° C. for 30 min before diluted with water. The aqueous layer was acidified with citric acid to pH ˜6 and extracted with ethyl acetate. The combined organic layers were washed with brine, dried over anhydrous sodium sulfate, filtered, and concentrated under vacuum to afford 4-(2-chloro-6-methoxyphenyl)-6-methylnicotinic acid (600.0 mg, crude) as a brown solid. MS (ESI) calculated for (C₁₄H₁₂ClNO₃) (M+1)⁺, 278.0. found, 278.0.

### Step-3: 4-(2-chloro-6-methoxyphenyl)-N-(5-methoxy-1,3,4-thiadiazol-2-yl)-6-methylnicotinamide

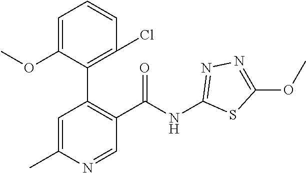

To a stirred solution of 4-(2-chloro-6-methoxyphenyl)-6-methylnicotinic acid (300.0 mg, 1.08 mmol) in acetonitrile (2 mL) were sequentially added 5-methoxy-1,3,4-thiadiazol-2-amine (156.0 mg, 1.19 mmol) and 1-Methylimidazole (443.0 mg, 5.40 mmol) at 17° C. To the above solution was added a solution of TCFH (303.0 mg, 1.08 mmol) in acetonitrile (1 mL) at 17° C. under nitrogen. The resulting mixture was then stirred at 17° C. for 1 hr. The solvents were removed under vacuum. The resulting residue was dissolved in DCM (3 mL) and purified by Combi Flash (Biotage Isolera Prime) which applied to a 40 g silica gel column that was eluted with 0˜10% methanol in dichloromethane within 20 min to afford 4-(2-chloro-6-methoxyphenyl)-N-(5-methoxy-1,3,4-thiadiazol-2-yl)-6-methylnicotinamide (86.1 mg, 20%) as a white solid. MS (ESI) calculated for (C₁₇H₁₅ClN₄O₃S) (M+1)⁺, 391.0. found, 391.15. ¹H NMR (400 MHz, DMSO-d₆) δ 12.88 (s, 1H), 8.86 (s, 1H), 7.39-7.35 (m, 1H), 7.25 (s, 1H), 7.14-7.11 (m, 1H), 7.04-7.02 (m, 1H), 4.06 (s, 3H), 3.60 (s, 3H), 2.57 (s, 3H).

### Example 36

### 4-(2-(difluoromethoxy)phenyl)-N-(5-methoxy-1,3,4-thiadiazol-2-yl)-6-methylnicotinamide

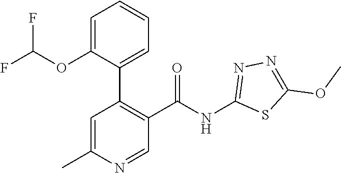 [SMILES: COc1nnc(NC(=O)c2cnc(C)cc2-c2ccccc2OC(F)F)s1]

### Step-1: methyl 4-(2-(difluoromethoxy)phenyl)-6-methylnicotinate

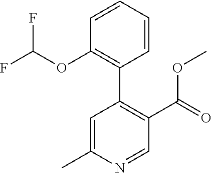 [SMILES: COC(=O)c1cnc(C)cc1-c1ccccc1OC(F)F]

To a stirred solution of methyl 4-chloro-6-methylnicotinate (1.0 g, 5.39 mmol) in 1,4-dioxane (5 mL) were sequentially added Bis (pinacolato) diboron (2.7 g, 10.78 mmol), potassium acetate (1.6 g, 16.16 mmol) and (1,1′-Bis(diphenylphosphino)ferrocene)dichloropalladium(II) (1.2 g, 1.62 mmol) at 23° C. The resulting solution was stirred at 100° C. for 4 hr under nitrogen. To the above mixture were added a mixture of 1-bromo-2-(difluoromethoxy)benzene (1.0 g, 4.50 mmol), water (2 mL), potassium carbonate (1.9 g, 13.50 mmol) and (1,1′-Bis(diphenylphosphino)ferrocene)dichloropalladium(II) (800.0 mg, 1.09 mmol) in dioxane (10 mL) at 23° C. The resulting solution was stirred at 80° C. for 2 hr under nitrogen. The suspension was filtered. The filtrate was collected and concentrated under vacuum. The resulting residue was purified by Combi Flash (Biotage Isolera Prime) which applied to 80.0 g silica gel column and eluted with 0˜14% methanol in dichloromethane within 40 min to afford methyl 4-(2-(difluoromethoxy)phenyl)-6-methylnicotinate (1.1 g, 97% yield) as a brown oil. MS (ESI) calc'd for (C₁₅H₁₃F₂NO₃) (M+1)⁺, 294.1. found, 294.1.

### Step-2: 4-(2-(difluoromethoxy)phenyl)-6-methylnicotinic Acid

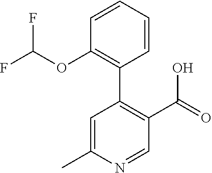 [SMILES: Cc1cc(-c2ccccc2OC(F)F)c(C(=O)O)cn1]

To a stirred solution of methyl 4-(2-(difluoromethoxy)phenyl)-6-methylnicotinate (1.0 g, 3.41 mmol) in methanol (4 mL) were added water (4 mL) and sodium hydroxide (546.0 mg, 13.64 mmol) at 20° C. The resulting solution was stirred at 20° C. for 2 hr under nitrogen. The organic solvent was removed under vacuum. The aqueous layer was acidified with sat. citric acid solution to pH ˜6 and extracted with ethyl acetate. The combined organic layers were washed with brine, dried over anhydrous sodium sulfate, filtered, and concentrated under vacuum to afford 4-(2-(difluoromethoxy)phenyl)-6-methylnicotinic acid (823.0 mg, 70% yield) as a brown solid. MS (ESI) calc'd for (C₁₄H₁₁F₂NO₃) (M+1)⁺, 280.1. found 280.1.

### Step-3: 4-(2-(difluoromethoxy)phenyl)-N-(5-methoxy-1,3,4-thiadiazol-2-yl)-6-methylnicotinamide

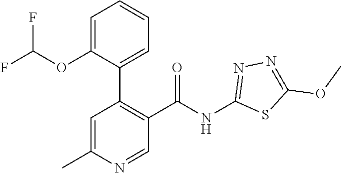 [SMILES: COc1nnc(NC(=O)c2cnc(C)cc2-c2ccccc2OC(F)F)s1]

To a stirred solution of 4-(2-(difluoromethoxy)phenyl)-6-methylnicotinic acid (200.0 mg, 0.72 mmol) and 5-methoxy-1,3,4-thiadiazol-2-amine (103.0 mg, 0.79 mmol) in acetonitrile (2 mL) was added 1-methylimidazole (293.5 mg, 3.58 mmol). Then TCFH (201.0 mg, 0.72 mmol) in acetonitrile (1 mL) was added to the above mixture at 20° C. The resulting solution was stirred at 20° C. for 2 hr under nitrogen. The resulting residue was dissolved in acetonitrile (1 mL) which was applied to a 20 g C18 column and purified by Combi Flash (Biotage Isolera Prime), eluted with 5˜40% acetonitrile in water within 30 min to afford 4-(2-(difluoromethoxy)phenyl)-N-(5-methoxy-1,3,4-thiadiazol-2-yl)-6-methylnicotinamide (143.0 mg, 50% yield) as a white solid. MS (ESI) calc'd for (C₁₇H₁₄F₂N₄O₃S) (M+1)⁺, 393.1. found 393.1. ¹H NMR (400 MHz, DMSO-d₆) δ 12.90 (s, 1H), 8.81 (s, 1H), 7.53-7.39 (m, 2H), 7.37-7.30 (m, 2H), 7.23-6.84 (m, 2H), 4.06 (s, 3H), 2.59 (s, 3H).

### Example 37

### 2′-chloro-5′-methoxy-6-methyl-N-(5-propoxy-1,3,4-thiadiazol-2-yl)-(4,4′-bipyridine)-3-carboxamide

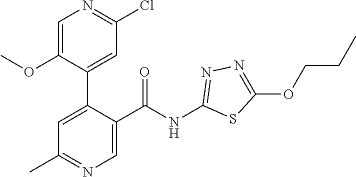 [SMILES: CCCOc1nnc(NC(=O)c2cnc(C)cc2-c2cc(Cl)ncc2OC)s1]

### Step-1: O-propyl Hydrazinecarbothioate

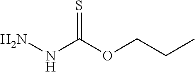

To a solution of potassium O-propyl carbonodithioate (2.0 g, 11.47 mmol) in Methanol (15 mL) was added hydrazine (0.5 mL, 13.77 mmol) at 16° C. The resulting solution was stirred at 16° C. for 2 hr. The reaction mixture was quenched by the addition of water and extracted with ethyl acetate. The combined organic layers were washed with brine, dried over anhydrous sodium sulfate, filtered, and concentrated under vacuum to afford O-propyl hydrazinecarbothioate (2.3 g, crude) as a yellow oil.

### Step-2: 5-propoxy-1,3,4-thiadiazol-2-amine

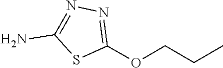

To a solution of O-propyl hydrazinecarbothioate (2.0 g, 8.94 mmol) in Methanol (10 mL) was added TEA (1.2 mL, 8.94 mmol) and cyanic bromide (1.0 g, 9.84 mmol) at 20° C. The resulting solution was stirred at 20° C. for 30 min. The reaction mixture was quenched by the addition of water and extracted with ethyl acetate. The combined organic layers were washed with brine, dried over anhydrous sodium sulfate, filtered, and concentrated under vacuum. The residue was dissolved in DMF (4 mL) which was applied to a 40 g C18 column and purified by Combi Flash (Biotage Isolera Prime), eluted with 5˜24% acetonitrile in water within 43 min to afford 5-propoxy-1,3,4-thiadiazol-2-amine (97.0 mg, 6%) as a yellow solid. MS (ESI) calc'd for (C₅H₉N₃OS) (M+1)⁺, 160.0. found 160.0.

### Step-3: 2′-chloro-5′-methoxy-6-methyl-N-(5-propoxy-1,3,4-thiadiazol-2-yl)-(4,4′-bipyridine)-3-carboxamide

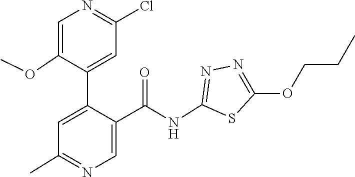 [SMILES: CCCOc1nnc(NC(=O)c2cnc(C)cc2-c2cc(Cl)ncc2OC)s1]

To a mixture of 5-propoxy-1,3,4-thiadiazol-2-amine (89.0 mg, 0.49 mmol) in Acetonitrile (4 mL) were added Intermediate G (120.0 mg, 0.40 mmol), and 1-methyl-1H-imidazole (101.0 mg, 1.22 mmol). A solution of TCFH (172.0 mg, 0.61 mmol) in Acetonitrile (1 mL) was added thereto dropwise under nitrogen. The resulting solution was stirred at 20° C. for 2 hr. The organic solvent was removed under vacuum. The residue was dissolved in DMF (5 mL) and was purified by prep-HPLC with the following condition: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 40% B in 8 min, 40% B; Wave Length: 254 nm; RT1 (min): 7.82) to afford 2′-chloro-5′-methoxy-6-methyl-N-(5-propoxy-1,3,4-thiadiazol-2-yl)-(4,4′-bipyridine)-3-carboxamide (102.9 mg, 59%) as a white solid. MS (ESI) calc'd for (C₁₈H₁₈ClN₅O₃S) (M+1)⁺, 420.0. found 420.1. ¹H NMR (400 MHz, DMSO-d₆) δ 12.89 (s, 1H), 8.83 (s, 1H), 8.16 (s, 1H), 7.51 (s, 1H), 7.40 (s, 1H), 4.33-4.37 (t, J=6.4 Hz, 2H), 3.63 (s, 3H), 2.58 (s, 3H), 1.73-1.81 (m, 2H), 0.94-0.98 (t, J=7.6 Hz, 3H).

### Example 38

### 4-(2-chloro-6-fluorophenyl)-N-(5-methoxy-1,3,4-thiadiazol-2-yl)-6-methylnicotinamide

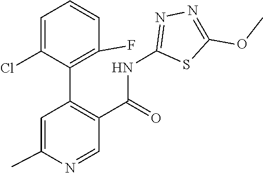

### Step 1 methyl 4-(2-chloro-6-fluorophenyl)-6-methylnicotinate

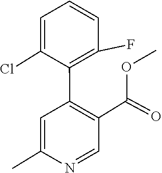 [SMILES: COC(=O)c1cnc(C)cc1-c1c(F)cccc1Cl]

To a stirred solution of (2-chloro-6-fluorophenyl)boronic acid (2.0 g, 11.47 mmol) in 1,4-Dioxane (10 mL) were added methyl 4-chloro-6-methylnicotinate (532.2 mg, 2.87 mmol), potassium carbonate (1.1 g, 8.60 mmol) and Water (2 mL) at 23° C. To the above solution was added Pd(dtbpf)Cl₂ (187.2 mg, 0.28 mmol) at 23° C. under nitrogen. The resulting mixture was then stirred at 80° C. under nitrogen for 2 hr. The reaction mixture was dilution by the addition of water and extracted with ethyl acetate. The combined organic layers were washed with brine, dried over anhydrous sodium sulfate, filtered, and concentrated under vacuum. The resulting residue was dissolved in DCM (5 mL) and purified by Combi Flash (Biotage Isolera Prime) which applied to a 80 g silica gel column that was eluted with 0˜60% ethyl acetate in petroleum ether within 30 min to afford methyl 4-(2-chloro-6-fluorophenyl)-6-methylnicotinate (200.0 mg 24%) as a yellow oil. MS (ESI) calc'd for (C₁₄H₁₁ClFNO₂) (M+1)⁺, 280.0. found, 280.0.

### Step 2 4-(2-chloro-6-fluorophenyl)-6-methylnicotinic Acid

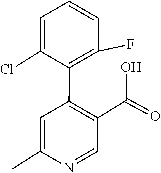 [SMILES: Cc1cc(-c2c(F)cccc2Cl)c(C(=O)O)cn1]

To a stirred solution of methyl 4-(2-chloro-6-fluorophenyl)-6-methylnicotinate (200.0 mg, 0.72 mmol) in Tetrahydrofuran (1 mL) and Water (1 mL) was added LiOH (34.2 mg, 1.44 mmol) at 23° C. The resulting solution was stirred at 23° C. for 1 hr before diluted with water. The aqueous layer was acidified with citric acid to pH ˜3 and extracted with ethyl acetate. The combined organic layers were washed with brine, dried over anhydrous sodium sulfate, filtered, and concentrated under vacuum. The resulting residue was dissolved in DMF (2 mL) which was applied to a 20 g C18 column and purified by Combi Flash (Biotage Isolera Prime), eluted with 5˜60% acetonitrile in water within 25 min to afford 4-(2-chloro-6-fluorophenyl)-6-methylnicotinic acid (100.0 mg, 50%) as a white solid. MS (ESI) calc'd for (C₁₃H₉ClFNO₂) (M+1)⁺, 266.0. found, 266.1.

### Step 3 4-(2-chloro-6-fluorophenyl)-N-(5-methoxy-1,3,4-thiadiazol-2-yl)-6-methylnicotinamide

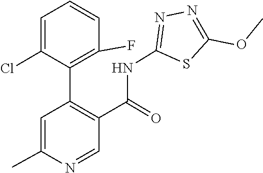

To a stirred solution of 4-(2-chloro-6-fluorophenyl)-6-methylnicotinic acid (80.0 mg, 0.30 mmol) in Acetonitrile (2 mL) were added 5-methoxy-1,3,4-thiadiazol-2-amine (39.5 mg, 0.30 mmol) and 1-methylimidazole (124.2 mg, 1.50 mmol) at 23° C. To the above solution was added a solution of TCFH (85.0 mg, 0.30 mmol) in Acetonitrile (0.5 mL) at 23° C. under nitrogen. The resulting mixture was then stirred at 23° C. for 2 hr under nitrogen. The mixture was dissolved in DMF (4 mL) and purified by prep-HPLC with the following conditions: (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 25% B to 45% B in 8 min, 45% B to 95% B in 8.2 min, 95% B to 95% B in 9.5 min, 95% B to 5% B in 11 min, 5% B; Wave Length: 254 nm; RT1 (min): 7; Injection Volume: 1.5 mL; Number Of Runs: 4) to afford 4-(2-chloro-6-fluorophenyl)-N-(5-methoxy-1,3,4-thiadiazol-2-yl)-6-methylnicotinamide (51.3 mg, 45%) as a white solid. MS (ESI) calc'd for (C₁₆H₁₂ClFN+O₂S) (M+1)⁺, 379.0. found, 379.0. ¹H NMR (400 MHz, DMSO-d₆) δ 13.09 (s, 1H), 9.01 (s, 1H), 7.53-7.28 (m, 4H), 4.05 (s, 3H), 2.60 (s, 3H).

### Example 39

### 2′-chloro-N-(5-methoxy-1,3,4-thiadiazol-2-yl)-5′,6-dimethyl-(4,4′-bipyridine)-3-carboxamide

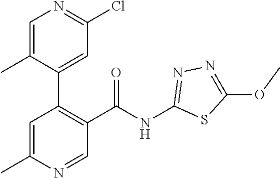

### Step-1: methyl 2′-chloro-5′,6-dimethyl-(4,4′-bipyridine)-3-carboxylate

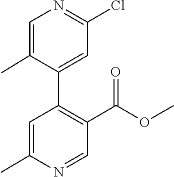

To a solution of methyl 4-chloro-6-methylnicotinate (1.5 g, 7.89 mmol) in 1,4-Dioxane (5 mL) and Water (1 mL) were added methyl 6-methyl-4-(4,4,5,5-tetramethyl-1,3,2-dioxaborolan-2-yl)nicotinate (4.4 g, 15.88 mmol), K₂CO₃ (1.6 g, 11.8 mmol) and PdCl2(dppf) (0.8 g, 1.19 mmol) at 20° C. under nitrogen. The resulting solution was stirred at 80° C. for 2 hr under nitrogen. The suspension was filtered. The filtrate was collected and concentrated under vacuum. The resulting residue was purified by Combi Flash (Biotage Isolera Prime) which applied to 40 g silica gel column and eluted with 0˜50% ethyl acetate in petroleum ether within 35 min to afford methyl 2′-chloro-5′,6-dimethyl-(4,4′-bipyridine)-3-carboxylate (1.1 g, 96%) as a yellow solid. MS (ESI) calc'd for (C₁₄H₁₃ClN₂O₂) (M+1)⁺, 277.1. found, 277.1.

### Step-2: 2′-chloro-5′,6-dimethyl-(4,4′-bipyridine)-3-carboxylic Acid

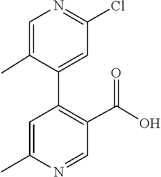 [SMILES: Cc1cc(-c2cc(Cl)ncc2C)c(C(=O)O)cn1]

To a stirred solution of methyl 2′-chloro-5′,6-dimethyl-(4,4′-bipyridine)-3-carboxylate (500.0 mg, 1.81 mmol) in Tetrahydrofuran (2 mL) was added a solution of lithium hydroxide (43.3 mg, 1.81 mmol) in Water (2 mL) at 20° C. The resulting solution was stirred at 20° C. for 1 hr. The organic solvent was removed under vacuum. The aqueous layer was acidified with citric acid to pH ˜5 and extracted with ethyl acetate. The combined organic layers were washed with brine, dried over anhydrous sodium sulfate, filtered, and concentrated under vacuum to afford 2′-chloro-5′,6-dimethyl-(4,4′-bipyridine)-3-carboxylic acid (384.0 mg, crude) as a yellow solid. MS (ESI) calc'd for (C₁₃H₁₁ClN₂O₂) (M+1)⁺, 263.1. found, 263.1.

### Step-3: 2′-chloro-N-(5-methoxy-1,3,4-thiadiazol-2-yl)-5′,6-dimethyl-(4,4′-bipyridine)-3-carboxamide

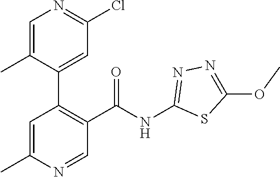

To a solution of 2′-chloro-5′,6-dimethyl-(4,4′-bipyridine)-3-carboxylic acid (150.0 mg, 0.57 mmol) in Acetonitrile (2 mL) were added 5-methoxy-1,3,4-thiadiazol-2-amine (74.9 mg, 0.57 mmol) and 1-methylimidazole (234.5 mg, 2.86 mmol) at 20° C. under nitrogen. To the above solution was added a solution of TCFH (160.0 mg, 0.57 mmol) in Acetonitrile (2 mL) at 20° C. under nitrogen. The mixture was then stirred at 20° C. for 1 hr. The resulting mixture was dissolved with DMF (1 mL) which was applied to a 20 g C18 column and purified by Combi Flash (Biotage Isolera Prime), eluted with 5˜55% acetonitrile in water within 30 min to afford 2′-chloro-N-(5-methoxy-1,3,4-thiadiazol-2-yl)-5′,6-dimethyl-(4,4′-bipyridine)-3-carboxamide (48.5 mg, 22%) as a white solid. MS (ESI) calc'd for (C₁₆H₁₄ClN₅O₂S) (M+1)⁺, 376.1. found 376.1. ¹H NMR (400 MHz, DMSO-d₆) δ 13.05 (s, 1H), 8.98 (s, 1H), 8.30 (s, 1H), 7.29-7.36 (m, 2H), 4.04 (s, 3H), 2.58 (s, 3H), 1.98 (s, 3H).

### Example 40

### 4-(3,5-dimethyl-1H-pyrazol-4-yl)-N-(5-methoxy-1,3,4-thiadiazol-2-yl)-6-methylnicotinamide

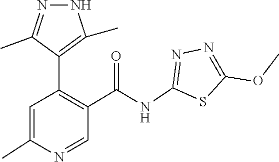

### Step-1: methyl 4-(3,5-dimethyl-1H-pyrazol-4-yl)-6-methylnicotinate

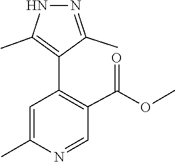

To a stirred solution of methyl 4-chloronicotinate (500.0 mg, 2.91 mmol) in 1,4-Dioxane (5 mL) were sequentially added 3,5-dimethyl-4-(4,4,5,5-tetramethyl-1,3,2-dioxaborolan-2-yl)-1H-pyrazole (971.0 mg, 4.37 mmol), water (1.0 mL), K₂CO₃ (1.2 g, 8.74 mmol) and Pd(dppf)Cl₂ (238.0 mg, 0.29 mmol) at 17° C. The resulting solution was stirred at 80° C. for 16 hr under nitrogen. The suspension was filtered. The filtrate was collected and concentrated under vacuum. The resulting residue was purified by Combi Flash (Biotage Isolera Prime) which applied to 80.0 g silica gel column and eluted with 0˜10% methanol in dichloromethane within 25 min to afford methyl 4-(3,5-dimethyl-1H-pyrazol-4-yl)-6-methylnicotinate (202.0 mg, 26%) as a red solid. MS (ESI) calculated for (C₁₃H₁₅N₃O₂) (M+1)⁺, 246.0. found, 246.2.

### Step-2: 4-(3,5-dimethyl-4H-pyrazol-4-yl)-6-methylnicotinic Acid

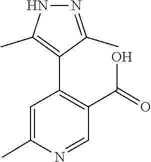 [SMILES: Cc1cc(-c2c(C)n[nH]c2C)c(C(=O)O)cn1]

To a stirred solution of methyl 4-(3,5-dimethyl-4H-pyrazol-4-yl)-6-methylnicotinate (160.0 mg, 0.65 mmol) in methanol (2 mL) was added a solution of NaOH (104.0 mg, 2.61 mmol) in water (2 mL) at 17° C. The resulting solution was stirred at 17° C. for 30 min before diluted with water. The organic solvent was removed under vacuum. The aqueous layer was acidified with citric acid to pH ˜6 and extracted with ethyl acetate. The combined organic layers were washed with brine, dried over anhydrous sodium sulfate, filtered, and concentrated under vacuum. The resulting residue was dissolved in DMF (2 mL) which was applied to a 40 g C18 column and purified by Combi Flash (Biotage Isolera Prime), eluted with 5˜7% acetonitrile in water within 20 min to afford 4-(3,5-dimethyl-4H-pyrazol-4-yl)-6-methylnicotinic acid (42.0 mg, 27%) as a white solid. MS (ESI) calculated for (C₁₂H₁₃N₃O₂) (M+1)⁺, 231.0. found, 231.

### Step-3: 4-(3,5-dimethyl-1H-pyrazol-4-yl)-N-(5-methoxy-1,3,4-thiadiazol-2-yl)-6-methylnicotinamide

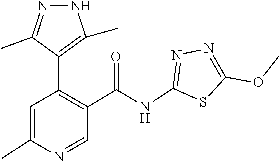

To a stirred solution of 4-(3,5-dimethyl-1H-pyrazol-4-yl)-6-methylnicotinic acid (140.0 mg, 0.61 mmol) in acetonitrile (2 mL) were sequentially added 5-methoxy-1,3,4-thiadiazol-2-amine (87.0 mg, 0.67 mmol) and 1-Methylimidazole (249.0 mg, 3.03 mmol) at 17° C. To the above solution was added a solution of TCFH (170.0 mg, 0.61 mmol) in acetonitrile (1 mL) at 17° C. under nitrogen. The resulting mixture was then stirred at 17° C. for 1 hr. The organic solvent was removed under vacuum. The residue was dissolved in DMF (3 mL) and was purified by prep-HPLC with the following condition: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 7% B to 27% B in 8 min, 27% B; Wave Length: 254 nm; RT1 (min): 7) to afford 4-(3,5-dimethyl-1H-pyrazol-4-yl)-N-(5-methoxy-1,3,4-thiadiazol-2-yl)-6-methylnicotinamide (60.4 mg, 29%) as a white solid. MS (ESI) calculated for (C₁₅H₁₆N₆O₂S) (M+1)⁺, 345.0. found, 345.1. ¹H NMR (400 MHz, DMSO-d₆) δ 12.38 (s, 1H), 8.68 (s, 1H), 7.18 (s, 1H), 4.06 (s, 3H), 2.59 (s, 3H), 2.08-1.87 (m, 6H).

### Example 41

### 5′-chloro-N-(5-methoxy-1,3,4-thiadiazol-2-yl)-2′,6-dimethyl-(4,4′-bipyridine)-3-carboxamide

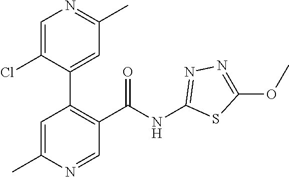 [SMILES: COc1nnc(NC(=O)c2cnc(C)cc2-c2cc(C)ncc2Cl)s1]

### Step-1: methyl 2′,5′-dichloro-6-methyl-(4,4′-bipyridine)-3-carboxylate

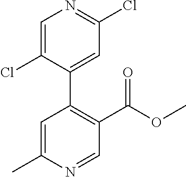 [SMILES: COC(=O)c1cnc(C)cc1-c1cc(Cl)ncc1Cl]

To a stirred solution of (2,5-dichloropyridin-4-yl)boronic acid (0.5 g, 2.69 mmol) in 1,4-Dioxane (10 mL) were sequentially added methyl 4-chloro-6-methylnicotinate (1.0 g, 5.39 mmol), PdCl2(DTBPF) (0.3 g, 0.53 mmol) and K₂CO₃ (2.2 g, 16.16 mmol) at 25° C. The resulting solution was stirred at 80° C. for 2 h under nitrogen. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 40 g silica gel column and eluted with 50˜80% ethyl acetate in petroleum ether within 30 min to afford methyl 2′,5′-dichloro-6-methyl-(4,4′-bipyridine)-3-carboxylate (400.0 mg, 20%) as a light-yellow solid. MS (ESI) calc'd for (C₁₃H₁₀Cl₂N₂O₂) (M+1)⁺, 297.0. found 297.0.

### Step-2: 5′-chloro-2′,6-dimethyl-(4,4′-bipyridine)-3-carboxylate

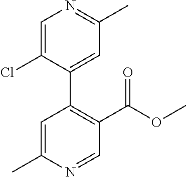 [SMILES: COC(=O)c1cnc(C)cc1-c1cc(C)ncc1Cl]

To a stirred solution of methyl 2′,5′-dichloro-6-methyl-(4,4′-bipyridine)-3-carboxylate (400.0 mg, 1.34 mmol) in Tetrahydrofuran (3 mL) was added trimethylaluminum (4 mL, 4.04 mmol) at 25° C. The resulting solution was stirred at 85° C. for 16 h. The reaction mixture was quenched by the addition of water and extracted with ethyl acetate. The combined organic layers were washed with brine, dried over anhydrous sodium sulfate, filtered, and concentrated under vacuum to afford crude product as a yellow oil. The resulting residue was purified by Combi Flash (Biotage Isolera Prime) which applied to 20 g silica gel column and eluted with 30-60% ethyl acetate in petroleum ether within 30 min to afford methyl 5′-chloro-2′,6-dimethyl-(4,4′-bipyridine)-3-carboxylate (270.0 mg, 58%) as a colorless oil. MS (ESI) calc'd for (C₁₄H₁₃ClN₂O₂) (M+1)⁺, 277.0. found 277.1.

### Step-3: 5′-chloro-2′,6-dimethyl-(4,4′-bipyridine)-3-carboxylic Acid

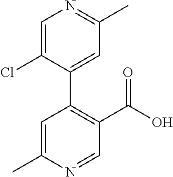 [SMILES: Cc1cc(-c2cc(C)ncc2C(=O)O)c(Cl)cn1]

To a stirred solution of methyl 5′-chloro-2′,6-dimethyl-(4,4′-bipyridine)-3-carboxylate (270.0 mg, 0.78 mmol) in Tetrahydrofuran (3 mL) and Water (1 mL) was added lithium hydroxide (18.6 mg, 0.78 mmol) at 25° C. The resulting solution was stirred at 25° C. for 1 h before diluted with water. The organic solvent was removed under vacuum. The aqueous layer was acidified with 2 N HCl to pH ˜4 and extracted with ethyl acetate. The combined organic layers were washed with brine, dried over anhydrous sodium sulfate, filtered, and concentrated under vacuum to afford 5′-chloro-2′,6-dimethyl-(4,4′-bipyridine)-3-carboxylic acid (150.0 mg, crude) as a white solid. MS (ESI) calc'd for (C₁₃H₁₁ClN₂O₂) (M+1), 263.0. found 263.0.

### Step-4: 5′-chloro-N-(5-methoxy-1,3,4-thiadiazol-2-yl)-2′,6-dimethyl-(4,4′-bipyridine)-3-carboxamide

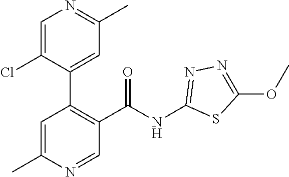 [SMILES: COc1nnc(NC(=O)c2cnc(C)cc2-c2cc(C)ncc2Cl)s1]

To a stirred solution of 5′-chloro-2′,6-dimethyl-(4,4′-bipyridine)-3-carboxylic acid (150.0 mg, 0.51 mmol) in Acetonitrile (2 mL) were sequentially added 1-methyl-1H-imidazole (211.0 mg, 2.57 mmol) and 5-methoxy-1,3,4-thiadiazol-2-amine (81.0 mg, 0.61 mmol) at 25° C. To the above solution was added TCFH (159.0 mg, 0.56 mmol) at 25° C. under nitrogen. The resulting mixture was then stirred at 25° C. for 2 hr. The suspension was filtered. The filter cake was washed by water (20 mL×3) and tert-Butyl methyl ether (20 mL). The solid was collected and dried under vacuum to afford 5′-chloro-N-(5-methoxy-1,3,4-thiadiazol-2-yl)-2′,6-dimethyl-(4,4′-bipyridine)-3-carboxamide (79.0 mg, 40%) as a light-yellow solid. MS (ESI) calc'd for (C₁₆H₁₄ClN₅O₂S) (M+1)⁺, 376.0. found 376.1. ¹H NMR (400 MHz, Methanol-d₄) δ 13.10 (s, 1H), δ 8.97 (s, 1H), 8.51 (s, 1H), 7.36-7.30 (m, 2H), 4.06 (s, 3H), 2.69 (s, 3H), 2.59 (s, 3H).

### Example 42

### 2′-chloro-5′-ethoxy-N-(5-methoxy-1,3,4-thiadiazol-2-yl)-6-methyl-(4,4′-bipyridine)-3-carboxamide

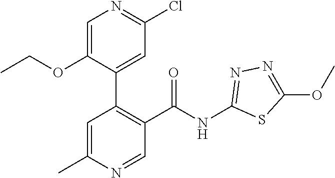 [SMILES: CCOc1cnc(Cl)cc1-c1cc(C)ncc1C(=O)Nc1nnc(OC)s1]

### Step 1 2-chloro-5-ethoxypyridine

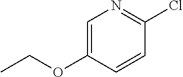

To a stirred solution of 6-chloropyridin-3-ol (1.0 g, 7.72 mmol) in N, N-Dimethylformamide (15 mL) were sequentially added K₂CO₃ (2.1 g, 15.44 mmol) and iodoethane (1.4 g, 8.97 mmol) at 20° C. The resulting solution was stirred at 40° C. for 2 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (4 mL) and purified by Combi Flash (Biotage Isolera Prime) which applied to a 40 g silica gel column that was eluted with 0˜20% ethyl acetate in petroleum ether within 30 min to afford 2-chloro-5-ethoxypyridine (827.0 mg, 67%) as a white solid. MS (ESI) calculated for (C₇H₈ClNO) (M+1)⁺, 158.0. found, 158.0.

### Step 2 (2-chloro-5-ethoxypyridin-4-yl)boronic Acid

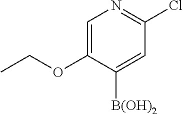

To a stirred solution of 2-chloro-5-ethoxypyridine (800.0 mg, 5.08 mmol) in Tetrahydrofuran (30 mL) was added lithium diisopropylamide (5 ml, 10.00 mmol, 2 N in THF) dropwise at −78° C. The resulting solution was stirred at −78° C. for 3 hr under nitrogen. To the above solution was added triisopropyl borate (1.9 g, 10.15 mmol) dropwise at −78° C. under nitrogen. The mixture was then stirred at −78° C. for 2 hr before acidified with HCl (2 N) to pH 4˜5 at 0° C. The resulting mixture was stirred at 0° C. to room temperature for 30 min, and extracted with ethyl acetate. The combined organic layers were washed with brine, dried over anhydrous sodium sulfate, filtered, and concentrated under vacuum to afford (2-chloro-5-ethoxypyridin-4-yl)boronic acid (650.0 mg, crude) as a yellow solid. MS (ESI) calculated for (C₇H₉BClNO₃) (M+1)⁺, 202.0. found, 202.0.

### Step 3 methyl 2′-chloro-5′-ethoxy-6-methyl-(4,4′-bipyridine)-3-carboxylate

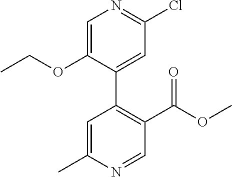 [SMILES: CCOc1cnc(Cl)cc1-c1cc(C)ncc1C(=O)OC]

To a stirred solution of methyl 4-chloro-6-methylnicotinate (500.0 mg, 2.69 mmol) in 1,4-Dioxane (7 mL) were sequentially added water (1.5 mL), (2-chloro-5-ethoxypyridin-4-yl)boronic acid (543.0 mg, 2.69 mmol), Dichloro(1,1′-bis(di-t-butylphosphino)ferrocene)palladium(II) (176.0 mg, 0.27 mmol) and K₂CO₃ (1.1 g, 8.08 mmol) at 23° C. The resulting solution was stirred at 80° C. for 3 h under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (3 mL) and purified by Combi Flash (Biotage Isolera Prime) which applied to a 40 g silica gel column, eluted with 0˜3% methanol in dichloromethane within 20 min to afford methyl 2′-chloro-5′-ethoxy-6-methyl-(4,4′-bipyridine)-3-carboxylate (360.0 mg, 43%) as a colorless oil. MS (ESI) calculated for (C₁₅H₁₅ClN₂O₃) (M+1)⁺, 307.1. found, 307.1.

### Step 4 2′-chloro-5′-ethoxy-6-methyl-(4,4′-bipyridine)-3-carboxylic Acid

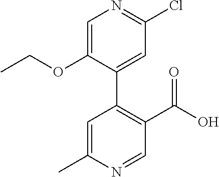 [SMILES: CCOc1cnc(Cl)cc1-c1cc(C)ncc1C(=O)O]

To a stirred solution of methyl 2′-chloro-5′-ethoxy-6-methyl-(4,4′-bipyridine)-3-carboxylate (350.0 mg, 1.14 mmol) in Methanol (3 mL) was added a solution of sodium hydroxide (183.0 mg, 4.56 mmol) in water (3 mL) at 20° C. The resulting solution was stirred at 80° C. for 1 hr. The mixture was diluted with water. The volatiles were removed under vacuum. The aqueous layer was acidified with citric acid to pH 5˜6 and extracted with ethyl acetate. The combined organic layers were washed with brine, dried over anhydrous sodium sulfate, filtered, and concentrated under vacuum to afford 2′-chloro-5′-ethoxy-6-methyl-(4,4′-bipyridine)-3-carboxylic acid (330.0 mg, crude) as a pink solid. MS (ESI) calculated for (C₁₄H₁₃ClN₂O₃) (M+1)⁺, 293.1. found, 293.1.

### Step 5 2′-chloro-5′-ethoxy-N-(5-methoxy-1,3,4-thiadiazol-2-yl)-6-methyl-(4,4′-bipyridine)-3-carboxamide

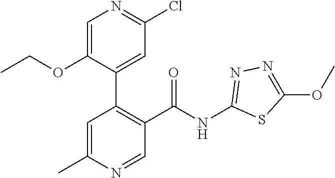 [SMILES: CCOc1cnc(Cl)cc1-c1cc(C)ncc1C(=O)Nc1nnc(OC)s1]

To a stirred solution of 2′-chloro-5′-ethoxy-6-methyl-(4,4′-bipyridine)-3-carboxylic acid (310.0 mg, 1.06 mmol) in acetonitrile (4 mL) were added 5-methoxy-1,3,4-thiadiazol-2-amine (153.0 mg, 1.17 mmol) and 1-methylimidazole (435.0 mg, 5.30 mmol). To the above solution was added a solution of TCFH (297.0 mg, 1.06 mmol) in acetonitrile (1 mL) at 20° C. under nitrogen. The resulting solution was stirred at 20° C. for 2 hr. The resulting mixture was dissolved in DMF (2 mL) which was applied to a 40 g C18 column and purified by Combi Flash (Biotage Isolera Prime), eluted with 5˜70% acetonitrile in water within 30 min to afford a yellow solid (88% purity). The product was dissolved in DMF (3 mL) and further purified by prep-HPLC with the following condition: (Column: XBridge Prep Phenyl OBD Column, 19*250 mm, 5 um; Mobile Phase A: Water (10 mmol/L NH₄HCO₃), Mobile Phase B: MeOH—HPLC; Flow rate: 25 mL/min; Gradient: 55% B to 65% B in 8 min, 65% B to 95% B in 8.2 min, 95% B to 95% B in 10 min, 95% B to 5% B in 11 min, 5% B; Wave Length: 220\254 nm; RT1 (min): 8; Injection Volume: 500 mL; Number Of Runs: 5) to afford 2′-chloro-5′-ethoxy-N-(5-methoxy-1,3,4-thiadiazol-2-yl)-6-methyl-(4,4′-bipyridine)-3-carboxamide (70.5 mg, 16%) as a white solid. MS (ESI) calculated for (C₁₇H₁₆ClN₅O₃S) (M+1)⁺, 406.1. found, 406.1. ¹H NMR (400 MHz, DMSO-d₆) δ 12.95 (s, 1H), 8.83 (s, 1H), 8.13 (s, 1H), 7.52 (s, 1H), 7.41 (s, 1H), 4.08 (s, 3H), 3.97-3.87 (m, 2H), 2.59 (s, 3H), 1.12-1.04 (m, 3H).

### Example 43

### N-(5-methoxy-1,3,4-thiadiazol-2-yl)-4-(3-methoxy-6-methylpyridazin-4-yl)-6-methylnicotinamide

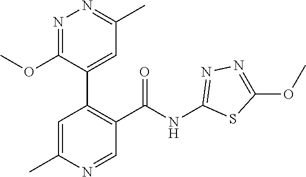

### Step 1: methyl 4-(6-chloro-3-methoxypyridazin-4-yl)-6-methylnicotinate

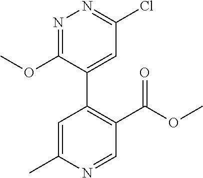

To a stirred solution of methyl 4-chloro-6-methylnicotinate (1.0 g, 5.39 mmol) in 1,4-dioxane (5 mL) were sequentially added Bis (pinacolato) diboron (2.7 g, 10.78 mmol), potassium acetate (1.6 g, 16.16 mmol) and (1,1′-Bis(diphenylphosphino)ferrocene)dichloropalladium(II) (1.2 g, 1.62 mmol) at 23° C. The resulting solution was stirred at 100° C. for 4 hr under nitrogen. To the above mixture were added water (0.4 mL), potassium carbonate (299.0 mg, 2.17 mmol) and (1,1′-Bis(diphenylphosphino)ferrocene)dichloropalladium(II) (158.0 mg, 0.22 mmol) in 1,4-dioxane (2 mL) at 23° C. The resulting solution was stirred at 80° C. for 16 hr under nitrogen. The suspension was filtered. The filtrate was collected and concentrated under vacuum. The resulting residue was dissolved in acetonitrile (2 mL) which was applied to a 40.0 g C18 column and purified by Combi Flash (Biotage Isolera Prime), eluted with 5˜38% acetonitrile in water within 30 min to afford methyl 4-(6-chloro-3-methoxypyridazin-4-yl)-6-methylnicotinate (114.0 mg, 50%) as a yellow oil. MS (ESI) calc'd for (C₁₃H₁₂ClN₃O₃) (M+1)⁺, 294.1. found, 294.1.

### Step-2: methyl 4-(3-methoxy-6-methylpyridazin-4-yl)-6-methylnicotinate

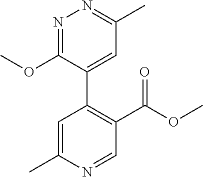

To a stirred solution of methyl 4-(6-chloro-3-methoxypyridazin-4-yl)-6-methylnicotinate (100.0 mg, 0.34 mmol) in 1,4-dioxane (1 mL) were sequentially added trimethylboroxine (51.0 mg, 0.41 mmol), cesium carbonate (333.0 mg, 1.02 mmol) and (1,1′-Bis(diphenylphosphino)ferrocene)dichloropalladium(II) (25.0 mg, 0.03 mmol) at 23° C. The resulting solution was stirred at 100° C. for 16 hr under nitrogen. The reaction mixture was diluted with ethyl acetate and filtered. The filtrate was collected and concentrated under vacuum. The resulting residue was dissolved in acetonitrile (2 mL) which was applied to a 40.0 g C18 column and purified by Combi Flash (Biotage Isolera Prime), eluted with 5˜28% acetonitrile in water within 30 min to afford methyl 4-(3-methoxy-6-methylpyridazin-4-yl)-6-methylnicotinate (75.0 mg, 79%) as a yellow oil. MS (ESI) calc'd for (C₁₄H₁₅N₃O3) (M+1)⁺, 274.1. found 274.1.

### Step-3: 4-(3-methoxy-6-methylpyridazin-4-yl)-6-methylnicotinic Acid

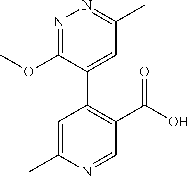 [SMILES: COc1nnc(C)cc1-c1cc(C)ncc1C(=O)O]

To a stirred solution of methyl 4-(3-methoxy-6-methylpyridazin-4-yl)-6-methylnicotinate (55.0 mg, 0.20 mmol) in methanol (0.9 mL) were added water (0.3 mL) and lithium hydroxide (19.0 mg, 0.81 mmol) at 23° C. The resulting solution was stirred at 23° C. for 0.5 hr under nitrogen. The resulting residue was acidified by citric acid which was applied to a 20.0 g C18 column and purified by Combi Flash (Biotage Isolera Prime), eluted with 5˜28% acetonitrile in water within 30 min to afford 4-(3-methoxy-6-methylpyridazin-4-yl)-6-methylnicotinic acid (47.0 mg, 89%) as a yellow solid. MS (ESI) calc'd for (C₁₃H₁₃N₃O₃) (M+1)⁺, 260.1. found 260.1.

### Step-4: N-(5-methoxy-1,3,4-thiadiazol-2-yl)-4-(3-methoxy-6-methylpyridazin-4-yl)-6-methylnicotinamide

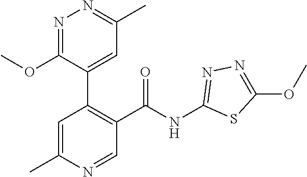

To a stirred solution of 4-(3-methoxy-6-methylpyridazin-4-yl)-6-methylnicotinic acid (47.0 mg, 0.18 mmol) and 5-methoxy-1,3,4-thiadiazol-2-amine (26.0 mg, 0.20 mmol) in acetonitrile (1 mL) was added 1-methylimidazole (74.6 mg, 0.91 mmol). Then TCFH (51.0 mg, 0.18 mmol) in acetonitrile (1 mL) was added to the above mixture at 23° C. The resulting solution was stirred at 23° C. for 2 hr under nitrogen. The resulting residue was dissolved in acetonitrile (1 mL) which was applied to a 20.0 g C18 column and purified by Combi Flash (Biotage Isolera Prime), eluted with 5˜34% acetonitrile in water within 30 min to afford N-(5-methoxy-1,3,4-thiadiazol-2-yl)-4-(3-methoxy-6-methylpyridazin-4-yl)-6-methylnicotinamide (31.0 mg, 45%) as a white solid. MS (ESI) calc'd for (C₁₆H₁₆N₆O₃S) (M+1)⁺, 373.1. found 373.2. ¹H NMR (400 MHz, DMSO-d₆) δ 13.01 (s, 1H), 8.86 (s, 1H), 7.62 (s, 1H), 7.46 (s, 1H), 4.08 (s, 3H), 3.74 (s, 3H), 2.60 (s, 6H).

### Example 44

### 2′-ethyl-5′-methoxy-N-(5-methoxy-1,3,4-thiadiazol-2-yl)-6-methyl-(4,4′-bipyridine)-3-carboxamide

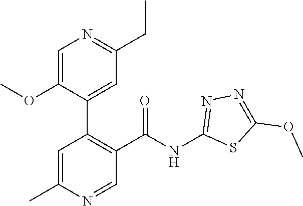

### Step-1: methyl 5′-methoxy-6-methyl-2′-vinyl-(4,4′-bipyridine)-3-carboxylate

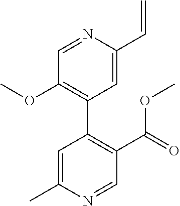

To a solution of methyl 2′-chloro-5′-methoxy-6-methyl-(4,4′-bipyridine)-3-carboxylate (400.0 mg, 1.33 mmol) in 1,4-Dioxane (8 mL) were added Pd(dppf)Cl₂ (87.0 mg, 0.13 mmol), K₂CO₃ (370.0 mg, 2.68 mmol) and trifluoro(vinyl)-14-borane potassium salt (179.0 mg, 1.33 mmol), water (2 mL) at 20° C. The resulting solution was stirred at 80° C. for 2 hr. The reaction mixture was quenched by the addition of water and extracted with ethyl acetate. The combined organic layers were washed with brine, dried over anhydrous sodium sulfate, filtered, and concentrated under vacuum. The residue was dissolved in DCM (5 mL) and purified by Combi Flash (Biotage Isolera Prime) which applied to a 40 g silica gel column that was eluted with 0˜26% ethyl acetate in petroleum ether within 35 min to afford methyl 5′-methoxy-6-methyl-2′-vinyl-(4,4′-bipyridine)-3-carboxylate (350.0 mg, 65%) as a red oil. MS (ESI) calc'd for (C₁₆H₁₆N₂O₃) (M+1)⁺, 285.1. found 285.1.

### Step-2: methyl 2′-ethyl-5′-methoxy-6-methyl-(4,4′-bipyridine)-3-carboxylate

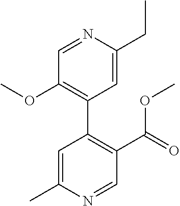

A mixture of methyl 5′-methoxy-6-methyl-2′-vinyl-(4,4′-bipyridine)-3-carboxylate (342.0 mg, 0.85 mmol) in Methanol (2 mL) was added Pd/C (dry, 50.0 mg) under hydrogen atmosphere. The resulting solution was stirred at 20° C. for 2 hr under hydrogen atmosphere. The suspension was filtered. The filtrate was collected and concentrated under vacuum. The residue was dissolved in DCM (3 mL) and purified by Combi Flash (Biotage Isolera Prime) which applied to a 40 g silica gel column that was eluted with 0˜7% methanol in dichloromethane within 35 min to afford methyl 2′-ethyl-5′-methoxy-6-methyl-(4,4′-bipyridine)-3-carboxylate (300.0 mg, 96%) as a yellow oil. MS (ESI) calc'd for (C₁₆H₁₈N₂O₃) (M+1)⁺, 287.1. found 287.1.

### Step-3: 2′-ethyl-5′-methoxy-6-methyl-(4,4′-bipyridine)-3-carboxylic Acid

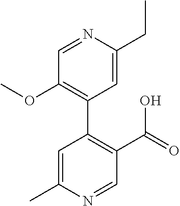 [SMILES: CCc1cc(-c2cc(C)ncc2C(=O)O)c(OC)cn1]

To a solution of methyl 2′-ethyl-5′-methoxy-6-methyl-(4,4′-bipyridine)-3-carboxylate (300.0 mg, 0.81 mmol) in Tetrahydrofuran (THF) (3 mL) were added LiOH (78.0 mg, 3.27 mmol) and Water (1.0 mL) at 17 ºC. The resulting solution was stirred at 50° C. for 2 hr. The reaction mixture was quenched by the addition of water and extracted with ethyl acetate. The combined organic layers were washed with brine, dried over anhydrous sodium sulfate, filtered, and concentrated under vacuum to afford 2′-ethyl-5′-methoxy-6-methyl-(4,4′-bipyridine)-3-carboxylic acid (246.0 mg, 88%) as a red oil. MS (ESI) calc'd for (C₁₅H₁₆N₂O₃) (M+1)⁺, 273.1. found 273.1.

### Step-4: 2′-ethyl-5′-methoxy-N-(5-methoxy-1,3,4-thiadiazol-2-yl)-6-methyl-(4,4′-bipyridine)-3-carboxamide

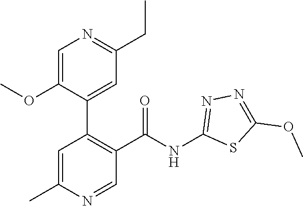

To a mixture of 5-methoxy-1,3,4-thiadiazol-2-amine (69.4 mg, 0.52 mmol) in Acetonitrile (2 mL) were added 2′-ethyl-5′-methoxy-6-methyl-(4,4′-bipyridine)-3-carboxylic acid (150.0 mg, 0.44 mmol) and 1-methyl-1H-imidazole (109.0 mg, 1.32 mmol). A solution of TCFH (185.0 mg, 0.66 mmol) in Acetonitrile (1 mL) was added thereto dropwise under nitrogen. The organic solvent was removed under vacuum. The resulting residue was dissolved in DMF (5 mL) which was applied to a 40 g C18 column and purified by Combi Flash (Biotage Isolera Prime), eluted with 5˜23% acetonitrile in water within 34 min to afford a white solid (70%). The product (70%) was dissolved in DMF (5 mL) and further purified by prep-HPLC with the following condition: (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 20% B to 40% B in 8 min, 40% B to 95% B in 8.2 min, 95% B to 95% B in 9.5 min, 95% B to 5% B in 11 min, 5% B; Wave Length: 254 nm; RT1 (min): 5.6; Injection Volume: 1.5 mL; Number Of Runs: 3) to afford 2′-ethyl-5′-methoxy-N-(5-methoxy-1,3,4-thiadiazol-2-yl)-6-methyl-(4,4′-bipyridine)-3-carboxamide (51.1 mg, 29%) as a white solid. MS (ESI) calc'd for (C₁₈H₁₉N₅O₃S) (M+1)⁺, 386.1. found 386.1. ¹H NMR (400 MHz, DMSO-d₆) δ 12.87 (s, 1H), 8.76 (s, 1H), 8.22 (s, 1H), 7.36 (s, 1H), 7.24 (s, 1H), 4.06 (s, 3H), 3.60 (s, 3H), 2.74-2.76 (m, 2H), 2.58 (s, 3H), 1.24 (t, J=7.6 Hz, 3H).

### Example 45 and 46

### 2′-chloro-N-(5-(((1s,3s)-3-hydroxycyclopentyl)oxy)-1,3,4-thiadiazol-2-yl)-5′-methoxy-6-methyl-(4,4′-bipyridine)-3-carboxamide, and 2′-chloro-N-(5-(((1s,3r)-3-hydroxycyclopentyl)oxy)-1,3,4-thiadiazol-2-yl)-5′-methoxy-6-methyl-(4,4′-bipyridine)-3-carboxamide

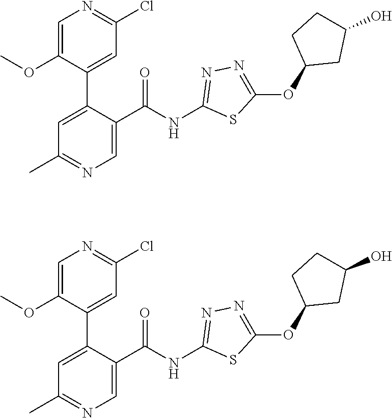 [SMILES: COc1cnc(Cl)cc1-c1cc(C)ncc1C(=O)Nc1nnc(O[C@H]2CC[C@H](O)C2)s1 ; COc1cnc(Cl)cc1-c1cc(C)ncc1C(=O)Nc1nnc(O[C@H]2CC[C@@H](O)C2)s1]

### Step 1 3-((tert-butyldimethylsilyl)oxy)cyclopentan-1-ol

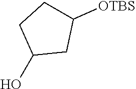 [SMILES: CC(C)(C)[Si](C)(C)OC1CCC(O)C1]

To a stirred solution of cyclopentane-1,3-diol (1.0 g, 9.79 mmol) in dichloromethane (5 mL) were sequentially added TBS-Cl (1.5 g, 9.79 mmol) and imidazole (0.7 g, 9.79 mmol) at 0 ºC. The resulting solution was stirred at 0° C. for 2 hr. The organic solvent was removed under vacuum. The resulting residue was dissolved in DCM (2 mL) and purified by Combi Flash (Biotage Isolera Prime) which applied to a 40 g silica gel column that was eluted with 0˜10% methanol in dichloromethane within 30 min to afford 3-((tert-butyldimethylsilyl)oxy)cyclopentan-1-ol (620.0 mg, 25%) as a colorless oil.

### Step 2 O-(3-((tert-butyldimethylsilyl)oxy)cyclopentyl) S-methyl Carbonodithioate

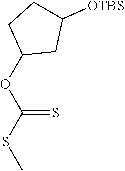 [SMILES: CSC(=S)OC1CCC(O[Si](C)(C)C(C)(C)C)C1]

To a degassed solution of 3-((tert-butyldimethylsilyl)oxy)cyclopentan-1-ol (600.0 mg, 2.77 mmol) in dry tetrahydrofuran (4 mL) was added NaH (222.0 mg, 5.55 mmol, 60%) in portions at 0° C. and stirred at 0° C. for 30 min under nitrogen atmosphere. Then CS₂ (0.3 mL, 4.16 mmol) was added to the above mixture at 0° C. and stirred at 0° C. for 20 min. To the above solution was added MeI (0.3 mL, 4.16 mmol) at 0° C. under nitrogen. The resulting mixture was then stirred at 0° C. for 1 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (2 mL) and purified by Combi Flash (Biotage Isolera Prime) which applied to a 40 g silica gel column that was eluted with 0˜30% ethyl acetate in petroleum ether within 20 min to afford O-(3-((tert-butyldimethylsilyl)oxy)cyclopentyl)S-methyl carbonodithioate (744.0 mg, 74%) as a yellow oil.

### Step 3 O-(3-((tert-butyldimethylsilyl)oxy)cyclopentyl) hydrazinecarbothioate

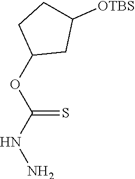 [SMILES: CC(C)(C)[Si](C)(C)OC1CCC(OC(=S)NN)C1]

To a stirred solution of O-(3-((tert-butyldimethylsilyl)oxy)cyclopentyl)S-methyl carbonodithioate (710.0 mg, 2.32 mmol) in methanol (5 mL) was added hydrazine (0.1 mL, 2.32 mmol) at 20° C. The resulting solution was stirred at 20° C. for 1 hr. The solvents were removed under vacuum to afford O-(3-((tert-butyldimethylsilyl)oxy)cyclopentyl) hydrazinecarbothioate (673.0 mg, crude) as a colorless oil. MS (ESI) calculated for (C₁₂H₂₆N₂O₂SSi) (M+1)⁺, 291.0. found, 291.3.

### Step 4 5-((3-((tert-butyldimethylsilyl)oxy)cyclopentyl)oxy)-1,3,4-thiadiazol-2-amine

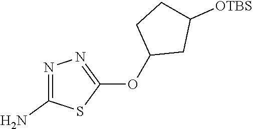 [SMILES: CC(C)(C)[Si](C)(C)OC1CCC(Oc2nnc(N)s2)C1]

To a stirred solution of O-(3-((tert-butyldimethylsilyl)oxy)cyclopentyl) hydrazinecarbothioate (670.0 mg, 2.31 mmol) in methanol (4 mL) were sequentially added TEA (0.7 mL, 4.61 mmol) and BrCN (366.0 mg, 3.46 mmol) at 20° C. The resulting solution was stirred at 20° C. for 2 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (4 mL) which was applied to a 40 g C18 column and purified by Combi Flash (Biotage Isolera Prime), eluted with 15˜80% acetonitrile in water within 30 min to afford 5-((3-((tert-butyldimethylsilyl)oxy)cyclopentyl)oxy)-1,3,4-thiadiazol-2-amine (301.0 mg, 34%) as a yellow solid. MS (ESI) calculated for (C₁₃H₂₅N₃O₂SSi) (M+1)⁺, 316.0. found, 316.3.

### Step 5 2-(5-((3-((tert-butyldimethylsilyl)oxy)cyclopentyl)oxy)-1,3,4-thiadiazol-2-yl)-1-(2′-chloro-5′-methoxy-6-methyl-(4,4′-bipyridine)-3-yl)ethan-1-one

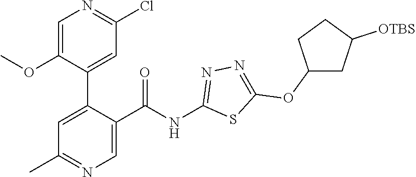 [SMILES: COc1cnc(Cl)cc1-c1cc(C)ncc1C(=O)Nc1nnc(OC2CCC(O[Si](C)(C)C(C)(C)C)C2)s1]

To a stirred solution of 5-((3-((tert-butyldimethylsilyl)oxy)cyclopentyl)oxy)-1,3,4-thiadiazol-2-amine (300.0 mg, 0.95 mmol) in acetonitrile (3 mL) were added Intermediate G (240.6 mg, 0.86 mmol) and 1-Methylimidazole (354.0 mg, 4.32 mmol) at 23 ºC. To the above solution was added a solution of TCFH (242.0 mg, 0.86 mmol) in acetonitrile (1 mL) at 23° C. under nitrogen. The resulting mixture was then stirred at 23° C. for 1 hr. The solvents were removed under vacuum. The resulting residue was dissolved in DMF (4 mL) which was applied to a 40 g C18 column and purified by Combi Flash (Biotage Isolera Prime), eluted with 5˜70% acetonitrile in water within 35 min to afford 2-(5-((3-((tert-butyldimethylsilyl)oxy)cyclopentyl)oxy)-1,3,4-thiadiazol-2-yl)-1-(2′-chloro-5′-methoxy-6-methyl-(4,4′-bipyridine)-3-yl)ethan-1-one (330.0 mg, 66%) as a white solid. MS (ESI) calculated for (C₂₆H₃₄ClN₅O₄SSi) (M+1)⁺, 576.0. found, 576.4.

### Step 6 2′-chloro-N-(5-(((1s,3s)-3-hydroxycyclopentyl)oxy)-1,3,4-thiadiazol-2-yl)-5′-methoxy-6-methyl-(4,4′-bipyridine)-3-carboxamide and 2′-chloro-N-(5-(((1s,3r)-3-hydroxycyclopentyl)oxy)-1,3,4-thiadiazol-2-yl)-5′-methoxy-6-methyl-(4,4′-bipyridine)-3-carboxamide

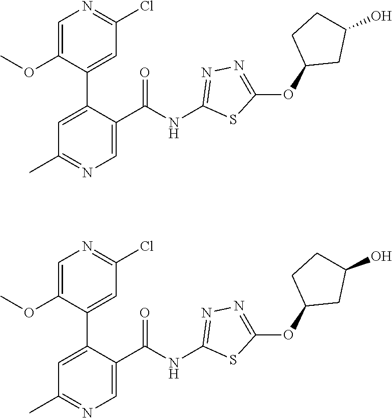 [SMILES: COc1cnc(Cl)cc1-c1cc(C)ncc1C(=O)Nc1nnc(O[C@H]2CC[C@H](O)C2)s1 ; COc1cnc(Cl)cc1-c1cc(C)ncc1C(=O)Nc1nnc(O[C@H]2CC[C@@H](O)C2)s1]

To a stirred solution of N-(5-((3-((tert-butyldimethylsilyl)oxy)cyclopentyl)oxy)-1,3,4-thiadiazol-2-yl)-2′-chloro-5′-methoxy-6-methyl-(4,4′-bipyridine)-3-carboxamide (330.0 mg, 0.57 mmol) in dichloromethane (4 mL) was added trifluoroacetic acid (0.8 mL) at 0° C. The resulting solution was stirred at 23° C. for 2 hr. The organic solvent was removed under vacuum. The mixture was diluted with water. The aqueous layer was basified with NaHCO₃ to pH 7˜8 and extracted with ethyl acetate. The combined organic layers were washed with brine, dried over anhydrous sodium sulfate, filtered, and concentrated under vacuum. The resulting residue was dissolved in DMF (6 mL) which was applied to a 80 g C18 column and purified by Combi Flash (Biotage Isolera Prime), eluted with 5˜80% acetonitrile in water within 30 min to afford a mixture of 2′-chloro-N-(5-((3-hydroxycyclopentyl)oxy)-1,3,4-thiadiazol-2-yl)-5′-methoxy-6-methyl-(4,4′-bipyridine)-3-carboxamide (160.0 mg, 59%) as a white solid. The mixture of compounds (160.0 mg) was separated by prep-chiral HPLC with the following condition: (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 20% B to 40% B in 8 min, 40% B; Wave Length: 254 nm; RT1 (min): 6.25) to afford 2′-chloro-N-(5-(((1s,3s)-3-hydroxycyclopentyl)oxy)-1,3,4-thiadiazol-2-yl)-5′-methoxy-6-methyl-(4,4′-bipyridine)-3-carboxamide, (9.0 mg, 6% yield) as a white solid with the first peak on chiral HPLC and 2′-chloro-N-(5-(((1s,3r)-3-hydroxycyclopentyl)oxy)-1,3,4-thiadiazol-2-yl)-5′-methoxy-6-methyl-(4,4′-bipyridine)-3-carboxamide, (70.2 mg, 43% yield) as a white solid with the second peak on chiral HPLC. The absolute stereochemistry was not determined and was arbitrarily assigned.

2′-chloro-N-(5-(((1s,3s)-3-hydroxycyclopentyl)oxy)-1,3,4-thiadiazol-2-yl)-5′-methoxy-6-methyl-(4,4′-bipyridine)-3-carboxamide: MS (ESI) calculated for (C₂₀H₂₀ClN₅O₄S) (M+1)⁺, 462.0. found, 462.1. ¹H NMR (400 MHz, DMSO-d₆) δ 12.92 (s, 1H), 8.83 (s, 1H), 8.15 (s, 1H), 7.49 (s, 1H), 7.37 (s, 1H), 5.25-5.11 (m, 1H), 4.68-4.53 (m, 1H), 4.27-4.09 (m, 1H), 3.63 (s, 3H), 2.67 (s, 3H), 2.35-2.28 (m, 1H), 2.02-1.76 (m, 2H), 1.74-1.73 (m, 3H). 2′-chloro-N-(5-(((1s,3r)-3-hydroxycyclopentyl)oxy)-1,3,4-thiadiazol-2-yl)-5′-methoxy-6-methyl-(4,4′-bipyridine)-3-carboxamide: MS (ESI) calculated for (C₂₀H₂₀ClN₅O₄S) (M+1)⁺, 462.0. found, 462.1. ¹H NMR (400 MHz, DMSO-d₆) δ 12.85 (s, 1H), 8.81 (s, 1H), 8.17 (s, 1H), 7.52 (s, 1H), 7.41 (s, 1H), 5.39-5.26 (m, 1H), 4.64-4.50 (m, 1H), 4.27-4.13 (m, 1H), 3.63 (s, 3H), 2.59 (s, 3H), 2.20-2.18 (m, 1H), 2.00-1.70 (m, 2H), 1.74-1.73 (m, 2H), 1.54-1.50 (m, 1H).

### Example 47

### 2′-chloro-3′-fluoro-5′-methoxy-N-(5-methoxy-1,3,4-thiadiazol-2-yl)-6-methyl-(4,4′-bipyridine)-3-carboxamide

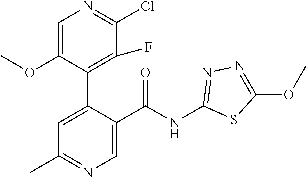

### Step-1: 2-chloro-3-fluoro-5-methoxypyridine

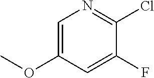

To a solution of 6-chloro-5-fluoropyridin-3-ol (20.0 g, 135.60 mmol) in Acetone (150 mL) were added MeI (17 mL, 271.00 mmol) and K₂CO₃ (37.5 g, 271.00 mmol) at 25° C. under nitrogen atmosphere. The resulting solution was stirred at 25° C. for 16 h under nitrogen before concentrated under vacuum. The reaction mixture was quenched with water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 330 g silica gel column and eluted with 0˜22% ethyl acetate in petroleum ether within 45 min to afford 2-chloro-3-fluoro-5-methoxypyridine (16.0 g, 80%) as a colorless oil. MS (ESI) calc'd for (C₆H₅ClFNO) (M+1)⁺, 162.0. found 162.0.

### Step-2: 2-chloro-3-fluoro-4-iodo-5-methoxypyridine

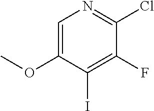

To a degassed solution of 2-chloro-3-fluoro-5-methoxypyridine (16.0 g, 99.00 mmol) in dry

Tetrahydrofuran (160 mL) was added n-butyllithium (44 mL, 110.00 mmol, 2.5 N in hexane) dropwise at −60° C. and stirred at −60° C. for 1 hr under nitrogen atmosphere. Then iodine (27.6 g, 109.00 mmol) was added to the above mixture at −60° C. The resulting solution was stirred at −60˜20° C. for 2 hr. The reaction mixture was quenched by the addition of saturated sodium thiosulfate aqueous solution and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 330 g silica gel column and eluted with 0˜50% ethyl acetate in petroleum ether within 40 min to afford 2-chloro-3-fluoro-4-iodo-5-methoxypyridine (22.0 g, 73%) as a white solid MS (ESI) calc'd for (C₆H₄ClFINO) (M+1)⁺, 287.9. found, 287.9.

### Step-3: methyl 2′-chloro-3′-fluoro-5′-methoxy-6-methyl-(4,4′-bipyridine)-3-carboxylate

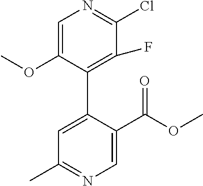

To a degassed solution of methyl 6-methyl-4-(4,4,5,5-tetramethyl-1,3,2-dioxaborolan-2-yl)nicotinate (7.2 g, 26.10 mmol) and 2-chloro-3-fluoro-4-iodo-5-methoxypyridine (5.0 g, 17.39 mmol) in dry 1,4-Dioxane (50 mL) were added Water (10 mL), (1,1′-Bis(diphenylphosphino)ferrocene)dichloropalladium(II), complex with dichloromethane (4.2 g, 5.15 mmol) and K₂CO₃ (7.2 g, 52.20 mmol) at 25° C. under nitrogen atmosphere. The resulting solution was stirred at 25° C. for 2 h under nitrogen atmosphere. The suspension was filtered. The filtrate was collected and concentrated under vacuum. The resulting residue was purified by Combi Flash (Biotage Isolera Prime) which applied to 120 g silica gel column and eluted with 0˜46% ethyl acetate in petroleum ether within 45 min to afford methyl 2′-chloro-3′-fluoro-5′-methoxy-6-methyl-(4,4′-bipyridine)-3-carboxylate (2.8 g, 53%) as a white solid. MS (ESI) calc'd for (C₁₄H₁₂ClFN₂O₃) (M+1)⁺, 311.1. found, 311.1.

### Step-4: 2′-chloro-3′-fluoro-5′-methoxy-6-methyl-(4,4′-bipyridine)-3-carboxylic Acid

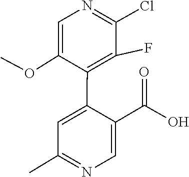 [SMILES: COc1cnc(Cl)c(F)c1-c1cc(C)ncc1C(=O)O]

To a stirred solution of methyl 2′-chloro-3′-fluoro-5′-methoxy-6-methyl-(4,4′-bipyridine)-3-carboxylate (2.8 g, 9.17 mmol) in Methanol (10 mL) were added NaOH (1.4 g, 36.70 mmol) and Water (10 mL) at 25° C. The resulting solution was stirred at 25° C. for 2 h before diluted with water. The organic solvent was removed under vacuum. The aqueous layer was acidified with Citric acid to pH ˜5 and extracted with ethyl acetate. The combined organic layers were washed with brine, dried over anhydrous sodium sulfate, filtered, and concentrated under vacuum to afford 2′-chloro-3′-fluoro-5′-methoxy-6-methyl-(4,4′-bipyridine)-3-carboxylic acid (1.3 g, crude) as a yellow oil. MS (ESI) calc'd for (C₁₃H₁₀ClFN₂O₃) (M+1)⁺, 297.0. found 297.0.

### Step-5: 2′-chloro-3′-fluoro-5′-methoxy-N-(5-methoxy-1,3,4-thiadiazol-2-yl)-6-methyl-(4,4′-bipyridine)-3-carboxamide

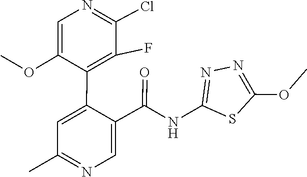

To a solution of 2′-chloro-3′-fluoro-5′-methoxy-6-methyl-(4,4′-bipyridine)-3-carboxylic acid (130.0 mg, 0.43 mmol) in dry acetonitrile (1 mL) were added 5-methoxy-1,3,4-thiadiazol-2-amine (57.0 mg, 0.43 mmol) and 1-methyl-1H-imidazole (180.0 mg, 2.15 mmol) at 25° C. Then a solution of TCFH (123 mg, 0.44 mmol) in acetonitrile was added to the above mixture at 25° C. The resulting solution was stirred at 25° C. for 2 hr. The mixture was applied to a 20 g C18 column and purified by Combi Flash (Biotage Isolera Prime), eluted with 5˜45% acetonitrile in water within 40 min to afford 2′-chloro-3′-fluoro-5′-methoxy-N-(5-methoxy-1,3,4-thiadiazol-2-yl)-6-methyl-(4,4′-bipyridine)-3-carboxamide (21.4 mg, 11%) as a white solid. MS (ESI) calc'd for (C₁₆H₁₃ClF₂N₂O₃S) (M+1)⁺, 410.0. found 410.0. ¹H NMR (400 MHz, DMSO-d₆) δ 13.04 (s, 1H), 8.98 (s, 1H), 8.18 (s, 1H), 7.47 (s, 1H), 4.07 (s, 3H), 3.74 (s, 3H), 2.60 (s, 3H).

### Example 48

### 2′-chloro-5′-(difluoromethoxy)-N-(5-methoxy-1,3,4-thiadiazol-2-yl)-6-methyl-(4,4′-bipyridine)-3-carboxamide

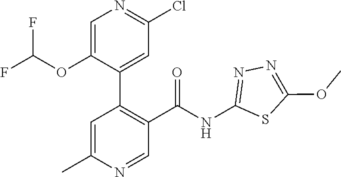 [SMILES: COc1nnc(NC(=O)c2cnc(C)cc2-c2cc(Cl)ncc2OC(F)F)s1]

### Step-1: 2-chloro-5-(difluoromethoxy)-4-iodopyridine

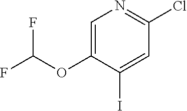 [SMILES: FC(F)Oc1cnc(Cl)cc1I]

To a stirred solution of 6-chloro-4-iodopyridin-3-ol (10.0 g, 39.14 mmol) in N,N-Dimethylformamide (DMF) (50 mL) were added sodium 2-chloro-2,2-difluoroacetate (11.9 g, 78.20 mmol) and Cs₂CO₃ (16.6 g, 50.80 mmol) at 25° C. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to a 330 g silica gel column that was eluted with 0˜25% ethyl acetate in petroleum ether within 40 min to afford 2-chloro-5-(difluoromethoxy)-4-iodopyridine (10.5 g, 79%) as a white solid. MS (ESI) calc'd for (C₆H₃ClF₂INO) (M+1)⁺, 305.4. found 305.4.

### Step-2: methyl 2′-chloro-5′-(difluoromethoxy)-6-methyl-(4,4′-bipyridine)-3-carboxylate

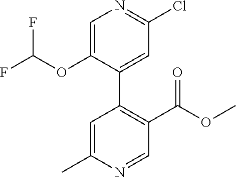 [SMILES: COC(=O)c1cnc(C)cc1-c1cc(Cl)ncc1OC(F)F]

To a degassed solution of methyl 6-methyl-4-(4,4,5,5-tetramethyl-1,3,2-dioxaborolan-2-yl)nicotinate (5.0 g, 18.04 mmol) in 1,4-Dioxane (40 mL) were added 2-chloro-5-(difluoromethoxy)-4-iodopyridine (5.5 g, 18.04 mmol), Water (8 mL), K₂CO₃ (7.5 g, 54.1 mmol) and (1,1′-Bis(diphenylphosphino)ferrocene)dichloropalladium(II) complex with dichloromethane (4.4 g, 5.41 mmol) at 23° C. under nitrogen atmosphere. The resulting solution was stirred at 80° C. for 2 h under nitrogen atmosphere. The suspension was filtered. The filtrate was concentrated under vacuum. The resulting residue was purified by Combi Flash (Biotage Isolera Prime) which applied to 120 g silica gel column and eluted with 0˜56% ethyl acetate in petroleum ether within 40 min to afford methyl 2′-chloro-5′-(difluoromethoxy)-6-methyl-(4,4′-bipyridine)-3-carboxylate (5.7 g, 63%) as a white solid. MS (ESI) calc'd for (C₁₄H₁₁ClF₂N₂O₃) (M+1)⁺, 329.0. found, 329.0.

### Step-3: 2′-chloro-5′-(difluoromethoxy)-6-methyl-(4,4′-bipyridine)-3-carboxylic Acid

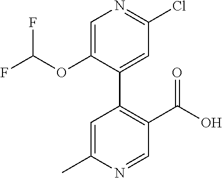 [SMILES: Cc1cc(-c2cc(Cl)ncc2OC(F)F)c(C(=O)O)cn1]

To a stirred solution of methyl 2′-chloro-5′-(difluoromethoxy)-6-methyl-(4,4′-bipyridine)-3-carboxylate (3.0 g, 9.13 mmol) in Methanol (20 mL) were added NaOH (1.4 g, 36.50 mmol) and Water (20 mL) at 25° C. The resulting solution was stirred at 25° C. for 1 h before diluted with water. The organic solvent was removed under vacuum. The aqueous layer was acidified with Citric acid to pH ˜6 and extracted with ethyl acetate. The combined organic layers were washed with brine, dried over anhydrous sodium sulfate, filtered, and concentrated under vacuum to afford 2′-chloro-5′-(difluoromethoxy)-6-methyl-(4,4′-bipyridine)-3-carboxylic acid (2.5 g, crude) as a yellow oil. MS (ESI) calc'd for (C₁₃H₉ClF₂N₂O₃) (M+1)⁺, 315.0. found 315.0.

### Step-4: 2′-chloro-5′-(difluoromethoxy)-N-(5-methoxy-1,3,4-thiadiazol-2-yl)-6-methyl-(4,4′-bipyridine)-3-carboxamide

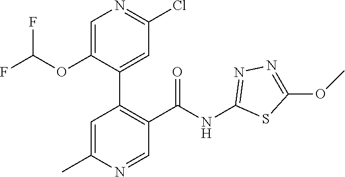 [SMILES: COc1nnc(NC(=O)c2cnc(C)cc2-c2cc(Cl)ncc2OC(F)F)s1]

To a stirred solution of 2′-chloro-5′-(difluoromethoxy)-6-methyl-(4,4′-bipyridine)-3-carboxylic acid (50.0 mg, 0.15 mmol) in Acetonitrile (1 mL) were added 5-methoxy-1,3,4-thiadiazol-2-amine (22.0 mg, 0.17 mmol) and 1-methyl-1H-imidazole (65.0 mg, 0.79 mmol) at 25° C. Then a solution of TCFH (44.0 mg, 0.15 mmol) in acetonitrile (0.5 mL) was added to the above mixture at 25° C. The resulting solution was stirred at 25° C. for 1 hr. The resulting mixture was applied to a 20 g C18 column and purified by Combi Flash (Biotage Isolera Prime), eluted with 5˜45% acetonitrile in water within 40 min to afford 2′-chloro-5′-(difluoromethoxy)-N-(5-methoxy-1,3,4-thiadiazol-2-yl)-6-methyl-(4,4′-bipyridine)-3-carboxamide (27.5 mg, 38% yield) as a white solid. MS (ESI) calc'd for (C₁₆H₁₂ClF₂N₅O₃S) (M+1)⁺, 428.0. found 428.1. ¹H NMR (400 MHz, DMSO-d₆) δ 13.08 (s, 1H), 8.97 (s, 1H), 8.33 (s, 1H), 7.72 (s, 1H), 7.45 (s, 1H), 7.31-6.80 (m, 1H), 4.07 (s, 3H), 2.61 (s, 3H).

### Example 49

### 2′-chloro-5′-ethyl-N-(5-methoxy-1,3,4-thiadiazol-2-yl)-6-methyl-(4,4′-bipyridine)-3-carboxamide

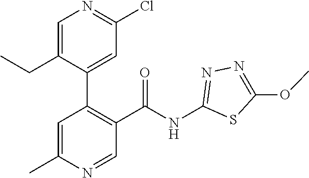 [SMILES: CCc1cnc(Cl)cc1-c1cc(C)ncc1C(=O)Nc1nnc(OC)s1]

### Step-1: methyl 5′-bromo-2′-chloro-6-methyl-(4,4′-bipyridine)-3-carboxylate

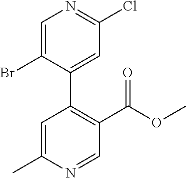 [SMILES: COC(=O)c1cnc(C)cc1-c1cc(Cl)ncc1Br]

To a degassed solution of methyl 4-chloro-6-methylnicotinate (1.0 g, 5.39 mmol) in 1,4-Dioxane (10 mL) were sequentially added Bis(pinacolato)diboron (2.7 g, 10.78 mmol), potassium acetate (1.6 g, 16.16 mmol) and (1,1′-Bis(diphenylphosphino)ferrocene)dichloropalladium(II) (1.2 g, 1.62 mmol) at 25° C. under nitrogen atmosphere. The resulting solution was stirred at ° C. for 3 hours under nitrogen. To the above mixture were added a solution of 5-bromo-2-chloro-4-iodopyridine (1.0 g, 3.61 mmol), water (2 mL), potassium carbonate (1.5 g, 10.83 mmol) and (1,1′-Bis(diphenylphosphino)ferrocene)dichloropalladium(II) (0792.0 mg, 1.08 mmol) in 1,4-Dioxane (10 mL) at 25° C. under nitrogen atmosphere. The resulting solution was stirred at 80° C. for 2 hr under nitrogen. The suspension was filtered. The filtrate was collected and concentrated under vacuum. The resulting residue was purified by Combi Flash (Biotage Isolera Prime) which applied to 80 g silica gel column and eluted with 0˜10% methanol in dichloromethane within 40 min to afford methyl 5′-bromo-2′-chloro-6-methyl-(4,4′-bipyridine)-3-carboxylate (2.1 g, 99%) as a brown oil. MS (ESI) calculated for (C₁₃H₁₀BrClN₂O₂) (M+1)⁺, 341.0. found, 341.0.

### Step-2: methyl 2′-chloro-6-methyl-5′-vinyl-(4,4′-bipyridine)-3-carboxylate

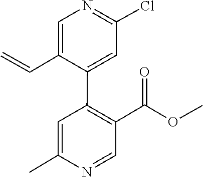 [SMILES: C=Cc1cnc(Cl)cc1-c1cc(C)ncc1C(=O)OC]

To a stirred solution of methyl 5′-bromo-2′-chloro-6-methyl-(4,4′-bipyridine)-3-carboxylate (400.0 mg, 1.17 mmol) and 4,4,5,5-tetramethyl-2-vinyl-1,3,2-dioxaborolane (180.0 mg, 1.17 mmol) in Toluene (2 mL) was added 1, l′-Bis (di-t-butylphosphino)ferrocene palladium dichloride (76.0 mg, 0.12 mmol) at 20° C. under nitrogen atmosphere. To the above solution was added potassium phosphate (994.0 mg, 4.68 mmol) in water (0.2 mL) at 50° C. The resulting solution was stirred at 100° C. for 16 hr under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (4 mL) and purified by Combi Flash (Biotage Isolera Prime) which applied to a 40 g silica gel column that was eluted with 0˜40% ethyl acetate in petroleum ether within 30 min to afford methyl 2′-chloro-6-methyl-5′-vinyl-(4,4′-bipyridine)-3-carboxylate (260.0 mg, 67%) as a brown oil. MS (ESI) calculated for (C₁₅H₁₃ClN₂O₂) (M+1)⁺, 289.1. found, 289.1.

### Step-3: methyl 2′-chloro-5′-ethyl-6-methyl-(4,4′-bipyridine)-3-carboxylate

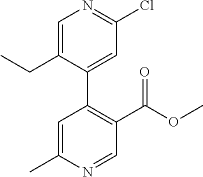 [SMILES: CCc1cnc(Cl)cc1-c1cc(C)ncc1C(=O)OC]

To a stirred solution of methyl 2′-chloro-6-methyl-5′-vinyl-(4,4′-bipyridine)-3-carboxylate (250.0 mg, 0.87 mmol) in Ethyl acetate (10 mL) was added platinum(IV) oxide (25.0 mg, 0.11 mmol) at 20° C. The resulting solution was stirred at 20° C. for 1 hour under hydrogen atmosphere. The suspension was filtered. The filtrate was collected and concentrated under vacuum to afford methyl 2′-chloro-5′-ethyl-6-methyl-(4,4′-bipyridine)-3-carboxylate (240.0 mg, crude) as a brown oil. MS (ESI) calculated for (C₁₅H₁₅ClN₂O₂) (M+1)⁺, 291.1. found, 291.1.

### Step-4: 2′-chloro-5′-ethyl-6-methyl-(4,4′-bipyridine)-3-carboxylic Acid

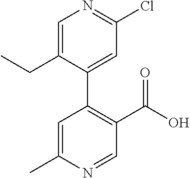 [SMILES: CCc1cnc(Cl)cc1-c1cc(C)ncc1C(=O)O]

To a stirred solution of methyl 2′-chloro-5′-ethyl-6-methyl-(4,4′-bipyridine)-3-carboxylate (240.0 mg, 0.83 mmol) in Methanol (3 mL) was added a solution of sodium hydroxide (132.0 mg, 3.30 mmol) in water (3 mL) at 20° C. The resulting solution was stirred at 80° C. for 1 hr. The organic solvent was removed under vacuum. The aqueous layer was acidified with citric acid to pH 5˜6 and extracted with ethyl acetate. The combined organic layers were washed with brine, dried over anhydrous sodium sulfate, filtered, and concentrated under vacuum to afford 2′-chloro-5′-ethyl-6-methyl-(4,4′-bipyridine)-3-carboxylic acid (190.0 mg, crude) as a white solid. MS (ESI) calculated for (C₁₄H₁₃ClN₂O₂) (M+1)⁺, 277.1. found, 277.1.

### Step-5: 2′-chloro-5′-ethyl-N-(5-methoxy-1,3,4-thiadiazol-2-yl)-6-methyl-(4,4′-bipyridine)-3-carboxamide

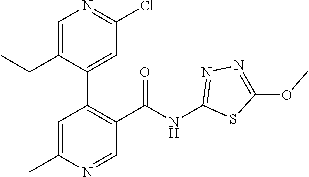 [SMILES: CCc1cnc(Cl)cc1-c1cc(C)ncc1C(=O)Nc1nnc(OC)s1]

To a stirred solution of 2′-chloro-5′-ethyl-6-methyl-(4,4′-bipyridine)-3-carboxylic acid (210.0 mg, 0.76 mmol) and 5-methoxy-1,3,4-thiadiazol-2-amine (100.0 mg, 0.76 mmol) in acetonitrile (1 mL) was added 1-methyl-1H-imidazole (312.0 mg, 3.79 mmol). To the above solution was added a solution of TCFH (213.0 mg, 0.76 mmol) in acetonitrile (1 mL) at 20° C. under nitrogen. The resulting solution was stirred at 20° C. for 2 hr. The resulting residue was dissolved in DMF (3 mL) which was applied to a 40 g C18 column and purified by Combi Flash (Biotage Isolera Prime), eluted with 5˜60% acetonitrile in water within 30 min to afford a yellow solid (90% purity). The yellow solid (90% purity) was dissolved in DMF (2 mL) and purified by prep-HPLC with the following condition: (Column: Atlantis HILIC OBD Column, 19*150 mm*5 um; Mobile Phase A: Water (0.1% FA), Mobile Phase B: MeOH—HPLC; Flow rate: 25 mL/min; Gradient: 65% B to 65% B in 8 min, 65% B to 95% B in 8.2 min, 95% B to 95% B in 10 min, 95% B to 65% B in 11 min, 65% B; Wave Length: 220\254 nm; RT1 (min): 7; Injection Volume: 0.4 mL; Number Of Runs: 5) to afford 2′-chloro-5′-ethyl-N-(5-methoxy-1,3,4-thiadiazol-2-yl)-6-methyl-(4,4′-bipyridine)-3-carboxamide (64.5 mg, 21%) as a white solid. MS (ESI) calculated for (C₁₇H₁₆ClN₅O₂S) (M+1)⁺, 390.1. found, 390.0. ¹H NMR (400 MHz, DMSO-d₆) δ 13.04 (s, 1H), 8.97 (s, 1H), 8.36 (s, 1H), 7.33 (s, 1H), 7.29 (s, 1H), 4.06 (s, 3H), 2.60 (s, 3H), 2.41-2.29 (m, 2H), 1.00-0.92 (m, 3H).

### Example 50

### 7-(2-fluoro-6-methoxyphenyl)-N-(5-methoxy-1,3,4-thiadiazol-2-yl)imidazo(1,2-a)pyridine-6-carboxamide

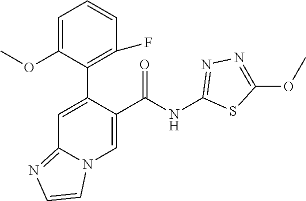

### Step-1: methyl 7-chloroimidazo(1,2-a)pyridine-6-carboxylate

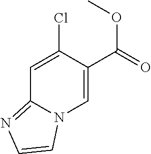

To a solution of methyl 6-amino-4-chloronicotinate (900.0 mg, 4.82 mmol) in Ethanol (6 mL) were added Sodium bicarbonate (689.0 mg, 8.20 mmol), 2-chloroacetaldehyde (4.3 g, 40% in water, 21.70 mmol) at 20° C. The resulting solution was stirred at 80° C. under nitrogen for 2 hr. The reaction mixture was quenched by the addition of water and extracted with ethyl acetate. The combined organic layers were washed with brine, dried over anhydrous sodium sulfate, filtered, and concentrated under vacuum. The resulting mixture was dissolved in DCM (6 mL) and purified by Combi Flash (Biotage Isolera Prime) which applied to an 80 g silica gel column that was eluted with 0˜10% methanol in dichloromethane within 35 min to afford methyl 7-chloroimidazo(1,2-a)pyridine-6-carboxylate (900.0 mg, 70%). MS (ESI) calc'd for (C₉H₇ClN₂O₂) (M+1)⁺, 211.0. found 211.0.

### Step-2: methyl 7-(2-fluoro-6-methoxyphenyl)imidazo(1,2-a)pyridine-6-carboxylate

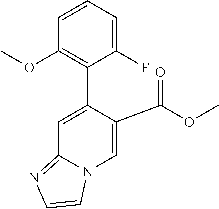

A mixture of methyl 7-chloroimidazo(1,2-a)pyridine-6-carboxylate (600.0 mg, 2.56 mmol), (2-fluoro-6-methoxyphenyl)boronic acid (654.0 mg, 3.85 mmol), XPhos (146.0 mg, 0.31 mmol) and XPhos Pd G3 (217.0 mg, 0.26 mmol) in 1,4-Dioxane (1 mL) and Water (0.3 mL) was stirred at 80° C. for 2 hr under nitrogen. The reaction mixture was diluted with water and extracted with ethyl acetate. The combined organic layers were washed with brine, dried over anhydrous sodium sulfate, filtered, and concentrated under vacuum. The resulting mixture was dissolved in DMF (5 mL) which was applied to a 40 g C18 column and purified by Combi Flash (Biotage Isolera Prime), eluted with 5˜35% acetonitrile in water within 40 min to afford methyl 7-(2-fluoro-6-methoxyphenyl)imidazo(1,2-a)pyridine-6-carboxylate (200.0 mg, 23%) as a white solid. MS (ESI) calc'd for (C₁₆H₁₃FN₂O₃) (M+1)⁺, 301.0. found 301.0.

### Step-3: 7-(2-fluoro-6-methoxyphenyl)imidazo(1,2-a)pyridine-6-carboxylic Acid

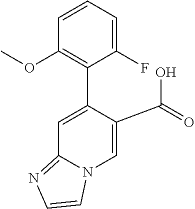 [SMILES: COc1cccc(F)c1-c1cc2nccn2cc1C(=O)O]

A mixture of methyl 7-(2-fluoro-6-methoxyphenyl)imidazo(1,2-a)pyridine-6-carboxylate (120.0 mg, 0.36 mmol) and LiOH (34.5 mg, 1.43 mmol) in Tetrahydrofuran (3 mL) and Water (1 mL) was stirred at 50° C. for 12 hr. The volatiles were removed under vacuum. The aqueous layer was acidified with citric acid to pH ˜5. The solvents were removed under vacuum to afford 7-(2-fluoro-6-methoxyphenyl)imidazo(1,2-a)pyridine-6-carboxylic acid (950.0 mg, crude) as a yellow solid, which was used directly without further purification. MS (ESI) calc'd for (C₁₅H₁₁FN₂O₃) (M+1)⁺, 287.0. found 286.9.

### Step-4: 7-(2-fluoro-6-methoxyphenyl)-N-(5-methoxy-1,3,4-thiadiazol-2-yl)imidazo(1,2-a)pyridine-6-carboxamide

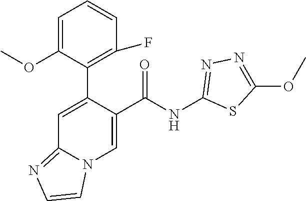

To a mixture of 7-(2-fluoro-6-methoxyphenyl)imidazo(1,2-a)pyridine-6-carboxylic acid (700.0 mg, 0.24 mmol) in Acetonitrile (3 mL) were added 5-methoxy-1,3,4-thiadiazol-2-amine (38.5 mg, 0.29 mmol), and NMI (120.0 mg, 1.46 mmol). To the above solution was added a solution of TCFH (103.0 mg, 0.36 mmol) in Acetonitrile (1 mL) at 20° C. under nitrogen. The mixture was stirred at 20° C. for 2 hr under nitrogen. The volatiles were removed under vacuum. The residue was dissolved in DMF (3 mL) which was applied to a 40 g C18 column and purified by Combi Flash (Biotage Isolera Prime), eluted with 5˜30% acetonitrile in water within 40 min to afford 7-(2-fluoro-6-methoxyphenyl)-N-(5-methoxy-1,3,4-thiadiazol-2-yl)imidazo(1,2-a)pyridine-6-carboxamide (30.9 mg, 30%) as a yellow solid. MS (ESI) calc'd for (C₁₈H₁₄FN₅O₃S) (M+1)⁺, 400.0. found 400.0. ¹H NMR (400 MHz, DMSO-d₆) δ 12.82 (s, 1H), 9.13 (s, 1H), 8.12 (d, J=1.2 Hz, 1H), 7.73 (d, J=1.2 Hz, 1H), 7.54 (s, 1H), 7.45-7.32 (m, 1H), 6.97-6.75 (m, 2H), 4.07 (s, 3H), 3.59 (s, 3H).

### Example 51

### 2′-cyclopropyl-5′-methoxy-N-(5-methoxy-1,3,4-thiadiazol-2-yl)-6-methyl-(4,4′-bipyridine)-3-carboxamide

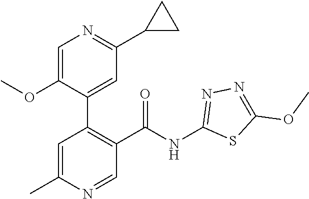

### Step-1: methyl 2′-cyclopropyl-5′-methoxy-6-methyl-(4,4′-bipyridine)-3-carboxylate

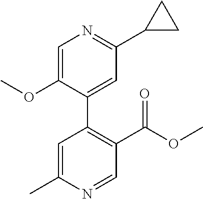

To a stirred solution of methyl 2′-chloro-5′-methoxy-6-methyl-(4,4′-bipyridine)-3-carboxylate (800.0 mg, 2.73 mmol) in 1,4-Dioxane (10 mL) were added cyclopropylboronic acid (235.0 mg, 2.73 mmol) and K₃PO₄ (1.2 g, 5.47 mmol) at 20° C. To the above solution was added PdAMphos (230.1 mg, 0.27 mmol) at 20° C. under nitrogen. The resulting mixture was then stirred at 80° C. for 2 hr under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (5 mL) and purified by Combi Flash (Biotage Isolera Prime) which applied to a 40 g silica gel column that was eluted with 0˜70% ethyl acetate in petroleum ether within 25 min to afford methyl 2′-cyclopropyl-5′-methoxy-6-methyl-(4,4′-bipyridine)-3-carboxylate (300.0 mg, 33%) as a yellow oil. MS (ESI) calc'd for (C₁₇H₁₈N₂O₃) (M+1)⁺, 299.2. found, 299.2.

### Step-2: 2′-cyclopropyl-5′-methoxy-6-methyl-(4,4′-bipyridine)-3-carboxylic Acid

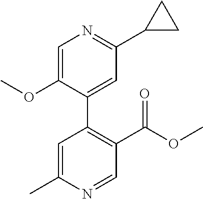

To a stirred solution of methyl 2′-cyclopropyl-5′-methoxy-6-methyl-(4,4′-bipyridine)-3-carboxylate (300.0 mg, 1.01 mmol) in Tetrahydrofuran (2 mL) was added a solution of lithium hydroxide (24.1 mg, 1.01 mmol) in Water (2 mL) at 20° C. The resulting solution was stirred at 20° C. for 1 hr. The organic solvent was removed under vacuum. The aqueous layer was acidified with citric acid to pH ˜5 and extracted with ethyl acetate. The combined organic layers were washed with brine, dried over anhydrous sodium sulfate, filtered, and concentrated under vacuum to afford 2′-cyclopropyl-5′-methoxy-6-methyl-(4,4′-bipyridine)-3-carboxylic acid (111.0 mg, crude) as a yellow solid. MS (ESI) calc'd for (C₁₆H₁₆N₂O₃) (M+1)+, 245.1. found, 245.1.

### Step-3: 2′-cyclopropyl-5′-methoxy-N-(5-methoxy-1,3,4-thiadiazol-2-yl)-6-methyl-(4,4′-bipyridine)-3-carboxamide

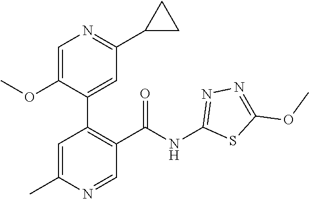

To a solution of 2′-cyclopropyl-5′-methoxy-6-methyl-(4,4′-bipyridine)-3-carboxylic acid (100 mg, 0.35 mmol) in Acetonitrile (2 mL) were added 5-methoxy-1,3,4-thiadiazol-2-amine (46.1 mg, 0.35 mmol) and 1-methylimidazole (144.3 mg, 1.76 mmol) at 20° C. under nitrogen. To the above solution was added a solution of TCFH (98.3 mg, 0.35 mmol) in Acetonitrile (2 mL) at 20° C. under nitrogen. The resulting mixture was then stirred at 20° C. for 1 hr. The resulting residue was dissolved in DMF (1 mL) which was applied to a 20 g C18 column and purified by Combi Flash (Biotage Isolera Prime), eluted with 5˜55% acetonitrile in water within 30 min to afford 2′-cyclopropyl-5′-methoxy-N-(5-methoxy-1,3,4-thiadiazol-2-yl)-6-methyl-(4,4′-bipyridine)-3-carboxamide (82.4 mg, 58%) as a white solid. MS (ESI) calc'd for (C₁₉H₁₉N₅O₃S) (M+1)⁺, 398.1. found 398.1. ¹H NMR (400 MHz, DMSO-d₆) δ 12.84 (s, 1H), 8.75 (s, 1H), 8.15 (s, 1H), 7.39 (s, 1H), 7.28 (s, 1H), 4.07 (s, 3H), 3.56 (s, 3H), 2.59 (s, 3H), 2.13-2.16 (m, 1H), 0.98-0.85 (m, 4H).

### Example 52

### 5′-(difluoromethoxy)-N-(5-methoxy-1,3,4-thiadiazol-2-yl)-2′,6-dimethyl-(4,4′-bipyridine)-3-carboxamide

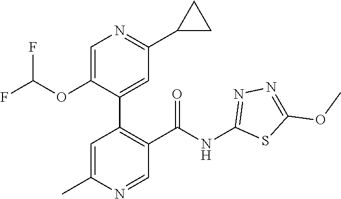 [SMILES: COc1nnc(NC(=O)c2cnc(C)cc2-c2cc(C3CC3)ncc2OC(F)F)s1]

### Step 1 methyl 5′-(difluoromethoxy)-2′,6-dimethyl-(4,4′-bipyridine)-3-carboxylate

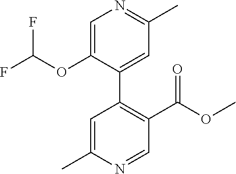 [SMILES: COC(=O)c1cnc(C)cc1-c1cc(C)ncc1OC(F)F]

To a degassed solution of methyl 2′-chloro-5′-(difluoromethoxy)-6-methyl-(4,4′-bipyridine)-3-carboxylate (150.0 mg, 0.45 mmol) in 1,2-Dimethoxyethane (2 mL) were added (1,1′-Bis(diphenylphosphino)ferrocene)dichloropalladium(II), K₂CO₃ (186.3 mg, 1.35 mmol) at 25° C. under nitrogen atmosphere. Then 2,4,6-trimethyl-1,3,5,2,4,6-trioxatriborinane (63 mg, 0.50 mmol) was added to the above mixture at 120° C. The resulting mixture was stirred at 120° C. for 3 hr. The suspension was filtered. The filtrate was concentrated under vacuum. The resulting residue was dissolved in acetonitrile (4 mL) which was applied to a 40 g C18 column and purified by Combi Flash (Biotage Isolera Prime), eluted with 5˜55% acetonitrile in water within 40 min to afford methyl 5′-(difluoromethoxy)-2′,6-dimethyl-(4,4′-bipyridine)-3-carboxylate (70 mg, 42%) as a yellow oil MS (ESI) calc'd for (C₁₅H₁₄F₂N₂O₃) (M+1)⁺, 309.1. found 309.1.

### Step 2 5′-(difluoromethoxy)-2′,6-dimethyl-(4,4′-bipyridine)-3-carboxylic Acid

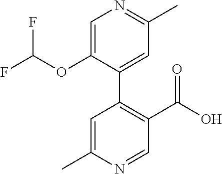 [SMILES: Cc1cc(-c2cc(C)ncc2C(=O)O)c(OC(F)F)cn1]

To a stirred solution of methyl 5′-(difluoromethoxy)-2′,6-dimethyl-(4,4′-bipyridine)-3-carboxylate (70.0 mg, 0.227 mmol) in Methanol (1 mL) were added NaOH (36.0 mg, 0.91 mmol) and Water (1 mL) at 25° C. The resulting solution was stirred at 25° C. for 2 hr. The organic solvent was removed under vacuum. The aqueous layer was acidified with Citric acid to pH ˜7 and extracted with ethyl acetate. The combined organic layers were washed with brine, dried over anhydrous sodium sulfate, filtered, and concentrated under vacuum. The resulting residue was dissolved in acetonitrile (2 mL) which was applied to a 20 g C18 column and purified by Combi Flash (Biotage Isolera Prime), eluted with 5˜30% acetonitrile in water within 30 min to afford 5′-(difluoromethoxy)-2′,6-dimethyl-(4,4′-bipyridine)-3-carboxylic acid (60.0 mg, 89%) as a yellow oil. MS (ESI) calc'd for (C₁₄H₁₂F₂N₂O₃) (M+1)⁺, 295.1. found, 295.1.

### Step 3 5′-(difluoromethoxy)-N-(5-methoxy-1,3,4-thiadiazol-2-yl)-2′,6-dimethyl-(4,4′-bipyridine)-3-carboxamide

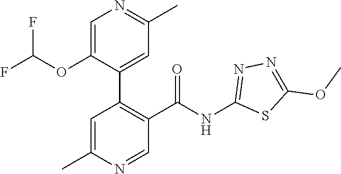 [SMILES: COc1nnc(NC(=O)c2cnc(C)cc2-c2cc(C)ncc2OC(F)F)s1]

To a stirred solution of 5′-(difluoromethoxy)-2′,6-dimethyl-(4,4′-bipyridine)-3-carboxylic acid (60.0 mg, 0.20 mmol) and 5-methoxy-1,3,4-thiadiazol-2-amine (26.0 mg, 0.20 mmol) in Acetonitrile (1 mL) was added 1-methyl-1H-imidazole (84.0 mg, 1.02 mmol) at 25° C. Then a solution of TCFH (56.0 mg, 0.20 mmol) in acetonitrile (0.5 mL) was added to the above mixture at 25° C. The mixture was stirred at 25° C. for 1 hr. The resulting mixture was applied to a 20 g C18 column and purified by Combi Flash (Biotage Isolera Prime), eluted with 5˜38% acetonitrile in water within 30 min to afford 5′-(difluoromethoxy)-N-(5-methoxy-1,3,4-thiadiazol-2-yl)-2′,6-dimethyl-(4,4′-bipyridine)-3-carboxamide (25.0 mg, 29%) as a white solid. MS (ESI) calc'd for (C₁₇H₁₅F₂N₅O₃S) (M+1)⁺, 408.1. found 408.1. ¹H NMR (400 MHz, DMSO-d₆) δ 13.01 (s, 1H), 8.91 (s, 1H), 8.32 (s, 1H), 7.42-7.32 (m, 2H), 7.22-6.85 (m, 1H), 4.07 (s, 3H), 2.60 (s, 3H), 2.53 (s, 3H).

### Example 53

### 4-(5-chloro-2-ethoxyphenyl)-N-(5-methoxy-1,3,4-thiadiazol-2-yl)-6-methylnicotinamide

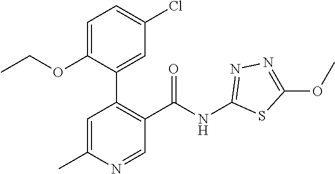 [SMILES: CCOc1ccc(Cl)cc1-c1cc(C)ncc1C(=O)Nc1nnc(OC)s1]

### Step 1 methyl 4-(5-chloro-2-ethoxyphenyl)-6-methylnicotinate

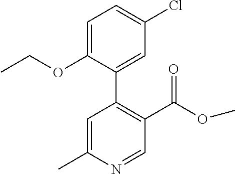 [SMILES: CCOc1ccc(Cl)cc1-c1cc(C)ncc1C(=O)OC]

To a solution of methyl 4-chloro-6-methylnicotinate (500.0 mg, 2.56 mmol) in 1,4-Dioxane (8 mL) were added (5-chloro-2-ethoxyphenyl) boronic acid (513.0 mg, 2.56 mmol), Pd(dppf)Cl₂ (209.0 mg, 0.25 mmol), K₂CO₃ (707.0 mg, 5.12 mmol) and Water (2 mL) at 18 ºC. The resulting solution was stirred at 80° C. for 2 hr. The reaction mixture was quenched by the addition of water and extracted with ethyl acetate. The combined organic layers were washed with brine, dried over anhydrous sodium sulfate, filtered, and concentrated under vacuum. The residue was dissolved in DCM (6 mL) and purified by Combi Flash (Biotage Isolera Prime) which applied to a 80 g silica gel column that was eluted with 0˜45% ethyl acetate in petroleum ether within 40 min to afford methyl 4-(5-chloro-2-ethoxyphenyl)-6-methylnicotinate (680.0 mg, 70%) as a white solid. MS (ESI) calc'd for (C₁₆H₁₆ClNO₃) (M+1)⁺, 306.0. found 306.0.

### Step 2 4-(5-chloro-2-ethoxyphenyl)-6-methylnicotinic Acid

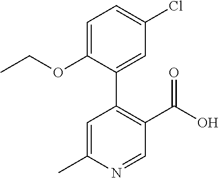 [SMILES: CCOc1ccc(Cl)cc1-c1cc(C)ncc1C(=O)O]

To a solution of methyl 4-(5-chloro-2-ethoxyphenyl)-6-methylnicotinate (200.0 mg, 0.52 mmol) in Tetrahydrofuran (1.5 mL) were added LiOH (37.6 mg, 1.57 mmol) and Water (0.5 mL) at 20° C. The resulting solution was stirred at 50° C. for 16 hr. The reaction mixture was diluted with water. The aqueous layer was acidified with citric acid to pH ˜5 and extracted with ethyl acetate. The combined organic layers were washed with brine, dried over anhydrous sodium sulfate, filtered, and concentrated under vacuum to afford 4-(5-chloro-2-ethoxyphenyl)-6-methylnicotinic acid (160.0 mg, crude) as a yellow solid. MS (ESI) calc'd for (C₁₅H₁₄ClNO₃) (M+1)⁺, 292.0. found 292.0.

### Step 3 4-(5-chloro-2-ethoxyphenyl)-N-(5-methoxy-1,3,4-thiadiazol-2-yl)-6-methylnicotinamide

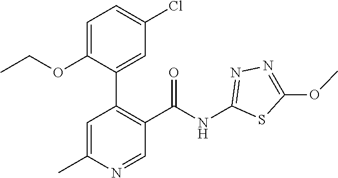 [SMILES: CCOc1ccc(Cl)cc1-c1cc(C)ncc1C(=O)Nc1nnc(OC)s1]

To a mixture of 4-(5-chloro-2-ethoxyphenyl)-6-methylnicotinic acid (160.0 mg, 0.43 mmol) in Acetonitrile (1 mL) were added 5-methoxy-1,3,4-thiadiazol-2-amine (69.1 mg, 0.52 mmol) and 1-methyl-1H-imidazole (108.0 mg, 1.31 mmol). A solution of TCFH (184.0 mg, 0.65 mmol) in Acetonitrile (1 mL) was added thereto dropwise under nitrogen. The resulting solution was stirred at 20° C. for 2 hr. The organic solvent was removed under vacuum. The residue was dissolved in DMF (3 mL) which was applied to a 40 g C18 column and purified by Combi Flash (Biotage Isolera Prime), eluted with 5˜46% acetonitrile in water within 40 min to afford 4-(5-chloro-2-ethoxyphenyl)-N-(5-methoxy-1,3,4-thiadiazol-2-yl)-6-methylnicotinamide (42.0 mg, 23%) as a white solid. MS (ESI) calc'd for (C₁₈H₁₇ClN₄O₃S) (M+1)⁺, 405.0. found 405.0. ¹H NMR (400 MHz, DMSO-d₆) δ 12.74 (s, 1H), 8.73 (s, 1H), 7.40-7.33 (m, 3H), 6.97-6.99 (d, J=8.8 Hz, 1H), 4.07 (s, 3H), 3.78 (d, J=6.8 Hz, 2H), 2.58 (s, 3H), 1.06-1.09 (t, J=6.8 Hz, 3H).

### Example 54

### N-(5-methoxy-1,3,4-thiadiazol-2-yl)-4-(2-methoxyphenyl)-6-methylnicotinamide

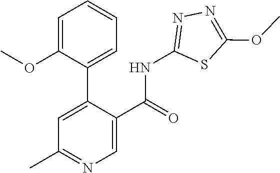

To a stirred solution of Intermediate C (310.0 mg, 1.27 mmol) and 5-methoxy-1,3,4-thiadiazol-2-amine (167.0 mg, 1.27 mmol) in acetonitrile (2 mL) was added 1-methylimidazole (522.3 mg, 6.37 mmol). Then TCFH (358.0 mg, 1.27 mmol) in acetonitrile (1 mL) was added to the above mixture at 23° C. The resulting solution was stirred at 23° C. for 2 hr under nitrogen. The resulting residue was dissolved in acetonitrile (2 mL) which was applied to a 40.0 g C18 column and purified by Combi Flash (Biotage Isolera Prime), eluted with 5˜38% acetonitrile in water within 30 min to afford N-(5-methoxy-1,3,4-thiadiazol-2-yl)-4-(2-methoxyphenyl)-6-methylnicotinamide (219.0 mg, 47% yield) as a white solid. MS (ESI) calc'd for (C₁₇H₁₆N₄O₃S) (M+1)⁺, 357.1. found 357.1. ¹H NMR (400 MHz, DMSO-d₆) δ 12.71 (s, 1H), 8.67 (s, 1H), 7.45-7.23 (m, 3H), 7.12-7.02 (m, 1H), 7.02-6.95 (m, 1H), 4.07 (s, 3H), 3.64 (s, 3H), 2.57 (s, 3H).

### Example 55

### 4-(2-chloro-6-(difluoromethoxy)phenyl)-N-(5-methoxy-1,3,4-thiadiazol-2-yl)-6-methylnicotinamide

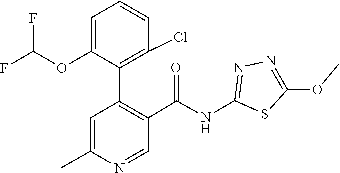 [SMILES: COc1nnc(NC(=O)c2cnc(C)cc2-c2c(Cl)cccc2OC(F)F)s1]

### Step-1: 2-Bromo-1-chloro-3-(difluoromethoxy)benzene

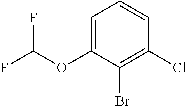 [SMILES: FC(F)Oc1cccc(Cl)c1Br]

To a stirred solution of 2-bromo-3-chlorophenol (5.0 g, 24.10 mmol) in N,N-Dimethylformamide (30 mL) were added K₂CO₃ (10.0 g, 72.30 mmol) in Water (3 mL) at 23° C. To the above solution was added Sodium chlorodifluoroacetate (7.4 g, 48.20 mmol) at 23° C. under nitrogen. The resulting solution was stirred at 100° C. for 16 hr. The reaction mixture was extracted with ethyl acetate. The combined organic layers were washed with brine, dried over anhydrous sodium sulfate, filtered, and concentrated under vacuum. The resulting residue was dissolved in DCM (4 mL) and purified by Combi Flash (Biotage Isolera Prime) which applied to a 120 g silica gel column that was eluted with 0˜20% ethyl acetate in petroleum ether within 25 min to afford 2-bromo-1-chloro-3-(difluoromethoxy)benzene (4.9 g, 75%) as a colorless oil. MS (ESI) calc'd for (C₇H₄BrClF₂O) (M+1)⁺, 256.9. found, 257.0.

### Step-2: 2-(2-chloro-6-(difluoromethoxy)phenyl)-4,4,5,5-tetramethyl-1,3,2-dioxaborolane

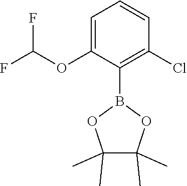 [SMILES: CC1(C)OB(c2c(Cl)cccc2OC(F)F)OC1(C)C]

To a stirred solution of 2-bromo-1-chloro-3-(difluoromethoxy) benzene (4.9 g, 19.03 mmol) in 1,4-Dioxane (20 mL) were added Bis(pinacolato)diboron (9.7 g, 38.10 mmol), AcOK (3.7 g, 38.10 mmol) and Pd(dppf)Cl₂ (1.6 g, 1.90 mmol) at 23° C. The resulting solution was stirred at 100° C. for 3 hr under nitrogen. The organic solvent was removed under vacuum. The resulting residue was purified by Combi Flash (Biotage Isolera Prime) which applied to 120 g silica gel column and eluted with 0˜10% ethyl acetate in petroleum ether within 20 min to afford 2-(2-chloro-6-(difluoromethoxy)phenyl)-4,4,5,5-tetramethyl-1,3,2-dioxaborolane (1.6 g, 22%) as a colorless oil. MS (ESI) calc'd for (C₁₃H₁₆BClF₂O₃) (M+1)⁺, 305.1. found, 305.0.

### Step-3: methyl 4-(2-chloro-6-(difluoromethoxy)phenyl)-6-methylnicotinate

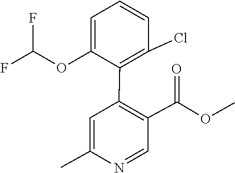 [SMILES: COC(=O)c1cnc(C)cc1-c1c(Cl)cccc1OC(F)F]

To a stirred solution of 2-(2-chloro-6-(difluoromethoxy)phenyl)-4,4,5,5-tetramethyl-1,3,2-dioxaborolane (1.6 g, 5.25 mmol) in 1,4-Dioxane (6 mL) were added methyl 4-iodo-6-methylnicotinate (728.1 mg, 2.63 mmol, K₂CO₃ (1.4 g, 10.51 mmol) and Water (1.2 mL). To the above solution was added Pd(dppf)Cl₂ (429.0 mg, 0.53 mmol) at 23° C. under nitrogen. The resulting solution was stirred at 100° C. for 16 hr under nitrogen. The reaction mixture was diluted with water, extracted with ethyl acetate. The combined organic layers were washed with brine, dried over anhydrous sodium sulfate, filtered, and concentrated under vacuum. The resulting residue was dissolved in DMF (3 mL) which was applied to a 40 g C18 column and purified by Combi Flash (Biotage Isolera Prime), eluted with 5˜50% acetonitrile in water within 30 min to afford methyl 4-(2-chloro-6-(difluoromethoxy)phenyl)-6-methylnicotinate (140.0 mg, 8%) as a white solid. MS (ESI) calc'd for (C₁₅H₁₂ClF₂NO₃) (M+1)⁺, 328.0. found 328.2.

### Step-4: 4-(2-chloro-6-(difluoromethoxy)phenyl)-6-methylnicotinic Acid

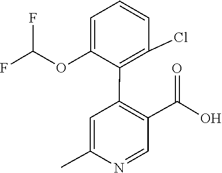 [SMILES: Cc1cc(-c2c(Cl)cccc2OC(F)F)c(C(=O)O)cn1]

To a stirred solution of methyl 4-(2-chloro-6-(difluoromethoxy)phenyl)-6-methylnicotinate (140.0 mg, 0.43 mmol) in Tetrahydrofuran (1 mL) and Water (1 mL) was added LiOH (20.0 mg, 0.85 mmol) at 23° C. The resulting solution was stirred at 23° C. for 3 hr. The mixture was diluted with water. The aqueous layer was acidified with Citric acid to pH ˜3 and extracted with ethyl acetate. The combined organic layers were washed with brine, dried over anhydrous sodium sulfate, filtered, and concentrated under vacuum. The resulting residue was dissolved in DMF (2 mL) which was applied to a 40 g C18 column and purified by Combi Flash (Biotage Isolera Prime), eluted with 5˜50% acetonitrile in water within 25 min to afford 4-(2-chloro-6-(difluoromethoxy)phenyl)-6-methylnicotinic acid (60.0 mg, 44%) as a white solid. MS (ESI) calc'd for (C₁₄H₁₀ClF₂NO₃) (M+1)⁺, 314.0. found, 314.0.

### Step-5: 4-(2-chloro-6-(difluoromethoxy)phenyl)-N-(5-methoxy-1,3,4-thiadiazol-2-yl)-6-methylnicotinamide

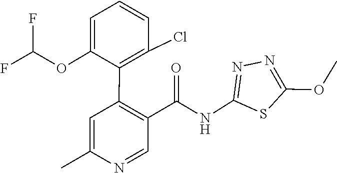 [SMILES: COc1nnc(NC(=O)c2cnc(C)cc2-c2c(Cl)cccc2OC(F)F)s1]

To a stirred solution of 4-(2-chloro-6-(difluoromethoxy)phenyl)-6-methylnicotinic acid (50.0 mg, 0.159 mmol) in Acetonitrile (1 mL) were added 5-methoxy-1,3,4-thiadiazol-2-amine (21.0 mg, 0.16 mmol) and 1-methylimidazole (65.0 mg, 0.79 mmol). To the above solution was added a solution of TCFH (45.0 mg, 0.16 mmol) in Acetonitrile (0.5 mL). The mixture was then stirred at 23° C. for 2 hr. The resulting mixture was dissolved in DMF (1 mL) which was applied to a 20 g C18 column and purified by Combi Flash (Biotage Isolera Prime), eluted with 5˜50% acetonitrile in water within 30 min to afford 4-(2-chloro-6-(difluoromethoxy)phenyl)-N-(5-methoxy-1,3,4-thiadiazol-2-yl)-6-methylnicotinamide (40.6 mg, 59%) as a white solid. MS (ESI) calc'd for (C₁₇H₁₃ClF₂N₄O₃S) (M+1)⁺, 427.0. found, 427.2. ¹H NMR (400 MHz, DMSO-d₆) δ 13.01 (s, 1H), 8.99 (s, 1H), 7.57-7.41 (m, 2H), 7.30-7.22 (m, 2H), 7.22-6.93 (m, 1H), 4.06 (s, 3H), 2.60 (s, 3H).

### Example 56

### N-(5-methoxy-1,3,4-thiadiazol-2-yl)-2′,5′,6-trimethyl-(4,4′-bipyridine)-3-carboxamide

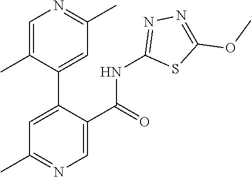

### Step 1 methyl 2′-chloro-5′,6-dimethyl-(4,4′-bipyridine)-3-carboxylate

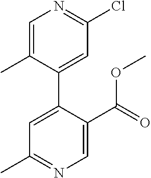

To a stirred solution of 4-bromo-2-chloro-5-methylpyridine (1.0 g, 4.84 mmol) in 1,4-Dioxane (8 mL) were sequentially added methyl 6-methyl-4-(4,4,5,5-tetramethyl-1,3,2-dioxaborolan-2-yl)nicotinate (1.3 g, 4.84 mmol), water (1.6 mL) and K₂CO₃ (2.0 g, 14.53 mmol) at 23° C. To the above solution was added PdCl2(dppf) (354.0 mg, 0.48 mmol) at 23° C. The resulting solution was stirred at 80° C. for 2 hr. The suspension was filtered. The filtrate was collected and concentrated under vacuum. The resulting residue was dissolved in DMF (3 mL) which was applied to a 40 g C18 column and purified by Combi Flash (Biotage Isolera Prime), eluted with 5˜60% acetonitrile in water within 30 min to afford methyl 2′-chloro-5′,6-dimethyl-(4,4′-bipyridine)-3-carboxylate (588.0 mg, 37%) as a brown oil. MS (ESI) calculated for (C₁₄H₁₃ClN₂O₂) (M+1)⁺, 277.1. found, 277.1.

### Step 2 methyl 2′,5′,6-trimethyl-(4,4′-bipyridine)-3-carboxylate

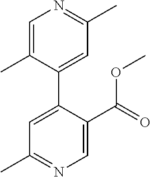 [SMILES: COC(=O)c1cnc(C)cc1-c1cc(C)ncc1C]

To a stirred solution of methyl 2′-chloro-5′,6-dimethyl-(4,4′-bipyridine)-3-carboxylate (150.0 mg, 0.54 mmol) in 1,4-Dioxane (1 mL) were sequentially added Trimethylboroxine (136.0 mg, 1.08 mmol), K₂CO₃ (225.0 mg, 1.63 mmol) and water (0.2 mL) at 23° C. To the above solution was added PdCl2(dppf) (39.7 mg, 0.05 mmol) at 25° C. under nitrogen. The resulting mixture was then stirred at 120° C. for 1 hr under nitrogen. The volatiles were removed under vacuum and purified directly. The resulting residue was dissolved in DMF (3 mL) which was applied to an 80 g C18 column and purified by Combi Flash (Biotage Isolera Prime), eluted with 5˜50% acetonitrile in water within 30 min to afford methyl 2′,5′,6-trimethyl-(4,4′-bipyridine)-3-carboxylate (60.0 mg, 41%) as a brown oil. MS (ESI) calculated for (C₁₅H₁₆N₂O₂) (M+1)⁺, 257.1. found, 257.1.

### Step 3 2′,5′,6-trimethyl-(4,4′-bipyridine)-3-carboxylic Acid

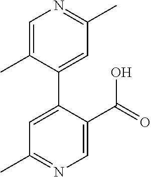 [SMILES: Cc1cc(-c2cc(C)ncc2C(=O)O)c(C)cn1]

To a stirred solution of methyl 2′,5′,6-trimethyl-(4,4′-bipyridine)-3-carboxylate (160.0 mg, 0.63 mmol) in Methanol (2 mL) was added sodium hydroxide (50.4 mg, 1.26 mmol) in water (1 mL) at 20° C. The resulting solution was stirred at room temperature for 4 hr. The organic solvent was removed under vacuum. The aqueous layer was acidified with citric acid to pH 5˜6 and extracted with ethyl acetate. The combined organic layers were washed with brine, dried over anhydrous sodium sulfate, filtered, and concentrated under vacuum. The resulting residue was dissolved in DMF (2 mL) which was applied to an 40 g C18 column and purified by Combi Flash (Biotage Isolera Prime), eluted with 5˜30% acetonitrile in water within 30 min to afford 2′,5′,6-trimethyl-(4,4′-bipyridine)-3-carboxylic acid (36.0 mg, 24%) as a yellow solid. MS (ESI) calculated for (C₁₄H₁₄N₂O₂) (M+1)⁺, 243.1. found, 243.1.

### Step 4 N-(5-methoxy-1,3,4-thiadiazol-2-yl)-2′,5′,6-trimethyl-(4,4′-bipyridine)-3-carboxamide

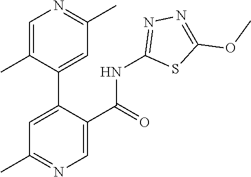

To a stirred solution of 2′,5′,6-trimethyl-(4,4′-bipyridine)-3-carboxylic acid (36.0 mg, 0.15 mmol) and 5-methoxy-1,3,4-thiadiazol-2-amine (19.5 mg, 0.15 mmol) in acetonitrile (1 mL) was added 1-methyl-1H-imidazole (61.0 mg, 0.74 mmol) at 25° C. To the above solution was added a solution of TCFH (41.7 mg, 0.15 mmol) in acetonitrile (0.5 mL) at 25° C. The resulting solution was stirred at 25° C. for 2 hr under nitrogen. The mixture was dissolved in DMF (3 mL) and was purified by prep-HPLC with the following condition: (Column: XBridge Prep OBD C18 Column, 30*150 mm, 5 um; Mobile Phase A: Water (10 mmol/L NH₄HCO₃), Mobile Phase B: ACN; Flow rate: 60 mL/min; Gradient: 10% B to 30% B in 8 min, 30% B to 95% B in 8.2 min, 95% B to 95% B in 9.5 min, 95% B to 5% B in 11 min, 5% B; Wave Length: 254 nm; RT1 (min): 7; Injection Volume: 1 mL; Number Of Runs: 4) to afford N-(5-methoxy-1,3,4-thiadiazol-2-yl)-2′,5′,6-trimethyl-(4,4′-bipyridine)-3-carboxamide (26.1 mg, 49%) as a white solid. MS (ESI) calculated for (C₁₇H₁₇N₅O₂S) (M+1)⁺, 356.1. found, 356.1. ¹H NMR (400 MHz, DMSO-d₆) δ 12.99 (br, 1H), 8.90 (s, 1H), 8.31 (s, 1H), 7.23 (s, 1H), 6.99 (s, 1H), 4.04 (s, 3H), 2.58 (s, 3H), 2.42 (s, 3H), 1.97 (s, 3H).

### Example 57

### 5′-methoxy-N-(5-methoxy-1,3,4-thiadiazol-2-yl)-6-methyl-2′-(trifluoromethyl)-(4,4′-bipyridine)-3-carboxamide

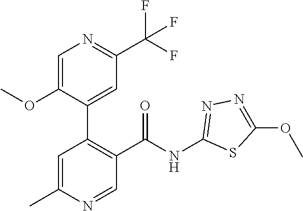

### Step-1: 5-methoxy-2-(trifluoromethyl)pyridine

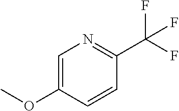

To a stirred solution of 6-(trifluoromethyl)pyridin-3-ol (4.0 g, 24.52 mmol) in N,N-Dimethylformamide (DMF) (40 mL) were added K₂CO₃ (10.2 g, 73.6 mmol) and MeI (1.8 mL, 29.4 mmol) at 23° C. The resulting solution was stirred at 23° C. for 2 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (3 mL) and purified by Combi Flash (Biotage Isolera Prime) which applied to a 40 g silica gel column that was eluted with 0˜10% ethyl acetate in petroleum ether within 20 min to afford 5-methoxy-2-(trifluoromethyl)pyridine (3.5 g, 80%) as a colorless oil. MS (ESI) calculated for (C₇H₆F₃NO) (M+1)⁺, 178.0. found, 178.1.

### Step-2: 4-iodo-5-methoxy-2-(trifluoromethyl)pyridine

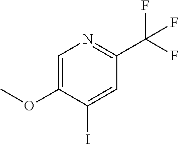

To a degassed solution of 5-methoxy-2-(trifluoromethyl)pyridine (1.0 g, 5.65 mmol) in dry tetrahydrofuran (THF) (5 mL) was added n-butyllithium (4.5 mL, 11.29 mmol, 2.5 M in hexane) dropwise at −60° C. and stirred at −60° C. for 1 hr under nitrogen atmosphere. Then a solution of I₂ (2.8 g, 11.29 mmol) in THF was added to the above mixture at −60° C. The resulting solution was stirred from −60° C. to 23° C. for 2 hr. The reaction mixture was quenched by the addition of water, extracted with ethyl acetate. The combined organic layers were washed with brine, dried over anhydrous sodium sulfate, filtered, and concentrated under vacuum. The resulting residue was dissolved in DMF (8 mL) which was applied to a 80 g C18 column and purified by Combi Flash (Biotage Isolera Prime), eluted with 5˜90% acetonitrile in water within 35 min to afford 4-iodo-5-methoxy-2-(trifluoromethyl)pyridine (135.0 mg, 8% yield) as a yellow solid. MS (ESI) calculated for (C₇H₅F₃INO) (M+1)⁺, 304. found, 353.1.

### Step-3: methyl 5′-methoxy-6-methyl-2′-(trifluoromethyl)-(4,4′-bipyridine)-3-carboxylate

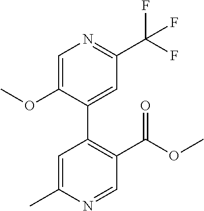

To a stirred solution of 4-iodo-5-methoxy-2-(trifluoromethyl)pyridine (300.0 mg, 0.99 mmol) in 1,4-Dioxane (1.5 mL) were sequentially added methyl 6-methyl-4-(4,4,5,5-tetramethyl-1,3,2-dioxaborolan-2-yl)nicotinate (412.0 mg, 1.49 mmol), water (0.3 mL) and K₂CO₃ (410.0 mg, 2.97 mmol) at 23° C. To the above solution was added PdCl₂(dppf) (72.4 mg, 0.10 mmol) at 23° C. The resulting solution was stirred at 80° C. for 2 hr under nitrogen. The resulting mixture was dissolved in DMF (4 mL) and was filtered. The filtrate was applied to a 40 g C18 column and purified by Combi Flash (Biotage Isolera Prime), eluted with 5˜70% acetonitrile in water within 30 min to afford methyl 5′-methoxy-6-methyl-2′-(trifluoromethyl)-(4,4′-bipyridine)-3-carboxylate (140.0 mg, 43% yield) as a yellow oil. MS (ESI) calculated for (C₁₅H₁₃F₃N₂O₃) (M+1)⁺, 327.0. found, 327.1.

### Step-4: 5′-methoxy-6-methyl-2′-(trifluoromethyl)-(4,4′-bipyridine)-3-carboxylic Acid

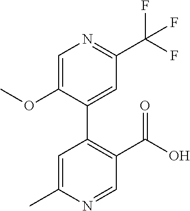 [SMILES: COc1cnc(C(F)(F)F)cc1-c1cc(C)ncc1C(=O)O]

To a stirred solution of methyl 5′-methoxy-6-methyl-2′-(trifluoromethyl)-(4,4′-bipyridine)-3-carboxylate (140.0 mg, 0.43 mmol) in methanol (1 mL) were added NaOH (68.6 mg, 1.72 mmol) and water (1.0 mL) at 23° C. The resulting solution was stirred at 50° C. for 1 hr before diluted with water. The organic solvent was removed under vacuum. The aqueous layer was acidified with citric acid to pH ˜6 and extracted with ethyl acetate. The combined organic layers were washed with brine, dried over anhydrous sodium sulfate, filtered, and concentrated under vacuum. The resulting residue was dissolved in DMF (1 mL) which was applied to a 25 g C18 column and purified by Combi Flash (Biotage Isolera Prime), eluted with 5˜60% acetonitrile in water within 30 min to afford 5′-methoxy-6-methyl-2′-(trifluoromethyl)-(4,4′-bipyridine)-3-carboxylic acid (80.0 mg, 59%) as a white solid. MS (ESI) calculated for (C₁₄H₁₁F₃N₂O₃) (M+1)⁺, 313.0. found, 313.1.

### Step-5: 5′-methoxy-N-(5-methoxy-1,3,4-thiadiazol-2-yl)-6-methyl-2′-(trifluoromethyl)-(4,4′-bipyridine)-3-carboxamide

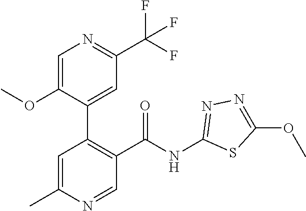

To a stirred solution of 5′-methoxy-6-methyl-2′-(trifluoromethyl)-(4,4′-bipyridine)-3-carboxylic acid (47.0 mg, 0.15 mmol) in acetonitrile (1 mL) under argon atmosphere were added 5-methoxy-1,3,4-thiadiazol-2-amine (21.7 mg, 0.17 mmol) and 1-Methylimidazole (61.5 mg, 0.75 mmol) at 23° C. To the above solution was added a solution of TCFH (0.15 mmol) in acetonitrile (0.5 mL) at 23° C. under nitrogen. The resulting mixture was then stirred at 23° C. for 1 hr. The solvents were removed under vacuum. The residue was dissolved in DMF (2 mL) and was purified by prep-HPLC with the following condition: (Column: XBridge Prep OBD C18 Column, 30*150 mm, 5 um; Mobile Phase A: Water (10 mmol/L NH₄HCO₃), Mobile Phase B: ACN; Flow rate: 60 mL/min; Gradient: 25% B to 35% B in 8 min, 35% B to 40% B in 15 min, 40% B; Wave Length: 254 nm; RT1 (min): 7.7) to afford 5′-methoxy-N-(5-methoxy-1,3,4-thiadiazol-2-yl)-6-methyl-2′-(trifluoromethyl)-(4,4′-bipyridine)-3-carboxamide (14.3 mg, 22%) as a white solid. MS (ESI) calculated for (C₁₇H₁₄F₃N₅O₃S) (M+1)⁺, 426. found, 426.10. ¹H NMR (400 MHz, DMSO-d₆) δ 12.87 (s, 1H), 8.86 (s, 1H), 8.54 (s, 1H), 7.88 (s, 1H), 7.43 (s, 1H), 4.05 (s, 3H), 3.74 (s, 3H), 2.59 (s, 3H).

### Example 58 and 59

### 2′-chloro-N-(5-(((1S,3R)-3-hydroxycyclopentyl)oxy)-1,3,4-thiadiazol-2-yl)-5′-methoxy-6-methyl-(4,4′-bipyridine)-3-carboxamide and 2′-chloro-N-(5-(((1R,3S)-3-hydroxycyclopentyl)oxy)-1,3,4-thiadiazol-2-yl)-5′-methoxy-6-methyl-(4,4′-bipyridine)-3-carboxamide

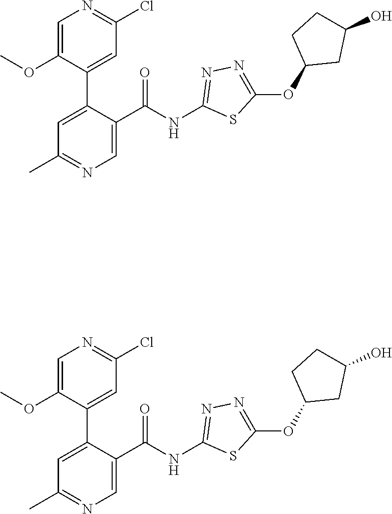 [SMILES: COc1cnc(Cl)cc1-c1cc(C)ncc1C(=O)Nc1nnc(O[C@H]2CC[C@@H](O)C2)s1 ; COc1cnc(Cl)cc1-c1cc(C)ncc1C(=O)Nc1nnc(O[C@@H]2CC[C@H](O)C2)s1]

The mixture of compounds 2′-chloro-N-(5-(((1s,3r)-3-hydroxycyclopentyl)oxy)-1,3,4-thiadiazol-2-yl)-5′-methoxy-6-methyl-[4,4′-bipyridine]-3-carboxamide (42.6 mg) was separated by prep-chiral HPLC with the following condition: (Column: CHIRALPAK IE, 2*25 cm, 5 μm; Mobile Phase A: Hex:DCM=3: 1 (0.1% FA)—HPLC, Mobile Phase B: EtOH—HPLC; Flow rate: 15 mL/min; Gradient: 50% B to 50% B in 34 min; Wave Length: 220/254 nm; RT1 (min): 18.26; RT2 (min): 26.54; Sample Solvent: MeOH:DCM=1:1; Injection Volume: 1.5 mL; Number Of Runs: 2) to afford 2′-chloro-N-(5-(((1S,3R)-3-hydroxycyclopentyl)oxy)-1,3,4-thiadiazol-2-yl)-5′-methoxy-6-methyl-(4,4′-bipyridine)-3-carboxamide (16.2 mg, 38% yield) as a white solid with the first peak on chiral HPLC and 2′-chloro-N-(5-(((1R,3S)-3-hydroxycyclopentyl)oxy)-1,3,4-thiadiazol-2-yl)-5′-methoxy-6-methyl-(4,4′-bipyridine)-3-carboxamide (16.6 mg, 39% yield) as a white solid with the second peak on chiral HPLC. The absolute stereochemistry was not determined and was arbitrarily assigned.

2′-chloro-N-(5-(((1S,3R)-3-hydroxycyclopentyl)oxy)-1,3,4-thiadiazol-2-yl)-5′-methoxy-6-methyl-(4,4′-bipyridine)-3-carboxamide: MS (ESI) calculated for (C₂₀H₂₀ClN₅O₄S) (M+1)⁺, 462.1. found, 462.1, ¹H NMR (400 MHz, DMSO-d₆) δ 12.87 (s, 1H), 8.82 (s, 1H), 8.16 (s, 1H), 7.51 (s, 1H), 7.39 (s, 1H), 7.14 (s, 1H), 5.39-5.26 (m, 1H), 4.64-4.50 (m, 1H), 4.27-4.13 (m, 1H), 3.63 (s, 3H), 2.58 (s, 3H), 2.22-2.15 (m, 1H), 2.01-1.99 (m, 2H), 1.93-1.91 (m, 1H), 1.89-1.88 (m, 1H).

2′-chloro-N-(5-(((1R,3S)-3-hydroxycyclopentyl)oxy)-1,3,4-thiadiazol-2-yl)-5′-methoxy-6-methyl-(4,4′-bipyridine)-3-carboxamide: MS (ESI) calculated for (C₂₀H₂₀ClN₅O₄S) (M+1)⁺, 462.1. found, 462.1. ¹H NMR (400 MHz, DMSO-d₆) δ 12.87 (s, 1H), 8.82 (s, 1H), 8.16 (s, 1H), 7.51 (s, 1H), 7.39 (s, 1H), 7.14 (s, 1H), 5.39-5.26 (m, 1H), 4.64-4.50 (m, 1H), 4.27-4.13 (m, 1H), 3.63 (s, 3H), 2.58 (s, 3H), 2.22-2.15 (m, 1H), 2.01-1.99 (m, 2H), 1.93-1.91 (m, 1H), 1.89-1.88 (m, 1H).

### Example 60

### 2′-chloro-3′-fluoro-5′-methoxy-6-methyl-N-(5-(oxetan-3-yloxy)-1,3,4-thiadiazol-2-yl)-(4,4′-bipyridine)-3-carboxamide

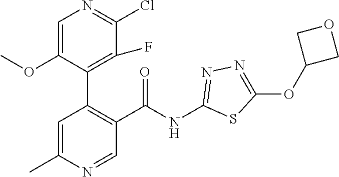 [SMILES: COc1cnc(Cl)c(F)c1-c1cc(C)ncc1C(=O)Nc1nnc(OC2COC2)s1]

### Step 1 2-chloro-3-fluoro-5-methoxypyridine

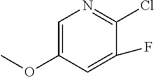

To a solution of 6-chloro-5-fluoropyridin-3-ol (3.0 g, 20.33 mmol) in acetone (15 mL) was added MeI (2.5 mL, 40.7 mmol) and K₂CO₃ (5.6 g, 40.7 mmol) at 20° C. under nitrogen atmosphere. The resulting solution was stirred at 20° C. for 18 hr under nitrogen. The reaction mixture was extracted with ethyl acetate. The combined organic layers were washed with brine, dried over anhydrous sodium sulfate, filtered, and concentrated under vacuum. The resulting residue was dissolved in dichloromethane (5 mL) and purified by Combi Flash

(Biotage Isolera Prime) which applied to a 120.0 g silica gel column that was eluted with 0˜30% ethyl acetate in petroleum ether within 25 min to afford 2-chloro-3-fluoro-5-methoxypyridine (2.1 g, 63% yield) as a colorless oil. MS (ESI) calc'd for (C₆H₅ClFNO) (M+1)⁺, 162.0. found 162.0.

### Step 2 2-chloro-3-fluoro-4-iodo-5-methoxypyridine

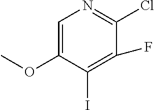

To a degassed solution of 2-chloro-3-fluoro-5-methoxypyridine (2.1 g, 13.00 mmol) in dry Tetrahydrofuran (5 mL) was added n-butyllithium (5.8 mL, 14.30 mmol, 2.5 M in hexane) dropwise at −60° C. and stirred at −60° C. for 1 hr under nitrogen atmosphere. Then iodine (3.6 g, 14.30 mmol) was added to the above mixture at −60° C. The resulting solution was stirred at 20° C. for 2 hr under nitrogen. The reaction mixture was quenched by the addition of saturated sodium thiosulfate aqueous solution and extracted with ethyl acetate. The combined organic layers were washed with brine, dried over anhydrous sodium sulfate, filtered, and concentrated under vacuum. The resulting residue was dissolved in dichloromethane (2 mL) and purified by Combi Flash (Biotage Isolera Prime) which applied to a 40 g silica gel column that was eluted with 0˜15% ethyl acetate in petroleum ether within 30 min to afford 2-chloro-3-fluoro-4-iodo-5-methoxypyridine (3.5 g, 89% yield) as a white solid. MS (ESI) calc'd for (C₆H₄ClFINO) (M+1)⁺, 287.9. found, 288.0.

### Step 3 methyl 2′-chloro-3′-fluoro-5′-methoxy-6-methyl-(4,4′-bipyridine)-3-carboxylate

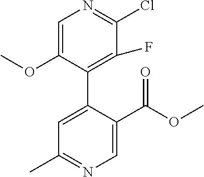

To a degassed solution of methyl 6-methyl-4-(4,4,5,5-tetramethyl-1,3,2-dioxaborolan-2-yl)nicotinate (3.0 g, 10.83 mmol) and 2-chloro-3-fluoro-4-iodo-5-methoxypyridine (2.1 g, 7.31 mmol) in 1,4-dioxane (20 mL) were sequentially added water (4 mL), potassium carbonate (3.0 g, 21.71 mmol) and (1,1′-Bis(diphenylphosphino)ferrocene)dichloropalladium(II) (1.6 g, 2.19 mmol) at 25° C. The resulting solution was stirred at 80° C. for 2 hr under nitrogen. The suspension was filtered. The filtrate was collected and concentrated under vacuum. The resulting residue was dissolved in dichloromethane (10 mL) and purified by Combi Flash (Biotage Isolera Prime) which applied to a 120 g silica gel column that was eluted with 0˜50% ethyl acetate in petroleum ether within 30 min to afford methyl 4-(2-(difluoromethoxy)phenyl)-6-methylnicotinate (2.2 g, 87% yield) as a white solid. MS (ESI) calc'd for (C₁₄H₁₂ClFN₂O₃) (M+1)⁺, 311.1. found 311.1.

### Step 4 2′-chloro-3′-fluoro-5′-methoxy-6-methyl-(4,4′-bipyridine)-3-carboxylic Acid

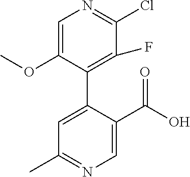 [SMILES: COc1cnc(Cl)c(F)c1-c1cc(C)ncc1C(=O)O]

To a stirred solution of methyl 2′-chloro-3′-fluoro-5′-methoxy-6-methyl-(4,4′-bipyridine)-3-carboxylate (800.0 mg, 2.57 mmol) in methanol (5 mL) were added water (5 mL) and sodium hydroxide (412.0 mg, 10.30 mmol) at 23° C. The resulting solution was stirred at 23° C. for 1 hr. The organic solvent was removed under vacuum. The aqueous layer was acidified with sat. citric acid solution to pH ˜6. The resulting mixture was dissolved in acetonitrile (3 mL) which was applied to a 40.0 g C18 column and purified by Combi Flash (Biotage Isolera Prime), eluted with 0˜10% acetonitrile in water within 30 min to afford 2′-chloro-3′-fluoro-5′-methoxy-6-methyl-(4,4′-bipyridine)-3-carboxylic acid (260.0 mg, 32% yield) as a yellow oil. MS (ESI) calc'd for (C₁₃H₁₀ClFN₂O₃) (M+1)⁺, 297.0. found 297.0.

### Step 5 2′-chloro-3′-fluoro-5′-methoxy-6-methyl-N-(5-(oxetan-3-yloxy)-1,3,4-thiadiazol-2-yl)-(4,4′-bipyridine)-3-carboxamide

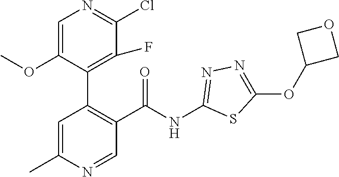 [SMILES: COc1cnc(Cl)c(F)c1-c1cc(C)ncc1C(=O)Nc1nnc(OC2COC2)s1]

To a stirred solution of 2′-chloro-3′-fluoro-5′-methoxy-6-methyl-(4,4′-bipyridine)-3-carboxylic acid (100 mg, 0.34 mmol) and 5-(oxetan-3-yloxy)-1,3,4-thiadiazol-2-amine (58.4 mg, 0.34 mmol, Example 13, Step 1) in acetonitrile (1 mL) was added 1-methylimidazole (110.7 mg, 1.35 mmol). Then TCFH (95.0 mg, 0.34 mmol) in acetonitrile (0.5 mL) was added to the above mixture at 23° C. The resulting solution was stirred at 23° C. for 2 hr under nitrogen. The mixture was dissolved in acetonitrile (2 mL) and was purified by prep-HPLC with the following condition: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 30% B in 8 min, 30% B; Wave Length: 254 nm; RT1 (min): 7.5) to afford 2′-chloro-3′-fluoro-5′-methoxy-6-methyl-N-(5-(oxetan-3-yloxy)-1,3,4-thiadiazol-2-yl)-(4,4′-bipyridine)-3-carboxamide (56.0 mg, 36% yield) as a white solid. MS (ESI) calc'd for (C₁₈H₁₅ClFN₅O₄S) (M+1)⁺, 452.1. found 452.1. ¹H NMR (400 MHz, DMSO-d₆) δ 13.15 (s, 1H), 8.98 (s, 1H), 8.18 (s, 1H), 7.46 (s, 1H), 5.75-5.62 (m, 1H), 4.92-4.78 (m, 2H), 4.68-4.52 (m, 2H), 3.74 (s, 3H), 2.59 (s, 3H).

### Example 61

### 3′-fluoro-5′-methoxy-N-(5-methoxy-1,3,4-thiadiazol-2-yl)-2′,6-dimethyl-(4,4′-bipyridine)-3-carboxamide

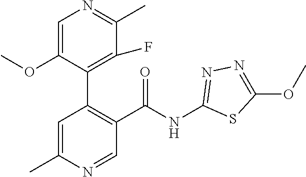

### Step 1 methyl 3′-fluoro-5′-methoxy-2′,6-dimethyl-(4,4′-bipyridine)-3-carboxylate

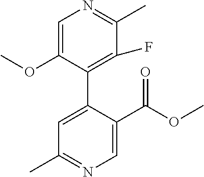

To a stirred solution of methyl 2′-chloro-3′-fluoro-5′-methoxy-6-methyl-(4,4′-bipyridine)-3-carboxylate (800.0 mg, 2.57 mmol) in 1,4-dioxane (8 mL) were sequentially added Trimethylboroxine (388.0 mg, 3.09 mmol), (1,1′-Bis(diphenylphosphino)ferrocene)dichloropalladium(II) (188.0 mg, 0.26 mmol) and potassium carbonate (1.0 g, 7.72 mmol) at 23° C. The resulting solution was stirred at 100° C. for 2 hr under nitrogen. The reaction solution was diluted with ethyl acetate, and the suspension was filtered. The filtrate was collected and concentrated under vacuum. The resulting residue was dissolved in dichloromethane (3 mL) which was applied to a 80.0 C18 column and purified by Combi Flash (Biotage Isolera Prime), eluted with 5˜45% acetonitrile in water within 30 min to afford methyl 3′-fluoro-5′-methoxy-2′,6-dimethyl-(4,4′-bipyridine)-3-carboxylate (268.0 mg, 32% yield) as a brown oil. MS (ESI) calc'd for (C₁₅H₁₅FN₂O₃) (M+1)⁺, 291.1. found 291.1.

### Step 2 3′-fluoro-5′-methoxy-2′,6-dimethyl-(4,4′-bipyridine)-3-carboxylic Acid

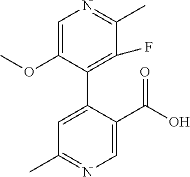 [SMILES: COc1cnc(C)c(F)c1-c1cc(C)ncc1C(=O)O]

To a stirred solution of methyl 3′-fluoro-5′-methoxy-2′,6-dimethyl-(4,4′-bipyridine)-3-carboxylate (260.0 mg, 0.90 mmol) in methanol (1.5 mL) were added water (1.5 mL) and Sodium hydroxide (143.0 mg, 3.58 mmol) at 23° C. The resulting solution was stirred at 23° C. for 1 hr under nitrogen. The organic solvent was removed under vacuum. The aqueous layer was acidified with sat. citric acid solution to pH ˜6. The resulting mixture was dissolved in acetonitrile (2 mL) which was applied to a 20 g C18 column and purified by Combi Flash (Biotage Isolera Prime), eluted with 0˜10% acetonitrile in water within 30 min to afford 3′-fluoro-5′-methoxy-2′,6-dimethyl-(4,4′-bipyridine)-3-carboxylic acid (168.0 mg, 64% yield) as a white solid. MS (ESI) calc'd for (C₁₄H₁₃FN₂O₃) (M+1)⁺, 277.1. found, 277.1.

### Step 3 3′-fluoro-5′-methoxy-N-(5-methoxy-1,3,4-thiadiazol-2-yl)-2′,6-dimethyl-(4,4′-bipyridine)-3-carboxamide

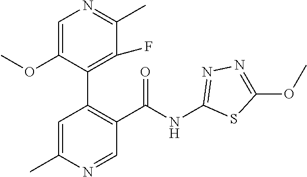

To a stirred solution of 3′-fluoro-5′-methoxy-2′,6-dimethyl-(4,4′-bipyridine)-3-carboxylic acid (140.0 mg, 0.51 mmol) and 5-methoxy-1,3,4-thiadiazol-2-amine (67.0 mg, 0.51 mmol) in acetonitrile (1 mL) was added 1-methylimidazole (166.5 mg, 2.03 mmol). Then TCFH (142.0 mg, 0.51 mmol) in acetonitrile (1 mL) was added to the above mixture at 23° C. The resulting solution was stirred at 23° C. for 2 hr under nitrogen. The resulting residue was dissolved in acetonitrile (5 mL) which was applied to a 80 g C18 column and purified by Combi Flash (Biotage Isolera Prime), eluted with 5˜43% acetonitrile in water within 30 min to afford 3′-fluoro-5′-methoxy-N-(5-methoxy-1,3,4-thiadiazol-2-yl)-2′,6-dimethyl-(4,4′-bipyridine)-3-carboxamide (85.0 mg, 43% yield) as a white solid. MS (ESI) calc'd for (C₁₇H₁₆FN₅O₃S) (M+1)⁺, 390.1. found 390.2. ¹H NMR (400 MHz, DMSO-d₆) δ 12.98 (s, 1H), 8.91 (s, 1H), 8.17 (s, 1H), 7.39 (s, 1H), 4.07 (s, 3H), 3.68 (s, 3H), 2.59 (s, 3H), 2.43 (s, 3H).

### Example 62

### 2′-chloro-5′-(difluoromethoxy)-6-methyl-N-(5-(oxetan-3-yloxy)-1,3,4-thiadiazol-2-yl)-(4,4′-bipyridine)-3-carboxamide

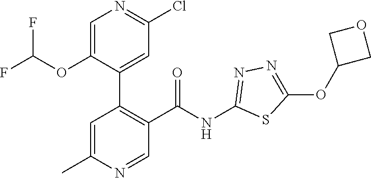 [SMILES: Cc1cc(-c2cc(Cl)ncc2OC(F)F)c(C(=O)Nc2nnc(OC3COC3)s2)cn1]

### Step 1 2-chloro-5-(difluoromethoxy)-4-iodopyridine

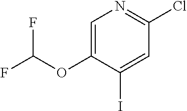 [SMILES: FC(F)Oc1cnc(Cl)cc1I]

To a stirred solution of 6-chloro-4-iodopyridin-3-ol (2.0 g, 7.83 mmol) in N, N-Dimethylformamide (20 mL) were added sodium 2-chloro-2,2-difluoroacetate (2.4 g, 15.68 mmol) and Cs₂CO₃ (3.3 g, 10.18 mmol) at 25° C. The resulting solution was stirred at 80° C. for 2 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (4 mL) and purified by Combi Flash (Biotage Isolera Prime) which applied to 80 g silica gel column that was eluted with 0˜21% ethyl acetate in petroleum ether within 30 min to afford 2-chloro-5-(difluoromethoxy)-4-iodopyridine (1.9 g, 76%) as a white solid. MS (ESI) calculated for (C₆H₃ClF₂INO) (M+1)⁺, 305.0. found, 305.7.

### Step 2 methyl 2′-chloro-5′-(difluoromethoxy)-6-methyl-(4,4′-bipyridine)-3-carboxylate

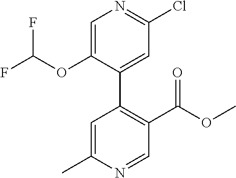 [SMILES: COC(=O)c1cnc(C)cc1-c1cc(Cl)ncc1OC(F)F]

To a degassed solution of methyl 6-methyl-4-(4,4,5,5-tetramethyl-1,3,2-dioxaborolan-2-yl)nicotinate (2.3 g, 8.44 mmol) and 2-chloro-5-(difluoromethoxy)-4-iodopyridine (1.9 g, 6.22 mmol) in 1,4-Dioxane (9 mL) and water (1.8 mL) were added K₂CO₃ (2.3 g, 16.93 mmol) and (1,1′-Bis(diphenylphosphino)ferrocene)dichloropalladium(II) complex with dichloromethane (1.4 g, 1.69 mmol) at 23° C. under nitrogen atmosphere. The resulting solution was stirred at 80° C. for 2 hr under nitrogen atmosphere. The suspension was filtered. The filtrate was concentrated under vacuum. The resulting residue was dissolved in DCM (10 mL) and purified by Combi Flash (Biotage Isolera Prime) which applied to a 120 g silica gel column that was eluted with 0˜45% ethyl acetate in petroleum ether within 30 min to afford methyl 2′-chloro-5′-(difluoromethoxy)-6-methyl-(4,4′-bipyridine)-3-carboxylate (1.7 g, 82%) as a yellow oil. MS (ESI) calculated for (C₁₄H₁₁ClF₂N₂O₃) (M+1)⁺, 329.0. found, 329.10.

### Step 3 2′-chloro-5′-(difluoromethoxy)-6-methyl-(4,4′-bipyridine)-3-carboxylic Acid

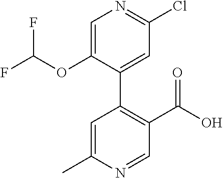 [SMILES: Cc1cc(-c2cc(Cl)ncc2OC(F)F)c(C(=O)O)cn1]

To a stirred solution of methyl 2′-chloro-5′-(difluoromethoxy)-6-methyl-(4,4′-bipyridine)-3-carboxylate (1.7 g, 5.17 mmol) in methanol (5 mL) was added a solution of NaOH (0.83 g, 20.69 mmol) in H₂O (1 mL) at 25° C. The resulting solution was stirred at 25° C. for 1 hr. The organic solvent was removed under vacuum. The aqueous layer was acidified with citric acid to pH ˜6. The resulting mixture was diluted with DMF (2 mL) which was applied to a 80 g C18 column and purified by Combi Flash (Biotage Isolera Prime), eluted with 5˜60% acetonitrile in water within 35 min to afford 2′-chloro-5′-(difluoromethoxy)-6-methyl-(4,4′-bipyridine)-3-carboxylic acid (120.0 mg, 6%) as a white solid. MS (ESI) calculated for (C₁₃H₉ClF₂N₂O₃) (M+1)⁺, 315.0. found, 315.10.

### Step 4 2′-chloro-5′-(difluoromethoxy)-6-methyl-N-(5-(oxetan-3-yloxy)-1,3,4-thiadiazol-2-yl)-(4,4′-bipyridine)-3-carboxamide

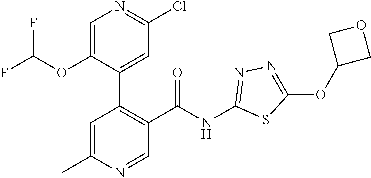 [SMILES: Cc1cc(-c2cc(Cl)ncc2OC(F)F)c(C(=O)Nc2nnc(OC3COC3)s2)cn1]

To a stirred solution of 2′-chloro-5′-(difluoromethoxy)-6-methyl-(4,4′-bipyridine)-3-carboxylic acid (347.0 mg, 1.10 mmol) in acetonitrile (4 mL) were added 5-(oxetan-3-yloxy)-1,3,4-thiadiazol-2-amine (191.0 mg, 1.10 mmol, Example 13, Step 1) and 1-Methylimidazole (452.0 mg, 5.51 mmol) at 23° C. To the above solution was added a solution of TCFH (309.0 mg, 1.10 mmol) in acetonitrile (2 mL) at 23° C. under nitrogen. The resulting mixture was then stirred at 23° C. for 1 hr. The reaction mixture (2 mL) was purified by prep-HPLC with the following condition: (Column: XBridge Prep OBD C18 Column, 30*150 mm, 5 um; Mobile Phase A: Water (10 mmol/L NH₄HCO₃), Mobile Phase B: ACN; Flow rate: 60 mL/min; Gradient: 21% B to 31% B in 8 min, 31% B to 95% B in 8.2 min, 95% B to 95% B in 9.5 min, 95% B to 5% B in 11 min, 5% B; Wave Length: 254 nm; RT1 (min): 7; Injection Volume: 0.5 mL; Number Of Runs: 3) to afford 2′-chloro-5′-(difluoromethoxy)-6-methyl-N-(5-(oxetan-3-yloxy)-1,3,4-thiadiazol-2-yl)-(4,4′-bipyridine)-3-carboxamide (52.9 mg, 10%) as a white solid. MS (ESI) calculated for (C₁₈H₁₄ClF₂N₅O₄S) (M+1)⁺, 470. found, 470.15. ¹H NMR (400 MHz, DMSO-d₆) δ 13.19 (s, 1H), 8.96 (s, 1H), 8.34 (s, 1H), 7.73 (s, 1H), 7.46 (s, 1H), 7.32-6.82 (m, 1H), 5.70-5.64 (m, 1H), 4.92-4.88 (m, 2H), 4.66-4.62 (m, 2H), 2.68 (s, 3H).

### Example 63

### 2′-bromo-5′-methoxy-N-(5-methoxy-1,3,4-thiadiazol-2-yl)-6-methyl-(4,4′-bipyridine)-3-carboxamide

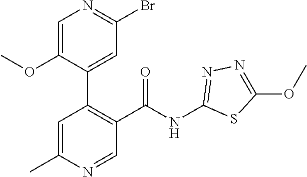

### Step 1 2-bromo-4-iodo-5-methoxypyridine

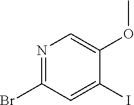

To a stirred solution of methanol (380.0 mg, 11.84 mmol) in N,N-Dimethylformamide (15 mL) was added NaH (230.0 mg, 5.68 mmol) at 0° C. The reaction mixture was stirred at 0° C. for 0.5 h. Then 2-bromo-5-fluoro-4-iodopyridine (1.4 g, 4.74 mmol) was added to the above mixture at 0° C. The resulting solution was then stirred at 25° C. for 1 h. The reaction mixture was quenched by the addition of water and extracted with ethyl acetate. The combined organic layers were washed with brine, dried over anhydrous sodium sulfate, filtered, and concentrated under vacuum. The resulting residue was dissolved in dichloromethane (5 mL) and purified by Combi Flash (Biotage Isolera Prime) which applied to a 80.0 g silica gel column that was eluted with 0˜20% ethyl acetate in petroleum ether within 25 min to afford 2-bromo-4-iodo-5-methoxypyridine (1.2 g, 54% yield) as a white solid. MS (ESI) calc'd for (C₆H₅BrINO) (M+1)⁺, 313.9. found 313.9.

### Step 2 methyl 2′-bromo-5′-methoxy-6-methyl-(4,4′-bipyridine)-3-carboxylate

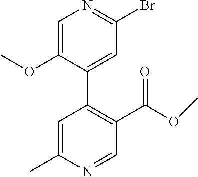

To a stirred solution of 2-bromo-4-iodo-5-methoxypyridine (200.0 mg, 0.64 mmol) and methyl 6-methyl-4-(4,4,5,5-tetramethyl-1,3,2-dioxaborolan-2-yl)nicotinate (265.0 mg, 0.96 mmol) in 1,4-dioxane (2 mL) were sequentially added water (0.4 mL), potassium carbonate (264.0 mg, 1.91 mmol) and 1, l′-bis(diphenylphosphino)ferrocene-palladium(II)dichloride dichloromethane complex (52.0 mg, 0.06 mmol) at 23° C. The resulting solution was stirred at 80° C. for 2 hr under nitrogen. The suspension was filtered. The filtrate was collected and concentrated under vacuum. The resulting residue was dissolved in acetonitrile (3 mL) which was applied to a 40.0 g C18 column and purified by Combi Flash (Biotage Isolera Prime), eluted with 5˜45% acetonitrile in water within 30 min to afford methyl 2′-bromo-5′-methoxy-6-methyl-(4,4′-bipyridine)-3-carboxylate (80.0 mg, 36% yield) as a colorless oil. MS (ESI) calc'd for (C₁₄H₁₃BrN₂O₃) (M+1)⁺, 337.0. found 337.0.

### Step 3 2′-bromo-5′-methoxy-6-methyl-(4,4′-bipyridine)-3-carboxylic Acid

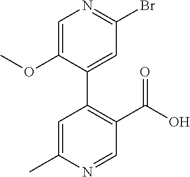 [SMILES: COc1cnc(Br)cc1-c1cc(C)ncc1C(=O)O]

To a stirred solution of methyl 2′-bromo-5′-methoxy-6-methyl-(4,4′-bipyridine)-3-carboxylate (175.0 mg, 0.52 mmol) in methanol (0.3 mL) were added water (0.3 mL) and sodium hydroxide (83.0 mg, 2.08 mmol) at 25° C. The resulting solution was stirred at 25° C. for 2 hr under nitrogen. The organic solvent was removed under vacuum. The aqueous layer was acidified with sat. citric acid solution to pH ˜6 and extracted with ethyl acetate. The combined organic layers were washed with brine, dried over anhydrous sodium sulfate, filtered, and concentrated under vacuum to afford 2′-bromo-5′-methoxy-6-methyl-(4,4′-bipyridine)-3-carboxylic acid (100.0 mg, 58% yield) as a yellow solid. MS (ESI) calc'd for (C₁₃H₁₁BrN₂O₃) (M+1)⁺, 323.0. found 323.1.

### Step 4 2′-bromo-5′-methoxy-N-(5-methoxy-1,3,4-thiadiazol-2-yl)-6-methyl-(4,4′-bipyridine)-3-carboxamide

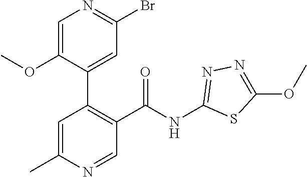

To a stirred solution of 2′-bromo-5′-methoxy-6-methyl-(4,4′-bipyridine)-3-carboxylic acid (100.0 mg, 0.31 mmol) and 5-methoxy-1,3,4-thiadiazol-2-amine (40.0 mg, 0.31 mmol) in acetonitrile (1 mL) was added 1-methylimidazole (0.1 mL, 1.24 mmol). Then TCFH (87.0 mg, 0.31 mmol) in acetonitrile (1 mL) was added to the above mixture at 23° C. The resulting solution was stirred at 23° C. for 2 hr under nitrogen. The reaction mixture was diluted with DMF (2 mL) and was purified by prep-HPLC with the following condition: (Column: XBridge Prep OBD C18 Column, 19*250 mm, 5 um; Mobile Phase A: Water (10 mmol/L NH₄HCO₃), Mobile Phase B: MeOH—HPLC; Flow rate: 25 mL/min; Gradient: 50% B to 55% B in 10 min, 55% B to 95% B in 10.2 min, 95% B to 95% B in 12 min, 95% B to 5% B in 12.2 min, 5% B to 5% B in 14 min; Wave Length: 254 nm; RT1 (min): 9; Injection Volume: 0.6 mL; Number Of Runs: 4) to afford 2′-bromo-5′-methoxy-N-(5-methoxy-1,3,4-thiadiazol-2-yl)-6-methyl-(4,4′-bipyridine)-3-carboxamide (45.0 mg, 33% yield) as a white solid. MS (ESI) calc'd for (C₁₆H₁₄BrN₅O₃S) (M+1)⁺, 436.0. found 436.2. ¹H NMR (400 MHz, DMSO-d₆) δ 12.90 (s, 1H), 8.82 (s, 1H), 8.17 (s, 1H), 7.62 (s, 1H), 7.41 (s, 1H), 4.07 (s, 3H), 3.62 (s, 3H), 2.59 (s, 3H).

### Example 64

### 2′-chloro-6-(hydroxymethyl)-5′-methoxy-N-(5-methoxy-1,3,4-thiadiazol-2-yl)-(4,4′-bipyridine)-3-carboxamide

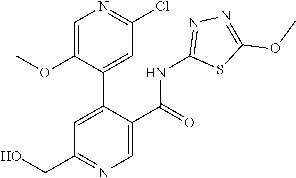

### Step 1 2′-chloro-6-(hydroxymethyl)-5′-methoxy-(4,4′-bipyridine)-3-carboxylic Acid

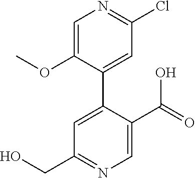 [SMILES: COc1cnc(Cl)cc1-c1cc(CO)ncc1C(=O)O]

To a solution of ethyl 2′-chloro-6-(hydroxymethyl)-5′-methoxy-(4,4′-bipyridine)-3-carboxylate (300.0 mg, 0.93 mmol) in Tetrahydrofuran (1.5 mL) were added LiOH (66.8 mg, 2.79 mmol) and Water (0.5 mL) at 18° C. The resulting solution was stirred at 50° C. for 16 hr. The reaction mixture was diluted with water, acidified with citric acid to pH 4˜5 and extracted with ethyl acetate. The combined organic layers were washed with brine, dried over anhydrous sodium sulfate, filtered, and concentrated under vacuum to afford 2′-chloro-6-(hydroxymethyl)-5′-methoxy-(4,4′-bipyridine)-3-carboxylic acid (120.0 mg, 39%) as a brown solid. MS (ESI) calc'd for (C₁₃H₁₁ClN₂O₄) (M+1)⁺, 295.0. found 295.0.

### Step 2 2′-chloro-6-(hydroxymethyl)-5′-methoxy-N-(5-methoxy-1,3,4-thiadiazol-2-yl)-(4,4′-bipyridine)-3-carboxamide

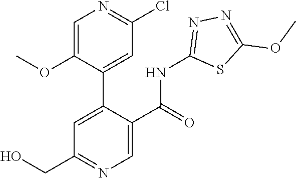

To a mixture of 2′-chloro-6-(hydroxymethyl)-5′-methoxy-(4,4′-bipyridine)-3-carboxylic acid (250.0 mg, 0.76 mmol) in Acetonitrile (3 mL) were added 5-methoxy-1,3,4-thiadiazol-2-amine (120.0 mg, 0.91 mmol) and 1-methyl-1H-imidazole (188.0 mg, 2.21 mmol). A solution of TCFH (321.0 mg, 1.14 mmol) in Acetonitrile (1 mL) was added thereto dropwise under nitrogen. The resulting solution was stirred at 18° C. for 2 hr. The solvent was removed under vacuum. The residue was dissolved in DMF (3 mL) and was purified by prep-HPLC with the following condition: (Column: Xselect CSH C₁₈ OBD Column 30*150 mm 5 μm; Mobile Phase A: Water (0.1% FA), Mobile Phase B: ACN; Flow rate: 60 mL/min; Gradient: 20% B to 30% B in 8 min, 30% B to 95% B in 8.2 min, 95% B to 95% B in 9.7 min, 95% B to 5% B in 11 min, 5% B; Wave Length: 220 nm; RT1 (min): 6.68; Injection Volume: 0.8 mL; Number Of Runs: 3) to afford 2′-chloro-6-(hydroxymethyl)-5′-methoxy-N-(5-methoxy-1,3,4-thiadiazol-2-yl)-(4,4′-bipyridine)-3-carboxamide (75.2 mg, 24%) as a white solid MS (ESI) calc'd for (C₁₆H₁₄ClN₅O₄S) (M+1)⁺, 408.0. found 408.0. ¹H NMR (400 MHz, DMSO-d₆) δ 12.94 (s, 1H), 8.87 (s, 1H), 8.19 (s, 1H), 7.51 (s, 2H), 5.59 (t, J=5.6 Hz, 1H), 4.67 (d, J=5.6 Hz, 2H), 4.08 (s, 3H), 3.64 (s, 3H).

### Example 65

### 2′-chloro-5′-methoxy-5-((5-methoxy-1,3,4-thiadiazol-2-yl)carbamoyl)-2-methyl-(4,4′-bipyridine) 1-oxide

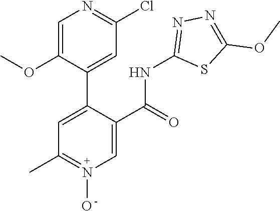

### Step 1 methyl 4-chloro-6-methylnicotinate

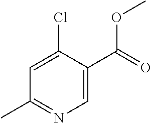

To a stirred solution of 4-chloro-6-methylnicotinic acid (10.0 g, 58.30 mmol) and potassium carbonate (24.1 g, 175.00 mmol) in N,N-Dimethylformamide (DMF) (150 mL) was added iodomethane (5.47 mL, 87.00 mmol) at 20° C. The resulting solution was stirred at 20° C. for 16 hr. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to a 120 g silica gel column that was eluted with 0˜60% ethyl acetate in petroleum ether within 30 min to afford methyl 4-chloro-6-methylnicotinate (8.5 g, 78%) as a yellow oil. MS (ESI) calc'd for (C₈H₈ClNO₂) (M+1)⁺, 186.0. found 186.0.

### Step 2 4-chloro-5-(methoxycarbonyl)-2-methylpyridine 1-oxide

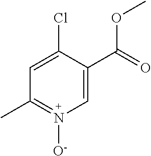

To a stirred solution of methyl 4-chloro-6-methylnicotinate (1.0 g, 5.39 mmol) in Dichloromethane (15 mL) was added mCPBA (1.3 g, 6.47 mmol). The mixture was stirred at 25° C. for 2 hr. The reaction mixture was quenched by the addition of water and extracted with dichloromethane. The combined organic layers were washed with saturated sodium thiosulfate solution, saturated sodium carbonate solution and brine. The combined organic layers were dried over anhydrous sodium sulfate, filtered, and concentrated under vacuum. The resulting residue was dissolved in ACN (6 mL) which was applied to a 80 g C18 column and purified by Combi Flash (Biotage Isolera Prime), eluted with 5˜18% acetonitrile in water within 22 min to afford 4-chloro-5-(methoxycarbonyl)-2-methylpyridine 1-oxide (870.0 mg, 78%) as a white solid. MS (ESI) calc'd for (C₈H₈ClNO₃) (M+1)⁺, 202.0. found 202.0.

### Step 3 2′-chloro-5′-methoxy-5-(methoxycarbonyl)-2-methyl-(4,4′-bipyridine) 1-oxide

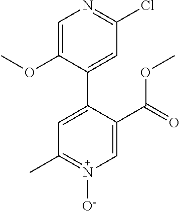

To a stirred solution of 4-chloro-5-(methoxycarbonyl)-2-methylpyridine 1-oxide (600.0 mg, 2.98 mmol) in 1,4-Dioxane (5 mL) and Water (1.7 mL) under nitrogen atmosphere were added (2-chloro-5-methoxypyridin-4-yl)boronic acid (837.0 mg, 4.46 mmol), K₂CO₃ (1234.0 mg, 8.93 mmol) and (1,1′-Bis(di-tert-butylphosphino)ferrocene)dichlor (194.0 mg, 0.29 mmol) at 25° C. The reaction mixture was stirred at 80° C. for 2 hr under nitrogen atmosphere before filtered. The filtrate was collected and concentrated under vacuum. The resulting residue was dissolved in DMF (5 mL) was applied to a 80 g C18 column and purified by Combi Flash (Biotage Isolera Prime), eluted with 5˜28% acetonitrile in water within 30 min to afford 2′-chloro-5′-methoxy-5-(methoxycarbonyl)-2-methyl-(4,4′-bipyridine) 1-oxide (500.0 mg, 53%) as a yellow solid. MS (ESI) calc'd for (C₁₄H₁₃ClN₂O₄) (M+1)⁺, 309.0. found 309.0.

### Step 4 5-carboxy-2′-chloro-5′-methoxy-2-methyl-(4,4′-bipyridine) 1-oxide

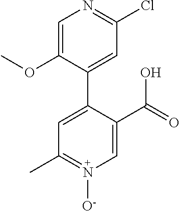

To a stirred solution of 2′-chloro-5′-methoxy-5-(methoxycarbonyl)-2-methyl-(4,4′-bipyridine) 1-oxide (250.0 mg, 0.81 mmol) in Methanol (1.5 mL) and Water (1.5 mL) was added NaOH (130.0 mg, 3.24 mmol) at 25° C. The resulting solution was stirred at 25° C. for 2 hr. The mixture was acidified with citric acid to pH ˜6, applied to a 40 g C18 column and purified by Combi Flash (Biotage Isolera Prime), eluted with 5˜34% acetonitrile in water within 20 min to afford 5-carboxy-2′-chloro-5′-methoxy-2-methyl-(4,4′-bipyridine) 1-oxide (220.0 mg, 90%) as a white solid. MS (ESI) calc'd for (C₁₃H₁₁ClN₂O₄) (M+1)⁺, 295.0. found 295.0.

### Step 5 2′-chloro-5′-methoxy-5-((5-methoxy-1,3,4-thiadiazol-2-yl)carbamoyl)-2-methyl-(4,4′-bipyridine) 1-oxide

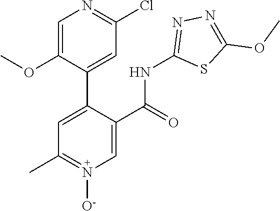

To a stirred solution of 5-carboxy-2′-chloro-5′-methoxy-2-methyl-(4,4′-bipyridine) 1-oxide (180.0 mg, 0.61 mmol) and 5-methoxy-1,3,4-thiadiazol-2-amine (80.0 mg, 0.61 mmol) in Acetonitrile (3 mL) were added 1-methyl-1H-imidazole (251.0 mg, 3.05 mmol). A solution of TCFH (172.0 mg, 0.61 mmol) in Acetonitrile (1 mL) was added thereto at 25° C. The mixture was stirred at 25° C. for 3 hr. The resulting mixture (5 mL) was applied to a 80 g C18 column and purified by Combi Flash (Biotage Isolera Prime), eluted with 5˜51% acetonitrile in water within 30 min to afford an off-white solid (85%), which was further purified by prep-HPLC with the following condition: (Column: Xselect CSH F-Phenyl OBD column, 19*250 mm, 5 μm; Mobile Phase A: Water (10 mmol/L NH₄HCO₃), Mobile Phase B: MeOH—HPLC; Flow rate: 25 mL/min; Gradient: 20% B to 50% B in 10 min, 50% B; Wave Length: 254 nm; RT1 (min): 10) to afford 2′-chloro-5′-methoxy-5-((5-methoxy-1,3,4-thiadiazol-2-yl)carbamoyl)-2-methyl-(4,4′-bipyridine) 1-oxide (80.0 mg, 32%) as a white solid. MS (ESI) calc'd for (C₁₆H₁₄ClN₅O₄S) (M+1)⁺, 408.0. found 408.1. ¹H NMR (400 MHz, DMSO-d₆) δ 13.03 (s, 1H), 8.63 (s, 1H), 8.15 (s, 1H), 7.69 (s, 1H), 7.54 (s, 1H), 4.07 (s, 3H), 3.62 (s, 3H), 2.44 (s, 3H).

### Example 66

### 4-(5-chloro-2-(hydroxymethyl)phenyl)-N-(5-methoxy-1,3,4-thiadiazol-2-yl)-6-methylnicotinamide

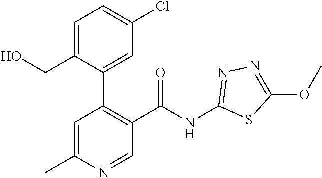 [SMILES: COc1nnc(NC(=O)c2cnc(C)cc2-c2cc(Cl)ccc2CO)s1]

### Step 1 tert-butyl 4-chloro-6-methylnicotinate

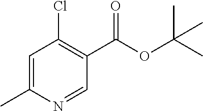

To a stirred solution of 4-chloro-6-methylnicotinic acid (5.0 g, 29.10 mmol) in Dichloromethane (50 mL) were added di-tert-butyl dicarbonate (9.5 g, 43.70 mmol) and DMAP (356.1 mg, 2.91 mmol) at 0° C. The resulting solution was stirred at 23° C. for 16 h. The organic solvent was removed under vacuum. The resulting residue was dissolved in DCM (10 mL) and purified by Combi Flash (Biotage Isolera Prime) which applied to a 120 g silica gel column that was eluted with 0˜30% ethyl acetate in petroleum ether within 35 min to afford tert-butyl 4-chloro-6-methylnicotinate (3.0 g, 45%) as a colorless oil. MS (ESI) calc'd for (C₁₁H₁₄ClNO₂) (M+1)⁺, 228.0. found 228.1.

### Step 2 tert-butyl 6-methyl-4-(4,4,5,5-tetramethyl-1,3,2-dioxaborolan-2-yl)nicotinate

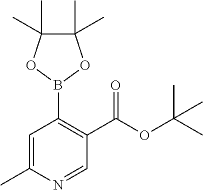 [SMILES: Cc1cc(B2OC(C)(C)C(C)(C)O2)c(C(=O)OC(C)(C)C)cn1]

To a stirred solution of tert-butyl 4-chloro-6-methylnicotinate (3.0 g, 13.18 mmol) in 1,4-Dioxane (50 mL) were added Bis(pinacolato)diboron (16.7 g, 65.90 mmol), AcOK (2.6 g, 26.40 mmol) and Pd(dppf)Cl₂ (1.1 g, 1.32 mmol) at 23° C. The resulting solution was stirred at 100° C. for 16 h under nitrogen. The reaction mixture was diluted with water. The aqueous layer was extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to a 120 g silica gel column that was eluted with 0˜30% ethyl acetate in petroleum ether within 35 min to afford tert-butyl 6-methyl-4-(4,4,5,5-tetramethyl-1,3,2-dioxaborolan-2-yl) nicotinate (4.6 g, 98%) as a colorless oil. MS (ESI) calc'd for (C₁₇H₂₆BNO₄) (M+1)⁺, 320.2. found, 320.2.

### Step 3 tert-butyl 4-(5-chloro-2-(methoxycarbonyl)phenyl)-6-methylnicotinate

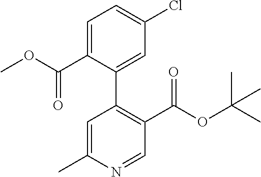 [SMILES: COC(=O)c1ccc(Cl)cc1-c1cc(C)ncc1C(=O)OC(C)(C)C]

To a stirred solution of tert-butyl 6-methyl-4-(4,4,5,5-tetramethyl-1,3,2-dioxaborolan-2-yl) nicotinate (2.0 g, 6.27 mmol) in 1,4-Dioxane (30 mL) were added methyl 4-chloro-2-iodobenzoate (1.8 g, 6.27 mmol), K₂CO₃ (2.6 g, 18.80 mmol) and Water (3 mL) at 23° C. To the above solution was added Pd(dppf)Cl₂ (512.2 mg, 0.63 mmol) at 23° C. under nitrogen. The resulting mixture was stirred at 80° C. for 2 h under nitrogen. The reaction mixture was diluted with water. The aqueous layer was extracted with ethyl acetate. The combined organic layers were washed with brine, dried over anhydrous sodium sulfate, filtered, and concentrated under vacuum. The resulting residue was dissolved in DCM (5 mL) and purified by Combi Flash (Biotage Isolera Prime) which applied to a 80 g silica gel column that was eluted with 0˜40% ethyl acetate in petroleum ether within 25 min to afford tert-butyl 4-(5-chloro-2-(methoxycarbonyl)phenyl)-6-methylnicotinate (2.0 g, 86%) as a colorless oil MS (ESI) calc'd for (C₁₉H₂₀ClNO₄) (M+1)⁺, 362.1. found 362.2.

### Step 4 4-(5-chloro-2-(methoxycarbonyl)phenyl)-6-methylnicotinic Acid

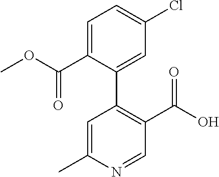 [SMILES: COC(=O)c1ccc(Cl)cc1-c1cc(C)ncc1C(=O)O]

To a stirred solution of tert-butyl 4-(5-chloro-2-(methoxycarbonyl)phenyl)-6-methylnicotinate (500.0 mg, 1.38 mmol) in Dichloromethane (6 mL) was added TFA (3 mL) at 23° C. The resulting solution was stirred at 23° C. for 16 h. The organic solvent was removed under vacuum to afford 4-(5-chloro-2-(methoxycarbonyl)phenyl)-6-methylnicotinic acid TFA salt (425.0 mg, 98%) as a yellow oil. MS (ESI) calc'd for (C₁₅H₁₂ClNO₄) (M+1)⁺, 306.1. found 306.3.

### Step 5 methyl 4-chloro-2-(5-((5-methoxy-1,3,4-thiadiazol-2-yl) carbamoyl)-2-methylpyridin-4-yl)benzoate

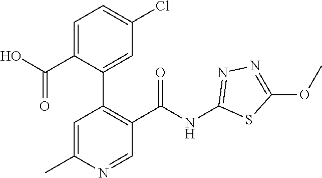 [SMILES: COc1nnc(NC(=O)c2cnc(C)cc2-c2cc(Cl)ccc2C(=O)O)s1]

To a stirred solution of 4-(5-chloro-2-(methoxycarbonyl) phenyl)-6-methylnicotinic acid (420.0 mg, 1.37 mmol) in acetonitrile (5 mL) were added 5-methoxy-1,3,4-thiadiazol-2-amine (180.0 mg, 1.37 mmol) and 1-methylimidazole (564.0 mg, 6.87 mmol). To the above solution was added a solution of TCFH (386.0 mg, 1.37 mmol) in acetonitrile (3 mL). The resulting mixture was stirred at 23° C. for 2 h. The suspension was filtered. The filter cake was collected, washed with acetonitrile and dried under vacuum to afford methyl 4-chloro-2-(5-((5-methoxy-1,3,4-thiadiazol-2-yl) carbamoyl)-2-methylpyridin-4-yl) benzoate (450.0 mg, 76%) as a white solid. MS (ESI) calc'd for (C₁₈H₁₅ClN₄O₄S) (M+1)⁺, 419.0. found 419.0.

### Step 6 4-(5-chloro-2-(hydroxymethyl)phenyl)-N-(5-methoxy-1,3,4-thiadiazol-2-yl)-6-methylnicotinamide

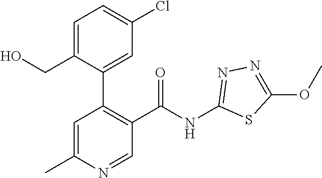 [SMILES: COc1nnc(NC(=O)c2cnc(C)cc2-c2cc(Cl)ccc2CO)s1]

To a stirred solution of methyl 4-chloro-2-(5-((5-methoxy-1,3,4-thiadiazol-2-yl)carbamoyl)-2-methylpyridin-4-yl)benzoate (70.0 mg, 0.17 mmol) in Tetrahydrofuran (1 mL) was added Lithium Aluminum Hydride (31.1 mg, 0.82 mmol) in portions at 0° C. The resulting solution was stirred at 0° C. for 2 h. The reaction mixture was quenched by the addition of water and acidified with citric acid to pH ˜5 and extracted with ethyl acetate. The combined organic layers were washed with brine, dried over anhydrous sodium sulfate, filtered, and concentrated under vacuum. The resulting residue was dissolved in DMF (2 mL) which was applied to a 20 g C18 column and purified by Combi Flash (Biotage Isolera Prime), eluted with 5˜50% acetonitrile in water within 25 min to afford 4-(5-chloro-2-(hydroxymethyl)phenyl)-N-(5-methoxy-1,3,4-thiadiazol-2-yl)-6-methylnicotinamide (26.6 mg, 40%) as a white solid. MS (ESI) calc'd for (C₁₇H₁₅ClN₄O₃S) (M+1)⁺, 391.1. found 391.1. 1H NMR (400 MHz, DMSO-d₆) δ 12.79 (s, 1H), 8.82 (s, 1H), 7.54 (d, J=8.4 Hz, 1H), 7.46 (dd, J=8.4, 2.4 Hz, 1H), 7.30 (s, 1H), 7.15 (d, J=2.4 Hz, 1H), 5.37 (br, 1H), 4.23 (s, 2H), 4.05 (s, 3H), 2.58 (s, 3H).

### Example 67

### N-(5-methoxy-1,3,4-thiadiazol-2-yl)-4-(5-methoxy-1H-indazol-6-yl)-6-methylnicotinamide

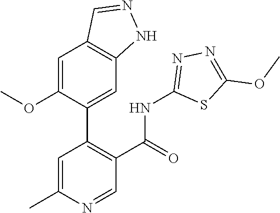

### Step 1 methyl 4-(5-methoxy-1H-indazol-6-yl)-6-methylnicotinate

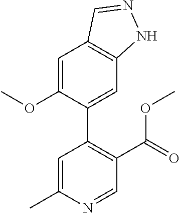

To a stirred solution of 6-bromo-5-methoxy-1H-indazole (500.0 mg, 2.20 mmol) in 1,4-Dioxane (5 mL) was added methyl 6-methyl-4-(4,4,5,5-tetramethyl-1,3,2-dioxaborolan-2-yl)nicotinate (915.1 mg, 3.30 mmol) at 20° C. To the above solution were sequentially added K₂CO₃ (913.2 mg, 6.61 mmol), Water (1 mL) and PdCl₂(dtbpf) (143.2 mg, 0.22 mmol) at 20 ºC. The resulting mixture was then stirred at 80° C. for 2 hr under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (5 mL) and purified by Combi Flash (Biotage Isolera Prime) which applied to a 40 g silica gel column that was eluted with 0˜20% methanol in dichloromethane within 25 min to afford methyl 4-(5-methoxy-1H-indazol-6-yl)-6-methylnicotinate (341.0 mg, 51%) as a yellow solid. MS (ESI) calc'd for (C₁₆H₁₅N₃O₃) (M+1)⁺, 298.1. found: 298.0.

### Step 2 4-(5-methoxy-1H-indazol-6-yl)-6-methylnicotinic Acid

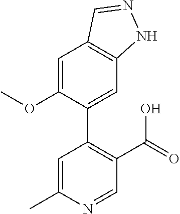 [SMILES: COc1cc2cn[nH]c2cc1-c1cc(C)ncc1C(=O)O]

To a stirred solution of methyl 4-(5-methoxy-1H-indazol-6-yl)-6-methylnicotinate (300.0 mg, 1.00 mmol) in Tetrahydrofuran (THF) (1.5 mL) were added Water (1.5 mL) and LiOH (48.3 mg, 2.01 mmol) at 20° C. The resulting solution was stirred at 20° C. for 6 hr. The mixture was diluted with water (5 mL). The aqueous layer was acidified with citric acid to pH ˜3. The suspension was filtered. The filter cake was washed with water, dried over vacuum to afford 4-(5-methoxy-1H-indazol-6-yl)-6-methylnicotinic acid (54.0 mg, crude) as a white solid. MS (ESI) calc'd for (C₁₅H₁₃N₃O₃) (M+1)⁺, 284.1. found: 284.0.

### Step 3 N-(5-methoxy-1,3,4-thiadiazol-2-yl)-4-(5-methoxy-1H-indazol-6-yl)-6-methylnicotinamide

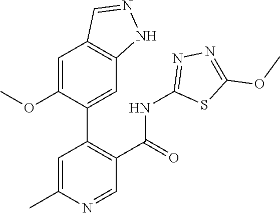

To a stirred solution of 4-(5-methoxy-1H-indazol-6-yl)-6-methylnicotinic acid (54.0 mg, 0.19 mmol) in Acetonitrile (2 mL) were added 5-methoxy-1,3,4-thiadiazol-2-amine (25.0 mg, 0.19 mmol) and 1-methyl-1H-imidazole (78.0 mg, 0.95 mmol) at 20° C. To the above solution was added a solution of TCFH (53.6 mg, 0.19 mmol) in Acetonitrile (2 mL) at 20° C. under nitrogen. The resulting mixture was then stirred at 20° C. for 2 hr under nitrogen. The mixture was diluted with acetonitrile (2 mL) which was applied to a 20 g C18 column and purified by Combi Flash (Biotage Isolera Prime), eluted with 5˜100% acetonitrile in water within 25 min to afford N-(5-methoxy-1,3,4-thiadiazol-2-yl)-4-(5-methoxy-1H-indazol-6-yl)-6-methylnicotinamide (68.6 mg, 88%) as a yellow solid. MS (ESI) calc'd for (C₁₈H₁₆N₆O₃S) (M+1)⁺, 397.1. found: 397.1. ¹H NMR (400 MHz, DMSO-d₆) δ 13.11 (s, 1H), 12.70 (s, 1H), 8.73 (s, 1H), 8.00 (s, 1H), 7.50-7.45 (m, 2H), 7.18 (s, 1H), 4.06 (s, 3H), 3.49 (s, 3H) 2.62 (s, 3H).

### Example 68 and 69

### (R)-2′-(1-hydroxyethyl)-5′-methoxy-N-(5-methoxy-1,3,4-thiadiazol-2-yl)-6-methyl-(4,4′-bipyridine)-3-carboxamide and (S)-2′-(1-hydroxyethyl)-5′-methoxy-N-(5-methoxy-1,3,4-thiadiazol-2-yl)-6-methyl-(4,4′-bipyridine)-3-carboxamide

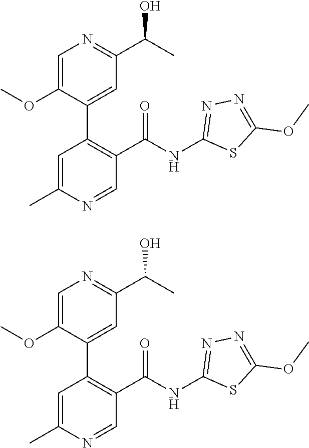

### Step 1 methyl 2′-acetyl-5′-methoxy-6-methyl-(4,4′-bipyridine)-3-carboxylate

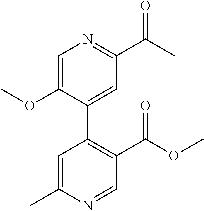

To a stirred solution of methyl 2′-chloro-5′-methoxy-6-methyl-(4,4′-bipyridine)-3-carboxylate (1.5 g, 5.13 mmol) in Toluene (15 mL) were added bis(triphenylphosphine)palladium(II) chloride (3.6 g, 5.12 mmol) and tributyl(1-ethoxyvinyl)stannane (3.7 g, 10.26 mmol) at 25 ºC. The resulting solution was stirred at 100° C. for 4 hr. The organic solvent was removed under vacuum. The residue was dissolved with Methanol (10 mL) and HCl (2 mL, conc.) at 25° C. The resulting mixture was stirred at 25° C. for 1 hr. The residue was diluted with water. The aqueous layer was basified with sat. NaHCO₃ aqueous to pH 7˜8 and extracted with ethyl acetate. The combined organic layers were washed with brine, dried over anhydrous sodium sulfate, filtered, and concentrated under vacuum. The resulting residue was dissolved in DCM (4 mL) and purified by Combi Flash (Biotage Isolera Prime) which applied to a 40 g silica gel column that was eluted with 0˜9% methanol in dichloromethane within 30 min to afford methyl 2′-acetyl-5′-methoxy-6-methyl-(4,4′-bipyridine)-3-carboxylate (680.0 mg, 58%) as a white solid. MS (ESI) calc'd for (C₁₆H₁₆N₂O₄) (M+1)⁺, 301.1. found, 301.1.

### Step 2 2′-acetyl-5′-methoxy-6-methyl-(4,4′-bipyridine)-3-carboxylic Acid

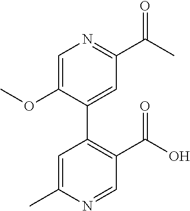 [SMILES: COc1cnc(C(C)=O)cc1-c1cc(C)ncc1C(=O)O]

To a stirred solution of methyl 2′-acetyl-5′-methoxy-6-methyl-(4,4′-bipyridine)-3-carboxylate (650.0 mg, 2.16 mmol) in Methanol (3 mL) were added NaOH (325.0 mg, 8.12 mmol) and Water (3 mL) at 25° C. The resulting solution was stirred at 25° C. for 16 hr before diluted with water. The organic solvent was removed under vacuum. The aqueous layer was acidified with citric acid to pH 5˜6 and extracted with ethyl acetate. The combined organic layers were washed with brine, dried over anhydrous sodium sulfate, filtered, and concentrated under vacuum to afford 2′-acetyl-5′-methoxy-6-methyl-(4,4′-bipyridine)-3-carboxylic acid (550.0 mg, crude) as a white solid. MS (ESI) calc'd for (C₁₅H₁₄N₂O₄) (M+1)⁺, 287.1. found, 287.1.

### Step 3 2′-acetyl-5′-methoxy-N-(5-methoxy-1,3,4-thiadiazol-2-yl)-6-methyl-(4,4′-bipyridine)-3-carboxamide

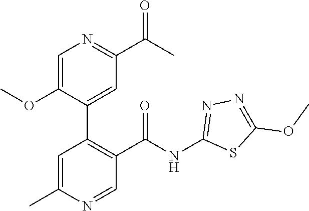

To a stirred solution of 2′-acetyl-5′-methoxy-6-methyl-(4,4′-bipyridine)-3-carboxylic acid (530.0 mg, 1.82 mmol) and 5-methoxy-1,3,4-thiadiazol-2-amine (241.0 mg, 1.82 mmol) in Acetonitrile (4 mL) was added 1-methyl-1H-imidazole (759.0 mg, 9.14 mmol) at 25° C. To the above was added a solution of N-(chloro(dimethylamino)methylene)-N-methylmethanaminium hexafluorophosphate(V) (518.0 mg, 1.82 mmol) in Acetonitrile (2 mL) at 25° C. under nitrogen. The resulting solution was stirred at 25° C. for 2 hr. NaOH (0.3 mL, 1 N) was added thereto at 25° C. The resulting mixture was stirred at 25° C. for 10 min, and then acidified with citric acid to pH 5˜6. The volatiles were removed under vacuum. The resulting residue was dissolved in DMF (3 mL) which was applied to a 40 g C18 column and purified by Combi Flash (Biotage Isolera Prime), eluted with 5˜40% acetonitrile in water within 30 min to afford 2′-acetyl-5′-methoxy-N-(5-methoxy-1,3,4-thiadiazol-2-yl)-6-methyl-(4,4′-bipyridine)-3-carboxamide (450.0 mg, 40%) as a white solid. MS (ESI) calc'd for (C₁₈H₁₇N₅O₄S) (M+1)⁺, 400.1. found, 400.1.

### Step 4 2′-(1-hydroxyethyl)-5′-methoxy-N-(5-methoxy-1,3,4-thiadiazol-2-yl)-6-methyl-(4,4′-bipyridine)-3-carboxamide

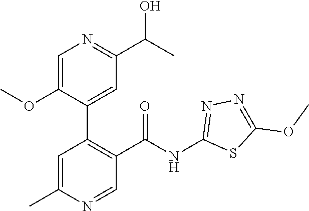

To a stirred solution of 2′-acetyl-5′-methoxy-N-(5-methoxy-1,3,4-thiadiazol-2-yl)-6-methyl-(4,4′-bipyridine)-3-carboxamide (450.0 mg, 1.12 mmol) in Methanol (4 mL) was added NaBH₄ (85.0 mg, 2.25 mmol) at 25° C. The resulting solution was stirred at 0° C. for 1 hr. The resulting mixture was applied to a 40 g C18 column and purified by Combi Flash (Biotage Isolera Prime), eluted with 5˜20% acetonitrile in water within 45 min to afford 2′-(1-hydroxyethyl)-5′-methoxy-N-(5-methoxy-1,3,4-thiadiazol-2-yl)-6-methyl-(4,4′-bipyridine)-3-carboxamide (150.0 mg, 32%) as a white solid. MS (ESI) calc'd for (C₁₈H₁₉N₅O₄S) (M+1)⁺, 402.1. found, 402.2.

### Step 5 (R)-2′-(1-hydroxyethyl)-5′-methoxy-N-(5-methoxy-1,3,4-thiadiazol-2-yl)-6-methyl-(4,4′-bipyridine)-3-carboxamide and (S)-2′-(1-hydroxyethyl)-5′-methoxy-N-(5-methoxy-1,3,4-thiadiazol-2-yl)-6-methyl-(4,4′-bipyridine)-3-carboxamide

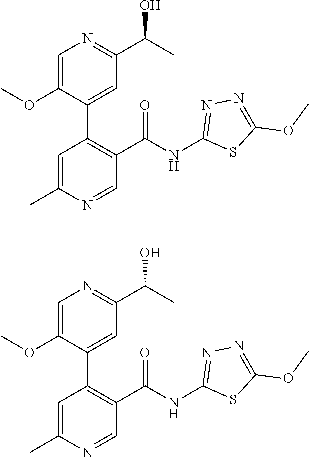

The racemic compound (150.0 mg) was separated by prep-chiral HPLC with the following condition: (Column: CHIRALPAK IE, 2*25 cm, 5 um; Mobile Phase A: MtBE—HPLC, Mobile Phase B: MeOH—HPLC; Flow rate: 16 mL/min; Gradient: 10% B to 10% B in 23 min; Wave Length: 220/254 nm; RT1 (min): 13.43; RT2 (min): 19.75; Sample Solvent: MeOH:DCM=1:1; Injection Volume: 1.1 mL; Number Of Runs: 3) to afford (R)-2′-(1-hydroxyethyl)-5′-methoxy-N-(5-methoxy-1,3,4-thiadiazol-2-yl)-6-methyl-(4,4′-bipyridine)-3-carboxamide (56.6 mg, 37%) as a white solid with shorter retention time on chiral-HPLC and (S)-2′-(1-hydroxyethyl)-5′-methoxy-N-(5-methoxy-1,3,4-thiadiazol-2-yl)-6-methyl-(4,4′-bipyridine)-3-carboxamide (58.0 mg, 38%) as a white solid with longer retention time on chiral-HPLC. The absolute stereochemistry was not determined and was arbitrarily assigned.

(R)-2′-(1-hydroxyethyl)-5′-methoxy-N-(5-methoxy-1,3,4-thiadiazol-2-yl)-6-methyl-(4,4′-bipyridine)-3-carboxamide: MS (ESI) calculated for (C₁₈H₁₉N₅O₄S) (M+1)⁺, 402.1. found, 402.2. ¹H NMR (400 MHz, DMSO-d₆) δ 12.89 (s, 1H), 8.76 (s, 1H), 8.23 (s, 1H), 7.45 (s, 1H), 7.36 (s, 1H), 5.35 (d, J=4.8 Hz, 1H), 4.81-4.71 (m, 1H), 4.08 (s, 3H), 3.62 (s, 3H), 2.60 (s, 3H), 1.40 (d, J=6.4 Hz, 3H).

(S)-2′-(1-hydroxyethyl)-5′-methoxy-N-(5-methoxy-1,3,4-thiadiazol-2-yl)-6-methyl-(4,4′-bipyridine)-3-carboxamide: MS (ESI) calculated for (C₁₈H₁₉N₅O₄S) (M+1)⁺, 402.1. found, 402.2. ¹H NMR (400 MHz, DMSO-d₆) δ 12.88 (s, 1H), 8.76 (s, 1H), 8.23 (s, 1H), 7.45 (s, 1H), 7.36 (s, 1H), 5.34 (d, J=4.8 Hz, 1H), 4.81-4.70 (m, 1H), 4.08 (s, 3H), 3.62 (s, 3H), 2.60 (s, 3H), 1.40 (d, J=6.4 Hz, 3H).

### Example 70

### 2′-(difluoromethyl)-5′-methoxy-N-(5-methoxy-1,3,4-thiadiazol-2-yl)-6-methyl-(4,4′-bipyridine)-3-carboxamide

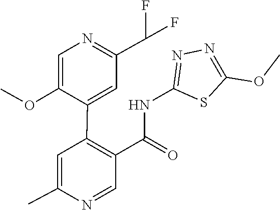

### Step 1 methyl 2′-chloro-5′-methoxy-6-methyl-(4,4′-bipyridine)-3-carboxylate

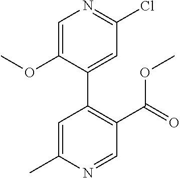

To a stirred solution of 2′-chloro-5′-methoxy-6-methyl-(4,4′-bipyridine)-3-carboxylic acid (3.0 g, 10.76 mmol) in dichloromethane (30 mL) were sequentially added methanol (3 mL) and (TriMethylsilyl) diazoMethane (2.5 g, 21.54 mmol) at 0° C. The resulting solution was stirred at room temperature for 16 hr under nitrogen. The organic solvent was removed under vacuum. The resulting residue was purified by Combi Flash (Biotage Isolera Prime) which applied to 80 g silica gel column and eluted with 0˜65% ethyl acetate in petroleum ether within 40 min to afford methyl 2′-chloro-5′-methoxy-6-methyl-(4,4′-bipyridine)-3-carboxylate (2.5 g, 78%) as a white solid. MS (ESI) calc'd for (C₁₄H₁₃ClN₂O₃) (M+1)⁺, 293.1. found 293.2.

### Step 2 methyl 5′-methoxy-6-methyl-2′-vinyl-(4,4′-bipyridine)-3-carboxylate

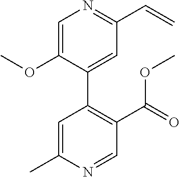

To a stirred solution of methyl 2′-chloro-5′-methoxy-6-methyl-(4,4′-bipyridine)-3-carboxylate (500.0 mg, 1.71 mmol) in 1,4-dioxane (4.5 mL) were sequentially added water (1.5 mL), trifluoro(vinyl)-14-borane, potassium salt (343.0 mg, 2.56 mmol), 1,1′-bis(diphenylphosphino)ferrocene-palladium(II)dichloride dichloromethane complex (139.0 mg, 0.17 mmol) and K₂CO₃ (708.0 mg, 5.12 mmol) at 23° C. The resulting solution was stirred at 80° C. for 2 hr under nitrogen. The suspension was filtered. The filtrate was collected and concentrated under vacuum. The resulting residue was dissolved in acetonitrile (4 mL) which was applied to a 40 g C18 column and purified by Combi Flash (Biotage Isolera Prime), eluted with 5˜35% acetonitrile in water within 30 min to afford methyl 5′-methoxy-6-methyl-2′-vinyl-(4,4′-bipyridine)-3-carboxylate (420.0 mg, 85%) as a white solid. MS (ESI) calc'd for (C₁₆H₁₆N₂O₃) (M+1)⁺, 285.1. found, 285.3.

### Step 3 methyl 2′-formyl-5′-methoxy-6-methyl-(4,4′-bipyridine)-3-carboxylate

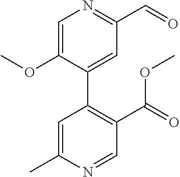 [SMILES: COC(=O)c1cnc(C)cc1-c1cc(C=O)ncc1OC]

To a stirred solution of methyl 5′-methoxy-6-methyl-2′-vinyl-(4,4′-bipyridine)-3-carboxylate (370.0 mg, 1.30 mmol) in tetrahydrofuran (10 mL) were sequentially added water (10 mL), sodium periodate (1.1 g, 5.21 mmol) and osmium tetroxide (0.04 mL, 0.13 mmol) at 20° C. The resulting solution was stirred at 20° C. for 1 hr under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in dichloromethane (3 mL) and purified by Combi Flash (Biotage Isolera Prime) which applied to a 40 g silica gel column that was eluted with 0˜71% ethyl acetate in petroleum ether within 30 min to afford methyl 2′-formyl-5′-methoxy-6-methyl-(4,4′-bipyridine)-3-carboxylate (255.0 mg, 67%) as a white solid. MS (ESI) calc'd for (C₁₅H₁₄N₂O₄) (M+1)⁺, 287.1. found 287.2.

### Step 4 methyl 2′-(difluoromethyl)-5′-methoxy-6-methyl-(4,4′-bipyridine)-3-carboxylate

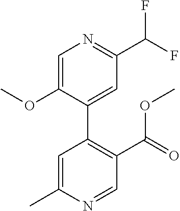

To a stirred solution of methyl 2′-formyl-5′-methoxy-6-methyl-(4,4′-bipyridine)-3-carboxylate (230.0 mg, 0.80 mmol) in dichloromethane (2 mL) was added DAST (0.2 mL, 1.61 mmol) at 0° C. The resulting solution was stirred at 20° C. for 48 hr under nitrogen. The reaction mixture was quenched by the addition of water and extracted with ethyl acetate. The combined organic layers were washed with brine, dried over sodium sulfate and concentrated under vacuum. The resulting residue was dissolved in acetonitrile (3 mL) which was applied to a 40 g C18 column and purified by Combi Flash (Biotage Isolera Prime), eluted with 5˜40% acetonitrile in water within 30 min to afford methyl 2′-(difluoromethyl)-5′-methoxy-6-methyl-(4,4′-bipyridine)-3-carboxylate (160.0 mg, 63%) as a brown oil. MS (ESI) calc'd for (C₁₅H₁₄F₂N₂O₃) (M+1)⁺, 309.1. found 309.2.

### Step 5 2′-(difluoromethyl)-5′-methoxy-6-methyl-(4,4′-bipyridine)-3-carboxylic Acid

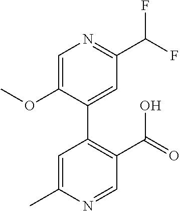 [SMILES: COc1cnc(C(F)F)cc1-c1cc(C)ncc1C(=O)O]

To a stirred solution of methyl 2′-(difluoromethyl)-5′-methoxy-6-methyl-(4,4′-bipyridine)-3-carboxy late (160.0 mg, 0.52 mmol) in tetrahydrofuran (1.5 mL) were sequentially added water (0.5 mL) and lithium hydroxide (49.7 mg, 2.08 mmol) at 23° C. The resulting solution was stirred at 23° C. for 2 hr under nitrogen. The organic solvent was removed under vacuum. The aqueous layer was acidified with citric acid solution to pH 5˜6 and extracted with ethyl acetate. The combined organic layers were washed with brine, dried over anhydrous sodium sulfate, filtered, and concentrated under vacuum to afford 2′-(difluoromethyl)-5′-methoxy-6-methyl-(4,4′-bipyridine)-3-carboxylic acid (150.0 mg, 93%) as a yellow solid. MS (ESI) calc'd for (C₁₄H₁₂F₂N₂O₃) (M+1)⁺, 295.1. found 295.2.

### Step 6 2′-(difluoromethyl)-5′-methoxy-N-(5-methoxy-1,3,4-thiadiazol-2-yl)-6-methyl-(4,4′-bipyridine)-3-carboxamide

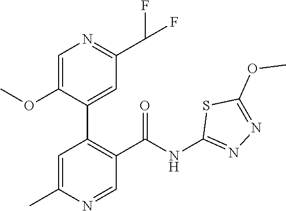

To a stirred solution of 2′-(difluoromethyl)-5′-methoxy-6-methyl-(4,4′-bipyridine)-3-carboxylic acid (200.0 mg, 0.68 mmol) and 5-methoxy-1,3,4-thiadiazol-2-amine (89.0 mg, 0.68 mmol) in acetonitrile (2 mL) was added 1-methylimidazole (0.2 mL, 2.72 mmol). Then a solution of TCFH (191.0 mg, 0.68 mmol) in acetonitrile (2 mL) was added to the above mixture at 23° C. The resulting solution was stirred at 23° C. for 2 hr under nitrogen. The reaction mixture (2 mL) was purified by prep-HPLC with the following condition: (Column: XBridge Prep Phenyl OBD Column, 19*250 mm, 5 um; Mobile Phase A: Water (10 mmol/L NH₄HCO₃), Mobile Phase B: ACN; Flow rate: 25 mL/min; Gradient: 27% B to 27% B in 8 min, 27% B to 95% B in 9.2 min, 95% B to 95% B in 10.5 min, 95% B to 5% B in 11 min, 5% B; Wave Length: 254 nm; RT1 (min): 7; Injection Volume: 05 mL; Number Of Runs: 4) to afford 2′-(difluoromethyl)-5′-methoxy-N-(5-methoxy-1,3,4-thiadiazol-2-yl)-6-methyl-(4,4′-bipyridine)-3-carboxamide (78.5 mg, 27%) as a white solid. MS (ESI) calc'd for (C₁₇H₁₅F₂N₅O₃S) (M+1)⁺, 408.1. found 408.1. ¹H NMR (400 MHz, DMSO-d₆) δ 8.88 (s, 1H), 8.44 (s, 1H), 7.61 (s, 1H), 7.32 (s, 1H), 7.15-6.82 (m, 1H), 4.02 (s, 3H), 3.70 (s, 3H), 2.57 (s, 3H).

### Example 71

### N-(5-methoxy-1,3,4-thiadiazol-2-yl)-4-(5-methoxy-1H-benzo(d)imidazol-6-yl)-6-methylnicotinamide

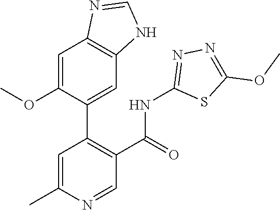

### Step 1 methyl 4-(5-methoxy-1H-benzo(d)imidazol-6-yl)-6-methylnicotinate

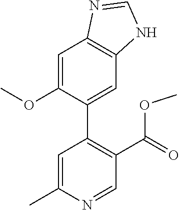

To a stirred solution of 6-bromo-5-methoxy-1H-benzo(d)imidazole (500.0 mg, 2.20 mmol) in 1,4-Dioxane (5 mL) was added methyl 6-methyl-4-(4,4,5,5-tetramethyl-1,3,2-dioxaborolan-2-yl) nicotinate (1.2 g, 4.40 mmol) at 23ºC. To the above solution were added K₂CO₃ (913.0 mg, 6.61 mmol) in Water (1 mL) and Pd (dtbpf)Cl₂ (143.2 mg, 0.22 mmol) at 23° C. The resulting mixture was then stirred at 80° C. for 2 hr under nitrogen. The reaction mixture was quenched with water and extracted with ethyl acetate. The combined organic layers were washed with brine, dried over anhydrous sodium sulfate, filtered, and concentrated under vacuum. The resulting residue was dissolved in DCM (3 mL) and purified by Combi Flash (Biotage Isolera Prime) which applied to a 40 g silica gel column that was eluted with 0˜20% methanol in dichloromethane within 35 min to afford methyl 4-(5-methoxy-1H-benzo(d)imidazol-6-yl)-6-methylnicotinate (142.0 mg, 20%) as a yellow oil. MS (ESI) calc'd for (C₁₆H₁₅N₃O₃) (M+1)⁺, 298.1. found 298.1.

### Step 2: 4-(5-methoxy-1H-benzo(d)imidazol-6-yl)-6-methylnicotinic Acid

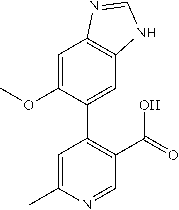 [SMILES: COc1cc2nc[nH]c2cc1-c1cc(C)ncc1C(=O)O]

To a stirred solution of methyl 4-(5-methoxy-1H-benzo(d)imidazol-6-yl)-6-methylnicotinate (140.0 mg, 0.47 mmol) in THF (1.5 mL) and Water (1.5 mL) was added LiOH (22.6 mg, 0.94 mmol) at 23° C. The resulting solution was stirred at 50° C. for 2 hr. The reaction mixture was acidified with Citric acid to pH ˜5 and extracted with ethyl acetate. The combined organic layers were washed with brine, dried over anhydrous sodium sulfate, filtered, and concentrated under vacuum to afford 4-(5-methoxy-1H-benzo(d)imidazol-6-yl)-6-methylnicotinic acid (60.0 mg, crude) as a white solid. MS (ESI) calc'd for (C₁₅H₁₃N₃O₃) (M+1)⁺, 284.1. found, 284.1.

### Step 3 N-(5-methoxy-1,3,4-thiadiazol-2-yl)-4-(5-methoxy-1H-benzo(d)imidazol-6-yl)-6-methylnicotinamide

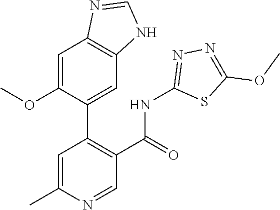

To a stirred solution of 4-(5-methoxy-1H-benzo(d)imidazol-6-yl)-6-methylnicotinic acid (50.0 mg, 0.17 mmol), 5-methoxy-1,3,4-thiadiazol-2-amine (23.2 mg, 0.17 mmol) and 1-methylimidazole (72.5 mg, 0.88 mmol) in Acetonitrile (1 mL). To the above was added a solution of TCFH (49.6 mg, 0.17 mmol) in Acetonitrile (1 mL). The resulting mixture was stirred at 23° C. for 2 hr. The mixture was dissolved in DMF (2 mL) and was purified by prep-HPLC with the following condition: (Column: X Bridge Prep Phenyl OBD Column, 19*250 mm, 5 um; Mobile Phase A: Water (10 mmol/L NH₄HCO₃), Mobile Phase B: MeOH—HPLC; Flow rate: 25 mL/min; Gradient: 48% B to 48% B in 10 min, 48% B to 95% B in 11.2 min, 95% B to 95% B in 12.5 min, 95% B to 5% B in 13 min, 5% B; Wave Length: 254 nm; RT1 (min): 7) to afford N-(5-methoxy-1,3,4-thiadiazol-2-yl)-4-(5-methoxy-1H-benzo(d)imidazol-6-yl)-6-methylnicotinamide (14.4 mg, 20%) as a white solid. MS (ESI) calc'd for (C₁₈H₁₆N₆O₃S) (M+1)⁺, 397.1. found 397.2. ¹H NMR (400 MHz, DMSO-d₆) δ 12.48 (br, 2H), 8.66 (s, 1H), 8.16 (s, 1H), 7.53 (s, 1H), 7.33 (s, 1H), 7.06 (s, 1H), 4.04 (s, 3H), 3.53 (s, 3H), 2.57 (s, 3H).

### Example 72

### 2′-chloro-5′-(hydroxymethyl)-N-(5-methoxy-1,3,4-thiadiazol-2-yl)-6-methyl-(4,4′-bipyridine)-3-carboxamide

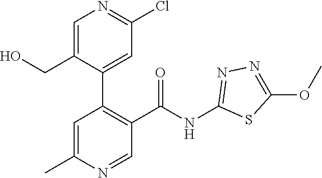 [SMILES: COc1nnc(NC(=O)c2cnc(C)cc2-c2cc(Cl)ncc2CO)s1]

### Step 1 methyl 5′-bromo-2′-chloro-6-methyl-(4,4′-bipyridine)-3-carboxylate

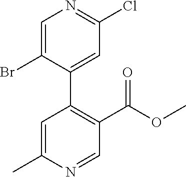

To a solution of 5-bromo-2-chloro-4-iodopyridine (1.0 g, 3.24 mmol) and methyl 6-methyl-4-(4,4,5,5-tetramethyl-1,3,2-dioxaborolan-2-yl)nicotinate (2.7 g, 9.71 mmol) in 1,4-Dioxane (5 mL) and Water (1 mL) were added K₃PO₄ (0.9 g, 6.47 mmol) and Pd(PPh₃)₂Cl₂ (0.2 g, 0.32 mmol) at 20° C. under nitrogen. The resulting solution was stirred at 80° C. for 2 hr under nitrogen. The reaction mixture was extracted with ethyl acetate. The combined organic layers were washed with brine, dried over anhydrous sodium sulfate, filtered, and concentrated under vacuum. The resulting residue was dissolved in DCM (2 mL) and purified by Combi Flash (Biotage Isolera Prime) which applied to a 40 g silica gel column that was eluted with 0˜50% ethyl acetate in petroleum ether within 35 min to afford methyl 5′-bromo-2′-chloro-6-methyl-(4,4′-bipyridine)-3-carboxylate (770.0 mg, 51%) as a yellow solid. MS (ESI) calc'd for (C₁₃H₁₀BrClN₂O₂) (M+1)⁺, 341.0. found 341.0.

### Step 2: methyl 2′-chloro-6-methyl-5′-vinyl-(4,4′-bipyridine)-3-carboxylate

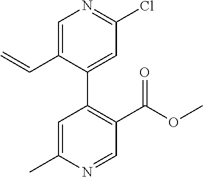 [SMILES: C=Cc1cnc(Cl)cc1-c1cc(C)ncc1C(=O)OC]

To a solution of methyl 5′-bromo-2′-chloro-6-methyl-(4,4′-bipyridine)-3-carboxylate (770.0 mg, 2.25 mmol) and 4,4,5,5-tetramethyl-2-vinyl-1,3,2-dioxaborolane (347.0 mg, 2.25 mmol) in Toluene (4 mL) and Water (1 mL) were added potassium phosphate (393.0 mg, 2.25 mmol) and Pd(dtbpf)Cl₂ (1039.0 mg, 2.25 mmol) at 20° C. under nitrogen. The resulting solution was stirred at 100° C. for 2 hr under nitrogen. The reaction mixture was extracted with ethyl acetate. The combined organic layers were washed with brine, dried over anhydrous sodium sulfate, filtered, and concentrated under vacuum. The resulting residue was dissolved in DMF (2 mL) which was applied to a 40 g C18 column and purified by Combi Flash (Biotage Isolera Prime), eluted with 5˜75% acetonitrile in water within 25 min to afford methyl 2′-chloro-6-methyl-5′-vinyl-(4,4′-bipyridine)-3-carboxylate (240.0 mg, 33%) as a yellow oil. MS (ESI) calc'd for (C₁₅H₁₃ClN₂O₂) (M+1)⁺, 289.1. found 289.1.

### Step 3 2′-chloro-6-methyl-5′-vinyl-(4,4′-bipyridine)-3-carboxylic Acid

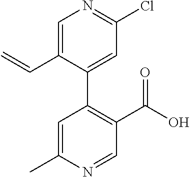 [SMILES: C=Cc1cnc(Cl)cc1-c1cc(C)ncc1C(=O)O]

To a stirred solution of methyl 2′-chloro-6-methyl-5′-vinyl-(4,4′-bipyridine)-3-carboxylate (30.0 mg, 0.11 mmol) in THF (1 mL) was added a solution of lithium hydroxide (3.0 mg, 0.11 mmol) in Water (0.2 mL) at 0° C. The resulting solution was stirred at 20° C. for 1 hr. The organic solvent was removed under vacuum. The aqueous layer was acidified with citric acid to pH ˜5 and extracted with ethyl acetate. The combined organic layers were washed with brine, dried over anhydrous sodium sulfate, filtered, and concentrated under vacuum to afford 2′-chloro-6-methyl-5′-vinyl-(4,4′-bipyridine)-3-carboxylic acid (40.0 mg, crude) as a yellow solid. MS (ESI) calc'd for (C₁₄H₁₁ClN₂O₂) (M+1)⁺, 275.1. found 275.1.

### Step 4 2′-chloro-N-(5-methoxy-1,3,4-thiadiazol-2-yl)-6-methyl-5′-vinyl-(4,4′-bipyridine)-3-carboxamide

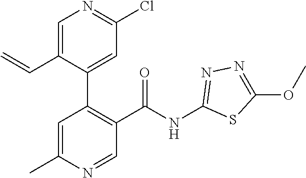 [SMILES: C=Cc1cnc(Cl)cc1-c1cc(C)ncc1C(=O)Nc1nnc(OC)s1]

To a solution of 2′-chloro-6-methyl-5′-vinyl-(4,4′-bipyridine)-3-carboxylic acid (40.0 mg, 0.14 mmol) in Acetonitrile (0.5 mL) was added 5-methoxy-1,3,4-thiadiazol-2-amine (19.1 mg, 0.14 mmol) and 1-methylimidazole (59.6 mg, 0.73 mmol) at 20° C. under nitrogen. To the above solution was added TCFH (40.8 mg, 0.14 mmol) in Acetonitrile (0.5 mL) at 20° C. under nitrogen. The resulting mixture was then stirred at 20° C. for 1 hr. The suspension was filtered. The filter cake was collected and dried under vacuum to afford 2′-chloro-N-(5-methoxy-1,3,4-thiadiazol-2-yl)-6-methyl-5′-vinyl-(4,4′-bipyridine)-3-carboxamide (18.0 mg, 32%) as a white solid. MS (ESI) calc'd for (C₁₇H₁₄ClN₅O₂S) (M+1)⁺, 388.1. found 388.1.

### Step 5 2′-chloro-5′-formyl-N-(5-methoxy-1,3,4-thiadiazol-2-yl)-6-methyl-(4,4′-bipyridine)-3-carboxamide

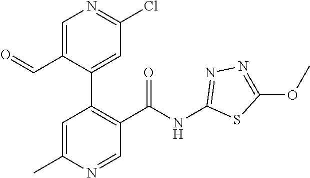 [SMILES: COc1nnc(NC(=O)c2cnc(C)cc2-c2cc(Cl)ncc2C=O)s1]

To a solution of 2′-chloro-N-(5-methoxy-1,3,4-thiadiazol-2-yl)-6-methyl-5′-vinyl-(4,4′-bipyridine)-3-carboxamide (18.0 mg, 0.04 mmol) in Tetrahydrofuran (THF) (1 mL) and Water (0.3 mL) were added osmium tetroxide (11.2 mg, 0.04 mmol) and sodium periodate (9.9 mg, 0.04 mmol) at 20° C. under nitrogen. The resulting solution was stirred at 20° C. for 2 hr. The reaction mixture was extracted with ethyl acetate. The combined organic layers were washed with brine, dried over anhydrous sodium sulfate, filtered, and concentrated under vacuum to afford 2′-chloro-5′-formyl-N-(5-methoxy-1,3,4-thiadiazol-2-yl)-6-methyl-(4,4′-bipyridine)-3-carboxamide (34.0 mg, crude) as a yellow solid. MS (ESI) calc'd for (C₁₆H₁₂ClN₅O₅S) (M+1)⁺, 390.0. found 390.0.

### Step 6 2′-chloro-5′-(hydroxymethyl)-N-(5-methoxy-1,3,4-thiadiazol-2-yl)-6-methyl-(4,4′-bipyridine)-3-carboxamide

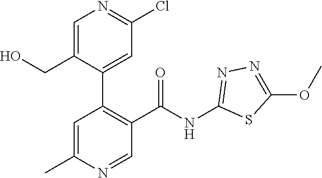 [SMILES: COc1nnc(NC(=O)c2cnc(C)cc2-c2cc(Cl)ncc2CO)s1]

To a stirred solution of 2′-chloro-5′-formyl-N-(5-methoxy-1,3,4-thiadiazol-2-yl)-6-methyl-(4,4′-bipyridine)-3-carboxamide (34.0 mg, 0.08 mmol) in Methanol (1 mL) was added sodium borohydride (6.6 mg, 0.17 mmol) at 0° C. The resulting solution was stirred at 0° C. for 1 hr. The resulting mixture was applied to a 20 g C18 column and purified by Combi Flash (Biotage Isolera Prime), eluted with 5˜55% acetonitrile in water within 35 min to afford 2′-chloro-5′-(hydroxymethyl)-N-(5-methoxy-1,3,4-thiadiazol-2-yl)-6-methyl-(4,4′-bipyridine)-3-carboxamide (8.3 mg, 23%) as a white solid. MS (ESI) calc'd for (C₁₆H₁₄ClN₅O₃S) (M+1)⁺, 392.1. found 392.1. ¹H NMR (400 MHz, DMSO-d₆) δ 9.05 (s, 1H), 8.40 (s, 1H), 7.15 (s, 1H), 7.00 (s, 1H), 5.53 (s, 1H), 4.21 (s, 2H), 3.89 (s, 3H), 2.50 (s, 3H).

### Example 73

### 2′-amino-5′-methoxy-N-(5-methoxy-1,3,4-thiadiazol-2-yl)-6-methyl-(4,4′-bipyridine)-3-carboxamide

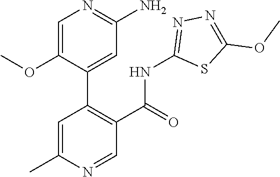

### Step 1 methyl 2′-chloro-5′-methoxy-6-methyl-(4,4′-bipyridine)-3-carboxylate

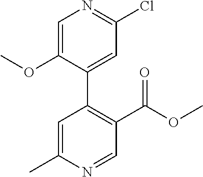

To a stirred solution of 2′-chloro-5′-methoxy-6-methyl-(4,4′-bipyridine)-3-carboxylic acid (2.0 g, 7.18 mmol) in N,N-Dimethylformamide (20 mL) under nitrogen atmosphere were added K₂CO₃ (1.9 g, 14.35 mmol) and MeI (1.5 g, 10.76 mmol) at 25° C. The reaction mixture was stirred at 25° C. for 16 h under nitrogen atmosphere. The reaction mixture was quenched by the addition of water and extracted with ethyl acetate. The combined organic layers were washed with brine, dried over anhydrous sodium sulfate, filtered, and concentrated under vacuum. The residue was dissolved with DMF (8 mL), purified by Combi Flash (Biotage Isolera Prime), eluted with 5˜48% acetonitrile in water within 40 min to afford methyl 2′-chloro-5′-methoxy-6-methyl-(4,4′-bipyridine)-3-carboxylate (1.4 g, 67%) as a white solid. MS (ESI) calc'd for (C₁₄H₁₃ClN₂O₃) (M+1)⁺, 293.0. found 293.1.

### Step 2 methyl 2′-((diphenylmethylene)amino)-5′-methoxy-6-methyl-(4,4′-bipyridine)-3-carboxylate

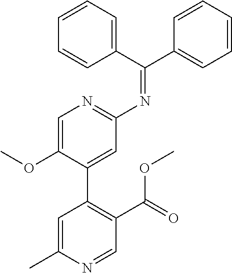 [SMILES: COC(=O)c1cnc(C)cc1-c1cc(N=C(c2ccccc2)c2ccccc2)ncc1OC]

To a stirred solution of methyl 2′-chloro-5′-methoxy-6-methyl-(4,4′-bipyridine)-3-carboxylate (500.0 mg, 1.70 mmol) in 1,4-Dioxane (6 mL) were added diphenylmethanimine (464.0 mg, 2.56 mmol), Cs₂CO₃ (1670.0 mg, 5.12 mmol), Pd(OAc)₂ (38.3 mg, 0.17 mmol) and BINAP (213.0 mg, 0.34 mmol). The mixture was stirred at 80° C. under nitrogen atmosphere for 16 h. The mixture was filtered. The filtrate was collected and concentrated under vacuum. The resulting residue was dissolved in DMF (5 mL), applied to a 80 g C18 column and purified by Combi Flash (Biotage Isolera Prime), eluted with 5˜51% acetonitrile in water within 30 min to afford methyl 2′-((diphenylmethylene)amino)-5′-methoxy-6-methyl-(4,4′-bipyridine)-3-carboxylate (600.0 mg, 79%) as a yellow solid. MS (ESI) calc'd for (C₂₇H₂₃N₃O₃) (M+1)⁺, 438.1. found 438.1.

### Step 3: 2′-((diphenylmethylene)amino)-5′-methoxy-6-methyl-(4,4′-bipyridine)-3-carboxylic Acid

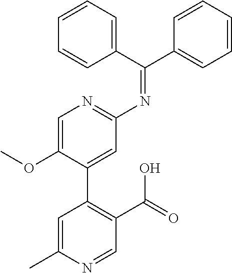 [SMILES: COc1cnc(N=C(c2ccccc2)c2ccccc2)cc1-c1cc(C)ncc1C(=O)O]

To a stirred solution of methyl 2′-((diphenylmethylene)amino)-5′-methoxy-6-methyl-(4,4′-bipyridine)-3-carboxylate (560.0 mg, 1.28 mmol) in Methanol (3 mL) and Water (3 mL) was added NaOH (205.0 mg, 5.12 mmol). The resulting solution was stirred at 60° C. for 2 h. The organic solvent was removed under vacuum. The aqueous layer was acidified with citric acid to pH ˜6 and extracted with ethyl acetate. The combined organic layers were washed with brine, dried over anhydrous sodium sulfate, filtered, and concentrated under vacuum to afford 2′-((diphenylmethylene)amino)-5′-methoxy-6-methyl-(4,4′-bipyridine)-3-carboxylic acid (500.0 mg, crude) as a yellow solid. MS (ESI) calc'd for (C₂₆H₂₁N₃O₃) (M+1)⁺, 424.1. found 424.2.

### Step 4 2′-((diphenylmethylene)amino)-5′-methoxy-N-(5-methoxy-1,3,4-thiadiazol-2-yl)-6-methyl-(4,4′-bipyridine)-3-carboxamide

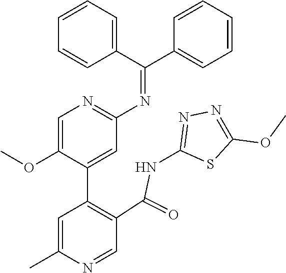

To a stirred solution of 2′-((diphenylmethylene)amino)-5′-methoxy-6-methyl-(4,4′-bipyridine)-3-carboxylic acid (480.0 mg, 1.13 mmol) in Acetonitrile (5 mL) were sequentially added 5-methoxy-1,3,4-thiadiazol-2-amine (149.0 mg, 1.13 mmol) and 1-methyl-1H-imidazole (465.0 mg, 5.67 mmol) at 25° C. under nitrogen. To the above solution was added a solution of TCFH (318.0 mg, 1.133 mmol) in Acetonitrile (1 mL) at 25° C. under nitrogen. The resulting mixture was then stirred at 25° C. for 2 h. The resulting solution (6 mL) was applied to a 80 g C18 column and purified by Combi Flash (Biotage Isolera Prime), eluted with 5˜45% acetonitrile in water within 45 min to afford 2′-((diphenylmethylene)amino)-5′-methoxy-N-(5-methoxy-1,3,4-thiadiazol-2-yl)-6-methyl-(4,4′-bipyridine)-3-carboxamide (320.0 mg, 50%) as a yellow solid. MS (ESI) calc'd for (C₂₉H₂₄N₆O₃S) (M+1)⁺, 537.1. found 537.3.

### Step 5 2′-amino-5′-methoxy-N-(5-methoxy-1,3,4-thiadiazol-2-yl)-6-methyl-(4,4′-bipyridine)-3-carboxamide

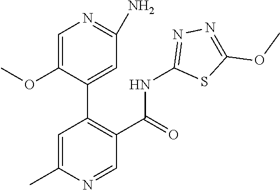

To a stirred solution of 2′-((diphenylmethylene)amino)-5′-methoxy-N-(5-methoxy-1,3,4-thiadiazol-2-yl)-6-methyl-(4,4′-bipyridine)-3-carboxamide (300.0 mg, 0.55 mmol) in Tetrahydrofuran (3 mL) was added HCl (0.6 mL, 1.20 mmol, 2 N) at 25° C. The resulting solution was stirred at 25° C. for 2 h and concentrated under vacuum. The resulting residue was dissolved in ACN (2 mL) and purified by purified by prep-HPLC with the following condition: (Column: Xselect CSH C18 OBD Column 30*150 mm 5 μm; Mobile Phase A: Water (0.1% FA), Mobile Phase B: ACN; Flow rate: 60 mL/min; Gradient: 6% B to 13% B in 8 min, 13% B; Wave Length: 220 nm; RT1 (min): 5.43; Injection Volume: 0.6 mL; Number Of Runs: 4) to afford 2′-amino-5′-methoxy-N-(5-methoxy-1,3,4-thiadiazol-2-yl)-6-methyl-(4,4′-bipyridine)-3-carboxamide (64.3 mg, 30%) as a white solid. MS (ESI) calc'd for (C₁₆H₁₆N₆O₃S) (M+1)⁺, 373.1. found 373.1. ¹H NMR (400 MHz, DMSO-d₆+D₂O) δ 8.67 (s, 1H), 7.58 (s, 1H), 7.29 (s, 1H), 6.50 (s, 1H), 4.04 (s, 3H), 3.42 (s, 3H), 2.56 (s, 3H).

### Example 74

### 2′-chloro-5′-methoxy-6-methyl-N-(5-((2-methyltetrahydrofuran-3-yl)methoxy)-1,3,4-thiadiazol-2-yl)-(4,4′-bipyridine)-3-carboxamide

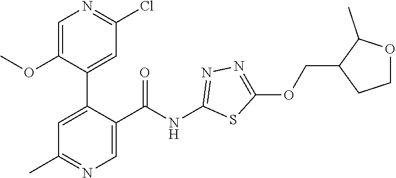 [SMILES: COc1cnc(Cl)cc1-c1cc(C)ncc1C(=O)Nc1nnc(OCC2CCOC2C)s1]

### Step 1 S-methyl O-((2-methyltetrahydrofuran-3-yl)methyl) carbonodithioate

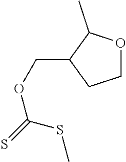

To a mixture of NaH (41.3 mg, 1.03 mmol, 60%) in THF (2 mL) was added (2-methyltetrahydrofuran-3-yl) methanol (100.0 mg, 0.86 mmol) in portions at 0° C. and stirred at 0° C. for 1 hr. Then CS₂ (98.1 mg, 1.29 mmol) was added to the above mixture and stirred at 0° C. for 10 min, and then MeI (183.1 mg, 1.29 mmol) was added to the above mixture at 5° C. The resulting mixture was stirred at room temperature for 1 hour. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (2 mL) and purified by Combi Flash (Biotage Isolera Prime) which applied to a 20 g silica gel column that was eluted with 0˜20% ethyl acetate in petroleum ether within 20 min. to afford S-methyl O-((2-methyltetrahydrofuran-3-yl)methyl) carbonodithioate (140.0 mg, 76%) as a colorless oil. MS (ESI) calc'd for (C₈H₁₄O₂S₂) (M+1)⁺, 206.0.

### Step 2 O-((2-methyltetrahydrofuran-3-yl)methyl) hydrazinecarbothioate

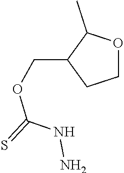

To a stirred solution of S-methyl O-((2-methyltetrahydrofuran-3-yl)methyl) carbonodithioate (140.0 mg, 0.68 mmol) in Methanol (1 mL) was added hydrazine (24.0 mg, 0.75 mmol, 80%) at 23° C. The resulting solution was stirred at 23° C. for 1 hr. The organic solvent was removed under vacuum to afford O-((2-methyltetrahydrofuran-3-yl)methyl) hydrazinecarbothioate (130.0 mg, crude) as a colorless oil. MS (ESI) calc'd for (C₇H₁₄N₂O₂S) (M+1)⁺, 191.1.

### Step 3 5-((2-methyltetrahydrofuran-3-yl)methoxy)-1,3,4-thiadiazol-2-amine

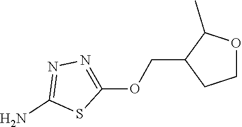

To a stirred solution of O-((2-methyltetrahydrofuran-3-yl)methyl) hydrazinecarbothioate (130.0 mg, 0.68 mmol) in Methanol (1 mL) were added TEA (138.4 mg, 1.37 mmol) and cyanic bromide (80.0 mg, 0.75 mmol) at 23° C. The resulting solution was stirred at 23° C. for 1 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (2 mL) and purified by Combi Flash (Biotage Isolera Prime) which applied to a 20 g silica gel column that was eluted with 0˜10% methanol in dichloromethane within 25 min to afford 5-((2-methyltetrahydrofuran-3-yl)methoxy)-1,3,4-thiadiazol-2-amine (70.0 mg, 47%) as a yellow solid. MS (ESI) calc'd for (C₈H₁₃N₃O₂S) (M+1)⁺, 216.1. found 216.1.

### Step 4 2′-chloro-5′-methoxy-6-methyl-N-(5-((2-methyltetrahydrofuran-3-yl)methoxy)-1,3,4-thiadiazol-2-yl)-(4,4′-bipyridine)-3-carboxamide

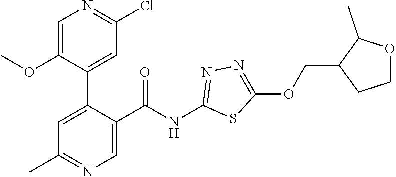 [SMILES: COc1cnc(Cl)cc1-c1cc(C)ncc1C(=O)Nc1nnc(OCC2CCOC2C)s1]

To a stirred solution of 5-((2-methyltetrahydrofuran-3-yl) methoxy)-1,3,4-thiadiazol-2-amine (60.0 mg, 0.28 mmol), Intermediate G (78.0 mg, 0.28 mmol) and 1-methylimidazole (114 mg, 1.39 mmol) in Acetonitrile (1.5 mL) and was added a solution of TCFH (78.0 mg, 0.28 mmol) in Acetonitrile (1.5 mL). The resulting mixture was stirred at 23° C. for 2 hr. The resulting mixture was applied to a 20 g C18 column and purified by Combi Flash (Biotage Isolera Prime), eluted with 5˜50% acetonitrile in water within 25 min to afford 2′-chloro-5′-methoxy-6-methyl-N-(5-((2-methyltetrahydrofuran-3-yl)methoxy)-1,3,4-thiadiazol-2-yl)-(4,4′-bipyridine)-3-carboxamide (83.2 mg, 62%) as a white solid. MS (ESI) calc'd for (C₂₁H₂₂ClN₅O₄S) (M+1)⁺, 476.1. found 476.2. ¹H NMR (400 MHz, DMSO-d₆) δ 12.90 (s, 1H), 8.81 (s, 1H), 8.18 (s, 1H), 7.54 (s, 1H), 7.43 (s, 1H), 4.49-4.41 (m, 1H), 4.40-4.26 (m, 1H), 4.03-3.96 (m, 1H), 3.90-3.79 (m, 1H), 3.72-3.54 (m, 4H), 2.65-2.52 (m, 4H), 2.15-1.97 (m, 1H), 1.80-1.74 (m, 1H), 1.20 (d, J=6.4 Hz, 1H), 1.11 (d, J=6.4 Hz, 2H).

### Example 75

### 2′-chloro-N-(5-methoxy-1,3,4-thiadiazol-2-yl)-6-methyl-(4,4′-bipyridine)-3-carboxamide

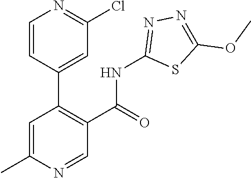

### Step 1 methyl 2′-chloro-6-methyl-(4,4′-bipyridine)-3-carboxylate

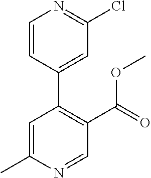 [SMILES: COC(=O)c1cnc(C)cc1-c1ccnc(Cl)c1]

To a stirred solution of 4-bromo-2-chloropyridine (100.0 mg, 0.52 mmol) in 1,4-Dioxane (5 mL) and Water (1 mL) were sequentially added methyl 6-methyl-4-(4,4,5,5-tetramethyl-1,3,2-dioxaborolan-2-yl) nicotinate (288.0 mg, 1.04 mmol), K₂CO₃ (215.0 mg, 1.56 mmol) and Pd(dppf)Cl₂ (76.0 mg, 0.10 mmol) at 20° C. The resulting solution was stirred at 100° C. under nitrogen atmosphere for 2 hr. The mixture was filtered. The filtrate was concentrated under vacuum. The resulting residue was dissolved in DMF (4 mL) which was applied to a 40 g C18 column and purified by Combi Flash (Biotage Isolera Prime), eluted with 5˜50% acetonitrile in water within 30 min to afford methyl 2′-chloro-6-methyl-(4,4′-bipyridine)-3-carboxylate (120.0 mg, 83%) as a gray solid. MS (ESI) calculated for (C₁₃H₁₁ClN₂O₂) (M+1)⁺, 263.0. found, 263.1.

### Step 2 2′-chloro-6-methyl-(4,4′-bipyridine)-3-carboxylic Acid

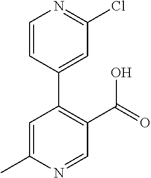 [SMILES: Cc1cc(-c2ccnc(Cl)c2)c(C(=O)O)cn1]

To a solution of methyl 2′-chloro-6-methyl-(4,4′-bipyridine)-3-carboxylate (340.0 mg, 1.29 mmol) in Tetrahydrofuran (2 mL) and Water (0.7 mL) was added lithium hydroxide (93.0 mg, 3.88 mmol) at 20° C. The resulting solution was stirred at 40° C. for 2 hr. The aqueous layer was acidified with citric acid to pH ˜4 and extracted with ethyl acetate. The combined organic layers were washed with brine, dried over anhydrous sodium sulfate, filtered, and concentrated under vacuum to afford 2′-chloro-6-methyl-(4,4′-bipyridine)-3-carboxylic acid (150.0 mg, crude) as a brown solid. MS (ESI) calculated for (C₁₂H₉ClN₂O₂) (M+1)⁺, 249.0. found, 249.0.

### Step 3 2′-chloro-N-(5-methoxy-1,3,4-thiadiazol-2-yl)-6-methyl-(4,4′-bipyridine)-3-carboxamide

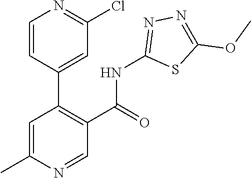

To a mixture of 5-methoxy-1,3,4-thiadiazol-2-amine (82.0 mg, 0.63 mmol) in acetonitrile (5 mL) were added 2′-chloro-6-methyl-(4,4′-bipyridine)-3-carboxylic acid (130.0 mg, 0.52 mmol) and NMI (215.0 mg, 2.61 mmol). To the above was added a solution of TCFH (161.0 mg, 0.58 mmol) in acetonitrile (1 mL) under nitrogen. The resulting solution was stirred at 20° C. for 2 hr. The organic solvent was removed under vacuum. The resulting residue was dissolved in DMF (3 mL) which was applied to a 40 g C18 column and purified by Combi Flash (Biotage Isolera Prime), eluted with 5%˜40% acetonitrile in water within 40 min to afford 2′-chloro-N-(5-methoxy-1,3,4-thiadiazol-2-yl)-6-methyl-(4,4′-bipyridine)-3-carboxamide (100.9 mg, 53%) as a white solid. MS (ESI) calculated for (C₁₅H₁₂ClN₅O₂S) (M+1)⁺, 362.0. found, 362.1. ¹H NMR (400 MHz, DMSO-d₆), δ 13.03 (br, 1H), 8.85 (s, 1H), 8.46 (d, J=5.2 Hz, 1H), 7.58 (d, J=1.6 Hz, 1H), 7.51 (s, 1H), 7.36 (dd, J=5.2, 1.6 Hz, 1H), 4.08 (s, 3H), 2.61 (s, 3H).

### Example 76 and 77

### 2′-chloro-5′-methoxy-6-methyl-N-(5-(((2r,3r)-2-methyltetrahydrofuran-3-yl)oxy)-1,3,4-thiadiazol-2-yl)-(4,4′-bipyridine)-3-carboxamide and 2′-chloro-5′-methoxy-6-methyl-N-(5-(((2r,3s)-2-methyltetrahydrofuran-3-yl)oxy)-1,3,4-thiadiazol-2-yl)-(4,4′-bipyridine)-3-carboxamide

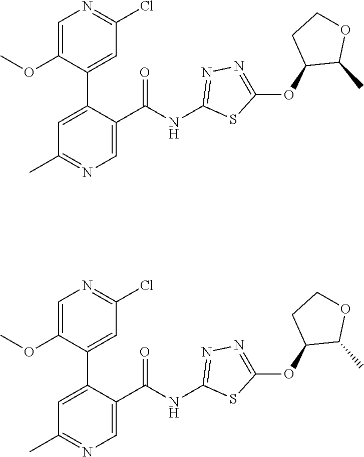

### Step 1 S-methyl O-(2-methyltetrahydrofuran-3-yl) carbonodithioate

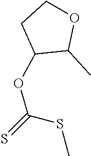

To a solution of 2-methyltetrahydrofuran-3-ol (300.0 mg, 2.94 mmol) in THF (10 mL) was added NaH (141.0 mg, 3.52 mmol, 60%) in portions at 0° C. and stirred at 0° C. for 30 min. Then CS₂ (0.26 mL, 4.41 mmol) was added to the above mixture and stirred at 0° C. for 10 min. And then MeI (0.3 mL, 4.41 mmol) was added to the above mixture at 0° C. The resulting mixture was stirred at room temperature for 1 hour. The reaction mixture was quenched by the addition of water and extracted with ethyl acetate. The combined organic layers were washed with brine, dried over anhydrous sodium sulfate, filtered, and concentrated under vacuum to afford S-methyl O-(2-methyltetrahydrofuran-3-yl) carbonodithioate (534.0 mg, crude) as a yellow oil. MS (ESI) calc'd for (C₇H₁₂O₂S₂) (M+1)⁺, 193.0.

### Step 2 O-(2-methyltetrahydrofuran-3-yl) hydrazinecarbothioate

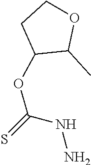

To a stirred solution of S-methyl O-(2-methyltetrahydrofuran-3-yl) carbonodithioate (530.0 mg. 2.76 mmol) in Methanol (5 mL) was added hydrazine (121.2 mg, 3.03 mmol, 80%) at 20° C. The resulting solution was stirred at 20° C. for 30 min. The organic solvent was removed under vacuum to afford O-(2-methyltetrahydrofuran-3-yl) hydrazinecarbothioate (460.0 mg, crude) as a yellow oil. MS (ESI) calc'd for (C₆H₁₂N₂O₂S) (M+1)⁺, 177.1. found 177.0.

### Step 3 5-((2-methyltetrahydrofuran-3-yl)oxy)-1,3,4-thiadiazol-2-amine

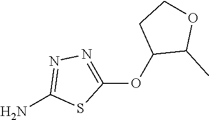

To a stirred solution of O-(2-methyltetrahydrofuran-3-yl) hydrazinecarbothioate (460.0 mg, 2.61 mmol) in Methanol (2.5 mL) were added TEA (0.7 mL, 5.22 mmol) and BrCN (301.0 mg, 2.87 mmol) at 20° C. The resulting solution was stirred at 20° C. for 1 hr. The reaction mixture was quenched with water and extracted with ethyl acetate. The combined organic layers were washed with brine, dried over anhydrous sodium sulfate, filtered, and concentrated under vacuum. The resulting residue was dissolved in MeOH/DCM (0.2/2 mL) and purified by Combi Flash (Biotage Isolera Prime) which applied to a 40 g silica gel column that was eluted with 0˜20% methanol in dichloromethane within 30 min to afford 5-((2-methyltetrahydrofuran-3-yl)oxy)-1,3,4-thiadiazol-2-amine (144.0 mg, 27%) as a yellow solid. MS (ESI) calc'd for (C₇H₁₁N₃O₂S) (M+1)⁺, 202.1. found 202.1.

### Step 4 2′-chloro-5′-methoxy-6-methyl-N-(5-((2-methyltetrahydrofuran-3-yl)oxy)-1,3,4-thiadiazol-2-yl)-(4,4′-bipyridine)-3-carboxamide

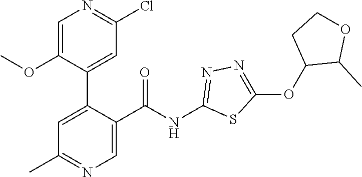

To a stirred solution of 5-((2-methyltetrahydrofuran-3-yl)oxy)-1,3,4-thiadiazol-2-amine (120.0 mg, 0.59 mmol) in Acetonitrile (1 mL) were added Intermediate G (166.0 mg, 0.59 mmol) and 1-methylimidazole (0.23 mL, 2.98 mmol) at 20° C. To the above solution was added a solution of TCFH (168.0 mg, 0.59 mmol) in Acetonitrile (0.5 mL) at 20° C. under nitrogen. The resulting mixture was then stirred at 20° C. for 2 hr. The resulting mixture was dissolved in DMF (3 mL) which was applied to a 20 g C18 column and purified by Combi Flash (Biotage Isolera Prime), eluted with 5˜80% acetonitrile in water within 45 min to afford 2′-chloro-5′-methoxy-6-methyl-N-(5-((2-methyltetrahydrofuran-3-yl)oxy)-1,3,4-thiadiazol-2-yl)-(4,4′-bipyridine)-3-carboxamide (181.6 mg, 65%) as a white solid. MS (ESI) calc'd for (C₂₀H₂₀ClN₅O₄S) (M+1)⁺, 462.1. found, 462.2.

### Step 5 2′-chloro-5′-methoxy-6-methyl-N-(5-(((2r,3r)-2-methyltetrahydrofuran-3-yl)oxy)-1,3,4-thiadiazol-2-yl)-(4,4′-bipyridine)-3-carboxamide and 2′-chloro-5′-methoxy-6-methyl-N-(5-(((2r,3s)-2-methyltetrahydrofuran-3-yl)oxy)-1,3,4-thiadiazol-2-yl)-(4,4′-bipyridine)-3-carboxamide

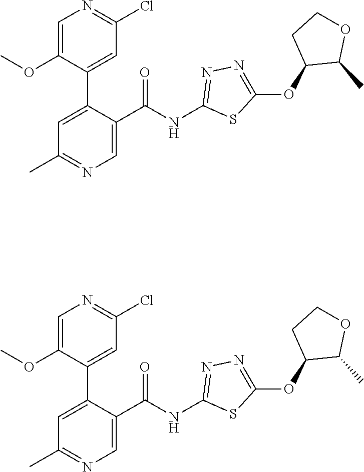

A mixture of 2′-chloro-5′-methoxy-6-methyl-N-(5-((2-methyltetrahydrofuran-3-yl)oxy)-1,3,4-thiadiazol-2-yl)-(4,4′-bipyridine)-3-carboxamide (181.6 mg) was separated by prep-HPLC with the following condition: (Column: Xselect CSH C18 OBD Column 30*150 mm 5 μm, n; Mobile Phase A: Water (0.1% FA), Mobile Phase B: ACN; Flow rate: 60 mL/min; Gradient: 28% B to 32% B in 12 min, 32% B to 95% B in 12.2 min, 95% B to 95% B in 13.7 min, 95% B to 5% B in 15 min, 5% B; Wave Length: 220 nm; RT1 (min): 8.92, 11.35 (min); Injection Volume: 1.2 mL; Number Of Runs: 2) to afford 2′-chloro-5′-methoxy-6-methyl-N-(5-(((2r,3r)-2-methyltetrahydrofuran-3-yl)oxy)-1,3,4-thiadiazol-2-yl)-(4,4′-bipyridine)-3-carboxamide (40.7 mg. 22%) as a white solid with shorter retention time on prep-HPLC and 2′-chloro-5′-methoxy-6-methyl-N-(5-(((2r,3s)-2-methyltetrahydrofuran-3-yl)oxy)-1,3,4-thiadiazol-2-yl)-(4,4′-bipyridine)-3-carboxamide (51.6 mg, 28%) as a white solid with longer retention time on prep-HPLC. The absolute stereochemistry was not determined and was arbitrarily assigned.

2′-chloro-5′-methoxy-6-methyl-N-(5-(((2r,3r)-2-methyltetrahydrofuran-3-yl)oxy)-1,3,4-thiadiazol-2-yl)-(4,4′-bipyridine)-3-carboxamide: MS (ESI) calc'd for (C₂₀H₂₀ClN₅O₄S) (M+1)⁺, 462.1. found, 462.1. ¹H NMR (400 MHz, DMSO-d₆) δ 12.92 (s, 1H), 8.82 (s, 1H), 8.17 (s, 1H), 7.53 (s, 1H), 7.41 (s, 1H), 5.40 (s, 1H), 3.98-3.87 (m, 2H), 3.70-3.34 (m, 4H), 2.58 (s, 3H), 2.47-2.34 (m, 1H), 2.15-2.04 (m, 1H), 1.19 (d, J=6.4 Hz, 3H). 2′-chloro-5′-methoxy-6-methyl-N-(5-(((2r,3s)-2-methyltetrahydrofuran-3-yl)oxy)-1,3,4-thiadiazol-2-yl)-(4,4′-bipyridine)-3-carboxamide: MS (ESI) calc'd for (C₂₀H₂₀ClN₅SO₄S) (M+1)⁺, 462.1. found, 462.1. ¹H NMR (400 MHz, DMSO-d₆) δ 12.90 (s, 1H), 8.81 (s, 1H), 8.18 (s, 1H), 7.54 (s, 1H), 7.44 (s, 1H), 5.14-5.07 (m, 1H), 4.17-4.07 (m, 1H), 3.99-3.89 (m, 1H), 3.84-3.74 (m, 1H), 3.64 (s, 3H), 2.60 (s, 3H), 2.39-2.25 (m, 1H), 2.15-2.05 (m, 1H), 1.21 (d, J=6.4 Hz, 3H).

### Example 95, 228 and 117

### N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide, (S)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide and (R)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide

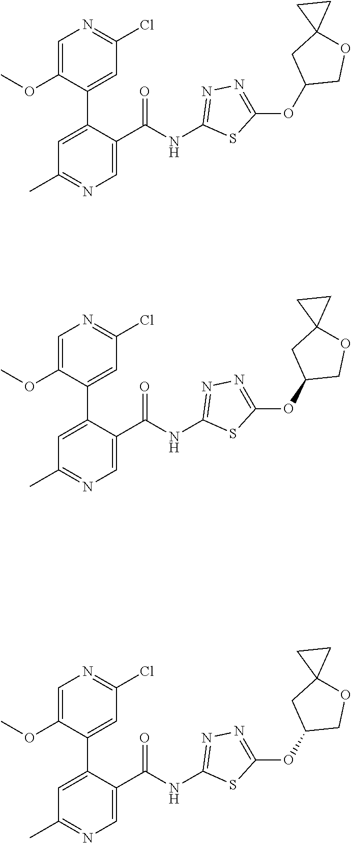 [SMILES: COc1cnc(Cl)cc1-c1cc(C)ncc1C(=O)Nc1nnc(OC2COC3(CC3)C2)s1 ; COc1cnc(Cl)cc1-c1cc(C)ncc1C(=O)Nc1nnc(O[C@@H]2COC3(CC3)C2)s1 ; COc1cnc(Cl)cc1-c1cc(C)ncc1C(=O)Nc1nnc(O[C@H]2COC3(CC3)C2)s1]

### Step-1: S-methyl O-(4-oxaspiro(2.4)heptan-6-yl) carbonodithioate

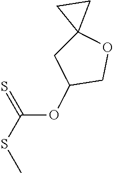

To a stirred solution of 4-oxaspiro(2.4)heptan-6-ol (100.0 mg, 0.87 mmol) in Tetrahydrofuran (3 mL) was added NaH (42.0 mg, 1.05 mmol, 60%) at 0° C. The reaction mixture was stirred at 0° C. for 0.5 h. Then CS₂ (100.0 mg, 1.31 mmol) was added to the above mixture at 0° C. The resulting solution was then stirred at 0° C. for 0.5 h. Then MeI (187.0 mg, 1.31 mmol) was added to the above mixture at 0° C. The resulting solution was then stirred at 0° C. for 1 h. The reaction mixture was quenched by the addition of water and extracted with ethyl acetate. The combined organic layers were washed with brine, dried over anhydrous sodium sulfate, filtered, and concentrated under vacuum to afford S-methyl O-(4-oxaspiro(2.4)heptan-6-yl) carbonodithioate (150.0 mg, crude) as yellow oil. The crude product was used in next step without further purification.

### Step-2: O-(4-oxaspiro(2.4)heptan-6-yl) hydrazinecarbothioate

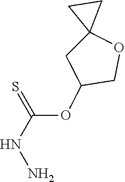 [SMILES: NNC(=S)OC1COC2(CC2)C1]

To a stirred solution of S-methyl O-(4-oxaspiro(2.4)heptan-6-yl) carbonodithioate (150.0 mg, 0.73 mmol) in Methanol (3 mL) was added hydrazine (47.1 mg, 0.73 mmol) at 25° C. The reaction mixture was stirred at 25° C. for 1 h. The organic solvent was removed under vacuum to afford O-(4-oxaspiro(2.4)heptan-6-yl) hydrazinecarbothioate (130.0 mg, crude) as yellow oil, which was used in the next step without further purification.

### Step-3: 5-((4-oxaspiro(2.4)heptan-6-yl)oxy)-1,3,4-thiadiazol-2-amine

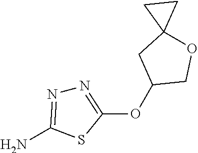 [SMILES: Nc1nnc(OC2COC3(CC3)C2)s1]

To a stirred solution of O-(4-oxaspiro(2.4)heptan-6-yl) hydrazinecarbothioate (130.0 mg, 0.69 mmol), cyanic bromide (110.0 mg, 1.03 mmol) and Et₃N (175.0 mg, 1.72 mmol) in Methanol (3 mL) was stirred at 25° C. for 2 h under nitrogen atmosphere. The organic solvent was removed under vacuum. The resulting residue was dissolved in MeOH (2 mL) which was applied to a 20 g C18 column and purified by Combi Flash (Biotage Isolera Prime), eluted with 5˜17% acetonitrile in water within 20 min to afford 5-((4-oxaspiro(2.4)heptan-6-yl)oxy)-1,3,4-thiadiazol-2-amine (80.0 mg, 43% over three steps) as a white solid. MS (ESI) calc'd for (C₈H₁₁N₃O₂S) (M+1)⁺, 214.1. found 214.1.

### Step-4: (S)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide and (R)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide

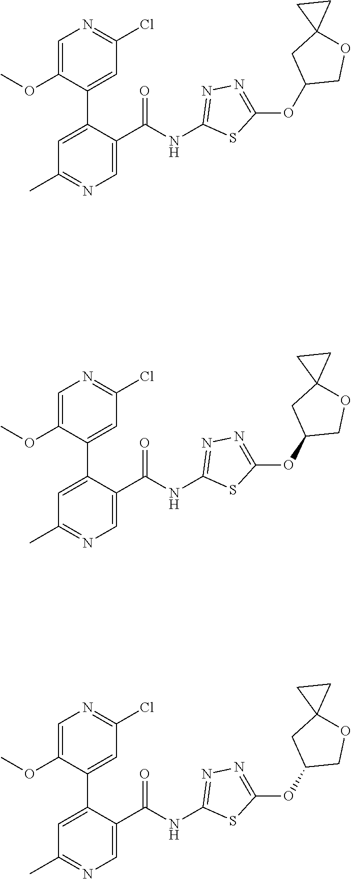 [SMILES: COc1cnc(Cl)cc1-c1cc(C)ncc1C(=O)Nc1nnc(OC2COC3(CC3)C2)s1 ; COc1cnc(Cl)cc1-c1cc(C)ncc1C(=O)Nc1nnc(O[C@@H]2COC3(CC3)C2)s1 ; COc1cnc(Cl)cc1-c1cc(C)ncc1C(=O)Nc1nnc(O[C@H]2COC3(CC3)C2)s1]

To a stirred solution of Intermediate G (92.0 mg, 0.33 mmol) in Acetonitrile (5 mL) were sequentially added 5-((4-oxaspiro(2.4)heptan-6-yl)oxy)-1,3,4-thiadiazol-2-amine (70.4 mg, 0.33 mmol), 1-methyl-1H-imidazole (136.0 mg, 1.65 mmol) and N,N,N′,N′-Tetramethylchloroformamidinium hexafluorophosphate (93.0 mg, 0.33 mmol) at 25° C. The resulting solution was stirred at 25° C. for 2 h. The organic solvent was removed under vacuum. The resulting residue was dissolved in ACN (2 mL) was applied to a 20 g C18 column and purified by Combi Flash (Biotage Isolera Prime), eluted with 5˜40% acetonitrile in water within 35 min to afford racemic N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide (65.0 mg, 38%) as a yellow solid. The racemic compound was separated by prep-chiral HPLC with the following condition: (Column: CHIRAL ART Cellulose-SC, 2*25 cm, 5 μm; Flow rate: 20 mL/min; Gradient: 30% B to 30% B in 16 min; Wave Length: 220/254 nm; RT1 (min): 12.80; RT2 (min): 14.92; Sample Solvent: MeOH:DCM=1:1; Injection Volume: 0.3 mL; Number Of Runs: 8) to afford (S)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide (14.3 mg, 21%) as a white solid with the first peak on chiral HPLC and (S)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide (21.1 mg, 32%) as a yellow solid with the second peak on chiral HPLC. The absolute stereochemistry was determined using vibrational circular dichroism (VCD) and ab initio calculations.

(S)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide: MS (ESI) calc'd for (C₂₁H₂₀ClN₅O₄S) (M+1)⁺, 474.1. found, 474.1. ¹H NMR (400 MHz, DMSO-d₆) δ 12.92 (s, 1H), 8.83 (s, 1H), 8.17 (s, 1H), 7.51 (s, 1H), 7.40 (s, 1H), 5.62-5.54 (m, 1H), 4.10-3.94 (m, 2H), 3.64 (s, 3H), 2.58 (s, 3H), 2.50-2.42 (m, 1H), 2.19-2.11 (m, 1H), 0.87-0.77 (m, 1H), 0.77-0.68 (m, 1H), 0.66-0.56 (m, 1H), 0.56-0.46 (m, 1H).

(R)—N-(5-((4-oxaspiro(2.4)heptan-6-yl)oxy)-1,3,4-thiadiazol-2-yl)-2′-chloro-5′-methoxy-6-methyl-(4,4′-bipyridine)-3-carboxamide: MS (ESI) calc'd for (C₂₁H₂₀ClN₅O₄S) (M+1)⁺, 474.1. found, 474.1. ¹H NMR (400 MHz, DMSO-d₆) δ 12.96 (s, 1H), 8.82 (s, 1H), 8.17 (s, 1H), 7.51 (s, 1H), 7.40 (s, 1H), 5.62-5.55 (m, 1H), 4.09-3.93 (m, 2H), 3.63 (s, 3H), 2.58 (s, 3H), 2.51-2.42 (m, 1H), 2.19-2.10 (m, 1H), 0.88-0.78 (m, 1H), 0.77-0.70 (m, 1H), 0.66-0.56 (m, 1H), 0.56-0.46 (m, 1H).

### Example 120

### 7-(2-fluoro-6-methoxyphenyl)-N-(5-methoxy-1,3,4-thiadiazol-2-yl)-3-methylimidazo(1,2-a)pyridine-6-carboxamide

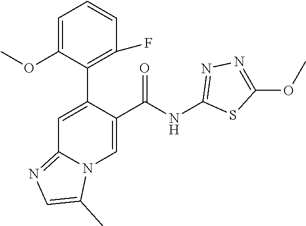

### Step-1: methyl 6-amino-4-(2-fluoro-6-methoxyphenyl)nicotinate

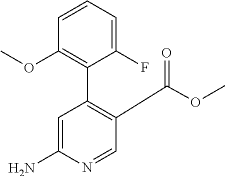

To a solution of methyl 6-amino-4-bromonicotinate (200.0 mg, 0.86 mmol) in 1,4-Dioxane (5 mL) and water (1 mL) were added (2-fluoro-6-methoxyphenyl)boronic acid (177.0 mg, 1.03 mmol), Pd(dtbpf)Cl₂ (56.4 mg, 0.087 mmol) and potassium phosphate (367.0 mg, 1.73 mmol) at 20° C. under nitrogen. The resulting solution was stirred at 90° C. for 2 h under nitrogen. The solvent was removed under vacuum, the residue was applied to a 40 g C18 column and purified by Combi Flash (Biotage Isolera Prime), eluted with 5˜40% acetonitrile in water within 40 min to afford methyl 6-amino-4-(2-fluoro-6-methoxyphenyl)nicotinate (180.0 mg, 62%) as a brown solid. MS (ESI) calc'd for (C₁₄H₁₃FN₂O₃) (M+1)⁺, 277.1. found 277.1.

### Step-2: methyl 7-(2-fluoro-6-methoxyphenyl)-3-methylimidazo(1,2-a)pyridine-6-carboxylate

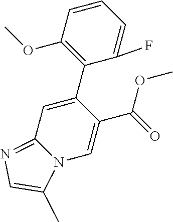

A mixture of methyl 6-amino-4-(2-fluoro-6-methoxyphenyl)nicotinate (230.0 mg, 0.66 mmol) and 1-bromopropan-2-one (109.0 mg, 0.79 mmol) in Ethanol (4 mL) was stirred at 60° C. for 16 h. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (3 mL) which was applied to a 40 g C18 column and purified by Combi Flash (Biotage Isolera Prime), eluted with 5˜45% acetonitrile in water within 40 min to afford methyl 7-(2-fluoro-6-methoxyphenyl)-3-methylimidazo(1,2-a)pyridine-6-carboxylate (100.0 mg, 46%) as a red solid. MS (ESI) calc'd for (C₁₇H₁₅FN₂O₃) (M+1)⁺, 315.3. found 315.2.

### Step-3: 7-(2-fluoro-6-methoxyphenyl)-3-methylimidazo(1,2-a)pyridine-6-carboxylic Acid

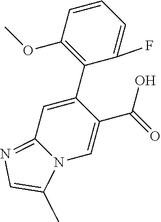 [SMILES: COc1cccc(F)c1-c1cc2ncc(C)n2cc1C(=O)O]

To a solution of methyl 7-(2-fluoro-6-methoxyphenyl)-3-methylimidazo(1,2-a)pyridine-6-carboxylate (100.0 mg, 0.32 mmol) in Methanol (1 mL) was added NaOH (38.2 mg, 0.95 mmol) in Water (1 mL). The resulting solution was stirred at 50° C. for 3 h. The aqueous layer was acidified with Citric acid to pH ˜4 and extracted with ethyl acetate. The combined organic layers were washed with brine, dried over anhydrous sodium sulfate, filtered, and concentrated under vacuum. The resulting crude residue was applied to a 20 g C18 column and purified by Combi Flash (Biotage Isolera Prime), eluted with 5˜14% acetonitrile in water within 30 min to afford 7-(2-fluoro-6-methoxyphenyl)-3-methylimidazo(1,2-a)pyridine-6-carboxylic acid (50.0 mg, 52%) as a white solid. MS (ESI) calc'd for (C₁₆H₁₃FN₂O₃) (M+1)⁺, 301.1. found 301.1.

### Step-4: 7-(2-fluoro-6-methoxyphenyl)-N-(5-methoxy-1,3,4-thiadiazol-2-yl)-3-methylimidazo(1,2-a)pyridine-6-carboxamide

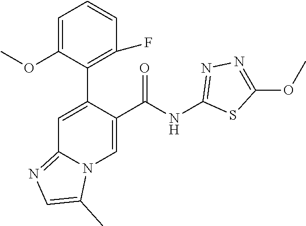

To a stirred solution of 7-(2-fluoro-6-methoxyphenyl)-3-methylimidazo(1,2-a)pyridine-6-carboxylic acid (20.0 mg, 0.067 mmol) in DMF (1 mL) was added HATU (38.0 mg, 0.10 mmol) at 20° C. The resulting solution was stirred at 20° C. for 10 min. To the above solution was added 5-methoxy-1,3,4-thiadiazol-2-amine (8.8 mg, 0.067 mmol) and N,N-diisopropylethylamine (17.2 mg, 0.13 mmol). The resulting solution was stirred at 20° C. for 1 h. The resulting residue was applied to a 20 g C18 column and purified by Combi Flash (Biotage Isolera Prime), eluted with 5˜80% acetonitrile in water within 30 min to afford 7-(2-fluoro-6-methoxyphenyl)-N-(5-methoxy-1,3,4-thiadiazol-2-yl)-3-methylimidazo(1,2-a)pyridine-6-carboxamide (4.8 mg, 17%) as a white solid. MS (ESI) calc'd for (C₁₉H₁₆FN₅O₃S) (M+1)⁺, 414.1. found, 414.1. ¹H NMR (400 MHz, DMSO-d₆) δ 12.90 (s, 1H), 9.35 (d, J=1.6 Hz, 1H), 7.89 (d, J=1.2 Hz, 1H), 7.68 (d, J=1.6 Hz, 1H), 7.54-7.44 (m, 1H), 7.03 (d, J=8.4 Hz, 1H), 6.99-6.90 (m, 1H), 4.10 (s, 3H), 3.72 (s, 3H), 2.31 (s, 3H).

### Example 121

### 5-(2-fluoro-6-methoxyphenyl)-2-(hydroxymethyl)-N-(5-methoxy-1,3,4-thiadiazol-2-yl)isonicotinamide

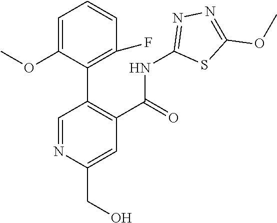

### Step-1: methyl 2,5-dichloroisonicotinate

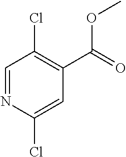

To a stirred solution of 2,5-dichloroisonicotinic acid (10.0 g, 52.00 mmol) in Dichloromethane (100.0 mL) were added Methanol (10.0 mL) and (diazomethyl)trimethylsilane (52 mL, 104.20 mmol) at 0° C. The resulting solution was stirred at 0° C. for 2 h. The organic solvent was removed under vacuum. The resulting residue was dissolved in Dichloromethane (10 mL) and purified by Combi Flash (Biotage Isolera Prime) which applied to a 330 g silica gel column that was eluted with 0˜38% ethyl acetate in petroleum ether within 40 min to afford methyl 2,5-dichloroisonicotinate (9.3 g, 83%) as colorless oil. MS (ESI) calc'd for (C₇H₅ClNO₂) (M+1)⁺, 206.0. found 206.0.

### Step-2: methyl 5-chloro-2-vinylisonicotinate

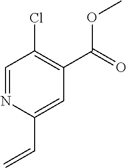

To a stirred solution of methyl 2,5-dichloroisonicotinate (4.8 g, 23.35 mmol) and trifluoro(vinyl)-14-borane, potassium salt (3.1 g, 23.35 mmol) in 1,4-Dioxane (50.0 mL) were added K₂CO₃ (9.7 g. 70.00 mmol) in Water (5.0 mL) and PdCl₂(dppf) (1.7 g, 2.35 mmol) at 25° C. under nitrogen atmosphere. The resulting solution was stirred at 80° C. for 2 h under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was dissolved in Dichloromethane (9.0 mL) and purified by Combi Flash (Biotage Isolera Prime) which applied to a 120 g silica gel column that was eluted with 0˜35% ethyl acetate in petroleum ether within 40 min to afford methyl 5-chloro-2-vinylisonicotinate (3.4 g, 73%) as colorless oil. MS (ESI) calc'd for (C₉H₈ClNO₂) (M+1)⁺, 198.0. found 198.0.

### Step-3: methyl 5-chloro-2-formylisonicotinate

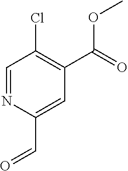

To a stirred solution of methyl 5-chloro-2-vinylisonicotinate (2.7 g, 13.66 mmol) in Tetrahydrofuran (15.0 mL) and Water (15.0 mL) were added osmium tetroxide (0.9 mL, 2.73 mmol) and sodium periodate (6.9 g, 27.3 mmol) at 25° C. The resulting solution was stirred at 25° C. for 2 h. The reaction mixture was quenched by the addition of water and extracted with ethyl acetate. The combined organic layers were washed with brine, dried over anhydrous sodium sulfate, filtered, and concentrated under vacuum. The resulting residue was dissolved in Dichloromethane (10.0 mL) and purified by Combi Flash (Biotage Isolera Prime) which applied to a 120 g silica gel column that was eluted with 0˜35% ethyl acetate in petroleum ether within 40 min to afford methyl 5-chloro-2-formylisonicotinate (1.6 g, 56%) as colorless oil. MS (ESI) calc'd for (C₈H₆ClNO₃) (M+1)⁺, 200.0. found 200.0.

### Step-4: methyl 5-chloro-2-(hydroxymethyl)isonicotinate

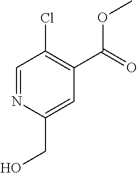

To a stirred solution of methyl 5-chloro-2-formylisonicotinate (1.2 g, 6.01 mmol) in Methanol (10.0 mL) was added NaBH₄ (0.1 g, 3.01 mmol) at 0° C. The resulting solution was stirred at 0° C. for 15 min. The reaction mixture was quenched by the addition of water and extracted with ethyl acetate. The combined organic layers were washed with brine, dried over anhydrous sodium sulfate, filtered, and concentrated under vacuum to afford methyl 5-chloro-2-(hydroxymethyl)isonicotinate (840 mg, crude) as a yellow solid. MS (ESI) calc'd for (C₈H₈ClNO₃) (M+1)⁺, 202.0. found 202.0.

### Step-5: methyl 5-(2-fluoro-6-methoxyphenyl)-2-(hydroxymethyl)isonicotinate

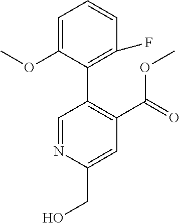

To a degassed solution of methyl 5-chloro-2-(hydroxymethyl)isonicotinate (1.0 g, 5.16 mmol) in 1,4-Dioxane (8.0 mL) and Water (1.6 mL) were added (2-fluoro-6-methoxyphenyl)boronic acid (1.7 g, 10.32 mmol) and K₂CO₃ (2.2 g, 15.48 mmol), 1,1′-Bis (di-t-butylphosphino)ferrocene palladium dichloride (1.0 g, 1.55 mmol) at 23° C. under nitrogen. The resulting solution was stirred at 80° C. for 2 h under nitrogen. The suspension was filtered with diatomite. The filtrate was collected and concentrated under vacuum. The resulting residue was purified by Combi Flash (Biotage Isolera Prime) which applied to 80 g silica gel column and eluted with 0˜75% ethyl acetate in petroleum ether within 45 min to afford methyl 5-(2-fluoro-6-methoxyphenyl)-2-(hydroxymethyl)isonicotinate (950 mg, 61%) as a white solid. MS (ESI) calc'd for (C₁₅H₁₄FNO₄) (M+1)⁺, 292.1. found 292.1.

### Step-6: 5-(2-fluoro-6-methoxyphenyl)-2-(hydroxymethyl)isonicotinic Acid

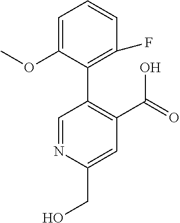 [SMILES: COc1cccc(F)c1-c1cnc(CO)cc1C(=O)O]

To a stirred solution of methyl 5-(2-fluoro-6-methoxyphenyl)-2-(hydroxymethyl)isonicotinate (500.0 mg, 1.71 mmol) in Tetrahydrofuran (3.0 mL) were added LiOH (164.0 mg, 6.87 mmol) and Water (1.0 mL) at 25° C. The resulting solution was stirred at 25° C. for 1 h. The organic solvent was removed under vacuum. The aqueous layer was acidified with citric acid to pH ˜4 and extracted with ethyl acetate. The combined organic layers were washed with brine, dried over anhydrous sodium sulfate, filtered, and concentrated under vacuum to afford 5-(2-fluoro-6-methoxyphenyl)-2-(hydroxymethyl)isonicotinic acid (250 mg, crude) as a white solid. MS (ESI) calc'd for (C₁₄H₁₂FNO₄) (M+1)⁺, 278.1. found 278.1.

### Step-7: 5-(2-fluoro-6-methoxyphenyl)-2-(hydroxymethyl)-N-(5-methoxy-1,3,4-thiadiazol-2-yl)isonicotinamide

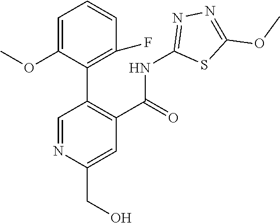

To a stirred solution of 5-(2-fluoro-6-methoxyphenyl)-2-(hydroxymethyl)isonicotinic acid (100 mg, 0.36 mmol) in Acetonitrile (1 mL) were sequentially added 5-methoxy-1,3,4-thiadiazol-2-amine (47 mg, 0.36 mmol) and 1-methyl-1H-imidazole (148 mg, 1.80 mmol) at 23° C. Then N,N,N′,N′-Tetramethylchloroformamidinium hexafluorophosphate (101 mg, 0.36 mmol) in acetonitrile (1 mL) was added to the above mixture at 25° C. The resulting solution was stirred at 25° C. for 2 h. The reaction mixture (2 mL) was purified by prep-HPLC with the following condition: (Column: XBridge Shield RP18 OBD Column, 19*150 mm, 5 μm; Mobile Phase A: Water (10 mmol/L NH4HCO3), Mobile Phase B: ACN; Flow rate: 25 mL/min; Gradient: 15% B to 35% B in 8 min, 35% B; Wave Length: 254 nm) to afford 5-(2-fluoro-6-methoxyphenyl)-2-(hydroxymethyl)-N-(5-methoxy-1,3,4-thiadiazol-2-yl)isonicotinamide (43.7 mg, 30%) as a white solid. MS (ESI) calc'd for (C₁₇H₁₅FN₄O₄S) (M+1)⁺, 391.1. found 391.1. ¹H NMR (400 MHz, DMSO-d₆) δ 12.99 (br, 1H), 8.49 (s, 1H), 7.81 (s, 1H), 7.44-7.34 (m, 1H), 6.96-6.85 (m, 2H), 5.60 (t, J=5.6 Hz, 1H), 4.68 (d, J=5.6 Hz, 2H), 4.06 (s, 3H), 3.58 (s, 3H).

### Example 123

### 4-((1,2,4)triazolo(1,5-a)pyridin-8-yl)-N-(5-methoxy-1,3,4-thiadiazol-2-yl)-6-methylnicotinamide

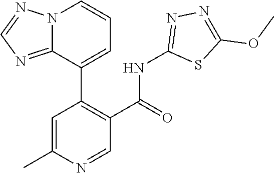 [SMILES: COc1nnc(NC(=O)c2cnc(C)cc2-c2cccn3ncnc23)s1]

### Step-1: methyl 4-((1,2,4)triazolo(1,5-a)pyridin-8-yl)-6-methylnicotinate

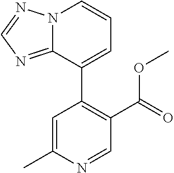 [SMILES: COC(=O)c1cnc(C)cc1-c1cccn2ncnc12]

To a stirred solution of methyl 6-methyl-4-(4,4,5,5-tetramethyl-1,3,2-dioxaborolan-2-yl)nicotinate (1.0 g, 3.79 mmol) in 1,4-Dioxane (10 mL) were added 8-bromo-(1,2,4)triazolo(1,5-a)pyridine (500.0 mg, 2.52 mmol), K₂CO₃ (1.0 g, 7.57 mmol) in Water (2 mL) and Pd(dtbpf)Cl₂ (164.0 mg, 0.25 mmol) at 20° C. The resulting solution was stirred at 80° C. for 1 h under nitrogen atmosphere. The reaction mixture was diluted with water, extracted with ethyl acetate. The combined organic layers were washed with brine, dried over anhydrous sodium sulfate, filtered, and concentrated under vacuum. The resulting residue was dissolved in DCM (2 mL) and purified by Combi Flash (Biotage Isolera Prime) which applied to a 40 g silica gel column that was eluted with 0˜100% ethyl acetate in petroleum ether within 30 min to afford methyl 4-((1,2,4)triazolo(1,5-a)pyridin-8-yl)-6-methylnicotinate (500.0 mg, 70%) as a yellow solid. MS (ESI) calc'd for (C₁₄H₁₂N₄O₂) (M+1)⁺, 269.1. found, 269.1.

### Step-2: 4-((1,2,4)triazolo(1,5-a)pyridin-8-yl)-6-methylnicotinic Acid

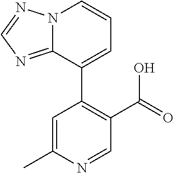 [SMILES: Cc1cc(-c2cccn3ncnc23)c(C(=O)O)cn1]

To a stirred solution of methyl 4-((1,2,4)triazolo(1,5-a)pyridin-8-yl)-6-methylnicotinate (380.0 mg, 1.41 mmol) in Tetrahydrofuran (2 mL) and Water (2 mL) was added LiOH (67.8 mg, 2.83 mmol) at 20° C. The resulting solution was stirred at 25° C. for 1 h. The mixture was acidified with citric acid to pH ˜3. The resulting residue was dissolved in DMF (1 mL) which was applied to a 20 g C18 column, purified by Combi Flash (Biotage Isolera Prime) and eluted with 5˜30% acetonitrile in water within 20 min to afford 4-((1,2,4)triazolo(1,5-a)pyridin-8-yl)-6-methylnicotinic acid (256.0 mg, 70%) as a white solid. MS (ESI) calc'd for (C₁₃H₁₀N₄O₂) (M+1)⁺, 255.1. found, 255.1.

### Step-3: 4-((1,2,4)triazolo(1,5-a)pyridin-8-yl)-N-(5-methoxy-1,3,4-thiadiazol-2-yl)-6-methylnicotinamide

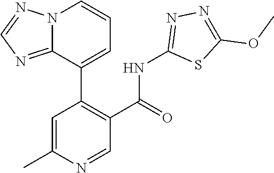 [SMILES: COc1nnc(NC(=O)c2cnc(C)cc2-c2cccn3ncnc23)s1]

To a stirred solution of 4-((1,2,4)triazolo(1,5-a)pyridin-8-yl)-6-methylnicotinic acid (100.0 mg, 0.39 mmol) in Ethyl acetate (2 mL) was added 5-methoxy-1,3,4-thiadiazol-2-amine (51.6 mg, 0.39 mmol) at 20° C. To the above solution were added pyridine (0.06 mL, 0.78 mmol) and T₃P (501.0 mg, 0.78 mmol, 50% in EtOAc) at 0° C. under nitrogen. The resulting solution was stirred at 80° C. for 3 h. The reaction mixture was concentrated under vacuum. The resulting residue was dissolved in DMSO (1.5 mL) which was applied to a 20 g C18 column and purified by Combi Flash (Biotage Isolera Prime), eluted with 5˜100% acetonitrile in water within 25 min to afford 4-((1,2,4)triazolo(1,5-a)pyridin-8-yl)-N-(5-methoxy-1,3,4-thiadiazol-2-yl)-6-methylnicotinamide (48.8 mg, 33%) as a light brown solid. MS (ESI) calc'd for (C₁₆H₁₃N₇O₂S) (M+1)⁺, 368.1. found, 368.1. ¹H NMR (400 MHz, DMSO-d₆) δ 13.00 (s, 1H), 9.01 (d, J=4.0 Hz, 1H), 8.88 (s, 1H), 8.39 (s, 1H), 7.76 (d, J=8.0 Hz, 1H), 7.60 (s, 1H), 7.33 (t, J=8.0 Hz, 1H), 4.04 (s, 3H), 2.63 (s, 3H).

### Example 126

### 4-(2,3-dihydropyrazolo(5,1-b)oxazol-7-yl)-N-(5-methoxy-1,3,4-thiadiazol-2-yl)-6-methylnicotinamide

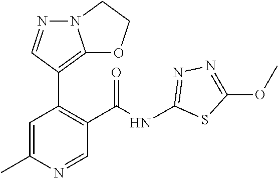

### Step-1: 2,3-dihydropyrazolo(5,1-b)oxazole

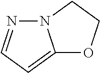

To a solution of 1,2-dihydro-3H-pyrazol-3-one (5.0 g, 59.50 mmol) in Acetonitrile (80 mL) were added 1,2-dibromoethane (33.5 g, 178.50 mmol) and K₂CO₃ (24.6 g, 178.50 mmol). The above solution was stirred at 90° C. for 16 h. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (3 mL) which was applied to a 120 g C18 column, purified by Combi Flash (Biotage Isolera Prime) and eluted with 5˜90% acetonitrile in water within 35 min to afford 2,3-dihydropyrazolo(5,1-b)oxazole (1.2 g, 14%) as a white solid. MS (ESI) calc'd for (C₅H₆N₂O) (M+1)⁺, 111.0. found, 111.0.

### Step-2: 7-bromo-2,3-dihydropyrazolo(5,1-b)oxazole

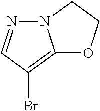

To a stirred solution of 2,3-dihydropyrazolo(5,1-b)oxazole (1.2 g, 8.50 mmol) in Acetonitrile (15 mL) was added NBS (1.8 g, 10.11 mmol) at 0° C. under nitrogen atmosphere. The resulting solution was stirred at 0° C. for 2 hr under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (1 mL) which was applied to a 40 g C18 column, purified by Combi Flash (Biotage Isolera Prime) and eluted with 5˜85% acetonitrile in water within 30 min to afford 7-bromo-2,3-dihydropyrazolo(5,1-b)oxazole (840.0 mg, 47%) as a brown solid. MS (ESI) calc'd for (C₅H₅BrN₂O) (M+1)⁺, 189.0. found, 189.0.

### Step-3: methyl 4-(2,3-dihydropyrazolo(5,1-b)oxazol-7-yl)-6-methylnicotinate

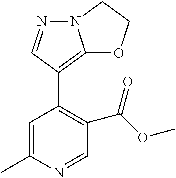

To a stirred solution of methyl 6-methyl-4-(4,4,5,5-tetramethyl-1,3,2-dioxaborolan-2-yl)nicotinate (440.0 mg, 1.59 mmol) in 1,4-Dioxane (3 mL) were sequentially added 7-bromo-2,3-dihydropyrazolo(5,1-b)oxazole (300.0 mg, 1.59 mmol), K₂CO₃ (439.0 mg, 3.17 mmol) and Pd(dppf)Cl₂ (116.0 mg, 0.16 mmol) at 23° C. The resulting solution was stirred at 80° C. for 2 hr under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (1 mL) and purified by Combi Flash (Biotage Isolera Prime) which applied to a 40 g silica gel column that was eluted with 0˜50% ethyl acetate in petroleum ether within 25 min to afford methyl 4-(2,3-dihydropyrazolo(5,1-b)oxazol-7-yl)-6-methylnicotinate (210.0 mg, 50%) as a yellow solid. MS (ESI) calc'd for (C₁₃H₁₃N₃O₃) (M+1)⁺, 260.1. found, 260.1.

### Step-4: 4-(2,3-dihydropyrazolo(5,1-b)oxazol-7-yl)-6-methylnicotinic Acid

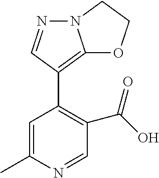 [SMILES: Cc1cc(-c2cnn3c2OCC3)c(C(=O)O)cn1]

To a stirred solution of methyl 4-(2,3-dihydropyrazolo(5,1-b)oxazol-7-yl)-6-methylnicotinate (200.0 mg, 0.77 mmol) in Water (0.3 mL) and THF (1 mL) was added LiOH (162.0 mg, 3.86 mmol) at 25° C. The reaction mixture was stirred at 25° C. for 16 h under nitrogen atmosphere. The resulting residue was acidified to pH ˜5 with formic acid, and then diluted with DMF (1 mL) which was applied to a 25 g C18 column, purified by Combi Flash (Biotage Isolera Prime) and eluted with 5˜30% acetonitrile in water within 20 min to afford 4-(2,3-dihydropyrazolo(5,1-b)oxazol-7-yl)-6-methylnicotinic acid (90.0 mg, 40%) as a yellow solid. MS (ESI) calc'd for (C₁₂H₁₁N₃O3) (M+1)⁺, 246.1. found, 246.1.

### Step-5: 4-(2,3-dihydropyrazolo(5,1-b)oxazol-7-yl)-N-(5-methoxy-1,3,4-thiadiazol-2-yl)-6-methylnicotinamide

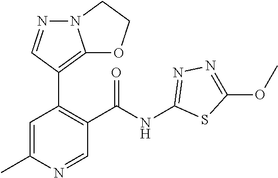

To a stirred solution of 4-(2,3-dihydropyrazolo(5,1-b)oxazol-7-yl)-6-methylnicotinic acid (85.0 mg, 0.35 mmol) in Acetonitrile (4 mL) were added 5-methoxy-1,3,4-thiadiazol-2-amine (68.0 mg, 0.52 mmol) and NMI (285.0 mg, 3.47 mmol). To the above solution was added TCFH (97.0 mg, 0.35 mmol) in MeCN (0.5 mL) at 25° C. under nitrogen atmosphere. The resulting solution was stirred at 25° C. for 1 hr under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (0.5 mL) which was applied to a 20 g C18 column, purified by Combi Flash (Biotage Isolera Prime) and eluted with 5˜80% acetonitrile in water within 30 min to afford 4-(2,3-dihydropyrazolo(5,1-b)oxazol-7-yl)-N-(5-methoxy-1,3,4-thiadiazol-2-yl)-6-methylnicotinamide (64.7 mg, 50%) as a yellow solid. MS (ESI) calc'd for (C₁₅H₁₄N₆O₃S) (M+1)⁺, 359.1. found, 359.1. ¹H NMR (400 MHz, DMSO-d₆) δ 12.82 (s, 1H), 8.49 (s, 1H), 7.57 (s, 1H), 7.38 (s, 1H), 5.17-5.06 (m, 2H), 4.37-4.24 (m, 2H), 4.10 (s, 3H), 2.51 (s, 3H).

### Example 127 and 128

### (R)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide and (S)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide

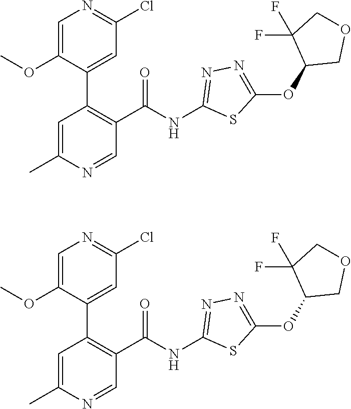 [SMILES: COc1cnc(Cl)cc1-c1cc(C)ncc1C(=O)Nc1nnc(O[C@@H]2COCC2(F)F)s1 ; COc1cnc(Cl)cc1-c1cc(C)ncc1C(=O)Nc1nnc(O[C@H]2COCC2(F)F)s1]

### Step-1: O-(4,4-difluorotetrahydrofuran-3-yl)S-methyl Carbonodithioate

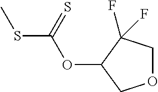

To a solution of NaH (38.7 mg, 0.97 mmol, 60%) in THF (2 mL) was added a solution of 4,4-difluorotetrahydrofuran-3-ol (100.0 mg, 0.81 mmol) in THF (2 mL) dropwise at 0° C. The resulting mixture was stirred at 0° C. for 30 min. To the above mixture was added CS₂ (92.0 mg, 1.21 mmol) dropwise at 0° C. and stirred at 0° C. for 20 min. Then MeI (172.0 mg, 1.21 mmol) was added to the above mixture dropwise at 0° C. The resulting mixture was stirred at 0° C. for 1 h under nitrogen. The resulting mixture was quenched with water. The aqueous layer was extracted with ethyl acetate. The organic layers were combined, washed with brine, dried over anhydrous sodium sulfate and filtered. The filtrate was concentrated under vacuum to afford O-(4,4-difluorotetrahydrofuran-3-yl)S-methyl carbonodithioate (180.0 mg, crude) as yellow oil. MS (ESI) calculated for (C₆H₈F₂O₂S₂) (M+1)⁺, 215.0.

### Step-2: O-(4,4-difluorotetrahydrofuran-3-yl) hydrazinecarbothioate

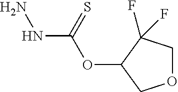 [SMILES: NNC(=S)OC1COCC1(F)F]

To a mixture of O-(4,4-difluorotetrahydrofuran-3-yl)S-methyl carbonodithioate (180.0 mg, 0.84 mmol) in methanol (3 mL) was added hydrazine hydrate (52.56 mg, 0.84 mmol, 80%). The resulting solution was stirred at 23° C. for 1 h. The solvents were removed under vacuum to afford O-(4,4-difluorotetrahydrofuran-3-yl) hydrazinecarbothioate (160.0 mg, crude) as yellow oil. MS (ESI) calculated for (C₅H₈F₂N₂O₂S) (M+1)⁺, 199.0.

### Step-3: 5-((4,4-difluorotetrahydrofuran-3-yl)oxy)-1,3,4-thiadiazol-2-amine

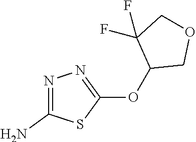 [SMILES: Nc1nnc(OC2COCC2(F)F)s1]

To a mixture of O-(4,4-difluorotetrahydrofuran-3-yl) hydrazinecarbothioate (160.0 mg, 0.81 mmol) in methanol (3 mL) were sequentially added TEA (163.0 mg, 1.62 mmol) and BrCN (94.0 mg, 0.89 mmol). The mixture was stirred at 23° C. for 1 h. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 20 g silica gel column and eluted with 0˜80% ethyl acetate in petroleum ether within 30 min to afford 5-((4,4-difluorotetrahydrofuran-3-yl)oxy)-1,3,4-thiadiazol-2-amine (75.0 mg, 39% over three steps) as a yellow solid. MS (ESI) calculated for (C₆H₇F₂N₃O₂S) (M+1)⁺, 224.0. found, 224.0.

### Step-4: 2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide

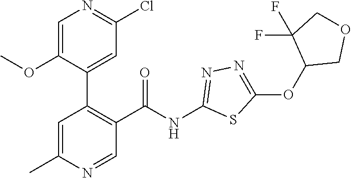 [SMILES: COc1cnc(Cl)cc1-c1cc(C)ncc1C(=O)Nc1nnc(OC2COCC2(F)F)s1]

To a stirred solution of Intermediate G (74.9 mg, 0.27 mmol) in acetonitrile (2 mL) was added 1-methylimidazole (0.1 mL, 1.34 mmol) and 5-((4,4-difluorotetrahydrofuran-3-yl)oxy)-1,3,4-thiadiazol-2-amine (60.0 mg, 0.27 mmol). Then TCFH (75.0 mg, 0.27 mmol) in acetonitrile (0.5 mL) was added to the above mixture at 23° C. The resulting solution was stirred at 23° C. for 2 h under nitrogen. The resulting residue was applied to a 20 g C18 column, purified by Combi Flash (Biotage Isolera Prime) and eluted with 5˜45% acetonitrile in water within 30 min to afford 2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide (60.0 mg, 46%) as a white solid. MS (ESI) calculated for (C₁₉H₁₆ClF₂N₅O₄S) (M+1)⁺, 484.1. found, 484.1.

### Step-5: (R)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide and (S)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide

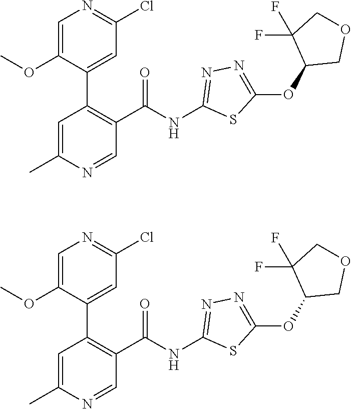 [SMILES: COc1cnc(Cl)cc1-c1cc(C)ncc1C(=O)Nc1nnc(O[C@@H]2COCC2(F)F)s1 ; COc1cnc(Cl)cc1-c1cc(C)ncc1C(=O)Nc1nnc(O[C@H]2COCC2(F)F)s1]

2′-Chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide (60.0 mg) was separated by prep-chiral HPLC with the following condition: (Column: CHIRALPAK IE, 2*25 cm, 5 μm; Mobile Phase A: Hex (0.2% FA)—HPLC, Mobile Phase B: MeOH:EtOH=1:1—HPLC; Flow rate: 15 mL/min; Gradient: 80% B to 80% B in 24 min; Wave Length: 220/254 nm; RT1 (min): 13.10; RT2 (min): 19.39; Sample Solvent: MeOH:DCM=1:1; Injection Volume: 1.5 mL; Number Of Runs: 2) to afford (R)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide (23.5 mg, 39%) as a white solid with the first peak on chiral HPLC and (S)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide (23.2 mg, 38%) as a white solid with the second peak on chiral HPLC. The absolute stereochemistry was not determined.

(R)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide: MS (ESI) calculated for (C₁₉H₁₆ClF₂N₅O₄S) (M+1)⁺, 484.1. found, 484.1. ¹H NMR (400 MHz, DMSO-d₆) δ 13.05 (s, 1H), 8.82 (s, 1H), 8.18 (s, 1H), 7.55 (s, 1H), 7.44 (s, 1H), 5.64-5.60 (m, 1H), 4.44-4.33 (m, 1H), 4.15-3.97 (m, 3H), 3.63 (s, 3H), 2.60 (s, 3H).

(S)-2′-chloro-N-(5-((4,4-difluorotetrahydrofuran-3-yl)oxy)-1,3,4-thiadiazol-2-yl)-5′-methoxy-6-methyl-(4,4′-bipyridine)-3-carboxamide: MS (ESI) calculated for (C₁₉H₁₆ClF₂N₅O₄S) (M+1)⁺, 484.1. found, 484.1. ¹H NMR (400 MHz, DMSO-d₆) δ 13.05 (s, 1H), 8.82 (s, 1H), 8.18 (s, 1H), 7.55 (s, 1H), 7.44 (s, 1H), 5.64-5.60 (m, 1H), 4.39-4.33 (m, 1H), 4.14-3.97 (m, 3H), 3.63 (s, 3H), 2.60 (s, 3H).

### Example 134

### (R)-2′-chloro-6-cyclopentyl-5′-methoxy-N-(5-((tetrahydrofuran-3-yl)oxy)-1,3,4-thiadiazol-2-yl)-(4,4′-bipyridine)-3-carboxamide

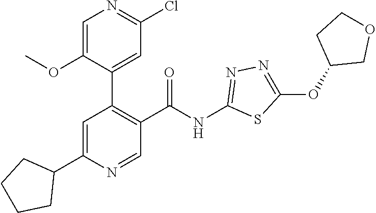 [SMILES: COc1cnc(Cl)cc1-c1cc(C2CCCC2)ncc1C(=O)Nc1nnc(O[C@@H]2CCOC2)s1]

### Step-1: methyl 4-chloro-6-(cyclopent-1-en-1-yl)nicotinate

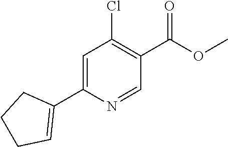 [SMILES: COC(=O)c1cnc(C2=CCCC2)cc1Cl]

To a stirred solution of methyl 6-bromo-4-chloronicotinate (1.0 g, 3.99 mmol) in Water (6 mL) and 1,4-Dioxane (20 mL) were sequentially added 2-(cyclopent-1-en-1-yl)-4,4,5,5-tetramethyl-1,3,2-dioxaborolane (0.6 g, 3.19 mmol), Pd(PPh₃)₂Cl₂ (0.3 g, 0.43 mmol) and K₂CO₃ (1.1 g, 7.96 mmol). The resulting solution was stirred at 60° C. for 1 h under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 40 g silica gel column and eluted with 0˜50% ethyl acetate in petroleum ether within 20 min to afford methyl 4-chloro-6-(cyclopent-1-en-1-yl)nicotinate (360.0 mg, 34%) as a white solid. MS (ESI) calc'd for (C₁₂H₁₂ClNO₂) (M+1)⁺, 238.1. found, 238.1.

### Step-2: methyl 4-chloro-6-cyclopentylnicotinate

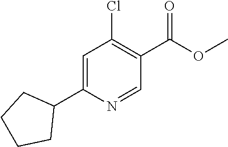 [SMILES: COC(=O)c1cnc(C2CCCC2)cc1Cl]

To a stirred solution of methyl 4-chloro-6-(cyclopent-1-en-1-yl)nicotinate (200.0 mg, 0.72 mmol) in Methanol (5 mL) was added Raney nickel (900.0 mg, 15.33 mmol) at 30° C. under nitrogen atmosphere. The resulting solution was stirred at 30° C. for 5 min under hydrogen (20 atm). The suspension was filtered. The filtrate was collected and concentrated under vacuum. The resulting residue was dissolved in DMF (1 mL) which was applied to a 20 g C18 column, purified by Combi Flash (Biotage Isolera Prime) and eluted with 10˜90% acetonitrile in water within 20 min to afford methyl 4-chloro-6-cyclopentylnicotinate (150.0 mg, 80%) as yellow oil. MS (ESI) calc'd for (C₁₂H₁₄ClNO₂) (M+1)⁺, 240.1. found, 240.1.

### Step-3: methyl 2′-chloro-6-cyclopentyl-5′-methoxy-(4,4′-bipyridine)-3-carboxylate

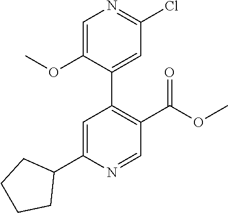 [SMILES: COC(=O)c1cnc(C2CCCC2)cc1-c1cc(Cl)ncc1OC]

To a stirred solution of methyl 4-chloro-6-cyclopentylnicotinate (140.0 mg, 0.58 mmol) in 1,4-Dioxane (2 mL) and Water (0.2 mL) were sequentially added (2-chloro-5-methoxypyridin-4-yl)boronic acid (109.0 mg, 0.58 mmol), Pd(dtbpf)Cl₂ (38.1 mg, 0.06 mmol) and K₂CO₃ (161.0 mg, 1.17 mmol). The resulting solution was stirred at 80° C. for 16 h under nitrogen. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (1 mL) which was applied to a 25 g C18 column and purified by Combi Flash (Biotage Isolera Prime), eluted with 10˜100% acetonitrile in water within 20 min to afford methyl 2′-chloro-6-cyclopentyl-5′-methoxy-(4,4′-bipyridine)-3-carboxylate (120.0 mg, 50%) as yellow oil. MS (ESI) calc'd for (C₁₈H₁₉ClN₂O₃) (M+1)⁺, 347.1. found, 347.1.

### Step-4: 2′-chloro-6-cyclopentyl-5′-methoxy-(4,4′-bipyridine)-3-carboxylic Acid

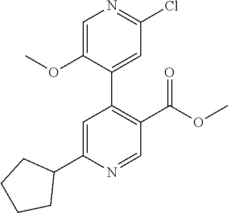 [SMILES: COC(=O)c1cnc(C2CCCC2)cc1-c1cc(Cl)ncc1OC]

To a stirred solution of methyl 2′-chloro-6-cyclopentyl-5′-methoxy-(4,4′-bipyridine)-3-carboxylate (110.0 mg, 0.32 mmol) in Tetrahydrofuran (1 mL) and Water (1 mL) was added lithium hydroxide (38.0 mg, 1.59 mmol). The resulting solution was stirred at room temperature for 2 h. The aqueous layer was acidified with HCl (1 N) to pH ˜3 and extracted with ethyl acetate. The combined organic layers were washed with brine, dried over anhydrous sodium sulfate, filtered, and concentrated under vacuum. The resulting residue was dissolved in DMF (1 mL) which was applied to a 20 g C18 column, purified by Combi Flash (Biotage Isolera Prime) and eluted with 30˜80% acetonitrile in water within 15 min to afford 2′-chloro-6-cyclopentyl-5′-methoxy-(4,4′-bipyridine)-3-carboxylic acid (80.0 mg, 74%) as a white solid. MS (ESI) calc'd for (C₁₇H₁₇ClN₂O₃) (M+1)⁺, 333.1. found, 333.1.

### Step-5: (R)-5-((tetrahydrofuran-3-yl)oxy)-1,3,4-thiadiazol-2-amine

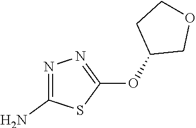 [SMILES: Nc1nnc(O[C@@H]2CCOC2)s1]

To a solution of (R)-tetrahydrofuran-3-ol (1.0 g, 11.35 mmol) in Tetrahydrofuran (10 mL) was added NaH (460 mg, 13.50 mmol, 60%) in portions at 0° C. and stirred at 0° C. for 1 h under nitrogen atmosphere. To the above mixture was added 5-bromo-1,3,4-thiadiazol-2-amine (2.0 g, 11.35 mmol) at 0° C. under nitrogen atmosphere. The resulting solution was stirred at 0° C. for 2 h. The reaction mixture was then quenched by the addition of water and extracted with ethyl acetate. The combined organic layers were washed with brine, dried over anhydrous sodium sulfate, filtered, and concentrated under vacuum. The resulting residue was dissolved in DMF (4 mL) which was applied to a 80 g C18 column and purified by Combi Flash (Biotage Isolera Prime), eluted with 5˜15% acetonitrile in water within 25 mins to afford (R)-5-((tetrahydrofuran-3-yl)oxy)-1,3,4-thiadiazol-2-amine (330 mg, 14%) as a white solid. MS (ESI) calc'd for (C₆H₉N₃O₂S) (M+1)⁺, 188.0. found, 188.1.

### Step-6: (R)-2′-chloro-6-cyclopentyl-5′-methoxy-N-(5-((tetrahydrofuran-3-yl)oxy)-1,3,4-thiadiazol-2-yl)-(4,4′-bipyridine)-3-carboxamide

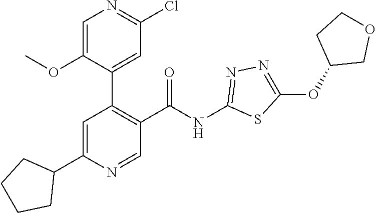 [SMILES: COc1cnc(Cl)cc1-c1cc(C2CCCC2)ncc1C(=O)Nc1nnc(O[C@@H]2CCOC2)s1]

To a stirred solution of 2′-chloro-6-cyclopentyl-5′-methoxy-(4,4′-bipyridine)-3-carboxylic acid (30.0 mg, 0.09 mmol) in acetonitrile (1 mL) were added (R)-5-((tetrahydrofuran-3-yl)oxy)-1,3,4-thiadiazol-2-amine (16.9 mg, 0.09 mmol) and 1-methyl-1H-imidazole (37.0 mg, 0.45 mmol). To the above solution was added TCFH (25.3 mg, 0.09 mmol) in acetonitrile (1 mL). The resulting solution was stirred at 25° C. for 1 h under nitrogen. The organic solvent was removed under vacuum. The resulting residue was dissolved in DMF (1 mL) which was applied to a 20 g C18 column, purified by Combi Flash (Biotage Isolera Prime) and eluted with 40˜70% acetonitrile in water within 35 min to afford (R)-2′-chloro-6-cyclopentyl-5′-methoxy-N-(5-((tetrahydrofuran-3-yl)oxy)-1,3,4-thiadiazol-2-yl)-(4,4′-bipyridine)-3-carboxamide (30.3 mg, 66%) as a white solid. MS (ESI) calc'd for (C₂₃H₂₄ClN₅O₄S) (M+1)⁺, 502.1. found, 502.1. ¹H NMR (400 MHz, DMSO-d₆) δ 12.86 (s, 1H), 8.83 (s, 1H), 8.17 (s, 1H), 7.58 (s, 1H), 7.43 (s, 1H), 5.55-5.48 (m, 1H), 4.02-3.76 (m, 4H), 3.63 (s, 3H), 3.22-3.20 (m, 1H), 2.32-2.22 (m, 1H), 2.18-2.00 (m, 3H), 1.81-1.68 (m, 6H).

### Example 136 and 137

### (S)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide and (R)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide

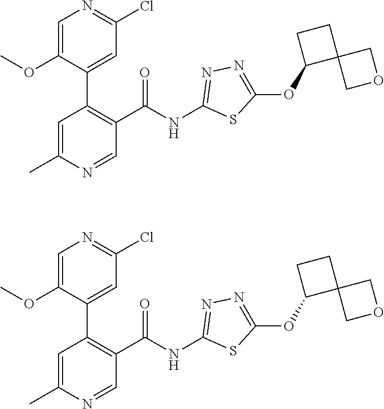 [SMILES: COc1cnc(Cl)cc1-c1cc(C)ncc1C(=O)Nc1nnc(O[C@H]2CCC23COC3)s1 ; COc1cnc(Cl)cc1-c1cc(C)ncc1C(=O)Nc1nnc(O[C@@H]2CCC23COC3)s1]

### Step-1: S-methyl O-(2-oxaspiro(3.3)heptan-5-yl) carbonodithioate

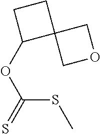

To a stirred solution of 2-oxaspiro(3.3)heptan-5-ol (500.0 mg, 4.38 mmol) in THF (10 mL) was added NaH (350.0 mg, 8.75 mmol, 60%) in portions at 0° C. The resulting solution was stirred at 0° C. for 0.5 h under nitrogen atmosphere. To the above solution was added CS₂ (500.0 mg, 6.57 mmol) at 0° C. The resulting mixture was then stirred at 0° C. for 0.5 h. To the above solution was added MeI (935.0 mg, 6.59 mmol) at 0° C. The resulting mixture was then stirred at 0° C. for 0.5 h under nitrogen atmosphere. The reaction mixture was quenched by the addition of water and extracted with ethyl acetate. The combined organic layers were washed with brine, dried over anhydrous sodium sulfate, filtered, and concentrated under vacuum to afford S-methyl O-(2-oxaspiro(3.3)heptan-5-yl) carbonodithioate (620.0 mg, crude) as yellow oil, the crude product was used in the next step without further purification.

### Step-2: O-(2-oxaspiro(3.3)heptan-5-yl) hydrazinecarbothioate

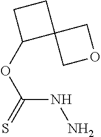 [SMILES: NNC(=S)OC1CCC12COC2]

To a stirred solution of S-methyl O-(2-oxaspiro(3.3)heptan-5-yl) carbonodithioate (620.0 mg, 3.03 mmol) in Methanol (8 mL) was added hydrazine hydrate (190.0 mg, 3.03 mmol, 80%) at 25° C. The resulting solution was stirred at 25° C. for 0.5 h under nitrogen atmosphere. The solvents were removed under vacuum to afford O-(2-oxaspiro(3.3)heptan-5-yl) hydrazinecarbothioate (450.0 mg, crude) as a yellow oil, which was used in the next step without further purification.

### Step-3: 5-((2-oxaspiro(3.3)heptan-5-yl)oxy)-1,3,4-thiadiazol-2-amine

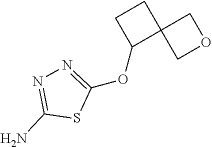 [SMILES: Nc1nnc(OC2CCC23COC3)s1]

To a stirred solution of O-(2-oxaspiro(3.3)heptan-5-yl) hydrazinecarbothioate (450.0 mg, 2.39 mmol) in Methanol (8 mL) was added BrCN (279.0 mg, 2.63 mmol) and TEA (483.0 mg, 4.78 mmol) at 25° C. The resulting solution was stirred at 25° C. for 1 h under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (1 mL) and purified by Combi Flash (Biotage Isolera Prime) which applied to a 40 g silica gel column that was eluted with 0˜10% methanol in dichloromethane within 35 min to afford 5-((2-oxaspiro(3.3)heptan-5-yl)oxy)-1,3,4-thiadiazol-2-amine (280.0 mg, 51%) as a yellow solid. MS (ESI) calc'd for (C₈H₁₁N₃O₂S) (M+1)⁺, 214.1. found, 214.1.

### Step-4: N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide

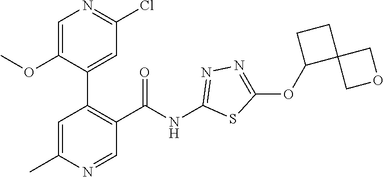 [SMILES: COc1cnc(Cl)cc1-c1cc(C)ncc1C(=O)Nc1nnc(OC2CCC23COC3)s1]

To a stirred solution of 5-((2-oxaspiro(3.3)heptan-5-yl)oxy)-1,3,4-thiadiazol-2-amine (280.0 mg, 1.22 mmol) in Acetonitrile (5 mL) was added Intermediate G (340.0 mg, 1.22 mmol) and NMI (501.0 mg, 6.11 mmol). To the above solution was added TCFH (343.0 mg, 1.22 mmol) in MeCN (0.5 mL) at 25° C. The resulting solution was stirred at 25° C. for 1 h under nitrogen atmosphere. The solvents were removed under vacuum. The resulting residue was dissolved in DMF (1 mL) which was applied to a 25 g C18 column, purified by Combi Flash (Biotage Isolera Prime) and eluted with 5˜80% acetonitrile in water within 30 min to afford N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide (170.0 mg, 27%) as a white solid. MS (ESI) calc'd for (C₂₁H₂₀ClN₅O₄S) (M+1)⁺, 474.1. found, 474.1.

### Step-5: (S)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide and (R)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide

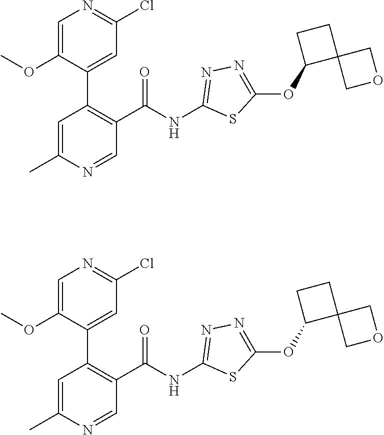 [SMILES: COc1cnc(Cl)cc1-c1cc(C)ncc1C(=O)Nc1nnc(O[C@H]2CCC23COC3)s1 ; COc1cnc(Cl)cc1-c1cc(C)ncc1C(=O)Nc1nnc(O[C@@H]2CCC23COC3)s1]

N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide (170.0 mg) was separated by prep-chiral HPLC with the following condition: (Column: CHIRALPAK IE, 2*25 cm, 5 um; Mobile Phase A: MtBE (0.1% FA)—HPLC, Mobile Phase B: MeOH:DCM=1:1; Flow rate: 18 mL/min; Gradient: 50% B to 50% B in 20 min; Wave Length: 220/254 nm; RT1 (min): 12.21; RT2 (min): 15.93; Sample Solvent: MeOH:DCM=1:1; Injection Volume: 0.8 mL; Number Of Runs: 4) to afford (S)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide (40.9 mg, 24%) as a white solid with the first peak on chiral HPLC and (R)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide (34.0 mg, 19%) as a white solid with the second peak on chiral HPLC. The absolute stereochemistry was not determined.

(S)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide: MS (ESI) calc'd for (C₂₁H₂₀ClN₅O₄S) (M+1)⁺, 474.1. found, 474.1. ¹H NMR (400 MHz, DMSO-d₆) δ 12.92 (s, 1H), 8.82 (s, 1H), 8.18 (s, 1H), 7.54 (s, 1H), 7.44 (s, 1H), 5.24-5.15 (m, 1H), 4.87 (d, J=6.4 Hz, 1H), 4.76 (d, J=6.8 Hz, 1H), 4.53 (d, J=6.4 Hz, 1H), 4.46 (d, J=6.8 Hz, 1H), 3.65 (s, 3H), 2.60 (s, 3H), 2.32-2.21 (m, 1H), 2.19-2.06 (m, 1H), 1.94-1.79 (m, 2H).

(R)—N-(5-((2-oxaspiro(3.3)heptan-5-yl)oxy)-1,3,4-thiadiazol-2-yl)-2′-chloro-5′-methoxy-6-methyl-(4,4′-bipyridine)-3-carboxamide: MS (ESI) calc'd for (C₂₁H₂₂ClN₅O₄S) (M+1)⁺, 474.1. found, 474.2. ¹H NMR (400 MHz, DMSO-d₆) δ 12.90 (s, 1H), 8.82 (s, 1H), 8.18 (s, 1H), 7.54 (s, 1H), 7.43 (s, 1H), 5.23-5.15 (m, 1H), 4.87 (d, J=6.4 Hz, 1H), 4.76 (d, J=6.8 Hz, 1H), 4.53 (d, J=6.4 Hz, 1H), 4.46 (d, J=6.8 Hz, 1H), 3.65 (s, 3H), 2.60 (s, 3H), 2.32-2.21 (m, 1H), 2.19-2.06 (m, 1H), 1.94-1.82 (m, 2H).

### Example 144

### 2′-chloro-5′-methoxy-N-(5-methoxy-1,3,4-thiadiazol-2-yl)-6-((3-methoxyazetidin-1-yl)methyl)-(4,4′-bipyridine)-3-carboxamide

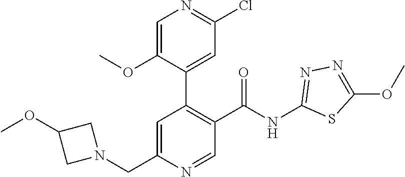

### Step-1: ethyl 4-chloro-6-((3-methoxyazetidin-1-yl)methyl)nicotinate

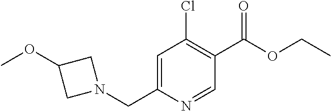 [SMILES: CCOC(=O)c1cnc(CN2CC(OC)C2)cc1Cl]

To a degassed solution of ethyl 4-chloro-6-(chloromethyl)nicotinate (500.0 mg, 2.14 mmol) in dry Ethanol (5 mL) were added 3-methoxyazetidine (149.0 mg, 1.71 mmol) and TEA (432.0 mg, 4.27 mmol) dropwise at 25° C. and stirred at 25° C. for 1 hr under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (2 mL) and purified by Combi Flash which applied to a 40 g silica gel column that was eluted with 0˜50% ethyl acetate in petroleum ether within 25 min to afford ethyl 4-chloro-6-((3-methoxyazetidin-1-yl)methyl)nicotinate (460.0 mg, 56%) as yellow oil. MS (ESI) calculated for (C₁₃H₁₇ClN₂O₃) (M+1)⁺, 285.1. found, 285.1.

### Step-2: ethyl 2′-chloro-5′-methoxy-6-((3-methoxyazetidin-1-yl)methyl)-(4,4′-bipyridine)-3-carboxylate

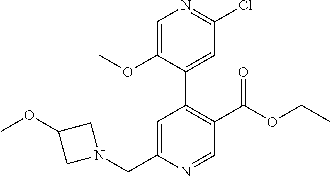 [SMILES: CCOC(=O)c1cnc(CN2CC(OC)C2)cc1-c1cc(Cl)ncc1OC]

To a stirred solution of ethyl 4-chloro-6-((3-methoxyazetidin-1-yl)methyl)nicotinate (460.0 mg, 1.62 mmol) in dioxane (5 ml) were added (2-chloro-5-methoxypyridin-4-yl)boronic acid (908.0 mg, 4.85 mmol), K₂CO₃ (447.0 mg, 3.23 mmol) and Pd(dppf)Cl₂ (119.0 mg, 0.16 mmol) at 25° C. The reaction mixture was stirred at 80° C. for 2 h under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash which applied to 40 g silica gel column and eluted with 0˜50% ethyl acetate in petroleum ether within 25 min to afford ethyl 2′-chloro-5′-methoxy-6-((3-methoxyazetidin-1-yl)methyl)-(4,4′-bipyridine)-3-carboxylate (250.0 mg, 23%) as a white solid. MS (ESI) calculated for (C₁₉H₂₂ClN₃O₄) (M+1)⁺, 392.1. found, 392.1.

### Step-3: 2′-chloro-5′-methoxy-6-((3-methoxyazetidin-1-yl)methyl)-(4,4′-bipyridine)-3-carboxylic Acid

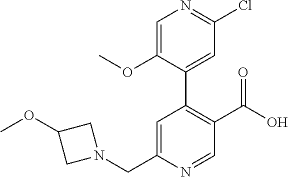 [SMILES: COc1cnc(Cl)cc1-c1cc(CN2CC(OC)C2)ncc1C(=O)O]

To a stirred solution of ethyl 2′-chloro-5′-methoxy-6-((3-methoxyazetidin-1-yl)methyl)-(4,4′-bipyridine)-3-carboxylate (250.0 mg, 0.64 mmol) in Tetrahydrofuran (THF) (2 mL) and Water (2 mL) was added LiOH (134.0 mg, 3.19 mmol) at 25° C. The resulting solution was stirred at 25° C. for 2 h. The organic solvent was removed under vacuum. The aqueous layer was acidified with Citric acid to pH ˜2 and extracted with ethyl acetate. The combined organic layers were washed with brine, dried over anhydrous sodium sulfate, filtered, and concentrated under vacuum. The resulting residue was dissolved in DMF (1 mL) which was applied to a 25 g C18 column and purified by Combi Flash, eluted with 5˜30% acetonitrile in water within 20 min to afford 2′-chloro-5′-methoxy-6-((3-methoxyazetidin-1-yl)methyl)-(4,4′-bipyridine)-3-carboxylic acid (90.0 mg, 36%) as a white solid. MS (ESI) calculated for (C₁₇H₁₈ClN₃O₄) (M+1)⁺, 364.1. found, 364.1.

### Step-4: 2′-chloro-5′-methoxy-N-(5-methoxy-1,3,4-thiadiazol-2-yl)-6-((3-methoxyazetidin-1-yl)methyl)-(4,4′-bipyridine)-3-carboxamide

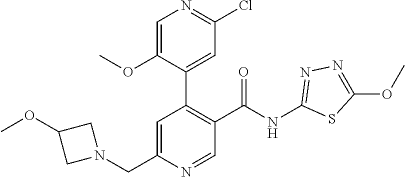

To a stirred solution of 2′-chloro-5′-methoxy-6-((3-methoxyazetidin-1-yl)methyl)-(4,4′-bipyridine)-3-carboxylic acid (100.0 mg, 0.28 mmol) in acetonitrile (2 mL) were added 5-methoxy-1,3,4-thiadiazol-2-amine (36.1 mg, 0.28 mmol) and NMI (113.0 mg, 1.37 mmol). To the above solution was added TCFH (77.0 mg, 0.28 mmol) in acetonitrile (1 mL). The resulting solution was stirred at 25° C. under nitrogen for 1 h. The solvents were removed under vacuum. The resulting residue was dissolved in DMF (2 mL) and was purified by prep-HPLC with the following condition: (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 30% B in 10 min, 30% B; Wave Length: 220 nm; RT1 (min): 8.25; Injection Volume: 1.2 mL; Number Of Runs: 3) to afford 2′-chloro-5′-methoxy-N-(5-methoxy-1,3,4-thiadiazol-2-yl)-6-((3-methoxyazetidin-1-yl)methyl)-(4,4′-bipyridine)-3-carboxamide (15.1 mg, 11%) as a white solid. MS (ESI) calculated for (C₂₀H₂₁ClN₆O₄S) (M+1)⁺, 477.1. found, 477.2. ¹H NMR (400 MHz, DMSO-d₆) δ 12.73 (s, 1H), 8.27 (s, 1H), 8.12 (s, 1H), 7.20 (s, 1H), 6.40 (s, 1H), 4.32-4.22 (m, 1H), 4.18-4.06 (m, 5H), 3.98-3.92 (m, 2H), 3.90 (s, 3H), 3.74-3.68 (m, 2H), 3.21 (s, 3H).

### Example 158

### 2′-chloro-5′-methoxy-N-(5-methoxy-1,3,4-thiadiazol-2-yl)-6-(methoxymethyl)-(4,4′-bipyridine)-3-carboxamide

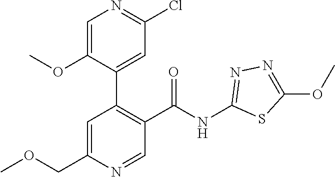

### Step-1: ethyl 4-chloro-6-vinylnicotinate

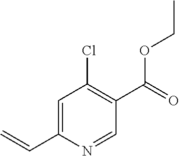

To a stirred solution of ethyl 4,6-dichloronicotinate (10.0 g, 45.40 mmol) in 1,4-Dioxane (60 mL) and water (6 mL) were added trifluoro(vinyl)-14-borane, potassium salt (6.1 g, 45.40 mmol), K₂CO₃ (12.5 g, 91.00 mmol) and PdCl2(dppf) CH₂Cl₂ (3.7 g, 4.54 mmol) at 23° C. The resulting solution was stirred at 80° C. for 1.5 hr under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 120 g silica gel column and eluted with 0˜50% ethyl acetate in petroleum ether within 35 min to afford ethyl 4-chloro-6-vinylnicotinate (9.0 g, 75%) as a yellow solid. MS (ESI) calc'd for (C₁₀H₁₀ClNO₂) (M+1)⁺, 212.0. found, 212.0.

### Step-2: ethyl 4-chloro-6-formylnicotinate

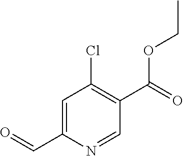

To a stirred solution of ethyl 4-chloro-6-vinylnicotinate (9.0 g, 42.50 mmol) in Tetrahydrofuran (210 mL) and Water (70 mL) were added osmium tetroxide (2.1 g, 8.50 mmol) and sodium periodate (18.2 g, 85.00 mmol) at 20° C. The resulting solution was stirred at 20° C. for 16 h. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (8 mL) and purified by Combi Flash (Biotage Isolera Prime) which applied to a 330 g silica gel column that was eluted with 0˜50% ethyl acetate in petroleum ether within 25 min to afford ethyl 4-chloro-6-formylnicotinate (3.1 g, 31%) as a yellow solid. MS (ESI) calc'd for (C₉H₈ClNO₃) (M+1)⁺, 214.0. found, 214.0.

### Step-3: ethyl 4-chloro-6-(hydroxymethyl)nicotinate

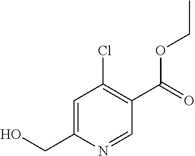

To a stirred solution of ethyl 4-chloro-6-formylnicotinate (2.0 g, 9.36 mmol) in Ethanol (20 mL) was added NaBH₄ (0.4 g, 9.34 mmol) at 0° C. The resulting solution was stirred at 0° C. for 30 min. The reaction mixture was quenched by the addition of water and extracted with ethyl acetate. The combined organic layers were washed with brine, dried over anhydrous sodium sulfate, filtered, and concentrated under vacuum to afford ethyl 4-chloro-6-(hydroxymethyl)nicotinate (2.0 g, crude) as a yellow oil. MS (ESI) calc'd for (C₉H₁₀ClNO₃) (M+1)⁺, 216.0. found, 216.0.

### Step-4: ethyl 2′-chloro-6-(hydroxymethyl)-5′-methoxy-(4,4′-bipyridine)-3-carboxylate

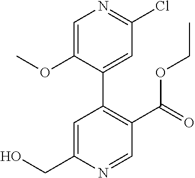 [SMILES: CCOC(=O)c1cnc(CO)cc1-c1cc(Cl)ncc1OC]

To a stirred solution of ethyl 4-chloro-6-(hydroxymethyl)nicotinate (2.0 g, 9.28 mmol) in 1,4-Dioxane (20 mL) were added (2-chloro-5-methoxypyridin-4-yl)boronic acid (2.4 g, 12.99 mmol), K₂CO₃ (3.9 g, 27.80 mmol) in Water (4 mL) and Pd(dtbpf)Cl₂ (604.0 mg, 0.93 mmol) at 20° C. The resulting solution was stirred at 80° C. for 1 h under nitrogen atmosphere. The reaction mixture was diluted with ethyl acetate and filtered. The filtrate was collected and washed with brine, dried over anhydrous sodium sulfate, filtered, and concentrated under vacuum. The resulting residue was dissolved in DCM (8 mL) and purified by Combi Flash (Biotage Isolera Prime) which applied to an 80 g silica gel column that was eluted with 0˜90% ethyl acetate in petroleum ether within 30 min to afford ethyl 2′-chloro-6-(hydroxymethyl)-5′-methoxy-(4,4′-bipyridine)-3-carboxylate (1.1 g, 30%) as a brown oil. MS (ESI) calculated for (C₁₅H₁₅ClN₂O₄) (M+1)⁺, 323.1. found, 323.1.

### Step-5: ethyl 2′-chloro-5′-methoxy-6-(methoxymethyl)-(4,4′-bipyridine)-3-carboxylate

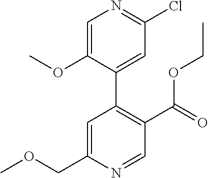 [SMILES: CCOC(=O)c1cnc(COC)cc1-c1cc(Cl)ncc1OC]

To a stirred solution of NaH (109.1 mg, 2.73 mmol, 60%) in N,N-Dimethylformamide (2 ml) were added a solution of ethyl 2′-chloro-6-(hydroxymethyl)-5′-methoxy-(4,4′-bipyridine)-3-carboxylate (600.0 mg, 1.86 mmol) in N,N-Dimethylformamide (2 ml) at 0° C. The resulting solution was stirred at 0° C. for 30 min. To the above solution was added dimethyl sulfate (251.0 mg, 1.99 mmol) at 0° C. under nitrogen. The resulting mixture was then stirred at 25° C. for 3 hr. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (2 mL) which was applied to a 40 g C18 column and purified by Combi Flash (Biotage Isolera Prime), eluted with 5˜30% acetonitrile in water within 30 min to afford ethyl 2′-chloro-5′-methoxy-6-(methoxymethyl)-(4,4′-bipyridine)-3-carboxylate (210.0 mg, 34%) as a brown oil. MS (ESI) calculated for (C₁₆H₁₇ClN₂O₄) (M+1)⁺, 337.1. found, 337.1.

### Step-6: 2′-chloro-5′-methoxy-6-(methoxymethyl)-(4,4′-bipyridine)-3-carboxylic Acid

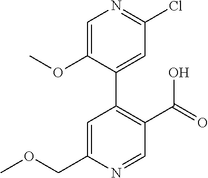 [SMILES: COCc1cc(-c2cc(Cl)ncc2OC)c(C(=O)O)cn1]

To a stirred solution of ethyl 2′-chloro-5′-methoxy-6-(methoxymethyl)-(4,4′-bipyridine)-3-carboxylate (180.0 mg, 0.53 mmol) in Tetrahydrofuran (THF) (1 mL) was added LiOH (38.4 mg, 1.60 mmol) in Water (0.3 mL) at 25° C. The resulting solution was stirred at 25° C. for 16 h. The reaction mixture was acidified with citric acid to pH 4˜5 and purified directly. The resulting residue was diluted with acetonitrile (2 mL) which was applied to a 40 g C18 column, purified by Combi Flash (Biotage Isolera Prime) and eluted with 5˜30% acetonitrile in water (0.05% FA) within 30 min to afford 2′-chloro-5′-methoxy-6-(methoxymethyl)-(4,4′-bipyridine)-3-carboxylic acid (120.0 mg, 70%) as a white solid. MS (ESI) calculated for (C₁₄H₁₃ClN₂O₄) (M+1)⁺, 309.1. found, 309.1.

### Step-7: 2′-chloro-5′-methoxy-N-(5-methoxy-1,3,4-thiadiazol-2-yl)-6-(methoxymethyl)-(4,4′-bipyridine)-3-carboxamide

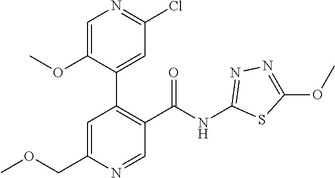

To a stirred solution of 2′-chloro-5′-methoxy-6-(methoxymethyl)-(4,4′-bipyridine)-3-carboxylic acid (60.0 mg, 0.19 mmol) in Acetonitrile (1 mL) were added 1-methyl-1H-imidazole (80.0 mg, 0.97 mmol) and 5-methoxy-1,3,4-thiadiazol-2-amine (25.5 mg, 0.19 mmol) at 25° C. To the above solution was added TCFH (54.5 mg, 0.19 mmol) in MeCN (0.5 mL) at 25° C. under nitrogen. The resulting mixture was then stirred at 25° C. for 2 hr. The residue was dissolved in DMF (1 mL) and purified by prep-HPLC with the following condition: (Column: XBridge Prep OBD C18 Column, 30*150 mm, 5 um; Mobile Phase A: Water (10 mmol/L NH₄HCO₃), Mobile Phase B: ACN; Flow rate: 60 mL/min; Gradient: 16% B to 26% B in 8 min, 26% B; Wave Length: 254 nm; RT1 (min): 7) to afford 2′-chloro-5′-methoxy-N-(5-methoxy-1,3,4-thiadiazol-2-yl)-6-(methoxymethyl)-(4,4′-bipyridine)-3-carboxamide (19.1 mg, 23%) as a white solid. MS (ESI) calculated for (C₁₇H₁₆ClN₅O₄S) (M+1)⁺, 422.1. found, 422.1. ¹H NMR (400 MHz, DMSO-d₆) δ 12.97 (s, 1H), 8.91 (s, 1H), 8.18 (s, 1H), 7.54 (s, 1H), 7.46 (s, 1H), 4.62 (s, 2H), 4.07 (s, 3H), 3.64 (s, 3H), 3.42 (s, 3H).

### Example 173 and 174

### N-(5-(((5s,8r)-1-oxaspiro(4.5)decan-8-yl)oxy)-1,3,4-thiadiazol-2-yl)-2′-chloro-5′-methoxy-6-methyl-(4,4′-bipyridine)-3-carboxamide and N-(5-(((5r,8s)-1-oxaspiro(4.5)decan-8-yl)oxy)-1,3,4-thiadiazol-2-yl)-2′-chloro-5′-methoxy-6-methyl-(4,4′-bipyridine)-3-carboxamide

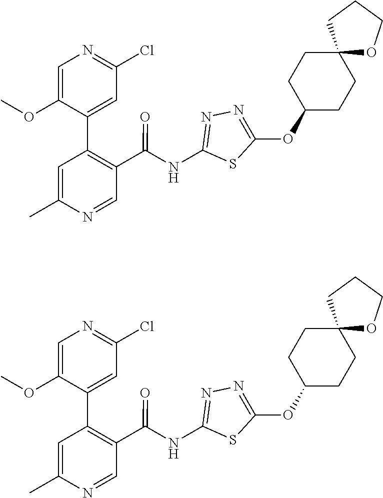 [SMILES: COc1cnc(Cl)cc1-c1cc(C)ncc1C(=O)Nc1nnc(O[C@H]2CC[C@]3(CCCO3)CC2)s1 ; COc1cnc(Cl)cc1-c1cc(C)ncc1C(=O)Nc1nnc(O[C@H]2CC[C@@]3(CCCO3)CC2)s1]

### Step-1: 1-oxaspiro(4.5)decan-8-ol

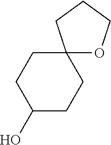 [SMILES: OC1CCC2(CCCO2)CC1]

To a stirred solution of 1-oxaspiro(4.5)decan-8-one (100.0 mg, 0.64 mmol) in Methanol (1 mL) was added NaBH₄ (50.0 mg, 1.32 mmol) in portions at 0° C. under nitrogen atmosphere. The resulting solution was stirred at 25° C. for 2 h under nitrogen atmosphere. The reaction mixture was quenched by the addition of water and extracted with ethyl acetate. The combined organic layers were washed with brine, dried over anhydrous sodium sulfate, filtered, and concentrated under vacuum. The resulting residue was dissolved in DCM (1 mL) and purified by Combi Flash (Biotage Isolera Prime) which applied to a 20 g silica gel column that was eluted with 0˜50% ethyl acetate in petroleum ether within 35 min to afford 1-oxaspiro(4.5)decan-8-ol (80.0 mg, 71%) as colorless oil.

### Step-2: S-methyl O-(1-oxaspiro(4.5)decan-8-yl) carbonodithioate

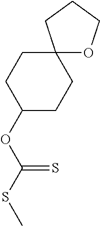

To a mixture of NaH (41.0 mg, 1.02 mmol, 60%) in THF (5 mL) was added 1-oxaspiro(4.5)decan-8-ol (80.0 mg, 0.51 mmol) in portions at 0° C. and stirred at 0° C. for 1 h under nitrogen. Then CS₂ (58.0 mg, 0.76 mmol) was added to the above mixture and stirred at 0° C. for 20 min, and then MeI (109.0 mg, 0.76 mmol) was added to the above mixture at 5° C. The resulting mixture was stirred at 0° C. for 1 h. The reaction mixture was quenched by the addition of water and extracted with ethyl acetate. The combined organic layers were washed with brine, dried over anhydrous sodium sulfate, filtered, and concentrated under vacuum to afford S-methyl O-(1-oxaspiro(4.5)decan-8-yl) carbonodithioate (100.0 mg, crude) as yellow oil, which was used in the next step without further purification.

### Step-3: O-(1-oxaspiro(4.5)decan-8-yl) hydrazinecarbothioate

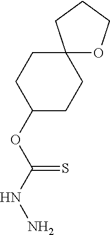 [SMILES: NNC(=S)OC1CCC2(CCCO2)CC1]

To a stirred solution of S-methyl O-(1-oxaspiro(4.5)decan-8-yl) carbonodithioate (100.0 mg, 0.40 mmol) in Methanol (4 mL) was added hydrazine hydrate (20.0 mg, 0.49 mmol, 80%) at 25° C. under nitrogen atmosphere. The resulting solution was stirred at 25° C. for 0.5 h. The solvents were removed under vacuum to afford O-(1-oxaspiro(4.5)decan-8-yl) hydrazinecarbothioate (120.0 mg, crude) as yellow oil, which was used in the next step without further purification.

### Step-4: 5-((1-oxaspiro(4.5)decan-8-yl)oxy)-1,3,4-thiadiazol-2-amine

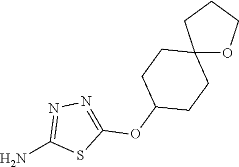 [SMILES: Nc1nnc(OC2CCC3(CCCO3)CC2)s1]

To a stirred solution of O-(1-oxaspiro(4.5)decan-8-yl) hydrazinecarbothioate (120.0 mg, 0.38 mmol) and TEA (76.0 mg, 0.76 mmol) in Methanol (4 mL) was added BrCN (45.0 mg, 0.42 mmol) at 25° C. under nitrogen atmosphere. The resulting solution was stirred at 25° C. for 1 h under nitrogen atmosphere. The solvents were removed under vacuum. The resulting residue was dissolved in DCM (1 mL) and purified by Combi Flash (Biotage Isolera Prime) which applied to a 20 g silica gel column that was eluted with 0˜10% methanol in dichloromethane within 30 min to afford 5-((1-oxaspiro(4.5)decan-8-yl)oxy)-1,3,4-thiadiazol-2-amine (80.0 mg, 81%) as a yellow solid. MS (ESI) calc'd for (C₁₁H₁₇N₃O₂S) (M+1)⁺, 256.1. found, 256.1.

### Step-5: N-(5-((1-oxaspiro(4.5)decan-8-yl)oxy)-1,3,4-thiadiazol-2-yl)-2′-chloro-5′-methoxy-6-methyl-(4,4′-bipyridine)-3-carboxamide

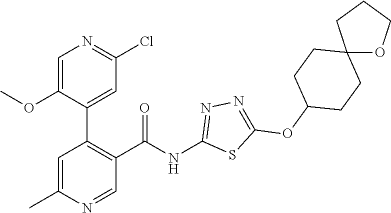 [SMILES: COc1cnc(Cl)cc1-c1cc(C)ncc1C(=O)Nc1nnc(OC2CCC3(CCCO3)CC2)s1]

To a stirred solution of 5-((1-oxaspiro(4.5)decan-8-yl)oxy)-1,3,4-thiadiazol-2-amine (80.0 mg. 0.31 mmol) in acetonitrile (1 mL) were added Intermediate G (87.0 mg, 0.31 mmol) and 1-methyl-1H-imidazole (129.0 mg, 1.56 mmol). To the above solution was added TCFH (88.0 mg, 0.31 mmol) in MeCN (0.5 mL). The resulting solution was stirred at 25° C. under nitrogen for 1 hr. The resulting residue was dissolved in DMF (1 mL) which was applied to a 20 g C18 column and purified by Combi Flash, eluted with 5˜80% acetonitrile in water within 25 min to afford N-(5-((1-oxaspiro(4.5)decan-8-yl)oxy)-1,3,4-thiadiazol-2-yl)-2′-chloro-5′-methoxy-6-methyl-(4,4′-bipyridine)-3-carboxamide (97.0 mg, 42%) as a white solid. MS (ESI) calc'd for (C₂₄H₂₆ClN₅O₄S) (M+1)⁺, 516.1. found, 516.2.

### Step-6: N-(5-(((5s,8r)-1-oxaspiro(4.5)decan-8-yl)oxy)-1,3,4-thiadiazol-2-yl)-2′-chloro-5′-methoxy-6-methyl-(4,4′-bipyridine)-3-carboxamide and N-(5-(((5r,8s)-1-oxaspiro(4.5)decan-8-yl)oxy)-1,3,4-thiadiazol-2-yl)-2′-chloro-5′-methoxy-6-methyl-(4,4′-bipyridine)-3-carboxamide

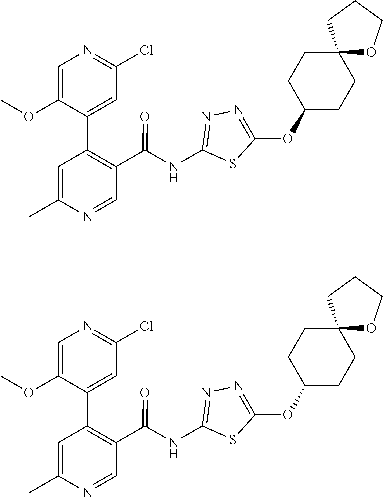 [SMILES: COc1cnc(Cl)cc1-c1cc(C)ncc1C(=O)Nc1nnc(O[C@H]2CC[C@]3(CCCO3)CC2)s1 ; COc1cnc(Cl)cc1-c1cc(C)ncc1C(=O)Nc1nnc(O[C@H]2CC[C@@]3(CCCO3)CC2)s1]

N-(5-((1-Oxaspiro(4.5)decan-8-yl)oxy)-1,3,4-thiadiazol-2-yl)-2′-chloro-5′-methoxy-6-methyl-(4,4′-bipyridine)-3-carboxamide (90.0 mg) was separated by prep-chiral HPLC with the following condition: (Column: CHIRAL ART Amylose-SA, 2*25 cm, 5 μm; Mobile Phase A: Hex (0.2% FA)—HPLC, Mobile Phase B: MeOH:DCM=1:1—HPLC; Flow rate: 20 mL/min; Gradient: 20% B to 20% B in 12.5 min; Wave Length: 220/254 nm; RT1 (min): 9.02; RT2 (min): 11.22; Sample Solvent: MeOH:DCM=1:1—HPLC; Injection Volume: 0.5 mL; Number Of Runs: 5) to afford N-(5-(((5s,8r)-1-oxaspiro(4.5)decan-8-yl)oxy)-1,3,4-thiadiazol-2-yl)-2′-chloro-5′-methoxy-6-methyl-(4,4′-bipyridine)-3-carboxamide (42.6 mg, 47%) as a white solid with the first peak on chiral HPLC and N-(5-(((5r,8s)-1-oxaspiro(4.5)decan-8-yl)oxy)-1,3,4-thiadiazol-2-yl)-2′-chloro-5′-methoxy-6-methyl-(4,4′-bipyridine)-3-carboxamide (22.7 mg, 25%) as a white solid with the second peak on chiral HPLC. The relative stereochemistry was not determined.

N-(5-(((5s,8r)-1-oxaspiro(4.5)decan-8-yl)oxy)-1,3,4-thiadiazol-2-yl)-2′-chloro-5′-methoxy-6-methyl-(4,4′-bipyridine)-3-carboxamide: MS (ESI) calc'd for (C₂₄H₂₆ClN₅O₄S) (M+1)⁺, 516.1. found, 516.2. ¹H NMR (400 MHz, DMSO-d₆) δ 12.85 (s, 1H), 8.81 (s, 1H), 8.18 (s, 1H), 7.54 (s, 1H), 7.43 (s, 1H), 4.95-4.85 (m, 1H), 3.80-3.56 (m, 5H), 2.60 (s, 3H), 2.13-1.75 (m, 6H), 1.74-1.58 (m, 4H), 1.56-1.40 (m, 2H).

N-(5-(((5r,8s)-1-oxaspiro(4.5)decan-8-yl)oxy)-1,3,4-thiadiazol-2-yl)-2′-chloro-5′-methoxy-6-methyl-(4,4′-bipyridine)-3-carboxamide: MS (ESI) calc'd for (C₂₄H₂₆ClN₅O₄S) (M+1)⁺, 516.1. found, 516.2. ¹H NMR (400 MHz, DMSO-d₆) δ 12.86 (s, 1H), 8.80 (s, 1H), 8.18 (s, 1H), 7.53 (s, 1H), 7.43 (s, 1H), 5.04-4.98 (m, 1H), 3.76-3.68 (m, 2H), 3.64 (s, 3H), 2.59 (s, 3H), 2.04-1.94 (m, 2H), 1.94-1.81 (m, 2H), 1.81-1.73 (m, 2H), 1.72-1.58 (m, 4H), 1.55-1.45 (m, 2H).

### Example 185 and 186

### N-(5-(((4s, 7s)-1-oxaspiro(3.5)nonan-7-yl)oxy)-1,3,4-thiadiazol-2-yl)-2′-chloro-5′-methoxy-6-methyl-(4,4′-bipyridine)-3-carboxamide and N-(5-(((4r,7r)-1-oxaspiro(3.5)nonan-7-yl)oxy)-1,3,4-thiadiazol-2-yl)-2′-chloro-5′-methoxy-6-methyl-(4,4′-bipyridine)-3-carboxamide

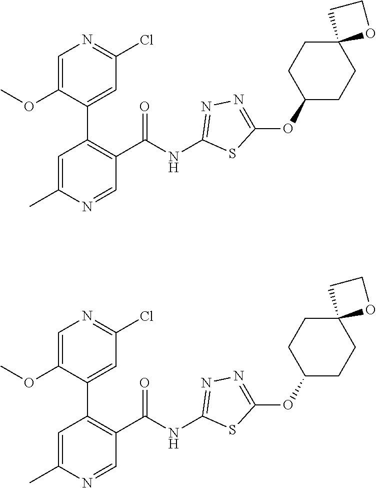 [SMILES: COc1cnc(Cl)cc1-c1cc(C)ncc1C(=O)Nc1nnc(O[C@H]2CC[C@]3(CCO3)CC2)s1 ; COc1cnc(Cl)cc1-c1cc(C)ncc1C(=O)Nc1nnc(O[C@H]2CC[C@@]3(CCO3)CC2)s1]

### Step-1: S-methyl O-(1-oxaspiro(3.5)nonan-7-yl) carbonodithioate

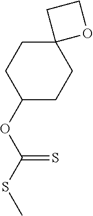

To a stirred solution of 1-oxaspiro(3.5)nonan-7-ol (100.0 mg, 0.70 mmol) in Tetrahydrofuran (THF) (3 mL) was added NaH (33.8 mg, 0.84 mmol, 60%) at 0° C. The resulting solution was stirred at 0° C. for 30 min. To the above solution was added CS₂ (80.0 mg, 1.05 mmol) at 0° C. under nitrogen. The resulting mixture was then stirred at 0° C. for 20 min. To the above solution was added MeI (150.0 mg, 1.05 mmol) at 0° C. under nitrogen. The resulting mixture was then stirred at 25° C. for 1 h. The reaction mixture was quenched by the addition of water and extracted with ethyl acetate. The combined organic layers were washed with brine, dried over anhydrous sodium sulfate, filtered, and concentrated under vacuum to afford S-methyl O-(1-oxaspiro(3.5)nonan-7-yl) carbonodithioate (160.0 mg, crude) as yellow oil.

### Step-2: O-(1-oxaspiro(3.5)nonan-7-yl) hydrazinecarbothioate

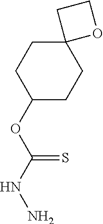 [SMILES: NNC(=S)OC1CCC2(CCO2)CC1]

A mixture of S-methyl O-(1-oxaspiro(3.5)nonan-7-yl) carbonodithioate (160.0 mg, 0.69 mmol), hydrazinium hydroxide (43.1 mg, 0.69 mmol) in Methanol (3 mL) was stirred at 25° C. for 2 h under nitrogen. The reaction mixture was quenched by the addition of water and extracted with ethyl acetate. The combined organic layers were washed with brine, dried over anhydrous sodium sulfate, filtered, and concentrated under vacuum to afford O-(1-oxaspiro(3.5)nonan-7-yl) hydrazinecarbothioate (170.0 mg, crude) as yellow oil. MS (ESI) calc'd for (C₉H₁₆N₂O₂S) (M+1)⁺, 217.1. found 217.1.

### Step-3: 5-((1-oxaspiro(3.5)nonan-7-yl)oxy)-1,3,4-thiadiazol-2-amine

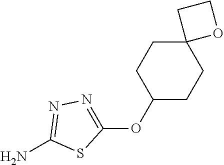 [SMILES: Nc1nnc(OC2CCC3(CCO3)CC2)s1]

To a mixture of O-(1-oxaspiro(3.5)nonan-7-yl) hydrazinecarbothioate (170.0 mg, 0.78 mmol) in Methanol (3 mL) were added TEA (159.0 mg, 1.57 mmol) and cyanic bromide (100.0 mg, 0.94 mmol) at 25° C. The resulting solution was stirred at 25° C. for 1 h under nitrogen before concentrated under vacuum. The resulting residue was dissolved in DMF (1 mL) which was applied to a 20 g C18 column and purified by Combi Flash (Biotage Isolera Prime), eluted with 5˜30% acetonitrile in water within 20 min to afford 5-((1-oxaspiro(3.5)nonan-7-yl)oxy)-1,3,4-thiadiazol-2-amine (89.0 mg, 45% over three steps) as a yellow solid. MS (ESI) calc'd for (C₁₀H₁₅N₃O₂S) (M+1)⁺, 242.2. found 242.2.

### Step-4: N-(5-((1-oxaspiro(3.5)nonan-7-yl)oxy)-1,3,4-thiadiazol-2-yl)-2′-chloro-5′-methoxy-6-methyl-(4,4′-bipyridine)-3-carboxamide

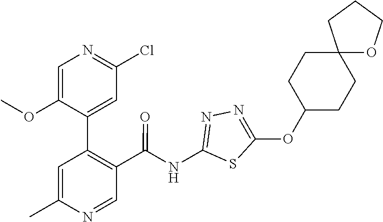 [SMILES: COc1cnc(Cl)cc1-c1cc(C)ncc1C(=O)Nc1nnc(OC2CCC3(CCCO3)CC2)s1]

To a stirred solution of Intermediate G (81.0 mg, 0.29 mmol), 5-((1-oxaspiro(3.5)nonan-7-yl)oxy)-1,3,4-thiadiazol-2-amine (70.0 mg, 0.29 mmol) and 1-methylimidazole (119.0 mg, 1.45 mmol) in Acetonitrile (1 mL). To the above was added a solution of N-(chloro(dimethylamino)methylene)-N-methylmethanaminium hexafluorophosphate(V) (90.0 mg, 0.32 mmol) in acetonitrile (1 mL) at 25° C. under nitrogen. The resulting mixture was stirred at 25° C. for 2 h. The resulting residue was dissolved in DMF (1 mL) which was applied to a 20 g C18 column and purified by Combi Flash (Biotage Isolera Prime), eluted with 5˜60% acetonitrile in water within 30 min to afford N-(5-((1-oxaspiro(3.5)nonan-7-yl)oxy)-1,3,4-thiadiazol-2-yl)-2′-chloro-5′-methoxy-6-methyl-(4,4′-bipyridine)-3-carboxamide (80.0 mg, 52%) as a yellow solid. MS (ESI) calc'd for (C₂₃H₂₄ClN₅O₄S) (M+1)⁺, 502.1. found 502.1.

### Step-5: N-(5-(((4s, 7s)-1-oxaspiro(3.5)nonan-7-yl)oxy)-1,3,4-thiadiazol-2-yl)-2′-chloro-5′-methoxy-6-methyl-(4,4′-bipyridine)-3-carboxamide and N-(5-(((4r,7r)-1-oxaspiro(3.5)nonan-7-yl)oxy)-1,3,4-thiadiazol-2-yl)-2′-chloro-5′-methoxy-6-methyl-(4,4′-bipyridine)-3-carboxamide

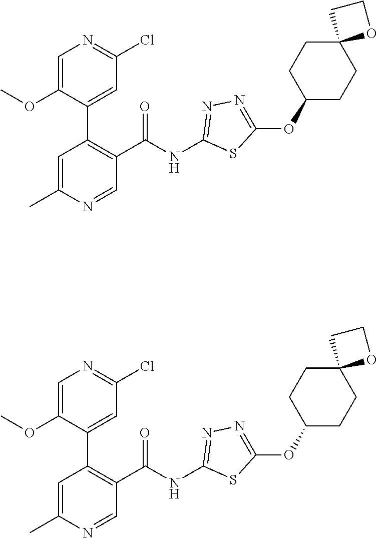 [SMILES: COc1cnc(Cl)cc1-c1cc(C)ncc1C(=O)Nc1nnc(O[C@H]2CC[C@]3(CCO3)CC2)s1 ; COc1cnc(Cl)cc1-c1cc(C)ncc1C(=O)Nc1nnc(O[C@H]2CC[C@@]3(CCO3)CC2)s1]

The racemic compound of N-(5-((1-oxaspiro(3.5)nonan-7-yl)oxy)-1,3,4-thiadiazol-2-yl)-2′-chloro-5′-methoxy-6-methyl-(4,4′-bipyridine)-3-carboxamide (80 mg) was separated by prep-chiral HPLC with the following condition: (Column: CHIRALPAK IE, 2*25 cm, 5 μm; Mobile Phase A: Hex (0.2% FA)—HPLC, Mobile Phase B: EtOH:DCM=1:1—HPLC; Flow rate: 17 mL/min; Gradient: 70% B to 70% B in 18 min; Wave Length: 220/254 nm; RT1 (min): 9.42; RT2 (min): 15.47; Sample Solvent: MeOH:DCM=1:1—HPLC; Injection Volume: 0.8 mL; Number Of Runs: 3) to afford N-(5-(((4s,7s)-1-oxaspiro(3.5)nonan-7-yl)oxy)-1,3,4-thiadiazol-2-yl)-2′-chloro-5′-methoxy-6-methyl-(4,4′-bipyridine)-3-carboxamide (40.2 mg, 49%) as a white solid with shorter retention time on chiral HPLC and N-(5-(((4r, 7r)-1-oxaspiro(3.5)nonan-7-yl)oxy)-1,3,4-thiadiazol-2-yl)-2′-chloro-5′-methoxy-6-methyl-(4,4′-bipyridine)-3-carboxamide (15.0 mg, 18%) as a white solid with longer retention time on chiral HPLC. The stereochemistry was not determined.

N-(5-(((4s, 7s)-1-oxaspiro(3.5)nonan-7-yl)oxy)-1,3,4-thiadiazol-2-yl)-2′-chloro-5′-methoxy-6-methyl-(4,4′-bipyridine)-3-carboxamide: MS (ESI) calc'd for (C₂₃H₂₄ClN₅O₄S) (M+1)⁺, 502.2. found, 502.2. ¹1H NMR (400 MHz, DMSO-d₆) δ 12.86 (s, 1H), 8.80 (s, 1H), 8.17 (s, 1H), 7.53 (s, 1H), 7.43 (s, 1H), 4.95-4.850 (m, 1H), 4.37 (t, J=7.6 Hz, 2H), 3.64 (s, 3H), 2.59 (s, 3H), 2.34 (t, J=7.6 Hz, 2H), 2.01-1.64 (m, 8H).

N-(5-(((4r,7r)-1-oxaspiro(3.5)nonan-7-yl)oxy)-1,3,4-thiadiazol-2-yl)-2′-chloro-5′-methoxy-6-methyl-(4,4′-bipyridine)-3-carboxamide: MS (ESI) calc'd for (C₂₃H₂₄ClN₅O₄S) (M+1)⁺, 502.2. found, 502.2. ¹H NMR (400 MHz, DMSO-d₆) δ 12.86 (s, 1H), 8.81 (s, 1H), 8.17 (s, 1H), 7.53 (s, 1H), 7.42 (s, 1H), 4.96-4.89 (m, 1H), 4.38 (t, J=7.6 Hz, 2H), 3.64 (s, 3H), 2.59 (s, 3H), 2.36 (t, J=7.6 Hz, 2H), 2.14-1.86 (m, 4H), 1.76-1.66 (m, 4H).

### Example 196

### 2′-chloro-6-cyclopropoxy-5′-methoxy-N-(5-methoxy-1,3,4-thiadiazol-2-yl)-(4,4′-bipyridine)-3-carboxamide

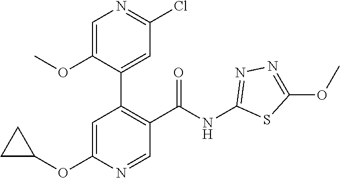 [SMILES: COc1nnc(NC(=O)c2cnc(OC3CC3)cc2-c2cc(Cl)ncc2OC)s1]

### Step-1: 2,4-dichloro-5-(ethoxycarbonyl)pyridine 1-oxide

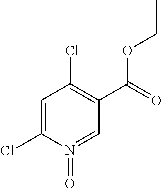

To a stirred solution of ethyl 4,6-dichloronicotinate (10.0 g, 45.66 mmol) in DCM (100 mL) were added TFAA (19.2 g, 90.89 mmol) and urea H₂O₂ (8.6 g, 90.89 mmol) at 25° C. under nitrogen. The mixture was stirred at 25° C. for 2 h. The resulting mixture was diluted with DCM (100 mL). The combined organic layers were washed with sat. Na₂CO₃ aqueous solution and brine, dried over anhydrous sodium sulfate, filtered and concentrated under vacuum. The resulting residue was purified by Combi Flash (Biotage Isolera Prime) which applied to 120 g silica gel column and eluted with 0˜48% ethyl acetate in petroleum ether within 30 min to afford 2,4-dichloro-5-(ethoxycarbonyl)pyridine 1-oxide (8.9 g, 75%) as a white solid. MS (ESI) calculated for (C₈H₇Cl₂NO₃) (M+1)⁺, 236.0. found, 236.0.

### Step-2: 4-chloro-2-cyclopropoxy-5-(ethoxycarbonyl)pyridine 1-oxide

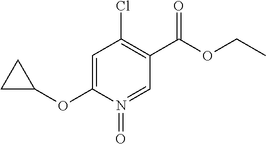 [SMILES: CCOC(=O)c1c[n+]([O-])c(OC2CC2)cc1Cl]

To a stirred solution of 2,4-dichloro-5-(ethoxycarbonyl)pyridine 1-oxide (5.0 g, 21.18 mmol) in THF (50 mL) was added NaH (1.0 g, 25.00 mmol, 60%) in portions at 0° C. The resulting solution was stirred at 0° C. for 40 min. To the above solution was added cyclopropanol (1.4 g, 24.14 mmol) at 0° C. under nitrogen. The resulting solution was stirred at 25° C. for 2 h. The reaction mixture was quenched by water and extracted with ethyl acetate. The combined organic layers were washed with brine, dried over anhydrous sodium sulfate, filtered, and concentrated under vacuum. The resulting residue was dissolved in DMF (5 mL) which was applied to a 120 g C18 column and purified by Combi Flash (Biotage Isolera Prime), eluted with 5˜55% acetonitrile in water within 30 min to afford 4-chloro-2-cyclopropoxy-5-(ethoxycarbonyl)pyridine 1-oxide (2.9 g, 53%) as a white solid. MS (ESI) calculated for (C₁₁H₁₂ClNO₄) (M+1)⁺, 258.0. found, 258.0.

### Step-3: ethyl 4-chloro-6-cyclopropoxynicotinate

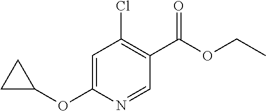 [SMILES: CCOC(=O)c1cnc(OC2CC2)cc1Cl]

To a stirred solution of 4-chloro-2-cyclopropoxy-5-(ethoxycarbonyl)pyridine 1-oxide (2.9 g, 112.84 mmol) in HOAc (30 mL) was added Fe (2.5 g, 446.42 mmol) at 25° C. The resulting solution was stirred at 60° C. for 16 hr under nitrogen. The suspension was filtered. The filter cake was washed with ethyl acetate. The filtrate was collected and dried under vacuum. The resulting residue was dissolved in DMF (5 mL) which was applied to a 80 g C18 column and purified by Combi Flash (Biotage Isolera Prime), eluted with 15˜85% acetonitrile in water within 30 min to afford ethyl 4-chloro-6-cyclopropoxynicotinate (1.5 g, 55%) as a yellow solid. MS (ESI) calculated for (C₁₁H₁₂ClNO₃) (M+1)⁺, 242.1. found, 242.0.

### Step-4: ethyl 2′-chloro-6-cyclopropoxy-5′-methoxy-(4,4′-bipyridine)-3-carboxylate

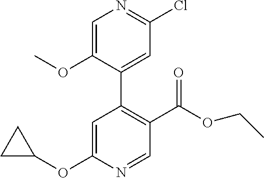 [SMILES: CCOC(=O)c1cnc(OC2CC2)cc1-c1cc(Cl)ncc1OC]

To a solution of ethyl 4-chloro-6-cyclopropoxynicotinate (1.0 g, 4.15 mmol) in dioxane (5 mL) were added Pd(dtbpf)Cl₂ (269.7 mg, 0.41 mmol), K₂CO₃ (1.7 g, 12.32 mmol) and (2-chloro-5-methoxypyridin-4-yl)boronic acid (1.2 g, 6.41 mmol) at 25° C. The resulting solution was stirred at 80° C. for 16 h. The reaction mixture was diluted with water and extracted with ethyl acetate. The combined organic layers were washed with brine, dried over anhydrous sodium sulfate, filtered, and concentrated under vacuum. The resulting residue was dissolved in DMF (3 mL) which was applied to a 40 g C18 column and purified by Combi Flash (Biotage Isolera Prime), eluted with 5˜80% acetonitrile in water within 30 min to afford ethyl 2′-chloro-6-cyclopropoxy-5′-methoxy-(4,4′-bipyridine)-3-carboxylate (500.0 mg, 34%) as a yellow solid. MS (ESI) calculated for (C₁₇H₁₇ClN₂O₄) (M+1)⁺, 349.1. found, 349.1.

### Step-5: 2′-chloro-6-cyclopropoxy-5′-methoxy-(4,4′-bipyridine)-3-carboxylic Acid

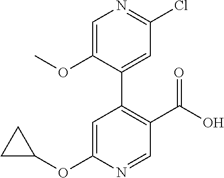 [SMILES: COc1cnc(Cl)cc1-c1cc(OC2CC2)ncc1C(=O)O]

To a stirred solution of ethyl 2′-chloro-6-cyclopropoxy-5′-methoxy-(4,4′-bipyridine)-3-carboxylate (200.0 mg, 0.57 mmol) in Tetrahydrofuran (THF) (1 mL) was added LiOH (27.5 mg, 1.14 mmol) in Water (1 mL) at 0° C. The resulting solution was stirred at room temperature for 1 h. The organic solvent was removed under vacuum. The aqueous layer was acidified with citric acid to pH ˜5 and extracted with ethyl acetate. The combined organic layers were washed with brine, dried over anhydrous sodium sulfate, filtered, and concentrated under vacuum to afford 2′-chloro-6-cyclopropoxy-5′-methoxy-(4,4′-bipyridine)-3-carboxylic acid (173 mg, crude) as a yellow solid. MS (ESI) calc'd for (C₁₅H₁₃ClN₂O₄) (M+1)⁺, 321.1. found 321.1.

### Step-6: 2′-chloro-6-cyclopropoxy-5′-methoxy-N-(5-methoxy-1,3,4-thiadiazol-2-yl)-(4,4′-bipyridine)-3-carboxamide

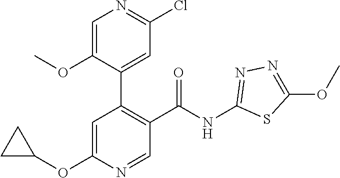 [SMILES: COc1nnc(NC(=O)c2cnc(OC3CC3)cc2-c2cc(Cl)ncc2OC)s1]

To a solution of 2′-chloro-6-cyclopropoxy-5′-methoxy-(4,4′-bipyridine)-3-carboxylic acid (120.0 mg, 0.37 mmol) in Acetonitrile (0.5 mL) were added 5-methoxy-1,3,4-thiadiazol-2-amine (49.1 mg, 0.37 mmol) and NMI (153.0 mg, 1.87 mmol) at 20° C. under nitrogen. To the above solution was added TCFH (105.0 mg, 0.37 mmol) in Acetonitrile (0.5 mL) at 25° C. under nitrogen. The resulting mixture was then stirred at 25° C. for 1 hour. The resulting residue was dissolved in DMF (2 mL) which was applied to a 20 g C18 column and purified by Combi Flash (Biotage Isolera Prime), eluted with 5˜75% acetonitrile in water within 25 min to afford 2′-chloro-6-cyclopropoxy-5′-methoxy-N-(5-methoxy-1,3,4-thiadiazol-2-yl)-(4,4′-bipyridine)-3-carboxamide (62.3 mg, 37%) as a white solid. MS (ESI) calc'd for (C₁₈H₁₆ClN₅O₄S) (M+1)⁺, 434.1. found 434.1. ¹H NMR (400 MHz, DMSO-d₆) δ 12.80 (s, 1H), 8.62 (s, 1H), 8.16 (s, 1H), 7.54 (s, 1H), 6.99 (s, 1H), 4.41-4.31 (m, 1H), 4.08 (s, 3H), 3.64 (s, 3H), 0.89-0.77 (m, 2H), 0.80-0.70 (m, 2H).

### Example 207 and 208

### N-(5-(((S)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide and N-(5-(((R)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide

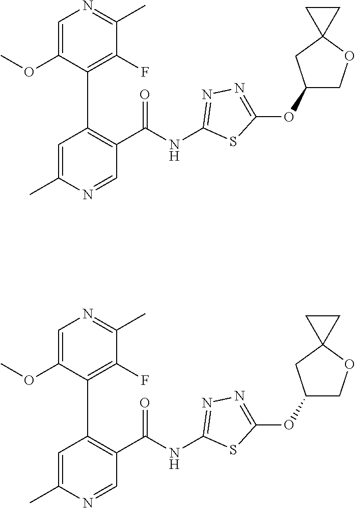 [SMILES: COc1cnc(C)c(F)c1-c1cc(C)ncc1C(=O)Nc1nnc(O[C@@H]2COC3(CC3)C2)s1 ; COc1cnc(C)c(F)c1-c1cc(C)ncc1C(=O)Nc1nnc(O[C@H]2COC3(CC3)C2)s1]

### Step-1: N-(5-((4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide

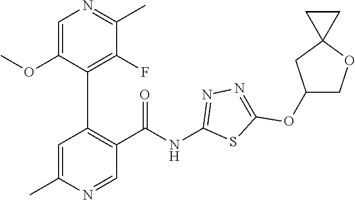 [SMILES: COc1cnc(C)c(F)c1-c1cc(C)ncc1C(=O)Nc1nnc(OC2COC3(CC3)C2)s1]

To a stirred solution of 3′-fluoro-5′-methoxy-2′,6-dimethyl-(4,4′-bipyridine)-3-carboxylic acid (259.0 mg, 0.94 mmol, Example 61, Step 2) in acetonitrile (2 mL) were sequentially added 5-((4-oxaspiro(2.4)heptan-6-yl)oxy)-1,3,4-thiadiazol-2-amine (200.0 mg, 0.94 mmol, Example 95 and 107, Step 3) and 1-methyl-1H-imidazole (385.0 mg, 4.69 mmol) at 25° C. Then N,N,N′,N′-Tetramethylchloroformamidinium hexafluorophosphate (263.0 mg, 0.94 mmol) in acetonitrile (1 mL) was added to the above mixture at 25° C. The resulting solution was stirred at 25° C. for 2 h. The resulting residue was dissolved in acetonitrile (3 mL) which was applied to a 20 g C18 column and purified by Combi Flash (Biotage Isolera Prime), eluted with 5˜42% acetonitrile in water within 30 min to afford N-(5-((4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide (290.0 mg, 63%) as a white solid. MS (ESI) calculated for (C₂₂H₂₂FN₅O₄S) (M+1)⁺, 472.1. found, 472.2.

### Step-2: N-(5-(((S)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide and N-(5-(((R)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide

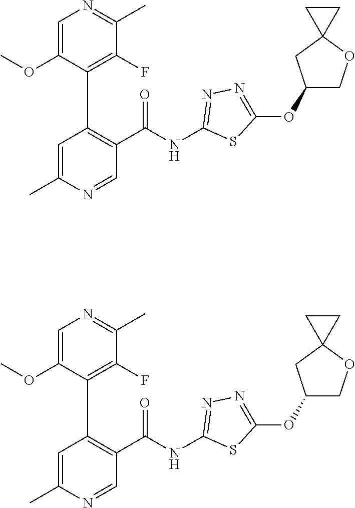 [SMILES: COc1cnc(C)c(F)c1-c1cc(C)ncc1C(=O)Nc1nnc(O[C@@H]2COC3(CC3)C2)s1 ; COc1cnc(C)c(F)c1-c1cc(C)ncc1C(=O)Nc1nnc(O[C@H]2COC3(CC3)C2)s1]

N-(5-((4-Oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide (200.0 mg) was separated by prep-chiral HPLC with the following condition: (Column: CHIRAL ART Cellulose-SB, 2*25 cm, 5 μm; Mobile Phase A: Hex (0.2% FA)—HPLC, Mobile Phase B: EtOH:DCM=1:1—HPLC; Flow rate: 20 mL/min; Gradient: 20% B to 20% B in 10 min; Wave Length: 220/254 nm; RT1 (min): 7.86; RT2 (min): 9.68; Sample Solvent: MeOH:DCM=2:1 (0.1% FA); Injection Volume: 0.8 mL; Number Of Runs: 14) to afford N-(5-(((S)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide (87.3 mg, 43.3%) as a white solid with the first peak on chiral HPLC and N-(5-(((R)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide (81.6 mg, 40%) as a white solid with the second peak on chiral HPLC. The absolute stereochemistry was not determined.

N-(5-(((S)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide: MS (ESI) calculated for (C₂₂H₂₂FN₅O₄S) (M+1)⁺, 472.1. found, 472.2. ¹H NMR (400 MHz, DMSO-d₆) δ 13.00 (s, 1H), 8.91 (s, 1H), 8.18 (s, 1H), 7.40 (s, 1H), 5.59-5.56 (m, 1H), 4.02-4.00 (m, 2H), 3.69 (s, 3H), 2.59 (s, 3H), 2.49-2.44 (m, 1H), 2.43 (s, 3H), 2.15-2.14 (m, 1H), 0.82-0.51 (m, 4H).

N-(5-(((R)-4-oxaspiro(2.4)heptan-6-yl)oxy)-1,3,4-thiadiazol-2-yl)-3′-fluoro-5′-methoxy-2′,6-dimethyl-(4,4′-bipyridine)-3-carboxamide: MS (ESI) calculated for (C₂₂H₂₂FN₅O₄S) (M+1)⁺, 472.1. found, 472.2. ¹H NMR (400 MHz, DMSO-d₆) δ13.00 (s, 1H), 8.91 (s, 1H), 8.18 (s, 1H), 7.40 (s, 1H), 5.59-5.56 (m, 1H), 4.02-4.00 (m, 2H), 3.69 (s, 3H), 2.59 (s, 3H), 2.49-2.44 (m, 1H), 2.43 (s, 3H), 2.15-2.14 (m, 1H), 0.82-0.53 (m, 4H).

### Example 215

### 2′-chloro-6-(cyclopropoxymethyl)-5′-methoxy-N-(5-methoxy-1,3,4-thiadiazol-2-yl)-(4,4′-bipyridine)-3-carboxamide

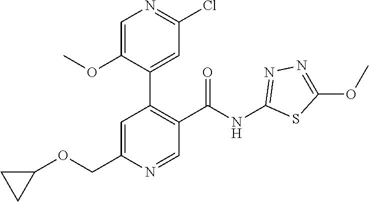 [SMILES: COc1nnc(NC(=O)c2cnc(COC3CC3)cc2-c2cc(Cl)ncc2OC)s1]

### Step-1: ethyl 4-chloro-6-(chloromethyl)nicotinate

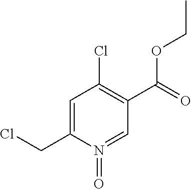

To a stirred solution of ethyl 4-chloro-6-(hydroxymethyl)nicotinate (2.0 g, 9.28 mmol) in Dichloromethane (30 mL) was added SOCl₂ (1.6 g, 13.91 mmol) at 0° C. The resulting solution was then stirred at 25° C. for 2 h. The reaction mixture was quenched by the addition of saturated sodium bicarbonate solution and extracted with Dichloromethane. The combined organic layers were washed with brine, dried over anhydrous sodium sulfate, filtered, and concentrated under vacuum to afford ethyl 4-chloro-6-(chloromethyl)nicotinate (1.8 g, crude) as yellow oil, which was used in the next step without further purification. MS (ESI) calc'd for (C₉H₉Cl₂NO₂) (M+1)⁺, 234.0. found 234.1.

### Step-2: ethyl 4-chloro-6-(cyclopropoxymethyl)nicotinate

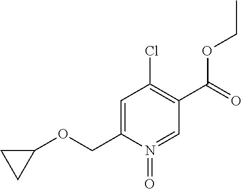 [SMILES: CCOC(=O)c1c[n+]([O-])c(COC2CC2)cc1Cl]

To a stirred solution of cyclopropanol (223.0 mg, 3.84 mmol) in Tetrahydrofuran (20 mL) was added NaH (185.0 mg, 4.61 mmol, 60%) in portions at 0° C. The reaction mixture was stirred at 0° C. for 0.5 h. Then ethyl 4-chloro-6-(chloromethyl)nicotinate (900.0 mg, 3.84 mmol) in Tetrahydrofuran (20 mL) was added to the above mixture at 0° C. The resulting solution was then stirred at 25° C. for 1.5 h. The reaction mixture was quenched by the addition of water and extracted with ethyl acetate. The combined organic layers were washed with brine, dried over anhydrous sodium sulfate, filtered, and concentrated under vacuum. The resulting residue was dissolved in DMF (6 mL) which was applied to a 80 g C18 column and purified by Combi Flash (Biotage Isolera Prime), eluted with 5˜50% acetonitrile in water within 25 min to afford ethyl 4-chloro-6-(cyclopropoxymethyl)nicotinate (330.0 mg, 30%) as a yellow solid. MS (ESI) calc'd for (C₁₂H₁₄ClNO₃) (M+1)⁺, 256.0. found 256.1.

### Step-3: ethyl 2′-chloro-6-(cyclopropoxymethyl)-5′-methoxy-(4,4′-bipyridine)-3-carboxylate

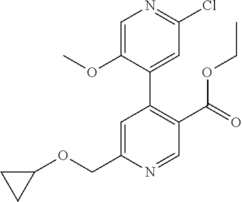 [SMILES: CCOC(=O)c1cnc(COC2CC2)cc1-c1cc(Cl)ncc1OC]

To a stirred solution of ethyl 4-chloro-6-(cyclopropoxymethyl)nicotinate (220.0 mg, 0.86 mmol) in 1,4-Dioxane (2 mL) and Water (0.4 mL) were sequentially added (2-chloro-5-methoxypyridin-4-yl)boronic acid (484.0 mg, 2.58 mmol), K₂CO₃ (357.0 mg, 2.58 mmol) and 1,1′-Bis (di-t-butylphosphino)ferrocene palladium dichloride (56.1 mg, 0.08 mmol) at 25° C. The resulting solution was stirred at 80° C. for 16 h under nitrogen atmosphere. The suspension was filtered. The filtrate was concentrated under vacuum. The residue was applied to a 40 g C18 column and purified by Combi Flash (Biotage Isolera Prime), eluted with 5˜43% acetonitrile in water within 30 min to afford ethyl 2′-chloro-6-(cyclopropoxymethyl)-5′-methoxy-(4,4′-bipyridine)-3-carboxylate (110.0 mg, 30%) as a yellow solid. MS (ESI) calc'd for (C₁₈H₁₉ClN₂O₄) (M+1)⁺, 363.1. found 363.0.

### Step-4: 2′-chloro-6-(cyclopropoxymethyl)-5′-methoxy-(4,4′-bipyridine)-3-carboxylic Acid

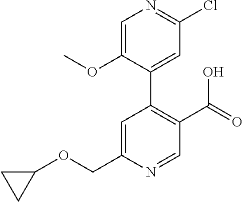 [SMILES: COc1cnc(Cl)cc1-c1cc(COC2CC2)ncc1C(=O)O]

To a stirred solution of ethyl 2′-chloro-6-(cyclopropoxymethyl)-5′-methoxy-(4,4′-bipyridine)-3-carboxylate (189.0 mg, 0.52 mmol) in Tetrahydrofuran (1.5 mL) and Water (0.5 mL) was added LiOH (37.4 mg, 1.56 mmol) at 25° C. The resulting solution was stirred at 25° C. for 2 h. The organic solvent was acidified with citric acid to pH 5˜6. The organic solvent was applied to a 20 g C18 column and purified by Combi Flash (Biotage Isolera Prime), eluted with 5˜40% acetonitrile in water within 30 min to afford 2′-chloro-6-(cyclopropoxymethyl)-5′-methoxy-(4,4′-bipyridine)-3-carboxylic acid (80.0 mg, 8%) as a white solid. MS (ESI) calc'd for (C₁₆H₁₅ClN₂O₄) (M+1)⁺, 335.0. found 335.0.

### Step-5: 2′-chloro-6-(cyclopropoxymethyl)-5′-methoxy-N-(5-methoxy-1,3,4-thiadiazol-2-yl)-(4,4′-bipyridine)-3-carboxamide

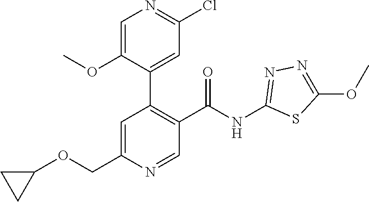 [SMILES: COc1nnc(NC(=O)c2cnc(COC3CC3)cc2-c2cc(Cl)ncc2OC)s1]

To a stirred solution of 2′-chloro-6-(cyclopropoxymethyl)-5′-methoxy-(4,4′-bipyridine)-3-carboxylic acid (30.0 mg, 0.09 mmol) in Acetonitrile (1 mL) were sequentially added 1-methyl-1H-imidazole (36.8 mg, 0.44 mmol), 5-methoxy-1,3,4-thiadiazol-2-amine (14.1 mg, 0.10 mmol) and TCFH (25.1 mg, 0.09 mmol) at 25° C. The resulting solution was stirred at 25° C. for 2 h. The organic solvent was acidified with citric acid to pH ˜6. The organic solvent (2 mL) was applied to a 20 g C18 column and purified by Combi Flash (Biotage Isolera Prime), eluted with 5˜60% acetonitrile in water within 30 min to afford 2′-chloro-6-(cyclopropoxymethyl)-5′-methoxy-N-(5-methoxy-1,3,4-thiadiazol-2-yl)-(4,4′-bipyridine)-3-carboxamide (20.5 mg, 50%) as a white solid. MS (ESI) calc'd for (C₁₉H₁₈ClN₅O₄S) (M+1)⁺, 448.0. found 448.0. ¹H NMR (400 MHz, DMSO-d₆) δ 12.20 (s, 1H), 8.52 (s, 1H), 8.15 (s, 1H), 7.41 (s, 1H), 7.31 (s, 1H), 4.20-4.00 (m, 6H), 3.90 (s, 3H), 0.92-0.78 (m, 4H).

### Example 216 and 217

### N-(5-(((1S,2R,4R)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide and N-(5-(((1R,2S,4S)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide

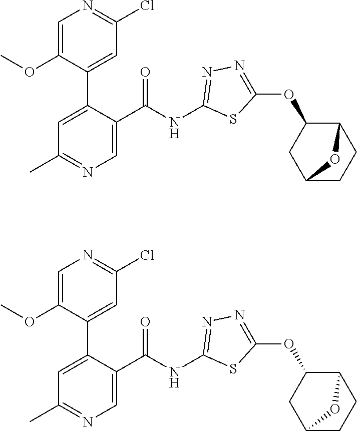 [SMILES: COc1cnc(Cl)cc1-c1cc(C)ncc1C(=O)Nc1nnc(O[C@@H]2C[C@H]3CC[C@@H]2O3)s1 ; COc1cnc(Cl)cc1-c1cc(C)ncc1C(=O)Nc1nnc(O[C@H]2C[C@@H]3CC[C@H]2O3)s1]

### Step-1: rac-O-((1R,2S,4S)-7-oxabicyclo(2.2.1) heptan-2-yl)S-methyl Carbonodithioate

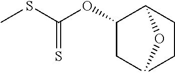

To a stirred solution of rac-(1R,2S,4S)-7-oxabicyclo(2.2.1)heptan-2-ol (750 mg, 6.57 mmol) in Tetrahydrofuran (15 mL) under nitrogen at 0° C. was added sodium hydride (315 mg, 13.14 mmol) portionwise during 5 min. After addition reaction mixture was stirred at room temperature for 30 min. After 30 min to the above reaction mixture were added carbon disulfide (0.792 mL, 13.14 mmol) followed by methyl iodide (0.411 mL, 6.57 mmol)) at room temperature. Then the reaction mixture was stirred at room temperature for additional 30 min. The reaction was quenched with cold water extracted with ethyl acetate (2×40 mL). The combined organic layers were dried over sodium sulphate, filtered and concentrated under vacuum to afford rac-O-((1R,2S,4S)-7-oxabicyclo(2.2.1) heptan-2-yl)S-methyl carbonodithioate (1.3 g, 6.29 mmol, 96% yield) as yellow liquid. MS (ESI) calculated for (C₈H₁₂O₂S₂) (M+1)⁺, 205.04. found, 205.0.

### Step-2: rac-O-((1R,2S,4S)-7-oxabicyclo(2.2.1)heptan-2-yl) hydrazinecarbothioate

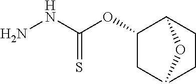

To a stirred solution of rac-O-((1R,2S,4S)-7-oxabicyclo(2.2.1)heptan-2-yl)S-methyl carbonodithioate (1.3 g, 6.24 mmol) in Methanol (5 mL) was added hydrazine monohydrate (0.343 g, 6.86 mmol) at room temperature. The reaction mixture was stirred at room temperature for 1 hr. The organic solvent was removed under vacuum. The resulting residue was diluted with water (40 mL) and extracted with ethyl acetate (2×30 mL). The organic layer was dried over anhydrous sodium sulphate, filtered concentrated under reduced pressure to afford rac-O-((1R,2S,4S)-7-oxabicyclo(2.2.1)heptan-2-yl) hydrazinecarbothioate (1 g, 5.29 mmol, 85% yield) as pale yellow solid.

MS (ESI) calculated for (C₇H₁₂N₂O₂S) (M+1)⁺, 189.07. found, 189.2.

### Step-3: rac-5-(((1R,2S,4S)-7-oxabicyclo(2.2.1)heptan-2-yl)oxy)-1,3,4-thiadiazol-2-amine

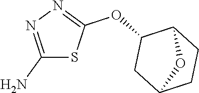 [SMILES: Nc1nnc(O[C@H]2C[C@@H]3CC[C@H]2O3)s1]

To a stirred solution of rac-O-((1R,2S,4S)-7-oxabicyclo(2.2.1)heptan-2-yl) hydrazinecarbothioate (1 g, 5.31 mmol) in Ethanol (5 mL) were added triethylamine (0.740 mL, 5.31 mmol) followed by cyanogen bromide (0.563 g, 5.31 mmol) at room temperature. The reaction mixture was stirred at room temperature for 30 mins. The organic solvent was removed under vacuum. The resulting residue was diluted with water (30 mL) and extracted with 10% MeOH in ethyl acetate (2×50 mL). The organic layer dried over anhydrous sodium sulphate, filtered concentrated under vacuum to get the crude product as a brown gum.

The resulting residue was dissolved in ACN (4 mL) and THF (2 mL) and liquid injected to a 100 g C18 column and purified by GRACE revelleris X2, eluted with 0˜100% acetonitrile in water within 40 min to afford rac-5-(((1R,2S,4S)-7-oxabicyclo(2.2.1)heptan-2-yl)oxy)-1,3,4-thiadiazol-2-amine (500 mg, 2.258 mmol, 42.5% yield) as a brown solid.

MS (ESI) calculated for (C₈H₁₁N₃O₂S) (M+1)⁺, 214.07. found, 214.2.

### Step-4: rac-N-(5-(((1R,2S,4S)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide

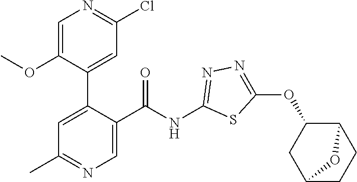 [SMILES: COc1cnc(Cl)cc1-c1cc(C)ncc1C(=O)Nc1nnc(O[C@H]2C[C@@H]3CC[C@H]2O3)s1]

To a stirred solution of Intermediate G (200 mg, 0.718 mmol) in Acetonitrile (3 mL) and N,N-Dimethylformamide (0.6 mL) were added 1-methyl-1H-imidazole (177 mg, 2.153 mmol), Chloro-N,N,N′,N′-tetramethylformamidinium hexafluorophosphate (221 mg, 0.789 mmol) and rac-5-(((1R,2S,4S)-7-oxabicyclo(2.2.1)heptan-2-yl)oxy)-1,3,4-thiadiazol-2-amine (153 mg, 0.718 mmol) at room temperature. The reaction mixture was stirred at room temperature for 3 h. The reaction mixture was quenched with cold water and extracted with ethyl acetate (2×10 mL). The combined organic layers were washed with brine, dried over anhydrous sodium sulfate, filtered, and concentrated under vacuum to get the crude product as a brown gum.

The resulting residue was dissolved in ACN (4 mL) and THF (2 mL) and liquid injected to a 100 g C18 column and purified by GRACE revelleris X2, eluted with 0˜100% acetonitrile in water within 40 min to afford rac-N-(5-(((1R,2S,4S)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide (200 mg, 58%) as a white solid.

MS (ESI) calculated for (C₂₁H₂₀ClN₅O₄S) (M+1)⁺, 474.1. found, 474.2.

### Step-5: N-(5-(((1S,2R,4R)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide and N-(5-(((1R,2S,4S)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide

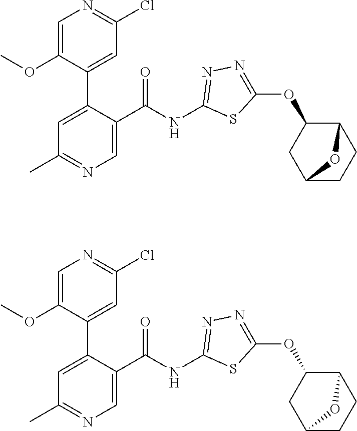 [SMILES: COc1cnc(Cl)cc1-c1cc(C)ncc1C(=O)Nc1nnc(O[C@@H]2C[C@H]3CC[C@@H]2O3)s1 ; COc1cnc(Cl)cc1-c1cc(C)ncc1C(=O)Nc1nnc(O[C@H]2C[C@@H]3CC[C@H]2O3)s1]

The rac-N-(5-(((1R,2S,4S)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide (200 mg) was separated by prep-chiral SFC with the following condition: (Column: YMC Cellulose SZ (250*30) mm, 5 μm; Mobile Phase A: CO₂, Mobile Phase B: 0.5% Isopropyl Amine in Methanol; Gradient: isocratic 40% B; Column Temperature (° C.): 35; Back Pressure (bar): 100; Wave Length: 220 nm; RT1 (min): 3.42; RT2 (min): 4.03; Sample Solvent: DCM/MeOH-HPLC; Injection Volume: 0.9 mL/Injection; Cycle time: 7.5 min) to afford N-(5-(((1S,2R,4R)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide (50 mg, 0.105 mmol, 14.67% yield) as a pale brown solid with the first peak on chiral SFC with shorter retention time and N-(5-(((1R,2S,4S)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide (52 mg, 0.110 mmol, 15.26% yield) as a pale brown solid with the second peak on chiral SFC with longer retention time. The absolute stereochemistry was not determined.

N-(5-(((1S,2R,4R)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide: MS (ESI) calculated for (C₂₁H₂₀ClN₅O₄S) (M+1)⁺, 474.1. found, 474.0. 1H-NMR (400 MHz, DMSO-d⁶): δ 12.92 (s, 1H), 8.81 (s, 1H), 8.17 (s, 1H), 7.54 (s, 1H), 7.43 (s, 1H), 5.00 (dd, J=2.4 Hz, 7.2 Hz, 1H), 4.66 (d, J=5.6 Hz, 1H), 4.62 (t, J=5.2 Hz, 1H), 3.63 (s, 3H), 2.59 (s, 3H), 2.12-2.05 (m, 1H), 1.72-1.69 (m, 1H), 1.61-1.58 (m, 1H), 1.53-1.38 (m, 3H).

N-(5-(((1R,2S,4S)-7-oxabicyclo(2.2.1)heptan-2-yl)oxy)-1,3,4-thiadiazol-2-yl)-2′-chloro-5′-methoxy-6-methyl-(4,4′-bipyridine)-3-carboxamide: MS (ESI) calculated for (C₂₁H₂₀ClN₅O₄S) (M+1)⁺, 474.1. found, 474.0. 1H-NMR (400 MHz, DMSO-d⁶): δ 12.92 (s, 1H), 8.81 (s, 1H), 8.17 (s, 1H), 7.54 (s, 1H), 7.42 (s, 1H), 5.00 (dd, J=2.0 Hz, 6.8 Hz, 1H), 4.66 (d, J=6.0 Hz, 1H), 4.62 (t, J=4.8 Hz, 1H), 3.63 (s, 3H), 2.59 (s, 3H), 2.12-2.05 (m, 1H), 1.72-1.69 (m, 1H), 1.61-1.58 (m, 1H), 1.53-1.38 (m, 3H).

### Example 218 and 219

### N-(5-(((1S,2R,4R)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide and N-(5-(((1R,2S,4S)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide

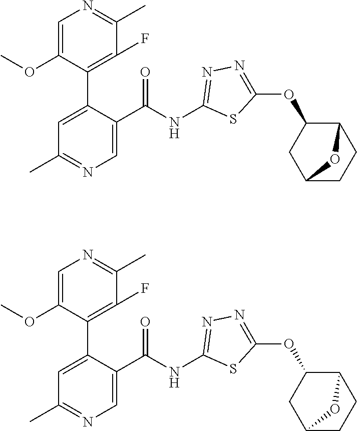 [SMILES: COc1cnc(C)c(F)c1-c1cc(C)ncc1C(=O)Nc1nnc(O[C@@H]2C[C@H]3CC[C@@H]2O3)s1 ; COc1cnc(C)c(F)c1-c1cc(C)ncc1C(=O)Nc1nnc(O[C@H]2C[C@@H]3CC[C@H]2O3)s1]

### Step 1: rac-N-(5-(((1R,2S,4S)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide

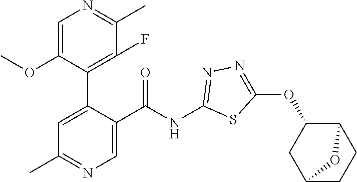 [SMILES: COc1cnc(C)c(F)c1-c1cc(C)ncc1C(=O)Nc1nnc(O[C@H]2C[C@@H]3CC[C@H]2O3)s1]

To a stirred solution of 3′-fluoro-5′-methoxy-2′,6-dimethyl-(4,4′-bipyridine)-3-carboxylic acid (80 mg, 0.290 mmol, Example 61, Step 2) in Acetonitrile (3 mL) and N,N-Dimethylformamide (0.6 mL) were added 1-methyl-1H-imidazole (71.3 mg, 0.869 mmol), Chloro-N,N,N′,N′-tetramethylformamidinium hexafluorophosphate (89 mg, 0.319 mmol) and rac-5-(((1R,2S,4S)-7-oxabicyclo(2.2.1)heptan-2-yl)oxy)-1,3,4-thiadiazol-2-amine (61.8 mg, 0.290 mmol, Example 216 and 217, Step 3) at room temperature. The reaction mixture was stirred at room temperature for 3 h. The reaction mixture was quenched with cold water and extracted with ethyl acetate (2×10 mL). The combined organic layers were washed with brine, dried over anhydrous sodium sulfate, filtered, and concentrated under vacuum to get the crude product as a brown gum.

The resulting residue was dissolved in ACN (4 mL) and liquid injected to a 100 g C18 column and purified by GRACE revelleris X2, eluted with 0˜100% acetonitrile in (0.1 M formic acid) water within 40 min to afford rac-N-(5-(((1R,2S,4S)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide (60 mg, 44%) as a white solid. MS (ESI) calculated for (C₂₂H₂₂FN₅O₄S) (M+1)⁺, 472.15. found, 472.2.

### Step-2: N-(5-(((1S,2R,4R)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide and N-(5-(((1R,2S,4S)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide

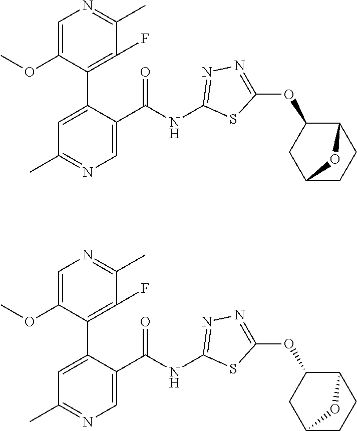 [SMILES: COc1cnc(C)c(F)c1-c1cc(C)ncc1C(=O)Nc1nnc(O[C@@H]2C[C@H]3CC[C@@H]2O3)s1 ; COc1cnc(C)c(F)c1-c1cc(C)ncc1C(=O)Nc1nnc(O[C@H]2C[C@@H]3CC[C@H]2O3)s1]

The rac-N-(5-(((1R,2S,4S)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide (combined 2 batches 90 mg) was separated by prep-chiral SFC with the following condition: (Column: Lux Amylose-1 (250*30) mm, 5 um; Mobile Phase A: CO₂, Mobile Phase B: 0.5% Isopropyl Amine in Methanol; Gradient: isocratic 40% B; Column Temperature (° C.): 35; Back Pressure (bar): 100; Total flow: 100 g/mL; Wave Length: 210 nm; RT1 (min): 2.29; RT2 (min): 3.65; Sample Solvent: MeOH-HPLC; Injection Volume: 0.6 mL/Injection; Cycle time: 6 min) to afford N-(5-(((1S,2R,4R)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide (22 mg, 0.046 mmol, 15.95% yield) as a white solid with the first peak on chiral SFC with shorter retention time and N-(5-(((1R,2S,4S)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide (25 mg, 0.052 mmol, 17.85% yield) as a light brown solid with the second peak on chiral SFC with longer retention time. The absolute stereochemistry was not determined.

N-(5-(((1S,2R,4R)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide: MS (ESI) calculated for (C₂₂H₂₂FN₅O₄S) (M+1)⁺, 472.15. found, 472.2. 1H-NMR (400 MHz, DMSO-d⁶): δ 13.0 (s, 1H), 8.90 (s, 1H), 8.18 (s, 1H), 7.40 (s, 1H), 4.99 (dd, J=2.00 Hz, 7.2 Hz, 1H), 4.65 (d, J=5.6 Hz, 1H), 4.62 (t, J=5.2 Hz, 1H), 3.68 (d, J=2.0 Hz, 3H), 2.59 (s, 3H), 2.42 (d, J=2.8 Hz, 3H), 2.11-2.03 (m, 1H), 1.73-1.56 (m, 2H), 1.55-1.36 (m, 3H).

N-(5-(((1R,2S,4S)-7-oxabicyclo(2.2.1)heptan-2-yl)oxy)-1,3,4-thiadiazol-2-yl)-3′-fluoro-5′-methoxy-2′,6-dimethyl-(4,4′-bipyridine)-3-carboxamide: MS (ESI) calculated for (C₂₂H₂₂FN₅O₄S) (M+1)⁺, 472.15. found, 472.0. 1H-NMR (400 MHz, DMSO-d⁶): δ 13.0 (s, 1H), 8.92 (s, 1H), 8.17 (s, 1H), 7.38 (s, 1H), 4.98 (dd, J=2.00 Hz, 6.80 Hz, 1H), 4.65 (d, J=6.0 Hz, 1H), 4.61 (t, J=4.8 Hz, 1H), 3.68 (s, 3H), 2.58 (s, 3H), 2.42 (d, J=3.2 Hz, 3H), 2.11-2.03 (m, 1H), 1.73-1.56 (m, 2H), 1.55-1.35 (m, 3H).

### Example 226

### (S)—N-(5-((4-oxaspiro(2.4)heptan-6-yl)oxy)-1,3,4-thiadiazol-2-yl)-2′-bromo-5′-methoxy-6-methyl-(4,4′-bipyridine)-3-carboxamide

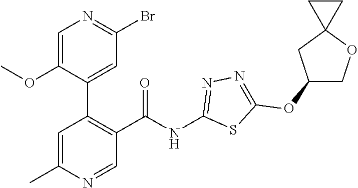 [SMILES: COc1cnc(Br)cc1-c1cc(C)ncc1C(=O)Nc1nnc(O[C@@H]2COC3(CC3)C2)s1]

### Step-1: (S)-5-((4-oxaspiro(2.4)heptan-6-yl)oxy)-1,3,4-thiadiazol-2-amine and (R)-5-((4-oxaspiro(2.4)heptan-6-yl)oxy)-1,3,4-thiadiazol-2-amine

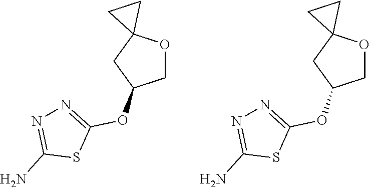 [SMILES: Nc1nnc(O[C@@H]2COC3(CC3)C2)s1 ; Nc1nnc(O[C@H]2COC3(CC3)C2)s1]

The racemic 5-((4-oxaspiro(2.4)heptan-6-yl)oxy)-1,3,4-thiadiazol-2-amine (1.8 g, 8.44 mmol, Example 95 and 107, Step 3) was separated by prep-chiral SFC with the following condition: (Column: Lux A1 (250*30) mm, 5 μm; Mobile Phase A: CO₂, Mobile Phase B: Methanol; Gradient: isocratic 30% B; Column Temperature (° C.): 35; Back Pressure (bar): 100; Wave Length: 254 nm; RT1 (min): 2.59; RT2 (min): 3.24; Sample Solvent: MeOH-HPLC (100 mL); Injection Volume: 1 mL/Injection; Cycle time: 5 min) to afford (S)-5-((4-oxaspiro(2.4)heptan-6-yl)oxy)-1,3,4-thiadiazol-2-amine (0.750 g, 3.51 mmol, 41.6% yield) as pale brown solid with the first peak on chiral SFC with shorter retention time and (R)-5-((4-oxaspiro(2.4)heptan-6-yl)oxy)-1,3,4-thiadiazol-2-amine (0.650 g, 3.02 mmol, 35.8% yield) as pale brown solid with the second peak on chiral SFC with longer retention time. The absolute stereochemistry was not determined.

(S)-5-((4-oxaspiro(2.4)heptan-6-yl)oxy)-1,3,4-thiadiazol-2-amine: MS (ESI) calculated for (C₈H₁₁N₃O₂S) (M+1)⁺, 214.07. found, 214.0. 1H-NMR (400 MHz, DMSO-d⁶): δ 6.79 (s, 2H), 5.49-5.40 (m, 1H), 4.01-3.89 (m, 2H), 2.41 (q, J=6.80 Hz, 1H), 2.08 (d, J=14.0 Hz, 1H), 0.85-0.76 (m, 1H), 0.75-0.68 (m, 1H), 0.62-0.55 (m, 1H), 0.54-0.45 (m, 1H),

(R)-5-((4-oxaspiro(2.4)heptan-6-yl)oxy)-1,3,4-thiadiazol-2-amine: MS (ESI) calculated for (C₈H₁₁N₃O₂S) (M+1)⁺, 214.07. found, 214.0. 1H-NMR (400 MHz, DMSO-d⁶): δ 6.79 (s, 2H), 5.49-5.40 (m, 1H), 4.01-3.89 (m, 2H), 2.41 (q, J=6.80 Hz, 1H), 2.08 (d, J=14.00 Hz, 1H), 0.85-0.76 (m, 1H), 0.75-0.68 (m, 1H), 0.62-0.55 (m, 1H), 0.54-0.45 (m, 1H),

### Step-2: (S)—N-(5-((4-oxaspiro(2.4)heptan-6-yl)oxy)-1,3,4-thiadiazol-2-yl)-2′-bromo-5′-methoxy-6-methyl-(4,4′-bipyridine)-3-carboxamide

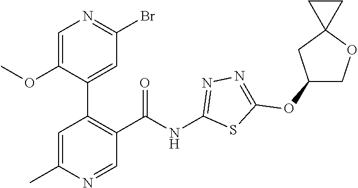 [SMILES: COc1cnc(Br)cc1-c1cc(C)ncc1C(=O)Nc1nnc(O[C@@H]2COC3(CC3)C2)s1]

To a stirred solution of 2′-bromo-5′-methoxy-6-methyl-(4,4′-bipyridine)-3-carboxylic acid (150 mg, 0.464 mmol, Example 63, Step 3), in Acetonitrile (3 mL) and N,N-Dimethylformamide (0.5 mL) were added Chloro-N,N,N′,N′-tetramethylformamidinium hexafluorophosphate (143 mg, 0.511 mmol), 1-methylimidazole (0.148 mL, 1.857 mmol), and (S)-5-((4-oxaspiro(2.4)heptan-6-yl)oxy)-1,3,4-thiadiazol-2-amine (99 mg, 0.464 mmol) at room temperature. The reaction mixture was stirred at same temperature for 3 h. The reaction mixture was diluted with water, extracted with ethyl acetate (2×20 mL). The combined organic layer dried over anhydrous sodium sulphate, filtered concentrated under vacuum to afford crude product as yellow gum.

The crude product was purified by prep-HPLC with the following condition: (Column: X-SELECT C18 (19*250) MM 5 MICRON; Mobile Phase A: 10 mM ABC in Milli Q Water 80%, Mobile Phase B: Acetonitrile 20%; Flow rate: 12 mL/min; Run time: 21 min; (Sample Solvent: THF:ACN; Injection Volume: 200 μL; number of injections: 38) to afford (S)—N-(5-((4-oxaspiro(2.4)heptan-6-yl)oxy)-1,3,4-thiadiazol-2-yl)-2′-bromo-5′-methoxy-6-methyl-(4,4′-bipyridine)-3-carboxamide (110 mg, 0.209 mmol, 45.1% yield) as an off white solid. The absolute stereochemistry was not determined.

MS (ESI) calculated for (C₂₁H₂₀BrN₅O₄S) (M+1)⁺, 518.05. found, 518.0. 1H-NMR (400 MHz, DMSO-d⁶): δ 12.93 (s, 1H), 8.80 (s, 1H), 8.18 (s, 1H), 7.64 (s, 1H), 7.43 (s, 1H), 5.63-5.58 (m, 1H), 4.08-3.95 (m, 2H), 3.63 (s, 3H), 2.59 (s, 3H), 2.50-2.45 (m, 1H), 2.15 (d, J=13.60 Hz, 1H), 0.88-0.78 (m, 1H), 0.77-0.69 (m, 1H), 0.68-0.57 (m, 1H), 0.56-0.46 (m, 1H).

### Example 227

### (R)—N-(5-((4-oxaspiro(2.4)heptan-6-yl)oxy)-1,3,4-thiadiazol-2-yl)-2′-bromo-5′-methoxy-6-methyl-(4,4′-bipyridine)-3-carboxamide

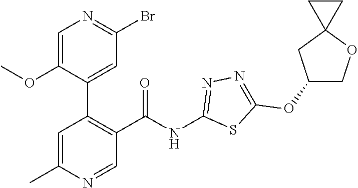 [SMILES: COc1cnc(Br)cc1-c1cc(C)ncc1C(=O)Nc1nnc(O[C@H]2COC3(CC3)C2)s1]

To a stirred solution of 2′-bromo-5′-methoxy-6-methyl-(4,4′-bipyridine)-3-carboxylic acid (150 mg, 0.464 mmol, Example 63, Step 3), in Acetonitrile (3 mL) and N,N-Dimethylformamide (0.5 mL) were added Chloro-N,N,N′,N′-tetramethylformamidinium hexafluorophosphate (195 mg, 0.696 mmol), 1-methylimidazole (0.148 mL, 1.857 mmol), and (R)-5-((4-oxaspiro(2.4)heptan-6-yl)oxy)-1,3,4-thiadiazol-2-amine (99 mg, 0.464 mmol, Example 226, Step 1) at room temperature. The reaction mixture was stirred at room temperature for 3 h. After completion, the reaction mixture was diluted with water and extracted with ethyl acetate (2×20 mL). The combined organic layer dried over anhydrous sodium sulphate, filtered concentrated under vacuum to afford the crude product as yellow gum.

The crude product was purified by prep-HPLC with the following condition: (Column: X-SELECT C18 (19*250 MM) 5 MICRON; Mobile Phase A: 10 mM ABC in Milli Q Water 80%, Mobile Phase B: Acetonitrile 20%; Flow rate: 10 mL/min; Run time: 21 min; (Sample Solvent: THF:ACN; Injection Volume: 200 μL; number of injections: 24) to afford (R)—N-(5-((4-oxaspiro(2.4)heptan-6-yl)oxy)-1,3,4-thiadiazol-2-yl)-2′-bromo-5′-methoxy-6-methyl-(4,4′-bipyridine)-3-carboxamide (75 mg, 0.144 mmol, 31.1% yield) as an off white solid. The absolute stereochemistry was not determined.

MS (ESI) calculated for (C₂₁H₂₀BrN₅O₄S) (M+1)⁺, 518.05. found, 518.0. 1H-NMR (400 MHz, DMSO-d⁶): δ 12.93 (s, 1H), 8.81 (s, 1H), 8.17 (s, 1H), 7.63 (s, 1H), 7.42 (s, 1H), 5.63-5.58 (m, 1H), 4.09-3.96 (m, 2H), 3.63 (s, 3H), 2.59 (s, 3H), 2.50-2.40 (m, 1H), 2.15 (d, J=14.40 Hz, 1H), 0.88-0.78 (m, 1H), 0.77-0.69 (m, 1H), 0.67-0.57 (m, 1H), 0.56-0.46 (m, 1H).

### Example 229 and 230

### (S)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide and (R)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide

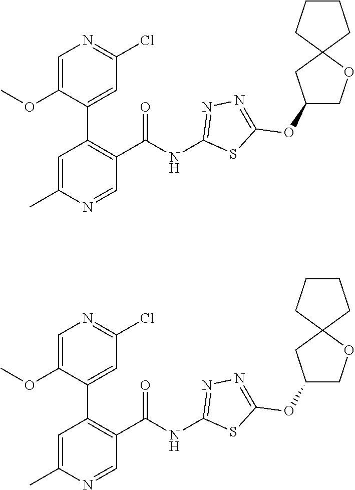 [SMILES: COc1cnc(Cl)cc1-c1cc(C)ncc1C(=O)Nc1nnc(O[C@@H]2COC3(CCCC3)C2)s1 ; COc1cnc(Cl)cc1-c1cc(C)ncc1C(=O)Nc1nnc(O[C@H]2COC3(CCCC3)C2)s1]

### Step-1: S-methyl O-(1-oxaspiro(4.4)nonan-3-yl) carbonodithioate

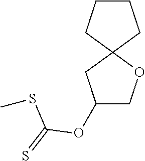

To a stirred solution of 1-oxaspiro(4.4)nonan-3-ol (0.7 g, 4.92 mmol) in Tetrahydrofuran (10 mL) under nitrogen at 0° C. was added sodium hydride (0.394 g, 9.85 mmol) portionwise over 5 min. After addition reaction mixture was stirred at room temperature for 30 min. After 30 min to the above reaction mixture were added carbon disulfide (0.593 mL, 9.85 mmol) and followed by methyl iodide (0.308 mL, 4.92 mmol) at room temperature. The reaction mixture was stirred at room temperature for additional 30 mins. After 30 mins the reaction was quenched with cold water and extracted with ethyl acetate (20 mL×3). The combine organic layer was dried over sodium sulphate, filtered and concentrated under vacuum to afford S-methyl O-(1-oxaspiro(4.4)nonan-3-yl) carbonodithioate (0.95 g, 3.90 mmol, 79% yield) as orange oil.

MS (ESI) calculated for (C10H16O2S2) (M+H)⁺, 233.07. found, 233.0.

### Step-2: O-(1-oxaspiro(4.4)nonan-3-yl) hydrazinecarbothioate

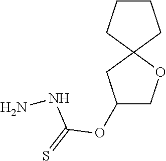 [SMILES: NNC(=S)OC1COC2(CCCC2)C1]

To a stirred solution of S-methyl O-(1-oxaspiro(4.4)nonan-3-yl) carbonodithioate (1.1 g, 4.73 mmol) in Methanol (10 mL) was added hydrazine hydrate (0.237 g, 4.73 mmol) at rt under nitrogen. The reaction mixture was stirred for 2 h at rt. The organic solvent was removed under vacuum. The residue was diluted with water. The aqueous layer was extracted with ethyl acetate (20 mL×3). The combined organic layers were washed with brine, dried over anhydrous sodium sulfate, filtered, and concentrated under vacuum to afford O-(1-oxaspiro(4.4)nonan-3-yl) hydrazinecarbothioate (0.68 g, 2.69 mmol, 56.8% yield) as orange liquid.

MS (ESI) calculated for (C₉H₁₆N₂O₂S) (M+1)⁺, 217.1. found, 217.2.

### Step-3: 5-((1-oxaspiro(4.4)nonan-3-yl)oxy)-1,3,4-thiadiazol-2-amine

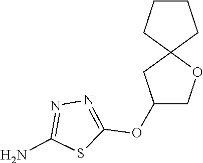 [SMILES: Nc1nnc(OC2COC3(CCCC3)C2)s1]

To a stirred solution of O-(1-oxaspiro(4.4)nonan-3-yl) hydrazinecarbothioate (0.68 g, 3.14 mmol), in Ethanol (10 mL) were added triethylamine (0.438 mL, 3.14 mmol) followed by Cyanogen bromide (0.333 g, 3.14 mmol) at room temperature. The reaction mixture was stirred at room temperature for 1 h. The organic solvent was removed under vacuum. The residue was diluted with water. The aqueous layer was extracted with ethyl acetate (30 mL×3). The combined organic layers were washed with brine, dried over anhydrous sodium sulfate, filtered, and concentrated under vacuum to get the crude product as orange solid. The crude product was pre-absorbed on silica (using 10 mL DCM, 10 g of silica (60-120 mesh), loaded on the biotage 40 g snap, and eluted with 7-9% of methanol in dichloromethane for 30 mins with flow rate 30 ml/min. The appropriate fractions were collected and concentrated under vacuum to afford 5-((1-oxaspiro(4.4)nonan-3-yl)oxy)-1,3,4-thiadiazol-2-amine (0.24 g, 0.733 mmol, 23.31% yield) as yellow solid

MS (ESI) calculated for (C₁₀H₁₅N₃O₂S) (M+1)⁺, 242.1. found, 242.1.

### Step-4: N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide

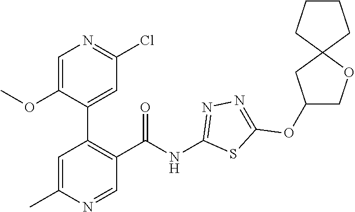 [SMILES: COc1cnc(Cl)cc1-c1cc(C)ncc1C(=O)Nc1nnc(OC2COC3(CCCC3)C2)s1]

To a stirred solution of Intermediate G (200 mg, 0.718 mmol) and 5-((1-oxaspiro(4.4)nonan-3-yl)oxy)-1,3,4-thiadiazol-2-amine (237 mg, 0.718 mmol) in Acetonitrile (2 mL) and N,N-Dimethylformamide (1.2 mL) was added 1-methylimidazole (0.286 mL, 3.59 mmol) and Chloro-N,N,N′,N′-tetramethylformamidinium hexafluorophosphate (221 mg, 0.789 mmol) at rt under nitrogen. The reaction mixture was stirred for 1 h. The reaction mixture was quenched with cold water and extracted with ethyl acetate (10 mL×3). The combined organic layers were washed with brine, dried over anhydrous sodium sulfate, filtered, and concentrated under vacuum to afford the crude product as orange oil.

The crude product was purified by prep-HPLC with the following condition: (Column: YMC C8(20×250) MM 5 MICRON; Mobile Phase A: 10 mM ABC in MQ WATER 80%, Mobile Phase B: acetonitrile 20%; Flow rate: 12 mL/min; Run time: 25 min; (Sample Solvent: THF:ACN; Injection Volume: 200 μL; number of injections: 18) to afford rac-N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide (35 mg, 0.068 mmol, 9.42% yield) as white solid.

### Step-5: (S)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide and (R)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide

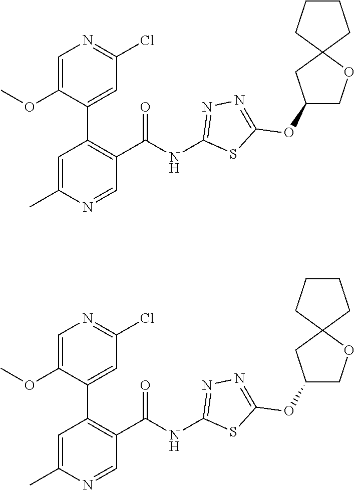 [SMILES: COc1cnc(Cl)cc1-c1cc(C)ncc1C(=O)Nc1nnc(O[C@@H]2COC3(CCCC3)C2)s1 ; COc1cnc(Cl)cc1-c1cc(C)ncc1C(=O)Nc1nnc(O[C@H]2COC3(CCCC3)C2)s1]

Racemic compound N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide (35 mg) was purified by Chiral SFC by using following method with the following condition: Column: YMC Cellulose SC (250*30) mm, 5 μm, Mobile Phase: CO2: 0.5% Isopropyl Amine in IPA (60:40)%, Total Flow: 100 g/min, Back pressure: 100 bar, Wave length: 220 nm, Cycle time: 10 min, a sample was dissolved in 2.0 mL of MeOH/Acetonitrile and injected 800 μl/injection

After SFC purification, the two appropriate fractions were collected.

Fraction-1 was concentrated and lyophilized to afford (S)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide (12 mg) as an off-white solid.

¹H NMR in DMSO-de showed the required protons of the desired compound along with trapped isopropyl amine.

To remove the isopropyl amine, (S)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide (12 mg) was washed MTBE & hexane and lyophillized to afford (S)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide (8 mg, 0.016 mmol, 23.04% yield) as an off-white solid. The absolute stereochemistry was not determined.

(S)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide: MS (ESI) calculated for (C₂₃H₂₄ClN₅O₄S) (M+1)⁺, 502.13. found, 502.1. 1H-NMR (400 MHz, CD₃OD): δ 8.81 (s, 1H), 8.10 (s, 1H), 7.50 (s, 1H), 7.44 (s, 1H), 5.54-5.49 (m, 1H), 4.13 (dd, J=4.8 Hz, 10.8 Hz, 1H), 4.04 (d, J=10.8 Hz, 1H), 3.74 (s, 3H), 2.69 (s, 3H), 2.40 (dd, J=6.4 Hz, 14.4 Hz, 1H), 2.25 (d, J=14.8 Hz, 1H), 1.96-1.81 (m, 2H), 1.80-1.60 (m, 6H).

Fraction-2 was concentrated and lyophilized to afford (R)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide (4 mg, 7.49 μmol, 11.08% yield) as an off-white solid. The absolute stereochemistry was not determined.

(R)—N-(5-((1-oxaspiro(4.4)nonan-3-yl)oxy)-1,3,4-thiadiazol-2-yl)-2′-chloro-5′-methoxy-6-methyl-(4,4′-bipyridine)-3-carboxamide: MS (ESI) calculated for (C₂₃H₂₄ClN₅O₄S) (M+1)⁺, 502.13. found, 502.1. 1H-NMR (400 MHz, CD₃OD): δ 8.81 (s, 1H), 8.10 (s, 1H), 7.50 (s, 1H), 7.43 (s, 1H), 5.54-5.49 (m, 1H), 4.13 (dd, J=4.8 Hz, 10.8 Hz, 1H), 4.04 (d, J=10.8 Hz, 1H), 3.74 (s, 3H), 2.68 (s, 3H), 2.40 (dd, J=6.4 Hz, 14.4 Hz, 1H), 2.25 (d, J=14.4 Hz, 1H), 1.96-1.81 (m, 2H), 1.80-1.60 (m, 6H).

### Example 231

### 4-(5-chloro-2-methoxy-4-(methylsulfinyl)phenyl)-N-(5-methoxy-1,3,4-thiadiazol-2-yl)-6-methylnicotinamide

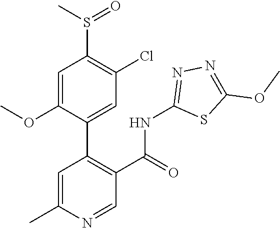

### Step-1: (5-(methoxycarbonyl)-2-methylpyridin-4-yl)boronic Acid

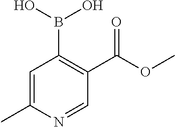

To a stirred solution of methyl 4-chloro-6-methylnicotinate (1 g, 5.39 mmol) in 1,4-Dioxane (10 mL) were added 4,4,4′,4′,5,5,5′,5′-octamethyl-2,2′-bi(1,3,2-dioxaborolane) (2.052 g, 8.08 mmol), and potassium acetate (1.586 g, 16.16 mmol) at room temperature and degassed with nitrogen for 20 minutes. After 20 minutes PdCl2(dppf)-CH2Cl2 adduct (0.308 g, 0.377 mmol) was added and again degassed with nitrogen for 5 minutes. The reaction mixture was stirred at 90° C. for 16 hours. The reaction mixture was cooled to room temperature, inorganic solids were filtered through celite pad and washed with ethyl acetate (200 mL). The filtrate was concentrated under vacuum to afford crude product as brown oil.

The crude product was purified by reverse-phase chromatography with the following condition: (Column: 40 g Reveleris column, C18; Mobile Phase A: 0.1% FA in water, Mobile Phase B: acetonitrile, 0-100%) to afford (5-(methoxycarbonyl)-2-methylpyridin-4-yl)boronic acid (250 mg, 1.281 mmol, 23.77% yield) as an off white solid.

MS (ESI) calculated for (C₈H₁₀BNO₄) (M+1)⁺, 196.08. found, 196.2.

### Step-2: (4-bromo-2-chloro-5-methoxyphenyl)(methyl)sulfane

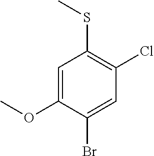

A solution of 4-bromo-2-chloro-5-methoxyaniline (500 mg, 2.114 mmol) in 2N HCl (15.86 mL, 31.7 mmol), was heated to 50° C. for 30 min, then the reaction mixture was cooled to 0° C., and added sodium nitrite (160 mg, 2.326 mmol) in water (10 mL) dropwise. The resulting reaction mixture was stirred for 40 min at 0° C., then sodium thiomethoxide (296 mg, 4.23 mmol) in water (10 mL) was added dropwise at 0° C. The reaction mixture was stirred again for 18 h at room temperature. After completion of the reaction, The reaction mixtures was diluted with 10% NaOH aq. solution (15 mL) and extracted with EtOAc (2×50 mL). The separated organic layer was dried over anhydrous Na2SO4 and concentrated under vacuum to afford crude product as light yellow oil.

The crude product was pre-absorbed on silica (using 5 mL DCM, 3 g of silica (60-120 mesh), loaded on the pre-packed Orochem 40 g column and eluted at 0-30% of ethyl acetate in PET ether for 45 minutes with flow rate 20 ml/min. The appropriate fractions were collected and concentrated reduced pressure to afford (4-bromo-2-chloro-5-ethoxyphenyl)(methyl)sulfane (420 mg, 1.428 mmol, 67.6% yield) as yellow solid.

GCMS calculated for (C₈H₈BrClOS) (M)⁺, 267.91. found, (m/z) 267.9.

### Step-3: 1-bromo-5-chloro-2-methoxy-4-(methylsulfinyl)benzene

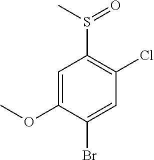

To a stirred solution of (4-bromo-2-chloro-5-methoxyphenyl)(methyl)sulfane (500 mg, 1.869 mmol) in Dichloromethane (10 mL) was added mCPBA (355 mg, 2.056 mmol) portion wise at 0° C., under nitrogen atmosphere. The resulting mixture was stirred at room temperature for 16 h. The reaction mixtures was diluted with 10% Na₂CO₃ aq. solution (30 mL) and extracted with EtOAc (2×50 mL), washed with brine. The organic layer was separated and dried over anhydrous Na₂SO₄, concentrated under vacuum to afford crude product as light yellow oil. The crude product was pre-absorbed on silica (using 5 mL DCM, 2 g of silica (60-120 mesh), loaded on the pre-packed Orochem 40 g column and eluted at 0-60% of ethyl acetate in PET ether for 60 minutes with flow rate 15 ml/min. The appropriate fractions were collected and concentrated reduced pressure to afford 1-bromo-5-chloro-2-methoxy-4-(methylsulfinyl)benzene (200 mg, 0.684 mmol, 36.6% yield) as brown solid.

MS (ESI) calculated for (C₈H₈BrClO₂S) (M+1)⁺, 282.92. found, 282.8.

### Step-4: methyl 4-(5-chloro-2-methoxy-4-(methylsulfinyl)phenyl)-6-methylnicotinate

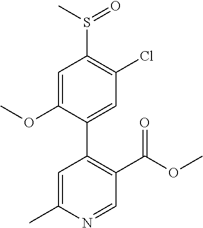

To a stirred solution of 1-bromo-5-chloro-2-methoxy-4-(methylsulfinyl)benzene (200 mg, 0.705 mmol) in 1,4-Dioxane (10 mL) were added methyl (5-(methoxycarbonyl)-2-methylpyridin-4-yl)boronic acid (275 mg, 1.411 mmol) and potassium carbonate (292 mg, 2.116 mmol) at room temperature and degassed with nitrogen for 10 minutes. To the resulting reaction mixture was added PdCl2(dppf) (103 mg, 0.141 mmol). The reaction mixture was stirred at 100° C. for 16 hours. After completion of the reaction, the reaction mixture was cooled to room temperature, inorganic solids were filtered through celite pad and washed with ethyl acetate (100 mL). The filtrate was washed with water (50 mL), dried over sodium sulphate, filtered and concentrated under reduced pressure to afford crude product as light brown oil.

The crude product was pre-absorbed on silica (using 10 mL DCM, 3 g of silica (60-120 mesh), loaded on the pre-pack orochem 40 g column and eluted at 0-80% of ethyla acetate in PET ether for 45 minutes with flow rate 15 ml/min. The appropriate fractions were collected and concentrated reduced pressure to afford methyl 4-(5-chloro-2-methoxy-4-(methylsulfinyl)phenyl)-6-methylnicotinate (120 mg, 0.326 mmol, 46.2% yield) as an light brown oil

MS (ESI) calculated for (C₁₆H₁₆ClNO₄S) (M+1)⁺, 354.06. found, 354.0.

### Step-5: 4-(5-chloro-2-methoxy-4-(methylsulfinyl)phenyl)-6-methylnicotinic Acid

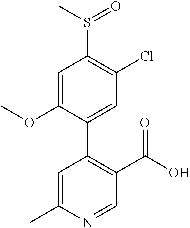 [SMILES: COc1cc(S(C)=O)c(Cl)cc1-c1cc(C)ncc1C(=O)O]

To a stirred solution of methyl 4-(5-chloro-2-methoxy-4-(methylsulfinyl)phenyl)-6-methylnicotinate (120 mg, 0.339 mmol) in a mixture of Tetrahydrofuran (2 mL), Methanol (2 mL) and Water (2 mL) was added lithiumhydroxide·monohydrate (40.6 mg, 1.696 mmol) at room temperature. The reaction mixture was stirred at room temperature for 2.5 hours. After completion of the reaction, the reaction mixture was concentrated under vacuum. The resulting residue diluted with water (3 mL), the aqueous layer was acidified with 1.5 N HCl upto pH-6 and extracted with ethyl acetate (3×20 mL). The combined organic layer dried over anhydrous sodium sulphate, filtered concentrated under reduced pressure to afford 4-(5-chloro-2-methoxy-4-(methylsulfinyl)phenyl)-6-methylnicotinic acid (60 mg, 0.155 mmol, 45.8% yield) as light brown solid.

MS (ESI) calculated for (C₁₅H₁₄ClNO₄S) (M+1)⁺, 340.04. found, 340.0.

### Step-6: 4-(5-chloro-2-methoxy-4-(methylsulfinyl)phenyl)-N-(5-methoxy-1,3,4-thiadiazol-2-yl)-6-methylnicotinamide

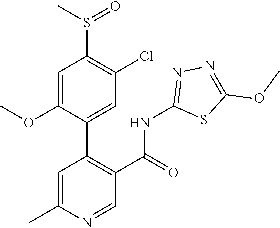

To a stirred solution of 4-(5-chloro-2-methoxy-4-(methylsulfinyl)phenyl)-6-methylnicotinic acid (30 mg, 0.088 mmol), in Acetonitrile (2 mL) were added Chloro-N,N,N′,N′-tetramethylformamidinium hexafluorophosphate (27.2 mg, 0.097 mmol), and 1-methylimidazole (0.028 mL, 0.353 mmol) the reaction mixture was stirred at room temperature for 30 min under nitrogen, then 5-methoxy-1,3,4-thiadiazol-2-amine (13.90 mg, 0.106 mmol)) was added. The reaction mixture was stirred at same temperature for 16 h. The reaction mixture was diluted with water, extracted with ethyl acetate (2×10 mL). The combined organic layer dried over anhydrous sodium sulphate, filtered concentrated under vacuum to afford crude product as brown oil.

The crude product was purified by prep-HPLC with the following condition: (Column: X-select C18 (19*150) MM 5 MICRON; Mobile Phase A: 10 mM ABC in Milli Q Water 90%, Mobile Phase B: Acetonitrile 10%; Flow rate: 8 mL/min; Run time: 18 min; (Sample Solvent: THF:ACN; Injection Volume: 200 μL; number of injections: 12) to afford 4-(5-chloro-2-methoxy-4-(methylsulfinyl)phenyl)-N-(5-methoxy-1,3,4-thiadiazol-2-yl)-6-methylnicotinamide (4.5 mg, 9.06 μmol, 10.26% yield) as a white solid. MS (ESI) calculated for (C₁₈H₁₇ClN₄O₄S₂) (M+1)⁺, 453.05. found, 453.0.

1H-NMR (400 MHz, DMSO-d⁶): δ 12.85 (s, 1H), 8.77 (s, 1H), 7.60 (s, 1H), 7.38 (s, 1H), 7.34 (s, 1H), 4.06 (s, 3H), 3.61 (s, 3H), 2.86 (s, 3H), 2.58 (s, 3H).

### Example 234

### 1-(2-chloro-5-methoxypyridin-4-yl)-N-(5-methoxy-1,3,4-thiadiazol-2-yl)-1H-1,2,3-triazole-4-carboxamide

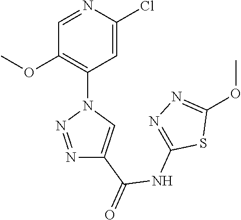

### Step-1: 4-azido-2-chloro-5-methoxypyridine

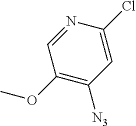

To a degassed solution of (2-chloro-5-methoxypyridin-4-yl)boronic acid (4.0 g, 21.35 mmol) in Methanol (5 mL) were added CuSO₄ (171.0 mg, 1.06 mmol) and sodium azide (0.7 g, 10.67 mmol) at 23° C. The resulting solution was stirred at 80° C. for 1 h. The reaction mixture was quenched by the addition of water and extracted with dichloromethane. The combined organic layers were washed with brine, dried over anhydrous sodium sulfate, filtered, and concentrated under vacuum. The resulting residue was dissolved in DCM (5 mL) and purified by Combi Flash (Biotage Isolera Prime) which applied to a 40 g silica gel column that was eluted with 0˜80% ethyl acetate in petroleum ether within 25 min to afford 4-azido-2-chloro-5-methoxypyridine (570 mg, 13%) as a white solid. MS (ESI) calc'd for (C₆H₅ClN₄O) (M+1)*, 185.0. found 185.0.

### Step-2: ethyl 1-(2-chloro-5-methoxypyridin-4-yl)-1H-1,2,3-triazole-4-carboxylate

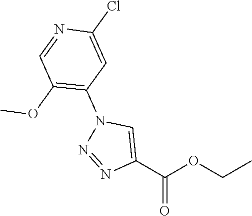 [SMILES: CCOC(=O)c1cn(-c2cc(Cl)ncc2OC)nn1]

To a stirred solution of 4-azido-2-chloro-5-methoxypyridine (570.0 mg, 3.09 mmol) in Toluene (1 mL) was added ethyl propiolate (218.0 mg, 2.22 mmol) at 23° C. The resulting solution was stirred at 80° C. for 1 h. The solvents were removed under vacuum to afford ethyl 1-(2-chloro-5-methoxypyridin-4-yl)-1H-1,2,3-triazole-4-carboxylate (800.0 mg, crude) as a yellow solid. MS (ESI) calc'd for (C₁₁H₁₁ClN₄O₃) (M+1)⁺, 283.1. found, 283.1.

### Step-3: 1-(2-chloro-5-methoxypyridin-4-yl)-1H-1,2,3-triazole-4-carboxylic Acid

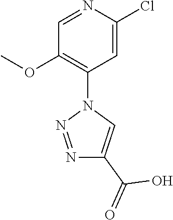 [SMILES: COc1cnc(Cl)cc1-n1cc(C(=O)O)nn1]

To a stirred solution of ethyl 1-(2-chloro-5-methoxypyridin-4-yl)-1H-1,2,3-triazole-4-carboxylate (400.0 mg, 1.41 mmol) in THF (5 mL) and Water (5 mL) was added LiOH (67.8 mg, 2.83 mmol). The resulting solution was stirred at 23° C. for 1 h. The organic solvent was removed under vacuum. The aqueous layer was acidified with citric acid to pH ˜3. The resulting residue was dissolved in DMF (1 mL) which was applied to a 40 g C18 column and purified by Combi Flash (Biotage Isolera Prime), eluted with 5˜50% acetonitrile in water within 20 min to afford 1-(2-chloro-5-methoxypyridin-4-yl)-1H-1,2,3-triazole-4-carboxylic acid (160.0 mg, 44%) as a white solid MS (ESI) calc'd for (C₉H₇ClN₄O₃) (M+1)⁺, 255.0. found, 255.0.

### Step-4: 1-(2-chloro-5-methoxypyridin-4-yl)-N-(5-methoxy-1,3,4-thiadiazol-2-yl)-1H-1,2,3-triazole-4-carboxamide

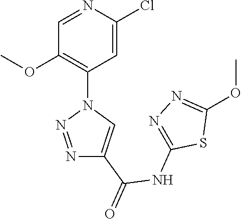

To a stirred solution of 1-(2-chloro-5-methoxypyridin-4-yl)-1H-1,2,3-triazole-4-carboxylic acid (100 mg, 0.39 mmol), 5-methoxy-1,3,4-thiadiazol-2-amine (51.5 mg, 0.39 mmol) in Acetonitrile (0.2 mL) were added 1-methylimidazole (161.0 mg, 1.96 mmol) and TCFH (110.0 mg, 0.39 mmol). The resulting mixture was then stirred at 23° C. for 1 h. The suspension was filtered. The filter cake was collected and dried under vacuum to afford 1-(2-chloro-5-methoxypyridin-4-yl)-N-(5-methoxy-1,3,4-thiadiazol-2-yl)-1H-1,2,3-triazole-4-carboxamide (69.2 mg, 47%) as a white solid. MS (ESI) calc'd for (C₁₂H₁₀ClN₇O₃S) (M+1)⁺, 368.0. found 368.0. ¹H NMR (400 MHz, DMSO-d₆) δ 13.02 (s, 1H), 9.42 (s, 1H), 8.59 (s, 1H), 8.06 (s, 1H), 4.10 (s, 6H).

### Example 235

### 1-(2-chloro-5-methoxypyridin-4-yl)-N-(5-methoxy-1,3,4-thiadiazol-2-yl)-1H-pyrazole-3-carboxamide

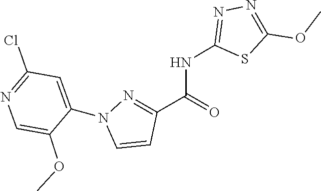 [SMILES: COc1nnc(NC(=O)c2ccn(-c3cc(Cl)ncc3OC)n2)s1]

### Step-1: 2-chloro-4-iodo-5-methoxypyridine

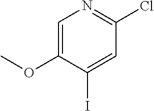

To a degassed solution of 6-chloro-4-iodopyridin-3-ol (5.0 g, 19.57 mmol) in N,N-Dimethylformamide (20 mL) was added NaH (939.0 mg, 23.49 mmol, 60%) in portions at 0° C. and stirred at 0° C. for 40 min. To the above solution was added MeI (4.1 g, 29.40 mmol) at 0° C. under nitrogen. The resulting solution was then stirred at 25 º C for 16 h. The reaction mixture was quenched by the addition of water and extracted with ethyl acetate. The combined organic layers were washed with brine, dried over anhydrous sodium sulfate, filtered, and concentrated under vacuum. The resulting residue was purified by Combi Flash (Biotage Isolera Prime) which applied to 120 g silica gel column and eluted with 0˜50% ethyl acetate in petroleum ether within 45 min to afford 2-chloro-4-iodo-5-methoxypyridine (3.1 g, 51%) as a white solid. MS (ESI) calc'd for (C₆H₅ClINO) (M+1)⁺, 269.9. found 269.9.

### Step-2: ethyl 1-(2-chloro-5-methoxypyridin-4-yl)-1H-pyrazole-3-carboxylate

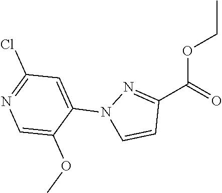 [SMILES: CCOC(=O)c1ccn(-c2cc(Cl)ncc2OC)n1]

To a degassed solution of 2-chloro-4-iodo-5-methoxypyridine (1.0 g, 3.71 mmol) in N,N-Dimethylformamide (10 mL) were added Cs₂CO₃ (2.4 g, 7.42 mmol), ethyl 1H-pyrazole-5-carboxylate (728.0 mg, 5.20 mmol) and copper(I) iodide (141.0 mg, 0.74 mmol). The resulting solution was stirred at 25° C. for 30 min under nitrogen, then the solution was heated to 100° C. stirred for additional 1 h. The suspension was filtered. The filtrate was collected and concentrated under vacuum. The mixture was dissolved in DMF (3 mL) which was applied to a 80 g C18 column and purified by Combi Flash (Biotage Isolera Prime), eluted with 5˜60% acetonitrile in water within 25 min to afford ethyl 1-(2-chloro-5-methoxypyridin-4-yl)-1H-pyrazole-3-carboxylate (690.1 mg, 59%) as a white solid. MS (ESI) calc'd for (C₁₂H₁₂ClN₃O3) (M+1)⁺, 282.1. found, 282.1.

### Step-3: 1-(2-chloro-5-methoxypyridin-4-yl)-1H-pyrazole-3-carboxylic Acid

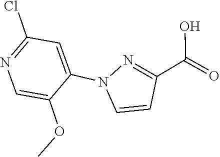 [SMILES: COc1cnc(Cl)cc1-n1ccc(C(=O)O)n1]

To a solution of ethyl 1-(2-chloro-5-methoxypyridin-4-yl)-1H-pyrazole-3-carboxylate (300.0 mg, 1.07 mmol) in Tetrahydrofuran (3 mL) and Water (1 mL) was added LiOH (25.5 mg, 1.06 mmol). The resulting solution was stirred at 25° C. for 2 h. The organic solvent was removed under vacuum. The aqueous layer was acidified with Formic acid to pH ˜3 and extracted with ethyl acetate. The combined organic layers were washed with brine, dried over anhydrous sodium sulfate, filtered, and concentrated under vacuum The mixture was dissolved in DMF (2 mL) which was applied to a 40 g C18 column and purified by Combi Flash (Biotage Isolera Prime), eluted with 5˜50% acetonitrile in water within 20 min to afford 1-(2-chloro-5-methoxypyridin-4-yl)-1H-pyrazole-3-carboxylic acid (270.2 mg, 90%) as a white solid. MS (ESI) calc'd for (C₁₀H₈ClN₃O3) (M+1)⁺, 254.0.1. found, 254.0.

### Step-4: 1-(2-chloro-5-methoxypyridin-4-yl)-N-(5-methoxy-1,3,4-thiadiazol-2-yl)-1H-pyrazole-3-carboxamide

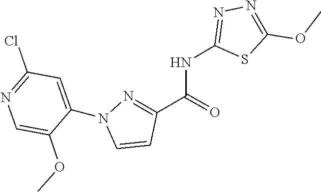 [SMILES: COc1nnc(NC(=O)c2ccn(-c3cc(Cl)ncc3OC)n2)s1]

To a solution of 1-(2-chloro-5-methoxypyridin-4-yl)-1H-pyrazole-3-carboxylic acid (100.0 mg, 0.39 mmol) in Acetonitrile (1 mL) were added 5-methoxy-1,3,4-thiadiazol-2-amine (51.7 mg, 0.39 mmol) and 1-methyl-1H-imidazole (162.2 mg, 1.97 mmol). To the above was added a solution of TCFH (111.1 mg, 0.39 mmol) in Acetonitrile (1 mL) at 25° C. The resulting solution was stirred at 25° C. for 2 h under nitrogen. The mixture was dissolved in DMF (2 mL) which was applied to a 40 g C18 column and purified by Combi Flash (Biotage Isolera Prime), eluted with 5˜50% acetonitrile in water within 20 min to afford 1-(2-chloro-5-methoxypyridin-4-yl)-N-(5-methoxy-1,3,4-thiadiazol-2-yl)-1H-pyrazole-3-carboxamide (53.1 mg, 36%) as a white solid. MS (ESI) calc'd for (C₁₃H₁₁ClN₆O₃S) (M+1)⁺, 367.0. found, 367.1. ¹H NMR (400 MHz, DMSO-d₆) δ 12.82 (s, 1H), 8.67 (d, J=2.8 Hz, 1H), 8.47 (s, 1H), 8.36 (s, 1H), 7.15 (d, J=2.4 Hz, 1H), 4.11 (s, 3H), 4.08 (s, 3H).

### Example 242

### 2′-chloro-5′,6-dimethoxy-N-(5-methoxy-1,3,4-thiadiazol-2-yl)-[3,4′-bipyridine]-4-carboxamide

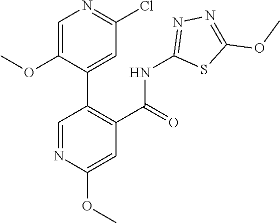

### Step-1: Synthesis of methyl 2′-chloro-5′,6-dimethoxy-[3,4′-bipyridine]-4-carboxylate

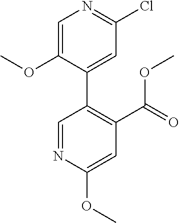 [SMILES: COC(=O)c1cc(OC)ncc1-c1cc(Cl)ncc1OC]

To a stirred solution of methyl 5-bromo-2-methoxyisonicotinate (1 g, 4.06 mmol) in dioxane (15 ml) and H₂O (1.5 ml) was added potassium carbonate (0.674 g, 4.88 mmol), Bis(diphenylphosphino)ferrocene]dichloropalladium(II) (0.178 g, 0.244 mmol) and (2-chloro-5-methoxypyridin-4-yl)boronic acid (0.838 g, 4.47 mmol). The reaction mixture was stirred at 85° C. for 8 hours. After completion, the reaction mixture was cooled to room temperature, diluted with water (100 mL) and extracted with ethyl acetate (3×100 mL). The combined organic layer was dried over sodium sulfate, filtered and concentrated under reduced pressure to afford crude product as a brown solid.

The crude product was pre-absorbed on silica, loaded on the pre-packed Biotage (12 g) column and eluted with 20% of ethyl acetate in pet ether for 20 minutes with flow rate 30 ml/min. The appropriate fractions were collected and concentrated vacuum to afford methyl 2′-chloro-5′,6-dimethoxy-[3,4′-bipyridine]-4-carboxylate (680 mg, 2.203 mmol, 54.2% yield) as a white solid.

MS (ESI) calculated for (C₁₄H₁₃ClN₂O₄) (M+1)⁺, 309.07. found, 309.0.

### Step-2: Synthesis of 2′-chloro-5′,6-dimethoxy-N-(5-methoxy-1,3,4-thiadiazol-2-yl)-[3,4′-bipyridine]-4-carboxamide

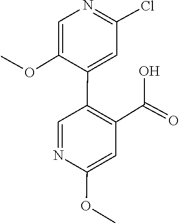 [SMILES: COc1cc(C(=O)O)c(-c2cc(Cl)ncc2OC)cn1]

To a stirred solution of methyl 2′-chloro-5′,6-dimethoxy-[3,4′-bipyridine]-4-carboxylate (680 mg, 2.203 mmol) in tetrahydrofuran (6 mL), methanol (3 mL) and water (3 mL) was added lithium hydroxide monohydrate (92 mg, 2.203 mmol) at 0° C. The reaction mixture was stirred at room temperature for 2 hours. After completion, reaction mixture was concentrated under vacuum, residue was acidified with 1.5 N HCl up to pH-3 to give off-white precipitate. The obtained precipitate was filtered and dried under vacuum to afford 2′-chloro-5′,6-dimethoxy-[3,4′-bipyridine]-4-carboxylic acid (440 mg, 1.380 mmol, 62.6% yield) as a white solid.

MS (ESI) calculated for (C₁₃H₁₁ClN₂O₄) (M+1)⁺, 295.05. found, 295.0.

### Step-3: Synthesis of 2′-chloro-5′,6-dimethoxy-N-(5-methoxy-1,3,4-thiadiazol-2-yl)-[3,4′-bipyridine]-4-carboxamide

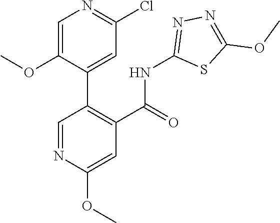

To a stirred solution of 2′-chloro-5′,6-dimethoxy-[3,4′-bipyridine]-4-carboxylic acid (50 mg, 0.170 mmol) in acetonitrile (3 mL) and N,N-dimethylformamide (0.6 mL) were added chloro-N,N,N′,N′-tetramethylformamidinium hexafluorophosphate (52.4 mg, 0.187 mmol), 1-methyl-1H-imidazole (41.8 mg, 0.509 mmol) and 5-methoxy-1,3,4-thiadiazol-2-amine (22.25 mg, 0.170 mmol) at room temperature. The reaction mixture was stirred at room temperature for 8 hours. After completion, the reaction mixture was quenched with cold water (10 mL) and extracted by ethyl acetate (2×20 mL). The combined organic layer dried over anhydrous sodium sulfate, filtered, concentrated under vacuum to afford crude product as a yellow semi solid.

The crude product (200 mg) of this batch was mixed crude product (170 mg) of another batch and combined two batches were purified together using prep-HPLC.

The crude product was purified by prep-HPLC with the following condition: (X-select (19*250 mm) 5 MICRON; Mobile Phase A: 10 mM ABC in Milli Q Water 80%, Mobile Phase B: Acetonitrile 20% Flow rate: 12 mL/min; Run time: 17 min; (Sample Solvent: THF:ACN; Injection Volume: 400 μL; number of injections: 25) to afford 2′-chloro-5′,6-dimethoxy-N-(5-methoxy-1,3,4-thiadiazol-2-yl)-[3,4′-bipyridine]-4-carboxamide (52 mg, 0.125 mmol, 73.4% yield) as an off-white solid.

MS (ESI) calculated for (C₁₆H₁₄ClN₅O₄S) (M+1)⁺, 408.06. found, 408.0.

1H-NMR (400 MHz, DMSO-d⁶): δ 12.98 (brs, 1H), 8.30 (s, 1H), 8.11 (s, 1H), 7.53 (s, 1H), 7.20 (s, 1H), 4.08 (s, 3H), 3.97 (s, 3H), 3.60 (s, 3H)

### Example 243

### 3′-fluoro-N-(5-(((3R,3aS,6aR)-hexahydrofuro[2,3-b]furan-3-yl)oxy)-1,3,4-thiadiazol-2-yl)-5′-methoxy-2′,6-dimethyl-[4,4′-bipyridine]-3-carboxamide

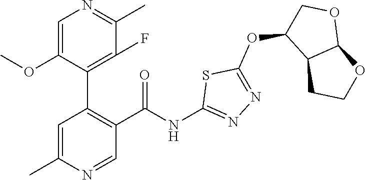 [SMILES: COc1cnc(C)c(F)c1-c1cc(C)ncc1C(=O)Nc1nnc(O[C@H]2CO[C@H]3OCC[C@H]32)s1]

### Step-1. O-((3R,3aS,6aR)-hexahydrofuro[2,3-b]furan-3-yl)S-methyl Carbonodithioate

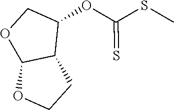

To a solution of ((3R,3aS,6aR)-hexahydrofuro[2,3-b]furan-3-ol (3.7 g, 28.4 mmol) in tetrahydrofuran (THF) (100 mL) was added NaH (60%, 1.478 g, 37.0 mmol) at 0° C. The mixture was stirred at 0° C. for 5 min before the dropwise addition of carbon disulfide (2.60 mL, 31.3 mmol) followed by methyl iodide (1.955 mL, 31.3 mmol). The mixture was allowed to stir for 10 min before quenching with saturated aq. NH₄Cl (5 mL) and brine (10 mL). The mixture was diluted with EtOAc (30 mL) and the layers were separated. The aq. layer was washed with EtOAc (2×20 mL). The combined organic layer was dried over MgSO₄, filtered and the filtrate was concentrated under vacuum. The residue was purified by silica gel (0-100% EtOAc/heptane, 80 g column) to give the desired product as a yellow oil. LCMS (ES)=221.2 [M+H]^{+ 1}H NMR (400 MHz, CHLOROFORM-d) δ 5.91 (dt, J=8.3, 6.4 Hz, 1H), 5.76 (d, J=4.9 Hz, 1H), 4.17 (dd, J=10.3, 6.4 Hz, 1H), 4.04-3.88 (m, 3H), 3.28-3.17 (m, 1H), 2.59 (s, 3H), 2.08-2.00 (m, 1H), 1.98-1.87 (m, 1H).

### Step-2. O-((3R,3aS,6aR)-hexahydrofuro[2,3-b]furan-3-yl) hydrazinecarbothioate

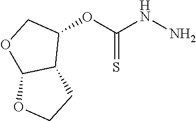 [SMILES: NNC(=S)O[C@H]1CO[C@H]2OCC[C@H]21]

To a solution of (O-((3R,3aS,6aR)-hexahydrofuro[2,3-b]furan-3-yl)S-methyl carbonodithioate (5.8 g, 26.3 mmol) in methanol (100 mL) at 0° C. was added hydrazine hydrate solution (1.136 mL, 29.0 mmol) dropwise. The mixture was stirred at 0° C. for 2 h. Upon completion, the reaction was concentrated under vacuum. The residue was azeotroped in a mixture of MeOH/toluene (1 mL/10 mL) to give the crude product O-((3R,3aS,6aR)-hexahydrofuro[2,3-b]furan-3-yl) hydrazinecarbothioate (5.38 g, 26.3 mmol, 100% yield). LCMS (ES)=205.2 [M+H]+.

### Step-3. 5-(((3R,3aS,6aR)-hexahydrofuro[2,3-b]furan-3-yl)oxy)-1,3,4-thiadiazol-2-amine

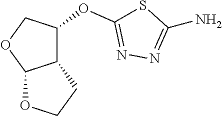 [SMILES: Nc1nnc(O[C@H]2CO[C@H]3OCC[C@H]32)s1]

To a solution of (O-((3R,3aS,6aR)-hexahydrofuro[2,3-b]furan-3-yl) hydrazinecarbothioate (5.3 g, 25.9 mmol) in methanol (100 mL) at 0° C. was added triethylamine (7.96 mL, 57.1 mmol) followed by cyanic bromide (3M in DCM) (10.38 mL, 31.1 mmol) dropwise. The mixture was stirred at 0° C. for 2 h. Upon completion, the reaction was concentrated under vacuum and the residue was purified by silicagel chromatography (0-5% MeOH/DCM, 80 g column) to give the desired product as a brown solid. LCMS (ES)=230.2 [M+H]⁺. ¹H NMR (400 MHz, DMSO-d₆) δ 6.90 (s, 2H), 5.64 (d, J=5.4 Hz, 1H), 5.24 (dt, J=8.3, 6.4 Hz, 1H), 4.06 (dd, J=9.8, 6.4 Hz, 1H), 3.87 (td, J=8.1, 2.4 Hz, 1H), 3.79-3.70 (m, 2H), 3.21-3.10 (m, 1H), 2.04-1.93 (m, 1H), 1.88-1.73 (m, 1H).

### Step-4. 3′-fluoro-N-(5-(((3R,3aS,6aR)-hexahydrofuro[2,3-b]furan-3-yl)oxy)-1,3,4-thiadiazol-2-yl)-5′-methoxy-2′,6-dimethyl-[4,4′-bipyridine]-3-carboxamide

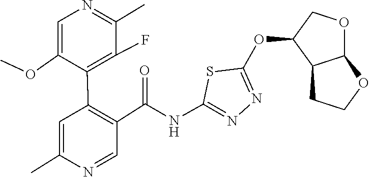 [SMILES: COc1cnc(C)c(F)c1-c1cc(C)ncc1C(=O)Nc1nnc(O[C@H]2CO[C@H]3OCC[C@H]32)s1]

To a solution of 3′-fluoro-5′-methoxy-2′,6-dimethyl-[4,4′-bipyridine]-3-carboxylic acid (100 mg, 0.362 mmol, Example 61, Step 2) in acetonitrile (10 mL) at 23° C. were added N-(chloro(dimethylamino)methylene)-N-methylmethanaminium hexafluorophosphate(V) (112 mg, 0.398 mmol) and 1-methyl-1H-imidazole (62.4 mg, 0.760 mmol). The reaction was allowed to stir for 5 min before adding 5-(((3R,3aS,6aR)-hexahydrofuro[2,3-b]furan-3-yl)oxy)-1,3,4-thiadiazol-2-amine (91 mg, 0.398 mmol) after which the reaction was allowed to stir at 23° C. for 1.5 h. The reaction was concentrated under vacuum, taken up in EtOAc and washed with saturated aq. NH4Cl. The EtOAc layer was dried over MgSO₄, filtered and the filtrate was concentrated under vacuum. The residue was purified by silica gel column (0-100% (3:1)(EtOAc/EtOH)/heptane, 40 g column) to give the desired product 3′-fluoro-N-(5-(((3R,3aS,6aR)-hexahydrofuro[2,3-b]furan-3-yl)oxy)-1,3,4-thiadiazol-2-yl)-5′-methoxy-2′,6-dimethyl-[4,4′-bipyridine]-3-carboxamide (122 mg, 0.250 mmol, 69.1% yield) as a light brown solid. MS (ESI) calc'd for (C₂₂H₂₂FN₅O₅S) (M+1)⁺, 488.0. found 488.3. ¹H NMR (400 MHz, METHANOL-d4) δ 8.89 (s, 1H), 8.09 (s, 1H), 7.46 (s, 1H), 5.74 (d, J=5.4 Hz, 1H), 5.46 (dt, J=7.9, 6.1 Hz, 1H), 4.20 (dd, J=9.8, 6.4 Hz, 1H), 4.01-3.88 (m, 3H), 3.77 (d, J=1.5 Hz, 3H), 3.32-3.25 (m, 1H), 2.68 (s, 3H), 2.50 (d, J=2.9 Hz, 3H), 2.23-2.08 (m, 1H), 2.03-1.84 (m, 1H).

### Example 262

### 2′-chloro-N-(5-(((3R,3aS,6aR)-hexahydrofuro[2,3-b]furan-3-yl)oxy)-1,3,4-thiadiazol-2-yl)-5′-methoxy-6-methyl-[4,4′-bipyridine]-3-carboxamide

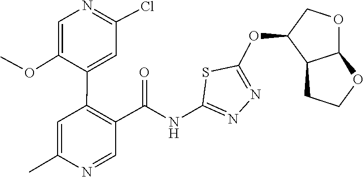 [SMILES: COc1cnc(Cl)cc1-c1cc(C)ncc1C(=O)Nc1nnc(O[C@H]2CO[C@H]3OCC[C@H]32)s1]

To a solution of Intermediate G (498 mg, 1.788 mmol), 5-(((3R,3aS,6aR)-hexahydrofuro[2,3-b]furan-3-yl)oxy)-1,3,4-thiadiazol-2-amine (410 mg, 1.788 mmol), and 1-methyl-1H-imidazole (147 mg, 1.788 mmol, Example 243, Step 3) in acetonitrile (10 mL) at 0° C. was added N-(chloro(dimethylamino)methylene)-N-methylmethanaminium hexafluorophosphate(V) (552 mg, 1.967 mmol). The mixture was allowed to stir at 0° C. for 1 h. The reaction mixture was concentrated under vacuum and purified by silica gel column (0-100% (3:1)(EtOAc/EtOH)/Heptane, 40 g column, 20 mins) to give the desired product 2′-chloro-N-(5-(((3R,3aS,6aR)-hexahydrofuro[2,3-b]furan-3-yl)oxy)-1,3,4-thiadiazol-2-yl)-5′-methoxy-6-methyl-[4,4′-bipyridine]-3-carboxamide (326 mg, 0.665 mmol, 37.2% yield) as a light pink solid. MS (ESI) calc'd for (C₂₁H₂₀ClN₅O₅S) (M+1)⁺, 490.0. found 490.2. ¹H NMR (400 MHz, CHLOROFORM-d) δ 12.98-12.59 (m, 1H), 9.08 (s, 1H), 8.05 (s, 1H), 7.26 (s, 1H), 7.21 (s, 1H), 5.81 (d, J=5.4 Hz, 1H), 5.42-5.33 (m, 1H), 4.21 (dd, J=9.8, 6.8 Hz, 1H), 4.08-3.93 (m, 2H), 3.88 (dd, J=9.8, 7.3 Hz, 1H), 3.74 (s, 3H), 3.34-3.23 (m, 1H), 2.70 (s, 3H), 2.23-2.11 (m, 1H), 2.03-1.87 (m, 1H).

### Example 270

### 2′-chloro-6-ethyl-5′-methoxy-N-(5-methoxy-1,3,4-thiadiazol-2-yl)-[3,4′-bipyridine]-4-carboxamide

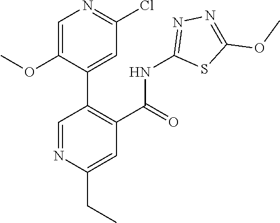

### Step-1: Synthesis of methyl 5-chloro-2-vinylisonicotinate

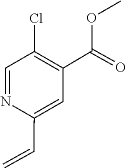

A solution of methyl 2,5-dichloroisonicotinate (1 g, 4.85 mmol), 4,4,5,5-tetramethyl-2-vinyl-1,3,2-dioxaborolane (0.822 g, 5.34 mmol) and potassium carbonate (1.342 g, 9.71 mmol) in acetonitrile (15 mL) and methanol (2 mL) was degassed with nitrogen for 15 minutes followed by addition of palladium (II) acetate (54 mg, 0.243 mmol) and triphenylphosphine (0.127 g, 0.485 mmol). The reaction mixture was heated to 40° C. and stirred for 16 hours. The reaction mixture was quenched with water and extracted with ethyl acetate (2×200 mL). The combine organic layer was dried over sodium sulfate, filtered and concentrated to give crude product as yellow liquid.

The crude was pre-absorbed on silica, loaded on pre-packed biotage column (25 g), and then eluted 2-10% of ethyl acetate in pet ether for 40 mins with flow rate 25 ml/min. The fractions were collected and concentrated under reduced pressure to afford methyl 5-chloro-2-vinylisonicotinate (730 mg, 3.66 mmol, 75% yield) as colourless liquid.

MS (ESI) calculated for (C₉H₈ClNO₂) (M+1)⁺, 198.03. found, 198.0.

### Step-2: Synthesis of methyl 5-chloro-2-methylisonicotinate

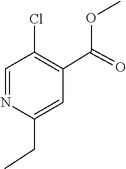

To a solution of methyl 5-chloro-2-vinylisonicotinate (0.62 g, 3.14 mmol) in ethyl acetate (25 mL) stirred under nitrogen at room temp was added palladium, (5 wt % on calcium carbonate, poisoned with lead, 1.669 g, 0.784 mmol) in one charge. The reaction mixture was stirred at room temperature under hydrogen atmosphere for 2 hours. After completion, the reaction mixture was filtered through celite bed and washed with ethyl acetate (100 mL). The filtrate was concentrated under reduced pressure to give crude product as brown liquid.

The crude product was pre-absorbed on silica, loaded on the pre-packed Orochem 25 g column and eluted at 5-15% of ethyl acetate in PET ether for 20 minutes with flow rate 25 ml/min. The appropriate fractions were collected and concentrated under reduced pressure to afford methyl 5-chloro-2-ethylisonicotinate (530 mg, 2.56 mmol, 81% yield) as brown oil.

LCMS calculated for (C₉H₁₀ClNO₂) (M+1)⁺, 200.05. found, 200.2.

### Step-3: Synthesis of methyl 2′-chloro-6-ethyl-5′-methoxy-[3,4′-bipyridine]-4-carboxylate

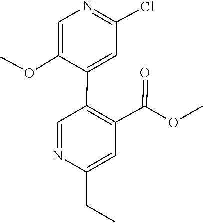

To a solution of methyl 5-chloro-2-ethylisonicotinate (530 mg, 2.65 mmol) and (2-chloro-5-methoxypyridin-4-yl)boronic acid (497 mg, 2.65 mmol) in 1,4-Dioxane (15 mL) was added a solution of potassium carbonate (734 mg, 5.31 mmol) in water (3.00 mL) in one charge under nitrogen at room temperature. The reaction mixture was degassed for 10 min with nitrogen followed by addition of [1,1′-Bis(di-tert-butylphosphino)ferrocene]dichloropalladium(II) (173 mg, 0.265 mmol) at room temperature. The resulting reaction mixture was stirred at 80° C. for 16 hours. After completion, the reaction mixture was cooled to room temperature and filtered through celite bed, washed with ethyl acetate (25 mL). The filtrate was washed with water (20 mL) and brine solution. The organic layer was dried over anhydrous sodium sulfate, filtered and concentrated under reduced pressure to give the crude product as brown liquid.

The crude product was pre-absorbed on silica, loaded on the pre-packed orochem 40 g column and eluted at 25-30% of ethyl acetate in PET ether for 30 minutes with flow rate 20 ml/min. The appropriate fractions were collected and concentrated under reduced pressure to afford methyl 2′-chloro-6-ethyl-5′-methoxy-[3,4′-bipyridine]-4-carboxylate (500 mg, 1.508 mmol, 56.8% yield) as brown solid.

MS (ESI) calculated for (C₁₅H₁₅ClN₂O₃) (M+1)⁺, 307.09. found, 307.2.

### Step-4: Synthesis of 2′-chloro-6-ethyl-5′-methoxy-[3,4′-bipyridine]-4-carboxylic Acid

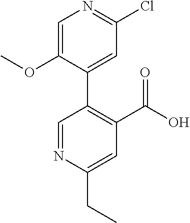 [SMILES: CCc1cc(C(=O)O)c(-c2cc(Cl)ncc2OC)cn1]

To a stirred solution of methyl 2′-chloro-6-ethyl-5′-methoxy-[3,4′-bipyridine]-4-carboxylate (500 mg, 1.630 mmol) in tetrahydrofuran (10 mL) and water (2.000 mL) was added lithiumhydroxide monohydrate (342 mg, 8.15 mmol) at room temperature. The reaction was stirred at room temperature for 5 hours. After completion, the reaction mixture was concentrated under reduced pressure. The resulting residue was diluted with water (10 mL) and washed with ethyl acetate (40 mL). The aqueous part was acidified by 1.5N HCl (˜pH 2) and extracted with 20% MeOH in DCM (60 mL). The organic phases were combined and dried over anhydrous sodium sulfate, filtered and concentrated under reduced pressure to afford 2′-chloro-6-ethyl-5′-methoxy-[3,4′-bipyridine]-4-carboxylic acid (400 mg, 1.306 mmol, 80% yield; LCMS purity 80%) as white solid.

MS (ESI) calculated for (C₁₄H₁₃ClN₂O₃) (M+1)⁺, 293.07. found, 293.0.

### Step-5: Synthesis of 2′-chloro-6-ethyl-5′-methoxy-N-(5-methoxy-1,3,4-thiadiazol-2-yl)-[3,4′-bipyridine]-4-carboxamide

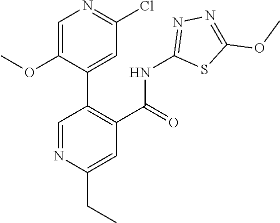

To a stirred solution of 2′-chloro-6-ethyl-5′-methoxy-[3,4′-bipyridine]-4-carboxylic acid (70 mg, 0.239 mmol) in acetonitrile (5 mL) and N, N-Dimethylformamide (0.5 mL) was added Chloro-N,N,N′,N′-tetramethylformamidinium hexafluorophosphate (81 mg, 0.287 mmol), 1-methyl-1H-imidazole (0.057 mL, 0.717 mmol) and 5-methoxy-1,3,4-thiadiazol-2-amine (31.4 mg, 0.239 mmol) at room temperature. The reaction was stirred at room temperature for 3 hours. After completion of the reaction, the reaction mixture was concentrated under vacuum. The residue was diluted with water (10 mL) and extracted with ethyl acetate (2×20 mL). The combined organic layers were washed with water (5×10 mL) and brine. The organic layer was dried over anhydrous sodium sulfate, filtered and concentrated under reduced pressure to give the crude product as yellow viscous liquid.

The crude product was purified by GRACE revelleris X2 (reverse phase) with the following condition: (Column: 40 g Grace C18 catriage; Mobile Phase A: 0.1% NH4HCO3 in water, Mobile Phase B: acetonitrile) 0-100% of B in A over 0-50 min at Flow rate: 15 mL/min. The pure product was eluted at 40-45% of B in A. The appropriate fractions were combined and evaporated in vacuum to afford 2′-chloro-6-ethyl-5′-methoxy-N-(5-methoxy-1,3,4-thiadiazol-2-yl)-[3,4′-bipyridine]-4-carboxamide (60 mg, 0.147 mmol, 61.4% yield) as white solid.

MS (ESI) calculated for (C₁₇H₁₆ClN₅O₃S) (MH)⁺, 406.07. found, 406.0.

1H-NMR (400 MHz, DMSO-d6): δ 13.00 (s, 1H), 8.58 (s, 1H), 8.14 (s, 1H), 7.66 (s, 1H), 7.56 (s, 1H), 4.08 (s, 3H), 3.61 (s, 3H), 2.89 (q, J=7.60 Hz, 2H), 1.31 (t, J=7.60 Hz, 3H).

### Example 276

### 2′-chloro-6-ethoxy-5′-methoxy-N-(5-methoxy-1,3,4-thiadiazol-2-yl)-[3,4′-bipyridine]-4-carboxamide

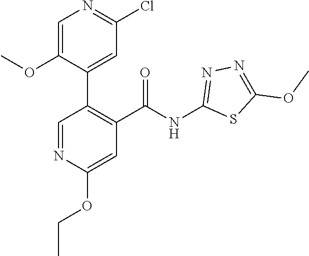

### Step-1: Synthesis of methyl 5-bromo-2-ethoxyisonicotinate

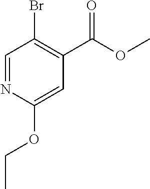

To a stirred solution of methyl 5-bromo-2-hydroxyisonicotinate (700 mg, 3.02 mmol) in Toluene (15 mL) under nitrogen was added silver carbonate (1081 mg, 3.92 mmol) followed by iodoethane (0.364 mL, 4.53 mmol). The reaction mixture was stirred at 100° C. for 1 hour. After completion of the reaction, reaction mixture was cooled to room temperature and passed through celite bed and the celite bed was washed with ethyl acetate (125 mL). The filtrate was washed with water (100 mL) and brine (50 mL). The organic layer was dried over Na₂SO₄, filtered and concentrated under reduced pressure to afford crude product as yellow liquid.

The pre-absorbed crude product was loaded on the pre-packed column (orochem, 12 g) and eluted at 0-30% of ethyl acetate in Pet ether for 20 minutes with flow rate 25 ml/min. The pure product was eluated at 5% ethyl acetate in Pet ether. The appropriate fractions were collected and concentrated under reduced pressure to afford methyl 5-bromo-2-ethoxyisonicotinate (700 mg, 2.61 mmol, 87% yield) as a white solid

MS (ESI) calculated for (C₉H₁₀BrNO₃) (M+1)⁺, 259.99. found, 260.0.

1H-NMR (400 MHz, DMSO-d⁶): δ 8.46 (s, 1H), 7.14 (s, 1H), 4.32 (q, J=7.2 Hz, 2H), 3.89 (s, 3H), 1.32 (t, J=7.2 Hz, 3H)

### Step-2: Synthesis of methyl 2′-chloro-6-ethoxy-5′-methoxy-[3,4′-bipyridine]-4-carboxylate

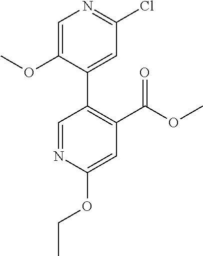

To a stirred solution of methyl 5-bromo-2-ethoxyisonicotinate (700 mg, 2.69 mmol) in 1,4-dioxane (15 mL) and water (2 mL) were added (2-chloro-5-methoxypyridin-4-yl)boronic acid (756 mg, 4.04 mmol) and potassium carbonate (744 mg, 5.38 mmol) at room temperature and the reaction mixture was degassed with nitrogen for 10 minutes. PdCl2(dppf) (98 mg, 0.135 mmol) was added and the reaction mixture was stirred at 100° C. for 2 hours. After completion of the reaction, the reaction mixture was cooled to room temperature, inorganic solids were filtered through celite pad and washed with ethyl acetate (100 mL). The filtrate was washed with water (80 mL), dried over sodium sulfate, filtered and concentrated under reduced pressure to afford crude product as brown color liquid.

The pre-absorbed crude product was loaded on the Biotage 24 g snap and eluted at 20% of ethyl acetate in pet ether for 10 minutes with flow rate 20 ml/min. The appropriate fractions were collected and concentrated under reduced pressure to afford methyl 2′-chloro-6-ethoxy-5′-methoxy-[3,4′-bipyridine]-4-carboxylate (570 mg, 1.698 mmol, 63.1% yield) as a white solid

MS (ESI) calculated for (C₁₅H₁₅ClN₂O₄) (M+1)⁺, 323.08. found, 323.0.

### Step-3: Synthesis of 2′-chloro-6-ethoxy-5′-methoxy-[3,4′-bipyridine]-4-carboxylic Acid

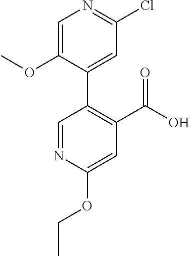 [SMILES: CCOc1cc(C(=O)O)c(-c2cc(Cl)ncc2OC)cn1]

To a stirred solution of methyl 2′-chloro-6-ethoxy-5′-methoxy-[3,4′-bipyridine]-4-carboxylate (670 mg, 2.076 mmol) in mixture of tetrahydrofuran (5.0 mL) and methanol (5.0 mL) was added lithium hydroxide (149 mg, 6.23 mmol) in water (1 mL) dropwise at 0° C. The reaction mixture was stirred at room temperature for 16 hours. After completion, the reaction mixture was concentrated under vacuum. The resulting residue was diluted with water (50 mL) and washed with DCM (100 mL) to remove impurities, the aqueous layer was acidified with saturated aqueous solution of citric acid upto pH 5-6 and extracted with ethyl acetate (2×50 mL). The combined organic layer was dried over anhydrous sodium sulfate, filtered and concentrated under reduced pressure to afford 2′-chloro-6-ethoxy-5′-methoxy-[3,4′-bipyridine]-4-carboxylic acid (570 mg, 1.837 mmol, 88% yield) as white solid.

MS (ESI) calculated for (C₁₄H₁₃ClN₂O₄) (M+1)⁺, 309.06. found, 309.0.

### Step-4: Synthesis of 2′-chloro-6-ethoxy-5′-methoxy-N-(5-methoxy-1,3,4-thiadiazol-2-yl)-[3,4′-bipyridine]-4-carboxamide

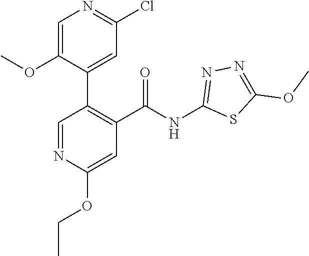

To a stirred solution of 2′-chloro-6-ethoxy-5′-methoxy-[3,4′-bipyridine]-4-carboxylic acid (100 mg, 0.324 mmol), in acetonitrile (4 mL) and N,N-Dimethylformamide (0.4 mL) were added 1-methyl-1H-imidazole (106 mg, 1.296 mmol), Chloro-N,N,N′,N′-tetramethylformamidinium hexafluorophosphate (136 mg, 0.486 mmol), and 5-methoxy-1,3,4-thiadiazol-2-amine (42.5 mg, 0.324 mmol) at room temperature. The reaction mixture was stirred at same temperature for 16 h. The reaction mixture was diluted with water, extracted with ethyl acetate (2×25 mL). The combined organic layer was washed with brine, dried over anhydrous sodium sulfate, filtered and concentrated under vacuum to afford crude product as yellow liquid.

The pre-absorbed crude product was loaded on the Orochem 12 g pre-packed column and eluted at 45% of ethyl acetate in pet ether for 30 minutes with flow rate 25 ml/min. The appropriate fractions were collected and concentrated under reduced pressure to afford product as a white solid.

The solid was dissolved in 10% methanol in dichloromethane (20 mL) and washed with brine solution (25 mL), the organic layer was dried using sodium sulfate and concentrated under reduced pressure to afford 2′-chloro-6-ethoxy-5′-methoxy-N-(5-methoxy-1,3,4-thiadiazol-2-yl)-[3,4′-bipyridine]-4-carboxamide (70 mg, 0.162 mmol, 50% yield).

MS (ESI) calculated for (C₁₇H₁₆ClN₅O₄S) (M+1)⁺, 422.07. found, 422.0.

1H-NMR (400 MHz, DMSO-d⁶): δ 12.96 (s, 1H), 8.28 (s, 1H), 8.10 (s, 1H), 7.54 (s, 1H), 7.16 (s, 1H), 4.42 (q, J=7.20 Hz, 2H), 4.08 (s, 3H), 3.59 (s, 3H), 1.37 (t, J=7.20 Hz, 3H).

### Example 277 and 298

### (S)-4-(5-chloro-2-methoxy-4-(methylsulfinyl)phenyl)-N-(5-methoxy-1,3,4-thiadiazol-2-yl)-6-methylnicotinamide and (R)-4-(5-chloro-2-methoxy-4-(methylsulfinyl)phenyl)-N-(5-methoxy-1,3,4-thiadiazol-2-yl)-6-methylnicotinamide

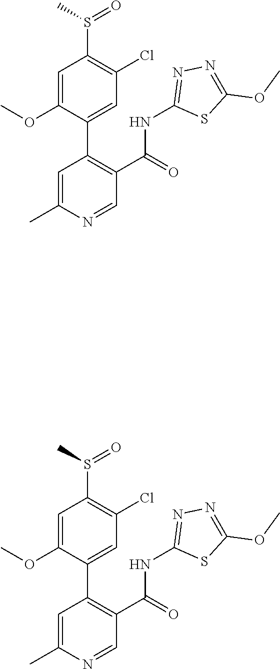

### Boronate Ester Preparation:

### Step-1: Synthesis of methyl 4-(5,5-dimethyl-1,3,2-dioxaborinan-2-yl)-6-methylnicotinate

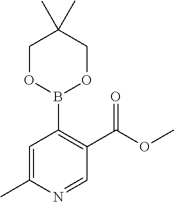

To a stirred solution of methyl 4-chloro-6-methylnicotinate (10 g, 53.9 mmol) in 1,4-Dioxane (100 mL) were added 5,5,5′,5′-tetramethyl-2,2′-bi(1,3,2-dioxaborinane) (18.26 g, 81 mmol), tert-butyldiphenylphosphane (2.61 g, 10.78 mmol) and potassium acetate (15.86 g, 162 mmol) at room temperature and degassed with nitrogen for 20 minutes. PdOAc2 (1.210 g, 5.39 mmol) was added and the mixture was again degassed with nitrogen for 5 minutes. The reaction mixture was stirred at 100° C. for 3 hours. The reaction mixture was cooled to room temperature, inorganic solids were filtered through celite pad and washed with ethyl acetate (500 mL). The filtrate was concentrated under vacuum to afford crude product as brown oil.

The crude product was pre-absorbed on silica (using 20 mL DCM, 50 g of silica (60-120 mesh), loaded on the pre-packed Orochem 120 g column and eluted at 25-40% of ethyl acetate in PET ether for 45 minutes with flow rate 40 ml/min. The appropriate fractions were collected and concentrated under reduced pressure to afford methyl 4-(5,5-dimethyl-1,3,2-dioxaborinan-2-yl)-6-methylnicotinate (14.1 g, 47.7 mmol, 89% yield) as brown gum.

MS (ESI) calculated for (C₁₃H₁₈BNO₄) (M+1)⁺, 264.14. found, 264.1.

### Acid Preparation:

### Step-2: Synthesis of (4-bromo-2-chloro-5-methoxyphenyl)(methyl)sulfane

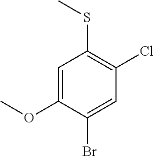

A solution of 4-bromo-2-chloro-5-methoxyaniline (2.5 g, 10.57 mmol) in 2N HCl (26.4 mL, 52.9 mmol), was heated to 50° C. for 30 min, then the reaction mixture was cooled to 0° C., and added sodium nitrite (0.802 g, 11.63 mmol) in water (20 mL) dropwise. The resulting reaction mixture was stirred for 40 min at 0° C., then sodium thiomethoxide (1.482 g, 21.14 mmol) in water (20 mL) was added dropwise at 0° C. The reaction mixture was stirred again for 18 h at room temperature. After completion of the reaction, The reaction mixtures was diluted with 10% NaOH aq. solution (50 mL) and extracted with EtOAc (2×200 mL). The separated organic layer was dried over anhydrous Na2SO4 and concentrated under vacuum to afford crude product as light yellow oil.

The crude product was pre-absorbed on silica (using 10 mL DCM, 10 g of silica (60-120 mesh), loaded on the pre-packed Orochem 120 g column and eluted at 0-30% of ethyl acetate in PET ether for 45 minutes with flow rate 20 ml/min. The appropriate fractions were collected and concentrated under reduced pressure to afford (4-bromo-2-chloro-5-methoxyphenyl)(methyl)sulfane (2.1 g, 6.99 mmol, 66.1% yield) as yellow solid.

LCMS calculated for (C₈H₈BrClOS) (M)⁺, 267.91. found, (m/z) not ionized.

1H-NMR (400 MHz, DMSO-d⁶): δ 7.67 (s, 1H), 6.88 (s, 1H), 3.93 (s, 3H), 2.57 (s, 3H).

### Step-3: Synthesis of 1-bromo-5-chloro-2-methoxy-4-(methylsulfinyl)benzene

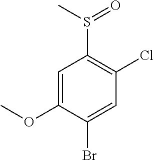

To a stirred solution of (4-bromo-2-chloro-5-methoxyphenyl)(methyl)sulfane (2.1 g, 7.85 mmol) in Dichloromethane (30 mL) was added mCPBA (1.490 g, 8.63 mmol) portion wise at 0° C., under nitrogen atmosphere. The resulting mixture was stirred at room temperature for 16 h. The reaction mixtures was diluted with 10% Na2CO3 aq. solution (100 mL) and extracted with EtOAc (2×300 mL), washed with brine. The organic layer was separated and dried over anhydrous Na₂SO₄, concentrated under vacuum to afford crude product as light yellow oil. The crude product was pre-absorbed on silica (using 15 mL DCM, 9 g of silica (60-120 mesh), loaded on the pre-packed Orochem 50 g column and eluted at 0-100% of ethyl acetate in PET ether for 60 minutes with flow rate 15 ml/min. The appropriate fractions were collected and concentrated under reduced pressure to afford 1-bromo-5-chloro-2-methoxy-4-(methylsulfinyl)benzene (1.5 g, 5.27 mmol, 67.2% yield) as yellow solid.

MS (ESI) calculated for (C₈H₈BrClO₂S) (M+1)⁺, 282.92. found, 283.0.

### Step-4: Synthesis of methyl 4-(5-chloro-2-methoxy-4-(methylsulfinyl)phenyl)-6-methylnicotinate

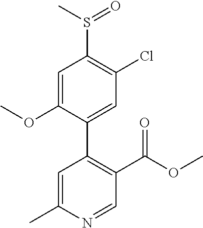

To a stirred solution of 1-bromo-5-chloro-2-methoxy-4-(methylsulfinyl)benzene (500 mg, 1.763 mmol) in Toluene (30 mL) were added methyl 4-(5,5-dimethyl-1,3,2-dioxaborinan-2-yl)-6-methylnicotinate (1392 mg, 5.29 mmol) and potassium carbonate (731 mg, 5.29 mmol) at room temperature and degassed with nitrogen for 10 minutes. To the resulting reaction mixture was added PdCl2(dppf) (258 mg, 0.353 mmol). The reaction mixture was stirred at 120° C. for 16 hours.

After completion of the reaction, the reaction mixture was cooled to room temperature, The solvent was evaporated then the reaction mixture was dissolved in water (100 mL) and extracted with EtOAc (100×2 mL), washed with brine water (30 mL), dried over sodium sulfate, filtered and concentrated under reduced pressure to afford crude product as brown semi solid.

The crude product was mixed with another batch of (500 mg) and combined 2 batched pre-absorbed on silica (using 20 mL DCM, 10 g of silica (60-120 mesh), loaded on the pre-pack orochem 50 g column and eluted at 2-10% of methanol in DCM for 60 minutes with flow rate 25 ml/min. The appropriate fractions were collected and concentrated under reduced pressure to afford methyl 4-(5-chloro-2-methoxy-4-(methylsulfinyl)phenyl)-6-methylnicotinate (1.3 g, 2.83 mmol) as a brown solid.

MS (ESI) calculated for (C₁₆H₁₆ClNO₄S) (M+1)⁺, 354.06. found, 354.0.

### Step-5: Synthesis of rac-4-(5-chloro-2-methoxy-4-(methylsulfinyl)phenyl)-6-methylnicotinic Acid

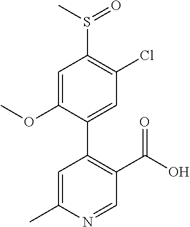 [SMILES: COc1cc(S(C)=O)c(Cl)cc1-c1cc(C)ncc1C(=O)O]

To a stirred solution of methyl methyl 4-(5-chloro-2-methoxy-4-(methylsulfinyl)phenyl)-6-methylnicotinate (900 mg, 2.54 mmol) in a mixture of Tetrahydrofuran (1.5 mL), Methanol (1 mL) and Water (0.5 mL) was added lithiumhydroxide (747 mg, 17.81 mmol) at room temperature. The reaction mixture was stirred at room temperature for 2 hours. After completion of the reaction, the reaction mixture was concentrated under vacuum. The resulting residue diluted with water (50 mL) and washed with EtOAc (25 mL). The aqueous layer was acidified with 1.5 N HCl upto pH-6 and extracted with 10% MeOH in DCM (3×150 mL). The combined organic layer dried over anhydrous sodium sulfate, filtered and concentrated under reduced pressure to afford rac-4-(5-chloro-2-methoxy-4-(methylsulfinyl)phenyl)-6-methylnicotinic acid (750 mg, 2.185 mmol, 86% yield) as grey solid.

MS (ESI) calculated for (C₁₅H₁₄ClNO₄S) (M+1)⁺, 340.04. found, 340.0.

### Step-6: Separation of (R)-4-(5-chloro-2-methoxy-4-(methylsulfinyl)phenyl)-6-methylnicotinic acid and (S)-4-(5-chloro-2-methoxy-4-(methylsulfinyl)phenyl)-6-methylnicotinic Acid

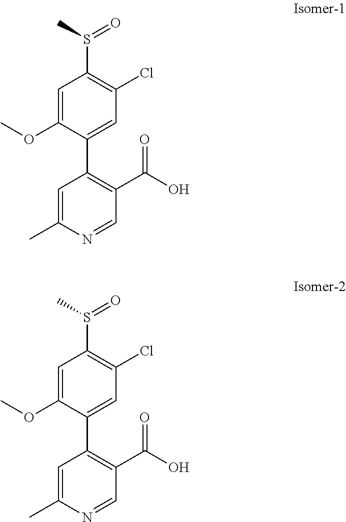 [SMILES: COc1cc([S@@](C)=O)c(Cl)cc1-c1cc(C)ncc1C(=O)O ; COc1cc([S@](C)=O)c(Cl)cc1-c1cc(C)ncc1C(=O)O]

Rac-4-(5-chloro-2-methoxy-4-(methylsulfinyl)phenyl)-6-methylnicotinic acid (650 mg) was purified by Chiral SFC by using the following method:

Column: Chiralpak IG (250*30) mm, 5 μm, Mobile Phase: CO2: MeOH (70:30)%, Total Flow: 100 g/min, Back pressure: 100 bar, Wave length: 220 nm, Cycle time: 5.5 min, 650 mg of sample was dissolved in 8.0 ml of MeOH/THF and injected 700 μl/injection;

After SFC purification, the two appropriate fractions were collected.

Fraction-1 was concentrated under reduced pressure to afford (R)-4-(5-chloro-2-methoxy-4-(methylsulfinyl)phenyl)-6-methylnicotinic acid-Isomer-1 (270 mg, 0.763 mmol, 39.9% yield) as an off-white solid. Chiral SFC purity: 100%; Rt=2.4 mins;

MS (ESI) calculated for (C₁₅H₁₄ClNO₄S) (M+1)⁺, 340.04. found, 340.0.

Fraction-2 was concentrated under reduced pressure to afford (S)-4-(5-chloro-2-methoxy-4-(methylsulfinyl)phenyl)-6-methylnicotinic acid (290 mg, 0.819 mmol, 42.8% yield) as an off-white solid. Chiral SFC purity: 99.74%; Rt=3.29 mins;

MS (ESI) calculated for (C₁₅H₁₄ClNO₄S) (M+1)⁺, 340.04. found, 340.0.

The absolute stereochemistry was not determined.

### Acid-Amine Coupling:

### Step-6: Synthesis of (S)-4-(5-chloro-2-methoxy-4-(methylsulfinyl)phenyl)-N-(5-methoxy-1,3,4-thiadiazol-2-yl)-6-methylnicotinamide

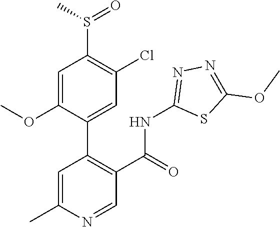

To a stirred solution of (S)-4-(5-chloro-2-methoxy-4-(methylsulfinyl)phenyl)-6-methylnicotinic acid-Isomer-2 (80 mg, 0.235 mmol), in Acetonitrile (3 mL) and N,N-Dimethylformamide (0.5 mL) were added 1-methyl-1H-imidazole (77 mg, 0.942 mmol), Chloro-N,N,N′,N′-tetramethylformamidiniumhexafluorophosphate (99 mg, 0.353 mmol), and 5-methoxy-1,3,4-thiadiazol-2-amine (37.1 mg, 0.283 mmol) at room temperature. The reaction mixture was stirred at room temperature for 16 h. After completion the reaction mixture was quenched with cold water and evaporated all the volatiles. Then diluted with ethyl acetate and extracted by ethyl acetate (150 mL×3). The combined organic layer dried over anhydrous sodium sulfate, filtered and concentrated under vacuum to afford crude product as brown solid.

The crude product was purified by prep-HPLC with the following condition: (Atlantis C18 (19*250 mm) 5 MICRON; Mobile Phase A: 10 mM ABC in Milli Q Water 70%, Mobile Phase B: Acetonitrile 30%); Flow: 12.00 ml/min; Injection Volume: 200.00 μL; Number of injection: 14; Diluent: THF/ACN; Run time: 19 min; to afford (S)-4-(5-chloro-2-methoxy-4-(methylsulfinyl)phenyl)-N-(5-methoxy-1,3,4-thiadiazol-2-yl)-6-methylnicotinamide (40 mg, 0.087 mmol, 37.1% yield) as a off-white solid. The absolute stereochemistry was not determined.

MS (ESI) calculated for (C₁₈H₁₇ClN₄O₄S₂) (M+1)⁺, 453.05. found, 453.0.

1H-NMR (400 MHz, DMSO-d⁶): δ 12.85 (s, 1H), 8.75 (s, 1H), 7.61 (s, 1H), 7.40 (s, 1H), 7.34 (s, 1H), 4.07 (s, 3H), 3.61 (s, 3H), 2.86 (s, 3H), 2.58 (s, 3H).

### Step-6: Synthesis of (R)-4-(5-chloro-2-methoxy-4-(methylsulfinyl)phenyl)-N-(5-methoxy-1,3,4-thiadiazol-2-yl)-6-methylnicotinamide

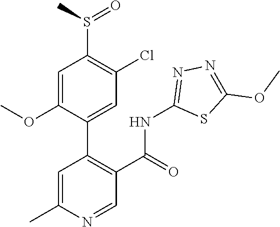

To a stirred solution of (R)-4-(5-chloro-2-methoxy-4-(methylsulfinyl)phenyl)-6-methylnicotinic acid (80 mg, 0.235 mmol), in Acetonitrile (5 mL) and N,N-Dimethylformamide (1 mL) were added 1-methyl-1H-imidazole (77 mg, 0.942 mmol), Chloro-N,N,N′,N′-tetramethylformamidiniumhexafluorophosphate (99 mg, 0.353 mmol), and 5-methoxy-1,3,4-thiadiazol-2-amine (37.1 mg, 0.283 mmol) at room temperature. The reaction mixture was stirred at room temperature for 4 h. After completion the reaction mixture was quenched with cold water and evaporated all the volatiles. Then diluted with ethyl acetate and extracted by ethyl acetate (50 mL×3). The combined organic layer dried over anhydrous sodium sulfate, filtered and concentrated under vacuum to afford crude product as orange solid.

The crude product was purified by prep-HPLC with the following condition: (X-select (19*250 mm) 5 MICRON; Mobile Phase A: 0.1% FA in MQ water 80%, Mobile Phase B: Acetonitrile 20%); Flow: 12.00 ml/min; Injection Volume: 200 μL; Number of injection: 16; Diluent: THF/ACN; Run time: 18 min; pure product was lyophilized to afford (R)-4-(5-chloro-2-methoxy-4-(methylsulfinyl)phenyl)-N-(5-methoxy-1,3,4-thiadiazol-2-yl)-6-methylnicotinamide (32 mg, 0.07 mmol, 29.6% yield) as light yellow solid. The absolute stereochemistry was not determined.

MS (ESI) calculated for (C₁₈H₁₇ClN₄O₄S₂) (M+1)⁺, 453.05. found, 453.0.

1H-NMR (400 MHz, DMSO-d⁶): δ 12.83 (s, 1H), 8.75 (s, 1H), 7.62 (s, 1H), 7.40 (s, 1H), 7.34 (s, 1H), 4.08 (s, 3H), 3.61 (s, 3H), 2.86 (s, 3H), 2.59 (s, 3H).

### Example 294

### 4-(2-chloro-5-methoxypyridin-4-yl)-N-(5-methoxy-1,3,4-thiadiazol-2-yl)-6-methylpyridazine-3-carboxamide

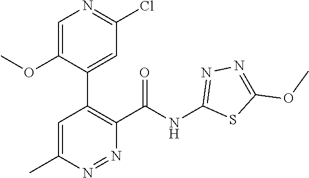

### Step-1: Synthesis of methyl 2-diazo-3-oxobutanoate

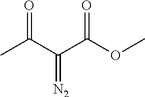

To a stirred solution of methyl 3-oxobutanoate (5 g, 43.1 mmol), in acetonitrile (50 mL) were added successively triethylamine (7.80 mL, 56.0 mmol) and 4-acetamidobenzenesulfonyl azide (10.34 g, 43.1 mmol) at 0° C. The reaction mixture was stirred at 0° C. for 2 hours. After completion of the reaction, the reaction mixture was allowed to room temperature, diluted with MTBE/hexane (1:1) 100 mL and then filtered. The filterate was concentrated under reduced pressure to afford crude residue as yellow gum.

The crude product was pre-absorbed on silica, loaded on the pre-packed Biotage column (50 g) and eluted at 10-20% of ethyl acetate in Pet ether for 60 minutes with flow rate 30 ml/min. The appropriate fractions were collected and concentrated under reduced pressure to afford methyl 2-diazo-3-oxobutanoate (5.1 g, 35.9 mmol, 83% yield) as yellow oil.

MS (ESI) calculated for (C₅H₆N₂O₃) (M+1)⁺, 143.05. found, poor ionization.

1H-NMR (400 MHz, DMSO-d⁶): δ 3.78 (s, 3H), 2.38 (s, 3H)

### Step-2: Synthesis of methyl 2-diazo-5-hydroxy-3-oxohexanoate

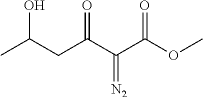

To a stirred solution of methyl 2-diazo-3-oxobutanoate (6 g, 42.2 mmol), in dichloromethane (60 mL) were added successively Titanium tetrachloride (TiCl4) (5.12 mL, 46.4 mmol) and triethylamine (6.47 mL, 46.4 mmol) at −78° C. The reaction mixture was stirred at −78° C. for 1 hour. After 1 hour a solution of acetaldehyde (2.384 mL, 42.2 mmol) in dichloromethane (5 mL) was added at −78° C. and stirred at same temperature for 2 hours. After completion, the reaction mixture was quenched with saturated NH₄Cl solution (50 mL) and extracted with DCM (2×100 mL). The combined organic layers were, dried over sodium sulfate, filtered and concentrated under reduced pressure to afford crude product as yellow liquid.

The crude product was pre-absorbed on silica, loaded on the pre-packed Biotage column (100 g) and eluted at 20-40% of ethyl acetate in pet ether for 60 minutes with flow rate 40 ml/min. The appropriate fractions were collected and concentrated under reduced pressure to afford methyl 2-diazo-5-hydroxy-3-oxohexanoate (6.1 g, 32.5 mmol, 77% yield) as yellow oil.

MS (ESI) calculated for (C₇H₁₀N₂O₄) (M+1)⁺, 187.07. found, 187.0.

### Step-3: Synthesis of methyl 2-diazo-3,5-dioxohexanoate

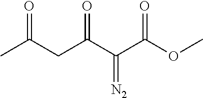

To a stirred solution of methyl 2-diazo-5-hydroxy-3-oxohexanoate (5 g, 26.9 mmol), in acetonitrile (50 mL) was added 2-Iodoxybenzoic acid (IBX) (20.05 g, 32.2 mmol) at room temperature. The reaction mixture was stirred at 80° C. for 2 hours. After completion of the reaction, the reaction mixture was allowed to room temperature, and then filtered. The filterate was concentrated under reduced pressure to afford crude residue as yellow gum.

The crude product was pre-absorbed on silica, loaded on the pre-packed Biotage column (50 g) and eluted at 10-20% of ethyl acetate in pet ether for 60 minutes with flow rate 35 ml/min. The appropriate fractions were collected and concentrated under reduced pressure to afford methyl 2-diazo-3,5-dioxohexanoate (5.1 g, 14.49 mmol, 53.9% yield; LCMS purity: 52.31%) as an off white solid

MS (ESI) calculated for (C₇H₃N₂O₄) (M+1)⁺, 185.06. found, 185.0.

### Step-4: Synthesis of methyl 4-hydroxy-6-methylpyridazine-3-carboxylate

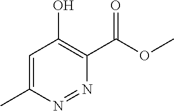

To a stirred solution of methyl 2-diazo-3,5-dioxohexanoate (5.10 g, 14.49 mmol), in t-butylmethyl ether (50 mL) was added tri-n-butylphosphine (3.57 mL, 14.49 mmol) at room temperature. The reaction mixture was stirred at room temperature for 30 minutes. After completion of the reaction, the resulting precipitate was collected and dried under reduced pressure to afford product methyl 4-hydroxy-6-methylpyridazine-3-carboxylate (2.2 g, 12.64 mmol, 87% yield) as an off white solid.

MS (ESI) calculated for (C₇H₈N₂O₃) (M+1)⁺, 169.06. found, 169.0.

### Step-5: Synthesis of methyl 4-chloro-6-methylpyridazine-3-carboxylate

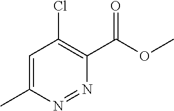

To a solution of methyl 4-hydroxy-6-methylpyridazine-3-carboxylate (2.2 g, 13.08 mmol), in POCl3 (20 mL, 215 mmol) was stirred at 100° C. for 1 hour. After completion of the reaction,

The reaction mixture was cooled to room temperature and excess of POCl3 was distilled out under reduced pressure to afford crude residue. The residue was quenched with saturated sodium bicarbonate solution (50 mL) and extracted with ethyl acetate (2×100 mL). The combined organic layers were, dried over sodium sulfate, filtered and concentrated under reduced pressure to afford crude product as yellow gum.

The crude product was pre-absorbed on silica, loaded on the Orochem 40 g snap and eluted at 20-50% of ethyl acetate in pet ether for 60 minutes with flow rate 30 ml/min. The appropriate fractions were collected and concentrated under reduced pressure to afford product methyl 4-chloro-6-methylpyridazine-3-carboxylate (1.5 g, 7.99 mmol, 61.1% yield) as pale yellow solid.

MS (ESI) calculated for (C₇H₇ClN₂O₂) (M+1)⁺, 187.03. found, 187.0.

### Step-6: Synthesis of methyl 4-(2-chloro-5-methoxypyridin-4-yl)-6-methylpyridazine-3-carboxylate

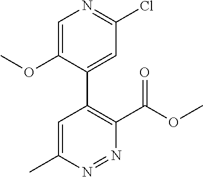

To a stirred solution of methyl 4-chloro-6-methylpyridazine-3-carboxylate (1.5 g, 8.04 mmol) in 1,4-Dioxane (20 mL) were added (2-chloro-5-methoxypyridin-4-yl)boronic acid (2.260 g, 12.06 mmol) and potassium carbonate (3.33 g, 24.12 mmol) at room temperature and the reaction mixture was degassed with nitrogen for 5 minutes. After degassing, PdCl2(dppf) (0.588 g, 0.804 mmol) was added and the reaction mixture was again degassed with nitrogen for 5 minutes. Then the reaction mixture was stirred at 80° C. for 16 hours. After completion, the reaction mixture was cooled to room temperature, inorganic solids were filtered through celite pad and washed with ethyl acetate (100 mL). The filtrate was washed with water (50 mL), dried over sodium sulfate, filtered and concentrated under reduced pressure to afford crude product as brown gum.

The crude product was pre-absorbed on silica, loaded on the pre-packed Orochem column (25 g) and eluted at 50-60% of ethyl acetate in pet ether for 60 minutes with flow rate 30 ml/min. The appropriate fractions were collected and concentrated under reduced pressure to afford methyl 4-(2-chloro-5-methoxypyridin-4-yl)-6-methylpyridazine-3-carboxylate (1.3 g, 4.37 mmol, 54.3% yield) as pale yellow solid.

MS (ESI) calculated for (C₁₃H₁₂ClN₃O3) (M+1)⁺, 294.06. found, 294.0.

### Step-7: Synthesis of 4-(2-chloro-5-methoxypyridin-4-yl)-6-methylpyridazine-3-carboxylic Acid

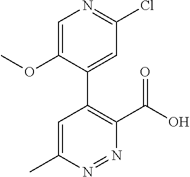 [SMILES: COc1cnc(Cl)cc1-c1cc(C)nnc1C(=O)O]

To a stirred solution of methyl 4-(2-chloro-5-methoxypyridin-4-yl)-6-methylpyridazine-3-carboxylate (1.3 g, 4.43 mmol) in tetrahydrofuran (15 mL) was added lithium hydroxide monohydrate (0.929 g, 22.13 mmol) and water (5 mL). Then the reaction mixture was stirred at room temperature for 2 hours. After completion of the reaction, the reaction mixture was concentrated under reduced pressure to give crude residue. The resulting residue diluted with water (5 mL), the aqueous layer was acidified with 1.5 N HCl upto pH-6 and precipitated solids were filtered off and dried under reduced pressure to afford the product 4-(2-chloro-5-methoxypyridin-4-yl)-6-methylpyridazine-3-carboxylic acid (700 mg, 2.478 mmol, 56.0% yield) as an off white solid.

MS (ESI) calculated for (C₁₂H₁₀ClN₃O3) (M+1)⁺, 280.05. found, 280.0.

### Step-8: Synthesis of 4-(2-chloro-5-methoxypyridin-4-yl)-N-(5-methoxy-1,3,4-thiadiazol-2-yl)-6-methylpyridazine-3-carboxamide

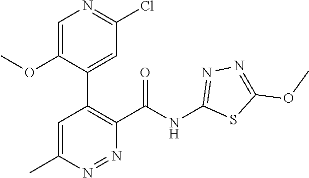

To a stirred solution of 4-(2-chloro-5-methoxypyridin-4-yl)-6-methylpyridazine-3-carboxylic acid (150 mg, 0.536 mmol), in acetonitrile (4 mL) were added Chloro-N,N,N′,N′-tetramethylformamidinium hexafluorophosphate (166 mg, 0.590 mmol), 1-methylimidazole (0.128 mL, 1.609 mmol), N,N-Dimethylformamide (DMF) (0.8 mL) and 5-methoxy-1,3,4-thiadiazol-2-amine (70.3 mg, 0.536 mmol) at room temperature. The reaction mixture was stirred at room temperature for 16 hours. After completion, the reaction mixture was diluted with water (15 mL) and extracted with 10% MeOH in DCM (2×30 mL). The combined organic layers were dried over anhydrous sodium sulfate, filtered and concentrated under reduced pressure to afford the product as yellow gum.

The crude product was pre-absorbed on silica, loaded on the pre-packed Orochem column (12 g) and eluted at 2-3% MeOH in DCM for 60 minutes with flow rate 25 ml/min. The appropriate fractions were collected and concentrated under reduced pressure to give the product. The product was dissolved in 20% ethanol in acetonitrile (3 mL), concentrated under reduced pressure to afford product to afford 4-(2-chloro-5-methoxypyridin-4-yl)-N-(5-methoxy-1,3,4-thiadiazol-2-yl)-6-methylpyridazine-3-carboxamide (90 mg, 0.227 mmol, 42.4% yield) as an off white solid.

MS (ESI) calculated for (C₁₅H₁₃ClN₆O₃S) (M+1)⁺, 393.05. found, 393.0.

1H-NMR (400 MHz, DMSO-d6): δ 13.21 (s, 1H), 8.24 (s, 1H), 7.89 (s, 1H), 7.68 (s, 1H), 4.10 (s, 3H), 3.67 (s, 3H), 2.78 (s, 3H).

### Example 311

### 2′-chloro-6-(dimethylamino)-5′-methoxy-N-(5-methoxy-1,3,4-thiadiazol-2-yl)-[3,4′-bipyridine]-4-carboxamide

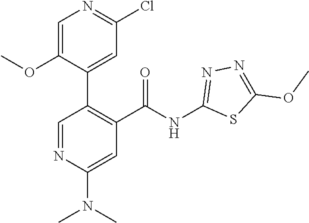

### Step-1: Synthesis of methyl 5-bromo-2-(dimethylamino)isonicotinate

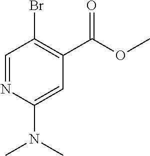

To a stirred suspension of methyl 2-amino-5-bromoisonicotinate (2.5 g, 10.82 mmol) in acetonitrile (75 mL) was added sequentially water (12.50 mL), followed by formaldehyde (24.97 mL, 335 mmol) and acetic acid (6.00 mL, 105 mmol) at 0° C. The reaction mixture was stirred for 10 mins at same temperature. And then added sodium cyanoborohydride (2.040 g, 32.5 mmol) at 0° C. The resulting reaction mixture was stirred at room temperature for 16 h. After completion, the solvent was evaporated and the residue was treated with aqueous sat. sodium bicarbonate solution (15 mL) and extracted with ethyl acetate (3×50 mL). The combined organics were washed with brine, dried over sodium sulfate, filtered and concentrated to give crude product as yellow gum.

The crude product was pre-absorbed on silica, loaded on the biotage pre-packed column (110 g) and eluted at 0-100% of ethyl acetate in Pet ether for 60 minutes with flow rate 40 ml/min. The appropriate fractions were collected and concentrated under reduced pressure to afford methyl 5-bromo-2-(dimethylamino)isonicotinate (1.02 g, 3.93 mmol, 36.3% yield) as yellow solid.

MS (ESI) calculated for (C₉H₁₁BrN₂O₂) (M+1)⁺, 259.01. found, 259.0.

### Step-2: Synthesis of methyl 2′-chloro-6-(dimethylamino)-5′-methoxy-[3,4′-bipyridine]-4-carboxylate

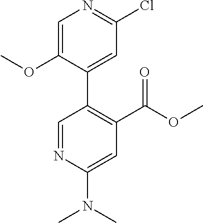

To a stirred solution of methyl methyl 5-bromo-2-(dimethylamino)isonicotinate (860 mg, 3.32 mmol) in 1,4-Dioxane (15 mL) were added (2-chloro-5-methoxypyridin-4-yl)boronic acid (933 mg, 4.98 mmol) and potassium carbonate (1376 mg, 9.96 mmol) at room temperature and degassed with nitrogen for 5 minutes. PdCl2(dppf) (486 mg, 0.664 mmol) was added and the reaction mixture was again degassed with nitrogen for 5 minutes. Then the reaction mixture was stirred at 100° C. for 16 hours. After completion of the reaction, the reaction mixture was cooled to room temperature, inorganic solids were filtered through celite pad and washed with ethyl acetate (50 mL). The filtrate was diluted with water (80 mL) and layers were separated. The water layer was extracted with ethyl acetate (2×100 mL). The combined organic layers were washed with brine (100 mL), dried over sodium sulfate, filtered and concentrated under reduced pressure to afford crude product as brown gum.

The crude product was pre-absorbed on silica, loaded on the biotage pre-packed column (50 g) and eluted at 0-100% of ethyl acetate in Pet ether for 50 minutes with flow rate 45 ml/min.

The pure product was eluated at 30% ethyl acetate in Pet ether. The appropriate fractions were collected and concentrated under reduced pressure to afford methyl 2′-chloro-6-(dimethylamino)-5′-methoxy-[3,4′-bipyridine]-4-carboxylate (590 mg, 1.787 mmol, 53.8% yield) as yellow solid.

MS (ESI) calculated for (C₁₅H₁₆ClN₃O₃) (M+1)⁺, 322.10. found, 322.0.

### Step-3: Synthesis of 2′-chloro-6-(dimethylamino)-5′-methoxy-[3,4′-bipyridine]-4-carboxylic Acid

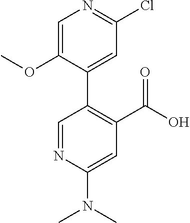 [SMILES: COc1cnc(Cl)cc1-c1cnc(N(C)C)cc1C(=O)O]

To a stirred solution of methyl 2′-chloro-6-(dimethylamino)-5′-methoxy-[3,4′-bipyridine]-4-carboxylate (590 mg, 1.834 mmol) in methanol (4 mL), and tetrahydrofuran (4 mL) was added a solution of lithiumhydroxide·monohydrate (115 mg, 2.75 mmol) in water (0.8 mL) at room temperature. The reaction mixture was stirred at room temperature for 16 hours. After completion of the reaction, the reaction mixture was diluted with water (25 mL), the aqueous was washed with EtOAc (25 mL). The aqueous layer was acidified with sat. aq. solution of citric acid and extracted with ethyl acetate (4×25 mL). The combined organic layer was washed with brine, dried over anhydrous sodium sulfate, filtered and concentrated under reduced pressure to afford 2′-chloro-6-(dimethylamino)-5′-methoxy-[3,4′-bipyridine]-4-carboxylic acid (356 mg, 1.150 mmol, 62.7% yield) as yellow solid

MS (ESI) calculated for (C₁₄H₁₄ClN₃O₃) (M+1)⁺, 308.08. found, 308.0.

### Step-4: Synthesis of 2′-chloro-6-(dimethylamino)-5′-methoxy-N-(5-methoxy-1,3,4-thiadiazol-2-yl)-[3,4′-bipyridine]-4-carboxamide

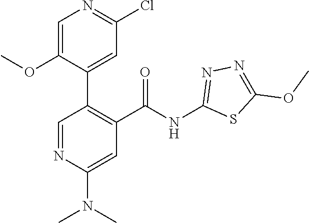

To a stirred solution of 2′-chloro-6-(dimethylamino)-5′-methoxy-[3,4′-bipyridine]-4-carboxylic acid (80 mg, 0.260 mmol), in acetonitrile (0.5 mL) and N,N-dimethylformamide (0.10 mL) were added 1-methylimidazole (0.062 mL, 0.780 mmol), Chloro-N,N,N′,N′-tetramethylformamidinium hexafluorophosphate (146 mg, 0.520 mmol), and 5-methoxy-1,3,4-thiadiazol-2-amine (34.1 mg, 0.260 mmol) at room temperature. The reaction mixture was stirred at room temperature for 3 hrs. The reaction mixture was quenched with saturated aq. sodium bicarbonate solution (10 mL). and extracted with ethyl acetate (3×15 mL). Combined the organic layer was washed with water (15 mL) and brine. The organic layer was dried over anhydrous sodium sulfate, filtered and concentrated under vacuum to afford crude product as yellow gum.

The crude product was purified by GRACE revelleris X2 (reverse phase) with the following condition: (Column: 80 g Grace C18 catriage; Mobile Phase A: 0.1% FA in water, Mobile Phase B: acetonitrile) 0-100% of B in A over 0-50 min. The pure product was eluted at 60% of B in A. The appropriate fractions were combined and evaporated in vacuum to give solid product. The resulted solid freeze was dried to afford 2′-chloro-6-(dimethylamino)-5′-methoxy-N-(5-methoxy-1,3,4-thiadiazol-2-yl)-[3,4′-bipyridine]-4-carboxamide (86 mg, 0.204 mmol, 78% yield). The structure was confirmed with X-ray crystallography.

MS (ESI) calculated for (C₁₇H₁₇ClN₆O₃S) (M+1)⁺, 421.09. found, 420.8.

1H-NMR (400 MHz, DMSO-d⁶): δ 12.85 (s, 1H), 8.16 (s, 1H), 8.03 (s, 1H), 7.45 (s, 1H), 6.97 (s, 1H), 4.08 (s, 3H), 3.57 (s, 3H), 3.15 (s, 6H).

### Example 312 and 313

### (S)-4-(5-chloro-4-(ethylsulfinyl)-2-methoxyphenyl)-N-(5-methoxy-1,3,4-thiadiazol-2-yl)-6-methylnicotinamide and (R)-4-(5-chloro-4-(ethylsulfinyl)-2-methoxyphenyl)-N-(5-methoxy-1,3,4-thiadiazol-2-yl)-6-methylnicotinamide

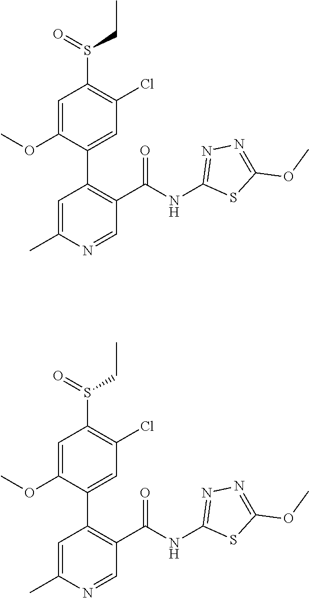

### Step-1: Synthesis of (4-bromo-2-chloro-5-methoxyphenyl)(ethyl)sulfane

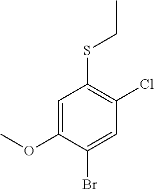

A solution of 4-bromo-2-chloro-5-methoxyaniline (2.0 g, 8.46 mmol) in 2N HCl (28.2 mL, 42.3 mmol), was heated to 50° C. for 30 min, then the reaction mixture was cooled to 0° C., and added sodium nitrite (0.642 g, 9.30 mmol) in water (20 mL) dropwise. The resulting reaction mixture was stirred for 40 min at 0° C., then sodium ethanethiolate (1.423 g, 16.91 mmol) in water (20 mL) was added dropwise at 0° C. The reaction mixture was stirred again for 18 h at room temperature. After completion of the reaction. The reaction mixtures was diluted with 10% NaOH aq. solution (50 mL) and extracted with EtOAc (2×50 mL). The separated organic layer was dried over anhydrous Na2SO4 and concentrated under vacuum to afford crude product as yellow oil.

The crude product was pre-absorbed on silica (using 10 mL DCM, 10 g of silica (60-120 mesh), loaded on the pre-packed Orochem 120 g column and eluted at 0-40% of ethyl acetate in PET ether with flow rate 20 ml/min. The appropriate fractions were collected and concentrated under reduced pressure to afford (4-bromo-2-chloro-5-methoxyphenyl)(ethyl)sulfane (1.49 g, 3.03 mmol, 35.8% yield; 57.6% purity by GCMS) as yellow solid.

LCMS calculated for (C₉H₁₀BrClOS) (M)+, 279.9. found, (m/z) not ionized.

1H-NMR (400 MHz, DMSO-d⁶): δ 7.68 (s, 1H), 6.96 (s, 1H), 3.91 (m, 3H), 3.10 (q, J=7.2 Hz, 2H), 1.30 (t, J=7.2 Hz, 3H).

### Step-2: Synthesis of 1-bromo-5-chloro-4-(ethylsulfinyl)-2-methoxybenzene

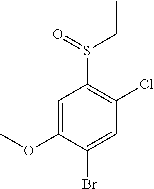

To a stirred solution of (4-bromo-2-chloro-5-methoxyphenyl)(ethyl)sulfane (3.0 g, 10.65 mmol) in Dichloromethane (30 mL) was added mCPBA (2.02 g, 11.72 mmol) portion wise at 0° C., under nitrogen atmosphere. The resulting mixture was stirred at room temperature for 14 h. The reaction mixtures was diluted with 10% sodium bicarbonate aq. solution (20 mL) and extracted with dichloromethane (2×50 mL). The organic layer was separated and dried over anhydrous Na2SO4, concentrated under vacuum to afford crude product as brown liquid.

The pre-adsorbed crude product was loaded on the pre-packed Orochem 100 g column and eluted at 0-40% of ethyl acetate in PET ether with flow rate 20 ml/min. The appropriate fractions were collected and concentrated under reduced pressure to afford 1-bromo-5-chloro-4-(ethylsulfinyl)-2-methoxybenzene (1.3 g, 4.22 mmol, 39.2% yield) as brown solid.

MS (ESI) calculated for (C₉H₁₀BrClO₂S) (M+1)⁺, 296.93. found, 296.8.

### Step-3: Synthesis of methyl 4-(5-chloro-4-(ethylsulfinyl)-2-methoxyphenyl)-6-methylnicotinate

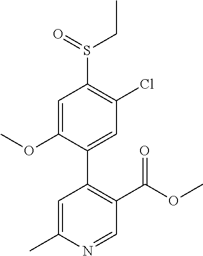

To a stirred solution 1-bromo-5-chloro-4-(ethylsulfinyl)-2-methoxybenzene (1.3 g, 4.37 mmol) in dioxane (10 mL) and water (2 mL) were added methyl 4-(5,5-dimethyl-1,3,2-dioxaborinan-2-yl)-6-methylnicotinate (2.069 g, 7.86 mmol) and potassium carbonate (1.81 g, 13.11 mmol) at room temperature, and the mixture was degassed with nitrogen for 10 minutes. PdCl2(dppf) (0.32 g, 0.437 mmol) was then added and the reaction mixture was stirred at 90° C. for 16 hours. After completion of the reaction, the reaction mixture was cooled to room temperature, and filtered through celite. The celite bed was washed with ethyl acetate (50 mL). The filtrate was diluted with water (50 mL). The layers were separated. The organic layer washed with water and brine, dried over sodium sulfate, filtered and concentrated under reduced pressure to afford crude product as brown liquid.

The pre-adsorbed crude product was loaded on the pre-pack orochem 125 g column and eluted at 5-6% of methanol in DCM with flow rate 20 ml/min. The appropriate fractions were collected and concentrated under reduced pressure to afford methyl 4-(5-chloro-4-(ethylsulfinyl)-2-methoxyphenyl)-6-methylnicotinate (1.2 g, 3.02 mmol, 69.1%) as a brown gum.

MS (ESI) calculated for (C₁₇H₁₈ClNO₄S) (M+1)⁺, 368.08. found, 367.8.

### Step-4: Synthesis of 4-(5-chloro-4-(ethylsulfinyl)-2-methoxyphenyl)-6-methylnicotinic Acid

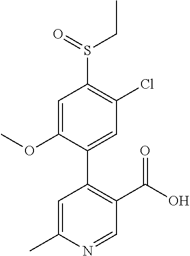 [SMILES: CCS(=O)c1cc(OC)c(-c2cc(C)ncc2C(=O)O)cc1Cl]

To a stirred solution of 4-(5-chloro-4-(ethylsulfinyl)-2-methoxyphenyl)-6-methylnicotinate (1.2 g, 3.26 mmol) in a mixture of Tetrahydrofuran (10 mL) and Water (1 mL) was added lithiumhydroxide monohydrate (0.684 g, 16.31 mmol) at room temperature. The reaction mixture was stirred at room temperature for 16 hours. After completion of the reaction, the reaction mixture was concentrated under vacuum. The resulting residue diluted with water (30 mL) and washed with EtOAc (25 mL). The aqueous layer was acidified with 1.5 N HCl upto pH-6 and extracted with 10% MeOH in DCM (2×30 mL). The combined organic layer dried over anhydrous sodium sulfate, filtered and concentrated under reduced pressure to afford 4-(5-chloro-4-(ethylsulfinyl)-2-methoxyphenyl)-6-methylnicotinic acid (1.1 g, 3.10 mmol, 95% yield) as brown solid. MS (ESI) calculated for (C₁₆H₁₆ClNO₄S) (M+1)⁺, 354.06. found, 354.0.

### Step-5: Synthesis of rac-4-(5-chloro-4-(ethylsulfinyl)-2-methoxyphenyl)-N-(5-methoxy-1,3,4-thiadiazol-2-yl)-6-methylnicotinamide

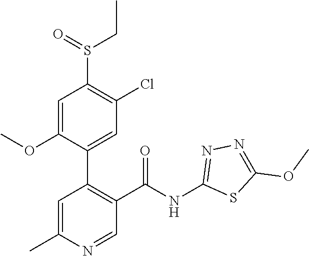

To a stirred 4-(5-chloro-4-(ethylsulfinyl)-2-methoxyphenyl)-6-methylnicotinic acid (0.6 g, 1.696 mmol) and 5-methoxy-1,3,4-thiadiazol-2-amine (0.222 g, 1.696 mmol) in Acetonitrile (10 mL) and N, N-Dimethylformamide (DMF) (2.5 mL) were added 1-methylimidazole (0.418 g, 5.09 mmol), followed by Chloro-N, N,N′,N′-tetramethylformamidinium hexafluorophosphate (0.952 g, 3.39 mmol) at room temperature under nitrogen. The reaction mixture was stirred for 16 h. The reaction mixture was quenched with cold water and extracted with 10% methanol in dichloromethane (30 mL×2). The combined organic layers were washed with water and brine, dried over anhydrous sodium sulfate, filtered and concentrated under vacuum to afford the crude product as brown liquid.

The pre-adsorbed crude product was loaded on the pre-pack orochem 25 g column and eluted at 6-8% of methanol in DCM for with flow rate 20 ml/min. The appropriate fractions were collected and concentrated under reduced pressure to afford rac-4-(5-chloro-4-(ethylsulfinyl)-2-methoxyphenyl)-N-(5-methoxy-1,3,4-thiadiazol-2-yl)-6-methylnicotinamide (0.45 g, 0.890 mmol, 52.5%) as an off-white solid.

MS (ESI) calculated for (C19H19ClN4O4S2) (M+1)⁺, 467.06. found, 467.0.

### Step-5: Separation of (S)-4-(5-chloro-4-(ethylsulfinyl)-2-methoxyphenyl)-N-(5-methoxy-1,3,4-thiadiazol-2-yl)-6-methylnicotinamide-Isomer-1 and (R)-4-(5-chloro-4-(ethylsulfinyl)-2-methoxyphenyl)-N-(5-methoxy-1,3,4-thiadiazol-2-yl)-6-methylnicotinamide-Isomer-2

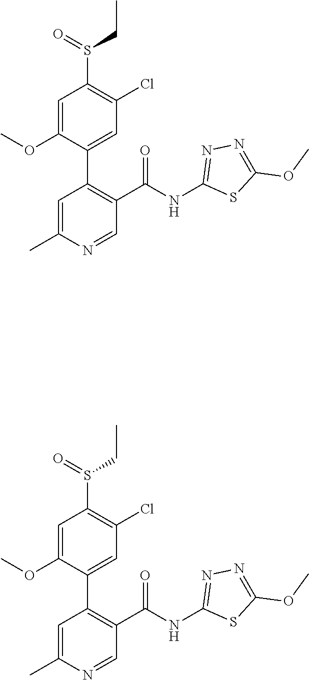

rac-4-(5-chloro-4-(ethylsulfinyl)-2-methoxyphenyl)-N-(5-methoxy-1,3,4-thiadiazol-2-yl)-6-methylnicotinamide (0.45 g) was purified by Chiral SFC by using the following method: (Column: Lux A1 (250*30) mm, 5 μm; Mobile Phase A: CO₂, Mobile Phase B: isopropyl alcohol; Gradient: isocratic 35% B; Column Temperature (° C.): 35; Back Pressure (bar): 100; Total Flow: 100 g/min; Wave Length: 220 nm; RT1 (min): 2.15; RT2 (min): 2.92; Sample Solvent: ACN/MeOH-HPLC; Injection Volume: 0.3 mL/Injection; Cycle time: 6.5 min) to afford (S)-4-(5-chloro-4-(ethylsulfinyl)-2-methoxyphenyl)-N-(5-methoxy-1,3,4-thiadiazol-2-yl)-6-methylnicotinamide (90 mg, 0.192 mmol, 19.96% yield) as white solid with the first peak on chiral SFC with shorter retention time and (R)-4-(5-chloro-4-(ethylsulfinyl)-2-methoxyphenyl)-N-(5-methoxy-1,3,4-thiadiazol-2-yl)-6-methylnicotinamide (65 mg, 0.139 mmol, 14.44% yield) as white solid with the second peak on chiral SFC with longer retention time.

The absolute stereochemistry was not determined.

### (S)-4-(5-chloro-4-(ethylsulfinyl)-2-methoxyphenyl)-N-(5-methoxy-1,3,4-thiadiazol-2-yl)-6-methylnicotinamide

MS (ESI) calculated for (C₁₉H₁₉ClN₄O₄S₂) (M+1)⁺, 467.06. found, 467.0.

1H-NMR (400 MHz, DMSO-d⁶): δ 12.82 (brs, 1H), 8.76 (s, 1H), 7.60 (s, 1H), 7.40 (s, 1H), 7.23 (s, 1H), 4.06 (s, 3H), 3.59 (s, 3H), 3.22-3.17 (m, 1H), 2.90-2.85 (m, 1H), 2.58 (s, 3H), 1.12 (t, J=7.60 Hz, 3H).

### (R)-4-(5-chloro-4-(ethylsulfinyl)-2-methoxyphenyl)-N-(5-methoxy-1,3,4-thiadiazol-2-yl)-6-methylnicotinamide

MS (ESI) calculated for (C₁₉H₁₉ClN₄O₄S₂) (M+1)⁺, 467.06. found, 467.0.

1H-NMR (400 MHz, DMSO-d⁶): δ 12.81 (s, 1H), 8.75 (s, 1H), 7.62 (s, 1H), 7.41 (s, 1H), 7.24 (s, 1H), 4.07 (s, 3H), 3.59 (s, 3H), 3.22-3.16 (m, 1H), 2.92-2.85 (m, 1H), 2.58 (s, 3H), 1.12 (t, J=7.60 Hz, 3H).

### Example 259

### 2′-chloro-5′-methoxy-N-(5-methoxy-1,3,4-thiadiazol-2-yl)-[3,4′-bipyridine]-4-carboxamide

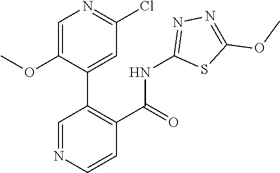

### Step-1: Synthesis of methyl 2′-chloro-5′-methoxy-[3,4′-bipyridine]-4-carboxylate

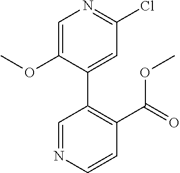 [SMILES: COC(=O)c1ccncc1-c1cc(Cl)ncc1OC]

To a stirred solution of methyl 3-bromoisonicotinate (1 g, 4.63 mmol) in 1,4-dioxane (10 mL) and water (1 mL) was added (2-chloro-5-methoxypyridin-4-yl)boronic acid (1.041 g, 5.55 mmol), potassium carbonate (0.960 g, 6.94 mmol) at room temperature. Then the reaction mixture was degassed by nitrogen gas for 10 minutes. PdCl2(dppf) (3.39 g, 4.63 mmol) was added. The reaction mixture was stirred at 90° C. for 3 hours. After completion, the reaction mixture was cooled to room temperature, filtered through celite bed and washed with ethyl acetate (18 mL). The filtrate was diluted with water (50 ml), layers were separated and aqueous layer was extracted with ethyl acetate (2×10 mL). The combined organic layer was dried over anhydrous sodium sulfate, filtered and evaporated under reduced pressure to give crude product as a brown gum.

The crude product was pre-absorbed on silica, loaded on the pre-packed Orochem (25 g) column and eluted at 0-100% of ethyl acetate in pet ether with flow rate 25 ml/min. The pure product was eluted at 15-25% of ethyl acetate in pet ether. The appropriate fractions were collected and concentrated under reduced pressure to afford methyl 2′-chloro-5′-methoxy-[3,4′-bipyridine]-4-carboxylate (900 mg, 3.05 mmol, 65.8% yield) as white solid.

MS (ESI) calculated for (C₁₃H₁₁ClN₂O₃) (M+1)⁺, 279.06. found, 279.0.

### Step-2: Synthesis of 2′-chloro-5′-methoxy-[3,4′-bipyridine]-4-carboxylic Acid

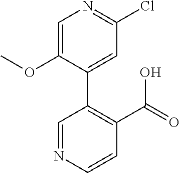 [SMILES: COc1cnc(Cl)cc1-c1cnccc1C(=O)O]

To a stirred solution of methyl 2′-chloro-5′-methoxy-[3,4′-bipyridine]-4-carboxylate (900 mg, 3.23 mmol) in a mixture tetrahydrofuran (10 mL), methanol (10 mL) and water (1 mL), was added lithium hydroxide monohydride (155 mg, 6.46 mmol) at room temperature. The reaction mixture was stirred at room temperature for 2 hours. After completion, The reaction mixture was concentrated under reduced pressure. The residue was diluted with water (1 mL) and neutralize with 1(N) HCl (8 mL). The precipitate formed was filtered through buchner funnel and washed water (2 mL), dried under vacuum to afford 2′-chloro-5′-methoxy-[3,4′-bipyridine]-4-carboxylic acid (600 mg, 2.154 mmol, 66.7% yield) as a white solid.

MS (ESI) calculated for (C₁₂H₉ClN₂O₃) (M+1)⁺, 265.04. found 265.0.

### Step-3: Synthesis of 2′-chloro-5′-methoxy-N-(5-methoxy-1,3,4-thiadiazol-2-yl)-[3,4′-bipyridine]-4-carboxamide

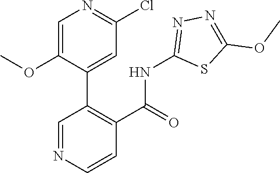

To a stirred solution of 2′-chloro-5′-methoxy-[3,4′-bipyridine]-4-carboxylic acid (150 mg, 0.567 mmol) in acetonitrile (3 mL) and N,N-dimethylformamide (0.6 mL) were added chloro-N,N,N′,N′-tetramethylformamidinium hexafluorophosphate (175 mg, 0.623 mmol), 1-methyl-1H-imidazole (140 mg, 1.7 mmol) and 5-methoxy-1,3,4-thiadiazol-2-amine (74.3 mg, 0.567 mmol) at room temperature. The reaction mixture was stirred at room temperature for 3 hours. After completion, The reaction mixture was quenched with ice cold water (10 mL), precipitated solid was filtered through buchner funnel, washed with 1:1 acetonitrile and water (2 mL), dried under vacuum to give crude product as white solid.

The crude product was purified by GRACE revelleris X2 (reverse phase) with the following condition: (Column: 100 g Grace C18 catriage; Mobile Phase A: 0.1% formic acid in water,

Mobile Phase B: acetonitrile) 0-100% of B in A for 40 minutes with flow rate 18 mL/min. The appropriate fractions were combined and evaporated in vacuum to afford 2′-chloro-5′-methoxy-N-(5-methoxy-1,3,4-thiadiazol-2-yl)-[3,4′-bipyridine]-4-carboxamide (75 mg, 0.197 mmol, 34.8% yield) as white solid.

MS (ESI) calculated for (C₁₅H₁₂ClN₅O₃S) (M+1)⁺, 378.05. found, 378.0.

1H-NMR (400 MHz, DMSO-d⁶): δ 13.05 (s, 1H), 8.84 (d, J=4.80 Hz, 1H), 8.72 (s, 1H), 8.16 (s, 1H), 7.76 (d, J=4.80 Hz, 1H), 7.61 (s, 1H), 4.08 (s, 3H), 3.62 (s, 3H).

### Example 272

### 4-(5-chloro-2-methoxy-4-(methylsulfonyl)phenyl)-N-(5-methoxy-1,3,4-thiadiazol-2-yl)-6-methylnicotinamide

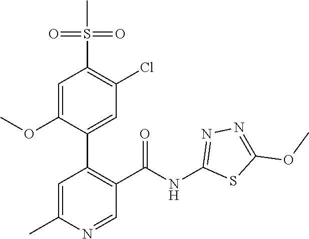

### Step-1: Synthesis of 1-bromo-5-chloro-2-methoxy-4-(methylsulfonyl)benzene

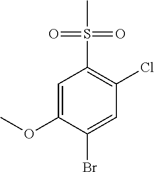

To a stirred solution of (4-bromo-2-chloro-5-methoxyphenyl)(methyl)sulfane (1.1 g, 4.11 mmol) in Dichloromethane (5 mL) was added 3-chloroperbenzoic acid (mCPBA) (1.774 g, 10.28 mmol) portion wise at 0° C., under nitrogen atmosphere. The resulting mixture was stirred at room temperature for 16 h. The reaction mixtures was diluted with 10% Na₂CO₃ aq. solution (25 mL) and extracted with ethyl acetate (2×25 mL), washed with brine. The organic layer was separated and dried over anhydrous sodium sulfate, concentrated under vacuum to afford 1-bromo-5-chloro-2-methoxy-4-(methylsulfonyl)benzene (1.17 g, 3.91 mmol, 95% yield) as yellow solid.

MS (ESI) calculated for (C₈H₈BrClO₃S) (M+1)⁺, 298.92. found, No ionization.

1H-NMR (400 MHz, DMSO-d⁶): δ 8.08 (s, 1H), 7.58 (s, 1H), 3.97 (s, 3H), 3.38 (s, 3H).

### Step-2: Synthesis of methyl 4-(5-chloro-2-methoxy-4-(methylsulfonyl)phenyl)-6-methylnicotinate

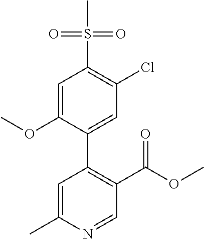

To a stirred solution of 1-bromo-5-chloro-2-methoxy-4-(methylsulfonyl)benzene (500 mg, 1.669 mmol) in 1,4-dioxane (5 mL) and water (1 mL) were added methyl 4-(5,5-dimethyl-1,3,2-dioxaborinan-2-yl)-6-methylnicotinate (527 mg, 2.003 mmol) and potassium carbonate (692 mg, 5.01 mmol) at room temperature and degassed with nitrogen for 10 minutes. To the resulting reaction mixture was added PdCl2(dppf) (122 mg, 0.167 mmol). The reaction mixture was stirred at 90° C. for 16 hours. After completion, the reaction mixture was filtered through celite bed and washed with Ethyl acetate (125 mL). The filtrate was washed with water (100 mL) and brine solution (100 mL). The organic layer was dried over anhydrous sodium sulfate, filtered and concentrated under reduced pressure to give the crude product as a brown liquid.

The crude product was pre-absorbed on silica, loaded on the pre-pack orochem 25 g column and eluted at 0-100% of ethyl acetate in pet ether with flow rate 25 ml/min. The pure product was eluted at 44% of ethyl acetate in pet ether. The appropriate fractions were collected and concentrated under reduced pressure to afford methyl 4-(5-chloro-2-methoxy-4-(methylsulfonyl)phenyl)-6-methylnicotinate (240 mg, 0.6 mmol) as a yellow oil.

MS (ESI) calculated for (C₁₆H₁₆ClNO₅S) (M+1)⁺, 370.05. found, 370.0.

### Step-3: Synthesis of 4-(5-chloro-2-methoxy-4-(methylsulfonyl)phenyl)-6-methylnicotinic Acid

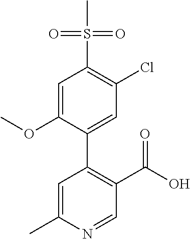 [SMILES: COc1cc(S(C)(=O)=O)c(Cl)cc1-c1cc(C)ncc1C(=O)O]

To a stirred solution of methyl 4-(5-chloro-2-methoxy-4-(methylsulfonyl)phenyl)-6-methylnicotinate (240 mg, 0.649 mmol) in a mixture of Tetrahydrofuran (4 mL), methanol (1.3 mL) and Water (1.3 mL) was added lithium hydroxide (46.6 mg, 1.947 mmol) at 0° C. The reaction mixture was stirred at room temperature for 16 hours. After completion of the reaction, the reaction mixture was concentrated under vacuum. The resulting residue diluted with water (20 mL) and washed with dichloromethane (2×30 mL). The aqueous layer was acidified with saturated solution of citric acid up to pH-6 and extracted with ethyl acetate (2×40 mL). The combined organic layer dried over anhydrous sodium sulfate, filtered and concentrated under reduced pressure to afford 4-(5-chloro-2-methoxy-4-(methylsulfonyl)phenyl)-6-methylnicotinic acid (126 mg, 0.346 mmol, 53.4% yield) as off-white solid.

MS (ESI) calculated for (C₁₅H₁₄ClNO₅S) (M+1)⁺, 356.04. found, 356.0.

### Step-4: Synthesis of 4-(5-chloro-2-methoxy-4-(methylsulfonyl)phenyl)-N-(5-methoxy-1,3,4-thiadiazol-2-yl)-6-methylnicotinamide

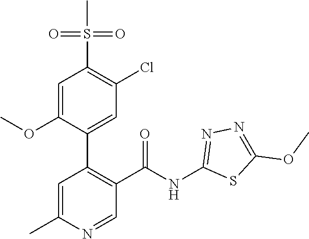

To a stirred solution of 4-(5-chloro-2-methoxy-4-(methylsulfonyl)phenyl)-6-methylnicotinic acid (50 mg, 0.141 mmol), in acetonitrile (2 mL) and N,N-Dimethylformamide (0.4 mL) were added 1-methyl-1H-imidazole (0.045 mL, 0.562 mmol), chloro-N,N,N′,N′-tetramethylformamidinium hexafluorophosphate (59.1 mg, 0.211 mmol), and 5-methoxy-1,3,4-thiadiazol-2-amine (18.43 mg, 0.141 mmol) at room temperature. The reaction mixture was stirred at room temperature for 16 hours. After completion the reaction mixture was concentrated under reduced pressure. The residue was diluted with water and extracted with ethyl acetate (2×5 mL). The combined organic layer dried over anhydrous sodium sulfate, filtered and concentrated under vacuum to afford crude product.

The crude product was pre-absorbed on silica, loaded on the pre-pack orochem 12 g column and eluted at 0-10% of methanol in dichloromethane with flow rate 20 ml/min. The appropriate fractions were collected and concentrated under reduced pressure to afford 4-(5-chloro-2-methoxy-4-(methylsulfonyl)phenyl)-N-(5-methoxy-1,3,4-thiadiazol-2-yl)-6-methylnicotinamide (25 mg, 0.053 mmol, 37.8% yield) as off white solid.

MS (ESI) calculated for (C₁₈H₁₇ClN₄O₅S₂) (M+1)⁺, 469.04. found, 469.1.

1H-NMR (400 MHz, DMSO-d⁶): δ 12.93 (s, 1H), 8.79 (s, 1H), 7.77 (s, 1H), 7.50 (s, 1H), 7.42 (s, 1H), 4.08 (s, 3H), 3.61 (s, 3H), 3.45 (s, 3H), 2.59 (s, 3H).

### Example 321 and 318

### (R)-4-(5-chloro-4-(cyclopropylsulfinyl)-2-methoxyphenyl)-N-(5-methoxy-1,3,4-thiadiazol-2-yl)-6-methylnicotinamide and (S)-4-(5-chloro-4-(cyclopropylsulfinyl)-2-methoxyphenyl)-N-(5-methoxy-1,3,4-thiadiazol-2-yl)-6-methylnicotinamide

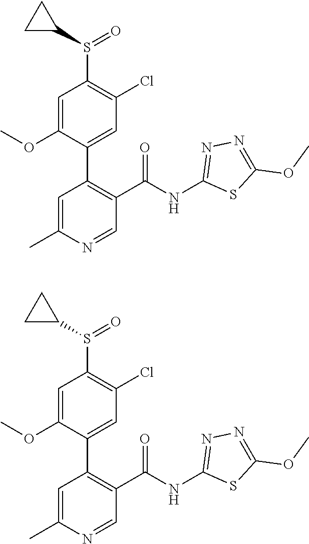

### Step-1: Synthesis of 4-bromo-2-chloro-5-methoxy Benzenesulfonyl Chloride

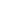

Thionyl chloride (6.17 mL, 85 mmol) was added to Water (30 mL) at −5° C. followed by copper(I) chloride (0.042 g, 0.423 mmol) and resulting reaction mixture was stirred at room temperature for 5 hours. In a separate flask, a 4-bromo-2-chloro-5-methoxyaniline (2.0 g, 8.46 mmol) in acetonitrile (20 mL) was added HCl (12 M) (1.409 mL, 16.91 mmol) dropwise at 0° C., The mixture was stirred at 0° C. for 10 minutes followed by addition of sodium nitrite (0.875 g, 12.69 mmol) in Water (15 mL) dropwise 0° C. and stirred for 1 hour at same temperature. After 1 hour, Diazonium salt was added to the thionyl chloride solution at 0° C. and the resulting reaction mixture was stirred at 0° C. for 1 hour. After completion, the reaction mixture was diluted with dichloromethane (100 mL). The layers were separated and aqueous layer was extracted with dichloromethane (100 mL). The combined organic layer was dried over sodium sulfate, filtered and concentrated under reduced pressure to afford 4-bromo-2-chloro-5-methoxybenzenesulfonyl chloride (2.7 g) as a red gum. The crude was forwarded for next step without purification.

MS (ESI) calculated for (C₇H₅BrCl₂O₃S) (M+1)⁺, 318.86. found, No ionization.

1H-NMR (400 MHz, DMSO-d⁶): δ 7.60 (s, 1H), 7.55 (s, 1H), 3.85 (s, 3H).

### Step-2: Synthesis of 4-bromo-2-chloro-5-methoxybenzenethiol

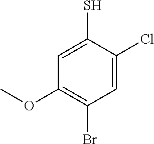

To a stirred solution of 4-bromo-2-chloro-5-methoxybenzenesulfonyl chloride (2.70 g, 8.44 mmol) in toluene (30 mL) was added triphenylphosphine (6.64 g, 25.3 mmol). The resulting reaction mixture was stirred at 110° C. for 30 minutes. After 30 minutes, reaction mixture was allowed to cool to 60° C., and water (6 mL) was added. The resulted mixture was stirred at 60° C. for additional 30 minutes. After completion, The reaction mixture was allowed to cool to room temperature, basified with 20% sodium hydroxide solution (25 mL). The resulted mixture was washed with toluene (100 mL). The aqueous layer was acidified with 1.5 N HCl to pH 2 and extracted with dichloromethane (2×50 mL). The combined dichloromethane layers were dried over sodium sulfate, filtered and concentrated under reduced pressure to afford 4-bromo-2-chloro-5-methoxybenzenethiol (1.10 g, 3.28 mmol, 38.8% yield; LCMS purity: 75.5%) as off-white solid.

MS (ESI) calculated for (C₇H₆BrClOS) (M−1)⁻, 252.89. found, 252.8.

### Step-3: Synthesis of (4-bromo-2-chloro-5-methoxyphenyl)(cyclopropyl)sulfane

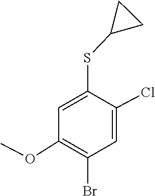

To a stirred solution of 4-bromo-2-chloro-5-methoxybenzenethiol (1.1 g, 3.28 mmol) in 1,2-Dichloroethane (20 mL), was added cyclopropylboronic acid (0.422 g, 4.92 mmol), 2,2′-bipyridine (0.512 g, 3.28 mmol) and copper (II) acetate (0.595 g, 3.28 mmol) at room temperature. The reaction mixture was stirred at 70° C. for 16 hours. After completion, the reaction mixture was cooled to room temperature, quenched with water (30 mL) and extracted with dichloromethane (2×50 mL). The combined organic layer was dried over anhydrous sodium sulfate, filtered and evaporated under reduced pressure to give crude product as a black gum.

The crude product was pre-absorbed on silica, loaded on the pre-packed Orochem (50 g) column and eluted at 0-100% of ethyl acetate in pet ether over 30 minutes with flow rate 20 ml/min. The pure product was eluted at 10% of ethyl acetate in pet ether. The appropriate fractions were collected and concentrated under reduced pressure to afford (4-bromo-2-chloro-5-methoxyphenyl)(cyclopropyl)sulfane (1.6 g, 5.41 mmol, 165% yield) as off white solid.

MS (ESI) calculated for (C₁₀H₁₀BrClOS) (M)⁺, 293.9. found, GCMS m/z 293.9.

### Step-4: Synthesis of 1-bromo-5-chloro-4-(cyclopropylsulfinyl)-2-methoxybenzene

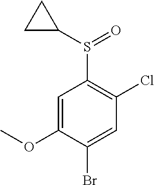

A solution of (4-bromo-2-chloro-5-methoxyphenyl)(cyclopropyl)sulfane (1.17 g, 3.98 mmol) in dichloromethane (20 mL), was cooled to 0° C. then meta-chloroperoxy benzoic acid (m-CPBA) (0.688 g, 3.98 mmol) was added portion wise at 0° C. The reaction mixture was stirred for 1 hours at room temperature. After completion, the reaction mixture was quenched with sat. sodium bicarbonate solution (20 mL) and extracted with dichloromethane (2×50 mL). The organic layer was separated, dried over anhydrous sodium sulfate and concentrated under vacuum to afford crude product as colorless gum.

The crude product was pre-absorbed on silica, loaded on the pre-packed Orochem (50 g) column and eluted at 0-100% of ethyl acetate in pet ether over 50 minutes with flow rate 20 ml/min. The pure product was eluted at 80% of ethyl acetate in pet ether. The appropriate fractions were collected and concentrated under reduced pressure to afford 1-bromo-5-chloro-4-(cyclopropylsulfinyl)-2-methoxybenzene (1.2 g, 3.71 mmol, 93% yield) as off white solid.

MS (ESI) calculated for (C₁₀H₁₀BrClO₂S) (M+1)⁺, 308.94. found, 309.0.

### Step-5: Synthesis of methyl 4-(5-chloro-4-(cyclopropylsulfinyl)-2-methoxyphenyl)-6-methylnicotinate

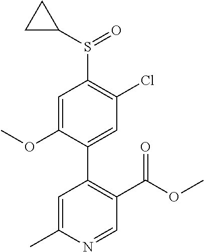

To a stirred solution of 1-bromo-5-chloro-4-(cyclopropylsulfinyl)-2-methoxybenzene (1.0 g, 3.23 mmol) in 1,4-dioxane (10 mL) and water (4 mL) was added methyl 4-(5,5-dimethyl-1,3,2-dioxaborinan-2-yl)-6-methylnicotinate (1.530 g, 5.81 mmol), potassium carbonate (1.339 g, 9.69 mmol) at room temperature. Then the reaction mixture was degassed by nitrogen gas for 10 minutes. PdCl2(dppf) (0.236 g, 0.323 mmol) was added. The reaction mixture was stirred at 90° C. for 16 hours. After completion, the reaction mixture was quenched with water (10 mL) and extracted with ethyl acetate (2×50 mL). The combined organic layer was dried over anhydrous sodium sulfate, filtered and evaporated under reduced pressure to give crude product as a black gum.

The crude product was pre-absorbed on silica, loaded on the pre-packed Orochem (50 g) column and eluted at 0-100% of ethyl acetate in pet ether over 50 minutes with flow rate 30 ml/min. The pure product was eluted at 80% of ethyl acetate in pet ether. The appropriate fractions were collected and concentrated under reduced pressure to afford product methyl 4-(5-chloro-4-(cyclopropylsulfinyl)-2-methoxyphenyl)-6-methylnicotinate (0.8 g, 2.027 mmol, 62.8% yield) as off-white solid.

MS (ESI) calculated for (C₁₈H₁₈ClNO₄S) (M+1)⁺, 380.08. found, 380.0.

### Step-6: Synthesis of 4-(5-chloro-4-(cyclopropylsulfinyl)-2-methoxyphenyl)-6-methylnicotinic Acid

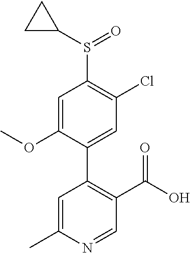 [SMILES: COc1cc(S(=O)C2CC2)c(Cl)cc1-c1cc(C)ncc1C(=O)O]

To a stirred solution of methyl 4-(5-chloro-4-(cyclopropylsulfinyl)-2-methoxyphenyl)-6-methylnicotinate (0.8 g, 2.106 mmol) in a mixture methanol (15 mL), and water (5 mL), was added lithium hydroxide monohydrate (0.252 g, 10.53 mmol) at room temperature. The reaction mixture was stirred at room temperature for 16 hours. After completion, The reaction mixture was concentrated under reduced pressure. The residue was diluted with water (10 mL) and neutralized with 1.5(N) HCl. The aqueous layer was purified by GRACE revelleris X2 (reverse phase) with the following condition: (Column: 50 g Grace C18 catriage; Mobile Phase A: 0.1% formic acid in water, Mobile Phase B: acetonitrile) 0-100% of B in A for 30 minutes with flow rate 20 mL/min, the pure product was eluted at 55% of B in A. The appropriate fractions were combined and evaporated in vacuum to afford 4-(5-chloro-4-(cyclopropylsulfinyl)-2-methoxyphenyl)-6-methylnicotinic acid (0.7 g, 1.907 mmol, 91% yield) as off-white solid.

MS (ESI) calculated for (C₁₇H₁₆ClNO₄S) (M+1)⁺, 366.05. found 366.0.

### Step-7: Synthesis of 4-(5-chloro-4-(cyclopropylsulfinyl)-2-methoxyphenyl)-N-(5-methoxy-1,3,4-thiadiazol-2-yl)-6-methylnicotinamide

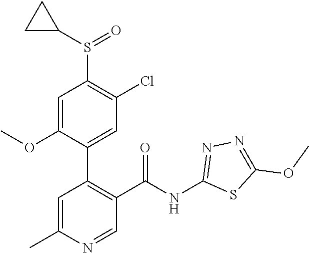

To a stirred solution of 4-(5-chloro-4-(cyclopropylsulfinyl)-2-methoxyphenyl)-6-methylnicotinic acid (150 mg, 0.410 mmol) in acetonitrile (5 mL) and N,N-dimethylformamide (1 mL) were added, 1-methyl-1H-imidazole (0.131 mL, 1.640 mmol), chloro-N,N,N′,N′-tetramethylformamidinium hexafluorophosphate (173 mg, 0.615 mmol) and 5-methoxy-1,3,4-thiadiazol-2-amine (64.5 mg, 0.492 mmol) at room temperature. stirred at room temperature for 16 hours. After completion The reaction mixture was quenched with water (2 mL) and purified by GRACE revelleris X2 (reverse phase) with the following condition: (Column: 50 g Grace C18 catriage; Mobile Phase A: 0.1% formic acid in water, Mobile Phase B: acetonitrile) 0-100% of B in A with flow rate 25 mL/min, the pure product was eluted at 40% of B in A. The appropriate fractions were combined and evaporated in vacuum to afford 4-(5-chloro-4-(cyclopropylsulfinyl)-2-methoxyphenyl)-N-(5-methoxy-1,3,4-thiadiazol-2-yl)-6-methylnicotinamide (125 mg, 0.258 mmol, 62.9% yield) as off-white solid.

MS (ESI) calculated for (C₂₀H₁₉ClN₄O₄S₂) (M+1)⁺, 479.06. found, 479.0.

1H-NMR (400 MHz, DMSO-d⁶): δ 12.83 (s, 1H), 8.74 (s, 1H), 7.63 (s, 1H), 7.43 (s, 1H), 7.20 (s, 1H), 4.07 (s, 3H), 3.59 (s, 3H), 2.74-2.67 (m, 1H), 2.58 (s, 3H), 1.08-0.95 (m, 3H), 0.80-0.72 (m, 1H).

### Step 8: Chiral separation of (R)-4-(5-chloro-4-(cyclopropylsulfinyl)-2-methoxyphenyl)-N-(5-methoxy-1,3,4-thiadiazol-2-yl)-6-methylnicotinamide and (S)-4-(5-chloro-4-(cyclopropylsulfinyl)-2-methoxyphenyl)-N-(5-methoxy-1,3,4-thiadiazol-2-yl)-6-methylnicotinamide

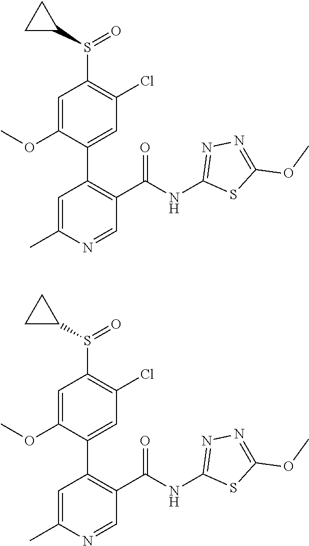

4-(5-Chloro-4-(cyclopropylsulfinyl)-2-methoxyphenyl)-N-(5-methoxy-1,3,4-thiadiazol-2-yl)-6-methylnicotinamide (160 mg) was separated by prep-chiral SFC with the following condition: (Column: Lux Amylose-1 (250*30) mm, 5 μm; Mobile Phase A: CO₂, Mobile Phase B: Methanol; Gradient: isocratic 40% B; Column Temperature (° C.): 35; Back Pressure (bar): 100; Wave Length: 220 nm; RT1 (min): 2.16; RT2 (min): 3.52; (Sample Solvent: 3 mL MeOH-HPLC; Injection Volume: 0.4 mL/Injection; Cycle time: 9 min) to afford (R)-4-(5-chloro-4-(cyclopropylsulfinyl)-2-methoxyphenyl)-N-(5-methoxy-1,3,4-thiadiazol-2-yl)-6-methylnicotinamide (70 mg, 0.146 mmol, 43.7% yield) as off-white solid with the first peak on chiral SFC with shorter retention time and (S)-4-(5-chloro-4-(cyclopropylsulfinyl)-2-methoxyphenyl)-N-(5-methoxy-1,3,4-thiadiazol-2-yl)-6-methylnicotinamide (65 mg, 0.134 mmol, 40.1% yield) as off-white solid with the second peak on chiral SFC with longer retention time.

The absolute stereochemistry was not determined.

### (R)-4-(5-chloro-4-(cyclopropylsulfinyl)-2-methoxyphenyl)-N-(5-methoxy-1,3,4-thiadiazol-2-yl)-6-methylnicotinamide

MS (ESI) calculated for (C₂₀H₁₉ClN₄O₄S₂) (M+1)⁺, 479.06. found, 478.8.

1H-NMR (400 MHz, DMSO-d⁶): δ 12.84 (s, 1H), 8.76 (s, 1H), 7.61 (s, 1H), 7.41 (s, 1H), 7.20 (s, 1H), 4.06 (s, 3H), 3.59 (s, 3H), 2.74-2.67 (m, 1H), 2.58 (s, 3H), 1.08-0.95 (m, 3H), 0.80-0.72 (m, 1H).

### (S)-4-(5-bromo-2-methoxy-4-(methylsulfinyl)phenyl)-N-(5-methoxy-1,3,4-thiadiazol-2-yl)-6-methylnicotinamide

MS (ESI) calculated for (C₂₀H₁₉ClN₄O₄S₂) (M−1)⁻, 477.04. found, 477.0.

1H-NMR (400 MHz, DMSO-d⁶): δ 12.83 (s, 1H), 8.75 (s, 1H), 7.63 (s, 1H), 7.43 (s, 1H), 7.20 (s, 1H), 4.07 (s, 3H), 3.59 (s, 3H), 2.74-2.67 (m, 1H), 2.58 (s, 3H), 1.08-0.94 (m, 3H), 0.80-0.71 (m, 1H).

### Example 320

### 4-(6-chloro-3,4-dihydro-2H-benzo[b][1,4]oxazin-8-yl)-N-(5-methoxy-1,3,4-thiadiazol-2-yl)-6-methylnicotinamide

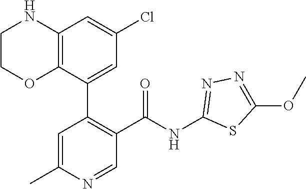 [SMILES: COc1nnc(NC(=O)c2cnc(C)cc2-c2cc(Cl)cc3c2OCCN3)s1]

### Step-1: Synthesis of 2-bromo-4-chloro-6-nitrophenol

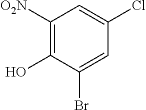 [SMILES: O=[N+]([O-])c1cc(Cl)cc(Br)c1O]

A solution of 4-chloro-2-nitrophenol (10 g, 57.6 mmol) in Acetic Acid (80 mL) was cooled to 5-10° C. and bromine (3.56 mL, 69.1 mmol) was added dropwise. The reaction mixture was stirred for 30 minutes at 10° C. and then for 16 hour at room temperature. After completion, The reaction mixture was quenched with ice cold water (220 mL) and extracted with dichloromethane (2×30 mL). The combined organic phase was washed with 10% sodium bicarbonate solution (100 mL), water (100 mL) and brine (100 mL), dried over anhydrous sodium sulfate, filtered and concentrated under reduced pressure to afford 2-bromo-4-chloro-6-nitrophenol (14 g, 49.1 mmol, 85% yield) as yellow solid.

MS (ESI) calculated for (C₆H₃BrClNO₃) (M−1)⁻, 249.89. found, 249.8.

### Step-2: Synthesis of 1-bromo-2-(2-bromoethoxy)-5-chloro-3-nitrobenzene

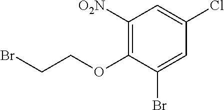 [SMILES: O=[N+]([O-])c1cc(Cl)cc(Br)c1OCCBr]

To a stirred solution of 2-bromo-4-chloro-6-nitrophenol (14 g, 49.1 mmol) in N,N-dimethylformamide (100 mL) was added portion wise potassium carbonate (20.35 g, 147 mmol), followed by 1,2-dibromoethane (21.15 mL, 245 mmol). The reaction mixture was stirred at 80° C. for 5 hours. After completion, the reaction mixture was quenched with ice cold water (200 mL) and extracted with ethyl acetate (2×200 mL). The combined organic layer was washed with water (100 mL), brine (100 mL), dried over sodium sulfate, filtered and concentrated under reduced pressure to give crude product as brown solid.

The crude product was pre-absorbed on silica, loaded on pre-packed Orochem (125 g) column and eluted at 0-10% of ethyl acetate in pet ether with flow rate 20 ml/min. The pure product was eluted at 4-6% of ethyl acetate in pet ether. The appropriate fractions were collected and concentrated under reduced pressure to afford 1-bromo-2-(2-bromoethoxy)-5-chloro-3-nitrobenzene (14.2 g, 36.7 mmol, 74.8% yield) as yellow solid.

MS (ESI) calculated for (C₈H₆Br₂ClNO₃) (M+1)⁺, 329.6. found, 329.8.

### Step-3: Synthesis of 3-bromo-2-(2-bromoethoxy)-5-chloroaniline

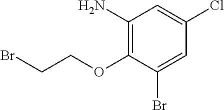 [SMILES: Nc1cc(Cl)cc(Br)c1OCCBr]

To a stirred solution of 1-bromo-2-(2-bromoethoxy)-5-chloro-3-nitrobenzene (14 g, 39.0 mmol)) in acetic acid (100 mL) was added iron (8.70 g, 156 mmol) portion wise at room temperature. The reaction mixture was stirred at room temperature for 16 hours. After completion, The reaction mixture was diluted with ethyl acetate (400 mL) and filtered through celite. The celite bed was washed with ethyl acetate (100 mL). The filtrate was diluted with water (200 mL) and layers were separated. The organic layer was washed with water, brine, dried over sodium sulfate, filtered and concentrated under reduced pressure to afford 3-bromo-2-(2-bromoethoxy)-5-chloroaniline (12.1 g, 29.7 mmol, 76% yield; LCMS purity: 80.8%) as brown Liquid.

MS (ESI) calculated for (C₈H₈Br₂ClNO) (M)⁺, 329.42. found, 329.8.

### Step-4: Synthesis of 8-bromo-6-chloro-3,4-dihydro-2H-benzo[b][1,4]oxazine

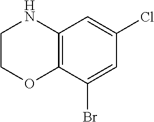 [SMILES: Clc1cc(Br)c2c(c1)NCCO2]

To a mixture of 3-bromo-2-(2-bromoethoxy)-5-chloroaniline (12 g, 29.4 mmol) in N,N-Dimethylformamide (120 mL) was added potassium carbonate (18.31 g, 132 mmol) portion wise at room temperature. The reaction was stirred at 80° C. for 3 hours. After completion, the reaction mixture was quenched with ice cold water (200 mL) and extracted with ethyl acetate (2×200 mL). The combined organic layer was washed with water, brine, dried over sodium sulfate, filtered and concentrated under reduced pressure to give crude product as brown liquid.

The crude product was pre-absorbed on silica, loaded on pre-packed Orochem (125 g) column and eluted at 0-10% of ethyl acetate in pet ether with flow rate 20 ml/min. The pure product was eluted at 15-18% of ethyl acetate in pet ether. The appropriate fractions were collected and concentrated under reduced pressure to afford 8-bromo-6-chloro-3,4-dihydro-2H-benzo[b][1,4]oxazine (7.1 g, 28.3 mmol, 96% yield) as brown gum.

MS (ESI) calculated for (C₈H₇BrClNO) (M+2)⁺, 250.52. found, 250.0.

### Step-5: Synthesis of tert-butyl 8-bromo-6-chloro-2,3-dihydro-4H-benzo[b][1,4]oxazine-4-carboxylate

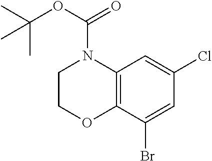 [SMILES: CC(C)(C)OC(=O)N1CCOc2c(Br)cc(Cl)cc21]

To a stirred solution of 8-bromo-6-chloro-3,4-dihydro-2H-benzo[b][1,4]oxazine (3 g, 12.07 mmol) in tetrahydrofuran (45 mL) were added DMAP (0.737 g, 6.04 mmol) and Boc-anhydride (3.36 mL, 14.49 mmol) at 0° C. The resulting reaction mixture was stirred at room temperature for 16 hours. After completion, the reaction mixture was quenched with ice cold water (50 mL) and extracted with ethyl acetate (2×30 mL). The combined organic layer was washed with water (50 mL) and brine (50 mL), dried over anhydrous sodium sulfate, filtered and concentrated under reduced pressure to give crude product as brown liquid.

The crude product was pre-absorbed on silica, loaded on pre-packed Orochem (125 g) column and eluted at 0-50% of ethyl acetate in pet ether with flow rate 20 ml/min. The pure product was eluted at 8-10% of ethyl acetate in pet ether. The appropriate fractions were collected and concentrated under reduced pressure to afford tert-butyl 8-bromo-6-chloro-2,3-dihydro-4H-benzo[b][1,4]oxazine-4-carboxylate (3.1 g, 8.87 mmol, 73.5% yield) as off-white solid.

MS (ESI) calculated for (C₁₃H₁₅BrClNO₃) (M−100)⁻, 248. found, 247.8.

1H-NMR (400 MHz, DMSO-d⁶): δ 7.90 (brs, 1H), 7.41 (d, J=2.4 Hz, 1H), 4.33 (t, J=4.4 Hz 2H), 3.83 (t, J=4.4 Hz 2H), 1.50 (s, 9H).

### Step-6: Synthesis of tert-butyl 6-chloro-8-(5-(methoxycarbonyl)-2-methylpyridin-4-yl)-2,3-dihydro-4H-benzo[b][1,4]oxazine-4-carboxylate

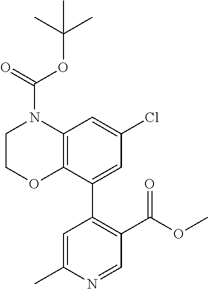 [SMILES: COC(=O)c1cnc(C)cc1-c1cc(Cl)cc2c1OCCN2C(=O)OC(C)(C)C]

To a stirred solution of tert-butyl 8-bromo-6-chloro-2,3-dihydro-4H-benzo[b][1,4]oxazine-4-carboxylate (1.5 g, 4.30 mmol) in 1,4-dioxane (10 mL) and water (0.5 mL), were added methyl 4-(5,5-dimethyl-1,3,2-dioxaborinan-2-yl)-6-methylnicotinate (1.698 g, 6.45 mmol), potassium carbonate (1.784 g, 12.91 mmol) at room temperature. Then the reaction mixture was degassed by nitrogen gas for 5 minutes. PdCl2(dppf) (0.315 g, 0.430 mmol) was added. The reaction mixture was stirred at 90° C. for 16 hours. After completion, the reaction mixture was cooled to room temperature, filtered through celite bed and washed with ethyl acetate (50 mL). The filtrate was diluted with water (50 ml), layers were separated and aqueous layer was extracted with ethyl acetate (50 mL). The combined organic layer was dried over anhydrous sodium sulfate, filtered and evaporated under reduced pressure to give crude product as a brown liquid.

The crude product was pre-absorbed on silica, loaded on the pre-packed Orochem (125 g) column and eluted at 0-100% of ethyl acetate in pet ether with flow rate 20 ml/min. The pure product was eluted at 42% of ethyl acetate in pet ether. The appropriate fractions were collected and concentrated under reduced pressure to afford tert-butyl 6-chloro-8-(5-(methoxycarbonyl)-2-methylpyridin-4-yl)-2,3-dihydro-4H-benzo[b][1,4]oxazine-4-carboxylate (1.7 g, 3.99 mmol, 93% yield) as yellow gum.

MS (ESI) calculated for (C₂₁H₂₃ClN₂O₅) (M+1)⁺, 419.14. found, 419.0.

### Step-7: Synthesis of 4-(4-(tert-butoxycarbonyl)-6-chloro-3,4-dihydro-2H-benzo[b][1,4]oxazin-8-yl)-6-methylnicotinic Acid

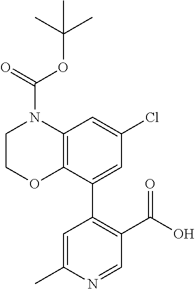 [SMILES: Cc1cc(-c2cc(Cl)cc3c2OCCN3C(=O)OC(C)(C)C)c(C(=O)O)cn1]

To a stirred solution of tert-butyl 6-chloro-8-(5-(methoxycarbonyl)-2-methylpyridin-4-yl)-2,3-dihydro-4H-benzo[b][1,4]oxazine-4-carboxylate (1.7 g, 4.06 mmol) in a mixture tetrahydrofuran (20 mL), and water (6.67 mL), was added lithium hydroxide monohydrate (0.852 g, 20.29 mmol) at room temperature. The reaction mixture was stirred at room temperature for 16 hours. After completion, The reaction mixture was concentrated under reduced pressure. The residue was diluted with water (20 mL) and neutralized with 1.5(N) HCl (15 mL) and extracted with 10% methanol in dichloromethane (2×30 mL). The combined organic layer was dried over anhydrous sodium sulfate, filtered and evaporated under reduced pressure to afford 4-(4-(tert-butoxycarbonyl)-6-chloro-3,4-dihydro-2H-benzo[b][1,4]oxazin-8-yl)-6-methylnicotinic acid (1.2 g, 2.87 mmol, 70.7% yield) as a yellow solid.

MS (ESI) calculated for (C₂₀H₂₁ClN₂O₅) (M+1)⁺, 405.12. found 405.0.

### Step-8: Synthesis of tert-butyl 6-chloro-8-(5-((5-methoxy-1,3,4-thiadiazol-2-yl)carbamoyl)-2-methylpyridin-4-yl)-2,3-dihydro-4H-benzo[b][1,4]oxazine-4-carboxylate

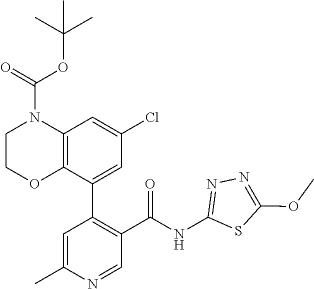 [SMILES: COc1nnc(NC(=O)c2cnc(C)cc2-c2cc(Cl)cc3c2OCCN3C(=O)OC(C)(C)C)s1]

To a stirred solution of 4-(4-(tert-butoxycarbonyl)-6-chloro-3,4-dihydro-2H-benzo[b][1,4]oxazin-8-yl)-6-methylnicotinic acid (300 mg, 0.741 mmol) in acetonitrile (3 mL) and N,N-dimethylformamide (075 mL) were added 5-methoxy-1,3,4-thiadiazol-2-amine (97 mg, 0.741 mmol), 1-methyl-1H-imidazole (0.177 mL, 2.223 mmol) and chloro-N,N,N′,N′-tetramethylformamidinium hexafluorophosphate (312 mg, 1.112 mmol) at room temperature. The reaction mixture was stirred at room temperature for 16 hours. After completion, the reaction mixture was quenched with ice cold water (50 mL) and extracted with ethyl acetate (2×30 mL). The combined organic layer was washed with water (40 mL), brine (40 mL), dried over anhydrous sodium sulfate, filtered and concentrated under reduced pressure to give crude product as brown liquid.

The crude product was pre-absorbed on silica, loaded on the pre-packed Orochem (25 g) column and eluted at 0-10% of methanol in dichloromethane with flow rate 30 ml/min. The pure product was eluted at 6-8% of methanol in dichloromethane. The appropriate fractions were collected and concentrated under reduced pressure to afford tert-butyl 6-chloro-8-(5-((5-methoxy-1,3,4-thiadiazol-2-yl)carbamoyl)-2-methylpyridin-4-yl)-2,3-dihydro-4H-benzo[b][1,4]oxazine-4-carboxylate (110 mg, 0.202 mmol, 27.3% yield) as off-white solid

MS (ESI) calculated for (C₂₃H₂₄ClN₅O₅S) (M+1)⁺, 518.13. found, 518.0.

### Step-9: 4-(6-chloro-3,4-dihydro-2H-benzo[b][1,4]oxazin-8-yl)-N-(5-methoxy-1,3,4-thiadiazol-2-yl)-6-methylnicotinamide

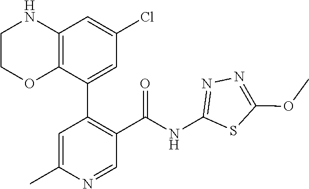 [SMILES: COc1nnc(NC(=O)c2cnc(C)cc2-c2cc(Cl)cc3c2OCCN3)s1]

To a stirred solution of tert-butyl 6-chloro-8-(5-((5-methoxy-1,3,4-thiadiazol-2-yl)carbamoyl)-2-methylpyridin-4-yl)-2,3-dihydro-4H-benzo[b][1,4]oxazine-4-carboxylate (110 mg, 0.212 mmol) in dichloromethane (3 mL) was added trifluoroacetic acid (0.082 mL, 1.062 mmol) at 0° C. The reaction mixture was stirred at room temperature for 13 hours. After completion, The reaction mixture was quenched with ice cold water (20 mL) at room temperature and extracted with 10% methanol in dichloromethane (2×20 mL). The combined organic layer was washed with water (20 mL), brine (30 mL), dried over anhydrous sodium sulfate, filtered and concentrated under reduced pressure to give crude product as brown solid.

The crude product was purified by Isolera biotage (reverse phase) with the following condition: (Column: 30 g Grace C18 Redisep Gold; Mobile Phase A: water, Mobile Phase B: acetonitrile; neutral method) 0-100% of B in A for 30 minutes with flow rate 25 mL/min. The appropriate fractions were combined and evaporated in vacuum to afford 4-(6-chloro-3,4-dihydro-2H-benzo[b][1,4]oxazin-8-yl)-N-(5-methoxy-1,3,4-thiadiazol-2-yl)-6-methylnicotinamide (51 mg, 0.118 mmol, 55.6% yield) as white solid.

MS (ESI) calculated for (C₁₈H₁₆ClN₅O₃S) (M+1)⁺, 418.08. found, 418.0.

1H-NMR (400 MHz, DMSO-d⁶): δ 12.75 (s, 1H), 8.66 (s, 1H), 7.33 (s, 1H), 6.62 (d, J=2.4 Hz 1H), 6.56 (d, J=2.4 Hz 1H), 6.19 (brs, 1H), 4.08 (s, 3H), 3.76 (brs, 2H), 3.14 (t, J=4.00 Hz, 2H), 2.56 (s, 3H).

### Example 319 and 322

### (R)-4-(5-bromo-2-methoxy-4-(methylsulfinyl)phenyl)-N-(5-methoxy-1,3,4-thiadiazol-2-yl)-6-methylnicotinamide and (S)-4-(5-bromo-2-methoxy-4-(methylsulfinyl)phenyl)-N-(5-methoxy-1,3,4-thiadiazol-2-yl)-6-methylnicotinamide

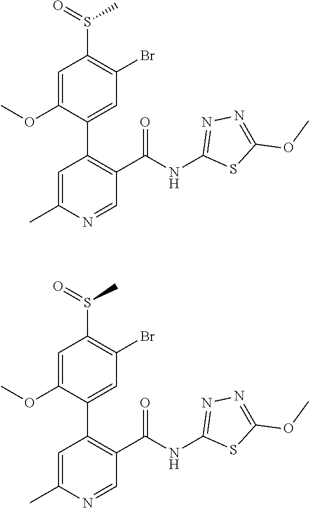

### Step-1: Synthesis of 2-bromo-4-iodo-5-methoxyaniline

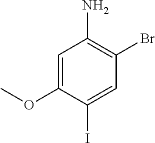

To a mixture of 2-bromo-5-methoxyaniline (2.5 g, 12.37 mmol) and Calcium carbonate (1.610 g, 16.09 mmol) in dichloromethane (50 mL) and methanol (25 mL) was added tetramethylammonium dichloroiodate(I) (3.70 g, 13.61 mmol) in one portion at room temperature. The reaction was stirred for 1 hour at room temperature. After completion, the reaction mixture was mixed with another batch (0.5 g) and diluted with dichloromethane (500 mL) washed with water (2×200 mL). The combined organic layer was dried over anhydrous sodium sulfate, filtered and concentrated under reduced pressure to give the crude product as black solid.

The crude product was pre-absorbed on silica, loaded on pre-packed Orochem (120 g) column and eluted at 0-10% of ethyl acetate in pet ether with flow rate 50 ml/min. The appropriate fractions were collected and concentrated under reduced pressure to afford product 2-bromo-4-iodo-5-methoxyaniline (4 g, 11.71 mmol, 95% yield) as brown solid.

MS (ESI) calculated for (C₇H₇BrINO) (M+2)⁺, 329.89. found, 329.8.

1H-NMR (400 MHz, DMSO-d⁶): δ 7.56 (s, 1H), 6.49 (s, 1H), 5.51 (brs, 2H), 3.72 (s, 3H).

### Step-2: Synthesis of (2-bromo-4-iodo-5-methoxyphenyl)(methyl)sulfane

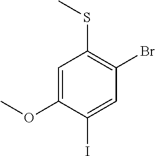

To a stirred solution of 2-bromo-4-iodo-5-methoxyaniline (4 g, 12.20 mmol) in 2N HCl hydrogen chloride (48.8 mL, 98 mmol), the reaction mixture was heated to 50° C. for 30 minutes, then the reaction mixture was cooled to 0° C., was added sodium nitrite (0.926 g, 13.42 mmol) in water (40 mL) drop wise and stirred for 40 minutes at 0° C., then sodium thiomethoxide (1.710 g, 24.39 mmol) in water (40 mL) was added dropwise at 0° C. The reaction mixture was stirred at room temperature for 18 hours. After completion, the reaction mixture was diluted in ethyl acetate (200 mL) and layers were separated. The organic layer dried over anhydrous sodium sulfate and concentrated under vacuum to afford crude product as dark brown oil.

The crude product was pre-absorbed on silica, loaded on pre-packed Orochem (120 g) column and eluted at 0-100% of ethyl acetate in pet ether with flow rate 50 ml/min. The pure product was eluted at 15-20% of ethyl acetate in pet ether. The appropriate fractions were collected and concentrated under reduced pressure to afford product (2-bromo-4-iodo-5-methoxyphenyl)(methyl)sulfane (3 g, 7.37 mmol, 60.4% yield) as brown solid.

MS (ESI) calculated for (C₈H₈BrIOS) (M)⁺, 357.85. found, GCMS m/z 357.8.

### Step-3: Synthesis of 1-bromo-5-iodo-4-methoxy-2-(methylsulfinyl)benzene

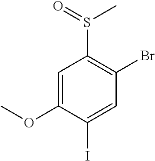

A solution of (2-bromo-4-iodo-5-methoxyphenyl)(methyl)sulfane (3 g, 8.36 mmol) in dichloromethane (30 mL), was cooled to 0° C. then meta-chloroperoxy benzoic acid (m-CPBA) (1.442 g, 8.36 mmol) was added portion wise at 0° C. The reaction mixture was stirred for 16 hours at room temperature. After completion, the reaction mixture was quenched with sat. sodium bicarbonate solution (50 mL) and extracted with dichloromethane (2×100 mL). The organic layer was separated, dried over anhydrous sodium sulfate and concentrated under vacuum to afford crude product as orange solid.

The crude product was pre-absorbed on silica, loaded on the pre-packed Orochem (40 g) column and eluted at 0-100% of ethyl acetate in pet ether with flow rate 20 ml/min. The pure product was eluted at 45-50% of ethyl acetate in pet ether. The appropriate fractions were collected and concentrated under reduced pressure to afford product 1-bromo-5-iodo-4-methoxy-2-(methylsulfinyl)benzene (1.25 g, 3.12 mmol, 37.3% yield) as orange solid and starting material was recovered (0.4 g)

MS (ESI) calculated for (C₈H₈BrIO₂S) (M+1)⁺, 374.86. found, 374.8.

### Step-4: Synthesis of methyl 4-(5-bromo-2-methoxy-4-(methylsulfinyl)phenyl)-6-methylnicotinate

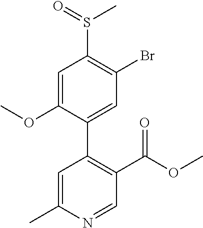

To a stirred solution of 1-bromo-5-iodo-4-methoxy-2-(methylsulfinyl)benzene (600 mg, 1.498 mmol) in 1,4-dioxane (25 mL), was added methyl 4-(5,5-dimethyl-1,3,2-dioxaborinan-2-yl)-6-methylnicotinate (621 mg, 2.246 mmol), potassium carbonate (621 mg, 4.49 mmol) at room temperature. Then the reaction mixture was degassed by nitrogen gas for 10 minutes. PdCl2(dppf) (219 mg, 0.300 mmol) was added. The reaction mixture was stirred at 90° C. for 16 hours. After completion, the reaction mixture was cooled to room temperature, filtered through celite bed and washed with ethyl acetate (40 mL). The filtrate was diluted with water (40 ml), layers were separated and aqueous layer was extracted with ethyl acetate (2×40 mL). The combined organic layer was dried over anhydrous sodium sulfate, filtered and evaporated under reduced pressure to give crude product as a brown liquid.

The crude product was pre-absorbed on silica, loaded on the pre-packed Orochem (50 g) column and eluted at 0-100% of ethyl acetate in pet ether with flow rate 15 ml/min. The pure product was eluted at 75-80% of ethyl acetate in pet ether. The appropriate fractions were collected and concentrated under reduced pressure to afford product methyl 4-(5-bromo-2-methoxy-4-(methylsulfinyl)phenyl)-6-methylnicotinate (330 mg, 0.813 mmol, 54.3% yield) as brown solid.

MS (ESI) calculated for (C₁₆H₁₆BrNO₄S) (M+1)⁺, 398.0. found, 397.8.

### Step-5: Synthesis of 4-(5-bromo-2-methoxy-4-(methylsulfinyl)phenyl)-6-methylnicotinic Acid

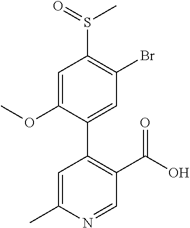 [SMILES: COc1cc(S(C)=O)c(Br)cc1-c1cc(C)ncc1C(=O)O]

To a stirred solution of methyl 4-(5-bromo-2-methoxy-4-(methylsulfinyl)phenyl)-6-methylnicotinate (330 mg, 0.829 mmol) in a mixture tetrahydrofuran (10 mL), and water (2 mL), was added lithium hydroxide monohydrate (174 mg, 4.14 mmol) at room temperature. The reaction mixture was stirred at room temperature for 16 hours. After completion, The reaction mixture was concentrated under reduced pressure. The residue was diluted with water (10 mL) and washed with ethyl acetate (2×15 mL). The aqueous layer was neutralized with 1.5(N) HCl (5 mL) and extracted with ethyl acetate (2×15 mL). The combined organic layer was dried over anhydrous sodium sulfate, filtered and evaporated under reduced pressure to afford 4-(5-bromo-2-methoxy-4-(methylsulfinyl)phenyl)-6-methylnicotinic acid (300 mg, 0.773 mmol, 93% yield) as a brown solid.

MS (ESI) calculated for (C₁₅H₁₄BrNO₄S) (M+1)⁺, 383.99. found 383.8.

### Step-6: Synthesis of 4-(5-bromo-2-methoxy-4-(methylsulfinyl)phenyl)-N-(5-methoxy-1,3,4-thiadiazol-2-yl)-6-methylnicotinamide

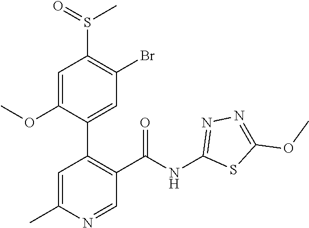

To a stirred solution of 4-(5-bromo-2-methoxy-4-(methylsulfinyl)phenyl)-6-methylnicotinic acid (70 mg, 0.180 mmol) in acetonitrile (5 mL) and N,N-dimethylformamide (0.5 mL) were added 5-methoxy-1,3,4-thiadiazol-2-amine (28.4 mg, 0.216 mmol), 1-methyl-1H-imidazole (0.043 mL, 0.541 mmol) and chloro-N,N,N′,N′-tetramethylformamidinium hexafluorophosphate (76 mg, 0.271 mmol) at room temperature. The reaction mixture was stirred at room temperature for 16 hours. After completion, The reaction mixture was concentrated under reduced pressure. The residue was diluted water (10 ml) and extracted with ethyl acetate (2×10 mL). The combined organic layer was washed with water, brine, dried over sodium sulfate, filtered and concentrated under reduced pressure to give crude product as a brown solid.

The crude product was purified by GRACE revelleris X2 (reverse phase) with the following condition: (Column: 40 g Grace C18 catriage; Mobile Phase A: 0.1% ammonium bicarbonate in water, Mobile Phase B: acetonitrile) 0-100% of B in A for 30 minutes with flow rate 25 mL/min, the pure product was eluted at 40% of B in A. The appropriate fractions were combined and evaporated in vacuum to afford 4-(5-bromo-2-methoxy-4-(methylsulfinyl)phenyl)-N-(5-methoxy-1,3,4-thiadiazol-2-yl)-6-methylnicotinamide (70 mg, 0.134 mmol, 74.4% yield) as white solid.

MS (ESI) calculated for (C₁₈H₁₇BrN₄O₄S₂) (M+2)⁺, 499.0. found, 499.0.

1H-NMR (400 MHz, DMSO-d⁶): δ 12.84 (s, 1H), 8.75 (s, 1H), 7.73 (s, 1H), 7.41 (s, 1H), 7.34 (s, 1H), 4.07 (s, 3H), 3.60 (s, 3H), 2.85 (s, 3H), 2.58 (s, 3H).

### Step 7: Chiral separation of (R)-4-(5-bromo-2-methoxy-4-(methylsulfinyl)phenyl)-N-(5-methoxy-1,3,4-thiadiazol-2-yl)-6-methylnicotinamide and (S)-4-(5-bromo-2-methoxy-4-(methylsulfinyl)phenyl)-N-(5-methoxy-1,3,4-thiadiazol-2-yl)-6-methylnicotinamide

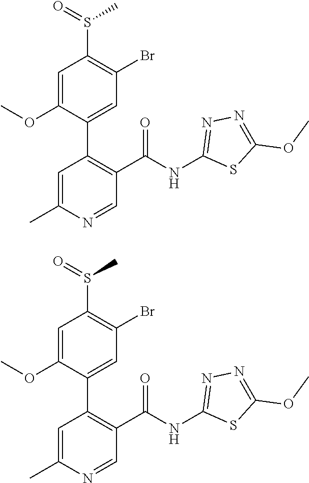

4-(5-Bromo-2-methoxy-4-(methylsulfinyl)phenyl)-N-(5-methoxy-1,3,4-thiadiazol-2-yl)-6-methylnicotinamide (69 mg) was separated by prep-chiral SFC with the following conditions: (Column: YMC Cellulose SZ (250*30) mm, 5 μm; Mobile Phase A: CO₂, Mobile Phase B: Methanol; Gradient: isocratic 40% B; Column Temperature (° C.): 35; Back Pressure (bar): 100; Wave Length: 220 nm; RT1 (min): 4.54; RT2 (min): 5.52; (Sample Solvent: 2 mL THF/MeOH-HPLC; Injection Volume: 0.5 mL/Injection; Cycle time: 7.5 min) to afford (R)-4-(5-bromo-2-methoxy-4-(methylsulfinyl)phenyl)-N-(5-methoxy-1,3,4-thiadiazol-2-yl)-6-methylnicotinamide (30 mg, 0.059 mmol, 44.6% yield) as white solid with the first peak on chiral SFC with shorter retention time and (S)-4-(5-bromo-2-methoxy-4-(methylsulfinyl)phenyl)-N-(5-methoxy-1,3,4-thiadiazol-2-yl)-6-methylnicotinamide (22 mg, 0.043 mmol, 32.6% yield) as white solid with the second peak on chiral SFC with longer retention time.

The absolute stereochemistry was not determined.

### (R)-4-(5-bromo-2-methoxy-4-(methylsulfinyl)phenyl)-N-(5-methoxy-1,3,4-thiadiazol-2-yl)-6-methylnicotinamide

MS (ESI) calculated for (C₁₈H₁₇BrN₄O₄S₂) (M+2)⁺, 499.0. found, 499.0.

1H-NMR (400 MHz, DMSO-d⁶): δ 12.82 (s, 1H), 8.75 (s, 1H), 7.62 (s, 1H), 7.34 (s, 1H), 7.28 (s, 1H), 4.01 (s, 3H), 3.60 (s, 3H), 2.85 (s, 3H), 2.55 (s, 3H).

### (S)-4-(5-bromo-2-methoxy-4-(methylsulfinyl)phenyl)-N-(5-methoxy-1,3,4-thiadiazol-2-yl)-6-methylnicotinamide

MS (ESI) calculated for (C₁₈H₁₇BrN₄O₄S₂) (M+2)⁺, 499.0. found, 499.0.

1H-NMR (400 MHz, DMSO-d⁶): δ 12.85 (s, 1H), 8.80 (s, 1H), 7.66 (s, 1H), 7.34 (s, 1H), 7.30 (s, 1H), 4.03 (s, 3H), 3.60 (s, 3H), 2.85 (s, 3H), 2.56 (s, 3H).

### Example 323

### 6-(azetidin-1-yl)-2′-chloro-5′-methoxy-N-(5-methoxy-1,3,4-thiadiazol-2-yl)-[3,4′-bipyridine]-4-carboxamide

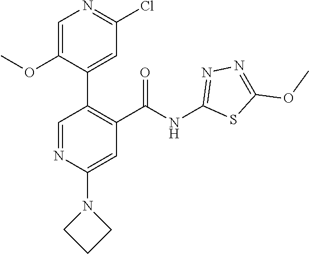

### Step-1: Synthesis of Methyl 2-(azetidin-1-yl)-5-bromoisonicotinate

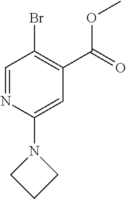

To a stirred solution of methyl 5-bromo-2-chloroisonicotinate (1.5 g, 5.99 mmol) in N,N-dimethylformamide (30 mL) was added sequentially diisopropylethylamine (3.13 mL, 17.97 mmol), followed by azetidine (0.444 mL, 6.59 mmol) at room temperature. The resulting reaction mixture was stirred at 90° C. for 6 h. After completion, the reaction mixture was quenched with water and extracted with ethyl acetate (2×200 mL). The combined organics were washed with brine, dried over sodium sulfate, filtered and concentrated to give crude product as brown solid.

The crude product was pre-absorbed on silica, loaded on the biotage pre-packed column (40 g) and eluted at 0-100% of ethyl acetate in Pet ether for 60 minutes with flow rate 25 ml/min. The pure product was eluted at 10-20% of ethyl acetate in Pet ether. The appropriate fractions were collected and concentrated under reduced pressure to afford methyl 2-(azetidin-1-yl)-5-bromoisonicotinate (700 mg, 2.350 mmol, 39.2% yield) as yellow solid.

MS (ESI) calculated for (C₁₀H₁₁BrN₂O₂) (M+1)⁺, 271.01. found, 271.0.

### Step-2: Synthesis of methyl 6-(azetidin-1-yl)-2′-chloro-5′-methoxy-[3,4′-bipyridine]-4-carboxylate

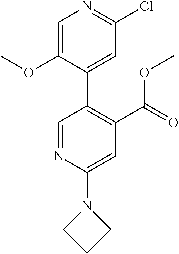 [SMILES: COC(=O)c1cc(N2CCC2)ncc1-c1cc(Cl)ncc1OC]

To a stirred solution of methyl 2-(azetidin-1-yl)-5-bromoisonicotinate (100 mg, 0.369 mmol) in 1,4-dioxane (5 mL) and water (1 mL) were added (2-chloro-5-methoxypyridin-4-yl)boronic acid (83 mg, 0.443 mmol) and potassium carbonate (102 mg, 0.738 mmol) at room temperature and degassed with nitrogen for 15 minutes. After 15 minutes to this was added PdCl2(dppf) (27.0 mg, 0.037 mmol). Then the reaction mixture was stirred at 80° C. for 16 hours. After completion of the reaction, the reaction mixture was cooled to room temperature, inorganic solids were filtered through celite pad. The filtrate was concentrated under reduced pressure to afford crude product as brown gum.

The crude product was pre-absorbed on silica, loaded on the biotage pre-packed column (12 g) and eluted at 0-100% of ethyl acetate in Pet ether for 50 minutes with flow rate 25 ml/min. The pure product was eluated at 5-15% ethyl acetate in Pet ether. The appropriate fractions were collected and concentrated under reduced pressure to afford methyl 6-(azetidin-1-yl)-2′-chloro-5′-methoxy-[3,4′-bipyridine]-4-carboxylate (110 mg, 0.319 mmol, 86% yield) as off-white solid.

MS (ESI) calculated for (C₁₆H₁₆ClN₃O₃) (M+1)⁺, 334.10. found, 334.0.

### Step-3: Synthesis of 6-(azetidin-1-yl)-2′-chloro-5′-methoxy-[3,4′-bipyridine]-4-carboxylic Acid

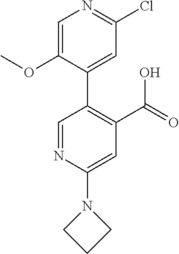 [SMILES: COc1cnc(Cl)cc1-c1cnc(N2CCC2)cc1C(=O)O]

To a stirred solution of methyl 6-(azetidin-1-yl)-2′-chloro-5′-methoxy-[3,4′-bipyridine]-4-carboxylate (110 mg, 0.330 mmol) in tetrahydrofuran (5 mL), methanol (1 mL) and water (1 mL) was added lithiumhydroxide monohydrate (55.3 mg, 1.318 mmol) at 0° C. The reaction mixture was stirred at room temperature for 2 hours. After completion, the reaction mixture was concentrated under reduced pressure. The residue was diluted with water (20 mL), the aqueous was washed with ethyl acetate (25 mL). The aqueous layer was acidified with 1.5N HCl and extracted with 10% methanol in dichloromethane (3×50 mL). The combined organic layer was washed with brine, dried over anhydrous sodium sulfate, filtered and concentrated under reduced pressure to afford 6-(azetidin-1-yl)-2′-chloro-5′-methoxy-[3,4′-bipyridine]-4-carboxylic acid (45 mg, 0.134 mmol, 40.6% yield) as off-white solid

MS (ESI) calculated for (C₁₅H₁₄ClN₃O₃) (M+1)⁺, 320.08. found, 320.0.

### Step-4: Synthesis of 6-(azetidin-1-yl)-2′-chloro-5′-methoxy-N-(5-methoxy-1,3,4-thiadiazol-2-yl)-[3,4′-bipyridine]-4-carboxamide

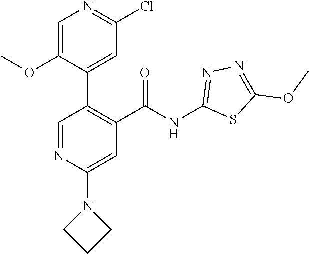

To a stirred solution of 6-(azetidin-1-yl)-2′-chloro-5′-methoxy-[3,4′-bipyridine]-4-carboxylic acid (45 mg, 0.141 mmol), in acetonitrile (3 mL) and N,N-dimethylformamide (1 mL) were added 1-methyl-1H-imidazole (46.2 mg, 0.563 mmol), 5-methoxy-1,3,4-thiadiazol-2-amine (22.15 mg, 0.169 mmol) and chloro-N,N,N′,N′-tetramethylformamidiniumhexafluorophosphate (59.2 mg, 0.211 mmol) at room temperature. The reaction mixture was stirred at room temperature for 4 hours. The reaction mixture was quenched with cold water and concentrated under reduced pressure. The residue was diluted with water (30 mL) and extracted with ethyl acetate (3×30 mL). Combined organic layer was washed with water and brine. The organic layer was dried over anhydrous sodium sulfate, filtered and concentrated under vacuum to afford crude product as orange solid.

The crude product was pre-absorbed on silica, loaded on the biotage pre-packed column (10 g) and eluted at 0-10% of methanol in dichloromethane for 50 minutes with flow rate 25 ml/min. The appropriate fractions were collected and concentrated under reduced pressure to afford 6-(azetidin-1-yl)-2′-chloro-5′-methoxy-N-(5-methoxy-1,3,4-thiadiazol-2-yl)-[3,4′-bipyridine]-4-carboxamide (20.22 mg, 0.046 mmol, 32.8% yield) as off-white solid.

MS (ESI) calculated for (C₁₈H₁₇ClN₆O₃S) (M+1)⁺, 433.09. found, 433.0.

1H-NMR (400 MHz, DMSO-d⁶): δ 12.87 (s, 1H), 8.11 (s, 1H), 8.03 (s, 1H), 7.42 (s, 1H), 6.70 (s, 1H), 4.13-4.03 (m, 7H), 3.57 (s, 3H), 2.44-2.36 (m, 2H).

### Example 325

### N-(5-hydroxy-1,3,4-thiadiazol-2-yl)-5′-methoxy-2′,6-dimethyl-[4,4′-bipyridine]-3-carboxamide

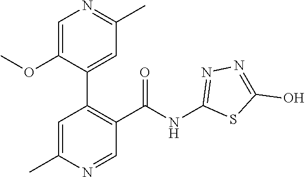 [SMILES: COc1cnc(C)cc1-c1cc(C)ncc1C(=O)Nc1nnc(O)s1]

N-(5-((5-chloropyridin-2-yl)methoxy)-1,3,4-thiadiazol-2-yl)-5′-methoxy-2′,6-dimethyl-[4,4′-bipyridine]-3-carboxamide (5 g, 10.35 mmol, Example 8) was dissolved in 6N HCl (20.71 ml, 124 mmol) and stirred at room temperature for 20 minutes, after which time LCMS (Waters CSH C18 column (30 mm×2.1 mm, 1.7 micron), 0.1% TFA in CH₃CN/H₂O, 2 minutes) indicated that the reaction had only partially progressed. After two hours, LCMS showed further progression of the reaction, but the rate was slow, so 2 mL of concentrated HCl was added and the mixture was stirred overnight, after which time the reaction had progressed further but was not complete. An additional aliquot of concentrated HCl (20.71 ml, 248 mmol) and 12 g of sodium chloride were added and stirred for 30 minutes, after which time cleavage of the benzyl ether was complete. The benzyl chloride by-product, 5-chloro-2-(chloromethyl)pyridine, that formed during the reaction was converted in-situ to the benzylamine, (5-chloropyridin-2-yl)methanamine, because this benzylamine compound is easier to separate from the product than the benzyl chloride. To do this, ammonia, 28-30% (35.0 ml, 518 mmol) was added and stirred for two hours, after which time LCMS indicated that all of the benzyl chloride had been converted to the benzylamine. The pH was adjusted to between 6 and 7, first with concentrated HCl and then with citric acid, which caused the product to precipitate out of solution. The suspension was stirred vigorously and occasionally sonicated until all of the chunks broke up, so that the suspension was a fine white powder in a light yellow liquid. The solid was filtered off and air dried for one hour, then placed under high vacuum overnight to provide N-(5-hydroxy-1,3,4-thiadiazol-2-yl)-5′-methoxy-2′,6-dimethyl-[4,4′-bipyridine]-3-carboxamide (2.55 g, 7.06 mmol, 68% yield). MS (ESI) calc'd for (C₁₆H₁₅N₅O₃S) (M+1)⁺, 358.1. found 358.1. ¹H NMR (400 MHz, DMSO-d₆) δ 12.4 (s, 1H), 8.75 (s, 1H), 8.25 (s, 1H), 7.42 (s, 1H), 7.25 (s, 1H), 3.70 (s, 3H), 2.62 (s, 3H), 2.48 (s, 3H).

### Example 377

### N-(5-methoxy-1,3,4-thiadiazol-2-yl)-4-(4-methoxyphenyl)-6-methylpyridine-3-carboxamide

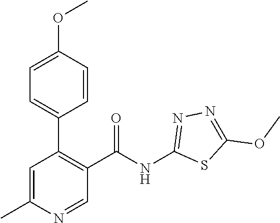

### Step 1: Lithium 4-chloro-6-methylnicotinate

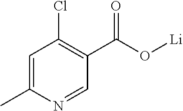

Methyl 4-chloro-6-methylnicotinate (10 g, 53.9 mmol) was suspended in water (20 mL) and 1,4-dioxane (100.0 mL) with rapid stirring and treated with lithium hydroxide (2.261 g, 53.9 mmol) at room temperature. The reaction mixture was stirred at room temperature for 5 minutes, after which time LCMS (Waters CSH C18 column (30 mm×2.1 mm, 1.7 micron), 0.1% TFA in CH₃CN/H₂O, 2 minutes) indicated that the reaction was only partially complete. The reaction mixture was stirred at room temperature for 72 hours, after which time the reaction was complete. The solvent was removed by rotary evaporation, and the crude residue was re-suspended in 200 mL of dioxane and the solvent was again removed by rotary evaporation to azeotrope out any remaining water. This process was repeated twice more, and then the resulting light orange solid was placed under high vacuum for 48 hours to provide lithium 4-chloro-6-methylnicotinate (9.76 g, 53.9 mmol, 100% yield) which was used in the next step as a synthetic intermediate. MS (ESI) calc'd for (C₇H₅ClLiNO₂) (M+H-Li, free acid form from acid/base exchange on LCMS)⁺, 172. found 172.0. ¹H NMR (400 MHz, D₂O) δ 8.45 (s, 1H), 7.38 (s, 1H), 2.50 (s, 3H).

### Step 2: 4-chloro-N-(5-methoxy-1,3,4-thiadiazol-2-yl)-6-methylnicotinamide

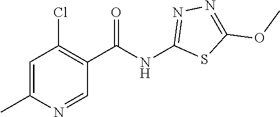

Lithium 4-chloro-6-methylnicotinate (19.57 g, 110 mmol) was suspended in acetonitrile (100 mL) and treated with 5-methoxy-1,3,4-thiadiazol-2-amine (17.35 g, 132 mmol), 1-methylimidazole (30.8 mL, 386 mmol), and N-(chloro(dimethylamino)methylene)-N-methylmethanaminium hexafluorophosphate(V) (46.4 g, 165 mmol) in that order at room temperature under an atmosphere of dry nitrogen. The reaction mixture was stirred at room temperature for 18 hours, after which time LCMS (Waters CSH C18 column (30 mm×2.1 mm, 1.7 micron), 0.1% TFA in CH₃CN/H₂O, 2 minutes) indicated that the reaction was complete. The solvent was removed by rotary evaporation and the resulting residue was poured into a solution of KOH (40 g, 713 mmol) in 2 L of water and stirred vigorously for five minutes in order to hydrolyze the uronium species by-product that was observed by LCMS at a retention time slightly less than that of the desired product. At high pH (˜14), the mixture was a homogeneous solution. A solution of citric acid (128 g, 666 mmol) dissolved in 300 mL of water was then added all at once, causing the product to precipitate. The amount of base to acid in this mixture was calculated to produce a potassium/1-methylimidazolium citrate buffer at a pH of 4. This mixture with a measured pH of 4 was stirred vigorously for 20 minutes. The precipitate was filtered off and washed with water (3×2 L), a 50/50 mixture of acetonitrile/water (1×2 L), acetonitrile (1×500 ml), and diethyl ether (2×1 L). The solid was air dried for one hour and then placed under high vacuum overnight to provide 4-chloro-N-(5-methoxy-1,3,4-thiadiazol-2-yl)-6-methylnicotinamide (25.6 g, 89 mmol, 81% yield) as a pale tan solid which was used in the next step as a synthetic intermediate. MS (ESI) calc'd for (C₁₀H₉ClN₄O₂S) (M+H)+, 285.0. found 285.0. ¹H NMR (400 MHz, DMSO-d₆) δ 13.2 (s, 1H), 8.71 (s, 1H), 7.59 (s, 1H), 4.31 (s, 3H), 2.50 (s, 3H).

### Step 3: N-(5-methoxy-1,3,4-thiadiazol-2-yl)-4-(4-methoxyphenyl)-6-methylnicotinamide

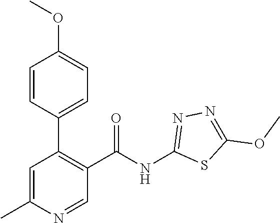

4-Chloro-N-(5-methoxy-1,3,4-thiadiazol-2-yl)-6-methylnicotinamide (0.030 g, 0.105 mmol) was dissolved in dimethyl sulfoxide (DMSO) (2 mL) and treated with (4-methoxyphenyl)boronic acid (0.019 g, 0.126 mmol), potassium carbonate, 2M solution in nitrogen degassed water (0.158 mL, 0.316 mmol), and 1,1′-bis(diphenylphosphino)ferrocene-palladium(II)dichloride dichloromethane complex (8.60 mg, 10.54 μmol) under an atmosphere of dry argon. The reaction mixture was heated to 75° C. and stirred overnight, after which time LCMS (Waters CSH C18 column (30 mm×2.1 mm, 1.7 micron), 0.1% TFA in CH₃CN/H₂O, 2 minutes) indicated that the reaction was complete. The reaction mixture was allowed to cool to room temperature and was then filtered through a 0.2 um PTFE filter disk and purified by reverse phase preparative chromatography (XBridge C18 column (75 mm×30 mm i.d. 5 μm packing diameter) at ambient temperature, Solvent A=10 mM Ammonium Bicarbonate in H₂O adjusted to pH 10 with Ammonia. Solvent B=Acetonitrile. Gradient elution from 95% A/5% B to 0% A/100% B over 12.5 minutes, flow rate=43 mL/min) to provide N-(5-methoxy-1,3,4-thiadiazol-2-yl)-4-(4-methoxyphenyl)-6-methylnicotinamide. MS (ESI) calc'd for (C₁₇H₁₆N₄O₃S) (M+H)⁺, 357.1. found 357.1.

Following general chemistry described above and knowledge known in the art, the following examples were synthesized. Unless otherwise indicated, the stereochemistry of the compounds listed below is not determined and is arbitrarily assigned.

| Example | Structure/Name | ¹H NMR | LCMS |
|---|---|---|---|
| Example 79 | 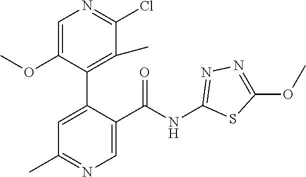 2′-chloro-5′-methoxy-N-(5- methoxy-1,3,4-thiadiazol-2-yl)- 3′,6-dimethyl-(4,4′-bipyridine)-3- carboxamide | ¹H NMR (400 MHz, DMSO-d₆) δ 12.98 (s, 1H), 8.96 (s, 1H), 8.08 (s, 1H), 7.26 (s, 1H), 4.06 (s, 3H), 3.67 (s, 3H), 2.58 (s, 3H), 2.04 (s, 3H). | MS (ESI) calc′d for (C₁₇H₁₆ClN₅O₃S) (M + 1)⁺, 406.1, found 406.1. |
| Example 80 | 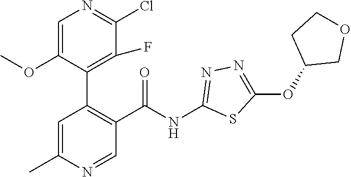 [SMILES: COc1cnc(Cl)c(F)c1-c1cc(C)ncc1C(=O)Nc1nnc(O[C@@H]2CCOC2)s1] 2′-chloro-3′-fluoro-5′-methoxy-6- methyl-N-(5-(((R)- tetrahydrofuran-3-yl)oxy)-1,3,4- thiadiazol-2-yl)-(4,4′-bipyridine)- 3-carboxamide (stereochemistry determined) | ¹H NMR (400 MHz, DMSO-d₆) δ 13.06 (s, 1H), 8.97 (s, 1H), 8.19 (s, 1H), 7.48 (s, 1H), 5.54-5.47 (m, 1H), 3.93-3.81 (m, 3H), 3.81-3.74 (m, 4H), 2.60 (s, 3H), 2.34-2.21 (m, 1H), 2.11-2.01 (m, 1H). | MS (ESI) calc′d for (C₁₉H₁₇ClFN₅O₄S) (M + 1)⁺, 466.1, found, 466.2 |
| Example 81 | 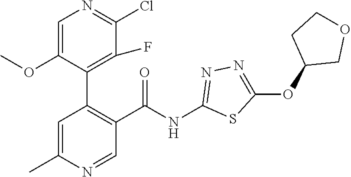 [SMILES: COc1cnc(Cl)c(F)c1-c1cc(C)ncc1C(=O)Nc1nnc(O[C@H]2CCOC2)s1] 2′-chloro-3′-fluoro-5′-methoxy-6- methyl-N-(5-(((S)- tetrahydrofuran-3-yl)oxy)-1,3,4- thiadiazol-2-yl)-(4,4′-bipyridine)- 3-carboxamide (stereochemistry determined) | ¹H NMR (400 MHz, DMSO-d₆) δ 13.07 (s, 1H), 9.01 (s, 1H), 8.17 (s, 1H), 7.42 (s, 1H), 5.49-5.47 (m, 1H), 3.95-3.75 (m, 3H), 3.76-3.72 (m, 4H), 2.59 (s, 3H), 2.31-2.21 (m, 1H), 2.14-2.06 (m, 1H). | MS (ESI) calc′d for (C₁₉H₁₇ClFN₅O₄S) (M + 1)⁺, 466.1, found 466.2. |
| Example 82 | 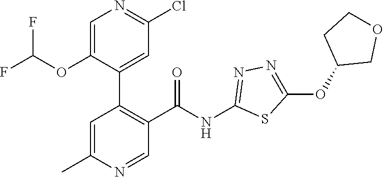 [SMILES: Cc1cc(-c2cc(Cl)ncc2OC(F)F)c(C(=O)Nc2nnc(O[C@@H]3CCOC3)s2)cn1] (R)-2′-chloro-5′- (difluoromethoxy)-6-methyl-N-(5- ((tetrahydrofuran-3-yl)oxy)-1,3,4- thiadiazol-2-yl)-(4,4′-bipyridine)- 3-carboxamide (stereochemistry determined) | ¹H NMR (400 MHz, DMSO-d₆) δ 13.08 (s, 1H), 8 9.08 (s, 1H), 8.27 (s, 1H), 7.52 (s, 1H), 7.20-7.11 (m, 2H), 5.40-5.36 (m, 1H), 3.88-3.78 (m, 3H), 3.78-3.68 (m, 1H), 2.55 (s, 3H), 2.28-2.15 (m, 1H), 2.10-2.00 (m, 1H). | MS (ESI) calc′d for (C₁₉H₁₆ClF₂N₅O₄S) (M + 1)⁺, 484.1. found: 484.2 |
| Example 83 | 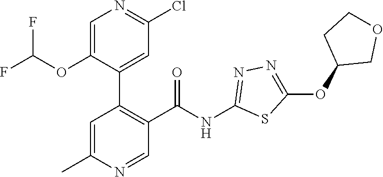 [SMILES: Cc1cc(-c2cc(Cl)ncc2OC(F)F)c(C(=O)Nc2nnc(O[C@H]3CCOC3)s2)cn1] (S)-2′-chloro-5′- (difluoromethoxy)-6-methyl-N-(5- ((tetrahydrofuran-3-yl)oxy)-1,3,4- thiadiazol-2-yl)-(4,4′-bipyridine)- 3-carboxamide (stereochemistry determined) | ¹H NMR (400 MHz, DMSO-d₆) δ 13.07 (s, 1H), 8.98 (s, 1H), 8.32 (s, 1H), 7.69 (s, 1H), 7.43 (s, 1H), 7.32-6.98 (m, 1H), 5.40-5.36 (m, 1H), 3.95-3.80 (m, 3H), 3.79-3.75 (m, 1H), 2.60 (s, 3H), 2.33- 2.20 (m, 1H), 2.16- 2.07 (m, 1H). | MS (ESI) calc′d for (C₁₉H₁₆ClF₂N₅O₄S) (M + 1)⁺, 484.1; found 484.2. |
| Example 84 | 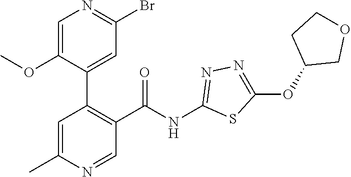 [SMILES: COc1cnc(Br)cc1-c1cc(C)ncc1C(=O)Nc1nnc(O[C@@H]2CCOC2)s1] (R)-N-(1,1-dioxido-2,3- dihydrothiophen-3-yl)-6- (naphthalen-2-yl)-2-oxo-1,2- dihydropyridine-3-carboxamide (stereochemistry determined) | ¹H NMR (400 MHz, DMSO-d₆) δ 12.93 (s, 1H), 8.82 (s, 1H), 8.18 (s, 1H), 7.65 (s, 1H), 7.47 (s, 1H), 5.55-5.48 (m, 1H), 3.96-3.82 (m, 3H), 3.81-3.71 (m, 1H), 3.63 (s, 3H), 2.60 (s, 3H), 2.34-2.22 (m, 1H), 2.18-2.08 (m, 1H). | MS (ESI) calc′d for (C₁₉H₁₈BrN₅O₄S) (M + 1)⁺, 492.0; found 492.1. |
| Example 85 | 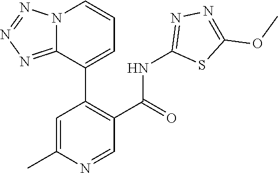 [SMILES: COc1nnc(NC(=O)c2cnc(C)cc2-c2cccn3nnnc23)s1] N-(5-methoxy-1,3,4-thiadiazol-2- yl)-6-methyl-4-(tetrazolo(1,5- a)pyridin-8-yl)nicotinamide | ¹H NMR (400 MHz, DMSO-d₆) δ 13.17 (s, 1H), 9.42-9.36 (m, 1H), 8.97 (s, 1H), 8.00-7.93 (m, 1H), 7.66 (s, 1H), 7.63- 7.55 (m, 1H), 4.05 (s, 3H), 2.66 (s, 3H). | MS (ESI) calc′d for (C₁₅H₁₂N₈O₂S) (M + 1)⁺, 369.1; found, 369.1 |
| Example 88 | 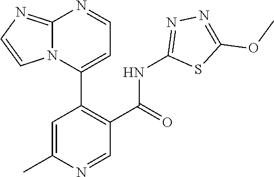 [SMILES: COc1nnc(NC(=O)c2cnc(C)cc2-c2ccnc3nccn23)s1] 4-(imidazo(1,2-a)pyrimidin-5-yl)- N-(5-methoxy-1,3,4-thiadiazol-2- yl)-6-methylnicotinamide | ¹H NMR (400 MHz, DMSO-d₆) δ 13.21 (s, 1H), 9.19 (s, 1H), 8.63 (d, J = 4.4 Hz, 1H), 7.73 (s, 1H), 7.67 (s, 1H), 7.41 (s, 1H), 7.09 (d, J = 4.4 Hz, 1H), 4.04 (s, 3H), 2.66 (s, 3H). | MS (ESI) calc′d for (C₁₆H₁₃N₇O₂S) (M + 1)⁺, 368.1, found 368.1. |
| Example 91 | 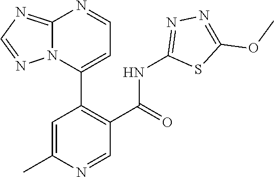 [SMILES: COc1nnc(NC(=O)c2cnc(C)cc2-c2ccnc3ncnn23)s1] 4-((1,2,4)triazolo(1,5-a)pyrimidin- 7-yl)-N-(5-methoxy-1,3,4- thiadiazol-2-yl)-6- methylnicotinamide | ¹H NMR (400 MHz, DMSO-d₆) δ 13.28 (s, 1H), 9.17 (s, 1H), 9.01 (d, J = 4.4 Hz, 1H), 8.53 (s, 1H), 7.64 (s, 1H), 7.53 (d, J = 4.4 Hz, 1H), 4.00 (s, 3H), 2.66 (s, 3H). | MS (ESI) calc′d for (C₁₅H₁₂N₈O₂S) (M + 1)⁺, 369.0, found 369.1 |
| Example 92 | 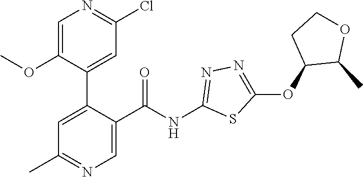 rac-2′-Chloro-5′-methoxy-6- methyl-N-(5-(((2R,3R)-2- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.92 (s, 1H), 8.82 (s, 1H), 8.17 (s, 1H), 7.53 (s, 1H), 7.41 (s, 1H), 5.40 (s, 1H), 3.98- 3.87 (m, 2H), 3.70- 3.34 (m, 4H), 2.58 (s, 3H), 2.47-2.34 (m, 1H), 2.15-2.04 (m, 1H), 1.19 (d, J = 6.4 Hz, 3H). | MS (ESI) calc′d for (C₂₀H₂₀ClN₅O₄S) (M + 1)⁺, 462.1; found, 462.1. |
| Example 93 | 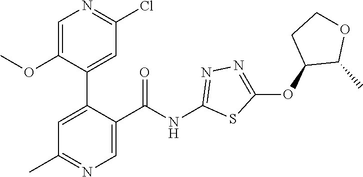 rac-2′-Chloro-5′-methoxy-6- methyl-N-(5-(((2R,3S)-2- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.90 (s, 1H), 8.81 (s, 1H), 8.18 (s, 1H), 7.54 (s, 1H), 7.44 (s, 1H), 5.14-5.07 (m, 1H), 4.17-4.07 (m, 1H), 3.99-3.89 (m, 1H), 3.84-3.74 (m, 1H), 3.64 (s, 3H), 2.60 (s, 3H), 2.39-2.25 (m, 1H), 2.15-2.05 (m, 1H), 1.21 (d, J = 6.4 Hz, 3H). | MS (ESI) calc′d for (C₂₀H₂₀ClN₅O₄S) (M + 1)⁺, 462.1; found, 462.1. |
| Example 94 | 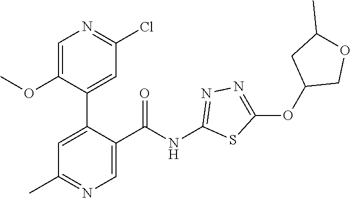 [SMILES: COc1cnc(Cl)cc1-c1cc(C)ncc1C(=O)Nc1nnc(OC2COC(C)C2)s1] 2′-chloro-5′-methoxy-6-methyl-N- (5-((5-methyltetrahydrofuran-3- yl)oxy)-1,3,4-thiadiazol-2-yl)- (4,4′-bipyridine)-3-carboxamide | 1H NMR (400 MHz, DMSO-d₆) δ 12.94 (s, 1H), 8.80 (s, 1H), 8.17 (s, 1H), 7.54 (s, 1H), 7.44 (s, 1H), 5.54-5.42 (m, 1H), 4.22-4.00 (m, 1H), 3.99-3.86 (m, 1H), 3.84-3.73 (m, 1H), 3.64 (s, 3H), 2.59 (s, 3H), 2.63-2.51 (m, 1H), 1.69-1.59 (m, 1H), 1.28-1.17 (m, 3H). | MS (ESI) calc′d for (C₂₀H₂₀ClN₅O₄S) (M + 1)⁺, 462.0, found 462.1. |
| Example 98 | 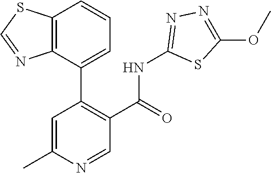 [SMILES: COc1nnc(NC(=O)c2cnc(C)cc2-c2cccc3scnc23)s1] 4-(benzo(d)thiazol-4-yl)-N-(5- methoxy-1,3,4-thiadiazol-2-yl)-6- methylnicotinamide | ¹H NMR (400 MHz, DMSO-d₆) δ 9.36 (s, 1H), 9.07 (d, J = 7.6 Hz, 1H), 8.33 (s, 1H), 8.18 (d, J = 8.0 Hz, 1H), 7.81 (d, J = 7.6 Hz, 1H), 7.62 (d, J = 7.6 Hz, 1H), 7.37 (t, J = 7.6 Hz, 1H), 3.99 (s, 3H), 2.71 (s, 3H). | MS (ESI) calculated for (C₁₇H₁₃N₅O₂S₂) (M + 1)⁺, 384.1, |
| Example 99 | 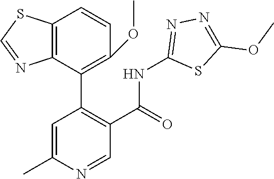 N-(5-methoxy-1,3,4-thiadiazol-2- yl)-4-(5-methoxybenzo(d)thiazol- 4-yl)-6-methylnicotinamide | ¹H NMR (400 MHz, DMSO-d₆) δ 12.84 (s, 1H), 8.84 (s, 1H), 8.16 (s, 1H), 7.57 (s, 1H), 7.42 (s, 1H), 4.08 (s, 3H), 3.63 (s, 3H), 3.43-3.33 (m, 1H), 2.12-2.00 (s, 2H), 1.87-1.75 (m, 4H), 1.70-1.65 (m, 2H). | MS (ESI) calc′d for (C₂₀H₂₀ClN₅O₃S) (M + 1)⁺, 446.1; found, 446.1. |
| Example 100 | 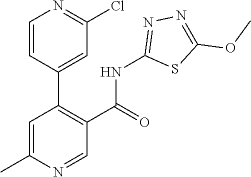 2′-chloro-N-(5-methoxy-1,3,4- thiadiazol-2-yl)-6-methyl-(4,4′- bipyridine)-3-carboxamide | ¹H NMR (400 MHz, DMSO-d₆) δ 13.03 (br, 1H), 8.85 (s, 1H), 8.46 (d, J = 5.2 Hz, 1H), 7.58 (d, J = 1.6 Hz, 1H), 7.51 (s, 1H), 7.36 (dd, J = 5.2, 1.6 Hz, 1H), 4.08 (s, 3H), 2.61 (s, 3H). | MS (ESI) calc′d for (C₁₅H₁₂ClN₅O₂S) (M + 1)⁺, 362.0; found, 362.1. |
| Example 101 | 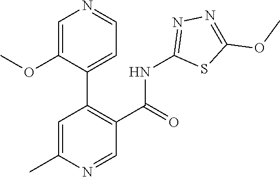 3′-methoxy-N-(5-methoxy-1,3,4- thiadiazol-2-yl)-6-methyl-(4,4′- bipyridine)-3-carboxamide | ¹H NMR (400 MHz, DMSO-d₆) δ 12.85 (b, 1H), 8.80 (s, 1H), 8.38-8.29 (m, 2H), 7.37-7.29 (m, 2H), 4.05 (s, 3H), 3.64 (s, 3H), 2.58 (s, 3H). | (ESI) calc′d for (C₁₆H₁₅N₅O₃S) (M + 1)⁺, 358.1; found 358.1. |
| Example 102 | 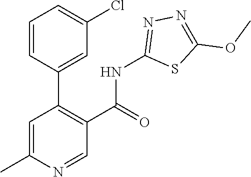 4-(3-chlorophenyl)-N-(5- methoxy-1,3,4-thiadiazol-2-yl)-6- methylnicotinamide | ¹H NMR (400 MHz, DMSO-d₆) δ 12.87 (s, 1H), 8.73 (s, 1H), 7.54-7.42 (m, 4H), 7.30 (d, J = 8.4 Hz, 1H), 4.08 (s, 3H), 2.59 (s, 3H). | MS (ESI) calc′d for (C₁₆H₁₃ClN₄O₂S) (M + 1)⁺, 361.0, 363.0; found, 361.1, 363.1. |
| Example 103 | 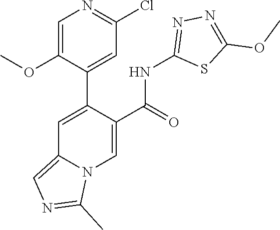 7-(2- chloro-5-methoxypyridin-4-yl)-N- (5-methoxy-1,3,4-thiadiazol-2-yl)- 3-methylimidazo(1,5-a)pyridine- 6-carboxamide | ¹H NMR (400 MHz, DMSO-d₆) δ 12.79 (s, 1H), 8.70 (s, 1H), 8.07 (s, 1H), 7.68 (s, 1H), 7.54 (s, 1H), 7.44 (s, 1H), 4.09 (s, 3H), 3.61 (s, 3H), 2.71 (s, 3H). | MS (ESI) calc′d for (C₁₈H₁₅ClN₆O₃S) (M + 1)⁺, 431.0; found, 431.1. |
| Example 104 | 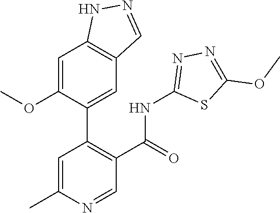 N-(5-methoxy-1,3,4-thiadiazol-2- yl)-4-(6-methoxy-1H-indazol-5- yl)-6-methylnicotinamide | ¹H NMR (400 MHz, DMSO-d₆) δ 12.93 (s, 1H), 12.65 (s, 1H), 8.67 (s, 1H), 8.04 (s, 1H), 7.72 (s, 1H), 7.35 (s, 1H), 6.90 (s, 1H), 4.05 (s, 3H), 3.55 (s, 3H), 2.50 (s, 3H). | MS (ESI) calculated for (C₁₈H₁₆N₆O₃S) (M + 1)⁺, 397.1; found, 397.1. |
| Example 105 | 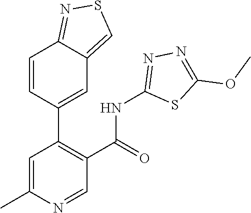 4-(benzo(c)isothiazol-5-yl)-N-(5- methoxy-1,3,4-thiadiazol-2-yl)-6- methylnicotinamide | ¹H NMR (400 MHz, DMSO-d₆) δ 12.92 (s, 1H), 9.88 (d, J = 1.2 Hz, 1H), 8.75 (s, 1H), 7.99 (s, 1H), 7.83 (d, J = 9.2 Hz, 1H), 7.52 (s, 1H), 7.47-7.37 (m, 1H), 4.05 (s, 3H), 2.61 (s, 3H). | MS (ESI) calc′d for (C₁₇H₁₃N₅O₂S₂) (M + 1)⁺, 384.1; found, 384.0. |
| Example 107 | 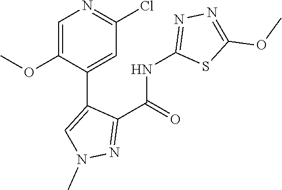 4-(2-chloro-5-methoxypyridin-4- yl)-N-(5-methoxy-1,3,4- thiadiazol-2-yl)-1-methyl-1H- pyrazole-3-carboxamide | ¹H NMR (400 MHz, DMSO-d₆) δ 12.42 (s, 1H), 8.21-8.19 (m, 2H), 7.46 (s, 1H), 4.07 (s, 3H), 3.98 (s, 3H), 3.75 (s, 3H). | MS (ESI) calculated for (C₁₄H₁₃ClN₆O₃S) (M + 1)⁺, 381.0, 383.0; found, 381.1, 383.1. |
| Example 109 | 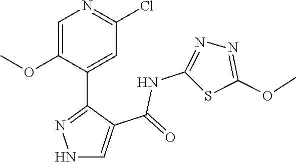 3-(2-chloro-5-methoxypyridin-4- yl)-N-(5-methoxy-1,3,4- thiadiazol-2-yl)-1H-pyrazole-4- carboxamide | 1H-NMR (400 MHz, CD3OD): δ 8.34 (s, 1H), 8.17 (s, 1H), 7.54 (s, 1H), 4.14 (s, 3H), 3.81 (s, 3H). | MS (ESI) calculated for (C₁₃H₁₁ClN₆O₃S) (M + 1)⁺; 367.04; found, 367.0 |
| Example 110 | 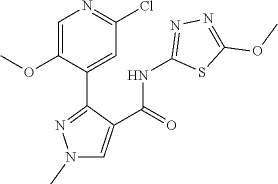 3-(2-chloro-5-methoxypyridin-4- yl)-N-(5-methoxy-1,3,4- thiadiazol-2-yl)-1-methyl-1H- pyrazole-4-carboxamide | ¹H NMR (400 MHz, DMSO-d₆) δ 12.43 (s, 1H), 8.49 (s, 1H), 8.23 (s, 1H), 7.40 (s, 1H), 4.06 (s, 3H), 3.96 (s, 3H), 3.71 (s, 3H). | MS (ESI) calc′d for (C₁₄H₁₃ClN₆O₃S) (M + 1)⁺, 381.0; found, 381.0. |
| Example 112 | 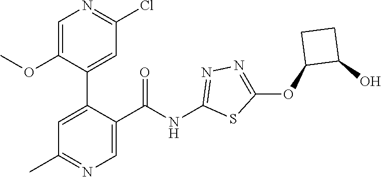 [SMILES: COc1cnc(Cl)cc1-c1cc(C)ncc1C(=O)Nc1nnc(O[C@H]2CC[C@H]2O)s1] rac-2′-chloro-N-(5-((1R,2S)-2- hydroxycyclobutoxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.85 (s, 1H), 8.81 (s, 1H), 8.18 (s, 1H), 7.53 (s, 1H), 7.42 (s, 1H), 5.30-5.29 (m, 1H), 5.12-5.11 (m, 1H), 4.40-4.34 (m, 1H), 3.63 (s, 3H), 2.59 (s, 3H), 2.18-2.07 (m, 3H), 2.07-1.95 (m, 1H) | MS (ESI) calculated for (C₁₉H₁₈ClN₅O₄S) (M + 1)⁺, 448.0; found, 448.1. |
| Example 114 | 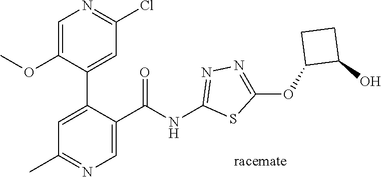 [SMILES: COc1cnc(Cl)cc1-c1cc(C)ncc1C(=O)Nc1nnc(O[C@@H]2CC[C@H]2O)s1] rac-2′-chloro-N-(5-((1R,2R)-2- hydroxycyclobutoxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.89 (s, 1H), 8.81 (s, 1H), 8.17 (s, 1H), 7.53 (s, 1H), 7.43 (s, 1H), 5.60 (d, J = 4.0 Hz, 1H), 4.82-4.79 (m, 1H), 4.18-4.06 (m, 1H), 3.64 (s, 3H), 2.59 (s, 3H), 2.19- 2.16 (m, 1H), 2.06- 2.04 (m, 1H), 1.54- 1.32 (m, 2H) | MS (ESI) calculated for (C₁₉H₁₈ClN₅O₄S) (M + 1)⁺, 448.0; found, 448.1. |
| Example 115 | 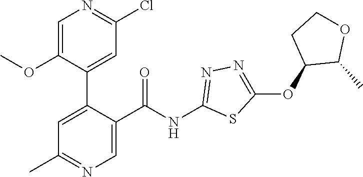 2′-chloro-5′-methoxy-6-methyl-N- (5-(((2R,3S)-2- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.90 (s, 1H), 8.81 (s, 1H), 8.17 (s, 1H), 7.54 (s, 1H), 7.44 (s, 1H), 5.14-5.07 (m, 1H), 4.17-4.07 (m, 1H), 3.99-3.89 (m, 1H), 3.84-3.74 (m, 1H), 3.64 (s, 3H), 2.60 (s, 3H), 2.39-2.25 (m, 1H), 2.15-2.05 (m, 1H), 1.21 (d, J = 6.4 Hz, 3H). | MS (ESI) calc′d for (C₂₀H₂₀ClN₅O₄S) (M + 1)⁺, 462.0; found, 462.1. |
| Example 116 | 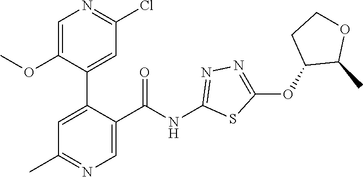 2′-chloro-5′-methoxy-6-methyl-N- (5-(((2S,3R)-2- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.89 (s, 1H), 8.80 (s, 1H), 8.17 (s, 1H), 7.53 (s, 1H), 7.43 (s, 1H), 5.13-5.06 (m, 1H), 4.16-4.07 (m, 1H), 3.98-3.89 (m, 1H), 3.84-3.73 (m, 1H), 3.63 (s, 3H), 2.59 (s, 3H), 2.39-2.25 (m, 1H), 2.15-2.04 (m, 1H), 1.20 (d, J = 6.4 Hz, 3H) | MS (ESI) calc′d for (C₂₀H₂₀ClN₅O₄S) (M + 1)⁺, 462.0; found, 462.1. |
| Example 118 | 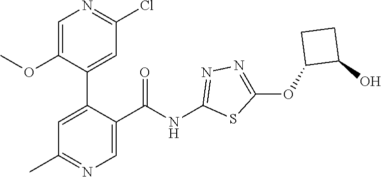 [SMILES: COc1cnc(Cl)cc1-c1cc(C)ncc1C(=O)Nc1nnc(O[C@@H]2CC[C@H]2O)s1] 2′-chloro-N-(5-((1R,2R)-2- hydroxycyclobutoxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.89 (s, 1H), 8.81 (s, 1H), 8.17 (s, 1H), 7.53 (s, 1H), 7.42 (s, 1H), 5.60 (d, J = 6.8 Hz, 1H), 4.85-4.75 (m, 1H), 4.17-4.05 (m, 1H), 3.63 (s, 3H), 2.59 (s, 3H), 2.25- 2.14 (m, 1H), 2.12- 2.00 (m, 1H), 1.54- 1.32 (m, 2H). | MS (ESI) calc′d for (C₁₉H₁₈ClN₅O₄S) (M + 1)⁺, 448.1; found, 448.0. |
| Example 119 | 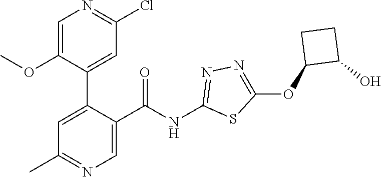 [SMILES: COc1cnc(Cl)cc1-c1cc(C)ncc1C(=O)Nc1nnc(O[C@H]2CC[C@@H]2O)s1] 2′-chloro-N-(5-((1S,2S)-2- hydroxycyclobutoxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.84 (s, 1H), 8.82 (s, 1H), 8.16 (s, 1H), 7.51 (s, 1H), 7.40 (s, 1H), 5.59 (d, J = 6.4 Hz, 1H), 4.84-4.74 (m, 1H), 4.14-4.08 (m, 1H), 3.63 (s, 3H), 2.58 (s, 3H), 2.24- 2.13 (m, 1H), 2.11- 2.00 (m, 1H), 1.54- 1.32 (m, 2H). | MS (ESI) calc′d for (C₁₉H₁₈ClN₅O₄S) (M + 1)⁺, 448.1; found, 448.0. |
| Example 122 | 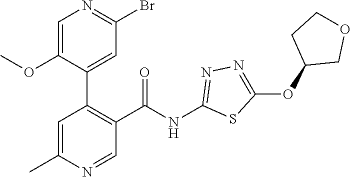 [SMILES: COc1cnc(Br)cc1-c1cc(C)ncc1C(=O)Nc1nnc(O[C@H]2CCOC2)s1] (S)-2′-bromo-5′-methoxy-6- methyl-N-(5-((tetrahydrofuran-3- yl)oxy)-1,3,4-thiadiazol-2-yl)- (4,4′-bipyridine)-3-carboxamide (stereochemistry determined) | ¹H NMR (400 MHz, DMSO-d₆) δ 12.90 (s, 1H), 8.82 (s, 1H), 8.17 (s, 1H), 7.62 (s, 1H), 7.40 (s, 1H), 5.51-5.48 (m, 1H), 3.94-3.83 (m, 3H), 3.79-3.73 (m, 1H), 3.63 (s, 3H), 2.59 (s, 3H), 2.32-2.23 (m, 1H), 2.15-2.09 (m, 1H). | MS (ESI) calculated for (C₁₉H₁₈BrN₅O₄S) (M + 1)⁺, 492.0; found, 492.0. |
| Example 124 | 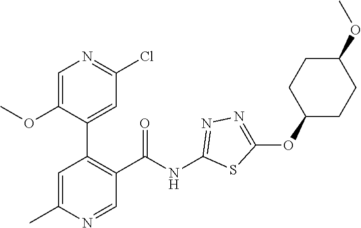 [SMILES: COc1cnc(Cl)cc1-c1cc(C)ncc1C(=O)Nc1nnc(O[C@H]2CC[C@@H](OC)CC2)s1] 2′-chloro-5′-methoxy-N-(5- (((1s,4s)-4- methoxycyclohexyl)oxy)-1,3,4- thiadiazol-2-yl)-6-methyl-(4,4′- bipyridine)-3-carboxamide. The relative stereochemistry was not determined. | ¹H NMR (400 MHz, DMSO-d₆) δ 12.85 (s, 1H), 8.82 (s, 1H), 8.17 (s, 1H), 7.51 (s, 1H), 7.40 (s, 1H), 4.97-4.89 (m, 1H), 3.64 (s, 3H), 3.29- 3.28 (m, 1H), 3.24 (s, 3H), 2.59 (s, 3H), 1.93-1.75 (m, 4H), 1.75-1.61 (m, 4H). | MS (ESI) calc′d for (C₂₂H₂₄ClN₅O₄S) (M + 1)⁺, 490.1; found, 490.2. |
| Example 125 | 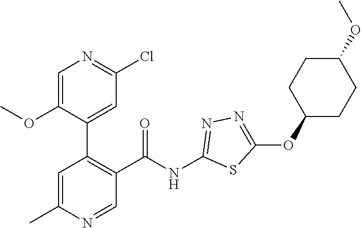 [SMILES: COc1cnc(Cl)cc1-c1cc(C)ncc1C(=O)Nc1nnc(O[C@H]2CC[C@H](OC)CC2)s1] 2′-chloro-5′-methoxy-N-(5- (((1r,4r)-4- methoxycyclohexyl)oxy)-1,3,4- thiadiazol-2-yl)-6-methyl-(4,4′- bipyridine)-3-carboxamide. The relative stereochemistry was not determined. | ¹H NMR (400 MHz, DMSO-d₆) δ 12.75 (s, 1H), 8.84 (s, 1H), 8.16 (s, 1H), 7.49 (s, 1H), 7.37 (s, 1H), 4.92-4.83 (m, 1H), 3.63 (s, 3H), 3.24 (s, 4H), 2.58 (s, 3H), 2.15-2.05 (m, 2H), 1.98-1.89 (m, 2H), 1.64-1.51 (m, 2H), 1.44-1.31 (m, 2H). | MS (ESI) calc′d for (C₂₂H₂₄ClN₅O₄S) (M + 1)⁺, 490.1; found, 490.2. |
| Example 129 | 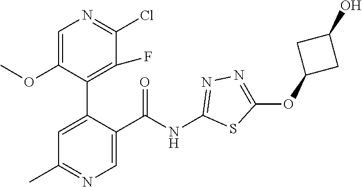 [SMILES: COc1cnc(Cl)c(F)c1-c1cc(C)ncc1C(=O)Nc1nnc(O[C@H]2C[C@@H](O)C2)s1] 2′-chloro-3′-fluoro-N-(5-((1s,3s)- 3-hydroxycyclobutoxy)-1,3,4- thiadiazol-2-yl)-5′-methoxy-6- methyl-(4,4′-bipyridine)-3- carboxamide (stereochemistry determined) | ¹H NMR (400 MHz, DMSO-d₆) δ 13.05 (s, 1H), 8.97 (s, 1H), 8.18 (s, 1H), 7.47 (s, 1H), 5.26-5.21 (m, 1H), 4.77-4.66 (m, 1H), 3.87-3.74 (m, 1H), 3.63 (s, 3H), 2.86-2.76 (m, 2H), 2.60 (s, 3H), 2.06- 1.95 (m, 2H). | MS (ESI) calc′d for (C₁₉H₁₇ClFN₅O₄S) (M + 1)⁺, 466.1; found, 466.1. |
| Example 130 | 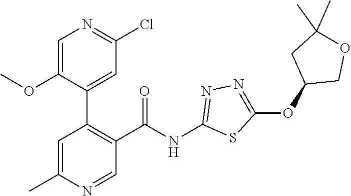 [SMILES: COc1cnc(Cl)cc1-c1cc(C)ncc1C(=O)Nc1nnc(O[C@@H]2COC(C)(C)C2)s1] (S)-2′-chloro-N-(5-((5,5- dimethyltetrahydrofuran-3- yl)oxy)-1,3,4-thiadiazol-2-yl)-5′- methoxy-6-methyl-(4,4′- bipyridine)-3-carboxamide. The stereochemistry was not determined. | ¹H NMR (400 MHz, DMSO-d₆) δ12.90 (s, 1H), 8.81 (s, 1H), 8.17 (s, 1H), 7.54 (s, 1H), 7.43 (s, 1H), 5.53-5.45 (m, 1H), 3.11-4.03 (m, 1H), 3.99-3.92 (m, 1H), 3.64 (s, 3H), 2.59 (s, 3H), 2.05-1.97 (m, 1H), 2.25-2.15 (m, 1H), 1.28 (s, 3H), 1.21 (s, 3H). | MS (ESI) calc′d for (C₂₁H₂₂ClN₅O₄S) (M + 1)⁺, 476.1; found, 476.2 |
| Example 131 | 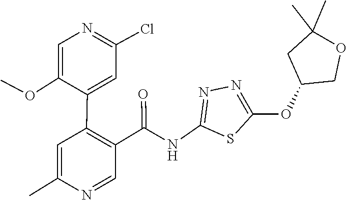 [SMILES: COc1cnc(Cl)cc1-c1cc(C)ncc1C(=O)Nc1nnc(O[C@H]2COC(C)(C)C2)s1] (R)-2′-chloro-N-(5-((5,5- dimethyltetrahydrofuran-3- yl)oxy)-1,3,4-thiadiazol-2-yl)-5′- methoxy-6-methyl-(4,4′- bipyridine)-3-carboxamide. The stereochemistry was not determined. | ¹H NMR (400 MHz, DMSO-d₆) δ 12.90 (s, 1H), 8.81 (s, 1H), 8.17 (s, 1H), 7.54 (s, 1H), 7.43 (s, 1H), 5.53-5.45 (m, 1H), 3.11-4.03 (m, 1H), 3.99-3.92 (m, 1H), 3.64 (s, 3H), 2.59 (s, 3H), 2.05-1.97 (m, 1H), 2.25-2.15 (m, 1H), 1.28 (s, 3H), 1.21 (s, 3H). | MS (ESI) calc′d for (C₂₁H₂₂ClN₅O₄S) (M + 1)⁺, 476.1; found, 476.2. |
| Example 132 | 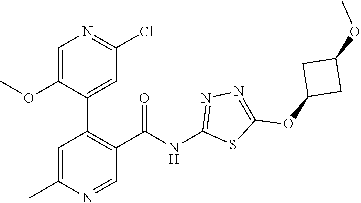 [SMILES: COc1cnc(Cl)cc1-c1cc(C)ncc1C(=O)Nc1nnc(O[C@H]2C[C@@H](OC)C2)s1] 2′-chloro-5′-methoxy-N-(5- ((1s,3s)-3-methoxycyclobutoxy)- 1,3,4-thiadiazol-2-yl)-6-methyl- (4,4′-bipyridine)-3-carboxamide. The relative stereochemistry was not determined. | ¹H NMR (400 MHz, DMSO-d₆) δ 12.90 (s, 1H), 8.82 (s, 1H), 8.17 (s, 1H), 7.51 (s, 1H), 7.40 (s, 1H), 5.29-5.19 (m, 1H), 4.14-4.04 (m, 1H), 3.63 (s, 3H), 3.17 (s, 3H), 2.58 (s, 3H), 2.42-2.38 (m, 4H). | MS (ESI) calc′d for (C₂₀H₂₀ClN₅O₄S) (M + 1)⁺, 462.1; found, 462.1. |
| Example 133 | 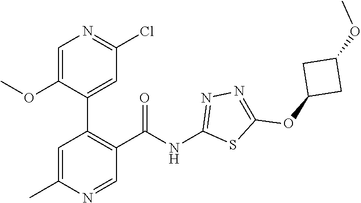 [SMILES: COc1cnc(Cl)cc1-c1cc(C)ncc1C(=O)Nc1nnc(O[C@H]2C[C@H](OC)C2)s1] 2′-chloro-5′-methoxy-N-(5- ((1r,3r)-3-methoxycyclobutoxy)- 1,3,4-thiadiazol-2-yl)-6-methyl- (4,4′-bipyridine)-3-carboxamide. The relative stereochemistry was not determined. | ¹H NMR (400 MHz, DMSO-d₆) δ 12.91 (s, 1H), 8.81 (s, 1H), 8.17 (s, 1H), 7.52 (s, 1H), 7.41 (s, 1H), 4.88-4.78 (m, 1H), 3.63 (s, 4H), 3.16 (s, 3H), 2.91-2.81 (m, 2H), 2.59 (s, 3H), 2.07-1.98 (m, 2H). | MS (ESI) calc′d for (C₂₀H₂₀ClN₅O₄S) (M + 1)⁺, 462.1; found, 462.1. |
| Example 135 | 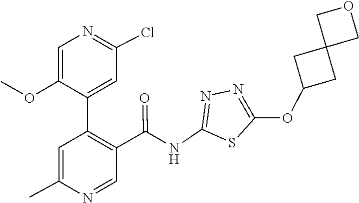 [SMILES: COc1cnc(Cl)cc1-c1cc(C)ncc1C(=O)Nc1nnc(OC2CC3(COC3)C2)s1] N-(5-((2-oxaspiro (3.3) heptan-6- yl)oxy)-1,3,4-thiadiazol-2-yl)-2′- chloro-5′-methoxy-6-methyl-(4,4′- bipyridine)-3-carboxamide | ¹H NMR (400 MHz, DMSO-d₆) δ 12.92 (s, 1H), 8.81 (s, 1H), 8.17 (s, 1H), 7.52 (s, 1H), 7.41 (s, 1H), 5.06-4.96 (m, 1H), 4.63 (s, 2H), 4.55 (s, 2H), 3.63 (s, 3H), 2.82-2.72 (m, 2H), 2.59 (s, 3H), 2.45- 2.31 (m, 2H). | MS (ESI) calc′d for (C₂₁H₂₀ClN₅O₄S) (M + 1)⁺, 474.1; found, 474.1. |
| Example 138 | 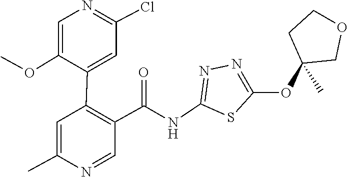 [SMILES: COc1cnc(Cl)cc1-c1cc(C)ncc1C(=O)Nc1nnc(O[C@@]2(C)CCOC2)s1] (S)-2′-chloro-5′-methoxy-6- methyl-N-(5-((3- methyltetrahydrofuran-3-yl)oxy)- 1,3,4-thiadiazol-2-yl)-(4,4′- bipyridine)-3-carboxamide. The absolute stereochemistry was not determined. | ¹H NMR (400 MHz, DMSO-d₆) δ 12.90 (s, 1H), 8.80 (s, 1H), 8.18 (s, 1H), 7.54 (s, 1H), 7.44 (s, 1H), 4.18 (d, J = 9.6 Hz, 1H), 3.92-3.78 (m, 2H), 3.65-3.60 (m, 4H), 2.60 (s, 3H), 2.59-2.52 (m, 1H), 2.15-2.03 (m, 1H), 1.72 (s, 3H). | MS (ESI) calc′d for (C₂₀H₂₀ClN₅O₄S) (M + 1)⁺, 462.0; found, 462.1. |
| Example 139 | 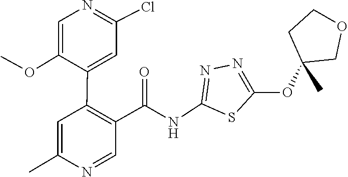 [SMILES: COc1cnc(Cl)cc1-c1cc(C)ncc1C(=O)Nc1nnc(O[C@]2(C)CCOC2)s1] (R)-2′-chloro-5′-methoxy-6- methyl-N-(5-((3- methyltetrahydrofuran-3-yl)oxy)- 1,3,4-thiadiazol-2-yl)-(4,4′- bipyridine)-3-carboxamide. The absolute stereochemistry was not determined. | ¹H NMR (400 MHz, DMSO-d₆) δ 12.90 (s, 1H), 8.80 (s, 1H), 8.18 (s, 1H), 7.54 (s, 1H), 7.43 (s, 1H), 4.19 (d, J = 9.6 Hz, 1H), 3.92-3.78 (m, 2H), 3.69-3.62 (m, 4H), 2.60 (s, 3H), 2.59-2.50 (m, 1H), 2.14-2.02 (m, 1H), 1.72 (s, 3H). | MS (ESI) calc′d for (C₂₀H₂₀ClN₅O₄S) (M + 1)⁺, 462.0; found, 462.1. |
| Example 140 | 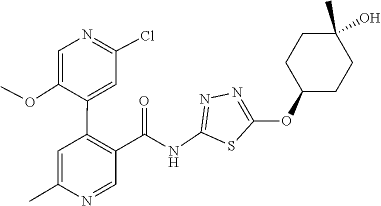 [SMILES: COc1cnc(Cl)cc1-c1cc(C)ncc1C(=O)Nc1nnc(O[C@H]2CC[C@](C)(O)CC2)s1] 2′-chloro-N-(5-(((1r,4r)-4- hydroxy-4- methylcyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.84 (s, 1H), 8.80 (s, 1H), 8.17 (s, 1H), 7.53 (s, 1H), 7.43 (s, 1H), 5.05-4.98 (m, 1H), 4.22 (s, 1H), 3.64 (s, 3H), 2.59 (s, 3H), 2.03-1.91 (m, 2H), 1.84-1.72 (m, 2H), 1.62-1.50 (m, 2H), 1.48-1.38 (m, 2H), 1.15 (s, 3H). | MS (ESI) calculated for (C₂₂H₂₄ClN₅O₄S) (M + 1)⁺, 490.1; found, 490.1. |
| Example 141 | 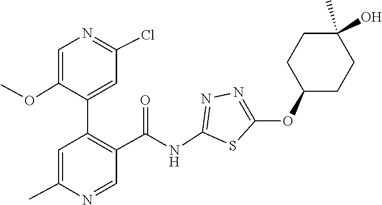 [SMILES: COc1cnc(Cl)cc1-c1cc(C)ncc1C(=O)Nc1nnc(O[C@H]2CC[C@@](C)(O)CC2)s1] 2′-chloro-N-(5-(((1s,4s)-4- hydroxy-4- methylcyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.82 (s, 1H), 8.80 (s, 1H), 8.16 (s, 1H), 7.53 (s, 1H), 7.43 (s, 1H), 4.88-4.76 (m, 1H), 4.21 (s, 1H), 3.64 (s, 3H), 2.59 (s, 3H), 1.95-1.77 (m, 4H), 1.68-1.57 (m, 2H), 1.52-1.36 (m, 2H), 1.12 (s, 3H). | MS (ESI) calculated for (C₂₂H₂₄ClN₅O₄S) (M + 1)⁺, 490.1; found, 490.1. |
| Example 142 | 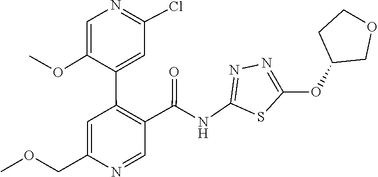 [SMILES: COCc1cc(-c2cc(Cl)ncc2OC)c(C(=O)Nc2nnc(O[C@@H]3CCOC3)s2)cn1] (R)-2′-chloro-5′-methoxy-6- (methoxymethyl)-N-(5- ((tetrahydrofuran-3-yl)oxy)-1,3,4- thiadiazol-2-yl)-(4,4′-bipyridine)- 3-carboxamide (stereochemistry determined) | ¹H NMR (400 MHz, DMSO-d₆) δ 13.00 (s, 1H), 8.89 (s, 1H), 8.19 (s, 1H), 7.54 (s, 1H), 7.52 (s, 1H), 5.59-5.46 (m, 1H), 4.62 (s, 2H), 4.02- 3.81 (m, 3H), 3.81- 3.72 (m, 1H), 3.65 (s, 3H), 3.42 (s, 3H), 2.36-2.22 (m, 1H), 2.19-2.05 (m, 1H). | MS (ESI) calculated for (C₂₀H₂₀ClN₅O₅S) (M + 1)⁺, 478.1; found, 478.0 |
| Example 143 | 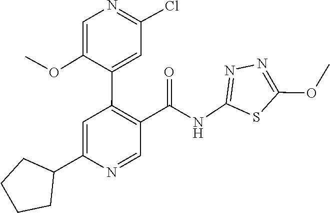 [SMILES: COc1nnc(NC(=O)c2cnc(C3CCCC3)cc2-c2cc(Cl)ncc2OC)s1] 2′-chloro-6-cyclopentyl-5′- methoxy-N-(5-methoxy-1,3,4- thiadiazol-2-yl)-(4,4′-bipyridine)- 3-carboxamide | ¹H NMR (400 MHz, DMSO-d₆) δ 12.84 (s, 1H), 8.84 (s, 1H), 8.16 (s, 1H), 7.57 (s, 1H), 7.42 (s, 1H), 4.08 (s, 3H), 3.63 (s, 3H), 3.43-3.33 (m, 1H), 2.12-2.00 (s, 2H), 1.87-1.75 (m, 4H), 1.70-1.65 (m, 2H). | MS (ESI) calc′d for (C₂₀H₂₀ClN₅O₃S) (M + 1)⁺, 446.1; found, 446.1. |
| Example 145 | 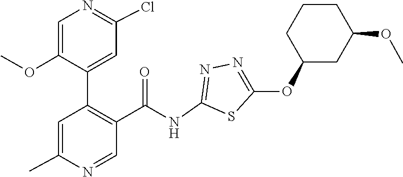 2′-chloro-5′-methoxy-N-(5- (((1S,3R)-3- methoxycyclohexyl)oxy)-1,3,4- thiadiazol-2-yl)-6-methyl-(4,4′- bipyridine)-3-carboxamide. The stereochemistry was not determined. | ¹H NMR (400 MHz, DMSO-d₆) δ 8.79 (s, 1H), 8.16 (s, 1H), 7.52 (s, 1H), 7.41 (s, 1H), 4.86-4.79 (m, 1H), 3.62 (s, 3H), 3.23 (s, 3H), 3.24- 3.22 (m, 1H), 2.58 (s, 3H), 2.56-2.55 (m, 1H), 2.14-2.11 (m, 1H), 1.97-1.94 (m, 1H), 1.79-1.74 (m, 1H), 1.38-1.22 (m, 3H), 1.10-1.01 (m, 1H). | MS (ESI) calculated for (C₂₂H₂₄ClN₅O₄S) (M + 1)⁺, 490.0; found, 490.0. |
| Example 146 | 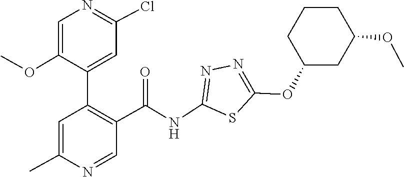 2′-chloro-5′-methoxy-N-(5- (((1R,3S)-3- methoxycyclohexyl)oxy)-1,3,4- thiadiazol-2-yl)-6-methyl-(4,4′- bipyridine)-3-carboxamide. The stereochemistry was not determined. | ¹H NMR (400 MHz, DMSO-d₆) δ 8.79 (s, 1H), 8.16 (s, 1H), 7.53 (s, 1H), 7.42 (s, 1H), 4.88-4.78 (m, 1H), 3.63 (s, 3H), 3.24-3.23 (m, 4H), 2.58 (s, 3H), 2.56- 2.55 (m, 1H), 2.15- 2.13 (m, 1H), 2.01- 1.92 (m, 1H), 1.82- 1.72 (m, 1H), 1.35- 1.250 (m, 3H), 1.11- 1.01 (m, 1H). | MS (ESI) calculated for (C₂₂H₂₄ClN₅O₄S) (M + 1)⁺, 490.0; found, 490.0. |
| Example 147 | 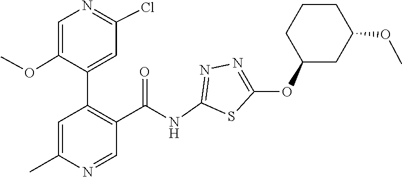 2′-chloro-5′-methoxy-N-(5- (((1S,3S)-3- methoxycyclohexyl)oxy)-1,3,4- thiadiazol-2-yl)-6-methyl-(4,4′- bipyridine)-3-carboxamide. The stereochemistry was not determined. | ¹H NMR (400 MHz, DMSO-d₆) δ 8.82 (s, 1H), 8.16 (s, 1H), 7.51 (s, 1H), 7.40 (s, 1H), 5.16-5.06 (m, 1H), 3.63 (s, 3H), 3.56-3.53 (m, 1H), 3.24 (s, 3H), 2.58 (s, 3H), 2.00-1.98 (m, 1H), 1.92-1.82 (m, 2H), 1.69-1.49 (m, 5H). | MS (ESI) calculated for (C₂₂H₂₄ClF₂N₅O₄S) (M + 1)⁺, 490.0; found, 490.0. |
| Example 148 | 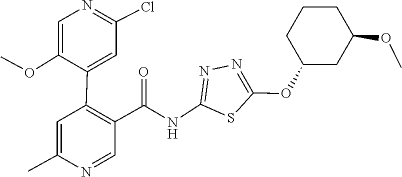 2′-chloro-5′-methoxy-N-(5- (((1R,3R)-3- methoxycyclohexyl)oxy)-1,3,4- thiadiazol-2-yl)-6-methyl-(4,4′- bipyridine)-3-carboxamide. The stereochemistry was not determined. | ¹H NMR (400 MHz, DMSO-d₆) δ 12.05 (s, 1H), 8.81 (s, 1H), 8.17 (s, 1H), 7.53 (s, 1H), 7.42 (s, 1H), 5.16-5.06 (m, 1H), 3.63 (s, 3H), 3.56- 3.55 (m, 1H), 3.24 (s, 3H), 2.58 (s, 3H), 2.00-1.98 (m, 1H), 1.93-1.83 (m, 2H), 1.70-1.50 (m, 5H). | MS (ESI) calculated for (C₂₂H₂₄ClN₅O₄S) (M + 1)⁺, 490.0;found, 490.0. |
| Example 149 | 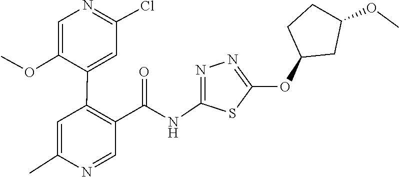 rac-2′-chloro-5′-methoxy-N-(5- (((1R,3R)-3- methoxycyclopentyl)oxy)-1,3,4- thiadiazol-2-yl)-6-methyl-(4,4′- bipyridine)-3-carboxamide. The stereochemistry was not determined. | ^{¹}¹H NMR (400 MHz, DMSO-d₆) δ 12.85 (b, 1H), 8.82 (s, 1H), 8.16 (s, 1H), 7.51 (s, 1H), 7.40 (s, 1H), 5.38-5.28 (m, 1H), 3.99-3.89 (m, 1H), 3.63 (s, 3H), 3.19 (s, 3H), 2.58 (s, 3H), 2.13-2.03 (m, 3H), 1.93-1.75 (m, 2H), 1.75-1.65 (m, 1H). | MS (ESI) calc′d for (C₂₁H₂₂ClN₅O₄S) (M + 1)⁺, 476.1; found, 476.1. |
| Example 150 | 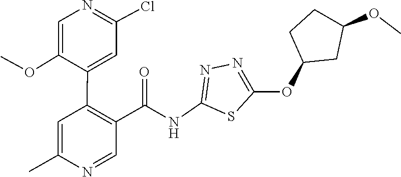 rac-2′-chloro-5′-methoxy-N-(5- (((1R,3S)-3- methoxycyclopentyl)oxy)-1,3,4- thiadiazol-2-yl)-6-methyl-(4,4′- bipyridine)-3-carboxamide. The stereochemistry was not determined. | ^{¹}¹H NMR (400 MHz, DMSO-d₆) δ 12.85 (b, 1H), 8.81 (s, 1H), 8.17 (s, 1H), 7.53 (s, 1H), 7.43 (s, 1H), 5.30-5.20 (m, 1H), 3.87-3.77 (m, 1H), 3.64 (s, 3H), 3.19 (s, 3H), 2.59 (s, 3H), 2.37-2.27 (m, 1H), 2.08-1.98 (m, 1H), 1.97-1.71 (m, 4H). | MS (ESI) calc′d for (C₂₁H₂₂ClN₅O₄S) (M + 1)⁺, 476.1; found, 476.1. |
| Example 151 | 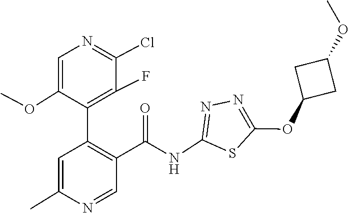 [SMILES: COc1cnc(Cl)c(F)c1-c1cc(C)ncc1C(=O)Nc1nnc(O[C@H]2C[C@H](OC)C2)s1] 2′-chloro-3′-fluoro-5′-methoxy-N- (5-((1r,3r)-3- methoxycyclobutoxy)-1,3,4- thiadiazol-2-yl)-6-methyl-(4,4′- bipyridine)-3-carboxamide. The relative stereochemistry was not determined. | ¹H NMR (400 MHz, DMSO-d₆) δ 13.06 (s, 1H), 8.97 (s, 1H), 8.18 (s, 1H), 7.47 (s, 1H), 4.88-4.78 (m, 1H), 3.74 (s, 3H), 3.67-3.57 (m, 1H), 3.18 (s, 3H), 2.92- 2.80 (m, 2H), 2.60 (s, 3H), 2.09-1.97 (m, 2H). | MS (ESI) calc′d for (C₂₀H₁₉ClFN₅O₄S) (M + 1)⁺, 480.1; found, 480.1 |
| Example 152 | 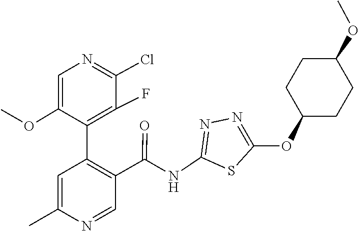 [SMILES: COc1cnc(Cl)c(F)c1-c1cc(C)ncc1C(=O)Nc1nnc(O[C@H]2CC[C@@H](OC)CC2)s1] 2′-chloro-3′-fluoro-5′-methoxy-N- (5-(((1s,4s)-4- methoxycyclohexyl)oxy)-1,3,4- thiadiazol-2-yl)-6-methyl-(4,4′- bipyridine)-3-carboxamide. The relative stereochemistry was not determined. | ¹H NMR (400 MHz, DMSO-d6) δ 13.05 (s, 1H), 8.97 (s, 1H), 8.18 (s, 1H), 7.47 (s, 1H), 4.96-4.86 (m, 1H), 3.74 (s, 3H), 3.30-3.24 (m, 4H), 2.60 (s, 3H), 2.14- 2.02 (m, 2H), 1.94- 1.82 (m, 2H), 1.65- 1.52 (m, 2H), 1.45- 1.31 (m, 2H). | MS (ESI) calc′d for (C₂₂H₂₃ClFN₅O₄S) (M + 1)⁺, 508.1; found, 508.1. |
| Example 153 | 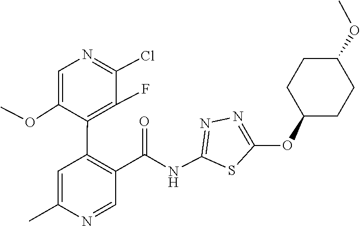 [SMILES: COc1cnc(Cl)c(F)c1-c1cc(C)ncc1C(=O)Nc1nnc(O[C@H]2CC[C@H](OC)CC2)s1] 2′-chloro-3′-fluoro-5′-methoxy-N- (5-(((1r,4r)-4- methoxycyclohexyl)oxy)-1,3,4- thiadiazol-2-yl)-6-methyl-(4,4′- bipyridine)-3-carboxamide. The relative stereochemistry was not determined. | ¹H NMR (400 MHz, DMSO-d6) δ 13.03 (s, 1H), 8.97 (s, 1H), 8.18 (s, 1H), 7.48 (s, 1H), 4.98-4.91 (m, 1H), 3.74 (s, 3H), 3.30-3.16 (m, 4H), 2.60 (s, 3H), 1.88- 1.71 (m, 4H), 1.71- 1.61 (m, 4H). | MS (ESI) calc′d for (C₂₂H₂₃ClFN₅O₄S) (M + 1)⁺, 508.1; found, 508.1. |
| Example 154 | 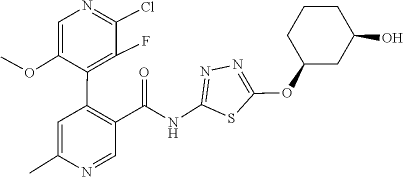 [SMILES: COc1cnc(Cl)c(F)c1-c1cc(C)ncc1C(=O)Nc1nnc(O[C@H]2CCC[C@@H](O)C2)s1] 2′-chloro-3′-fluoro-N-(5- (((1S,3R)-3- hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 13.00 (s, 1H), 8.98 (s, 1H), 8.18 (s, 1H), 7.47 (s, 1H), 5.18 (s, 1H), 4.62 (d, J = 4.0 Hz, 1H), 3.91-3.83 (m, 1H), 3.74 (s, 3H), 2.60 (s, 3H), 1.96- 1.85 (m, 1H), 1.80- 1.77 (m, 2H), 1.70- 1.59 (m, 3H), 1.43- 1.39 (m, 1H), 1.38- 1.35 (m, 1H). | MS (ESI) calc′d for (C₂₁H₂₁ClFN₅O₄S) (M + 1)⁺, 494.1, found 494.1. |
| Example 155 | 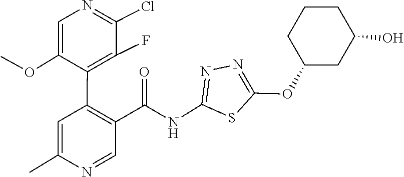 [SMILES: COc1cnc(Cl)c(F)c1-c1cc(C)ncc1C(=O)Nc1nnc(O[C@@H]2CCC[C@H](O)C2)s1] 2′-chloro-3′-fluoro-N-(5- (((1R,3S)-3- hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.97 (s, 1H), 9.00 (s, 1H), 8.17 (s, 1H), 7.42 (s, 1H), 5.16 (s, 1H), 4.61 (d, J = 4.0 Hz, 1H), 3.91-3.83 (m, 1H), 3.74 (s, 3H), 2.59 (s, 3H), 1.95- 1.86 (m, 1H), 1.80- 1.77 (m, 2H), 1.67- 1.59 (m, 3H), 1.43- 1.39 (m, 1H), 1.38- 1.31 (m, 1H). | MS (ESI) calc′d for (C₂₁H₂₁ClFN₅O₄S) (M + 1)⁺, 494.1, found 494.1. |
| Example 156 | 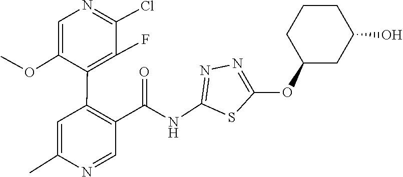 [SMILES: COc1cnc(Cl)c(F)c1-c1cc(C)ncc1C(=O)Nc1nnc(O[C@H]2CCC[C@H](O)C2)s1] 2′-chloro-3′-fluoro-N-(5-(((1S,3S)- 3-hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) ¹H NMR (400 MHz, DMSO- d₆) δ 13.02 (s, 1H), 8.97 (s, 1H), 8.18 (s, 1H), 7.47 (s, 1H), 4.85-4.75 (m, 2H), 3.74 (s, 3H), 3.56- 3.45 (m, 1H), 2.60- 2.57 (s, 3H), 2.40- 2.37 (m, 1H), 2.12- 2.10 (m, 1H), 1.81- 1.78 (m, 1H), 1.72- 1.70 (m, 1H), 1.37- 1.19 (m, 4H). | MS (ESI) calc′d for (C₂₁H₂₁ClFN₅O₄S) (M + 1)⁺, 494.1, found 494.1. |
| Example 157 | 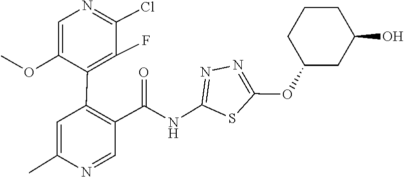 [SMILES: COc1cnc(Cl)c(F)c1-c1cc(C)ncc1C(=O)Nc1nnc(O[C@@H]2CCC[C@@H](O)C2)s1] 2′-chloro-3′-fluoro-N-(5- (((1R,3R)-3- hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 13.02 (s, 1H), 8.98 (s, 1H), 8.18 (s, 1H), 7.46 (s, 1H), 4.86-4.74 (m, 2H), 3.74 (s, 3H), 3.54-3.46 (m, 1H), 2.60 (s, 3H), 2.41- 2.38 (m, 1H), 2.11- 2.08 (m, 1H), 1.80- 1.77 (m, 1H), 1.74- 1.70 (m, 1H), 1.37- 1.19 (m, 3H), 1.16- 1.02 (m, 1H). | MS (ESI) calc′d for (C₂₁H₂₁ClFN₅O₄S) (M + 1)⁺, 494.1, found 494.1. |
| Example 159 | 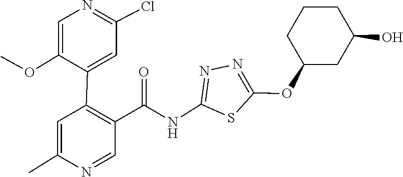 [SMILES: COc1cnc(Cl)cc1-c1cc(C)ncc1C(=O)Nc1nnc(O[C@H]2CCC[C@@H](O)C2)s1] 2′-chloro-N-(5-(((1S,3R)-3- hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.97 (s, 1H), 8.85 (s, 1H), 8.16 (s, 1H), 7.48 (s, 1H), 7.36 (s, 1H), 5.15-5.13 (m, 1H), 4.63-4.58 (m, 1H), 3.87-3.86 (m, 1H), 3.64 (s, 3H), 2.57 (s, 3H), 1.90-1.88 (m, 1H), 1.80-1.78 (m, 2H), 1.64-1.58 (m, 3H), 1.54-1.52 (m, 1H), 1.41-1.34 (m, 1H). | MS (ESI) calc′d for (C₂₁H₂₂ClN₅O₄S) (M + 1)⁺, 476.1, found 476.1. |
| Example 160 | 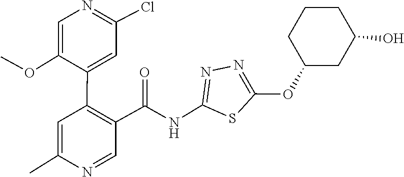 [SMILES: COc1cnc(Cl)cc1-c1cc(C)ncc1C(=O)Nc1nnc(O[C@@H]2CCC[C@H](O)C2)s1] 2′-chloro-N-(5-(((1R,3S)-3- hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 8.90 (s, 1H), 8.28 (s, 1H), 8.16 (s, 1H), 7.42- 7.36 (m, 1H), 7.29- 7.21 (m, 1H), 5.10- 5.08 (m, 1H), 4.58- 4.56 (m, 1H), 3.64 (s, 3H), 2.55 (s, 3H), 1.88-1.84 (m, 1H), 1.77-1.32 (m, 6H). | MS (ESI) calc′d for (C₂₁H₂₂ClN₅O₄S) (M + 1)⁺, 476.1, found 476.1. |
| Example 161 | 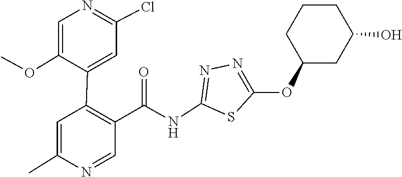 [SMILES: COc1cnc(Cl)cc1-c1cc(C)ncc1C(=O)Nc1nnc(O[C@H]2CCC[C@H](O)C2)s1] 2′-chloro-N-(5-(((1R,3R)-3- hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.86 (s, 1H), 8.83 (s, 1H), 8.17 (s, 1H), 7.50 (s, 1H), 7.39 (s, 1H), 4.81-4.75 (m, 2H), 3.64 (s, 3H), 3.54- 3.46 (m, 1H), 2.58 (s, 3H), 2.44-2.37 (m, 1H), 2.11-2.09 (m, 1H), 1.84-1.76 (m, 1H), 1.37-1.22 (m, 1H), 1.28-1.23 (m, 3H). | MS (ESI) calc′d for (C₂₁H₂₂ClN₅O₄S) (M + 1)⁺, 476.1, found 476.1. |
| Example 162 | 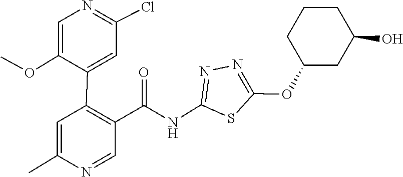 [SMILES: COc1cnc(Cl)cc1-c1cc(C)ncc1C(=O)Nc1nnc(O[C@@H]2CCC[C@@H](O)C2)s1] 2′-chloro-N-(5-(((1S,3S)-3- hydroxycyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 8.85 (s, 1H), 8.15 (s, 1H), 7.47 (s, 1H), 7.35 (s, 1H), 4.76-4.70 (m, 2H), 3.64 (s, 3H), 3.38-3.36 (m, 1H), 2.58 (s, 3H), 2.43- 2.36 (m, 1H), 2.11- 2.10 (m, 1H), 1.84- 1.76 (m, 1H), 1.74- 1.70 (m, 1H), 1.35- 1.22 (m, 3H), 1.12- 1.04 (m, 1H). | MS (ESI) calc′d for (C₂₁H₂₂ClN₅O₄S) (M + 1)⁺, 476.1, found 476.1. |
| Example 163 | 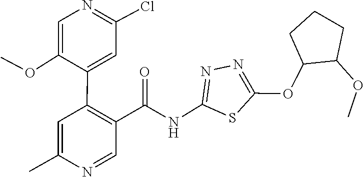 [SMILES: COc1cnc(Cl)cc1-c1cc(C)ncc1C(=O)Nc1nnc(OC2CCCC2OC)s1] 2′-chloro-5′-methoxy-N-(5-((2- methoxycyclopentyl)oxy)-1,3,4- thiadiazol-2-yl)-6-methyl-(4,4′- bipyridine)-3-carboxamide | 1H NMR (400 MHz, DMSO-d6) δ 12.90 (s, 1H), 8.80 (s, 1H), 8.18 (s, 1H), 7.54 (s, 1H), 7.44 (s, 1H), 5.22-5.10 (m, 1H), 3.98-3.79 (m, 1H), 3.64 (s, 3H), 3.31 (s, 3H), 2.59 (s, 3H), 2.16-2.06 (m, 1H), 1.99-1.89 (m, 1H), 1.81-1.56 (m, 4H). | MS (ESI) calculated for (C₂₁H₂₂ClN₅O₄S) (M + 1)⁺, 476.1; found, 476.2. |
| Example 164 | 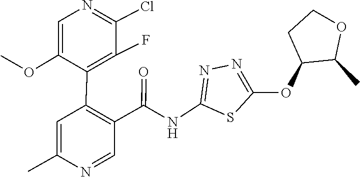 2′-chloro-3′-fluoro-5′-methoxy-6- methyl-N-(5-(((2S,3S)-2- methyltetrahydrofuran-3-yl)oxy)- 1,3,4-thiadiazol-2-yl)-[4,4′- bipyridine]-3-carboxamide. The stereochemistry was not determined. | ¹H NMR (400 MHz, DMSO-d₆) δ 13.06 (s, 1H), 8.97 (s, 1H), 8.19 (s, 1H), 7.48 (s, 1H), 5.39-5.38 (m, 1H), 3.96-3.88 (m, 2H), 3.75 (s, 3H), 3.66-3.62 (m, 1H), 2.62 (s, 3H), 2.44- 2.37 (m, 1H), 2.17- 2.09 (m, 1H), 1.24- 1.20 (m, 3H). | MS (ESI) calc′d for (C₂₀H₁₉ClFN₅O₄S) [M + 1]⁺, 480.0, found 480.1. |
| Exaple 165 | 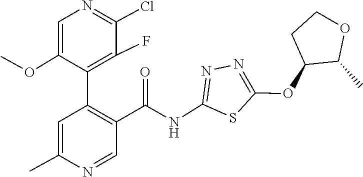 2′-chloro-3′-fluoro-5′-methoxy-6- methyl-N-(5-(((2R,3S)-2- methyltetrahydrofuran-3-yl)oxy)- 1,3,4-thiadiazol-2-yl)-[4,4′- bipyridine]-3-carboxamide. The stereochemistry was not determined. | ¹H NMR (400 MHz, DMSO-d₆) δ 13.04 (s, 1H), 8.97 (s, 1H), 8.19 (s, 1H), 7.49 (s, 1H), 5.10-5.07 (m, 1H), 4.14-4.09 (m, 1H), 3.96-3.91 (m, 1H), 3.21-3.77 (m, 1H), 3.74 (s, 3H), 2.60 (s, 3H), 2.36- 2.26 (m, 1H), 2.10- 2.06 (m, 1H), 1.20 (d, J = 6.4 Hz, 3H). | MS (ESI) calc′d for (C₂₀H₁₉ClFN₅O₄S) [M + 1]⁺, 480.0, found 480.1. |
| Example 166 | 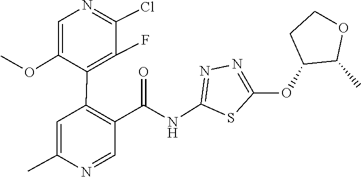 2′-chloro-3′-fluoro-5′-methoxy-6- methyl-N-(5-(((2R,3R)-2- methyltetrahydrofuran-3-yl)oxy)- 1,3,4-thiadiazol-2-yl)-[4,4′- bipyridine]-3-carboxamide. The stereochemistry was not determined. | ¹H NMR (400 MHz, DMSO-d₆) δ 13.06 (s, 1H), 8.97 (s, 1H), 8.19 (s, 1H), 7.48 (s, 1H), 5.39-5.38 (m, 1H), 3.49-3.85 (m, 2H), 3.74 (s, 3H), 3.66-3.62 (m, 1H), 2.63 (s, 3H), 2.47- 2.34 (m, 1H), 2.17- 2.09 (m, 1H), 1.24- 1.20 (m, 3H). | MS (ESI) calc′d for (C₂₀H₁₉ClFN₅O₄S) [M + 1]⁺, 480.0, found 480.1. |
| Example 167 | 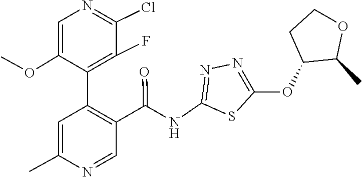 2′-chloro-3′-fluoro-5′-methoxy-6- methyl-N-(5-(((2S,3R)-2- methyltetrahydrofuran-3-yl)oxy)- 1,3,4-thiadiazol-2-yl)-[4,4′- bipyridine]-3-carboxamide. The stereochemistry was not determined. | ¹H NMR (400 MHz, DMSO-d₆) δ 13.06 (s, 1H), 8.97 (s, 1H), 8.15 (s, 1H), 7.47 (s, 1H), 5.10-5.07 (m, 1H), 4.14-4.08 (m, 1H), 3.96-3.91 (m, 1H), 3.81-3.77 (m, 1H), 3.74 (s, 3H), 2.60 (s, 3H), 2.36- 2.26 (m, 1H), 2.10- 2.05 (m, 1H), 1.20 (d, J = 6.4 Hz, 3H). | MS (ESI) calc′d for (C₂₀H₁₉ClFN₅O₄S) [M + 1]⁺, 480.0, found 480.1. |
| Example 168 | 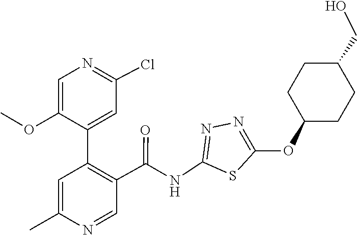 [SMILES: COc1cnc(Cl)cc1-c1cc(C)ncc1C(=O)Nc1nnc(O[C@H]2CC[C@H](CO)CC2)s1] 2′-chloro-N-(5-(((1r,4r)-4- (hydroxymethyl)cyclohexyl)oxy)- 1,3,4-thiadiazol-2-yl)-5′-methoxy- 6-methyl-(4,4′-bipyridine)-3- carboxamide. The relative stereochemistry was determined. | ¹H NMR (400 MHz, DMSO-d₆) δ 12.85 (s, 1H), 8.82 (s, 1H), 8.17 (s, 1H), 7.51 (s, 1H), 7.39 (s, 1H), 4.83-4.71 (m, 1H), 4.48-4.40 (m, 1H), 3.63 (s, 3H), 3.27- 3.20 (m, 2H), 2.58 (s, 3H), 2.25-2.16 (m, 2H), 1.86-1.77 (m, 2H), 1.50-1.34 (m, 3H), 1.12-0.97 (m, 2H). | MS (ESI) calc′d for (C₂₂H₂₄ClN₅O₄S) (M + 1)⁺, 490.1; found, 490.1. |
| Examplse 169 | 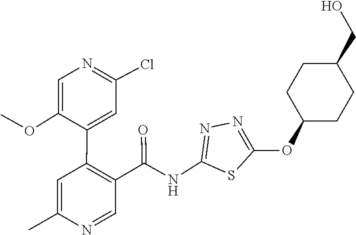 [SMILES: COc1cnc(Cl)cc1-c1cc(C)ncc1C(=O)Nc1nnc(O[C@H]2CC[C@@H](CO)CC2)s1] 2′-chloro-N-(5-(((1s,4s)-4- (hydroxymethyl)cyclohexyl)oxy)- 1,3,4-thiadiazol-2-yl)-5′-methoxy- 6-methyl-(4,4′-bipyridine)-3- carboxamide. The relative stereochemistry was determined. | ¹H NMR (400 MHz, DMSO-d₆) δ 12.83 (s, 1H), 8.80 (s, 1H), 8.18 (s, 1H), 7.53 (s, 1H), 7.43 (s, 1H), 5.09 (s, 1H), 4.41 (t, J = 5.2 Hz, 1H), 3.64 (s, 3H), 3.31-3.23 (m, 2H), 2.59 (s, 3H), 2.08-1.98 (m, 2H), 1.69-1.52 (m, 5H), 1.34-1.20 (m, 2H). | MS (ESI) calc′d for (C₂₂H₂₄ClN₅O₄S) (M + 1)⁺, 490.1, found 490.1. |
| Example 170 | 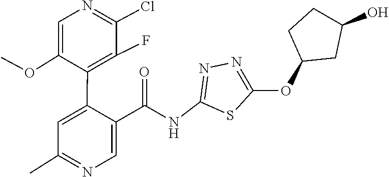 [SMILES: COc1cnc(Cl)c(F)c1-c1cc(C)ncc1C(=O)Nc1nnc(O[C@H]2CC[C@@H](O)C2)s1] rac-2′-chloro-3′-fluoro-N-(5- (((1R,3S)-3- hydroxycyclopentyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 13.01 (s, 1H), 8.97 (s, 1H), 8.18 (s, 1H), 7.48 (s, 1H), 5.21-5.19 (m, 1H), 4.68-4.65 (m, 1H), 4.13-4.08 (m, 1H), 3.74 (s, 3H), 2.60 (s, 3H), 2.32- 2.29 (m, 1H), 2.06- 1.88 (m, 2H), 1.81- 1.59 (m, 3H). | MS (ESI) calculated for (C₂₀H₁₉ClFN₅O₄S) (M + 1)⁺, 480.1; found, 480.2. |
| Example 171 | 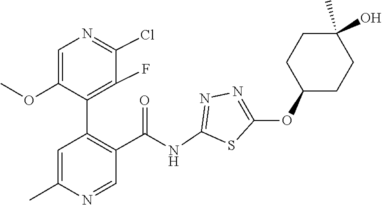 [SMILES: COc1cnc(Cl)c(F)c1-c1cc(C)ncc1C(=O)Nc1nnc(O[C@H]2CC[C@@](C)(O)CC2)s1] 2′-chloro-3′-fluoro-N-(5-(((1s,4s)- 4-hydroxy-4- methylcyclohexyl)oxy)-1,3,4- thiadiazol-2-yl)-5′-methoxy-6- methyl-(4,4′-bipyridine)-3- carboxamide. The relative stereochemistry was not determined. | ¹H NMR (400 MHz, DMSO-d₆) δ 13.00 (s, 1H), 8.98 (s, 1H), 8.18 (s, 1H), 7.45 (s, 1H), 4.85-4.76 (m, 1H), 4.20 (s, 1H), 3.74 (s, 3H), 2.59 (s, 3H), 1.90-1.76 (m, 4H), 1.61-1.48 (m, 2H), 1.47-1.36 (m, 2H), 1.12 (s, 3H). | MS (ESI) calc′d for (C₂₂H₂₃ClFN₅O₄S) (M + 1)⁺, 508.1; found, 508.1. |
| Example 172 | 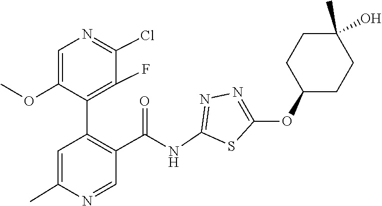 [SMILES: COc1cnc(Cl)c(F)c1-c1cc(C)ncc1C(=O)Nc1nnc(O[C@H]2CC[C@](C)(O)CC2)s1] 2′-chloro-3′-fluoro-N-(5-(((1r,4r)- 4-hydroxy-4- methylcyclohexyl)oxy)-1,3,4- thiadiazol-2-yl)-5′-methoxy-6- methyl-(4,4′-bipyridine)-3- carboxamide. The relative stereochemistry was not determined. | ¹H NMR (400 MHz, DMSO-d₆) δ 12.97 (s, 1H), 8.97 (s, 1H), 8.18 (s, 1H), 7.47 (s, 1H), 5.00 (s, 1H), 4.21 (s, 1H), 3.74 (s, 3H), 2.60 (s, 3H), 2.02-1.91 (m, 2H), 1.82-1.72 (m, 2H), 1.61-1.50 (m, 2H), 1.48-1.37 (m, 2H), 1.15 (s, 3H). | MS (ESI) calc′d for (C₂₂H₂₃ClFN₅O₄S) (M + 1)⁺, 508.1; found, 508.1. |
| Example 175 | 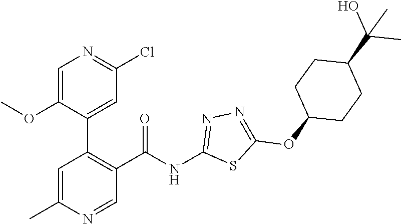 [SMILES: COc1cnc(Cl)cc1-c1cc(C)ncc1C(=O)Nc1nnc(O[C@H]2CC[C@@H](C(C)(C)O)CC2)s1] 2′-chloro-N-(5-(((1s,4s)-4-(2- hydroxypropan-2- yl)cyclohexyl)oxy)-1,3,4- thiadiazol-2-yl)-5′-methoxy-6- methyl-(4,4′-bipyridine)-3- carboxamide. The relative stereochemistry was determined. | ¹H NMR (400 MHz, DMSO-d₆) δ 12.87 (s, 1H), 8.84 (s, 1H), 8.16 (s, 1H), 7.49 (s, 1H), 7.37 (s, 1H), 5.08-5.02 (m, 1H), 4.05 (s, 1H), 3.64 (s, 3H), 2.58 (s, 3H), 2.17-2.09 (m, 2H), 1.67-1.59 (m, 2H), 1.59-1.49 (m, 2H), 1.28 (s, 3H), 1.04 (s, 6H) | MS (ESI) calc′d for (C₂₄H₂₈ClN₅O₄S) (M + 1)⁺, 518.2, found 518.2. |
| Example 176 | 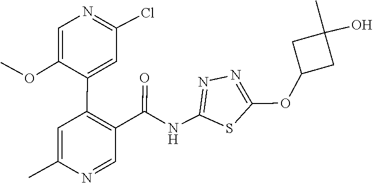 [SMILES: COc1cnc(Cl)cc1-c1cc(C)ncc1C(=O)Nc1nnc(OC2CC(C)(O)C2)s1] 2′-chloro-N-(5-(3-hydroxy-3- methylcyclobutoxy)-1,3,4- thiadiazol-2-yl)-5′-methoxy-6- methyl-(4,4′-bipyridine)-3- carboxamide | Product is a mixture of diastereomers that were not further separated (ratio = 4:1). ¹H NMR (major isomer) (400 MHz, DMSO-d₆) δ 12.89 (s, 1H), 8.80 (s, 1H), 8.17 (s, 1H), 7.54 (s, 1H), 7.43 (s, 1H), 5.22 (s, 1H), 4.90- 4.78 (m, 1H), 3.63 (s, 3H), 2.59 (s, 3H), 2.58-2.51 (m, 2H), 2.26-2.15 (m, 2H), 1.25 (s, 3H). The activity data for this Example is the potency for the mixture. | MS (major isomer) (ESI) calc′d for (C₂₀H₂₀ClN₅O₄S) (M + 1)⁺, 462.0, found, 462.1. |
| Example 177 | 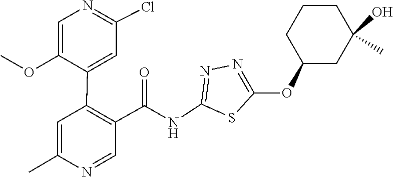 rac-2′-chloro-N-(5-(((1R,3S)-3- hydroxy-3- methylcyclohexyl)oxy)-1,3,4- thiadiazol-2-yl)-5′-methoxy-6- methyl-(4,4′-bipyridine)-3- carboxamide. The relative stereochemistry was not determined. | ¹H NMR (400 MHz, DMSO-d₆) δ 12.86 (s, 1H), 8.81 (s, 1H), 8.17 (s, 1H), 7.53 (s, 1H), 7.42 (s, 1H), 4.92-4.82 (m, 1H), 4.55 (s, 1H), 3.64 (s, 3H), 2.59 (s, 3H), 2.15-2.04 (m, 2H), 1.72-1.48 (m, 4H), 1.44-1. 32 (m, 2H), 1.14 (s, 3H). | MS (ESI) calc′d for (C₂₂H₂₄ClN₅O₄S) (M + 1)⁺, 490.1, found 490.1. |
| Example 178 | 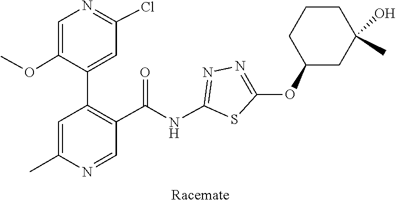 rac-2′-chloro-N-(5-(((1R,3R)-3- hydroxy-3- methylcyclohexyl)oxy)-1,3,4- thiadiazol-2-yl)-5′-methoxy-6- methyl-(4,4′-bipyridine)-3- carboxamide. The relative stereochemistry was not determined. | ¹H NMR (400 MHz, DMSO-d₆) δ 12.86 (s, 1H), 8.81 (s, 1H), 8.17 (s, 1H), 7.53 (s, 1H), 7.42 (s, 1H), 5.10-5.07 (m, 1H), 4.32 (s, 1H), 3.64 (s, 3H), 2.59 (s, 3H), 2.15-2.04 (m, 2H), 1.66-1.20 (m, 6H), 1.14 (s, 3H). | MS (ESI) calc′d for (C₂₂H₂₄ClN₅O₄S) (M + 1)⁺, 490.1, found 490.1. |
| Example 179 | 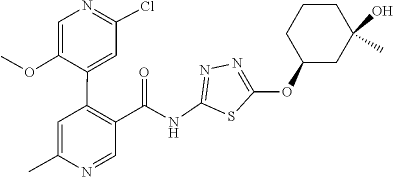 2′-chloro-N-(5-(((1S,3R)-3- hydroxy-3- methylcyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.86 (s, 1H), 8.81 (s, 1H), 8.17 (s, 1H), 7.53 (s, 1H), 7.42 (s, 1H), 4.92-4.82 (m, 1H), 4.55 (s, 1H), 3.64 (s, 3H), 2.59 (s, 3H), 2.15-2.04 (m, 2H), 1.75-1.71 (m, 1H), 1.60-1.48 (m, 2H), 1.44-1.32 (m, 3H), 1.14 (s, 3H). | MS (ESI) calc′d for (C₂₂H₂₄ClN₅O₄S) (M + 1)⁺, 490.1; found, 490.1. |
| Example 180 | 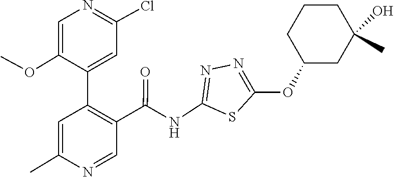 2′-chloro-N-(5-(((1R,3S)-3- hydroxy-3- methylcyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ12.86 (s, 1H), 8.81 (s, 1H), 8.17 (s, 1H), 7.53 (s, 1H), 7.42 (s, 1H), 4.92-4.82 (m, 1H), 4.55 (s, 1H), 3.64 (s, 3H), 2.59 (s, 3H), 2.15-2.04 (m, 2H), 1.75-1.71 (m, 1H), 1.60-1.48 (m, 2H), 1.44-1.32 (m, 3H), 1.14 (s, 3H). | MS (ESI) calc′d for (C₂₂H₂₄ClN₅O₄S) (M + 1)⁺, 490.1; found, 490.1. |
| Example 181 | 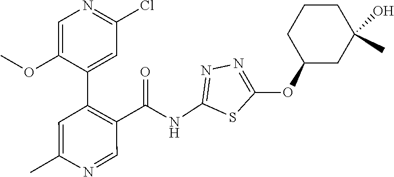 2′-chloro-N-(5-(((1S,3S)-3- hydroxy-3- methylcyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.84 (s, 1H), 8.81 (s, 1H), 8.17 (s, 1H), 7.52 (s, 1H), 7.41 (s, 1H), 5.14-5.02 (m, 1H), 4.32 (s, 1H), 3.64 (s, 3H), 2.59 (s, 3H), 2.20-2.05 (m, 2H), 1.77-1.20 (m, 6H), 1.16 (s, 3H). | MS (ESI) calc′d for (C₂₂H₂₄ClN₅O₄S) (M + 1)⁺, 490.1; found, 490.1. |
| Example 182 | 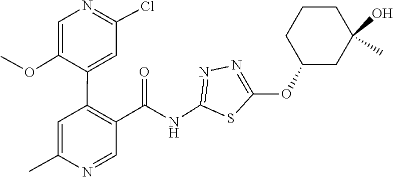 2′-chloro-N-(5-(((1R,3R)-3- hydroxy-3- methylcyclohexyl)oxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ12.84 (s, 1H), 8.81 (s, 1H), 8.17 (s, 1H), 7.52 (s, 1H), 7.41 (s, 1H), 5.14-5.02 (m, 1H), 4.32 (s, 1H), 3.64 (s, 3H), 2.59 (s, 3H), 2.20-2.05 (m, 2H), 1.77-1.20 (m, 6H), 1.16 (s, 3H). | MS (ESI) calc′d for (C₂₂H₂₄ClN₅O₄S) (M + 1)⁺, 490.1; found, 490.1. |
| Example 183 | 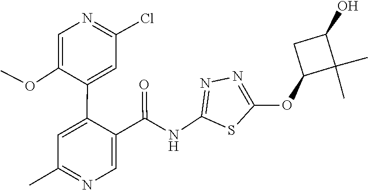 2′-chloro-N-(5-((1r,3s)-3-hydroxy- 2,2-dimethylcyclobutoxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.85 (s, 1H), 8.80 (s, 1H), 8.18 (s, 1H), 7.54 (s, 1H), 7.43 (s, 1H), 5.03 (d, J = 6.0 Hz, 1H), 4.55 (t, J = 7.6 Hz, 1H), 3.63 (s, 3H), 3.56-3.48 (m, 1H), 2.75-2.63 (m, 1H), 2.59 (s, 3H), 2.02- 1.90 (m, 1H), 1.18 (s, 3H), 0.95 (s, 3H). | MS (ESI) calc′d for (C₂₁H₂₂ClN₅O₄S) (M + 1)⁺, 476.1; found, 476.2. |
| Example 184 | 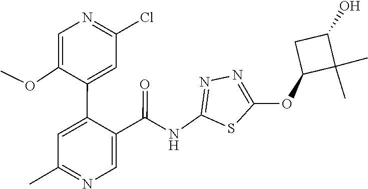 2′-chloro-N-(5-((1r,3r)-3-hydroxy- 2,2-dimethylcyclobutoxy)-1,3,4- thiadiazol-2-yl)-5′-methoxy-6- methyl-(4,4′-bipyridine)-3- carboxamide. The stereochemistry was not determined. | ¹H NMR (400 MHz, DMSO-d₆) δ 12.85 (s, 1H), 8.80 (s, 1H), 8.18 (s, 1H), 7.54 (s, 1H), 7.43 (s, 1H), 5.04 (d, J = 5.6 Hz, 1H), 4.55 (t, J = 7.6 Hz, 1H), 3.63 (s, 3H), 3.55-3.49 (m, 1H), 2.75-2.63 (m, 1H), 2.59 (s, 3H), 2.02- 1.90 (m, 1H), 1.18 (s, 3H), 0.95 (s, 3H | MS (ESI) calc′d for (C₂₁H₂₂ClN₅O₄S) (M + 1)⁺, 476.1; found, 476.2. |
| Example 187 | 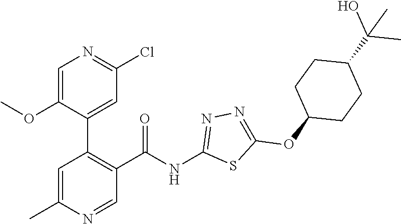 [SMILES: COc1cnc(Cl)cc1-c1cc(C)ncc1C(=O)Nc1nnc(O[C@H]2CC[C@H](C(C)(C)O)CC2)s1] 2′-chloro-N-(5-(((1r,4r)-4-(2- hydroxypropan-2- yl)cyclohexyl)oxy)-1,3,4- thiadiazol-2-yl)-5′-methoxy-6- methyl-(4,4′-bipyridine)-3- carboxamide. The relative stereochemistry was determined. | ¹H NMR (400 MHz, DMSO-d₆) δ 12.86 (s, 1H), 8.80 (s, 1H), 8.17 (s, 1H), 7.53 (s, 1H), 7.43 (s, 1H), 4.82-4.71 (m, 1H), 4.10 (s, 1H), 3.63 (s, 3H), 2.59 (s, 3H), 2.23-2.21 (m, 2H), 1.87-1.85 (m, 2H), 1.47-1.34 (m, 2H), 1.27-1.08 (m, 3H), 1.05 (s, 6H). | MS (ESI) calc′d for (C₂₄H₂₈ClN₅O₄S) (M + 1)⁺, 518.1; found, 518.2 |
| Example 188 | 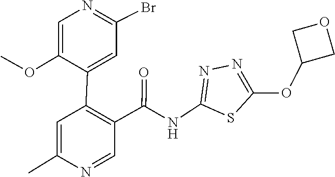 [SMILES: COc1cnc(Br)cc1-c1cc(C)ncc1C(=O)Nc1nnc(OC2COC2)s1] 2′- bromo-5′-methoxy-6-methyl-N-(5- (oxetan-3-yloxy)-1,3,4-thiadiazol- 2-yl)-(4,4′-bipyridine)-3- carboxamide | ¹H NMR (400 MHz, DMSO-d₆) δ 12.99 (s, 1H), 8.80 (s, 1H), 8.18 (s, 1H), 7.64 (s, 1H), 7.43 (s, 1H), 5.72-5.62 (m, 1H), 4.94-4.86 (m, 2H), 4.71-4.61 (m, 2H), 3.62 (s, 3H), 2.59 (s, 3H). | MS (ESI) calc′d for (C₁₈H₁₆BrN₅O₄S) (M + 1)⁺, 478.0; found, 478.0. |
| Example 189 | 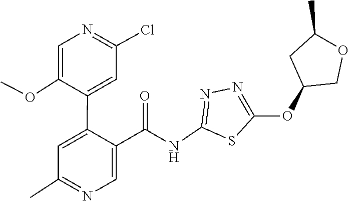 [SMILES: COc1cnc(Cl)cc1-c1cc(C)ncc1C(=O)Nc1nnc(O[C@@H]2CO[C@H](C)C2)s1] 2′-chloro-5′-methoxy-6-methyl-N- (5-(((3S,5R)-5- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.90 (s, 1H), 8.81 (s, 1H), 8.17 (s, 1H), 7.53 (s, 1H), 7.42 (s, 1H), 5.50-5.42 (m, 1H), 4.03 (d, J = 12.0 Hz, 1H), 3.99-3.86 (m, 1H), 3.81-3.73 (m, 1H), 3.63 (s, 3H), 2.61-2.57 (m, 3H), 2.62-2.53 (m, 1H), 1.69-1.58 (m, 1H), 1.25 (d, J = 6.0 Hz, 3H). | MS (ESI) calc′d for (C₂₀H₂₀ClN₅O₄S) (M + 1)⁺, 462.0, found 462.1. |
| Example 190 | 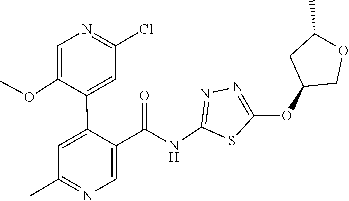 [SMILES: COc1cnc(Cl)cc1-c1cc(C)ncc1C(=O)Nc1nnc(O[C@@H]2CO[C@@H](C)C2)s1] 2′-chloro-5′-methoxy-6-methyl-N- (5-(((3S,5S)-5- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.87 (s, 1H), 8.81 (s, 1H), 8.17 (s, 1H), 7.53 (s, 1H), 7.42 (s, 1H), 5.53-5.46 (m, 1H), 4.22-4.05 (m, 2H), 3.84-3.76 (m, 1H), 3.63 (s, 3H), 2.59 (s, 3H), 2.33-2.23 (m, 1H), 1.83-1.71 (m, 1H), 1.20 (d, J = 6.0 Hz, 3H). | MS (ESI) calc′d for (C₂₀H₂₀ClN₅O₄S) (M + 1)⁺, 462.0, found 462.1 |
| Example 191 | 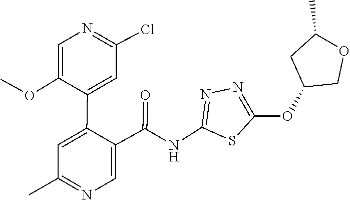 [SMILES: COc1cnc(Cl)cc1-c1cc(C)ncc1C(=O)Nc1nnc(O[C@H]2CO[C@@H](C)C2)s1] 2′-chloro-5′-methoxy-6-methyl-N- (5-(((3R,5S)-5- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.90 (s, 1H), 8.81 (s, 1H), 8.17 (s, 1H), 7.54 (s, 1H), 7.43 (s, 1H), 5.50-5.42 (m, 1H), 4.04 (d, J = 12.0 Hz, 1H), 3.99-3.86 (m, 1H), 3.81-3.73 (m, 1H), 3.64 (s, 3H), 2.59 (s, 3H), 2.63- 2.51 (m, 1H), 1.69- 1.59 (m, 1H), 1.25 (d, J = 6.4 Hz, 3H). | MS (ESI) calc′d for (C₂₀H₂₉ClN₅O₄S) (M + 1)⁺, 462.0, found 462.1. |
| Example 192 | 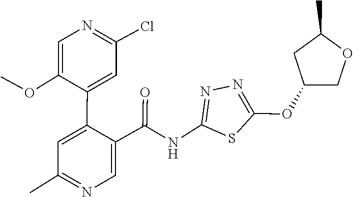 [SMILES: COc1cnc(Cl)cc1-c1cc(C)ncc1C(=O)Nc1nnc(O[C@H]2CO[C@H](C)C2)s1] 2′-chloro-5′-methoxy-6-methyl-N- (5-(((3R,5R)-5- methyltetrahydrofuran-3-yl)oxy)- 1,3,4-thiadiazol-2-yl)-(4,4′- bipyridine)-3-carboxamide. The stereochemistry was not determined. | ¹H NMR (400 MHz, DMSO-d₆) δ 12.88 (br, 1H), 8.80 (s, 1H), 8.17 (s, 1H), 7.54 (s, 1H), 7.43 (s, 1H), 5.54-5.47 (m, 1H), 4.22-4.14 (m, 1H), 4.17-4.06 (m, 1H), 3.84-3.77 (m, 1H), 3.63 (s, 3H), 2.59 (s, 3H), 2.33-2.24 (m, 1H), 1.83-1.71 (m, 1H), 1.20 (d, J = 6.0 Hz, 3H). | MS (ESI) calc′d for (C₂₀H₂₀ClN₅O₄S) (M + 1)⁺, 462.0, found 462.1. |
| Example 193 | 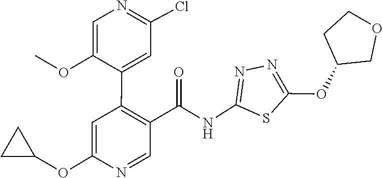 [SMILES: COc1cnc(Cl)cc1-c1cc(OC2CC2)ncc1C(=O)Nc1nnc(O[C@@H]2CCOC2)s1] (R)-2′-chloro-6-cyclopropoxy-5′- methoxy-N-(5-((tetrahydrofuran- 3-yl)oxy)-1,3,4-thiadiazol-2-yl)- (4,4′-bipyridine)-3-carboxamide (Stereochemistry determined) | ¹H NMR (400 MHz, DMSO-d₆) δ 12.83 (s, 1H), 8.61 (s, 1H), 8.16 (s, 1H), 7.54 (s, 1H), 6.99 (s, 1H), 5.54-5.47 (m, 1H), 4.40-4.31 (m, 1H), 3.96-3.81 (m, 3H), 3.81-3.71 (m, 1H), 3.64 (s, 3H), 2.33- 2.21 (m, 1H), 2.17- 2.06 (m, 1H), 0.89- 0.72 (m, 4H) | MS (ESI) calc′d for (C₂₀H₂₀ClN₅O₅S) (M + 1)⁺ 490.1; found 490.1 |
| Example 194 | 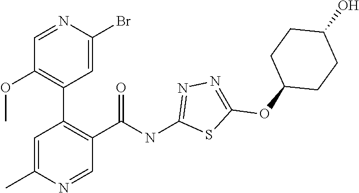 2′-bromo-N-(5-(((1r,4r)-4- hydroxycyclohexyl)oxy)-1,3,4- thiadiazol-2-yl)-5′-methoxy-6- methyl-(4,4′-bipyridine)-3- carboxamide. The relative stereochemistry is determined. | ¹H NMR (400 MHz, DMSO-d₆) δ 12.86 (s, 1H), 8.80 (s, 1H), 8.17 (s, 1H), 7.63 (s, 1H), 7.42 (s, 1H), 4.91-4.80 (m, 1H), 4.61 (d, J = 4.0 Hz, 1H), 3.65-3.60 (m, 3H), 3.59-3.50 (m, 1H), 2.59 (s, 3H), 2.18-2.05 (m, 2H), 1.88-1.79 (m, 2H), 1.62-1.48 (m, 2H), 1.40-1.27 (m, 2H). | MS (ESI) calc′d for (C₂₁H₂₂BrN₅O₄S) (M + 1)⁺, 520.1, found 520.1. |
| Example 195 | 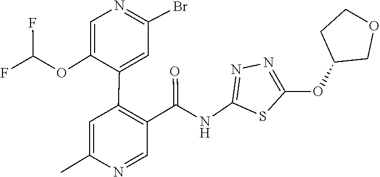 [SMILES: Cc1cc(-c2cc(Br)ncc2OC(F)F)c(C(=O)Nc2nnc(O[C@@H]3CCOC3)s2)cn1] (R)-2′-bromo-5′- (difluoromethoxy)-6-methyl-N-(5- ((tetrahydrofuran-3-yl)oxy)-1,3,4- thiadiazol-2-yl)-(4,4′-bipyridine)- 3-carboxamide. The absolute stereochemistry is determined. | ¹H NMR (400 MHz, DMSO-d₆) δ 13.07 (s, 1H), 8.95 (s, 1H), 8.32 (s, 1H), 7.83 (s, 1H), 7.45 (s, 1H), 7.31-6.95 (m, 1H), 5.53-5.48 (m, 1H), 3.96-3.81 (m, 3H), 3.81-3.74 (m, 1H), 2.61 (s, 3H), 2.33- 2.21 (m, 1H), 2.16- 2.11 (m, 1H). | MS (ESI) calculated for (C₁₉H₁₆BrF₂N₅O₄S) (M + 1)⁺, 528.0; found, 528.0. |
| Example 197 | 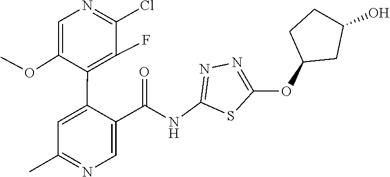 [SMILES: COc1cnc(Cl)c(F)c1-c1cc(C)ncc1C(=O)Nc1nnc(O[C@H]2CC[C@H](O)C2)s1] 2′-chloro-3′-fluoro-N-(5-(((1S,3S)- 3-hydroxycyclopentyl)oxy)-1,3,4- thiadiazol-2-yl)-5′-methoxy-6- methyl-(4,4′-bipyridine)-3- carboxamide. The stereochemistry is not determined. | ¹H NMR (400 MHz, DMSO-d₆) δ 13.01 (s, 1H), 8.97 (s, 1H), 8.18 (s, 1H), 7.47 (s, 1H), 5.36 (s, 1H), 4.66 (d, J = 3.6 Hz, 1H), 4.28-4.26 (m, 1H), 3.74 (s, 3H), 2.59 (s, 3H), 2.23- 2.13 (m, 1H), 1.99 (s, 2H), 1.94-1.82 (m, 1H), 1.80-1.77 (m, 1H), 1.59-1.49 (m, 1H). | MS (ESI) calc′d for (C₂₀H₁₉ClFN₅O₄S) (M + 1)⁺, 480.1; found, 480.1. |
| Example 198 | 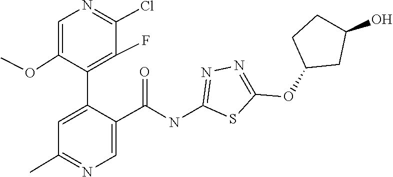 2′-chloro-3′-fluoro-N-(5- (((1R,3R)-3- hydroxycyclopentyl)oxy)-1,3,4- thiadiazol-2-yl)-5′-methoxy-6- methyl-(4,4′-bipyridine)-3- carboxamide. The stereochemistry is not determined. | ¹H NMR (400 MHz, DMSO-d₆) δ 13.01 (s, 1H), 8.97 (s, 1H), 8.18 (s, 1H), 7.47 (s, 1H), 5.36 (s, 1H), 4.66 (d, J = 3.6 Hz, 1H), 4.28-4.26 (m, 1H), 3.74 (s, 3H), 2.59 (s, 3H), 2.24- 2.14 (m, 1H), 1.99 (s, 2H), 1.93-1.71 (m, 2H), 1.59-1.49 (m, 1H). | MS (ESI) calc′d for (C₂₀H₁₉ClFN₅O₄S) (M + 1)⁺, 480.1; found, 480.1. |
| Example 199 | 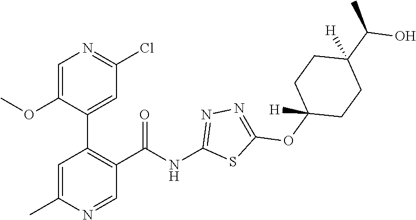 [SMILES: COc1cnc(Cl)cc1-c1cc(C)ncc1C(=O)Nc1nnc(O[C@H]2CC[C@H]([C@@H](C)O)CC2)s1] 2′-chloro-N-(5-(((1R,4r)-4-((R)-1- hydroxyethyl)cyclohexyl)oxy)- 1,3,4-thiadiazol-2-yl)-5′-methoxy- 6-methyl-(4,4′-bipyridine)-3- carboxamide. The stereochemistry is not determined. | ¹H NMR (400 MHz, DMSO-d₆) δ 12.88 (s, 1H), 8.80 (s, 1H), 8.17 (s, 1H), 7.53 (s, 1H), 7.42 (s, 1H), 4.82-4.70 (m, 1H), 4.36 (d, J = 4.8 Hz, 1H), 3.63 (s, 3H), 3.41-3.36 (m, 1H), 2.59 (s, 3H), 2.26- 2.17 (m, 2H), 1.97- 1.87 (m, 1H), 1.71- 1.69 (m, 1H), 1.50- 1.33 (m, 2H), 1.31- 1.05 (m, 3H), 1.04 (d, J = 6.4 Hz, 3H). | MS (ESI) calc′d for (C₂₃H₂₆ClN₅O₄S) (M + 1)⁺, 504.1; found, 504.1. |
| Example 200 | 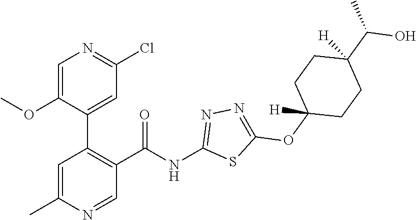 [SMILES: COc1cnc(Cl)cc1-c1cc(C)ncc1C(=O)Nc1nnc(O[C@H]2CC[C@H]([C@H](C)O)CC2)s1] 2′-chloro-N-(5-(((1S,4r)-4-((S)-1- hydroxyethyl)cyclohexyl)oxy)- 1,3,4-thiadiazol-2-yl)-5′-methoxy- 6-methyl-(4,4′-bipyridine)-3- carboxamide. The stereochemistry is not determined. | ¹H NMR (400 MHz, DMSO-d₆) δ 12.88 (s, 1H), 8.80 (s, 1H), 8.17 (s, 1H), 7.53 (s, 1H), 7.42 (s, 1H), 4.82-4.70 (m, 1H), 4.36 (d, J = 4.8 Hz, 1H), 3.63 (s, 3H), 3.41-3.36 (m, 1H), 2.59 (s, 3H), 2.26- 2.17 (m, 2H), 1.97- 1.87 (m, 1H), 1.71- 1.69 (m, 1H), 1.50- 1.33 (m, 2H), 1.31- 1.05 (m, 3H), 1.04 (d, J = 6.4 Hz, 3H). | MS (ESI) calc′d for (C₂₃H₂₆ClN₅O₄S) (M + 1)⁺, 504.1; found, 504.1. |
| Example 201 | 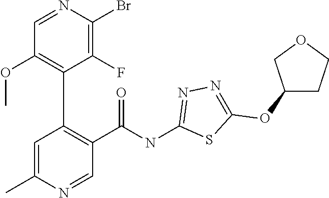 2′- bromo-3′-fluoro-5′-methoxy-6- methyl-N-(5-(((R)- tetrahydrofuran-3-yl)oxy)-1,3,4- thiadiazol-2-yl)-(4,4′-bipyridine)- 3-carboxamide. The absolute stereochemistry is determined. | ¹H NMR (400 MHz, DMSO-d₆) δ 13.06 (s, 1H), 8.97 (s, 1H), 8.20 (s, 1H), 7.47 (s, 1H), 5.51-5.49 (m, 1H), 3.90-3.84 (m, 3H), 3.84-3.73 (m, 4H), 2.60 (s, 3H), 2.34-2.20 (m, 1H), 2.16-2.08 (m, 1H). | MS (ESI) calc′d for (C₁₉H₁₇BrFN₅O₄S) (M + 1)⁺, 510.0; found, 510.1. |
| Example 202 | 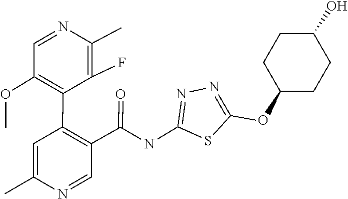 3′-fluoro-N-(5-(((1r,4r)-4- hydroxycyclohexyl)oxy)-1,3,4- thiadiazol-2-yl)-5′-methoxy-2′,6- dimethyl-(4,4′-bipyridine)-3- carboxamide. The relative stereochemistry is determined. | ¹H NMR (400 MHz, DMSO-d₆) δ 12.93 (s, 1H), 8.94 (s, 1H), 8.16 (s, 1H), 7.33 (s, 1H), 4.87-4.77 (m, 1H), 4.60 (br, 1H), 3.68 (s, 3H), 3.58- 3.48 (m, 1H), 2.57 (s, 3H), 2.42 (s, 3H), 2.15-2.04 (m, 2H), 1.83-1.80 (m, 2H), 1.58-1.48 (m, 2H), 1.39-1.23 (m, 2H). | MS (ESI) calc′d for (C₂₂H₂₄FN₅O₄S) (M + 1)⁺, 474.2; found, 474.3. |
| Example 203 | 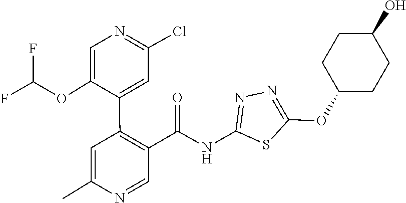 [SMILES: Cc1cc(-c2cc(Cl)ncc2OC(F)F)c(C(=O)Nc2nnc(O[C@H]3CC[C@H](O)CC3)s2)cn1] 2′-chloro-5′-(difluoromethoxy)-N- (5-(((1r,4r)-4- hydroxycyclohexyl)oxy)-1,3,4- thiadiazol-2-yl)-6-methyl-(4,4′- bipyridine)-3-carboxamide. The relative stereochemistry is determined. | ¹H NMR (400 MHz, DMSO-d₆) δ 13.01 (s, 1H), 8.96 (s, 1H), 8.33 (s, 1H), 7.72 (s, 1H), 7.45 (s, 1H), 7.14-6.95 (m, 1H), 4.85-4.82 (m, 1H), 4.61 (d, J = 4.0 Hz, 1H), 3.55-3.54 (m, 1H), 2.61 (s, 3H), 2.16-2.06 (m, 2H), 1.83-1.80 (m, 2H), 1.55-1.50 (m, 2H), 1.40-1.27 (m, 2H). | MS (ESI) calculated for (C₂₁H₂₀ClF₂N₅O₄S) (M + 1)⁺, 512.1; found, 512.1. |
| Example 204 | 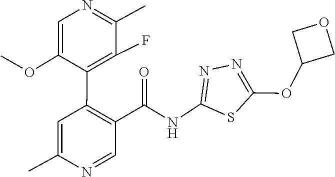 [SMILES: COc1cnc(C)c(F)c1-c1cc(C)ncc1C(=O)Nc1nnc(OC2COC2)s1] (2′-bromo-N-(5-(((1r,4r)-4- hydroxycyclohexyl)oxy)-1,3,4- thiadiazol-2-yl)-5′-methoxy-6- methyl-(4,4′-bipyridine)-3- carboxamide | ¹H NMR (400 MHz, DMSO-d₆) δ 13.09 (s, 1H), 8.91 (s, 1H), 8.18 (s, 1H), 7.39 (s, 1H), 5.71-5.61 (m, 1H), 4.93-4.85 (m, 2H), 4.68-4.59 (m, 2H), 3.68 (s, 3H), 2.59 (s, 3H), 2.43 (s, 3H). | (ESI) calc′d for (C₁₉H₁₈FN₅O₄S) (M + 1)⁺, 432.1; found, 432.1. |
| Example 205 | 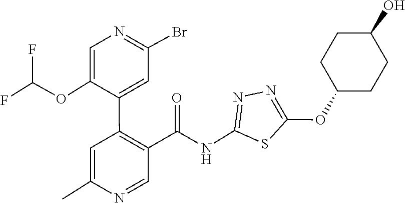 [SMILES: Cc1cc(-c2cc(Br)ncc2OC(F)F)c(C(=O)Nc2nnc(O[C@H]3CC[C@H](O)CC3)s2)cn1] 2′-bromo-5′-(difluoromethoxy)-N- (5-(((1r,4r)-4- hydroxycyclohexyl)oxy)-1,3,4- thiadiazol-2-yl)-6-methyl-(4,4′- bipyridine)-3-carboxamide. The relative stereochemistry is determined. | ¹H NMR (400 MHz, DMSO-d₆) δ 13.03 (s, 1H), 8.95 (s, 1H), 8.32 (s, 1H), 7.82 (s, 1H), 7.44 (s, 1H), 7.32-6.85 (m, 1H), 4.93-4.82 (s, 1H), 4.61 (d, J = 4.4 Hz, 1H), 3.59-3.52 (m, 1H), 2.61 (s, 3H), 2.13-2.09 (m, 2H), 1.85-1.81 (m, 2H), 1.62-1.48 (m, 2H), 1.40-1.26 (m, 2H). | MS (ESI) calculated for (C₂₁H₂₀BrF₂N₅O₄S) (M + 1)⁺, 556.0, 558.0; found, 556.0, 558.0. |
| Example 206 | 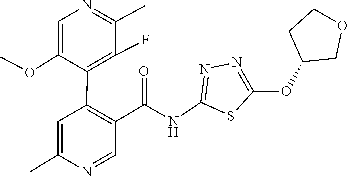 [SMILES: COc1cnc(C)c(F)c1-c1cc(C)ncc1C(=O)Nc1nnc(O[C@@H]2CCOC2)s1] (R)-6-(2,4-difluorophenyl)-N- (1,1-dioxido-2,3-dihydrothiophen- 3-yl)-2-oxo-1,2-dihydropyridine- 3-carboxamide. The absolute stereochemistry is determined. | ¹H NMR (400 MHz, DMSO-d₆) δ 12.99 (s, 1H), 8.91 (s, 1H), 8.18 (s, 1H), 7.41 (s, 1H), 5.54-5.47 (m, 1H), 3.95-3.82 (m, 3H), 3.81-3.71 (m, 1H), 3.69 (s, 3H), 2.59 (s, 3H), 2.43 (s, 3H), 2.33-2.21 (m, 1H), 2.17-2.07 (m, 1H). | MS (ESI) calc′d for (C₂₀H₂₀FN₅O₄S) (M + 1)⁺, 446.1; found, 446.1 |
| Example 209 | 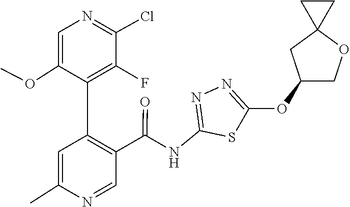 [SMILES: COc1cnc(Cl)c(F)c1-c1cc(C)ncc1C(=O)Nc1nnc(O[C@@H]2COC3(CC3)C2)s1] N-(5-(((S)-4-oxaspiro(2.4)heptan- 6-yl)oxy)-1,3,4-thiadiazol-2-yl)- 2′-chloro-3′-fluoro-5′-methoxy-6- methyl-(4,4′-bipyridine)-3- carboxamide. The stereochemistry was not determined. | ¹H NMR (400 MHz, DMSO-d₆) δ 13.05 (s, 1H), 8.97 (s, 1H), 8.19 (s, 1H), 7.49 (s, 1H), 5.64-5.56 (m, 1H), 4.09-3.94 (m, 2H), 3.74 (s, 3H), 2.60 (s, 3H), 2.51- 2.43 (m, 1H), 2.19- 2.11 (m, 1H), 0.88- 0.77 (m, 1H), 0.77- 0.68 (m, 1H), 0.66- 0.56 (m, 1H), 0.55- 0.46 (m, 1H). | MS (ESI) calc′d for (C₂₁H₁₉ClFN₅O₄S) (M + 1)⁺, 492.1, found 492.1. |
| Example 210 | 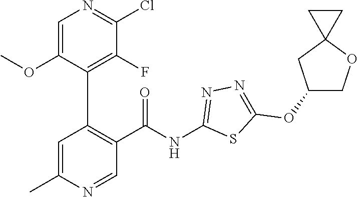 [SMILES: COc1cnc(Cl)c(F)c1-c1cc(C)ncc1C(=O)Nc1nnc(O[C@H]2COC3(CC3)C2)s1] N-(5-(((R)-4-oxaspiro(2.4)heptan- 6-yl)oxy)-1,3,4-thiadiazol-2-yl)- 2′-chloro-3′-fluoro-5′-methoxy-6- methyl-(4,4′-bipyridine)-3- carboxamide. The stereochemistry was not determined. | ¹H NMR (400 MHz, DMSO-d₆) δ 13.06 (s, 1H), 8.98 (s, 1H), 8.18 (s, 1H), 7.47 (s, 1H), 5.63-5.55 (m, 1H), 4.10-3.94 (m, 2H), 3.74 (s, 3H), 2.60 (s, 3H), 2.50- 2.42 (m, 1H), 2.18- 2.10 (m, 1H), 0.87- 0.77 (m, 1H), 0.77- 0.68 (m, 1H), 0.66- 0.56 (m, 1H), 0.55- 0.45 (m, 1H). | MS (ESI) calc′d for (C₂₁H₁₉ClFN₅O₄S) (M + 1)⁺, 492.1, found 492.1. |
| Example 211 | 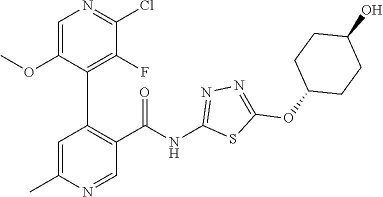 [SMILES: COc1cnc(Cl)c(F)c1-c1cc(C)ncc1C(=O)Nc1nnc(O[C@H]2CC[C@H](O)CC2)s1] 2′-chloro-3′-fluoro-N-(5-(((1r,4r)- 4-hydroxycyclohexyl)oxy)-1,3,4- thiadiazol-2-yl)-5′-methoxy-6- methyl-(4,4′-bipyridine)-3- carboxamide. The relative stereochemistry is determined. | ¹H NMR (400 MHz, DMSO-d₆) δ 13.01 (s, 1H), 8.96 (s, 1H), 8.19 (s, 1H), 7.48 (s, 1H), 4.91-4.80 (m, 1H), 3.74 (s, 3H), 3.60-3.49 (m, 1H), 2.60 (s, 3H), 2.17- 2.06 (m, 2H), 1.91- 1.76 (m, 2H), 1.62- 1.48 (m, 2H), 1.40- 1.22 (m, 2H). | MS (ESI) calculated for (C₂₁H₂₁ClFN₅O₄S) (M + 1)⁺, 494.1; found, 494.1. |
| Example 212 | 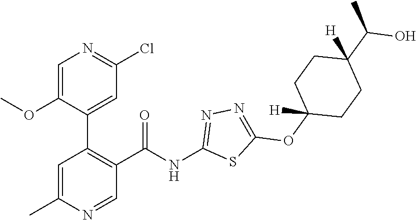 [SMILES: COc1cnc(Cl)cc1-c1cc(C)ncc1C(=O)Nc1nnc(O[C@H]2CC[C@@H]([C@@H](C)O)CC2)s1] 2′-chloro-N-(5-(((1S,4s)-4-((R)-1- hydroxyethyl)cyclohexyl)oxy)- 1,3,4-thiadiazol-2-yl)-5′-methoxy- 6-methyl-(4,4′-bipyridine)-3- carboxamide. The stereochemistry was not determined. | ¹H NMR (400 MHz, DMSO-d₆) δ 12.84 (s, 1H), 8.81 (s, 1H), 8.17 (s, 1H), 7.53 (s, 1H), 7.42 (s, 1H), 5.08 (s, 1H), 4.32 (d, J = 4.8 Hz, 1H), 3.64 (s, 3H), 3.45-3.37 (m, 1H), 2.59 (s, 3H), 2.09-2.07 (m, 2H), 1.69-1.55 (m, 3H), 1.51-1.43 (m, 1H), 1.40-1.21 (m, 3H), 1.04 (d, J = 6.4 Hz, 3H). | MS (ESI) calc′d for (C₂₃H₂₆ClN₅O₄S) (M + 1)⁺, 504.1; found, 504.1. |
| Example 213 | 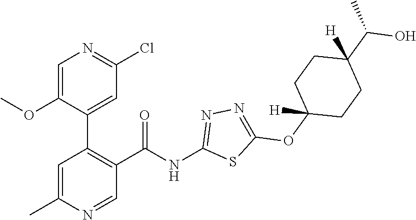 [SMILES: COc1cnc(Cl)cc1-c1cc(C)ncc1C(=O)Nc1nnc(O[C@H]2CC[C@@H]([C@H](C)O)CC2)s1] 2′-chloro-N-(5-(((1R,4s)-4-((S)-1- hydroxyethyl)cyclohexyl)oxy)- 1,3,4-thiadiazol-2-yl)-5′-methoxy- 6-methyl-(4,4′-bipyridine)-3- carboxamide. The stereochemistry was not determined. | ¹H NMR (400 MHz, DMSO-d₆) δ 12.83 (s, 1H), 8.81 (s, 1H), 8.17 (s, 1H), 7.53 (s, 1H), 7.42 (s, 1H), 5.08 (s, 1H), 4.32 (d, J = 4.8 Hz, 1H), 3.64 (s, 3H), 3.45-3.37 (m, 1H), 2.59 (s, 3H), 2.09-2.07 (m, 2H), 1.70-1.52 (m, 3H), 1.51-1.43 (m, 1H), 1.41-1.19 (m, 3H), 1.04 (d, J = 6.4 Hz, 3H) | MS (ESI) calc′d for (C₂₃H₂₆ClN₅O₄S) (M + 1)⁺, 504.1; found, 504.1. |
| Example 214 | 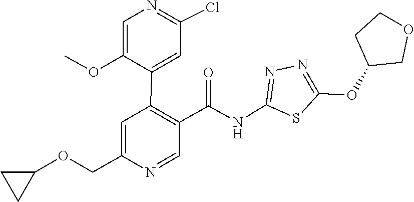 [SMILES: COc1cnc(Cl)cc1-c1cc(COC2CC2)ncc1C(=O)Nc1nnc(O[C@@H]2CCOC2)s1] (R)-2′-chloro-6- (cyclopropoxymethyl)-5′- methoxy-N-(5-((tetrahydrofuran- 3-yl)oxy)-1,3,4-thiadiazol-2-yl)- (4,4′-bipyridine)-3-carboxamide. The absolute stereochemistry is determined. | ¹H NMR (400 MHz, DMSO-d₆) δ 12.97 (s, 1H), 8.89 (s, 1H), 8.19 (s, 1H), 7.55 (s, 1H), 7.48 (s, 1H), 5.56-5.50 (m, 1H), 4.70 (s, 2H), 3.93- 3.81 (m, 3H), 3.77- 3.75 (m, 1H), 3.64 (s, 3H), 3.53-3.51 (m, 1H), 2.33-2.21 (m, 1H), 2.15-2.11 (m, 1H), 0.61-0.59 (m, 2H), 0.52-0.49 (m, 2H). | MS (ESI) calculated for (C₂₂H₂₂ClN₅O₂S) (M + 1)⁺, 504.1; found, 504.1. |
| Example 220 | 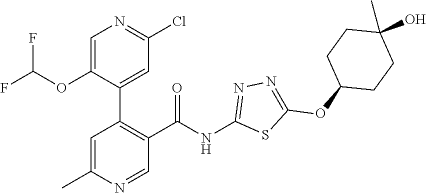 [SMILES: Cc1cc(-c2cc(Cl)ncc2OC(F)F)c(C(=O)Nc2nnc(O[C@H]3CC[C@@](C)(O)CC3)s2)cn1] 2′-chloro-5′-(difluoromethoxy)-N- (5-(((1s,4s)-4-hydroxy-4- methylcyclohexyl)oxy)-1,3,4- thiadiazol-2-yl)-6-methyl-(4,4′- bipyridine)-3-carboxamide. The relative stereochemistry was not determined. | 1H-NMR (400 MHz, DMSO-d⁶): δ 13.02 (s, 1H), 8.95 (s, 1H), 8.33 (s, 1H), 7.73 (s, 1H), 7.45 (s, 1H), 7.14 (t, J = 72.0 Hz, 1H), 4.89-4.77 (m, 1H), 4.23 (s, 1H), 2.61 (s, 3H), 1.95- 1.76 (m, 4H), 1.67- 1.55 (m, 2H), 1.50 | MS (ESI) calculated for (C₂₂H₂₂ClF₂N₅O₄S) (M + 1)⁺, 526.11; found, 526.1. |
| Example 221 | 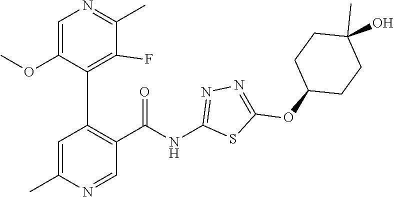 [SMILES: COc1cnc(C)c(F)c1-c1cc(C)ncc1C(=O)Nc1nnc(O[C@H]2CC[C@@](C)(O)CC2)s1] 3′-fluoro-N-(5-(((1s,4s)-4- hydroxy-4- methylcyclohexyl)oxy)-1,3,4- thiadiazol-2-yl)-5′-methoxy-2′,6- dimethyl-(4,4′-bipyridine)-3- carboxamide. The relative stereochemistry was not determined. | 1H-NMR (400 MHz, DMSO-d⁶): δ 12.97 (s, 1H), 8.92 (s, 1H), 8.17 (s, 1H), 7.37 (s, 1H), 4.85-4.75 (m, 1H), 4.22 (s, 1H), 3.69 (s, 3H), 2.58 (s, 3H), 2.42 (d, J = 3.2 Hz, 3H), 1.91-1.76 (m, 4H), 1.66-1.57 (m, 2H), 1.47-1.37 (m, 2H), 1.12 (s, 3H). | MS (ESI) calculated for (C₂₃H₂₆FN₅O₄S) (M + 1)⁺, 488.18; found, 488.2. |
| Example 222 | 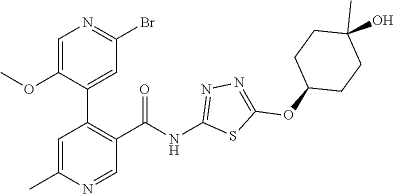 [SMILES: COc1cnc(Br)cc1-c1cc(C)ncc1C(=O)Nc1nnc(O[C@H]2CC[C@@](C)(O)CC2)s1] 2′-bromo-N-(5-(((1s,4s)-4- hydroxy-4- methylcyclohexyl)oxy)-1,3,4- thiadiazol-2-yl)-5′-methoxy-6- methyl-(4,4′-bipyridine)-3- carboxamide. The relative stereochemistry was not determined. | 1H-NMR (400 MHz, DMSO-d⁶): δ 12.87 (s, 1H), 8.83 (s, 1H), 8.16 (s, 1H), 7.59 (s, 1H), 7.37 (s, 1H), 4.84-4.74 (m, 1H), 4.22 (s, 1H), 3.62 (s, 3H), 2.58 (s, 3H), 1.92-1.77 (m, 4H), 1.65-1.57 (m, 2H), 1.47-1.37 (m, 2H), 1.12 (s, 3H). | MS (ESI) calculated for (C₂₂H₂₄BrN₅O₄S) (M + 1)⁺, 534.08; found, 534.0. |
| Example 223 | 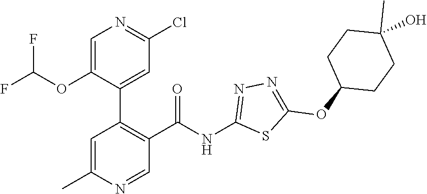 [SMILES: Cc1cc(-c2cc(Cl)ncc2OC(F)F)c(C(=O)Nc2nnc(O[C@H]3CC[C@](C)(O)CC3)s2)cn1] 2′-chloro-5′-(difluoromethoxy)-N- (5-(((1r,4r)-4-hydroxy-4- methylcyclohexyl)oxy)-1,3,4- thiadiazol-2-yl)-6-methyl-(4,4′- bipyridine)-3-carboxamide. The relative stereochemistry was not determined. | 1H-NMR (400 MHz, DMSO-d6): δ 13.03 (s, 1H), 8.95 (s, 1H), 8.33 (s, 1H), 7.72 (s, 1H), 7.45 (s, 1H), 7.14 (t, J = 71.4 Hz, 1H), 5.05-4.97 (m, 1H), 4.23 (s, 1H), 2.61 (s, 3H), 2.02- 1.90 (m, 2H), 1.84- 1.72 (m, 2H), 1.62- 1.50 (m, 2H), 1.49- 1.38 (m, 2H), 1.15 (s, 3H). | MS (ESI) calculated for (C₂₂H₂₂ClF₂N₅O₄S) (M + 1)⁺, 526.11; found, 526.0 |
| Example 224 | 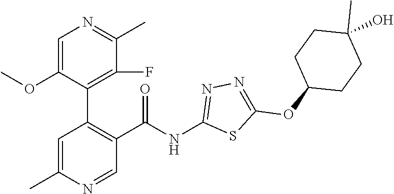 [SMILES: COc1cnc(C)c(F)c1-c1cc(C)ncc1C(=O)Nc1nnc(O[C@H]2CC[C@](C)(O)CC2)s1] 3′-fluoro-N-(5-(((1r,4r)-4- hydroxy-4- methylcyclohexyl)oxy)-1,3,4- thiadiazol-2-yl)-5′-methoxy-2′,6- dimethyl-(4,4′-bipyridine)-3- carboxamide. The relative stereochemistry was not determined. | 1H-NMR (400 MHz, DMSO-d⁶): δ 12.94 (s, 1H), 8.91 (s, 1H), 8.18 (s, 1H), 7.38 (s, 1H), 5.02-4.95 (m, 1H), 4.22 (s, 1H), 3.69 (s, 3H), 2.58 (s, 3H), 2.42 (d, J = 3.20 Hz, 3H), 2.02-1.90 (m, 2H), 1.81-1.70 (m, 2H), 1.60-1.50 (m, 2H), 1.48-1.39 (m, 2H), 1.14 (s, 3H). | MS (ESI) calculated for (C₂₃H₂₆FN₅O₄S) (M − 1)⁻, 486.16; found, 486.1. |
| Example 225 | 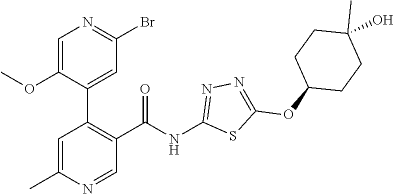 [SMILES: COc1cnc(Br)cc1-c1cc(C)ncc1C(=O)Nc1nnc(O[C@H]2CC[C@](C)(O)CC2)s1] 2′-bromo-N-(5-(((1r,4r)-4- hydroxy-4- methylcyclohexyl)oxy)-1,3,4- thiadiazol-2-yl)-5′-methoxy-6- methyl-(4,4′-bipyridine)-3- carboxamide. The relative stereochemistry was not determined. | 1H-NMR (400 MHz, DMSO-d⁶): δ 12.87 (s, 1H), 8.80 (s, 1H), 8.18 (s, 1H), 7.63 (s, 1H), 7.42 (s, 1H), 5.06-4.97 (m, 1H), 4.23 (s, 1H), 3.63 (s, 3H), 2.59 (s, 3H), 2.03-1.90 (m, 2H), 1.85-1.73 (m, 2H), 1.62-1.50 (m, 2H), 1.49-1.38 (m, 2H), 0.58 (s, 3H). | MS (ESI) calculated for (C₂₂H₂₄BrN₅O₄S) (M + 1)⁺, 534.08; found, 534.0. |
| Example 232 | 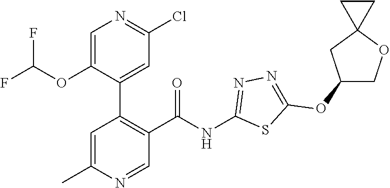 [SMILES: Cc1cc(-c2cc(Cl)ncc2OC(F)F)c(C(=O)Nc2nnc(O[C@@H]3COC4(CC4)C3)s2)cn1] (S)-N-(5-((4-oxaspiro(2.4)heptan- 6-yl)oxy)-1,3,4-thiadiazol-2-yl)- 2′-chloro-5′-(difluoromethoxy)-6- methyl-(4,4′-bipyridine)-3- carboxamide. The absolute stereochemistry is not determined. | ¹H-NMR (400 MHz, DMSO-d⁶): δ 13.14 (s, 1H), 8.98 (s, 1H), 8.32 (s, 1H), 7.70 (s, 1H), 7.42 (s, 1H), 7.14 (t, J = 72.8 Hz, 1H), 5.61-5.55 (m, 1H), 4.02 (dd, J = 4.4 Hz, 10.4 Hz, 1H), 3.97 (d, J = 10.0 Hz, 1H), 2.60 (s, 3H), 2.50-2.40 (m, 1H), 2.13 (d, J = 13.6 Hz, 1H), 0.85-0.78 (m, 1H), 0.77-0.69 (m, 1H), 0.65-0.57 (m, 1H), 0.54-0.46 (m, 1H). | MS (ESI) calculated for (C₂₁H₁₈ClF₂N₅O₄S) (M + 1)⁺, 510.08; found, 510.0. |
| Example 233 | 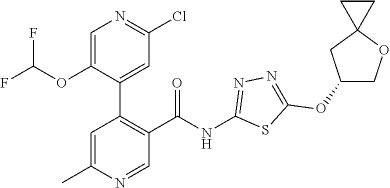 [SMILES: Cc1cc(-c2cc(Cl)ncc2OC(F)F)c(C(=O)Nc2nnc(O[C@H]3COC4(CC4)C3)s2)cn1] (R)-N-(5-((4-oxaspiro(2.4)heptan- 6-yl)oxy)-1,3,4-thiadiazol-2-yl)- 2′-chloro-5′-(difluoromethoxy )-6- methyl-(4,4′-bipyridine)-3- carboxamide. The absolute stereochemistry is not determined. | ¹H-NMR (400 MHz, DMSO-d⁶): δ 13.14 (s, 1H), 8.97 (s, 1H), 8.33 (s, 1H), 7.72 (s, 1H), 7.44 (s, 1H), 7.15 (t, J = 72.4 Hz, 1H), 5.62-5.55 (m, 1H), 4.02 (dd, J = 4.8 Hz, 10.8 Hz, 1H), 3.97 (d, J = 10.0 Hz, 1H), 2.60 (s, 3H), 2.50-2.40 (m, 1H), 2.13 (d, J = 14.0 Hz, 1H), 0.86-0.78 (m, 1H), 0.77-0.69 (m, 1H), 0.65-0.56 (m, 1H), 0.53-0.46 (m, 1H) | MS (ESI) calculated for (C₂₁H₁₈ClF₂N₅O₄S) (M + 1)⁺, 510.08; found, 510.0. |
| Example 236 | 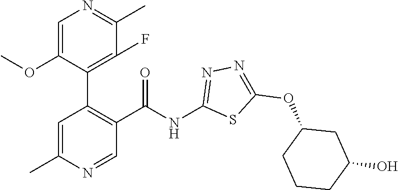 [SMILES: COc1cnc(C)c(F)c1-c1cc(C)ncc1C(=O)Nc1nnc(O[C@H]2CCC[C@@H](O)C2)s1] 3′-fluoro-N-(5-(((1S,3R)-3- hydroxycyclohexyl)oxy)-1,3,4- thiadiazol-2-yl)-5′-methoxy-2′,6- dimethyl-[4,4′-bipyridine]-3- carboxamide. The absolute stereochemistry was not determined and the relative stereochemistry was determined by X-ray crystallography. | 1H-NMR (400 MHz, DMSO-d⁶): δ 12.96 (s, 1H), 8.90 (s, 1H), 8.18 (s, 1H), 7.39 (s, 1H), 4.86-4.75 (m, 2H), 3.69 (s, 3H), 3.55-3.46 (m, 1H), 2.59 (s, 3H), 2.43 (d, J = 3.2 Hz, 3H) 2.43- 2.38 (m, 1H), 2.15- 2.08 (m, 1H), 1.83- 1.68 (m, 2H), 1.35- 1.20 (m, 3H), 1.15- 1.03 (m, 1H). | MS (ESI) calculated for (C₂₂H₂₄FN₅O₄S) (M + 1)⁺, 474.16; found, 474.1. |
| Example 237 | 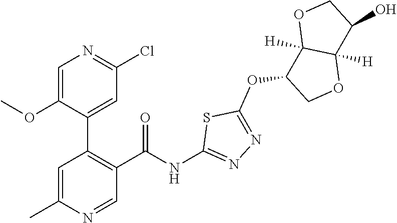 [SMILES: COc1cnc(Cl)cc1-c1cc(C)ncc1C(=O)Nc1nnc(O[C@H]2CO[C@H]3[C@@H]2OC[C@H]3O)s1] 2′-chloro-N-(5- (((3S,3aR,6R,6aR)-6- hydroxyhexahydrofuro[3,2- b]furan-3-yl)oxy)-1,3,4- thiadiazol-2-yl)-5′-methoxy-6- methyl-[4,4′-bipyridine]-3- carboxamide | ¹H-NMR (400 MHz, DMSO-d₆): δ 13.13- 12.84 (m, 1H), 8.81 (s, 1H), 8.16 (s, 1H), 7.53 (s, 1H), 7.47- 7.33 (m, 1H), 5.29 (d, J = 3.4 Hz, 1H), 4.98 (d, J = 6.4 Hz, 1H), 4.63 (d, J = 4.4 Hz, 1H), 4.51 (t, J = 4.9 Hz, 1H), 4.20-4.12 (m, 1H), 4.12-4.06 (m, 1H), 4.05-3.98 (m, 1H), 3.77 (dd, J = 8.8, 6.4 Hz, 1H), 3.62 (s, 3H), 3.40 (dd, J = 8.6, 7.6 Hz, 1H), 2.58 (s, 3H) | MS (ESI) calc′d for (C₂₁H₂₀ClN₅O₆S) (M + 1)⁺, 506.0, found 506.1 |
| Example 238 | 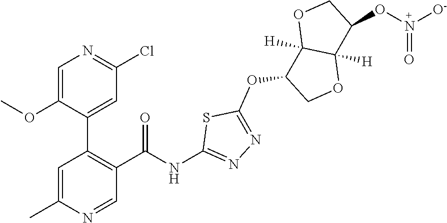 (3R,3aS,6S,6aR)-6-((5-(2′-chloro- 5′-methoxy-6-methyl-[4,4′- bipyridine]-3-carboxamido)-1,3,4- thiadiazol-2- yl)oxy)hexahydrofuro[3,2- b]furan-3-yl nitrate | ¹H NMR (400 MHz, CHLOROFORM-d) δ 12.74 (br s, 1H), 8.99 (s, 1H), 8.04 (s, 1H), 7.30 (s, 1H), 7.21 (s, 1H), 5.45-5.39 (m, 2H), 5.09 (t, J = 5.4 Hz, 1H), 4.70 (d, J = 4.9 Hz, 1H), 4.25 (d, J = 11.7 Hz, 1H), 4.13- 4.03 (m, 2H), 4.00- 3.94 (m, 1H), 3.73 (s, 3H), 2.72 (s, 3H). | MS (ESI) calc′d for (C₂₁H₁₉ClN₆O₈S) (M + 1)⁺, 551.0, found 551.1. |
| Example 239 | 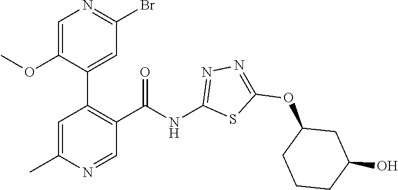 [SMILES: COc1cnc(Br)cc1-c1cc(C)ncc1C(=O)Nc1nnc(O[C@@H]2CCC[C@H](O)C2)s1] 2′-bromo-N-(5-(((1R,3S)-3- hydroxycyclohexyl)oxy)-1,3,4- thiadiazol-2-yl)-5′-methoxy-6- methyl-[4,4′-bipyridine]-3- carboxamide. The absolute stereochemistry was not determined and the relative stereochemistry was determined by X-ray crystallography. | 1H-NMR (400 MHz, DMSO-d⁶): δ 12.90 (s, 1H), 8.84 (s, 1H), 8.16 (s, 1H), 7.59 (s, 1H), 7.37 (s, 1H), 4.78 (d, J = 4.80 Hz, 2H), 3.63 (s, 3H), 3.55- 3.42 (m, 1H), 2.57 (s, 3H), 2.43-2.34 (m, 1H), 2.15-2.06 (m, 1H), 1.83-1.67 (m, 2H), 1.35-1.20 (m, 3H), 1.18-1.00 (m, 1H). | MS (ESI) calculated for (C₂₁H₂₂BrN₅O₄S) (M + 1)⁺, 520.0; found, 519.8 . |
| Example 240 | 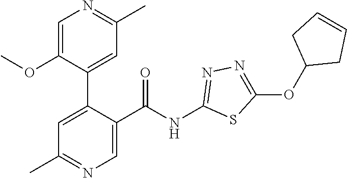 [SMILES: COc1cnc(C)cc1-c1cc(C)ncc1C(=O)Nc1nnc(OC2CC=CC2)s1] N-(5-(cyclopent-3-en-1-yloxy)- 1,3,4-thiadiazol-2-yl)-5′-methoxy- 2′,6-dimethyl-[4,4′-bipyridine]-3- carboxamide. | ¹H NMR (700 MHz, DMSO-d₆) δ ppm 12.85 (br s, 1 H), 8.75 (s, 1 H), 8.18 (s, 1 H), 7.36 (s, 1 H), 7.25 (s, 1 H), 5.78 (s, 2 H), 5.54 (t, J = 6.00 Hz, 1 H), 3.58 (s, 3 H), 2.83 (dd, J = 17.20, 6.50 Hz, 2 H), 2.58 (s, 3 H), 2.53 (d, J = 17.60 Hz, 2 H), 2.47 (s, 3 H). | MS (ESI) calc′d for (C₂₁H₂₁N₅O₃S) (M + 1)⁺, 424.49, found 424.3 |
| Example 241 | 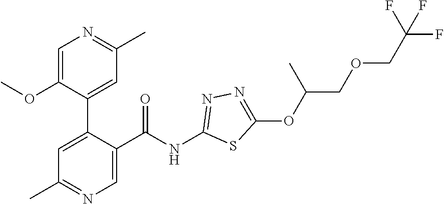 [SMILES: COc1cnc(C)cc1-c1cc(C)ncc1C(=O)Nc1nnc(OC(C)COCC(F)(F)F)s1] 5′-methoxy-2′,6-dimethyl-N-(5- ((1-(2,2,2-trifluoroethoxy)propan- 2-yl)oxy)-1,3,4-thiadiazol-2-yl)- [4,4′-bipyridine]-3-carboxamide. | ¹H NMR (700 MHz, DMSO-d₆) δ ppm 12.86 (br s, 1 H), 8.75 (br s, 1 H), 8.19 (s, 1 H), 7.37 (s, 1 H), 7.26 (s, 1 H), 5.18-5.23 (m, 1 H), 4.13 (qd, J = 9.39, 2.37 Hz, 2 H), 3.85 (dd, J = 11.00, 3.50 Hz, 1 H), 3.81 (dd, J = 11.00, 6.50 Hz, 1 H), 3.59 (s, 3 H), 2.58 (s, 3 H), 2.47 (s, 3 H), 1.35 (d, J = 6.45 Hz, 3 H). | MS (ESI) calc′d for (C₂₁H₂₂F₃N₅O₄S) (M + 1)⁺, 498.49, found 498.2. |
| Example 244 | 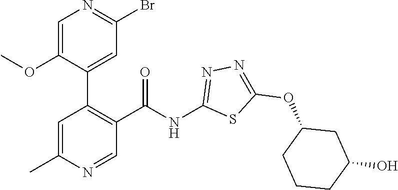 [SMILES: COc1cnc(Br)cc1-c1cc(C)ncc1C(=O)Nc1nnc(O[C@H]2CCC[C@@H](O)C2)s1] 2′-bromo-N-(5-(((1S,3R)-3- hydroxycyclohexyl)oxy)-1,3,4- thiadiazol-2-yl)-5′-methoxy-6- methyl-[4,4′-bipyridine]-3- carboxamide. The absolute stereochemistry was not determined and the relative stereochemistry was determined by X-ray crystallography. | 1H-NMR (400 MHz, DMSO-d6): δ 12.88 (s, 1H), 8.79 (s, 1H), 8.18 (s, 1H), 7.64 (s, 1H), 7.43 (s, 1H), 4.90-4.77 (m, 2H), 3.63 (s, 3H), 3.57- 3.45 (m, 1H), 2.59 (s, 3H), 2.45-2.37 (m, 1H), 2.18-2.06 (m, 1H), 1.86-1.77 (m, 1H), 1.77-1.68 (m, 1H), 1.38-1.22 (m, 3H), 1.17-1.03 (m, 1H), | MS (ESI) calculated for (C₂₁H₂₂BrN₅O₄S) (M + 1)⁺, 520.07; found, 520.0. |
| Example 245 | 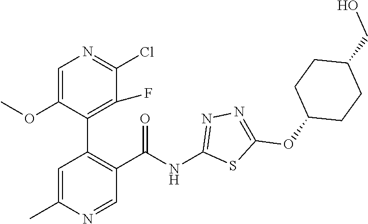 [SMILES: COc1cnc(Cl)c(F)c1-c1cc(C)ncc1C(=O)Nc1nnc(O[C@H]2CC[C@@H](CO)CC2)s1] 2′-chloro-3′-fluoro-N-(5-(((1s,4s)- 4- (hydroxymethyl)cyclohexyl)oxy)- 1,3,4-thiadiazol-2-yl)-5′-methoxy- 6-methyl-[4,4′-bipyridine]-3- carboxamide | 1H-NMR (400 MHz, DMSO-d⁶): δ 13.00 (brs, 1H), 8.98 (s, 1H), 8.18 (s, 1H), 7.46 (s, 1H), 5.11- 5.03 (m, 1H), 4.43 (t, J = 5.2 Hz, 1H), 3.75 (s, 3H), 3.25-3.15 (m, 2H), 2.59 (s, 3H), 2.08-1.97 (m, 2H), 1.70-1.50 (m, 5H), 1.35-1.20 (m, 2H) | MS (ESI) calculated for (C₂₂H₂₃ClFN₅O₄S) (M + 1)⁺, 508.12; found, 508.0 |
| Example 246 | 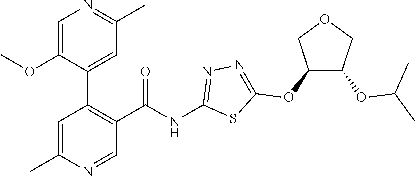 [SMILES: COc1cnc(C)cc1-c1cc(C)ncc1C(=O)Nc1nnc(O[C@H]2COC[C@@H]2OC(C)C)s1] N-(5-(((3R,4S)-4- isopropyltetrahydrofuran-3- yl)oxy)-1,3,4-thiadiazol-2-yl)-5′- methoxy-2′,6-dimethyl-[4,4′- bipyridine]-3-carboxamide. The relative stereochemistry was determined as synthesis used starting material with known relative sterochemistry.The absolute stereochemistry was not determined. | ¹H NMR (700 MHz, DMSO-d₆) δ ppm 12.90 (br s, 1 H), 8.75 (s, 1 H), 8.19 (s, 1 H), 7.37 (s, 1 H), 7.26 (s, 1 H) 5.28 (d, J = 3.44 Hz, 1 H), 4.29-4.31, (m, 1 H), 3.96-4.01 (m, 2 H), 3.92 (d, J = 11.19 Hz, 1 H), 3.87 (quin, J = 6.00 Hz, 1 H), 3.60 (d, J = 2.58 Hz, 1 H), 3.59 (s, 3 H), 2.59 (s, 3 H), 2.47 (s, 3 H), 1.14 (d, J = 6.02 Hz, 3 H), 1.11 (d, J = 6.02 Hz, 3 H). | MS (ESI) calculated for (C₂₃H₂₇N₅O₄S) (M + 1)⁺, 486.56; found, 486.3. |
| Example 247 | 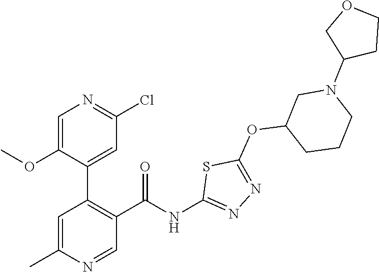 [SMILES: COc1cnc(Cl)cc1-c1cc(C)ncc1C(=O)Nc1nnc(OC2CCCN(C3CCOC3)C2)s1] 2′-chloro-5′-methoxy-6-methyl-N- (5-((1-(tetrahydrofuran-3- yl)piperidin-3-yl)oxy)-1,3,4- thiadiazol-2-yl)-[4,4′-bipyridine]- 3-carboxamide | | MS (ESI) calc′d for (C₂₄H₂₇ClN₆O₄S) (M + 1)⁺, 531.0, found 531.2 |
| Example 248. | 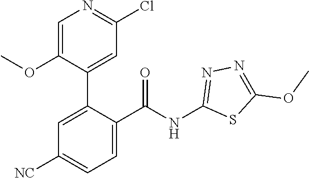 2-(2-chloro-5-methoxypyridin-4- yl)-4-cyano-N-(5-methoxy-1,3,4- thiadiazol-2-yl)benzamide | ¹H NMR (DMSO-d₆, 400 MHz) δ 12.97 (br s, 1H), 8.14 (s, 1H), 8.09 (dd, 1H, J = 1.5, 8.3 Hz), 8.03 (d, 1H, J = 1.0 Hz), 7.94 (d, 1H, J = 8.3 Hz), 7.58 (s, 1H), 4.07 (s, 3H), 3.61 (s, 3H) | MS (ESI) calc′d for (C₁₇H₁₂ClN₅O₃S) (M + 1)⁺, 402.0, found 402.0. |
| Example 249 | 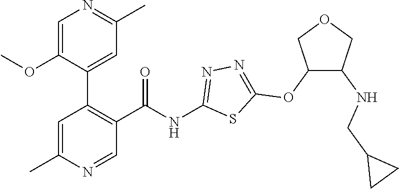 [SMILES: COc1cnc(C)cc1-c1cc(C)ncc1C(=O)Nc1nnc(OC2COCC2NCC2CC2)s1] N-(5-((4- ((cyclopropylmethyl)amino) tetrahydrofuran-3-yl)oxy)-1,3,4- thiadiazol-2-yl)-5′-methoxy-2′,6- dimethyl-[4,4′-bipyridine]-3- carboxamide. | ¹H NMR (700 MHz, DMSO-d₆) δ ppm 12.32 (br s, 1 H), 8.75 (s, 1 H), 8.17-8.20 (m, 1 H), 7.36 (s, 1 H), 7.25 (s, 1 H), 5.20- 5.25 (m, 1 H), 4.04 (dd, J = 8.82, 5.81 Hz, 1 H), 4.00 (dd, J = 10.76, 4.30 Hz, 1 H), 3.91 (d, J = 10.76 Hz, 1 H), 3.67 (s, 1 H), 3.58 (s, 3 H), 3.43- 3.52 (m, 2 H), 2.58 (s, 3 H), 2.47 (s, 3 H), 2.08 (s, 2 H), 0.84- 0.93 (m, 1 H), 0.37- 0.43 (m, 2 H), 0.10- 0.15 (m, 2 H). | MS (ESI) calculated for (C₂₄H₂₈N₆O₄S) (M + 1)⁺, 497.19; found, 497.3. |
| Example 250 | 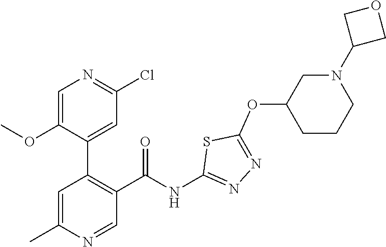 [SMILES: COc1cnc(Cl)cc1-c1cc(C)ncc1C(=O)Nc1nnc(OC2CCCN(C3COC3)C2)s1] 2′-chloro-5′-methoxy-6-methyl-N- (5-((1-(oxetan-3-yl)piperidin-3- yl)oxy)-1,3,4-thiadiazol-2-yl)- [4,4′-bipyridine]-3-carboxamide | ¹H NMR (400 MHz, CHLOROFORM-d) δ 8.98 (s, 1H), 8.04 (s, 1H), 7.27 (s, 1H), 7.19 (s, 1H), 5.16- 5.02 (m, 1H), 4.71- 4.58 (m, 4H), 3.75 (s, 3H), 3.69-3.54 (m, 1H), 2.92-2.81 (m, 1H), 2.73 (s, 3H), 2.55-2.40 (m, 2H), 2.32-2.21 (m, 1H), 2.13-2.04 (m, 1H), 1.97-1.87 (m, 1H), 1.78-1.65 (m, 2H) | MS (ESI) calc′d for (C₂₃H₂₅ClN₆O₄S) (M + 1)⁺, 517.0, found 517.2 |
| Example 251 | 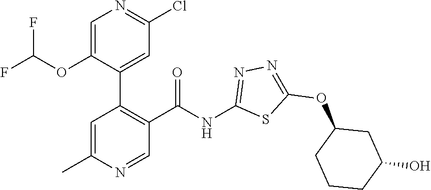 [SMILES: Cc1cc(-c2cc(Cl)ncc2OC(F)F)c(C(=O)Nc2nnc(O[C@@H]3CCC[C@@H](O)C3)s2)cn1] 2′-chloro-5′-(difluoromethoxy)-N- (5-(((1R,3R)-3- hydroxycyclohexyl)oxy)-1,3,4- thiadiazol-2-yl)-6-methyl-[4,4′- bipyridine]-3-carboxamide. The absolute stereochemistry was not determined and the relative stereochemistry was determined by X-ray crystallography. | 1H-NMR (400 MHz, DMSO-d⁶): δ 13.01 (s, 1H), 8.96 (s, 1H), 8.33 (s, 1H), 7.72 (s, 1H), 7.45 (s, 1H), 7.14 (t, J = 72.4 Hz, 1H), 5.22-5.15 (m, 1H), 4.63 (d, J = 4.0 Hz, 1H), 3.91-3.82 (m, 1H), 2.61 (s, 3H), 1.96-1.88 (m, 1H), 1.87-1.75 (m, 2H), 1.74-1.50 (m, 4H), 1.45-1.34 (m, 1H). | MS (ESI) calculated for (C₂₁H₂₀ClF₂N₅O₄S) (M + 1)⁺, 512.10; found, 512.0 |
| Example 252 | 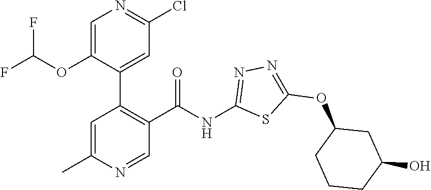 [SMILES: Cc1cc(-c2cc(Cl)ncc2OC(F)F)c(C(=O)Nc2nnc(O[C@@H]3CCC[C@H](O)C3)s2)cn1] 2′-chloro-5′-(difluoromethoxy)-N- (5-(((1R,3S)-3- hydroxycyclohexyl)oxy)-1,3,4- thiadiazol-2-yl)-6-methyl-[4,4′- bipyridine]-3-carboxamide | 1H-NMR (400 MHz, DMSO-d⁶): δ 9.03 (s, 1H), 8.30 (s, 1H), 7.61 (s, 1H), 7.31 (s, 1H), 7.12 (t, J = 73.2 Hz, 1H), 4.81-4.69 (m, 2H), 3.55-3.45 (m, 1H), 2.59 (s, 3H), 2.41-2.35 (m, 1H), 2.12-2.05 (m, 1H), 1.82-1.65 (m, 2H), 1.32-1.20 (m, 3H), 1.13-1.00 (m, 1H). | MS (ESI) calculated for (C₂₁H₂₀ClF₂N₅O₄S) (M + 1)⁺, 512.0; found, 512.0 |
| Example 253 | 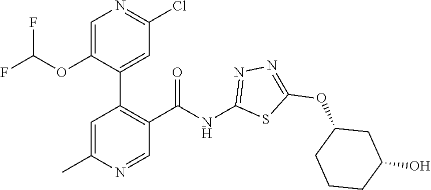 [SMILES: Cc1cc(-c2cc(Cl)ncc2OC(F)F)c(C(=O)Nc2nnc(O[C@H]3CCC[C@@H](O)C3)s2)cn1] 2′-chloro-5′-(difluoromethoxy)-N- (5-(((1S,3R)-3- hydroxycyclohexyl)oxy)-1,3,4- thiadiazol-2-yl)-6-methyl-[4,4′- bipyridine]-3-carboxamide | 1H-NMR (400 MHz, DMSO-d⁶): δ 9.02 (s, 1H), 8.30 (s, 1H), 7.62 (s, 1H), 7.32 (s, 1H), 7.12 (t, J = 72.8 Hz, 1H), 4.81-4.69 (m, 2H), 3.55-3.45 (m, 1H), 2.59 (s, 3H), 2.41-2.35 (m, 1H), 2.12-2.05 (m, 1H), 1.82-1.66 (m, 2H), 1.32-1.20 (m, 3H), 1.13-1.00 (m, 1H). | MS (ESI) calculated for (C₂₁H₂₀ClF₂N₅O₄S) (M + 1)⁺, 512.0; found, 512.0, |
| Example 254 | 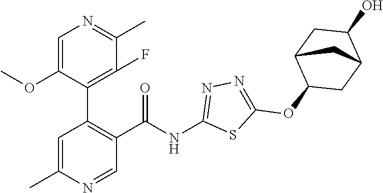 [SMILES: COc1cnc(C)c(F)c1-c1cc(C)ncc1C(=O)Nc1nnc(O[C@@H]2C[C@@H]3C[C@H]2C[C@H]3O)s1] 3′-fluoro-N-(5-(((1S,2R,4S,5R)-5- hydroxybicyclo[2.2.1]heptan-2- yl)oxy)-1,3,4-thiadiazol-2-yl)-5′- methoxy-2′,6-dimethyl-[4,4′- bipyridine]-3-carboxamide. The stereochemistry was not determined. | 1H-NMR (400 MHz, DMSO-d₆): δ 12.97 (brs, 1H), 8.91 (s, 1H), 8.17 (s, 1H), 7.38 (s, 1H), 4.67- 4.50 (m, 2H), 3.68 (d, J = 1.20 Hz, 3H), 3.58-3.53 (m, 1H), 2.56 (s, 3H), 2.46- 2.42 (m, 1H), 2.42 (d, J = 2.8 Hz, 3H), 2.10 (d, J = 4.4 Hz, 1H), 1.67-1.59 (m, 2H), 1.57-1.49 (m, 1H), 1.48-1.40 (m, 2H), 1.24-1.19 (m, 1H). | MS (ESI) calculated for (C₂₃H₂₄FN₅O₄S) (M + 1)⁺ 486.16; found, 486.2 |
| Example 255 | 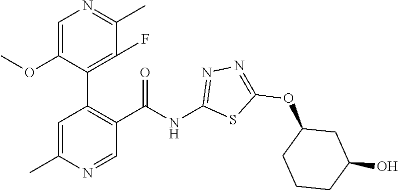 [SMILES: COc1cnc(C)c(F)c1-c1cc(C)ncc1C(=O)Nc1nnc(O[C@@H]2CCC[C@H](O)C2)s1] 3′-fluoro-N-(5-(((1R,3S)-3- hydroxycyclohexyl)oxy)-1,3,4- thiadiazol-2-yl)-5′-methoxy-2′,6- dimethyl-[4,4′-bipyridine]-3- carboxamide. The absolute stereochemistry was not determined and the relative stereochemistry was determined by X-ray crystallography. | 1H-NMR (400 MHz, DMSO-d6): δ 12.94 (s, 1H), 8.90 (s, 1H), 8.18 (s, 1H), 7.39 (s, 1H), 4.88-4.75 (m, 2H), 3.69 (s, 3H), 3.55-3.45 (m, 1H), 2.59 (s, 3H), 2.43 (d, J = 3.20 Hz, 3H), 2.42-2.37 (m, 1H), 2.15-2.07 (m, 1H), 1.83-1.67 (m, 2H), 1.35-1.20 (m, 3H), 1.15-1.00 (m, 1H). | MS (ESI) calculated for (C₂₂H₂₄FN₅O₄S) (M)⁺, 474.16; found, 474.0. |
| Example 256 | 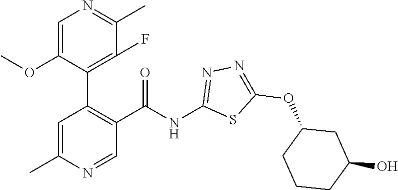 [SMILES: COc1cnc(C)c(F)c1-c1cc(C)ncc1C(=O)Nc1nnc(O[C@H]2CCC[C@H](O)C2)s1] 3′-fluoro-N-(5-(((1S,3S)-3- hydroxycyclohexyl)oxy)-1,3,4- thiadiazol-2-yl)-5′-methoxy-2′,6- dimethyl-[4,4′-bipyridine]-3- carboxamide. The absolute stereochemistry was not determined and the relative stereochemistry was determined by X-ray crystallography. | 1H-NMR (400 MHz, DMSO-d⁶): δ 12.95 (s, 1H), 8.90 (s, 1H), 8.18 (s, 1H), 7.39 (s, 1H), 5.21-5.13 (m, 1H), 4.63 (d, J = 4.00 Hz, 1H), 3.92-3.83 (m, 1H), 3.69 (s, 3H), 2.58 (s, 3H), 2.42 (d, J = 3.2 Hz, 3H), 1.92- 1.88 (m, 1H), 1.87- 1.75 (m, 2H), 1.72- 1.49 (m, 4H), 1.42- 1.32 (m, 1H). | MS (ESI) calculated for (C₂₂H₂₄FN₅O₄S) (M + 1)⁺, 474.16; found, 474.0. |
| Example 257 | 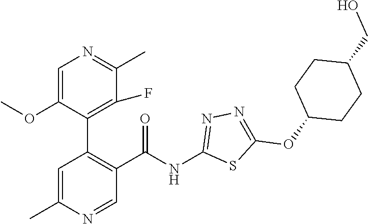 [SMILES: COc1cnc(C)c(F)c1-c1cc(C)ncc1C(=O)Nc1nnc(O[C@H]2CC[C@@H](CO)CC2)s1] 3′-fluoro-N-(5-(((1s,4s)-4- (hydroxymethyl)cyclohexyl)oxy)- 1,3,4-thiadiazol-2-yl)-5′-methoxy- 2′,6-dimethyl-[4,4′-bipyridine]-3- carboxamide . The relative stereochemistry was determined as synthesis used starting material with known relative stereochemistry. | 1H-NMR (400 MHz, DMSO-d⁶): δ 12.95 (s, 1H), 8.89 (s, 1H), 8.18 (s, 1H), 7.40 (s, 1H), 5.08 (s, 1H), 4.44 (t, J = 5.60 Hz, 1H), 3.69 (s, 3H), 3.26 (t, J = 6.00 Hz, 2H), 2.59 (s, 3H), 2.43 (d, J = 3.20 Hz, 3H), 2.07- 1.96 (m, 2H), 1.70- 1.40 (m, 5H), 1.38- 1.20 (m, 2H). | MS (ESI) calculated for (C₂₃H₂₆FN₅O₄S) (M + 1)⁺, 488.18; found, 488.0. |
| Example 258 | 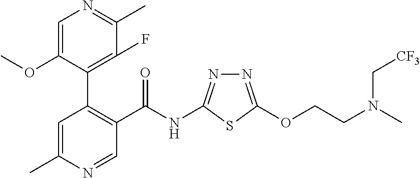 [SMILES: COc1cnc(C)c(F)c1-c1cc(C)ncc1C(=O)Nc1nnc(OCCN(C)CC(F)(F)F)s1] 3′-fluoro-5′-methoxy-2′,6- dimethyl- N-(5-(2-(methyl(2,2,2- trifluoroethyl) amino)ethoxy)-1,3,4-thiadiazol-2- yl)-[4,4′-bipyridine]-3- carboxamide | ¹H NMR (400 MHz, DMSO-d₆) δ 12.97 (br s, 1H), 8.97-8.75 (m, 1H), 8.17 (s, 1H), 7.39 (s, 1H), 4.49 (t, J = 5.4 Hz, 2H), 3.67 (s, 3H), 3.33-3.24 (m, 2H), 2.98 (t, J = 5.6 Hz, 2H), 2.58 (s, 3H), 2.44 (s, 3H), 2.42 (d, J = 3.4 Hz, 3H) | MS (ESI) calc′d for (C₂₁H₂₂F4N₆O₃S) (M + 1)⁺, 515.1, found 515.1. |
| Example 260 | 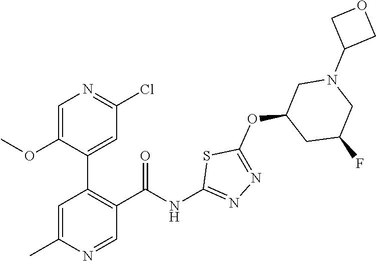 [SMILES: COc1cnc(Cl)cc1-c1cc(C)ncc1C(=O)Nc1nnc(O[C@@H]2C[C@H](F)CN(C3COC3)C2)s1] 2′-chloro-N-(5-(((3R,5S)-5-fluoro- 1-(oxetan-3-yl)piperidin-3- yl)oxy)-1,3,4-thiadiazol-2-yl)-5′- methoxy-6-methyl-[4,4′- bipyridine]-3-carboxamide | ¹H NMR (400 MHz, METHANOL-d₄) δ 8.80 (s, 1H), 8.10 (s, 1H), 7.50 (s, 1H), 7.45 (s, 1H), 5.17- 5.05 (m, 1H), 4.86- 4.79 (m, 1H), 4.70 (td, J = 6.7, 1.7 Hz, 2H), 4.62 (dt, J = 12.7, 6.4 Hz, 2H), 3.74 (s, 3H), 3.72- 3.65 (m, 1H), 2.93- 2.84 (m, 1H), 2.82- 2.71 (m, 1H), 2.69 (s, 3H), 2.60-2.40 (m, 3H), 2.15-2.02 (m, 1H | MS (ESI) calc′d for (C₂₃H₂₄ClFN₆O₄S) (M + 1)⁺, 535.0, found 535.3 |
| Example 261 | 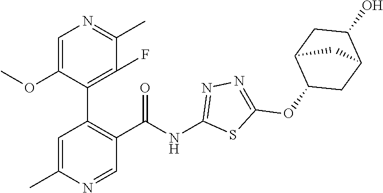 [SMILES: COc1cnc(C)c(F)c1-c1cc(C)ncc1C(=O)Nc1nnc(O[C@H]2C[C@H]3C[C@@H]2C[C@@H]3O)s1] 3′-fluoro-N-(5-(((1R,2S,4R,5S)-5- hydroxybicyclo[2.2.1]heptan-2- yl)oxy)-1,3,4-thiadiazol-2-yl)-5′- methoxy-2′,6-dimethyl-[4,4′- bipyridine]-3-carboxamide. The stereochemistry was not determined. | 1H-NMR (400 MHz, DMSO-d₆): δ 12.97 (brs, 1H), 8.94 (s, 1H), 8.16 (s, 1H), 7.33 (s, 1H), 4.67- 4.49 (m, 2H), 3.68 (d, J = 0.8 Hz, 3H), 3.58- 3.53 (m, 1H), 2.57 (s, 3H), 2.46-2.42 (m, 1H), 2.42 (d, J = 2.8 Hz, 3H), 2.09 (d, J = 4.4 Hz, 1H), 1.67- 1.59 (m, 2H), 1.56- 1.49 (m, 1H), 1.48- 1.40 (m, 2H), 1.24- 1.19 (m, 1H). | MS (ESI) calculated for (C₂₃H₂₄FN₅O₄S) (M + 1)⁺ 486.16; found, 486.2 |
| Example 263 | 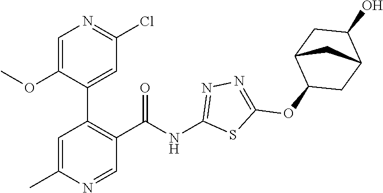 [SMILES: COc1cnc(Cl)cc1-c1cc(C)ncc1C(=O)Nc1nnc(O[C@@H]2C[C@@H]3C[C@H]2C[C@H]3O)s1] 2′-chloro-N-(5-(((1S,2R,4S,5R)-5- hydroxybicyclo[2.2.1]heptan-2- yl)oxy)-1,3,4-thiadiazol-2-yl)-5′- methoxy-6-methyl-[4,4′- bipyridine]-3-carboxamide The stereochemistry was not determined. | 1H-NMR (400 MHz, DMSO-d₆): δ 12.88 (s, 1H), 8.81 (s, 1H), 8.17 (s, 1H), 7.52 (s, 1H), 7.42 (s, 1H), 4.69-4.61 (m, 2H), 3.63 (s, 3H), 3.59- 3.54 (m, 1H), 2.59 (s, 3H), 2.49-2.46 (m, 1H), 2.13-2.08 (m, 1H), 1.70-1.59 (m, 2H), 1.58-1.50 (m, 1H), 1.49-1.40 (m, 2H), 1.27-1.17 (m, 1H). | MS (ESI) calculated for (C₂₂H₂₂ClN₅O₄S) (M + 1)⁺ 488.12; found, 488.0 |
| Example 264 | 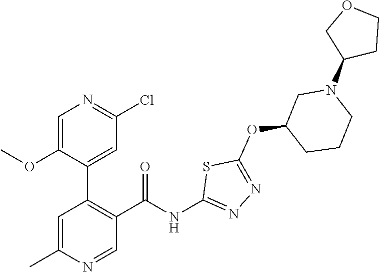 [SMILES: COc1cnc(Cl)cc1-c1cc(C)ncc1C(=O)Nc1nnc(O[C@@H]2CCCN([C@@H]3CCOC3)C2)s1] 2′-chloro-5′-methoxy-6-methyl-N- (5-(((R)-1-((R)-tetrahydrofuran-3- yl)piperidin-3-yl)oxy)-1,3,4- thiadiazol-2-yl)-[4,4′-bipyridine]- 3-carboxamide. The stereochemistry determined using VCD. | ¹H NMR (400 MHz, CHLOROFORM-d) δ 8.97 (s, 1H), 8.04 (s, 1H), 7.28 (s, 1H), 7.18 (s, 1H), 5.09- 4.99 (m, 1H), 3.96- 3.89 (m, 1H), 3.84 (dd, J = 8.8, 6.8 Hz, 1H), 3.80-3.76 (m, 1H), 3.74 (s, 3H), 3.65 (dd, J = 8.8, 6.4 Hz, 1H), 3.13-3.04 (m, 1H), 3.02-2.95 (m, 1H), 2.72 (s, 3H), 2.55-2.35 (m, 3H), 2.09-1.98 (m, 2H), 1.92-1.80 (m, 2H), 1.76-1.59 (m, 2H) | MS (ESI) calc′d for (C₂₄H₂₇ClN₆O₄S) (M + 1)⁺, 531.0, found 531.1 |
| Example 265 | 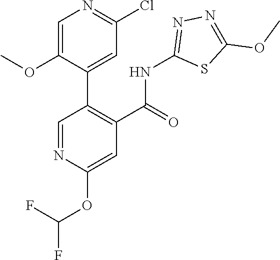 2′-chloro-6-(difluoromethoxy)-5′- methoxy-N-(5-methoxy-1,3,4- thiadiazol-2-yl)-[3,4′-bipyridine]- 4-carboxamide | 1H-NMR (400 MHz, DMSO-d⁶): δ 13.10 (s, 1H), 8.42 (s, 1H), 8.15 (s, 1H), 7.83 (t, J = 72 Hz, 1H), 7.59 (s, 1H), 7.51 (s, 1H), 4.09 (s, 3H), 3.62 (s, 3H) | MS (ESI) calculated for (C₁₆H₁₂ClF₂N₅O₄S) (M + 1)⁺, 444.04; found, 444.0. |
| Example 266 | 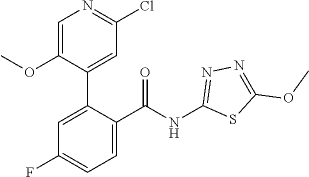 2-(2-chloro-5-methoxypyridin-4- yl)-4-fluoro-N-(5-methoxy-1,3,4- thiadiazol-2-yl)benzamide | ¹H NMR (DMSO-d₆, 400 MHz) δ 12.74 (br s, 1H), 8.12 (s, 1H), 7.84 (dd, 1H, J = 5.6, 8.6 Hz), 7.50 (s, 1H), 7.4-7.5 (m, 2H), 4.06 (s, 3H), 3.61 (s, 3H) | MS (ESI) calc′d for (C₁₆H₁₂ClFN₄O₃S) (M + 1)⁺, 395.0, found 395.2. |
| Example 267 | 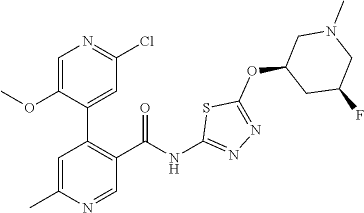 [SMILES: COc1cnc(Cl)cc1-c1cc(C)ncc1C(=O)Nc1nnc(O[C@@H]2C[C@H](F)CN(C)C2)s1] 2′-chloro-N-(5-(((3R,5S)-5-fluoro- 1-methylpiperidin-3-yl)oxy)- 1,3,4-thiadiazol-2-yl)-5′-methoxy- 6-methyl-[4,4′-bipyridine]-3- carboxamide | ¹H NMR (400 MHz, METHANOL-d₄) δ 8.80 (s, 1H), 8.10 (s, 1H), 7.53-7.49 (m, 1H), 7.48-7.38 (m, 1H), 5.21-5.11 (m, 1H), 4.85-4.70 (m, 1H), 3.74 (s, 3H), 2.88- 2.81 (m, 1H), 2.81- 2.71 (m, 2H), 2.69 (s, 3H), 2.67-2.58 (m, 1H), 2.36 (s, 3H), 2.34- 2.17 (m, 2H) | MS (ESI) calc′d for (C₂₁H₂₂ClFN₆O₃S) (M + 1)⁺, 493.0, found 493.3 |
| Example 268 | 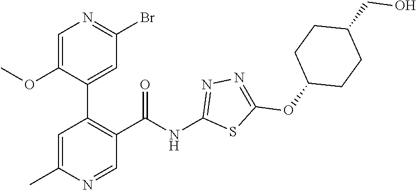 [SMILES: COc1cnc(Br)cc1-c1cc(C)ncc1C(=O)Nc1nnc(O[C@H]2CC[C@@H](CO)CC2)s1] 2′-bromo-N-(5-(((1s,4s)-4- (hydroxymethyl)cyclohexyl)oxy)- 1,3,4-thiadiazol-2-yl)-5′-methoxy- 6-methyl-[4,4′-bipyridine]-3- carboxamide. | 1H-NMR (400 MHz, DMSO-d⁶): δ 12.88 (s, 1H), 8.84 (s, 1H), 8.16 (s, 1H), 7.58 (s, 1H), 7.36 (s, 1H), 5.05 (s, 1H), 4.45-4.38 (m, 1H), 3.63 (s, 3H), 3.30-3.20 (m, 2H), 2.57 (s, 3H), 2.08- 1.98 (m, 2H), 1.70- 1.42 (m, 5H), 1.32- 1.20 (m, 2H). | MS (ESI) calculated for (C₂₂H₂₄BrN₅O₄S) (M + 1)⁺, 534.08; found, 534.0. |
| Example 269 | 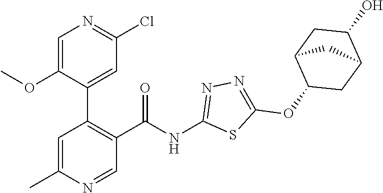 [SMILES: COc1cnc(Cl)cc1-c1cc(C)ncc1C(=O)Nc1nnc(O[C@H]2C[C@H]3C[C@@H]2C[C@@H]3O)s1] 2′-chloro-N-(5-(((1R,2S,4R,5S)-5- hydroxybicyclo[2.2.1]heptan-2- yl)oxy)-1,3,4-thiadiazol-2-yl)-5′- methoxy-6-methyl-[4,4′- bipyridine]-3-carboxamide. The stereochemistry was not determined. | 1H-NMR (400 MHz, DMSO-d₆): δ 12.88 (s, 1H), 8.81 (s, 1H), 8.17 (s, 1H), 7.52 (s, 1H), 7.41 (s, 1H), 4.68-4.61 (m, 2H), 3.63 (s, 3H), 3.59- 3.54 (m, 1H), 2.59 (s, 3H), 2.49-2.46 (m, 1H), 2.13-2.08 (m, 1H), 1.70-1.59 (m, 2H), 1.58-1.50 (m, 1H), 1.48-1.40 (m, 2H), 1.27-1.17 (m, 1H). | MS (ESI) calculated for (C₂₂H₂₂ClN₅O₄S) (M + 1)⁺ 488.12; found, 488.0 |
| Example 271 | 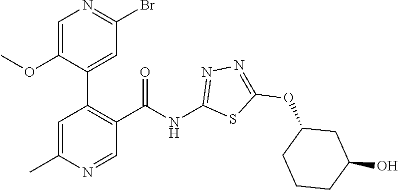 [SMILES: COc1cnc(Br)cc1-c1cc(C)ncc1C(=O)Nc1nnc(O[C@H]2CCC[C@H](O)C2)s1] 2′-bromo-N-(5-(((1S,3S)-3- hydroxycyclohexyl)oxy)-1,3,4- thiadiazol-2-yl)-5′-methoxy-6- methyl-[4,4′-bipyridine]-3- carboxamide. The stereochemistry was determined by X-ray crystallography. | 1H-NMR (400 MHz, DMSO-d⁶): δ 12.88 (s, 1H), 8.81 (s, 1H), 8.17 (s, 1H), 7.62 (s, 1H), 7.40 (s, 1H), 5.22-5.15 (m, 1H), 4.63 (d, J = 4.00 Hz, 1H), 3.91-3.83 (m, 1H), 3.63 (s, 3H), 2.58 (s, 3H), 1.95-1.89 (m, 1H), 1.88-1.75 (m, 2H), 1.74-1.50 (m, 4H), 1.42-1.35 (m, 1H). | MS (ESI) calculated for (C₂₁H₂₂BrN₅O₄S) (M)⁺, 520.4; found, 519.8. |
| Example 273 | 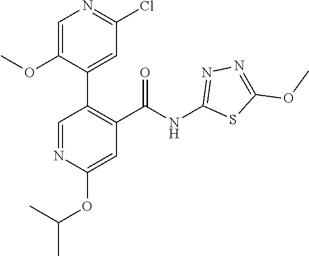 2′-chloro-6-isopropoxy-5′- methoxy-N-(5-methoxy-1,3,4- thiadiazol-2-yl)-[3,4′-bipyridine]- 4-carboxamide | 1H-NMR (400 MHz, DMSO-d⁶): δ 12.92 (s, 1H), 8.27 (s, 1H), 8.10 (s, 1H), 7.53 (s, 1H), 7.10 (s, 1H), 5.37-5.31 (m, 1H), 4.08 (s, 3H), 3.59 (s, 3H), 1.36 (d, J = 6.40 Hz, 6H). | MS (ESI) calculated for (C₁₈H₁₈ClN₅O₄S) (M + 1)⁺, 436.08; found, 435.8. |
| Example 274 | 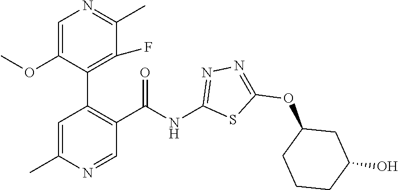 [SMILES: COc1cnc(C)c(F)c1-c1cc(C)ncc1C(=O)Nc1nnc(O[C@@H]2CCC[C@@H](O)C2)s1] 3′-fluoro-N-(5-(((1R,3R)-3- hydroxycyclohexyl)oxy)-1,3,4- thiadiazol-2-yl)-5′-methoxy-2′,6- dimethyl-[4,4′-bipyridine]-3- carboxamide. The absolute stereochemistry was not determined and the relative stereochemistry was determined by X-ray crystallography. | 1H-NMR (400 MHz, DMSO-d⁶): δ 12.95 (s, 1H), 8.94 (s, 1H), 8.17 (s, 1H), 7.34 (s, 1H), 5.21-5.11 (m, 1H), 4.62 (d, J = 3.60 Hz, 1H), 3.91-3.83 (m, 1H), 3.69 (s, 3H), 2.58 (s, 3H), 2.42 (d, J = 3.2 Hz, 3H), 1.95- 1.88 (m, 1H), 1.87- 1.73 (m, 2H), 1.72- 1.49 (m, 4H), 1.44- 1.32 (m, 1H). | MS (ESI) calculated for (C₂₂H₂₄FN₅O₄S) (M + 1)⁺, 474.16; found, 474.0. |
| Example 275 | 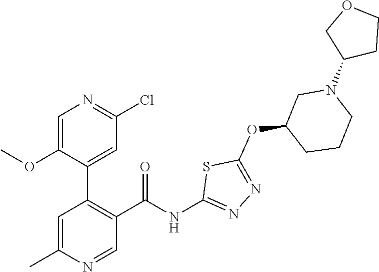 [SMILES: COc1cnc(Cl)cc1-c1cc(C)ncc1C(=O)Nc1nnc(O[C@@H]2CCCN([C@H]3CCOC3)C2)s1] 2′-chloro-5′-methoxy-6-methyl-N- (5-(((R)-1-((S)-tetrahydrofuran-3- yl)piperidin-3-yl)oxy)-1,3,4- thiadiazol-2-yl)-[4,4′-bipyridine]- 3-carboxamide. The stereochemistry was dertermined using VCD. | ¹H NMR (400 MHz, CHLOROFORM-d) δ 8.97 (s, 1H), 8.04 (s, 1H), 7.28 (s, 1H), 7.18 (s, 1H), 5.12-4.98 (m, 1H), 3.94 (td, J = 8.7, 4.6 Hz, 1H), 3.85 (dd, J = 8.8, 6.8 Hz, 1H), 3.80-3.76 (m, 1H), 3.75 (s, 3H), 3.67 (dd, J = 8.8, 6.4 Hz, 1H), 3.14-3.05 (m, 1H), 2.81 (br dd, J = 11.2, 2.9 Hz, 1H), 2.72 (s, 3H), 2.67- 2.57 (m, 2H), 2.40- 2.30 (m, 1H), 2.09- 1.98 (m, 2H), 1.92- 1.80 (m, 2H), 1.78- 1.58 (m, 2H) | MS (ESI) calc′d for (C₂₄H₂₇ClN₆O₄S) (M + 1)⁺, 531.0, found 531.1 |
| Example 278 | 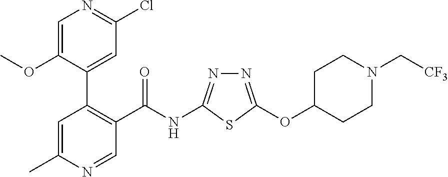 [SMILES: COc1cnc(Cl)cc1-c1cc(C)ncc1C(=O)Nc1nnc(OC2CCN(CC(F)(F)F)CC2)s1] 2′-chloro-5′-methoxy-6-methyl- N-(5-((1-(2,2,2- trifluoroethyl)piperidin-4-yl)oxy)- 1,3,4-thiadiazol-2-yl)-[4,4′- bipyridine]-3-carboxamide | ¹H NMR (400 MHz, DMSO-d₆) δ 12.89 (br s, 1H), 8.85-8.77 (m, 1H), 8.17 (s, 1H), 7.53 (s, 1H), 7.43 (s, 1H), 4.09 (q, J = 5.2 Hz, 1H), 3.61 (s, 3H), 3.24-3.18 (m, 2H), 2.89-2.78 (m, 2H), 2.61 (br d, J = 2.9 Hz, 1H), 2.58 (s, 3H), 2.56 (br d, J = 3.4 Hz, 1H), 2.09-1.97 (m, 2H), 1.83-1.70 (m, 2H) | MS (ESI) calc′d for (C₂₂H₂₂ClF₃N₆O₃S) (M + 1)⁺, 543.1, found 543.0. |
| Example 279 | 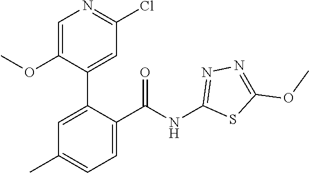 2-(2-chloro-5-methoxypyridin-4- yl)-N-(5-methoxy-1,3,4- thiadiazol-2-yl)-4- methylbenzamide | ¹H NMR (DMSO-d₆, 400 MHz) δ 12.72 (br s, 1H), 8.17 (s, 1H), 7.75 .(d, 1H, J = 7.8 Hz), 7.52 (s, 1H), 7.48 (dd, 1H, J = 2.0, 8.3 Hz), 7.38 (s, 1H), 4.15 (s, 3H), 3.68 (s, 3H), 2.50 (s, 3H) | MS (ESI) calc′d for (C₁₇H₁₅ClN₄O₃S) (M + 1)⁺, 391.1, found 391.1 |
| Example 280 | 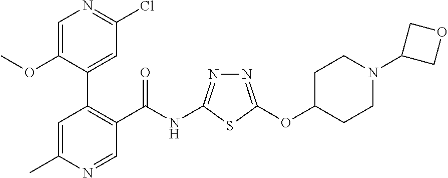 [SMILES: COc1cnc(Cl)cc1-c1cc(C)ncc1C(=O)Nc1nnc(OC2CCN(C3COC3)CC2)s1] 2′-chloro-5′-methoxy-6-methyl-N- (5-((1-(oxetan-3-yl)piperidin-4- yl)oxy)-1,3,4-thiadiazol-2-yl)- [4,4′-bipyridine]-3-carboxamide | ¹H NMR (400 MHz, METHANOL-d₄) δ 8.82-8.78 (m, 1H), 8.10 (s, 1H), 7.52- 7.49 (m, 1H), 7.45 (s, 1H), 5.13 (br s, 1H), 4.81-4.74 (m, 2H), 4.68 (t, J = 6.4 Hz, 2H), 3.89-3.79 (m, 1H), 3.74 (s, 3H), 2.84 (br d, J = 5.9 Hz, 2H), 2.69 (s, 3H), 2.66-2.57 (m, 2H), 2.28-2.17 (m, 2H), 2.13-2.02 (m, 2H) | MS (ESI) calc′d for (C₂₃H₂₅ClN₆O₄S) (M + 1)⁺, 517.1, found 517.0. |
| Example 281 | 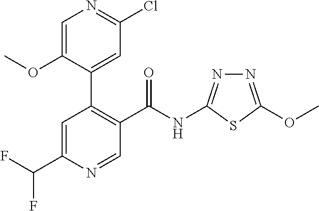 2′-chloro-6-(difluoromethyl)-5′- methoxy-N-(5-methoxy-1,3,4- thiadiazol-2-yl)-[4,4′-bipyridine]- 3-carboxamide | ¹H NMR (400 MHz, DMSO-d₆) δ 13.12 (br s, 1H), 9.06 (s, 1H), 8.22 (s, 1H), 7.87 (s, 1H), 7.66 (s, 1H), 7.10 (t, J = 54.5 Hz, 1H), 4.09 (s, 3H), 3.65 (s, 3H). | MS (ESI) calc′d for (C₁₆H₁₂ClF₂N₅O₃S) (M + 1)⁺, 428.0; found 428.1 |
| Example 282 | 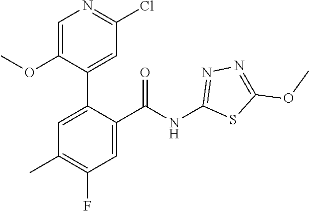 2-(2-chloro-5-methoxypyridin-4- yl)-5-fluoro-N-(5-methoxy-1,3,4- thiadiazol-2-yl)-4-methyl benzamide | ¹H NMR (DMSO-d₆, 400 MHz) δ 12.74 (br s, 1H), 8.09 (s, 1H), 7.60 (d, 1H, J = 9.8 Hz), 7.4-7.5 (m, 2H), 4.07 (s, 3H), 3.59 (s, 3H), 2.33 (s, 3H) | MS (ESI) calc′d for (C₁₇H₁₄ClFN₄O₃S) (M + 1)⁺, 409.0, found 409.1 |
| Example 283 | 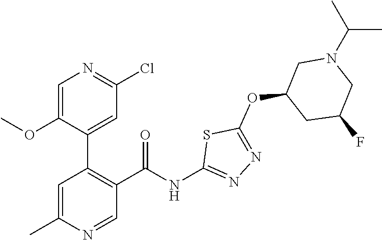 [SMILES: COc1cnc(Cl)cc1-c1cc(C)ncc1C(=O)Nc1nnc(O[C@@H]2C[C@H](F)CN(C(C)C)C2)s1] 2′-chloro-N-(5-(((3R,5S)-5-fluoro- 1-isopropylpiperidin-3-yl)oxy)- 1,3,4-thiadiazol-2-yl)-5′-methoxy- 6-methyl-[4,4′-bipyridine]-3- carboxamide | ¹H NMR (400 MHz, METHANOL-d₄) δ 8.80 (s, 1H), 8.10 (s, 1H), 7.55-7.47 (m, 1H), 7.45 (s, 1H), 5.16-5.01 (m, 1H), 4.80-4.65 (m, 1H), 3.74 (s, 3H), 3.14 (br d, J = 11.7 Hz, 1H), 3.07-2.91 (m, 2H), 2.71-2.62 (m, 4H), 2.58-2.40 (m, 2H), 2.06-1.88 (m, 1H), 1.11 (d, J = 6.4 Hz, 6H) | MS (ESI) calc′d for (C₂₃H₂₆ClFN₆O₃S) (M + 1)⁺, 521.0, found 521.3 |
| Example 284 | 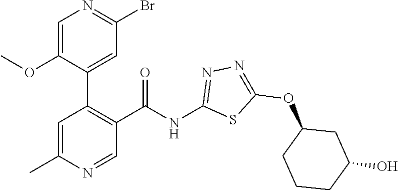 [SMILES: COc1cnc(Br)cc1-c1cc(C)ncc1C(=O)Nc1nnc(O[C@@H]2CCC[C@@H](O)C2)s1] 2′-bromo-N-(5-(((1R,3R)-3- hydroxycyclohexyl)oxy)-1,3,4- thiadiazol-2-yl)-5′-methoxy-6- methyl-[4,4′-bipyridine]-3- carboxamide. The stereochemistry was determined by X-ray crystallography. | 1H-NMR (400 MHz, DMSO-d⁶): δ 12.87 (s, 1H), 8.80 (s, 1H), 8.18 (s, 1H), 7.64 (s, 1H), 7.43 (s, 1H), 5.24-5.15 (m, 1H), 4.63 (d, J = 4.00 Hz, 1H), 3.91-3.83 (m, 1H), 3.63 (s, 3H), 2.59 (s, 3H), 1.95-1.89 (m, 1H), 1.87-1.75 (m, 2H), 1.74-1.50 (m, 4H), 1.42-1.35 (m, 1H). | MS (ESI) calculated for (C₂₁H₂₂BrN₅O₄S) (M)⁺, 520.4; found, 520.0. |
| Example 285 | 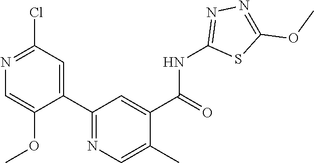 [SMILES: COc1nnc(NC(=O)c2cc(-c3cc(Cl)ncc3OC)ncc2C)s1] 2′-chloro-5′-methoxy-N-(5- methoxy-1,3,4-thiadiazol-2-yl)-5- methyl-[2,4′-bipyridine]-4- carboxamide. | 1H-NMR (400 MHz, DMSO-d6): δ 13.07 (s, 1H), 8.74 (s, 1H), 8.39 (s, 1H), 8.14 (s, 1H), 7.85 (s, 1H), 4.11 (s, 3H), 3.99 (s, 3H), 2.44 (s, 3H). | MS (ESI) calculated for (C₁₆H₁₄ClN₅O₃S) (M + 1)⁺, 392.06; found, 392.0. |
| Example 286 | 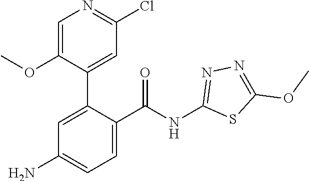 4-amino-2-(2-chloro-5- methoxypyridin-4-yl)-N-(5- methoxy-1,3,4-thiadiazol-2- yl)benzamide | ¹H NMR (METHANOL-d₄, 400 MHz) δ 7.96 (s, 1H), 7.53 (d, 1H, J = 8.8 Hz), 7.34 (s, 1H), 6.75 (dd, 1H, J = 2.2, 8.6 Hz), 6.60 (d, 1H, J = 2.4 Hz), 4.10 (s, 3H), 3.69 (s, 3H) | MS (ESI) calc′d for (C₁₆H₁₄ClN₅O₃S) (M + 1)⁺, 392.1, found 392.1. |
| Example 287 | 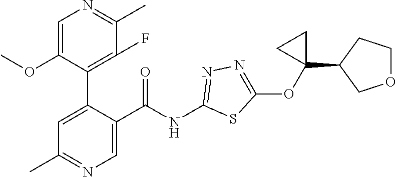 [SMILES: COc1cnc(C)c(F)c1-c1cc(C)ncc1C(=O)Nc1nnc(OC2([C@H]3CCOC3)CC2)s1] 3′-fluoro-5′-methoxy-2′,6- dimethyl-N-(5-(1-((S)- tetrahydrofuran-3- yl)cyclopropoxy)-1,3,4-thiadiazol- 2-yl)-[4,4′-bipyridine]-3- carboxamide. The stereochemistry was not determined. | 1H-NMR (400 MHz, DMSO-d⁶): δ 13.03 (s, 1H), 8.90 (s, 1H), 8.19 (s, 1H), 7.40 (s, 1H), 3.79-3.71 (m, 2H), 3.69 (s, 3H), 3.62 (q, J = 7.60 Hz, 1H), 3.42-3.36 (m, 1H), 3.10 (t, J = 7.60 Hz, 1H), 2.59 (s, 3H), 2.43 (d, J = 2.80 Hz, 3H), 2.00-1.90 (m, 1H), 1.61-1.50 (m, 1H), 1.10-1.02 (m, 2H), 0.98-0.90 (m, 2H), | MS (ESI) calculated for (C₂₃H₂₄FN₅O₄S) (M + 1)⁺, 486.16; found, 486.0. |
| Example 288 | 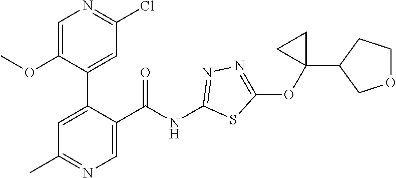 [SMILES: COc1cnc(Cl)cc1-c1cc(C)ncc1C(=O)Nc1nnc(OC2(C3CCOC3)CC2)s1] 2′-chloro-5′-methoxy-6-methyl-N- (5-(1-(tetrahydrofuran-3- yl)cyclopropoxy)-1,3,4-thiadiazol- 2-yl)-[4,4′-bipyridine]-3- carboxamide | 1H-NMR (400 MHz, DMSO-d⁶): δ 12.98 (brs, 1H), 8.82 (s, 1H), 8.18 (s, 1H), 7.53 (s, 1H), 7.44 (s, 1H), 3.80-3.70 (m, 2H), 3.68-3.59 (m, 4H), 3.42-3.38 (m, 1H), 3.15-3.05 (m, 1H), 2.59 (s, 3H), 2.01- 1.90 (m, 1H), 1.61- 1.51 (m, 1H), 1.12- 1.05 (m, 2H), 0.98- 0.89 (m, 2H). | MS (ESI) calculated for (C₂₂H₂₂ClN₅O₄S) (M + 1)⁺, 488.12; found, 488.0. |
| Example 289 | 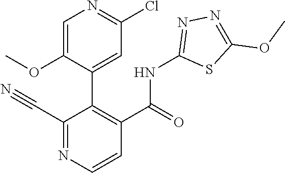 2′-chloro-2-cyano-5′-methoxy-N- (5-methoxy-1,3,4-thiadiazol-2-yl)- [3,4′-bipyridine]-4-carboxamide. | 1H-NMR (400 MHz, DMSO-d⁶): δ 13.39 (brs, 1H), 9.01 (d, J = 4.80 Hz, 1H), 8.32 (s, 1H), 8.18 (d, J = 5.20 Hz, 1H), 7.75 (s, 1H), 4.07 (s, 3H), 3.73 (s, 3H). | MS (ESI) calculated for (C₁₆H₁₁ClN₆O₃S) (M + 1)⁺, 403.04; found, 403.0 |
| Example 290 | 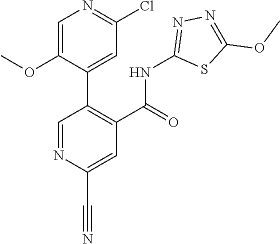 2′-chloro-6-cyano-5′-methoxy-N- (5-methoxy-1,3,4-thiadiazol-2-yl)- [3,4′-bipyridine]-4-carboxamide | 1H-NMR (400 MHz, DMSO-d6): δ 13.24 (brs, 1H), 8.93 (s, 1H), 8.48 (s, 1H), 8.22 (s, 1H), 7.70 (s, 1H), 4.09 (s, 3H), 3.63 (s, 3H). | MS (ESI) calculated for (C₁₆H₁₁ClN₆O₃S) (M + 1)⁺, 403.04; found, 403.0 |
| Example 291 | 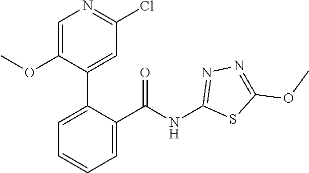 2-(2-chloro-5-methoxypyridin-4- yl)-N-(5-methoxy-1,3,4- thiadiazol-2-yl)benzamide | ¹H NMR (DMSO-d₆, 400 MHz) δ 12.73 (br s, 1H), 8.10 (s, 1H), 7.77 (dd, 1H, J = 1.2, 7.6 Hz), 7.6-7.7 (m, 1H), 7.6-7.6 (m, 1H), 7.48 (dd, 1H, J = 1.0, 7.3 Hz), 7.44 (s, 1H), 4.07 (s, 3H), 3.61 (s, 3H) | MS (ESI) calc′d for (C₁₆H₁₃ClN₄O₃S) (M + 1)⁺, 377.0, found 377.1. |
| Example 292 | 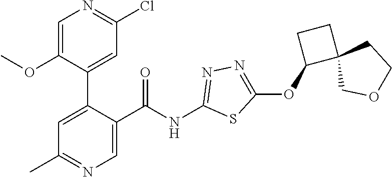 [SMILES: COc1cnc(Cl)cc1-c1cc(C)ncc1C(=O)Nc1nnc(O[C@H]2CC[C@@]23CCOC3)s1] rac-N-(5-(((1S,4S)-6- oxaspiro[3.4]octan-1-yl)oxy)- 1,3,4-thiadiazol-2-yl)-2′-chloro-5′- methoxy-6-methyl-[4,4′- bipyridine]-3-carboxamide. The stereochemistry was not determined. | 1H-NMR (400 MHz, DMSO-d⁶): δ 12.89 (s, 1H), 8.80 (s, 1H), 8.17 (s, 1H), 7.55 (s, 1H), 7.44 (s, 1H), 5.23 (t, J = 7.6 Hz, 1H), 4.02 (d, J = 8.8 Hz, 1H), 3.64-3.52 (m, 3H), 3.63 (s, 3H), 2.59 (s, 3H), 2.40- 2.32 (m, 1H), 2.31- 2.21 (m, 1H), 2.05- 1.97 (m, 1H), 1.97- 1.85 (m, 2H), 1.82- 1.72 (m, 1H). | MS (ESI) calculated for (C₂₂H₂₂ClN₅O₄S) (M + 1)⁺, 488.12; found, 488.0. |
| Example 293 | 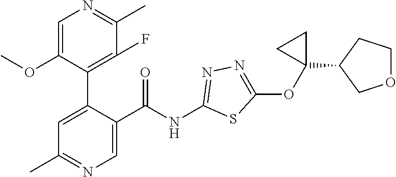 [SMILES: COc1cnc(C)c(F)c1-c1cc(C)ncc1C(=O)Nc1nnc(OC2([C@@H]3CCOC3)CC2)s1] 3′-fluoro-5′-methoxy-2′,6- dimethyl-N-(5-(1-((R)- tetrahydrofuran-3- yl)cyclopropoxy)-1,3,4-thiadiazol- 2-yl)-[4,4′-bipyridine]-3- carboxamide. The stereochemistry was not determined. | 1H-NMR (400 MHz, DMSO-d⁶): δ 13.06 (s, 1H), 8.93 (s, 1H), 8.18 (s, 1H), 7.37 (s, 1H), 3.79-3.71 (m, 2H), 3.69 (s, 3H), 3.61 (q, J = 7.60 Hz, 1H), 3.42-3.36 (m, 1H), 3.10 (t, J = 7.60 Hz, 1H), 2.58 (s, 3H), 2.43 (d, J = 2.80 Hz, 3H), 2.00-1.90 (m, 1H), 1.60-1.50 (m, 1H), 1.10-1.02 (m, 2H), 0.98-0.90 (m, 2H), | MS (ESI) calculated for (C₂₃H₂₄FN₅O₄S) (M + 1)⁺, 486.16; found, 486.2. |
| Example 295 | 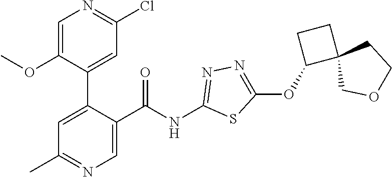 [SMILES: COc1cnc(Cl)cc1-c1cc(C)ncc1C(=O)Nc1nnc(O[C@@H]2CC[C@@]23CCOC3)s1] rac-N-(5-(((1R,4S)-6- oxaspiro[3.4]octan-1-yl)oxy)- 1,3,4-thiadiazol-2-yl)-2′-chloro-5′- methoxy-6-methyl-[4,4′- bipyridine]-3-carboxamide. The stereochemistry was not determined. | 1H-NMR (400 MHz, DMSO-d⁶): δ 8.80 (s, 1H), 8.17 (s, 1H), 7.55 (s, 1H), 7.44 (s, 1H), 5.19 (t, J = 7.6 Hz, 1H), 3.91 (d, J = 8.8 Hz, 1H), 3.72-3.58 (m, 3H), 3.63 (s, 3H), 2.59 (s, 3H), 2.40- 2.30 (m, 1H), 2.28- 2.19 (m, 1H), 2.12- 2.00 (m, 1H), 1.90- 1.80 (m, 2H), 1.78- 1.69 (m, 1H). | MS (ESI) calculated for (C₂₂H₂₂ClN₅O₄S) (M + 1)⁺, 488.12; found, 488.0. |
| Example 296 | 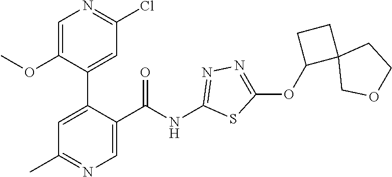 [SMILES: COc1cnc(Cl)cc1-c1cc(C)ncc1C(=O)Nc1nnc(OC2CCC23CCOC3)s1] N-(5-((6-oxaspiro[3.4]octan-1- yl)oxy)-1,3,4-thiadiazol-2-yl)-2′- chloro-5′-methoxy-6-methyl-[4,4′- bipyridine]-3-carboxamide | | MS (ESI) calculated for (C₂₂H₂₂ClN₅O₄S) (M + 1)⁺, 488.12; found, 488.0. |
| Example 297 | 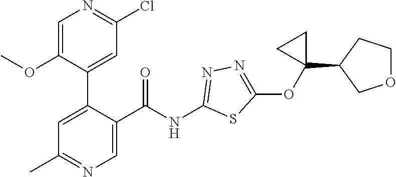 [SMILES: COc1cnc(Cl)cc1-c1cc(C)ncc1C(=O)Nc1nnc(OC2([C@H]3CCOC3)CC2)s1] (S)-2′-chloro-5′-methoxy-6- methyl-N-(5-(1-(tetrahydrofuran- 3-yl)cyclopropoxy)-1,3,4- thiadiazol-2-yl)-[4,4′-bipyridine]- 3-carboxamide. The stereochemistry was not determined. | 1H-NMR (400 MHz, DMSO-d⁶): δ 13.05 (brs, 1H), 8.81 (s, 1H), 8.19 (s, 1H), 7.55 (s, 1H), 7.44 (s, 1H), 3.79-3.70 (m, 2H), 3.69-3.58 (m, 4H), 3.42-3.35 (m, 1H), 3.15-3.05 (m, 1H), 2.59 (s, 3H), 2.02- 1.90 (m, 1H), 1.62- 1.51 (m, 1H), 1.14- 1.04 (m, 2H), 0.99- 0.89 (m, 2H), | MS (ESI) calculated for (C₂₂H₂₂ClN₅O₄S) (M + 1)⁺, 488.12; found, 488.0. |
| Example 299 | 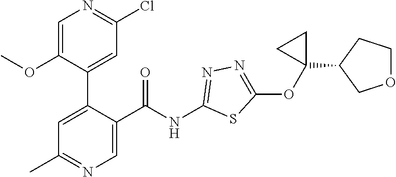 [SMILES: COc1cnc(Cl)cc1-c1cc(C)ncc1C(=O)Nc1nnc(OC2([C@@H]3CCOC3)CC2)s1] (R)-2′-chloro-5′-methoxy-6- methyl-N-(5-(1-(tetrahydrofuran- 3-yl)cyclopropoxy)-1,3,4- thiadiazol-2-yl)-[4,4′-bipyridine]- 3-carboxamide. The stereochemistry was not determined. | 1H-NMR (400 MHz, DMSO-d⁶): δ 12.98 (brs, 1H), 8.81 (s, 1H), 8.19 (s, 1H), 7.55 (s, 1H), 7.44 (s, 1H), 3.80-3.70 (m, 2H), 3.69-3.58 (m, 4H), 3.42-3.36 (m, 1H), 3.15-3.05 (m, 1H), 2.59 (s, 3H), 2.02- 1.90 (m, 1H), 1.62- 1.51 (m, 1H), 1.13- 1.05 (m, 2H), 0.99- 0.89 (m, 2H). | MS (ESI) calculated for (C₂₂H₂₂ClN₅O₄S) (M + 1)⁺, 488.12; found, 488.0. |
| Example 300 | 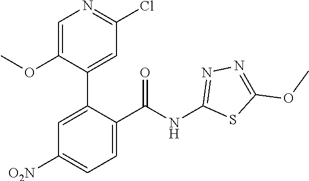 2-(2-chloro-5-methoxypyridin-4- yl)-N-(5-methoxy-1,3,4- thiadiazol-2-yl)-4-nitrobenzamide | ¹H NMR (DMSO-d₆, 400 MHz) δ 13.09 (br s, 1H), 8.42 (dd, 1H, J = 2.2, 8.6 Hz), 8.29 (d, 1H, J = 2.4 Hz), 8.17 (s, 1H), 8.04 (d, 1H, J = 8.8 Hz), 7.62 (s, 1H), 4.08 (s, 3H), 3.63 (s, 3H) | MS (ESI) calc′d for (C₁₆H₁₂ClN₅O₅S) (M + 1)⁺, 422.0, found 422.1. |
| Example 301 | 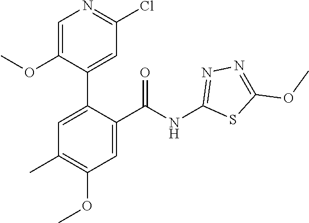 2-(2-chloro-5-methoxypyridin-4- yl)-5-methoxy-N-(5-methoxy- 1,3,4-thiadiazol-2-yl)-4- methylbenzamide | ¹H NMR (DMSO-d₆, 400 MHz) δ 12.65 (s, 1H), 8.05 (s, 1H), 7.38 (s, 1H), 7.36 (s, 1H), 7.26 (d, 1H, J = 1.0 Hz), 4.07 (s, 3H), 3.94 (s, 3H), 3.58 (s, 3H), 2.23 (s, 3H) | MS (ESI) calc′d for (C₁₈H₁₇ClN₄O₄S) (M + 1)⁺, 421.1, found 421.1. |
| Example 302 | 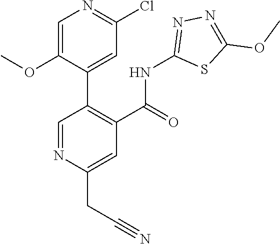 2′-chloro-6-(cyanomethyl)-5′- methoxy-N-(5-methoxy-1,3,4- thiadiazol-2-yl)-[3,4′-bipyridine]- 4-carboxamide | 1H-NMR (400 MHz, DMSO-d⁶): δ 13.08 (s, 1H), 8.72 (s, 1H), 8.17 (s, 1H), 7.80 (s, 1H), 7.63 (s, 1H), 4.40 (s, 2H), 4.09 (s, 3H), 3.62 (s, 3H). | MS (ESI) calculated for (C₁₇H₁₃ClN₆O₃S) (M + 1)⁺, 417.05; found, 417.0. |
| Example 303 | 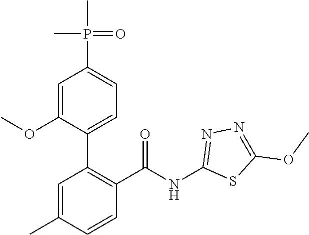 4-(4-(dimethylphosphoryl)-2- methoxyphenyl)-N-(5-methoxy- 1,3,4-thiadiazol-2-yl)-6- methylnicotinamide | 1H-NMR (400 MHz, DMSO-d⁶): δ 12.82 (brs, 1H), 8.71 (s, 1H), 7.49-7.45 (m, 2H), 7.33-7.29 (m, 2H), 4.07 (s, 3H), 3.58 (s, 3H), 2.58 (s, 3H), 1.71 (s, 3H), 1.68 (s, 3H) | MS (ESI) calculated for (C₁₉H₂₁N₄O₄PS) (M + 1)⁺, 433.11; found, 433.2 |
| Example 304 | 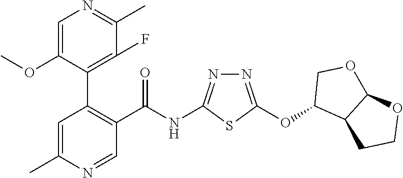 [SMILES: COc1cnc(C)c(F)c1-c1cc(C)ncc1C(=O)Nc1nnc(O[C@@H]2CO[C@H]3OCC[C@H]32)s1] 3′-fluoro-N-(5-(((3S,3aS,6aR)- hexahydrofuro[2,3-b]furan-3- yl)oxy)-1,3,4-thiadiazol-2-yl)-5′- methoxy-2′,6-dimethyl-[4,4′- bipyridine]-3-carboxamide | ¹H NMR (400 MHz, DMSO-d₆) δ 13.08- 12.89 (m, 1H), 8.89 (s, 1H), 8.17 (s, 1H), 7.40 (s, 1H), 5.77 (d, J = 4.9 Hz, 1H), 5.36 (s, 1H), 4.04 (d, J = 2.4 Hz, 2H), 3.84- 3.69 (m, 2H), 3.68 (s, 3H), 3.08-2.99 (m, 1H), 2.58 (s, 3H), 2.42 (d, J = 2.9 Hz, 3H), 2.18-2.06 (m, 1H), 1.95 (ddt, J = 13.1, 6.6, 3.4 Hz, 1H) | MS (ESI) calc′d for (C₂₂H₂₂FN₅O₅S) (M + 1)⁺, 488.1, found 488.1. |
| Example 305 | 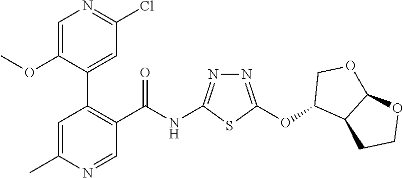 [SMILES: COc1cnc(Cl)cc1-c1cc(C)ncc1C(=O)Nc1nnc(O[C@@H]2CO[C@H]3OCC[C@H]32)s1] 2′-chloro-N-(5-(((3S,3aS,6aR)- hexahydrofuro[2,3-b]furan-3- yl)oxy)-1,3,4-thiadiazol-2-yl)-5′- methoxy-6-methyl-[4,4′- bipyridine]-3-carboxamide | ¹H NMR (400 MHz, DMSO-d₆) δ 13.00- 12.76 (m, 1H), 8.79 (s, 1H), 8.17 (s, 1H), 7.54 (s, 1H), 7.44 (s, 1H), 5.78 (d, J = 5.4 Hz, 1H), 5.37 (d, J = 2.0 Hz, 1H), 4.08- 4.02 (m, 2H), 3.85- 3.69 (m, 2H), 3.62 (s, 3H), 3.09-2.98 (m, 1H), 2.59 (s, 3H), 2.18-2.08 (m, 1H), 1.95 (ddt, J = 13.1, 6.6, 3.4 Hz, 1H) | MS (ESI) calc′d for (C₂₁H₂₀ClN₅O₅S) (M + 1)⁺, 490.1, found 490.1. |
| Example 306 | 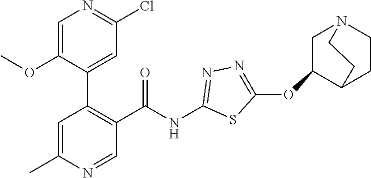 [SMILES: COc1cnc(Cl)cc1-c1cc(C)ncc1C(=O)Nc1nnc(O[C@H]2CN3CCC2CC3)s1] (R)-2′-chloro-5′-methoxy-6- methyl- N-(5-(quinuclidin-3-yloxy)-1,3,4- thiadiazol-2-yl)-[4,4′-bipyridine]- 3-carboxamide. | 1H NMR (400 MHz, METHANOL-d4) δ ppm 1.59-1.73 (m, 1 H), 1.73-1.86 (m, 1 H), 1.86-1.99 (m, 1 H), 2.00-2.13 (m, 1 H), 2.37-2.46 (m, 1 H), 2.66 (s, 3 H), 2.89- 3.14 (m, 5 H), 3.46- 3.58 (m, 1 H), 3.73 (s, 3 H), 5.05-5.17 (m, 1 H), 7.38 (s, 1 H), 7.44-7.48 (m, 1 H), 8.03-8.09 (m, 1 H), 8.79-8.85 (m, 1 H) | MS (ESI) calculated for (C₂₂H₂₃ClN₆O₃S) (M + 1)⁺, 487.1; found, 487.0, 489.0 |
| Example 307 | 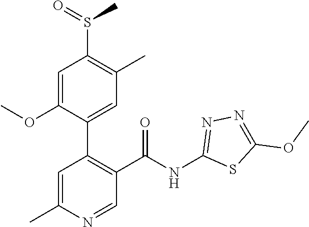 (S)-N-(5-methoxy-1,3,4- thiadiazol-2-yl)-4-(2-methoxy-5- methyl-4-(methylsulfinyl)phenyl)- 6-methylnicotinamide. The stereochemistry was not determined. | 1H-NMR (400 MHz, DMSO-d⁶): δ 12.72 (brs, 1H), 8.73 (s, 1H), 7.34-7.25 (m, 3H), 4.05 (s, 3H), 3.56 (s, 3H), 2.74 (s, 3H), 2.57 (s, 3H), 2.33 (s, 3H). | MS (ESI) calculated for (C₁₉H₂₀N₄O₄S₂) (M + 1)⁺, 433.10; found, 433.0 |
| Example 308 | 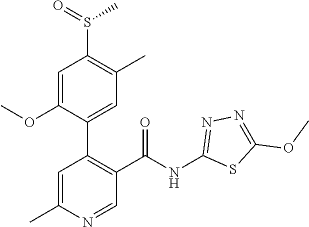 (R)-N-(5-methoxy-1,3,4- thiadiazol-2-yl)-4-(2-methoxy-5- methyl-4-(methylsulfinyl)phenyl)- 6-methylnicotinamide. The stereochemistry was not determined. | 1H-NMR (400 MHz, DMSO-d⁶): δ 12.83 (brs, 1H), 8.73 (s, 1H), 7.35-7.26 (m, 3H), 4.05 (s, 3H), 3.56 (s, 3H), 2.74 (s, 3H), 2.57 (s, 3H), 2.33 (s, 3H). | MS (ESI) calculated for (C₁₉H₂₀N₄O₄S₂) (M + 1)⁺, 433.10; found, 433.1 |
| Example 309 | 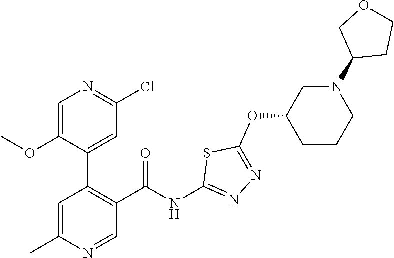 [SMILES: COc1cnc(Cl)cc1-c1cc(C)ncc1C(=O)Nc1nnc(O[C@H]2CCCN([C@@H]3CCOC3)C2)s1] 2′-chloro-5′-methoxy-6-methyl-N- (5-(((S)-1-((R)-tetrahydrofuran-3- yl)piperidin-3-yl)oxy)-1,3,4- thiadiazol-2-yl)-[4,4′-bipyridine]- 3-carboxamide. The stereochemistry was determined using VCD.. | ¹H NMR (400 MHz, DMSO-d₆) δ 12.88 (br s, 1H), 8.80 (s, 1H), 8.17 (s, 1H), 7.53 (s, 1H), 7.43 (s, 1H), 4.91 (dt, 1H), 3.81-3.68 (m, 2H), 3.63 (s, 3H), 3.62- 3.57 (m, 1H), 3.48 (dd, J = 8.6, 6.1 Hz, 1H), 3.08-2.97 (m, 1H), 2.91-2.79 (m, 1H), 2.59 (s, 4H), 2.48-2.41 (m, 1H), 2.28-2.16 (m, 1H), 2.07-1.89 (m, 2H), 1.82-1.65 (m, 2H), 1.63-1.44 (m, 2H) | MS (ESI) calc′d for (C₂₄H₂₇ClN₆O₄S) (M + 1)⁺, 531.0, found 531.1 |
| Example 310 | 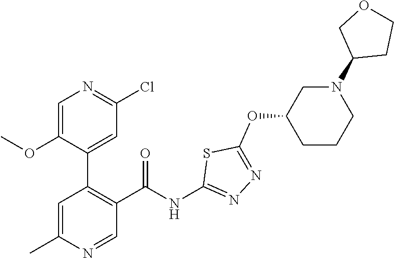 [SMILES: COc1cnc(Cl)cc1-c1cc(C)ncc1C(=O)Nc1nnc(O[C@H]2CCCN([C@@H]3CCOC3)C2)s1] 2′-chloro-5′-methoxy-6-methyl-N- (5-(((S)-1-((S)-tetrahydrofuran-3- yl)piperidin-3-yl)oxy)-1,3,4- thiadiazol-2-yl)-[4,4′-bipyridine]- 3-carboxamide. The stereochemistry was determined using VCD. | ¹H NMR (400 MHz, DMSO-d₆) δ 12.89 (br d, J = 4.4 Hz, 1H), 8.80 (s, 1H), 8.17 (s, 1H), 7.53 (s, 1H), 7.43 (s, 1H), 5.00-4.86 (m, 1H), 3.78 (td, J = 8.4, 4.2 Hz, 1H), 3.74-3.69 (m, 1H), 3.63 (s, 3H), 3.62-3.57 (m, 1H), 3.46 (dd, J = 8.6, 6.1 Hz, 1H), 3.10-2.94 (m, 2H), 2.59 (s, 3H), 2.49-2.42 (m, 1H), 2.41-2.16 (m, 2H), 2.08-1.89 (m, 2H), 1.82-1.68 (m, 2H), 1.65-1.42 (m, 2H) | MS (ESI) calc′d for (C₂₄H₂₇ClN₆O₄S) (M + 1)⁺, 531.0, found 531.1 |
| Example 314 | 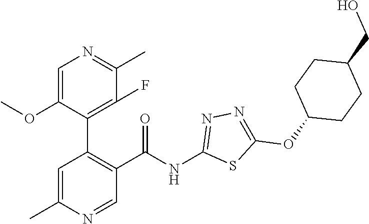 [SMILES: COc1cnc(C)c(F)c1-c1cc(C)ncc1C(=O)Nc1nnc(O[C@H]2CC[C@H](CO)CC2)s1] 3′-fluoro-N-(5-(((1r,4r)-4- (hydroxymethyl)cyclohexyl)oxy)- 1,3,4-thiadiazol-2-yl)-5′-methoxy- 2′,6-dimethyl-[4,4′-bipyridine]-3- carboxamide. The relative stereochemistry was determined as synthesis used starting material with known relative stereochemistry. | 1H-NMR (400 MHz, DMSO-d⁶): δ 12.96 (s, 1H), 8.89 (s, 1H), 8.18 (s, 1H), 7.40 (s, 1H), 4.85-4.75 (m, 1H), 4.46 (t, J = 5.2 Hz, 1H), 3.68 (s, 3H), 3.23 (t, J = 5.6 Hz, 2H), 2.59 (s, 3H), 2.43 (d, J = 2.80 Hz, 3H), 2.23-2.15 (m, 2H), 1.85-1.77 (m, 2H), 1.50-1.32 (m, 3H), 1.10-0.98 (m, 2H). | MS (ESI) calculated for (C₂₃H₂₆FN₅O₄S) (M + 1)⁺, 488.18; found, 488.2. |
| Example 315, | 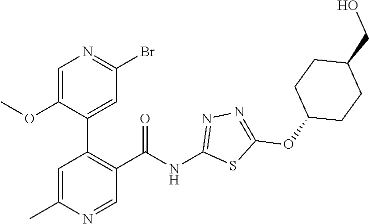 [SMILES: COc1cnc(Br)cc1-c1cc(C)ncc1C(=O)Nc1nnc(O[C@H]2CC[C@H](CO)CC2)s1] 2′-bromo-N-(5-(((1r,4r)-4- (hydroxymethyl)cyclohexyl)oxy)- 1,3,4-thiadiazol-2-yl)-5′-methoxy- 6-methyl-[4,4′-bipyridine]-3- carboxamide. The relative stereochemistry was determined as synthesis used starting material with known relative stereochemistry. | 1H-NMR (400 MHz, DMSO-d⁶): δ 12.88 (s, 1H), 8.79 (s, 1H), 8.18 (s, 1H), 7.64 (s, 1H), 7.43 (s, 1H), 4.85-4.75 (m, 1H), 4.46 (t, J = 5.20 Hz, 1H), 3.62 (s, 3H), 3.18 (t, J = 5.20 Hz, 2H), 2.69 (s, 3H), 2.25- 2.15 (m, 2H), 1.87- 1.76 (m, 2H), 1.50- 1.35 (m, 3H), 1.11- 0.98 (m, 2H). | MS (ESI) calculated for (C₂₂H₂₄BrN₅O₄S) (M + 1)⁺, 534.08; found, 534.0. |
| Example 316 | 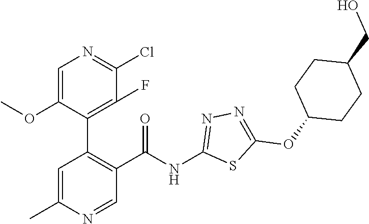 [SMILES: COc1cnc(Cl)c(F)c1-c1cc(C)ncc1C(=O)Nc1nnc(O[C@H]2CC[C@H](CO)CC2)s1] 2′-chloro-3′-fluoro-N-(5-(((1r,4r)-4- (hydroxymethyl)cyclohexyl)oxy)- 1,3,4-thiadiazol-2-yl)-5′-methoxy- 6-methyl-[4,4′-bipyridine]-3- carboxamide. The relative stereochemistry was determined as synthesis used starting material with known relative stereochemistry. | 1H-NMR (400 MHz, DMSO-d⁶): δ 13.03 (s, 1H), 8.98 (s, 1H), 8.18 (s, 1H), 7.46 (s, 1H), 4.82-4.73 (m, 1H), 4.46 (t, J = 5.2 Hz, 1H), 3.74 (s, 3H), 3.23 (t, J = 5.6 Hz, 2H), 2.59 (s, 3H), 2.25-2.15 (m, 2H), 1.85-1.77 (m, 2H), 1.50-1.31 (m, 3H), 1.11-0.99 (m, 2H). | MS (ESI) calculated for (C₂₂H₂₃ClFN₅O₄S) (M + 1)⁺, 508.12; found, 508.1. |
| Example 317 | 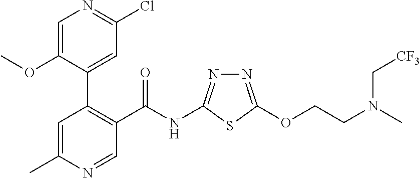 [SMILES: COc1cnc(Cl)cc1-c1cc(C)ncc1C(=O)Nc1nnc(OCCN(C)CC(F)(F)F)s1] 2′-chloro-5′-methoxy-6-methyl-N- (5-(2-(methyl(2,2,2- trifluoroethyl)amino)ethoxy)- 1,3,4-thiadiazol-2-yl)-[4,4′- bipyridine]-3-carboxamide | ¹H NMR (400 MHz, DMSO-d₆) δ 12.80 (s, 1H), 8.71 (s, 1H), 8.08 (s, 1H), 7.46 (s, 1H), 7.35 (s, 1H), 4.41 (t, J = 5.6 Hz, 2H), 3.54 (s, 3H), 3.22-3.16 (m, 2H), 2.90 (t, J = 5.6 Hz, 2H), 2.41 (t, J = 2.0 Hz, 3H), 2.36 (s, 3H). | MS (ESI) calc′d for (C₂₀H₂₀ClF₃N₆O₃S) (M + 1)⁺, 517.1, found 517.2. |
| Example 324 | 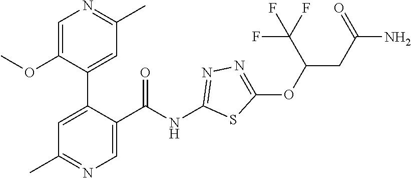 [SMILES: COc1cnc(C)cc1-c1cc(C)ncc1C(=O)Nc1nnc(OC(CC(N)=O)C(F)(F)F)s1] N-(5-((4-amino-1,1,1-trifluoro-4- oxobutan-2-yl)oxy)-1,3,4- thiadiazol-2-yl)-5′-methoxy-2′,6- dimethyl-[4,4′-bipyridine]-3- carboxamide. | ¹H NMR (700 MHz, DMSO-d₆) δ ppm 13.48 (br s, 1 H), 12.31-12.86 (m, 2 H), 8.79 (s, 1 H), 8.18 (s, 1 H), 7.40 (s, 1 H), 7.28 (s, 1 H), 6.42- 6.46 (m, 1 H), 3.54 (s, 3 H), 2.60 (s, 5 H), 2.48 (s, 3 H). | MS (ESI) calc′d for (C₂₀H₁₉F₃N₆O₄S) (M + 1)⁺, 497.47, found 497.2 |

Following procedures similar to the palladium-catalyzed coupling procedure described in Example 377. Step 3, the following examples were synthesized.

| Example | Structure/Name | LCMS |
|---|---|---|
| Example 326 | 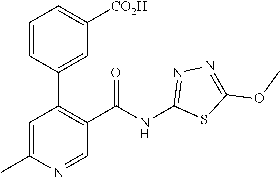 | MS (ESI) calculated for (C₁₇H₁₄N₄O₄S) (M + 1)⁺, 371.1; found, 371.1. |
| | 3-(5-((5-methoxy-1,3,4-thiadiazol-2-yl)carbamoyl)-2- | |
| | methylpyridin-4-yl)benzoic acid | |
| Example 327 | 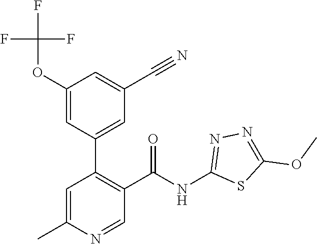 [SMILES: COc1nnc(NC(=O)c2cnc(C)cc2-c2cc(C#N)cc(OC(F)(F)F)c2)s1] | MS (ESI) calculated for (C₁₈H₁₂F₃N₅O₃S) (M + 1)⁺, 436.1; found, 436.1. |
| | 4-[3-cyano-5-(trifluoromethoxy)phenyl]-N-(5-methoxy- | |
| | 1,3,4-thiadiazol-2-yl)-6-methylpyridine-3-carboxamide | |
| Example 328 | 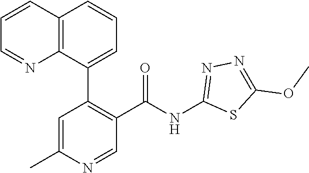 [SMILES: COc1nnc(NC(=O)c2cnc(C)cc2-c2cccc3cccnc23)s1] | MS (ESI) calc'd for (C₁₉H₁₅N₅O₂S) (M + 1)⁺, 378.1, found 378.0. |
| | N-(5-methoxy-1,3,4-thiadiazol-2-yl)-6-methyl-4- | |
| | (quinolin-8-yl)nicotinamide | |
| Example 329 | 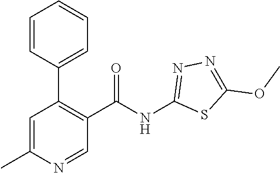 | MS (ESI) calc'd for (C₁₆H₁₄N₄O₂S) (M + 1)⁺, 327.1, found 327.1 |
| | N-(5-methoxy-1,3,4-thiadiazol-2-yl)-6-methyl-4- | |
| | phenylpyridine-3-carboxamide | |
| Example 330 | 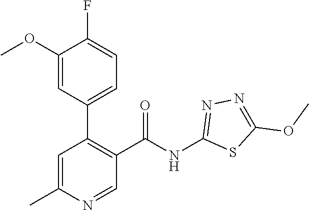 | MS (ESI) calc'd for (C₁₇H₁₅FN₄O₃S) (M + Na)⁺, 397.1 found 397.2 [M + Na]+ |
| | 4-(4-fluoro-3-methoxyphenyl)-N-(5-methoxy-1,3,4- | |
| | thiadiazol-2-yl)-6-methylpyridine-3-carboxamide | |
| Example 331 | 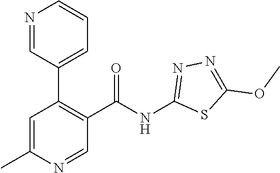 | MS (ESI) calc'd for (C₁₅H₁₃N₅O₂S) (M + 1)⁺, 328.1, found 328.2. |
| | N-(5-methoxy-1,3,4-thiadiazol-2-yl)-6′-methyl-[3,4′- | |
| | bipyridine]-3′-carboxamide | |
| Example 332 | 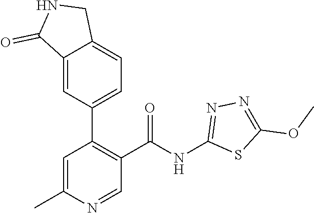 | MS (ESI) calc'd for (C₁₈H₁₅N₅O₃S) (M + 1)⁺, 328.1, found 328.1. |
| | N-(5-methoxy-1,3,4-thiadiazol-2-yl)-6-methyl-4-(3-oxo- | |
| | 2,3-dihydro-1H-isoindol-5-yl)pyridine-3-carboxamide | |
| Example 333 | 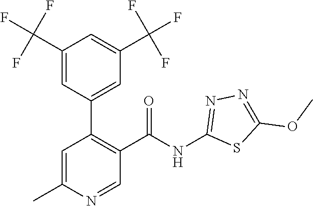 [SMILES: COc1nnc(NC(=O)c2cnc(C)cc2-c2cc(C(F)(F)F)cc(C(F)(F)F)c2)s1] | MS (ESI) calc'd for (C₁₈H₁₂F₆N₄O₂S) (M + 1)⁺, 463.1, found 463.2. |
| | 4-[3,5-bis(trifluoromethyl)phenyl]-N-(5-methoxy-1,3,4- | |
| | thiadiazol-2-yl)-6-methylpyridine-3-carboxamide | |
| Example 334 | 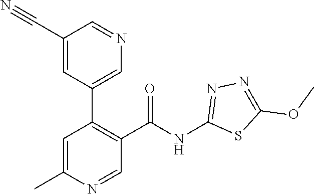 [SMILES: COc1nnc(NC(=O)c2cnc(C)cc2-c2cncc(C#N)c2)s1] | MS (ESI) calc'd for (C₁₆H₁₂N₆O₂S) (M + 1)⁺, 353.1, found 353.1. |
| | 5-cyano-N-(5-methoxy-1,3,4-thiadiazol-2-yl)-6′-methyl- | |
| | [3,4′-bipyridine]-3′-carboxamide | |
| Example 335 | 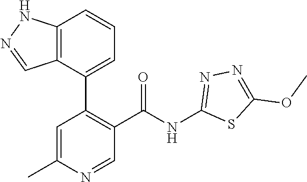 [SMILES: COc1nnc(NC(=O)c2cnc(C)cc2-c2cccc3[nH]ncc23)s1] | MS (ESI) calc'd for (C₁₇H₁₄N₆O₂S) (M + 1)⁺, 367.1, found 367.1. |
| | 4-(1H-indazol-4-yl)-N-(5-methoxy-1,3,4-thiadiazol-2-yl)- | |
| | 6-methylnicotinamide | |
| Example 336 | 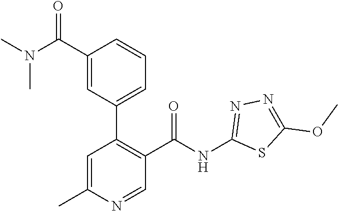 | MS (ESI) calc'd for (C₁₉H₁₉N₅O₃S) (M + 1)⁺, 398.1, found 398.2. |
| | 4-[3-(dimethylcarbamoyl)phenyl]-N-(5-methoxy-1,3,4- | |
| | thiadiazol-2-yl)-6-methylpyridine-3-carboxamide | |
| Example 337 | 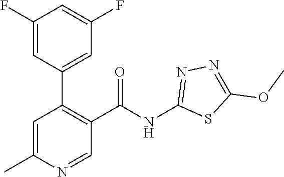 | MS (ESI) calc'd for (C₁₆H₁₂F₂N4O2S) (M + 1)⁺, 363.1, found 363.1. |
| | 4-(3,5-difluorophenyl)-N-(5-methoxy-1,3,4-thiadiazol-2- | |
| | yl)-6-methylpyridine-3-carboxamide | |
| Example 338 | 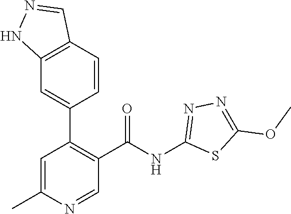 | MS (ESI) calc'd for (C₁₇H₁₄N₆O₂S) (M + 1)⁺, 367.1, found 367.1. |
| | 4-(1H-indazol-6-yl)-N-(5-methoxy-1,3,4-thiadiazol-2-yl)- | |
| | 6-methylnicotinamide | |
| Example 339 | 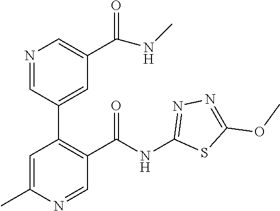 | MS (ESI) calc'd for (C₁₇H₁₆N₆O₃S) (M + 1)⁺, 385.1, found 385.1. |
| | N3′-(5-methoxy-1,3,4-thiadiazol-2-yl)-N5,6′-dimethyl- | |
| | [3,4′-bipyridine]-3′,5-dicarboxamide | |
| Example 340 | 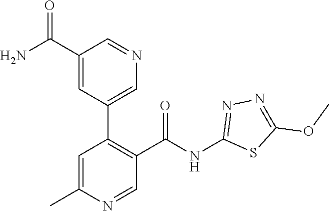 [SMILES: COc1nnc(NC(=O)c2cnc(C)cc2-c2cncc(C(N)=O)c2)s1] | MS (ESI) calc'd for (C₁₆H₁₄N₆O₃S) (M + 1)⁺, 371.1, found 371.1. |
| | N3′-(5-methoxy-1,3,4-thiadiazol-2-yl)-6′-methyl-[3,4′- | |
| | bipyridine]-3′,5-dicarboxamide | |
| Example 341 | 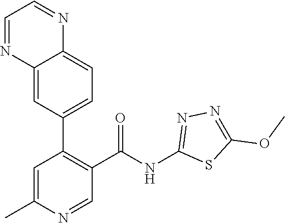 | MS (ESI) calc'd for (C₁₈H₁₄N₆O₂S) (M + 1)⁺, 379.1, found 379.1. |
| | N-(5-methoxy-1,3,4-thiadiazol-2-yl)-6-methyl-4- | |
| | (quinoxalin-6-yl)pyridine-3-carboxamide | |
| Example 342 | 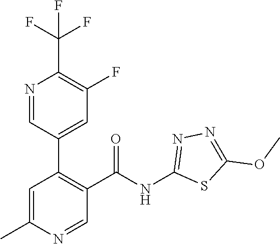 | MS (ESI) calc'd for (C₁₆H₁₁F₄N₅O₂S) (M + 1)⁺, 414.1, found 414.1. |
| | 5-fluoro-N-(5-methoxy-1,3,4-thiadiazol-2-yl)-6′-methyl- | |
| | 6-(trifluoromethyl)-[3,4′-bipyridine]-3′-carboxamide | |
| Example 343 | 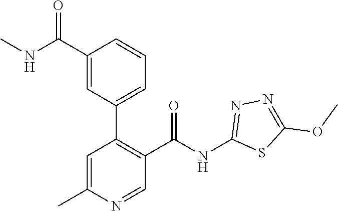 | MS (ESI) calc'd for (C₁₈H₁₇N₅O₃S) (M + 1)⁺, 384.1, found 384.1. |
| | N-(5-methoxy-1,3,4-thiadiazol-2-yl)-6-methyl-4-[3- | |
| | (methylcarbamoyl)phenyl]pyridine-3-carboxamide | |
| Example 344 | 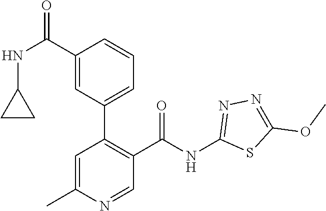 [SMILES: COc1nnc(NC(=O)c2cnc(C)cc2-c2cccc(C(=O)NC3CC3)c2)s1] | MS (ESI) calc'd for (C₂₀H₁₉N₅O₃S) (M + 1)⁺, 410.1, found 410.1. |
| | 4-[3(cyclopropylcarbamoyl)phenyl]- | |
| | N-(5-methoxy-1,3,4-thiadiazol-2-yl)-6-methylpyridine- | |
| | 3carboxamide | |
| Example 345 | 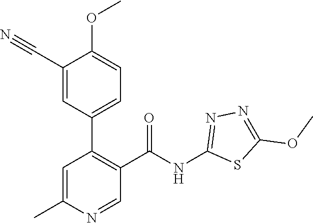 [SMILES: COc1nnc(NC(=O)c2cnc(C)cc2-c2ccc(OC)c(C#N)c2)s1] | MS (ESI) calc'd for (C₁₈H₁₅N₅O₃S) (M + 1)⁺, 382.1, found 382.0. |
| | 4-(3-cyano-4-methoxyphenyl)-N-(5-methoxy-1,3,4- | |
| | thiadiazol-2-yl)-6-methylpyridine-3-carboxamide | |
| Example 346 | 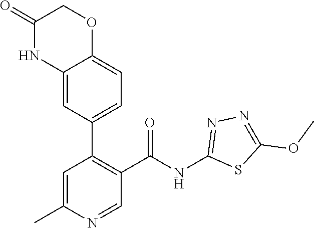 [SMILES: COc1nnc(NC(=O)c2cnc(C)cc2-c2ccc3c(c2)NC(=O)CO3)s1] | MS (ESI) calc'd for (C₁₈H₁₅N₅O₄S) (M + 1)⁺, 398.1, found 398.1. |
| | N-(5-methoxy-1,3,4-thiadiazol-2-yl)-6-methyl-4-(3-oxo- | |
| | 3,4-dihydro-2H-1,4-benzoxazin-6-yl)pyridine-3- | |
| | carboxamide | |
| Example 347 | 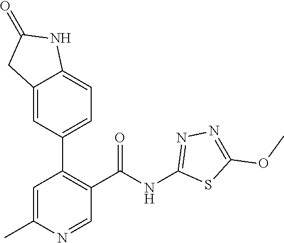 | MS (ESI) calc'd for (C₁₈H₁₅N₅O₃S) (M + 1)⁺, 382.1, found 382.1. |
| | N-(5-methoxy-1,3,4-thiadiazol-2-yl)-6-methyl-4-(2-oxo- | |
| | 2,3-dihydro-1H-indol-5-yl)pyridine-3-carboxamide | |
| Example 348 | 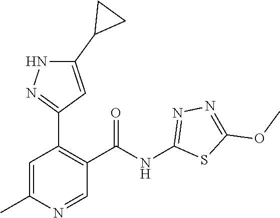 | MS (ESI) calc'd for (C₁₆H₁₆N₆O₂S) (M + 1)⁺, 357.1, found 357.1. |
| | 4-(3-cyclopropyl-1H-pyrazol-5-yl)-N-(5-methoxy-1,3,4- | |
| | thiadiazol-2-yl)-6-methylpyridine-3-carboxamide | |
| Example 349 | 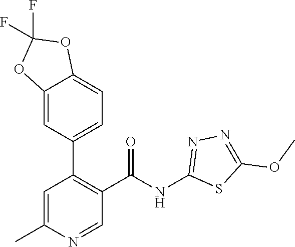 [SMILES: COc1nnc(NC(=O)c2cnc(C)cc2-c2ccc3c(c2)OC(F)(F)O3)s1] | MS (ESI) calc'd for (C₁₇H₁₂F₂N₄O₄S) (M + 1)⁺, 407.1, found 407.0. |
| | 4-(2,2-difluoro-2H-1,3-benzodioxol-5-yl)-N-(5-methoxy- | |
| | 1,3,4-thiadiazol-2-yl)-6-methylpyridine-3-carboxamide | |
| Example 350 | 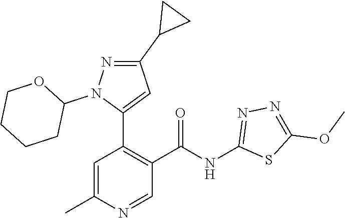 [SMILES: COc1nnc(NC(=O)c2cnc(C)cc2-c2cc(C3CC3)nn2C2CCCCO2)s1] | MS (ESI) calc'd for (C₂₁H₂₄N₆O₃S) (M + 1)⁺, 441.1, found 441.1. |
| | 4-(3-cyclopropyl-1-(tetrahydro-2H-pyran-2-yl)-1H- | |
| | pyrazol-5-yl)-N-(5-methoxy-1,3,4-thiadiazol-2-yl)-6- | |
| | methylnicotinamide | |
| Example 351 | 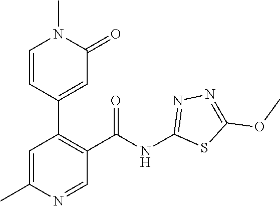 | MS (ESI) calc'd for (C₁₆H₁₅N₅O₃S) (M + 1)⁺, 358.1, found 358.1. |
| | N-(5-methoxy-1,3,4-thiadiazol-2-yl)-1′,6-dimethyl-2′- | |
| | oxo-1′,2′-dihydro-[4,4′-bipyridine]-3-carboxamide | |
| Example 352 | 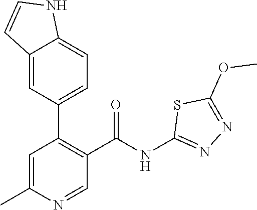 | MS (ESI) calc'd for (C₁₈H₁₅N₅O₂S) (M + 1)⁺, 366.1, found 366.1. |
| | 4-(1H-indol-5-yl)-N-(5-methoxy-1,3,4-thiadiazol-2-yl)-6- | |
| | methylpyridine-3-carboxamide | |
| Example 353 | 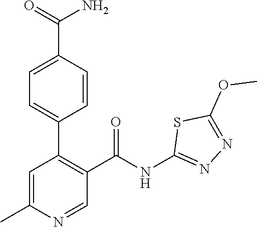 | MS (ESI) calc'd for (C₁₇H₁₅N₅O₃S) (M + 1)⁺, 370.1, found 370.1. |
| | 4-(4-carbamoylphenyl)-N-(5-methoxy-1,3,4-thiadiazol-2- | |
| | yl)-6-methylpyridine-3-carboxamide | |
| Example 354 | 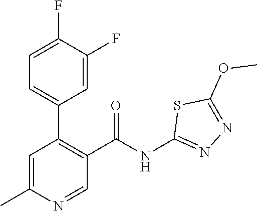 | MS (ESI) calc'd for (C₁₆H₁₂F₂N₄O₂S) (M + 1)⁺, 363.1, found 363.1. |
| | 4-(3,4-difluorophenyl)-N-(5-methoxy-1,3,4-thiadiazol-2- | |
| | yl)-6-methylpyridine-3-carboxamide | |
| Example 355 | 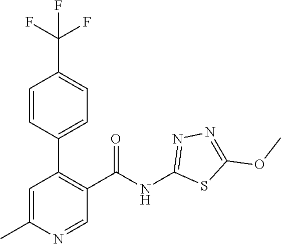 | MS (ESI) calc'd for (C₁₇H₁₃F₃N₄O₂S) (M + Na)⁺, 417.1, found 417.0. |
| | N-(5-methoxy-1,3,4-thiadiazol-2-yl)-methyl4-(4- | |
| | (trifluoromethyl) phenyl) nicotinamide | |
| Example 356 | 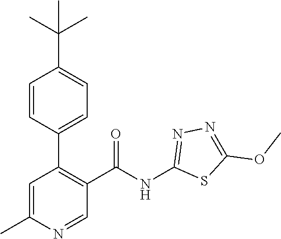 | MS (ESI) calc'd for (C₂₀H₂₂N₄O₂S) (M + 1)⁺, 383.2, found 383.2. |
| | 4-(4-(tert-butyl)phenyl)-N-(5-methoxy-1,3,4-thiadiazol-2- | |
| | yl)-6-methylnicotinamide | |
| Example 357 | 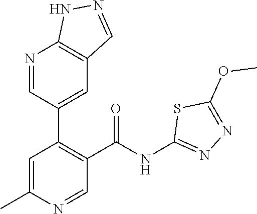 | MS (ESI) calc'd for (C₁₆H₁₃N₇O₂S) (M + 1)⁺, 368.1, found 368.1. |
| | N-(5-methoxy-1,3,4-thiadiazol-2-yl)-6-methyl-4-(1H- | |
| | pyrazolo[3,4-b]pyridin-5-yl)nicotinamide | |
| Example 358 | 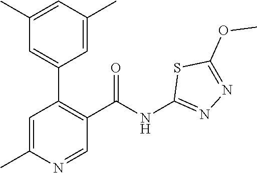 | MS (ESI) calc'd for (C₁₈H₁₈N₄O₂S) (M + 1)⁺, 355.1, found 355.2. |
| | 4-(3,5-dimethylphenyl)-N-(5-methoxy-1,3,4-thiadiazol-2- | |
| | yl)-6-methylpyridine-3-carboxamide | |
| Example 359 | 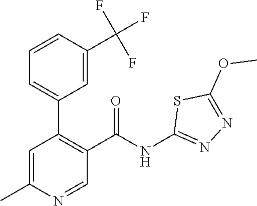 | MS (ESI) calc'd for (C₁₇H₁₃F₃N₄O₂S) (M + 1)⁺, 395.1, found 395.2. |
| | N-(5-methoxy-1,3,4-thiadiazol-2-yl)-6-methyl-4-[3- | |
| | (trifluoromethyl)phenyl]pyridine-3-carboxamide | |
| Example 360 | 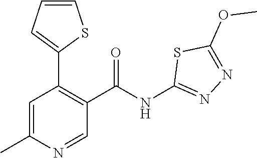 | MS (ESI) calc'd for (C₁₄H₁₃N₄O₂S₂) (M + 1), 333.0, found 333.1. |
| | N-(5-methoxy-1,3,4-thiadiazol-2-yl)-6-methyl-4- | |
| | (thiophen-2-yl)pyridine-3-carboxamide | |
| Example 361 | 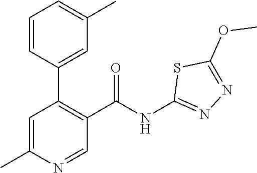 | MS (ESI) calc'd for (C₁₇H₁₆N₄O₂S) (M + 1)⁺, 341.1, found 341.1. |
| | N-(5-methoxy-1,3,4-thiadiazol-2-yl)-6-methyl-4-(3- | |
| | methylphenyl) pyridine-3-carboxamide | |
| Example 362 | 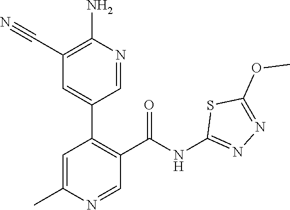 [SMILES: COc1nnc(NC(=O)c2cnc(C)cc2-c2cnc(N)c(C#N)c2)s1] | MS (ESI) calc'd for (C₁₆H₁₃N₇O₂S) (M + 1)⁺, 368.1, found 368.1. |
| | 6-amino-5-cyano-N-(5-methoxy-1,3,4-thiadiazol-2-yl)-6′- | |
| | methyl-[3,4′-bipyridine]-3′-carboxamide | |
| Example 363 | 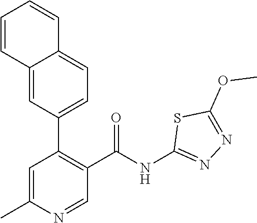 | MS (ESI) calc'd for (C₂₀H₁₆N₄O₂S) (M + 1)⁺, 377.1, found 377.1. |
| | N-(5-methoxy-1,3,4-thiadiazol-2-yl)-6-methyl-4- | |
| | (naphthalen-2-yl)pyridine-3-carboxamide | |
| Example 364 | 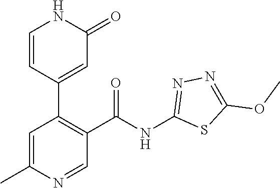 | MS (ESI) calc'd for (C₁₅H₁₃N₅O₃S) (M + 1)⁺, 344.1, found 344.2. |
| | N-(5-methoxy-1,3,4-thiadiazol-2-yl)-6-methyl-2′-oxo- | |
| | 1′,2′-dihydro-[4,4′-bipyridine]-3-carboxamide | |
| Example 365 | 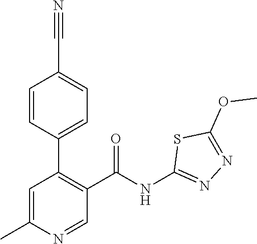 | MS (ESI) calc'd for (C₁₇H₁₃N₅O₂S) (M + 1)⁺, 352.1, found 352.1. |
| | 4-(4-cyanophenyl)-N-(5-methoxy-1,3,4-thiadiazol-2-yl)- | |
| | 6-methylpyridine-3-carboxamide | |
| Example 366 | 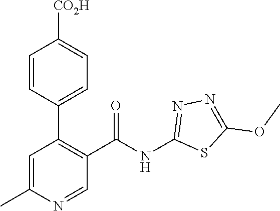 | MS (ESI) calc'd for (C₁₇H₁₄N₄O₄S) (M + 1)⁺, 371.1, found 371.1. |
| | 4-(5-((5-methoxy-1,3,4-thiadiazol-2-yl)carbamoyl)-2- | |
| | methylpyridin-4-yl)benzoic acid | |
| Example 367 | 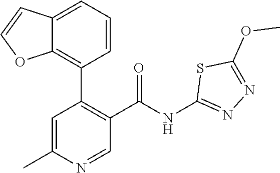 [SMILES: COc1nnc(NC(=O)c2cnc(C)cc2-c2cccc3ccoc23)s1] | MS (ESI) calc'd for (C₁₈H₁₄N₄O₃S) (M + 1)⁺, 367.1, found 367.1. |
| | 4-(1-benzofuran-7-yl)-N-(5-methoxy-1,3,4-thiadiazol-2- | |
| | yl)-6-methylpyridine-3-carboxamide | |
| Example 368 | 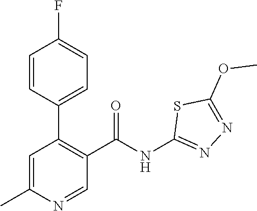 | MS (ESI) calc'd for (C₁₆H₁₃FN₄O₂S) (M + 1)⁺, 345.1, found 345.1. |
| | 4-(4-fluorophenyl)-N-(5-methoxy-1,3,4-thiadiazol-2-yl)- | |
| | 6-methylpyridine-3-carboxamide | |
| Example 369 | 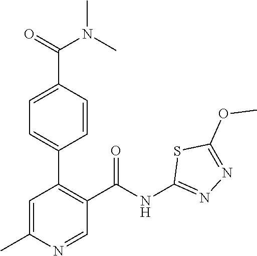 | MS (ESI) calc'd for (C₁₉H₁₉N₅O₃S) (M + 1)⁺, 398.1, found 398.2. |
| | 4-[4-(dimethylcarbamoyl)phenyl]-N-(5-methoxy-1,3,4- | |
| | thiadiazol-2-yl)-6-methylpyridine-3-carboxamide | |
| Example 370 | 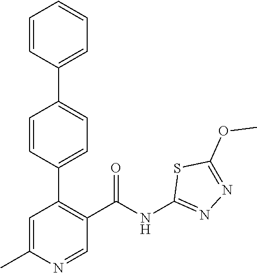 | MS (ESI) calc'd for (C₂₂H₁₈N₄O₂S) (M + 1)⁺, 403.1, found 403.2. |
| | 4-{[1,1′-biphenyl]-4-yl}-N-(5-methoxy-1,3,4-thiadiazol- | |
| | 2-yl)-6-methylpyridine-3-carboxamide | |
| Example 371 | 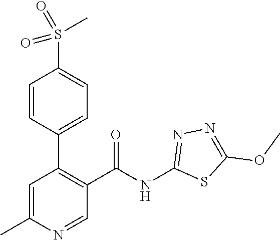 | MS (ESI) calc'd for (C₁₇H₁₆N₄O₄S₂) (M + 1)⁺, 405.1, found 405.1. |
| | N-(5-methoxy-1,3,4-thiadiazol-2-yl)-6-methyl-4-(4- | |
| | (methylsulfonyl) phenyl)nicotinamide | |
| Example 372 | 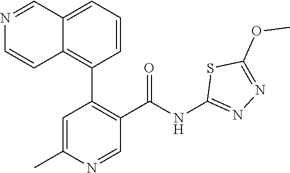 [SMILES: COc1nnc(NC(=O)c2cnc(C)cc2-c2cccc3cnccc23)s1] | MS (ESI) calc'd for (C₁₉H₁₅N₅O₂S) (M + 1)⁺, 378.1, found 378.1. |
| | 4-(isoquinolin-5-yl)-N-(5-methoxy-1,3,4-thiadiazol-2-yl)- | |
| | 6-methylpyridine-3-carboxamide | |
| Example 373 | 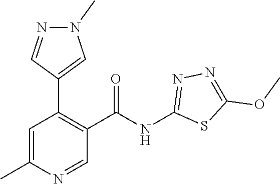 | MS (ESI) calc'd for (C₁₄H₁₄N₆O₂S) (M + 1)⁺, 331.1, found 331.1. |
| | N-(5-methoxy-1,3,4-thiadiazol-2-yl)-6-methyl-4-(1- | |
| | methyl-1H-pyrazol-4-yl)nicotinamide | |
| Example 374 | 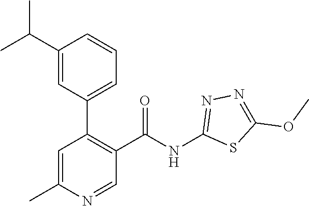 [SMILES: COc1nnc(NC(=O)c2cnc(C)cc2-c2cccc(C(C)C)c2)s1] | MS (ESI) calc'd for (C₁₉H₂₀N₄O₂S) (M + 1)⁺, 369.1, found 369.2. |
| | 4-(3-isopropylphenyl)-N-(5-methoxy-1,3,4-thiadiazol-2- | |
| | yl)-6-methylnicotinamide | |
| Example 375 | 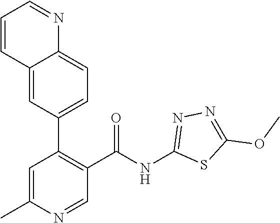 | MS (ESI) calc'd for (C₁₉H₁₅N₅O₂S) (M + 1)⁺, 378.1, found 378.1. |
| | N-(5-methoxy-1,3,4-thiadiazol-2-yl)-6-methyl-4- | |
| | (quinolin-6-yl)nicotinamide | |
| Example 376 | 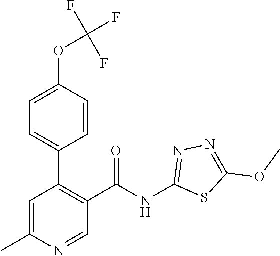 | MS (ESI) calc'd for (C₁₇H₁₃F₃N₄O₃S) (M + 1)⁺, 411.1, found 411.1. |
| | N-(5-methoxy-1,3,4-thiadiazol-2-yl)-6-methyl-4-[4- | |
| | (trifluoromethoxy)phenyl]pyridine-3-carboxamide | |
| Example 378 | 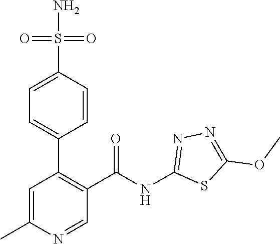 | MS (ESI) calc'd for (C₁₆H₁₅N₅O₄S₂) (M + 1)⁺, 406.1, found 406.1. |
| | N-(5-methoxy-1,3,4-thiadiazol-2-yl)-6-methyl-4-(4- | |
| | sulfamoylphenyl)pyridine-3-carboxamide | |
| Example 379 | 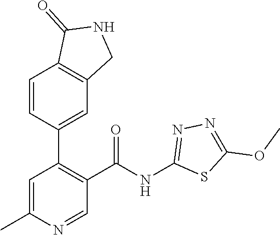 | MS (ESI) calc'd for (C₁₈H₁₅N₅O₃S) (M + 1)⁺, 382.1, found 382.1. |
| | N-(5-methoxy-1,3,4-thiadiazol-2-yl)-6-methyl-4-(1-oxo- | |
| | 2,3-dihydro-1H-isoindol-5-yl)pyridine-3-carboxamide | |
| Example 380 | 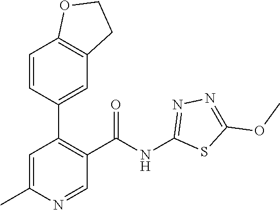 | MS (ESI) calc'd for (C₁₈H₁₆N₄O₃S) (M + 1)⁺, 369.1, found 369.1. |
| | 4-(2,3-dihydro-1-benzofuran-5-yl)-N-(5-methoxy-1,3,4- | |
| | thiadiazol-2-yl)-6-methylpyridine-3-carboxamide | |
| Example 381 | 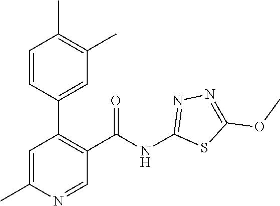 | MS (ESI) calc'd for (C₁₈H₁₈N₄O₂S) (M + 1)⁺, 355.1, found 355.2. |
| | 4-(3,4-dimethylphenyl)-N-(5-methoxy-1,3,4-thiadiazol-2- | |
| | yl)-6-methylpyridine-3-carboxamide | |
| Example 382 | 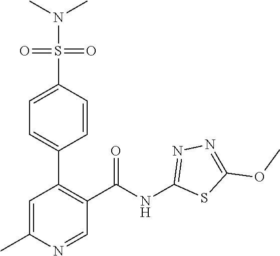 | MS (ESI) calc'd for (C₁₈H₁₉N₅O₄S₂) (M + 1)⁺, 434.1, found 434.0. |
| | 4-[4-(dimethylsulfamoyl)phenyl]-N-(5-methoxy-1,3,4- | |
| | thiadiazol-2-yl)-6-methylpyridine-3-carboxamide | |
| Example 383 | 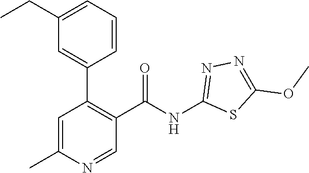 [SMILES: CCc1cccc(-c2cc(C)ncc2C(=O)Nc2nnc(OC)s2)c1] | MS (ESI) calc'd for (C₁₈H₁₈N₄O₂S) (M + 1)⁺, 355.1, found 355.0. |
| | 4-(3-ethylphenyl)-N-(5-methoxy-1,3,4-thiadiazol-2-yl)-6- | |
| | methylpyridine-3-carboxamide | |
| Example 384 | 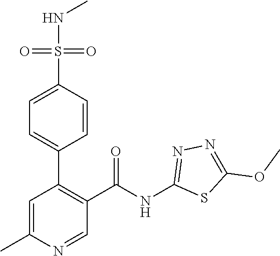 | MS (ESI) calc'd for (C₁₇H₁₇N₅O₄S₂) (M + 1)⁺, 420.1, found 420.1. |
| | N-(5-methoxy-1,3,4-thiadiazol-2-yl)-6-methyl-4-[4- | |
| | (methylsulfamoyl)phenyl]pyridine-3-carboxamide | |
| Example 385 | 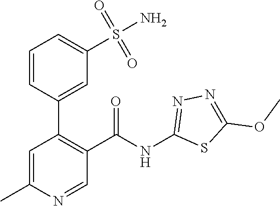 | MS (ESI) calc'd for (C₁₆H₁₅N₅O₄S₂) (M + 1)⁺, 406.1, found 406.0. |
| | N-(5-methoxy-1,3,4-thiadiazol-2-yl)-6-methyl-4-(3- | |
| | sulfamoylphenyl)pyridine-3-carboxamide | |
| Example 386 | 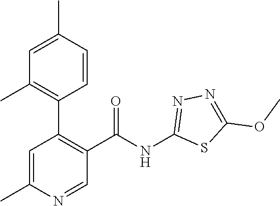 | MS (ESI) calc'd for (C₁₈H₁₈N₄O₂S) (M + 1)⁺, 355.1, found 355.1. |
| | 4-(2,4-dimethylphenyl)-N-(5-methoxy-1,3,4-thiadiazol-2- | |
| | yl)-6-methylpyridine-3-carboxamide | |
| Example 387 | 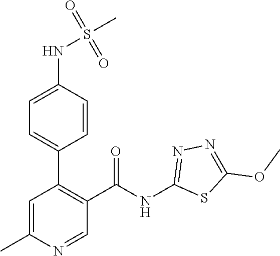 | MS (ESI) calc'd for (C₁₇H₁₇N₅O₄S₂) (M + 1)⁺, 420.1, found 420.1. |
| | 4-(4-methanesulfonamidophenyl)-N-(5-methoxy-1,3,4- | |
| | thiadiazol-2-yl)-6-methylpyridine-3-carboxamide | |
| Example 388 | 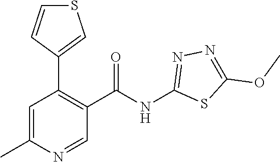 | MS (ESI) calc'd for (C₁₄H₁₂N₄O₂S₂) (M + 1)⁺, 333.0, found 333.1. |
| | N-(5-methoxy-1,3,4-thiadiazol-2-yl)-6-methyl-4- | |
| | (thiophen-3-yl)nicotinamide | |
| Example 389 | 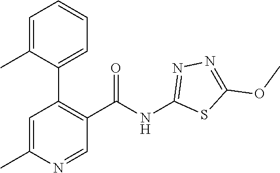 | MS (ESI) calc'd for (C₁₇H₁₆N₄O₂S) (M + 1)⁺, 341.1, found 341.1. |
| | N-(5-methoxy-1,3,4-thiadiazol-2-yl)-6-methyl-4-(2- | |
| | methylphenyl)pyridine-3-carboxamide | |
| Example 390 | 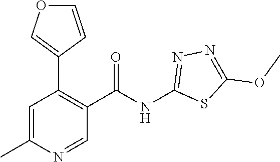 | MS (ESI) calc'd for (C₁₄H₁₂N₄O₃S) (M + 1)⁺, 317.1, found 317.1. |
| | 4-(furan-3-yl)-N-(5-methoxy-1,3,4-thiadiazol-2-yl)-6- | |
| | methylpyridine-3-carboxamide | |
| Example 391 | 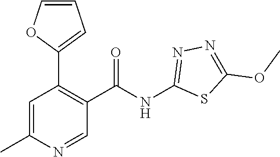 | MS (ESI) calc'd for (C₁₄H₁₂N₄O₃S) (M + 1)⁺, 317.1, found 317.0. |
| | 4-(furan-2-yl)-N-(5-methoxy-1,3,4-thiadiazol-2-yl)-6- | |
| | methylpyridine-3-carboxamide | |
| Example 392 | 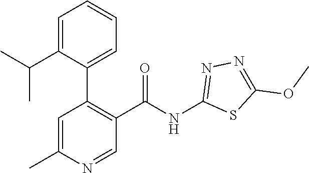 [SMILES: COc1nnc(NC(=O)c2cnc(C)cc2-c2ccccc2C(C)C)s1] | MS (ESI) calc'd for (C₁₉H₂₀N₄O₂S) (M + 1)⁺, 369.1, found 369.2. |
| | N-(5-methoxy-1,3,4-thiadiazol-2-yl)-6-methyl-4-[2- | |
| | (propan-2-yl)phenyl]pyridine-3-carboxamide | |
| Example 393 | 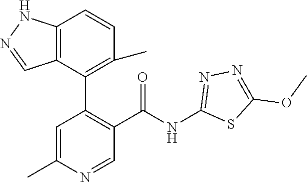 [SMILES: COc1nnc(NC(=O)c2cnc(C)cc2-c2c(C)ccc3[nH]ncc23)s1] | MS (ESI) calc'd for (C₁₈H₁₆N₆O₂S) (M + 1)⁺, 381.1, found 381.1. |
| | N-(5-methoxy-1,3,4-thiadiazol-2-yl)-6-methyl-4-(5- | |
| | methyl-1H-indazol-4-yl)nicotinamide | |
| Example 394 | 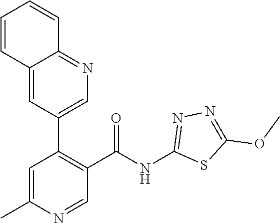 | MS (ESI) calc'd for (C₁₉H₁₅N₅O₂S) (M + 1)⁺, 378.1, found 378.0. |
| | N-(5-methoxy-1,3,4-thiadiazol-2-yl)-6-methyl-4- | |
| | (quinolin-3-yl)pyridine-3-carboxamide | |
| Example 395 | 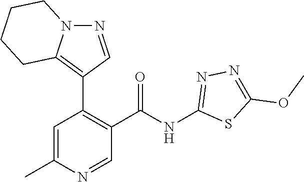 [SMILES: COc1nnc(NC(=O)c2cnc(C)cc2-c2cnn3c2CCCC3)s1] | MS (ESI) calc'd for (C₁₇H₁₈N₆O₂S) (M + 1)⁺, 371.1, found 371.1. |
| | N-(5-methoxy-1,3,4-thiadiazol-2-yl)-6-methyl-4- | |
| | {4H,5H,6H,7H-pyrazolo[1,5-a]pyridin-3-yl}pyridine-3- | |
| | carboxamide | |
| Example 396 | 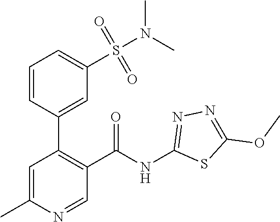 | MS (ESI) calc'd for (C₁₈H₁₉N₅O₄S₂) (M + 1)⁺, 434.1, found 434.1. |
| | 4-[3-(dimethylsulfamoyl)phenyl]-N-(5-methoxy-1,3,4- | |
| | thiadiazol-2-yl)-6-methylpyridine-3-carboxamide | |
| Example 397 | 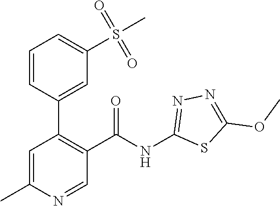 | MS (ESI) calc'd for (C₁₇H₁₆N₄O₄S₂) (M + 1)⁺, 405.1, found 405.0. |
| | 4-(3-methanesulfonylphenyl)-N-(5-methoxy-1,3,4- | |
| | thiadiazol-2-yl)-6-methylpyridine-3-carboxamide | |
| Example 398 | 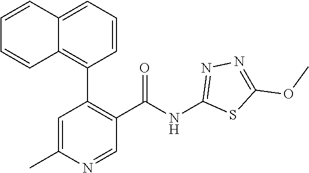 [SMILES: COc1nnc(NC(=O)c2cnc(C)cc2-c2cccc3ccccc23)s1] | MS (ESI) calc'd for (C₂₀H₁₆N₄O₂S) (M + 1)⁺, 377.1, found 377.1. |
| | N-(5-methoxy-1,3,4-thiadiazol-2-yl)-6-methyl-4- | |
| | (naphthalen-1-yl)pyridine-3-carboxamide | |
| Example 399 | 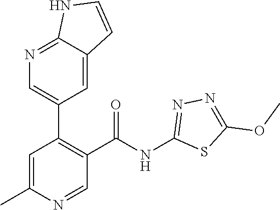 | MS (ESI) calc'd for (C₁₇H₁₄N₆O₂S) (M + 1)⁺, 367.1, found 367.0. |
| | N-(5-methoxy-1,3,4-thiadiazol-2-yl)-6-methyl-4-{1H- | |
| | pyrrolo[2,3-b]pyridin-5-yl}pyridine-3-carboxamide | |
| Example 400 | 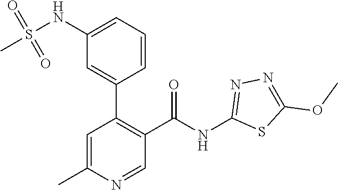 | MS (ESI) calc'd for (C₁₇H₁₇N₅O₄S₂) (M + 1)⁺, 420.1, found 420.1. |
| | 4-(3-methanesulfonamidophenyl)-N-(5-methoxy-1,3,4- | |
| | thiadiazol-2-yl)-6-methylpyridine-3-carboxamide | |
| Example 401 | 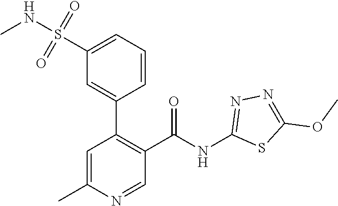 | MS (ESI) calc'd for (C₁₇H₁₇N₅O₄S₂) (M + 1)⁺, 420.1, found 420.1. |
| | N-(5-methoxy-1,3,4-thiadiazol-2-yl)-6-methyl-4-[3- | |
| | (methylsulfamoyl)phenyl]pyridine-3-carboxamide | |
| Example 402 | 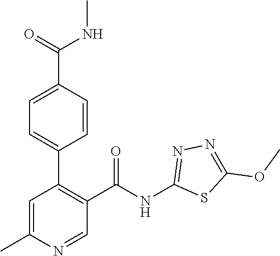 | MS (ESI) calc'd for (C₁₈H₁₇N₅O₃S) (M + 1)⁺, 384.1, found 384.1. |
| | N-(5-methoxy-1,3,4-thiadiazol-2-yl)-6-methyl-4-[4- | |
| | (methylcarbamoyl)phenyl]pyridine-3-carboxamide | |
| Example 403 | 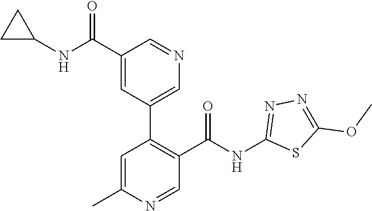 [SMILES: COc1nnc(NC(=O)c2cnc(C)cc2-c2cncc(C(=O)NC3CC3)c2)s1] | MS (ESI) calc'd for (C₁₉H₁₈N₆O₃S) (M + 1)⁺, 411.1, found 411.2. |
| | N5-cyclopropyl-N3′- | |
| | (5-methoxy-1,3,4-thiadiazol-2-yl)-6′-methyl-[3,4′- | |
| | bipyridine]-3′,5-dicarboxamide | |
| Example 404 | 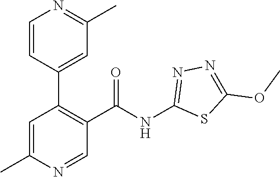 | MS (ESI) calc'd for (C₁₆H₁₅N₅O₂S) (M + 1)⁺, 342.1, found 342.0. |
| | N-(5-methoxy-1,3,4-thiadiazol-2-yl)-2′,6-dimethyl-[4,4′- | |
| | bipyridine]-3-carboxamide | |
| Example 405 | 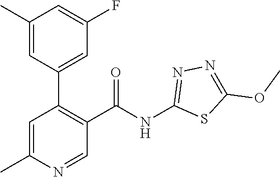 | MS (ESI) calc'd for (C₁₇H₁₅FN₄O₂S) (M + 1)⁺, 359.1, found 359.0. |
| | 4-(3-fluoro-5-methylphenyl)-N-(5-methoxy-1,3,4- | |
| | thiadiazol-2-yl)-6-methylpyridine-3-carboxamide | |
| Example 406 | 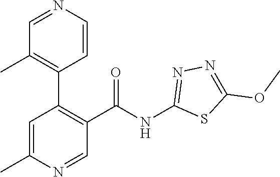 | MS (ESI) calc'd for (C₁₆H₁₅N₅O₂S) (M + 1)⁺, 342.1, found 342.0. |
| | N-(5-methoxy-1,3,4-thiadiazol-2-yl)-3′,6-dimethyl-[4,4′- | |
| | bipyridine]-3-carboxamide | |
| Example 407 | 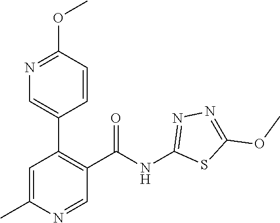 | MS (ESI) calc'd for (C₁₆H₁₅N₅O₃S) (M + 1)⁺, 358.1, found 358.0. |
| | 6-methoxy-N-(5-methoxy-1,3,4-thiadiazol-2-yl)-6′- | |
| | methyl-[3,4′-bipyridine]-3′-carboxamide | |
| Example 408 | 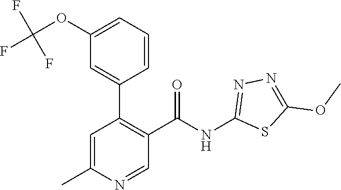 [SMILES: COc1nnc(NC(=O)c2cnc(C)cc2-c2cccc(OC(F)(F)F)c2)s1] | MS (ESI) calc'd for (C₁₇H₁₃F₃N₄O₃S) (M + 1)⁺, 411.1, found 411.1. |
| | N-(5-methoxy-1,3,4-thiadiazol-2-yl)-6-methyl-4-[3- | |
| | (trifluoromethoxy)phenyl]pyridine-3-carboxamide | |
| Example 409 | 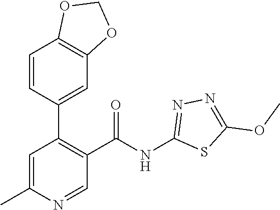 | MS (ESI) calc'd for (C₁₇H₁₄N₄O₄S) (M + 1)⁺, 371.1, found 371.1. |
| | 4-(2H-1,3-benzodioxol-5-yl)-N-(5-methoxy-1,3,4- | |
| | thiadiazol-2-yl)-6-methylpyridine-3-carboxamide | |
| Example 410 | 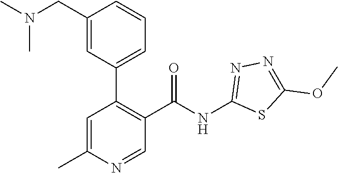 | MS (ESI) calc'd for (C₁₉H₂₁N₅O₂S) (M + 1)⁺, 384.1, found 384.2 |
| | 4-(3-((dimethylamino)methyl) phenyl)-N-(5-methoxy- | |
| | 1,3,4-thiadiazol-2-yl)-6 methylnicotinamide | |
| Example 411 | 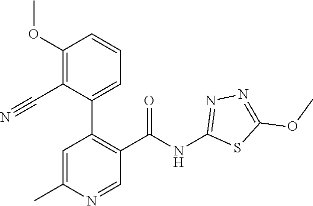 [SMILES: COc1nnc(NC(=O)c2cnc(C)cc2-c2cccc(OC)c2C#N)s1] | MS (ESI) calc'd for (C₁₈H₁₅N₅O₃S) (M + 1)⁺, 382.1, found 382.0. |
| | 4-(2-cyano-3-methoxyphenyl)-N-(5-methoxy-1,3,4- | |
| | thiadiazol-2-yl)-6-methylpyridine-3-carboxamide | |
| Example 412 | 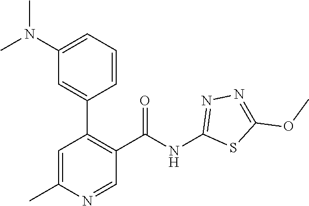 | MS (ESI) calc'd for (C₁₈H₁₉N₅O₂S) (M + 1)⁺, 370.1, found 370.1. |
| | 4-[3-(dimethylamino)phenyl]-N-(5-methoxy-1,3,4- | |
| | thiadiazol-2-yl)-6-methylpyridine-3-carboxamide | |
| Example 413 | 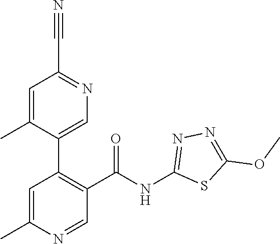 | MS (ESI) calc'd for (C₁₇H₁₄N₆O₂S) (M + 1)⁺, 367.1, found 367.1. |
| | 6-cyano-N-(5-methoxy-1,3,4-thiadiazol-2-yl)-4,6′- | |
| | dimethyl-[3,4′-bipyridine]-3′-carboxamide | |
| Example 414 | 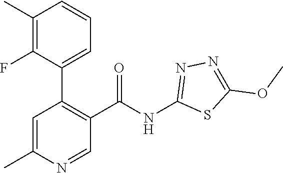 | MS (ESI) calc'd for (C₁₇H₁₅FN₄O₂S) (M + 1)⁺, 359.1, found 359.1. |
| | 4-(2-fluoro-3-methylphenyl)-N-(5-methoxy-1,3,4- | |
| | thiadiazol-2-yl)-6-methylpyridine-3-carboxamide | |
| Example 415 | 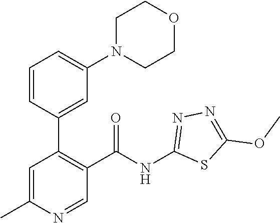 | MS (ESI) calc'd for (C₂₀H₂₁N₅O₃S) (M + 1)⁺, 412.1, found 412.2. |
| | N-(5-methoxy-1,3,4-thiadiazol-2-yl)-6-methyl-4-[3- | |
| | (morpholin-4-yl)phenyl]pyridine-3-carboxamide | |
| Example 416 | 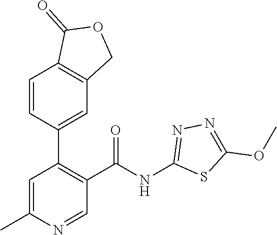 | MS (ESI) calc'd for (C₁₈H₁₄N₄O₄S) (M + 1)⁺, 383.1, found 383.1. |
| | N-(5-methoxy-1,3,4-thiadiazol-2-yl)-6-methyl-4-(1-oxo- | |
| | 1,3-dihydro-2-benzofuran-5-yl)pyridine-3-carboxamide | |
| Example 417 | 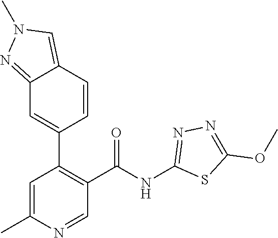 | MS (ESI) calc'd for (C₁₈H₁₆N₆O₂S) (M + 1)⁺, 381.1, found 381.1. |
| | N-(5-methoxy-1,3,4-thiadiazol-2-yl)-6-methyl-4-(2- | |
| | methyl-2H-indazol-6-yl)nicotinamide | |
| Example 418 | 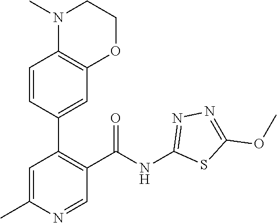 | MS (ESI) calc'd for (C₁₉H₁₉N₅O₃S) (M + 1)⁺, 398.1, found 398.2. |
| | N-(5-methoxy-1,3,4-thiadiazol-2-yl)-6-methyl-4-(4- | |
| | methyl-3,4-dihydro-2H-1,4-benzoxazin-7-yl)pyridine-3- | |
| | carboxamide | |
| Example 419 | 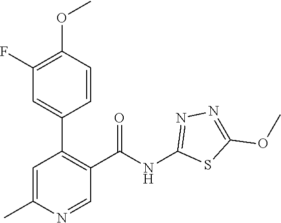 | MS (ESI) calc'd for (C₁₇H₁₅FN₄O₃S) (M + Na)⁺, 397.1, found 397.1. |
| | 4-(3-fluoro-4-methoxyphenyl)-N-(5-methoxy-1,3,4- | |
| | thiadiazol-2-yl)-6-methylpyridine-3-carboxamide | |
| Example 420 | 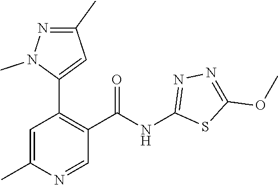 | MS (ESI) calc'd for (C₁₅H₁₆N₆O₂S) (M + 1)⁺, 345.1, found 345.2. |
| | 4-(1,3-dimethyl-1H-pyrazol-5-yl)-N-(5-methoxy-1,3,4- | |
| | thiadiazol-2-yl)-6-methylpyridine-3-carboxamide | |
| Example 421 | 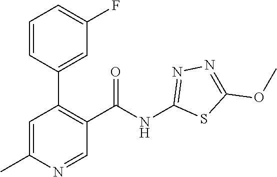 | MS (ESI) calc'd for (C₁₆H₁₃FN₄O₂S) (M + 1)⁺, 345.1, found 345.1. |
| | 4-(3-fluorophenyl)-N-(5-methoxy-1,3,4-thiadiazol-2-yl)- | |
| | 6-methylpyridine-3-carboxamide | |
| Example 422 | 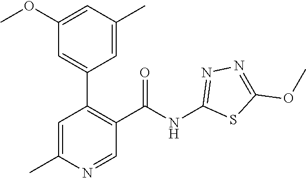 | MS (ESI) calc'd for (C₁₈H₁₈N₄O₃S) (M + 1)⁺, 371.1, found 371.2. |
| | N-(5-methoxy-1,3,4-thiadiazol-2-yl)-4-(3-methoxy-5- | |
| | methylphenyl)-6-methylpyridine-3-carboxamide | |
| Example 423 | 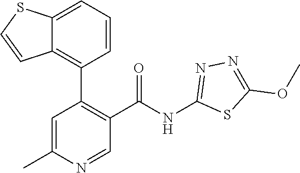 [SMILES: COc1nnc(NC(=O)c2cnc(C)cc2-c2cccc3sccc23)s1] | MS (ESI) calc'd for (C₁₈H₁₄N₄O₂S₂) (M + 1)⁺, 383.1, found 383.1. |
| | 4-(1-benzothiophen-4-yl)-N-(5-methoxy-1,3,4-thiadiazol- | |
| | 2-yl)-6-methylpyridine-3-carboxamide | |
| Example 424 | 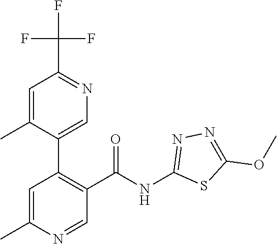 | MS (ESI) calc'd for (C₁₇H₁₄F₃N₅O₂S) (M + 1)⁺, 410.1, found 410.2. |
| | N-(5-methoxy-1,3,4-thiadiazol-2-yl)-4,6′-dimethyl-6- | |
| | (trifluoromethyl)-[3,4′-bipyridine]-3′-carboxamide | |
| Example 425 | 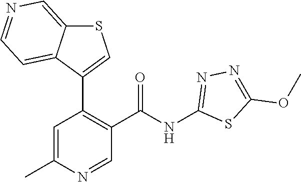 [SMILES: COc1nnc(NC(=O)c2cnc(C)cc2-c2csc3cnccc23)s1] | MS (ESI) calc'd for (C₁₇H₁₃N₅O₂S₂) (M + 1)⁺, 384.1, found 384.1. |
| | N-(5-methoxy-1,3,4-thiadiazol-2-yl)-6-methyl-4- | |
| | {thieno[2,3-c]pyridin-3-yl}pyridine-3-carboxamide | |
| Example 426 | 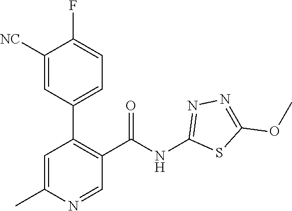 | MS (ESI) calc'd for (C₁₇H₁₂FN₅O₂S) (M + 1)⁺, 370.1, found 370.1. |
| | 4-(3-cyano-4-fluorophenyl)-N-(5-methoxy-1,3,4- | |
| | thiadiazol-2-yl)-6-methylnicotinamide | |
| Example 427 | 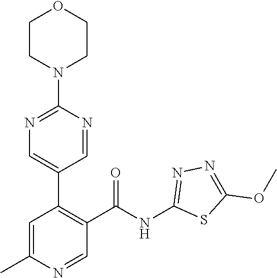 | MS (ESI) calc'd for (C₁₈H₁₉N₇O₃S) (M + 1)⁺, 414.1, found 414.2. |
| | N-(5-methoxy-1,3,4-thiadiazol-2-yl)-6-methyl-4-[2- | |
| | (morpholin-4-yl)pyrimidin-5-yl]pyridine-3-carboxamide | |
| Example 428 | 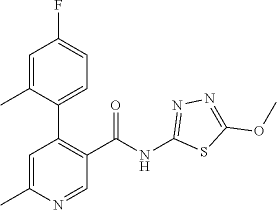 | MS (ESI) calc'd for (C₁₇H₁₅FN₄O₂S) (M + 1)⁺, 359.1, found 359.1. |
| | 4-(4-fluoro-2-methylphenyl)-N-(5-methoxy-1,3,4- | |
| | thiadiazol-2-yl)-6-methylpyridine-3-carboxamide | |
| Example 429 | 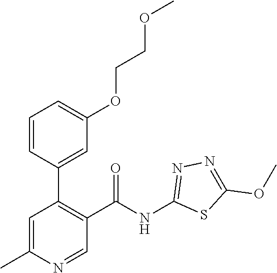 | MS (ESI) calc'd for (C₁₉H₂₀N₄O₄S) (M + 1)⁺, 401.1, found 401.2. |
| | N-(5-methoxy-1,3,4-thiadiazol-2-yl)-4-[3-(2- | |
| | methoxyethoxy)phenyl]-6-methylpyridine-3-carboxamide | |
| Example 430 | 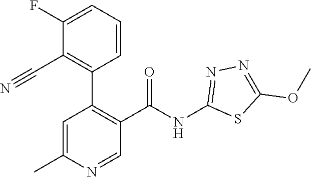 [SMILES: COc1nnc(NC(=O)c2cnc(C)cc2-c2cccc(F)c2C#N)s1] | MS (ESI) calc'd for (C₁₇H₁₂FN₅O₂S) (M + 1)⁺, 370.1, found 370.1. |
| | 4-(2-cyano-3-fluorophenyl)-N-(5-methoxy-1,3,4- | |
| | thiadiazol-2-yl)-6-methylpyridine-3-carboxamide | |
| Example 431 | 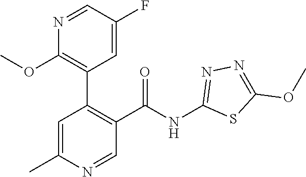 | MS (ESI) calc'd for (C₁₆H₁₄FN₅O₃S) (M + 1)⁺, 376.1, found 376.1. |
| | 5-fluoro-2-methoxy-N-(5-methoxy-1,3,4-thiadiazol-2-yl)- | |
| | 6′-methyl-[3,4′-bipyridine]-3′-carboxamide | |
| Example 432 | 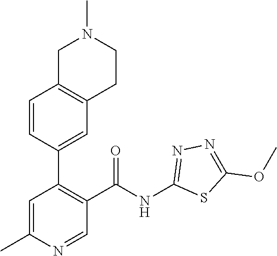 | MS (ESI) calc'd for (C₂₀H₂₁N₅O₂S) (M + 1)⁺, 396.2, found 396.2. |
| | N-(5-methoxy-1,3,4-thiadiazol-2-yl)-6-methyl-4-(2- | |
| | methyl-1,2,3,4-tetrahydroisoquinolin-6-yl)nicotinamide | |
| Example 433 | 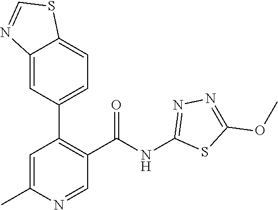 | MS (ESI) calc'd for (C₁₇H₁₃N₅O₂S₂) (M + 1)⁺, 384.1, found 384.1. |
| | 4-(benzo[d]thiazol-5-yl)-N-(5-methoxy-1,3,4-thiadiazol- | |
| | 2-yl)-6-methylnicotinamide | |
| Example 434 | 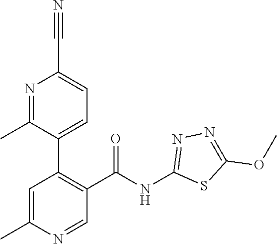 | MS (ESI) calc'd for (C₁₇H₁₄N₆O₂S) (M + 1)⁺, 367.1, found 367.1. |
| | 6-cyano-N-(5-methoxy-1,3,4-thiadiazol-2-yl)-2,6′- | |
| | dimethyl-[3,4′-bipyridine]-3′-carboxamide | |
| Example 435 | 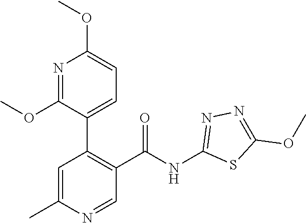 | MS (ESI) calc'd for (C₁₇H₁₇N₅O₄S) (M + 1)⁺, 388.1, found 388.1. |
| | 2,6-dimethoxy-N-(5-methoxy-1,3,4-thiadiazol-2-yl)-6′- | |
| | methyl-[3,4′-bipyridine]-3′-carboxamide | |
| Example 436 | 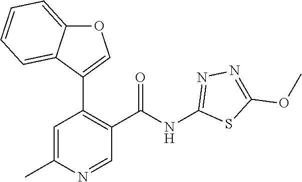 [SMILES: COc1nnc(NC(=O)c2cnc(C)cc2-c2coc3ccccc23)s1] | MS (ESI) calc'd for (C₁₈H₁₄N₄O₃S) (M + 1)⁺, 367.1, found 367.0. |
| | 4-(1-benzofuran-3-yl)-N-(5-methoxy-1,3,4-thiadiazol-2- | |
| | yl)-6-methylpyridine-3-carboxamide | |
| Example 437 | 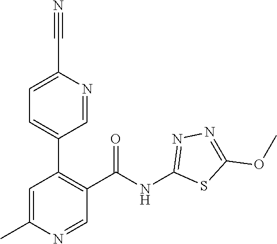 | MS (ESI) calc'd for (C₁₆H₁₂N₆O₂S) (M + 1)⁺, 353.1, found 353.0. |
| | 6-cyano-N-(5-methoxy-1,3,4-thiadiazol-2-yl)-6′-methyl- | |
| | [3,4′-bipyridine]-3′-carboxamide | |
| Example 438 | 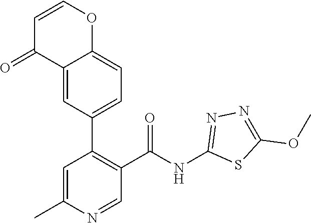 [SMILES: COc1nnc(NC(=O)c2cnc(C)cc2-c2ccc3occc(=O)c3c2)s1] | MS (ESI) calc'd for (C₁₉H₁₄N₄O₄S) (M + 1)⁺, 395.1, found 395.1. |
| | N-(5-methoxy-1,3,4-thiadiazol-2-yl)-6-methyl-4-(4-oxo- | |
| | 4H-chromen-6-yl)nicotinamide | |
| Example 439 | 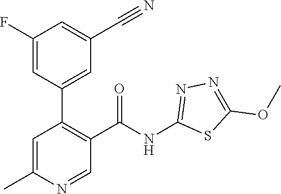 | MS (ESI) calc'd for (C₁₇H₁₂FN₅O₂S) (M + 1)⁺, 370.1, found 370.1. |
| | 4-(3-cyano-5-fluorophenyl)-N-(5-methoxy-1,3,4- | |
| | thiadiazol-2-yl)-6-methylpyridine-3-carboxamide | |
| Example 440 | 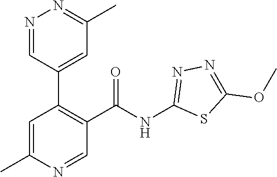 | MS (ESI) calc'd for (C₁₅H₁₄N₆O₂S) (M + 1)⁺, 343.1, found 343.1. |
| | N-(5-methoxy-1,3,4-thiadiazol-2-yl)-6-methyl-4-(6- | |
| | methylpyridazin-4-yl)nicotinamide | |
| Example 441 | 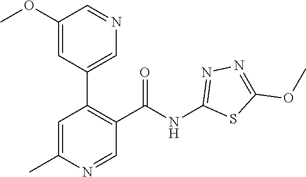 | MS (ESI) calc'd for (C₁₆H₁₅N₅O₃S) (M + 1)⁺, 358.1, found 358.1. |
| | 5-methoxy-N-(5-methoxy-1,3,4-thiadiazol-2-yl)-6′- | |
| | methyl-[3,4′-bipyridine]-3′-carboxamide | |
| Example 442 | 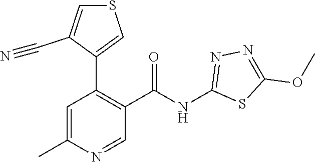 [SMILES: COc1nnc(NC(=O)c2cnc(C)cc2-c2cscc2C#N)s1] | MS (ESI) calc'd for (C₁₅H₁₁N₅O₂S₂) (M + 1)⁺ , 358.0, found 358.1. |
| | 4-(4-cyanothiophen-3-yl)-N-(5-methoxy-1,3,4-thiadiazol- | |
| | 2-yl)-6-methylpyridine-3-carboxamide | |
| Example 443 | 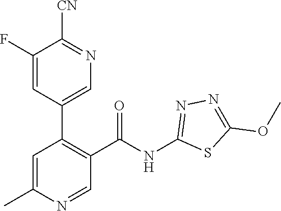 | MS (ESI) calc'd for (C₁₆H₁₁FN₆O₂S) (M + 1)⁺, 371.1, found 371.1. |
| | 6-cyano-5-fluoro-N-(5-methoxy-1,3,4-thiadiazol-2-yl)-6′- | |
| | methyl-[3,4′-bipyridine]-3′-carboxamide | |
| Example 444 | 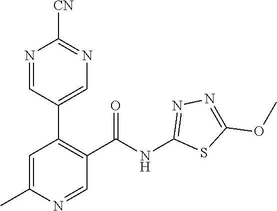 | MS (ESI) calc'd for (C₁₅H₁₁N₇O₂S) (M + 1)⁺, 354.1, found 354.1. |
| | 4-(2-cyanopyrimidin-5-yl)-N-(5-methoxy-1,3,4- | |
| | thiadiazol-2-yl)-6-methylnicotinamide | |
| Example 445 | 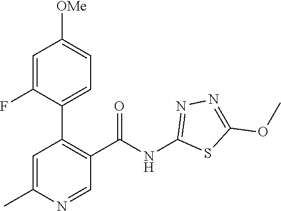 | MS (ESI) calc'd for (C₁₇H₁₅FN₄O₃S) (M + 1)⁺, 375.1, found 375.1. |
| | 4-(2-fluoro-4-methoxyphenyl)-N-(5-methoxy-1,3,4- | |
| | thiadiazol-2-yl)-6-methylnicotinamide | |
| Example 446 | 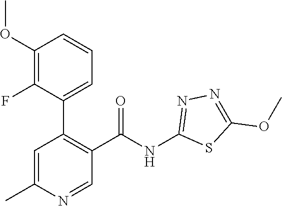 | MS (ESI) calc'd for (C₁₇H₁₅FN₄O₃S) (M + 1)⁺, 375.1, found 375.1. |
| | 4-(2-fluoro-3-methoxyphenyl)-N-(5-methoxy-1,3,4- | |
| | thiadiazol-2-yl)-6-methylpyridine-3-carboxamide | |
| Example 447 | 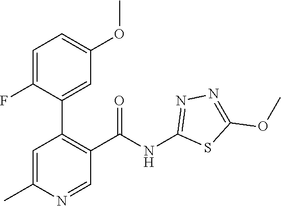 | MS (ESI) calc'd for (C₁₇H₁₅FN₄O₃S) (M + 1)⁺, 375.1, found 375.2. |
| | 4-(2-fluoro-5-methoxyphenyl)-N-(5-methoxy-1,3,4- | |
| | thiadiazol-2-yl)-6-methylpyridine-3-carboxamide | |
| Example 448 | 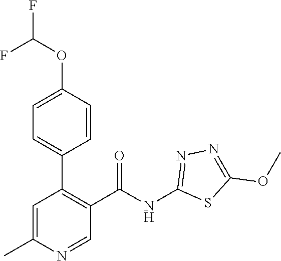 | MS (ESI) calc'd for (C₁₇H₁₄F₂N₄O₃S) (M + 1)⁺, 393.1, found 393.1. |
| | 4-[4-(difluoromethoxy)phenyl]-N-(5-methoxy-1,3,4- | |
| | thiadiazol-2-yl)-6-methylpyridine-3-carboxamide | |
| Example 449 | 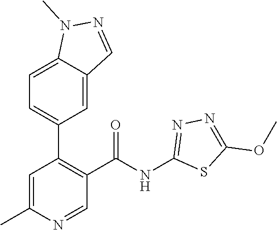 | MS (ESI) calc'd for (C₁₈H₁₆N₆O₂S) (M + 1)⁺, 381.1, found 381.2. |
| | N-(5-methoxy-1,3,4-thiadiazol-2-yl)-6-methyl-4-(1- | |
| | methyl-1H-indazol-5-yl)pyridine-3-carboxamide | |
| Example 450 | 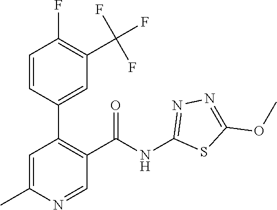 | MS (ESI) calc'd for (C₁₇H₁₂F₄N₄O₂S) (M + 1)⁺, 413.1, found 413.1. |
| | 4-[4-fluoro-3-(trifluoromethyl)phenyl]-N-(5-methoxy- | |
| | 1,3,4-thiadiazol-2-yl)-6-methylpyridine-3-carboxamide | |
| Example 451 | 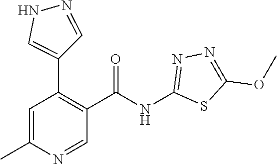 | MS (ESI) calc'd for (C₁₃H₁₂N₆O₂S) (M + 1)⁺, 317.1, found 317.2. |
| | N-(5-methoxy-1,3,4-thiadiazol-2-yl)-6-methyl-4-(1H- | |
| | pyrazol-4-yl)nicotinamide | |
| Example 452 | 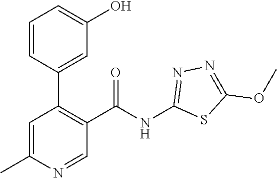 | MS (ESI) calc'd for (C₁₆H₁₄N₄O₃S) (M + 1)⁺, 343.1, found 343.1. |
| | 4-(3-hydroxyphenyl)-N-(5-methoxy-1,3,4-thiadiazol-2- | |
| | yl)-6-methylpyridine-3-carboxamide | |
| Example 453 | 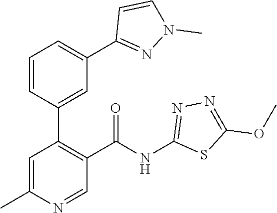 | MS (ESI) calc'd for (C₂₀H₁₈N₆O₂S) (M + 1)⁺, 407.1, found 407.2. |
| | N-(5-methoxy-1,3,4-thiadiazol-2-yl)-6-methyl-4-[3-(1- | |
| | methyl-1H-pyrazol-3-yl)phenyl]pyridine-3-carboxamide | |
| Example 454 | 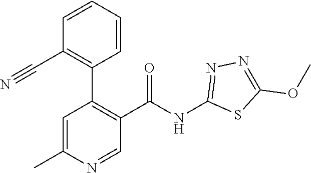 [SMILES: COc1nnc(NC(=O)c2cnc(C)cc2-c2ccccc2C#N)s1] | MS (ESI) calc'd for (C₁₇H₁₃N₅O₂S) (M + 1)⁺, 352.1, found 352.2. |
| | 4-(2-cyanophenyl)-N-(5-methoxy-1,3,4-thiadiazol-2-yl)- | |
| | 6-methylpyridine-3-carboxamide | |
| Example 455 | 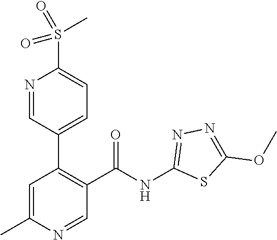 | MS (ESI) calc'd for (C₁₆H₁₅N₅O₄S₂) (M + 1)⁺, 406.1, found 406.1. |
| | N-(5-methoxy-1,3,4-thiadiazol-2-yl)-6′-methyl-6- | |
| | (methylsulfonyl)-[3,4′-bipyridine]-3′-carboxamide | |
| Example 456 | 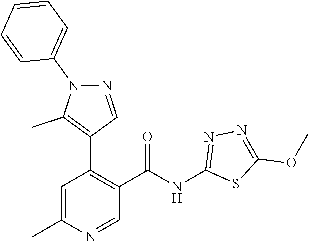 [SMILES: COc1nnc(NC(=O)c2cnc(C)cc2-c2cnn(-c3ccccc3)c2C)s1] | MS (ESI) calc'd for (C₂₀H₁₈N₆O₂S) (M + 1)⁺, 407.1, found 407.2. |
| | N-(5-methoxy-1,3,4-thiadiazol-2-yl)-6-methyl-4-(5- | |
| | methyl-1-phenyl-1H-pyrazol-4-yl)pyridine-3- | |
| | carboxamide | |
| Example 457 | 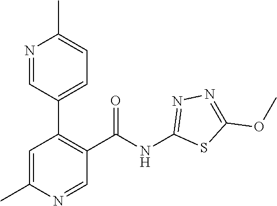 | MS (ESI) calc'd for (C₁₆H₁₅N₅O₂S) (M + 1)⁺, 342.1, found 342.15. |
| | N-(5-methoxy-1,3,4-thiadiazol-2-yl)-6,6′-dimethyl-[3,4′- | |
| | bipyridine]-3′-carboxamide | |
| Example 458 | 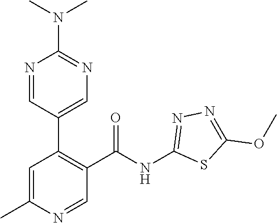 | MS (ESI) calc'd for (C₁₆H₁₇N₇O₂S) (M + 1)⁺, 372.1, found 372.1. |
| | 4-(2-(dimethylamino)pyrimidin-5-yl)-N-(5-methoxy- | |
| | 1,3,4-thiadiazol-2-yl)-6-methylnicotinamide | |
| Example 459 | 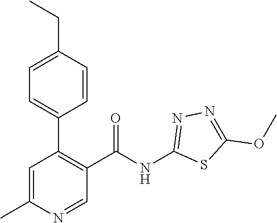 | MS (ESI) calc'd for (C₁₈H₁₈N₄O₂S) (M + 1)⁺, 355.1, found 355.2. |
| | 4-(4-ethylphenyl)-N-(5-methoxy-1,3,4-thiadiazol-2-yl)-6- | |
| | methylpyridine-3-carboxamide | |
| Example 460 | 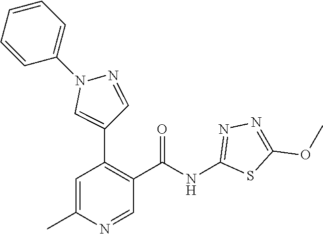 [SMILES: COc1nnc(NC(=O)c2cnc(C)cc2-c2cnn(-c3ccccc3)c2)s1] | MS (ESI) calc'd for (C₁₉H₁₆N₆O₂S) (M + 1)⁺, 393.1, found 393.2. |
| | N-(5-methoxy-1,3,4-thiadiazol-2-yl)-6-methyl-4-(1- | |
| | phenyl-1H-pyrazol-4-yl)nicotinamide | |
| Example 461 | 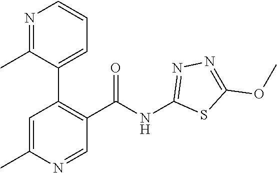 | MS (ESI) calc'd for (C₁₆H₁₅N₅O₂S) (M + 1)⁺, 342.1, found 342.2. |
| | N-(5-methoxy-1,3,4-thiadiazol-2-yl)-2,6′-dimethyl-[3,4′- | |
| | bipyridine]-3′-carboxamide | |
| Example 462 | 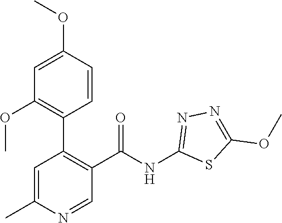 | MS (ESI) calc'd for (C₁₈H₁₈N₄O₄S) (M + 1)⁺, 387.1, found 387.2. |
| | 4-(2,4-dimethoxyphenyl)-N-(5-methoxy-1,3,4-thiadiazol- | |
| | 2-yl)-6-methylpyridine-3-carboxamide | |
| Example 463 | 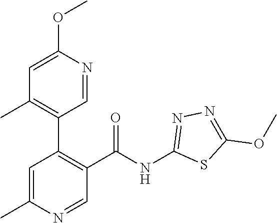 | MS (ESI) calc'd for (C₁₇H₁₇N₅O₃S) (M + 1)⁺, 372.1, found 372.1. |
| | 6-methoxy-N-(5-methoxy-1,3,4-thiadiazol-2-yl)-4,6′- | |
| | dimethyl-[3,4′-bipyridine]-3′-carboxamide | |
| Example 464 | 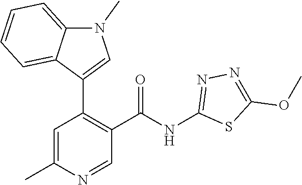 [SMILES: COc1nnc(NC(=O)c2cnc(C)cc2-c2cn(C)c3ccccc23)s1] | MS (ESI) calc'd for (C₁₉H₁₇N₅O₂S) (M + 1)⁺, 380.1, found 380.2. |
| | N-(5-methoxy-1,3,4-thiadiazol-2-yl)-6-methyl-4-(1- | |
| | methyl-1H-indol-3-yl)pyridine-3-carboxamide | |
| Example 465 | 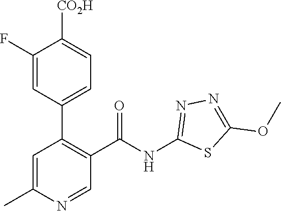 | MS (ESI) calc'd for (C₁₇H₁₃FN₄O₄S) (M + 1)⁺, 389.1, found 389.1 |
| | 2-fluoro-4-(5-((5-methoxy-1,3,4-thiadiazol-2- | |
| | yl)carbamoyl)-2-methylpyridin-4-yl)benzoic acid | |
| Example 466 | 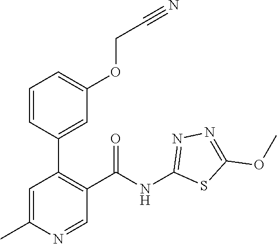 | MS (ESI) calc'd for (C₁₈H₁₅N₅O₃S) (M + 1)⁺, 382.1, found 382.1. |
| | 4-[3-(cyanomethoxy)phenyl]-N-(5-methoxy-1,3,4- | |
| | thiadiazol-2-yl)-6-methylpyridine-3-carboxamide | |
| Example 467 | 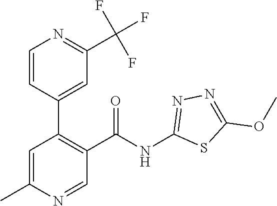 | MS (ESI) calc'd for (C₁₆H₁₂F₃N₅O₂S) (M + 1)⁺, 396.1, found 396.1. |
| | N-(5-methoxy-1,3,4-thiadiazol-2-yl)-6-methyl-2′- | |
| | (trifluoromethyl)-[4,4′-bipyridine]-3-carboxamide | |
| Example 468 | 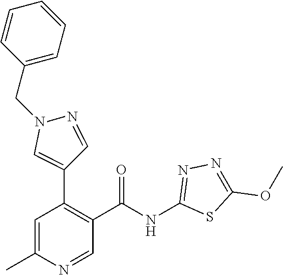 [SMILES: COc1nnc(NC(=O)c2cnc(C)cc2-c2cnn(Cc3ccccc3)c2)s1] | MS (ESI) calc'd for (C₂₀H₁₈N₆O₂S) (M + 1)⁺, 407.1, found 407.1. |
| | 4-(1-benzyl-1H-pyrazol-4-yl)-N-(5-methoxy-1,3,4- | |
| | thiadiazol-2-yl)-6-methylnicotinamide | |
| Example 469 | 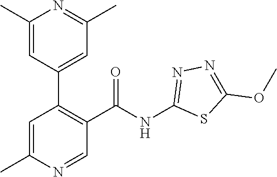 | MS (ESI) calc'd for (C₁₇H₁₇N₅O₂S) (M + 1)⁺, 356.1, found 356.2. |
| | N-(5-methoxy-1,3,4-thiadiazol-2-yl)-2′,6,6′-trimethyl- | |
| | [4,4′-bipyridine]-3-carboxamide | |
| Example 470 | 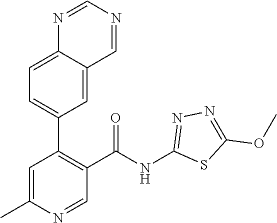 | MS (ESI) calc'd for (C₁₈H₁₄N₆O₂S) (M + Na)⁺, 401.1, found 401.1 |
| | N-(5-methoxy-1,3,4-thiadiazol-2-yl)-6-methyl-4- | |
| | (quinazolin-6-yl)pyridine-3-carboxamide | |
| Example 471 | 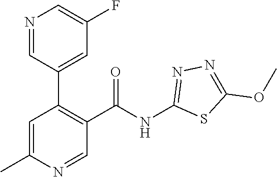 | MS (ESI) calc'd for (C₁₅H₁₂FN₅O₂S) (M + 1)⁺, 346.0, found 346.1 |
| | 5-fluoro-N-(5-methoxy-1,3,4-thiadiazol-2-yl)-6′-methyl- | |
| | [3,4′-bipyridine]-3′-carboxamide | |
| Example 472 | 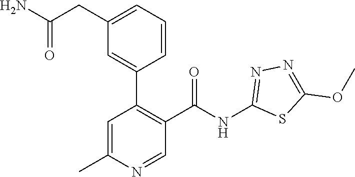 [SMILES: COc1nnc(NC(=O)c2cnc(C)cc2-c2cccc(CC(N)=O)c2)s1] | MS (ESI) calc'd for (C₁₈H₁₇N₅O₃S) (M + 1)⁺, 384.1, found 384.2 |
| | 4-[3-(carbamoylmethyl)phenyl]-N-(5-methoxy-1,3,4- | |
| | thiadiazol-2-yl)-6-methylpyridine-3-carboxamide | |
| Example 473 | 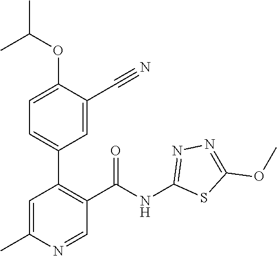 | MS (ESI) calc'd for (C₂₀H₁₉N₅O₃S) (M + 1)⁺, 410.1, found 410.2 |
| | 4-[3-cyano-4-(propan-2-yloxy)phenyl]-N-(5-methoxy- | |
| | 1,3,4-thiadiazol-2-yl)-6-methylpyridine-3-carboxamide | |
| Example 474 | 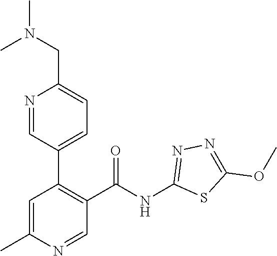 | MS (ESI) calc'd for (C₁₈H₂₀N₆O₂S) (M + 1)⁺, 384.1, found 384.2 |
| | 6-((dimethylamino)methyl)-N-(5-methoxy-1,3,4- | |
| | thiadiazol-2-yl)-6′-methyl-[3,4′-bipyridine]-3′- | |
| | carboxamide | |
| Example 475 | 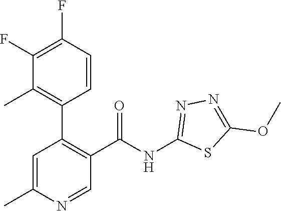 | MS (ESI) calc'd for (C₁₇H₁₄F₂N₄O₂S) (M + 1)⁺, 377.1, found 377.1 |
| | 4-(3,4-difluoro-2-methylphenyl)-N-(5-methoxy-1,3,4- | |
| | thiadiazol-2-yl)-6-methylpyridine-3-carboxamide | |
| Example 476 | 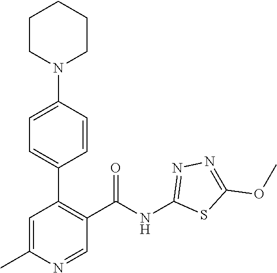 | MS (ESI) calc'd for (C₂₁H₂₃N₅O₂S) (M + 1)⁺, 410.2, found 410.2 |
| | N-(5-methoxy-1,3,4-thiadiazol-2-yl)-6-methyl-4-[4- | |
| | (piperidin-1-yl)phenyl]pyridine-3-carboxamide | |
| Example 477 | 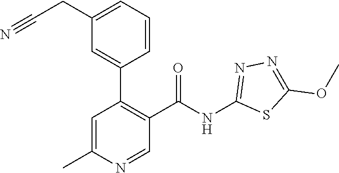 [SMILES: COc1nnc(NC(=O)c2cnc(C)cc2-c2cccc(CC#N)c2)s1] | MS (ESI) calc'd for (C₁₈H₁₅N₅O₂S) (M + 1)⁺, 366.1, found 366.2 |
| | 4-[3-(cyanomethyl)phenyl]-N-(5-methoxy-1,3,4- | |
| | thiadiazol-2-yl)-6-methylpyridine-3-carboxamide | |
| Example 478 | 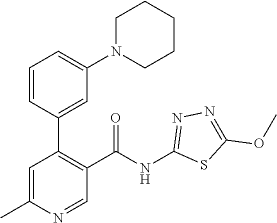 | MS (ESI) calc'd for (C₂₁H₂₃N₅O₂S) (M + 1)⁺, 410.2, found 410.2 |
| | N-(5-methoxy-1,3,4-thiadiazol-2-yl)-6-methyl-4-[3- | |
| | (piperidin-1-yl)phenyl]pyridine-3-carboxamide | |
| Example 479 | 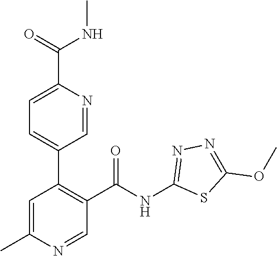 | MS (ESI) calc'd for (C₁₇H₁₆N₆O₃S) (M + 1)⁺, 385.1, found 385.1 |
| | N3′-(5-methoxy-1,3,4-thiadiazol-2-yl)-N6,6′-dimethyl- | |
| | [3,4′-bipyridine]-3′,6-dicarboxamide | |
| Example 480 | 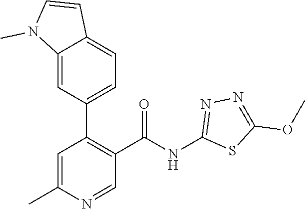 | MS (ESI) calc'd for (C₁₉H₁₇N₅O₂S) (M + 1)⁺, 380.1, found 380.2 |
| | N-(5-methoxy-1,3,4-thiadiazol-2-yl)-6-methyl-4-(1- | |
| | methyl-1H-indol-6-yl)pyridine-3-carboxamide | |
| Example 481 | 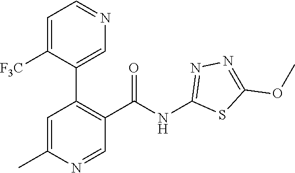 | MS (ESI) calc'd for (C₁₆H₁₂F₃N₅O₂S) (M + 1)⁺, 396.1, found 396.1 |
| | N-(5-methoxy-1,3,4-thiadiazol-2-yl)-6′-methyl-4- | |
| | (trifluoromethyl)-[3,4′-bipyridine]-3′-carboxamide | |
| Example 482 | 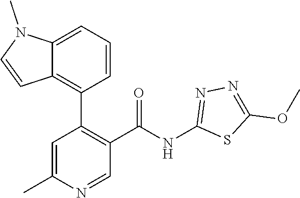 [SMILES: COc1nnc(NC(=O)c2cnc(C)cc2-c2cccc3c2ccn3C)s1] | MS (ESI) calc'd for (C₁₉H₁₇N₅O₂S) (M + 1)⁺, 380.1, found 380.2 |
| | N-(5-methoxy-1,3,4-thiadiazol-2-yl)-6-methyl-4-(1- | |
| | methyl-1H-indol-4-yl)pyridine-3-carboxamide | |
| Example 483 | 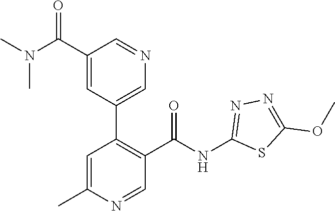 | MS (ESI) calc'd for (C₁₈H₁₈N₆O₃S) (M + 1)⁺, 399.1, found 399.1 |
| | N3′-(5-methoxy-1,3,4-thiadiazol-2-yl)-N5,N5,6′- | |
| | trimethyl-[3,4′-bipyridine]-3′,5-dicarboxamide | |
| Example 484 | 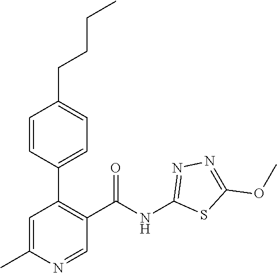 | MS (ESI) calc'd for (C₂₀H₂₂N₄O₂S) (M + Na)⁺, 405.2, found 405.2 |
| | 4-(4-butylphenyl)-N-(5-methoxy-1,3,4-thiadiazol-2-yl)-6- | |
| | methylpyridine-3-carboxamide | |
| Example 485 | 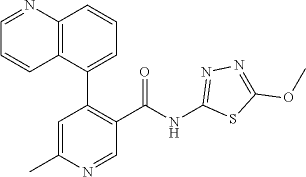 [SMILES: COc1nnc(NC(=O)c2cnc(C)cc2-c2cccc3ncccc23)s1] | MS (ESI) calc'd for (C₁₉H₁₅N₅O₂S) (M + 1)⁺, 378.1, found 378.0 |
| | N-(5-methoxy-1,3,4-thiadiazol-2-yl)-6-methyl-4- | |
| | (quinolin-5-yl)nicotinamide | |
| Example 486 | 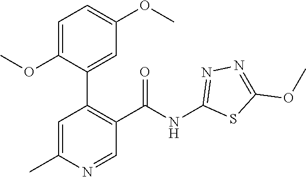 | MS (ESI) calc'd for (C₁₈H₁₈N₄O₄S) (M + 1)⁺, 387.1, found 387.1 |
| | 4-(2,5-dimethoxyphenyl)-N-(5-methoxy-1,3,4-thiadiazol- | |
| | 2-yl)-6-methylnicotinamide | |
| Example 487 | 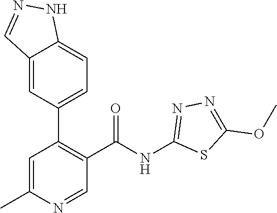 | MS (ESI) calc'd for (C₁₇H₁₄N₆O₂S) (M + 1)⁺, 367.1, found 367.0 |
| | 4-(1H-indazol-5-yl)-N-(5-methoxy-1,3,4-thiadiazol-2-yl)- | |
| | 6-methylnicotin amide | |
| Example 488 | 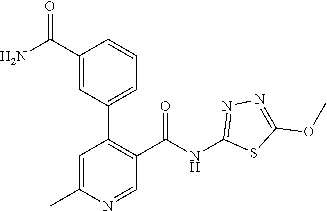 [SMILES: COc1nnc(NC(=O)c2cnc(C)cc2-c2cccc(C(N)=O)c2)s1] | MS (ESI) calc'd for (C₁₇H₁₅N₅O₃S) (M + 1)⁺, 370.1, found 370.1 |
| | 4-(3-carbamoylphenyl)-N-(5-methoxy-1,3,4-thiadiazol-2- | |
| | yl)-6-methylnicotinamide | |
| Example 489 | 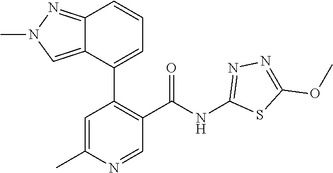 | MS (ESI) calc'd for (C₁₈H₁₆N₆O₂S) (M + 1)⁺, 381.1, found 381.1 |
| | N-(5-methoxy-1,3,4-thiadiazol-2-yl)-6-methyl-4-(2- | |
| | methyl-2H-indazol-4-yl)pyridine-3-carboxamide | |
| Example 490 | 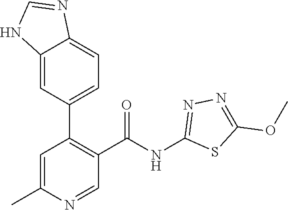 | MS (ESI) calc'd for (C₁₇H₁₄N₆O₂S) (M + 1)⁺, 367.1, found 367.1 |
| | 4-(1H-1,3-benzodiazol-6-yl)-N-(5-methoxy-1,3,4- | |
| | thiadiazol-2-yl)-6-methylpyridine-3-carboxamide | |

### Biological Assay

The ability of the compounds of Formula (I) or Table 1 to inhibit ATPase activity of Pol theta (1-899) was determined using the assay described below.

Pol Theta ATPase activity was determined by measuring the rate of ATP turn over in a NADH oxidation-coupled enzymatic assay. 10-point dilution series of compounds were used in a 384 well format for the inhibition assays. Pol theta (1-899) (5 nM) in assay buffer (20 mM Tris HCl (pH 7.80), 80 mM KCl, 10 mM MgCl₂, 1 mM DTT, 0.01% BSA, 0.01% Tween, 5% glycerol) was transferred to the test wells (20 μL), except the low control wells (20 μL of assay buffer was added to the low control wells). The plate was then incubated at room temperature for 15 min. An equal volume (20 μL) of 100 μM ATP, 300 nM dT₅₀ (single-stranded DNA (ssDNA) containing 50 thymine bases), 300 μM NADH, 6 mM PEP, 10 U/mL lactate dehydrogenase and 20 U/mL pyruvate kinase in assay buffer was added to all the test wells. The plate was then centrifuged at 1000 rpm for 1 min. The reaction was monitored for 60 min by measuring absorbance (λ=340 nm) in a Tecan Spark multimode plate reader every minute. The high control (DMSO with enzyme) with low absorbance intensity represents no inhibition of ATPase reaction while the low control (DMSO with buffer) with high absorbance intensity represents full inhibition of ATPase activity. Slope of the reaction progress curves were used to calculate the rate of ATP hydrolysis. The rates were used to determine the percent inhibition using a four-parameter inhibition model to generate IC₅₀, Hill slope and max inhibition.

The IC₅₀ of the compounds in Table 1 above are disclosed in Table 4, below:

| | ATPase NADH |
|---|---|
| | coupling:Standard |
| | Screening:hPolQ |
| Cpd # | IC50 |
| 1 | ++++ |
| 2 | ++++ |
| 3 | + |
| 4 | +++ |
| 5 | ++++ |
| 6 | ++++ |
| 7 | ++++ |
| 8 | ++++ |
| 9 | ++++ |
| 10 | ++++ |
| 11 | ++++ |
| 12 | ++++ |
| 13 | ++++ |
| 14 | ++++ |
| 15 | ++++ |
| 16 | ++++ |
| 17 | ++++ |
| 18 | ++++ |
| 19 | ++++ |
| 20 | ++++ |
| 21 | ++++ |
| 22 | ++++ |
| 23 | ++++ |
| 24 | ++++ |
| 25 | ++++ |
| 26 | ++++ |
| 27 | ++++ |
| 28 | ++++ |
| 29 | ++++ |
| 30 | ++++ |
| 31 | +++ |
| 32 | ++++ |
| 33 | ++++ |
| 34 | ++ |
| 35 | +++ |
| 36 | ++++ |
| 37 | ++++ |
| 38 | ++++ |
| 39 | ++++ |
| 40 | + |
| 41 | ++++ |
| 42 | +++ |
| 43 | +++ |
| 44 | ++++ |
| 45 | ++++ |
| 46 | ++++ |
| 47 | ++++ |
| 48 | ++++ |
| 49 | ++++ |
| 50 | + |
| 51 | ++++ |
| 52 | ++++ |
| 53 | +++ |
| 54 | ++ |
| 55 | +++ |
| 56 | +++ |
| 57 | ++++ |
| 58 | ++++ |
| 59 | ++++ |
| 60 | ++++ |
| 61 | ++++ |
| 62 | ++++ |
| 63 | ++++ |
| 64 | ++++ |
| 65 | ++ |
| 66 | +++ |
| 67 | + |
| 68 | + |
| 69 | + |
| 70 | ++++ |
| 71 | + |
| 72 | ++ |
| 73 | ++ |
| 74 | ++++ |
| 75 | ++++ |
| 76 | ++++ |
| 77 | ++++ |
| 78 | ++ |

IC₅₀: 10 uM ≥ (+) > 1 uM; 1 uM ≥ (++) > 500 nM; 500 nM ≥ (+++) > 200 nM; 200 nM ≥ (++++)

The IC₅₀ of the compounds in Table 2 above are disclosed in Table 5, below:

| | ATPase NADH |
|---|---|
| | coupling:Standard |
| | Screening:hPolQ |
| Cpd # | IC50 |
| 79 | +++ |
| 80 | ++++ |
| 81 | ++++ |
| 82 | ++++ |
| 83 | ++++ |
| 84 | ++++ |
| 85 | + |
| 88 | + |
| 91 | + |
| 92 | +++ |
| 93 | ++++ |
| 98 | >10 uM |
| 99 | >10 uM |
| 100 | +++ |
| 101 | + |
| 102 | +++ |
| 103 | ++++ |
| 104 | +++ |
| 105 | + |
| 107 | >10 uM |
| 109 | + |
| 110 | >10 uM |
| 112 | +++ |
| 114 | ++++ |
| 115 | ++++ |
| 116 | ++++ |
| 117 | ++++ |
| 118 | ++++ |
| 119 | ++++ |
| 120 | >10 uM |
| 121 | >10 uM |
| 122 | ++++ |
| 123 | >10 uM |
| 124 | ++++ |
| 125 | ++++ |
| 126 | + |
| 127 | ++++ |
| 128 | ++++ |
| 129 | ++++ |
| 130 | ++++ |
| 131 | ++++ |
| 132 | ++++ |
| 133 | ++++ |
| 134 | ++ |
| 135 | ++++ |
| 136 | ++++ |
| 137 | ++++ |
| 138 | ++++ |
| 139 | +++ |
| 140 | ++++ |
| 141 | ++++ |
| 142 | ++++ |
| 143 | +++ |
| 144 | >10 uM |
| 145 | ++++ |
| 146 | ++++ |
| 147 | ++++ |
| 148 | ++++ |
| 149 | ++++ |
| 150 | ++++ |
| 151 | ++++ |
| 152 | ++++ |
| 153 | ++++ |
| 154 | ++++ |
| 155 | ++++ |
| 156 | ++++ |
| 157 | ++++ |
| 158 | ++++ |
| 159 | ++++ |
| 160 | ++++ |
| 161 | ++++ |
| 162 | ++++ |
| 163 | ++++ |
| 164 | ++++ |
| 165 | ++++ |
| 166 | ++++ |
| 167 | ++++ |
| 168 | ++++ |
| 169 | ++++ |
| 170 | ++++ |
| 171 | ++++ |
| 172 | ++++ |
| 173 | ++++ |
| 174 | ++++ |
| 175 | ++++ |
| 176 | ++++ |
| 177 | +++ |
| 178 | ++++ |
| 179 | ++++ |
| 180 | +++ |
| 181 | ++++ |
| 182 | ++++ |
| 183 | ++++ |
| 184 | ++++ |
| 185 | ++++ |
| 186 | ++++ |
| 187 | ++++ |
| 188 | ++++ |
| 189 | ++++ |
| 190 | ++++ |
| 191 | ++++ |
| 192 | ++++ |
| 193 | ++ |
| 194 | ++++ |
| 195 | ++++ |
| 196 | ++ |
| 197 | ++++ |
| 198 | ++++ |
| 199 | ++++ |
| 200 | ++++ |
| 201 | ++++ |
| 202 | ++++ |
| 203 | ++++ |
| 204 | ++++ |
| 205 | ++++ |
| 206 | ++++ |
| 207 | ++++ |
| 208 | ++++ |
| 209 | ++++ |
| 210 | ++++ |
| 211 | ++++ |
| 212 | ++++ |
| 213 | ++++ |
| 214 | ++++ |
| 215 | ++++ |
| 216 | ++++ |
| 217 | ++++ |
| 218 | ++++ |
| 219 | ++++ |
| 220 | ++++ |
| 221 | ++++ |
| 222 | ++++ |
| 223 | ++++ |
| 224 | ++++ |
| 225 | ++++ |
| 226 | ++++ |
| 227 | ++++ |
| 228 | ++++ |
| 229 | ++++ |
| 230 | ++++ |
| 231 | ++++ |
| 232 | ++++ |
| 233 | ++++ |
| 234 | >10 uM |
| 235 | >10 uM |

IC₅₀: 10 uM ≥ (+) > 1 uM; 1 uM ≥ (++) > 500 nM; 500 nM ≥ (+++) > 200 nM; 200 nM ≥ (++++)

The IC₅₀ of the compounds in Table 3 above are disclosed in Table 6, below:

| | ATPase NADH |
|---|---|
| | coupling:Standard |
| | Screening:hPolQ |
| Cpd # | IC50 |
| 236 | ++++ |
| 237 | ++++ |
| 238 | ++++ |
| 239 | ++++ |
| 240 | ++++ |
| 241 | ++++ |
| 242 | ++++ |
| 243 | ++++ |
| 244 | ++++ |
| 245 | ++++ |
| 246 | ++++ |
| 247 | ++++ |
| 248 | ++++ |
| 249 | ++++ |
| 250 | ++++ |
| 251 | ++++ |
| 252 | ++++ |
| 253 | ++++ |
| 254 | ++++ |
| 255 | ++++ |
| 256 | ++++ |
| 257 | ++++ |
| 258 | ++++ |
| 259 | ++++ |
| 260 | ++++ |
| 261 | ++++ |
| 262 | ++++ |
| 263 | ++++ |
| 264 | ++++ |
| 265 | ++++ |
| 266 | ++++ |
| 267 | ++++ |
| 268 | ++++ |
| 269 | ++++ |
| 270 | +++ |
| 271 | ++++ |
| 272 | ++++ |
| 273 | +++ |
| 274 | ++++ |
| 275 | ++++ |
| 276 | ++++ |
| 277 | ++++ |
| 278 | +++ |
| 279 | ++++ |
| 280 | ++++ |
| 281 | ++++ |
| 282 | ++++ |
| 283 | +++ |
| 284 | ++++ |
| 285 | + |
| 286 | ++++ |
| 287 | ++++ |
| 288 | ++++ |
| 289 | + |
| 290 | +++ |
| 291 | ++++ |
| 292 | ++++ |
| 293 | ++++ |
| 294 | +++ |
| 295 | ++++ |
| 296 | ++++ |
| 297 | ++++ |
| 298 | ++ |
| 299 | ++++ |
| 300 | ++++ |
| 301 | ++++ |
| 302 | ++++ |
| 303 | + |
| 304 | ++++ |
| 305 | ++++ |
| 306 | + |
| 307 | +++ |
| 308 | + |
| 309 | ++++ |
| 310 | ++++ |
| 311 | +++ |
| 312 | ++ |
| 313 | ++++ |
| 314 | ++++ |
| 315 | ++++ |
| 316 | ++++ |
| 317 | ++++ |
| 318 | ++++ |
| 319 | ++++ |
| 320 | ++++ |
| 321 | + |
| 322 | ++ |
| 323 | ++++ |
| 324 | ++++ |
| 325 | + |
| 326 | + |
| 327 | + |
| 328 | ND |
| 329 | + |
| 330 | + |
| 331 | + |
| 332 | + |
| 333 | + |
| 334 | + |
| 335 | ++ |
| 336 | + |
| 337 | ++ |
| 338 | + |
| 339 | + |
| 340 | + |
| 341 | ++ |
| 342 | + |
| 343 | + |
| 344 | + |
| 345 | + |
| 346 | + |
| 347 | + |
| 348 | ND |
| 349 | + |
| 350 | + |
| 351 | + |
| 352 | + |
| 353 | + |
| 354 | + |
| 355 | + |
| 356 | + |
| 357 | + |
| 358 | ++ |
| 359 | ++++ |
| 360 | + |
| 361 | ++ |
| 362 | + |
| 363 | ++ |
| 364 | + |
| 365 | + |
| 366 | + |
| 367 | +++ |
| 368 | + |
| 369 | + |
| 370 | + |
| 371 | + |
| 372 | +++ |
| 373 | ND |
| 374 | ND |
| 375 | ND |
| 376 | + |
| 377 | + |
| 378 | + |
| 379 | + |
| 380 | + |
| 381 | + |
| 382 | + |
| 383 | ++ |
| 384 | + |
| 385 | + |
| 386 | ++ |
| 387 | + |
| 388 | + |
| 389 | ++ |
| 390 | + |
| 391 | + |
| 392 | + |
| 393 | ND |
| 394 | + |
| 395 | + |
| 396 | + |
| 397 | ++ |
| 398 | ++++ |
| 399 | + |
| 400 | + |
| 401 | + |
| 402 | + |
| 403 | + |
| 404 | + |
| 405 | +++ |
| 406 | + |
| 407 | + |
| 408 | ++ |
| 409 | ++ |
| 410 | + |
| 411 | + |
| 412 | + |
| 413 | + |
| 414 | ++++ |
| 415 | + |
| 416 | + |
| 417 | + |
| 418 | + |
| 419 | + |
| 420 | + |
| 421 | ++ |
| 422 | ++ |
| 423 | +++ |
| 424 | + |
| 425 | ++ |
| 426 | ND |
| 427 | + |
| 428 | ++ |
| 429 | + |
| 430 | ++ |
| 431 | ++ |
| 432 | + |
| 433 | ND |
| 434 | + |
| 435 | +++ |
| 436 | + |
| 437 | + |
| 438 | + |
| 439 | ++ |
| 440 | + |
| 441 | ND |
| 442 | + |
| 443 | ND |
| 444 | + |
| 445 | + |
| 446 | + |
| 447 | ++ |
| 448 | + |
| 449 | ++ |
| 450 | ++++ |
| 451 | + |
| 452 | + |
| 453 | + |
| 454 | + |
| 455 | + |
| 456 | + |
| 458 | + |
| 458 | + |
| 459 | + |
| 460 | + |
| 461 | + |
| 462 | +++ |
| 463 | + |
| 464 | + |
| 465 | + |
| 466 | + |
| 467 | ++ |
| 468 | + |
| 469 | +++ |
| 470 | + |
| 471 | + |
| 472 | + |
| 473 | ++++ |
| 474 | + |
| 475 | ++ |
| 476 | + |
| 477 | + |
| 478 | + |
| 479 | + |
| 480 | + |
| 481 | + |
| 482 | + |
| 483 | + |
| 484 | + |
| 485 | +++ |
| 486 | ++ |
| 487 | ++ |
| 488 | + |
| 489 | + |
| 490 | + |

* ND means “not determined.”
IC₅₀: 10 uM ≥ (+) > 1 uM; 1 uM ≥ (++) > 500 nM; 500 nM ≥ (+++) > 200 nM; 200 nM ≥ (++++)

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

What is claimed is:

1. A compound of Formula (I): 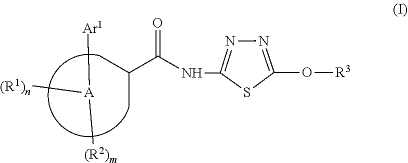
wherein:
ring A is selected from the group consisting of phenyl and a 5- to 6-membered heteroaryl ring having 1 to 4 heteroatoms independently selected from N, O, and S;
the subscripts m and n are each independently 0 or 1;
R¹ is selected from the group consisting of C₁₋₆ alkyl, halo, C₁₋₆ haloalkyl, —X¹—O—C₁₋₆ alkyl,
C₁₋₆ haloalkoxy, —X¹-cyano, —NO₂, —C(O)OR^{a}, —NR^{a}C(O)R^{b}, —X¹—C(O)NR^{a}R^{b}, —X¹—OH, C₃₋₆ cycloalkyl, —X¹—O—C₃₋₆ cycloalkyl, C₁₋₆ hydroxyalkynyl, —X¹—NR^{a}R^{b}, —X¹—S(O)₂R^{a}, —X¹—S(O)₂NR^{a}R^{b}, X¹—X^{1a}—OR^{a}, and a 4- to 6-member heterocycloalkyl having 1 to 4 heteroatoms as ring vertices independently selected from N, O, and S;
R² is selected from the group consisting of C₁₋₆ alkyl, halo, C₁₋₆ haloalkyl, C₁₋₆ haloalkoxy, and
—X¹-cyano, wherein
each X¹ is independently selected from a bond and C₁₋₄ alkylene,
X^{1a} is 3- to 6-membered heterocycloalkylene having 1 to 4 heteroatoms as ring vertices independently selected from N, O, and S, and
R^{a} and R^{b} are independently selected from the group consisting of hydrogen, C₁₋₆ alkyl, and C₁₋₆ haloalkyl; and
Ar¹ is selected form the group consisting of phenyl, naphthyl, pyridin-2-one, and a 5- to 10-membered heteroaryl ring having 1 to 4 heteroatoms independently selected from N, O, and S, wherein Ar¹ is substituted with 0 to 4 R^{1a} substituents;
each R^{1a} is independently selected from C₁₋₆ alkyl, halo, C₁₋₆ haloalkyl, —X²—O—C₁₋₆ alkyl, C₁₋₆ haloalkoxy, C₃₋₆ cycloalkyl, C₃₋₆ cycloalkyloxy, —C(O)R^{c}, —C(O)₂R^{c}, —NR^{c}C(O)R^{d}, —O—C₁₋₄ alkylene-O—C₁₋₄ alkyl, —X²—C(O)NR^{c}R^{d}, —X²—S(O)₂NR^{c}R^{d}, —X²—NR^{c}R^{d}, —C(O)NR^{c}R^{d}, —X²-cyano, —O—X²-cyano, —X²—S(O)R^{c}, —X²—S(O)₂R^{c}, —X²—N(R^{d})S(O)₂R^{c}, —P(O)R^{c}R^{d}, —Y and —X²—OH; or
two R^{1a} groups on adjacent ring vertices combine to form a 4 to 6 membered cycloalkyl or heterocycloalkyl, having 0 to 2 heteroatoms as ring vertices independently selected from N, O, and S, and which is substituted with 0 to 4 groups independently selected from oxo, halo, C₁₋₄ alkyl, C₁₋₄ alkoxy, and C₁₋₄ haloalkyl;
wherein
each Y is independently selected from phenyl, benzyl, 4- to 6-membered heterocycloalkyl, and 5- or 6-membered heteroaryl, wherein each heterocycloalkyl and heteroaryl has 1 or 2 ring members independently selected from O, N and S; and each Y is substituted with 0, 1 or 2 groups independently selected from halo, oxo, C₁₋₄ alkyl, C₁₋₄ alkoxy, and C₁₋₄ haloalkyl;
each X² is independently selected from a bond and C₁₋₄ alkylene; and
each R^{c} and R^{d} are independently selected from the group consisting of hydrogen, C₁₋₆ alkyl, C₃₋₅ cycloalkyl and C₁₋₆ haloalkyl; and
R³ is a member selected from the group consisting of
(i) C₃₋₆ cycloalkyl, C₆₋₁₁ bridged cycloalkyl and C₆₋₁₂ spirocycloalkyl;
(ii) 3- to 6-membered heterocycloalkyl having 1 to 4 heteroatoms as ring vertices independently selected from N, O, and S;
(iii) 6- to 10-membered bicyclic heterocyclyl having 1 to 4 heteroatoms as ring vertices independently selected from N, O, and S;
(iv) 6- to 10-membered bridged heterocyclyl having 1 to 4 heteroatoms as ring vertices independently selected from N, O, and S;
(v) 6- to 12-membered spiroheterocyclyl having 1 to 4 heteroatoms as ring vertices independently selected from N, O, and S;
(vi) hydrogen;
(vii) C₁₋₆ alkyl, or C₂₋₆ alkynyl,
wherein
each R³ member of (i) through (v) is substituted with 0 to 4 R^{3a} substituents, each of which is independently selected from C₁₋₆ alkyl, C₂₋₆ alkenyl, C₂₋₆ alkynyl, halo, C₁₋₆ haloalkyl, C₁₋₆ haloalkoxy, —X³—O—C₁₋₆ alkyl, —X³—OH, —NR^{e}R^{f}, —ONO₂, 4- to 6-member heterocycloalkyl having 1 to 4 heteroatoms as ring vertices independently selected from N, O, and S, —NR^{e}C(O)R^{f}, —X³—NR^{e}R^{f}, —X³-cyano, and oxo; and
R³ member (vii) is substituted with from 0 to 3 R^{3b} substituents selected from the group consisting of halo, C₁₋₃ haloalkyl, C₁₋₆ haloalkoxy, —O—C₁₋₆ alkyl, cyano, —OH, —NR^{e}R^{f},
—CONR^{e}R^{f}, and oxo, wherein
each X³ is independently selected from a bond and C₁₋₄ alkylene, and
each R^{e} and R^{f} is independently selected from H, C₁₋₆ alkyl, C₁₋₆ haloalkyl, C₃₋₆ cycloalkyl and —C₁₋₃alkylene-C₃₋₆ cycloalkyl;
or a pharmaceutically acceptable salt thereof.

2-3. (canceled)

4. The compound of claim 1, or a pharmaceutically acceptable salt thereof, wherein ring A is selected from the group consisting of phenyl, pyridinyl, pyrimidinyl, imidazo[1,2-a]pyridinyl, 1,2,3-triazole, pyrazolyl, isoxazolyl, and imidazo[1,5-a]pyridinyl.

5. (canceled)

6. The compound of claim 1, or a pharmaceutically acceptable salt thereof, wherein ring A is pyridinyl.

7-8. (canceled)

9. The compound of claim 1, or a pharmaceutically acceptable salt thereof, wherein n is 1 and R¹ is selected from the group consisting of C₁₋₆ alkyl, halo, C₁₋₆ haloalkyl, —X¹—O—C₁₋₆ alkyl, C₁₋₆ haloalkoxy, —X¹-cyano, —X¹—OH, C₃₋₆ cycloalkyl, —X¹—NR^{a}R^{b}.

10-11. (canceled)

12. The compound of claim 1, or a pharmaceutically acceptable salt thereof, wherein n is 1 and R¹ is methyl.

13. The compound of claim 1, or a pharmaceutically acceptable salt thereof, wherein m is 0.

14-16. (canceled)

17. The compound of claim 1, or a pharmaceutically acceptable salt thereof, wherein Ar¹ is selected from the group consisting of phenyl, pyridinyl, benzopyrazolyl, benzimidazolyl, imidazolyl, pyridazyl, imidazo[1,2-a]pyrimidinyl, oxazolo[4,5-b]pyridinyl, oxazolo[5,4-b]pyridinyl, thiazolo[4,5-b]pyridinyl, benzo[d]thiazole, indazolyl, [1,2,4]triazolo[1,5-a]pyrimidinyl, [1,2,4]triazolo[1,5-b]pyridazinyl, and tetrazolo[1,5-a]pyridinyl, each of which is substituted with 0 to 4 R^{1a}.

18. (canceled)

19. The compound of claim 1, or a pharmaceutically acceptable salt thereof, wherein Ar¹ is pyridinyl substituted with 0 to 4 R^{1a}.

20-22. (canceled)

23. The compound of claim 1, or a pharmaceutically acceptable salt thereof, having Formula (Ia2), (Ib2), or (Ic2):

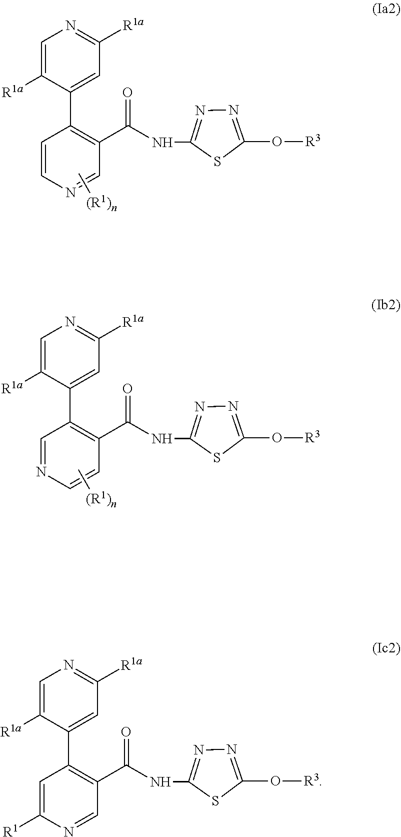

24. (canceled)

25. The compound of claim 1, or a pharmaceutically acceptable salt thereof, wherein each R^{1a} is independently selected from C₁₋₆ alkyl, halo, C₁₋₆ haloalkyl, —X²—O—C₁₋₆ alkyl, C₁₋₆ haloalkoxy, C₃₋₆ cycloalkyl, —NR^{a}R^{b}, —X²-cyano, and —X²—OH.

26-27. (canceled)

28. The compound of claim 1, or a pharmaceutically acceptable salt thereof, wherein each R^{1a} is independently selected from methyl, ethyl, fluoro, chloro, bromo, trifluoromethyl, difluoromethyl, methoxy, ethoxy, difluoromethoxy, cyclopropyl, —NH₂, hydroxymethyl, and 1-hydroxyethyl.

29-34. (canceled)

35. The compound of claim 1, or a pharmaceutically acceptable salt thereof, wherein R³ is C₁₋₆ alkyl or C₂₋₆ alkynyl, and is substituted with 0 to 4 R^{3b}.

36-42. (canceled)

43. The compound of claim 1, or a pharmaceutically acceptable salt thereof, wherein R³ is C₆₋₁₂ spirocyclyl or C₃₋₆ cycloalkyl, each of which is substituted with 0 to 4 R^{3a}.

44-45. (canceled)

46. The compound of claim 1, or a pharmaceutically acceptable salt thereof, wherein R³ is a 3- to 6-membered heterocycloalkyl having 1 to 4 heteroatoms as ring vertices independently selected from N, O, and S, and is substituted with 0 to 4 R^{3a}.

47-50. (canceled)

51. The compound of claim 1, or a pharmaceutically acceptable salt thereof, wherein R³ is a 6- to 10-membered bicyclic heterocyclyl having 1 to 4 heteroatoms as ring vertices independently selected from N, O, and S, and is substituted with 0 to 4 R^{3a}.

52-59. (canceled)

60. A compound selected from those in Table 1 or a pharmaceutically acceptable salt thereof, in Table 2 or a pharmaceutically acceptable salt thereof, and Table 3 or a pharmaceutically acceptable salt thereof.

61. A pharmaceutical composition comprising a compound of claim 1, and at least one pharmaceutically acceptable excipient.

62. A method for treating a disease characterized by overexpression of Polθ in a patient in need thereof comprising administering to the patient a therapeutically effective amount of a compound of claim 1, or a pharmaceutically acceptable salt thereof, wherein the disease is a cancer.

63. (canceled)

64. A method of treating a homologous recombinant (HR) deficient cancer in a patient in need thereof comprising administering to the patient a therapeutically effective amount of a compound of claim 1, or a pharmaceutically acceptable salt thereof.

65. (canceled)

66. A method for treating a cancer in a patient in need thereof, wherein the cancer is characterized by a reduction or absence of BRCA gene expression, the absence of the BRCA gene, or reduced function of BRCA protein, comprising administering to the subject a therapeutically effective amount of a compound of claim 1, or a pharmaceutically acceptable salt thereof.

67. The method of claim 62, wherein the cancer is lymphoma, rhabdoid tumor, multiple myeloma, uterine cancer, gastric cancer, peripheral nervous system cancer, rhabdomyosarcoma, bone cancer, colorectal cancer, mesothelioma, breast cancer, ovarian cancer, lung cancer, fibroblast cancer, central nervous system cancer, urinary tract cancer, upper aerodigestive cancer, leukemia, kidney cancer, skin cancer, esophageal cancer, and pancreatic cancer.

68-71. (canceled)
